(19) United States
(12) Patent Application Publication
(10) Pub. No.: US 2025/0115569 A1
(43) Pub. Date: Apr. 10, 2025

(54) SULFONAMIDE COMPOUNDS FOR THE TREATMENT OF NEUROLOGICAL CONDITIONS

(71) Applicant: LARIO THERAPEUTICS LIMITED, Edinburgh (GB)

(72) Inventors: Henning STEINHAGEN, Schwalbach (DE); Paolo PEVARELLO, Pavia (IT); Maria Pia CATALANI, Verona (IT)

(21) Appl. No.: 18/713,009

(22) PCT Filed: Nov. 25, 2022
(86) PCT No.: PCT/GB2022/053000

§ 371 Date: May 23, 2024

(30) Foreign Application Priority Data
Nov. 26, 2021 (GB) 2117127.7

(51) Int. Cl. C07D 333/34; A61K 31/343; A61K 31/351; A61K 31/353; A61K 31/36; A61K 31/381; A61K 31/397; A61K 31/416; A61K 31/4184; A61K 31/4192; A61K 31/426; A61K 31/428; A61K 31/437; A61K 31/44; A61K 31/4412; A61K 31/4427; A61K 31/4439; A61K 31/4965; A61K 31/4985; A61K 31/50; A61K 31/5025; A61K 31/505; A61K 31/519; A61K 31/5377; C07D 213/71; C07D 213/84; C07D 231/56; C07D 235/14; C07D 237/18; C07D 239/38; C07D 241/18; C07D 249/18; C07D 277/56; C07D 277/82; C07D 307/81; C07D 309/06; C07D 311/58; C07D 317/62; C07D 333/38; C07D 333/60; C07D 405/04; C07D 409/04; C07D 471/04; C07D 487/04; C07D 513/04

(52) U.S. Cl. CPC C07D 333/34; A61K 31/343; A61K 31/351; A61K 31/353; A61K 31/36; A61K 31/381; A61K 31/397; A61K 31/416; A61K 31/4184; A61K 31/4192; A61K 31/426; A61K 31/428; A61K 31/437; A61K 31/44; A61K 31/4412; A61K 31/4427; A61K 31/4439; A61K 31/4965; A61K 31/4985; A61K 31/50; A61K 31/5025; A61K 31/505; A61K 31/519; A61K 31/5377; C07D 213/71; C07D 213/84; C07D 231/56; C07D 235/14; C07D 237/18; C07D 239/38; C07D 241/18; C07D 249/18; C07D 277/56; C07D 277/82; C07D 307/81; C07D 309/06; C07D 311/58; C07D 317/62; C07D 333/38; C07D 333/60; C07D 405/04; C07D 409/04; C07D 471/04; C07D 487/04; C07D 513/04

(57) ABSTRACT

Disclosed are compounds of the formula (I) and pharmaceutically acceptable salts thereof wherein Ring A, Ring B, R¹, R², R³ and L are as defined herein. The compounds are antagonists of the resistant (R-type) voltage-gated calcium ion channel Cav 2.3. Also disclosed are pharmaceutical compositions comprising the compounds; and the compounds for use in the treatment of diseases modulated Cav 2.3, including epilepsy, neurodegenerative conditions such as Parkinson's disease, focal, drug-resistant forms of epilepsy, and other neurological disorders such as developmental and epileptic encephalopathies and Fragile X syndrome.

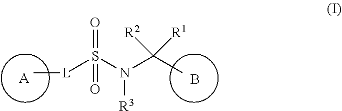

This invention relates to compounds that are antagonists of the resistant (R-type) voltage-gated calcium ion channel Cav 2.3, and the use of the compounds in the treatment and prevention of diseases and conditions associated with Cav2.3, for example neurodegenerative conditions such as Parkinson's disease, focal, drug-resistant forms of epilepsy, and other neurological disorders such as developmental and epileptic encephalopathies and Fragile X syndrome.

### BACKGROUND

Voltage-dependent calcium channels are multi-subunit complexes consisting of alpha-1, alpha-2, beta, and delta subunits in a 1:1:1:1 ratio. Cav2.3 channels belong to the so called pharmaco-resistant or “residual” (R-type) membrane-bound voltage-gated calcium channels and are responsible for calcium ion influx into cells that express them. These channels are structurally only partially characterised. Nevertheless, it is well accepted that most of them are encoded by the CACNA1E gene (Gene ID 777) and are expressed as different Cav2.3 splice variants (variant Cav2.3a to Cav2.3e or f) as the ion conducting subunit (Schneider et al., Pflügers Arch. 2020; 472 (7): 811-816).

Cav2.3 is highly expressed in neuronal and endocrine tissues and has also been detected in heart, kidney, sperm, spleen and retina, and is associated with numerous physiologic and pathophysiologic processes in the central nervous system, vascular system and in endocrine systems (Schneider et al., Pharmaceuticals 2013, 6 (6), 759-776, Schneider et al., Pflügers Arch. 2020; 472 (7): 811-816).

Parkinson disease is the second-most common neurodegenerative disorder that affects 2-3% of the population ≥65 years of age. The primary motor symptoms of Parkinson's are caused by the progressive degeneration of dopaminergic midbrain neurons, particularly those within the substantia nigra (SN) neurones (Giguere et al. 2018, Front. Neurol. 9, 455). This leads to a striatal dopamine deficiency, and intracellular inclusions containing aggregates of α-synuclein are the neuropathological hallmarks of Parkinson disease (Poewe et al, Nat Rev Dis Primers. 2017 Mar. 23; 3:17013). Currently there are no curative therapies available for Parkinson's disease (Bloem et al, Lancet. 2021 Jun. 12; 397 (10291): 2284-2303). Parkinson's disease is a multifactorial disease, and besides genetic risk-factors for Parkinson's disease like PARK-gene mutations, numerous Parkinson's disease-stressors have been identified, including inflammation, viral infections, trauma, gut bacteria, or environmental toxins. Most of these factors lead to mitochondria, proteasomal, and/or lysosomal dysfunction, and elevated metabolic stress, key pathophysiological events in Parkinson's disease. As PARK mutations and also most external factors are global Parkinson's disease-stressors, additional cell-specific features must also contribute to the Parkinson's disease-pathophysiology, and in particular to the differential neuronal vulnerability.

Dopaminergic midbrain neurons display pacemaker activity, which is important for dopamine release and e.g., voluntary movement control. In SN dopaminergic neurons (and other highly vulnerable neurons), this activity generates oscillatory increases in free cytosolic Ca²⁺ levels, which are associated with oscillatory elevated levels of metabolic stress (Guzman et al, Nature. 2010 Dec. 2; 468 (7324): 696-700; Liss & Striessnig, Annu. Rev. Pharmacol Toxicol. 2019 Jan. 6; 59:263-289; Ortner, Front. Synaptic Neurosci. 2021 Feb. 26; 13:636103; Zampese & Surmeier, Cells 2020 Sep. 8; 9 (9): 2045). These increased stresses are thought to render SN neurons more vulnerable to degeneration by Parkinson's disease stressors. Cav2.3 is highly expressed in adult SN dopaminergic neurons and accounts for ˜50% of somatic Ca²⁺ oscillations in SN DA neurons (Benkert et al., 2019, Nat. Commun. 10, 5094).

In patch clamp electrophysiology experiments on brain slices from Cav2.3 knockout mice, the amplitude of the activity-related Ca²⁺ oscillations were significantly reduced by ˜50% in somata of SN neurones compared to wild-type mice. Ca²⁺-dependent action potential after-hyperpolarizations (AHPs), were also significantly reduced in SN dopaminergic neurons of Cav2.3 knockout mice, consistent with the reduction in the Ca²⁺ signals. Similar effects were also observed when Cav2.3 channels were partially blocked using low concentrations of the non-selective peptide antagonist SNX-482, blocking Cav2.3 but also A-type Kv4 potassium channels with protective effects in SN dopaminergic (DA) neurons (Kimm et al, 2014, 34 (28) 9182-9189). Cav 2.3 has also been shown to be implicated the preferential degeneration of these SN DA neurons in an in-vivo model of Parkinson's disease (Benkert et al., 2019, Nat. Commun. 10, 5094).

In a mouse Cav2.3 knockout model in which mice were subjected to low-dose MPTP/probenecid (neurotoxin) knockout of Cav2.3 was shown to provide a significant 100% neuroprotective effect on SN dopaminergic neurons compared to wild-type mice. These data identify Cav2.3 as mediator of SN dopaminergic neuron vulnerability to a degenerative stressor and suggest that Cav2.3 antagonists would be useful in the treatment of Parkinson's disease, for example by providing a neuroprotective treatment of the disease that prevents or inhibits disease progression (Benkert et al., 2019, Nat. Commun. 10, 5094).

In addition to neurodegenerative diseases such as Parkinson's disease Cav2.3 channels are also associated with other diseases and medical disorders, for example Fragile X syndrome (Gray et al., J Neurosci. 2019 Sep. 18; 39 (38): 7453-7464), monogenic developmental and epileptic encephalopathies (DEEs) (Carvill, Epilepsy Curr. May-June 2019; 19 (3): 199-201; Helbig et al., Am J Hum Genet. 2019 Mar. 7; 104 (3): 562; Ortiz Cabrera, Mol Syndromol. 2021 March; 12 (1): 25-32), focal, drug-resistant forms of epilepsy (Weiergraber et al., Epilepsia, 2006, 47:839-50; Weiergraber et al., J. Neurophysiol., 2007, 97:3660-69; Zaman et al., Neuron, 2011, 70:95-108), neurodevelopmental disorders, endocrine disorders such as diabetes (e.g., glucose-induced insulin release, glucose-mediated glucagon suppression, or glucose-mediated somatostatin-release) (Jing et al, 2005, The Journal of clinical investigation 115:146-154, Rorsman et al, 2018, Physiological reviews 98:117-214), the treatment of vasospasm following cerebral aneurism or subarachnoidal haemorrhage (Wang et al., 2010, Journal of Neurotrauma, vol. 27, no. 9, pp. 1723-1732), and pain (for example, chronic pain, inflammatory pain, neuropathic pain (e.g. peripheral neuropathic pain (Shan et al., ACS Chem. Neurosci. 2019, 10, 6, 2939-2955) or central neuropathic pain), or nociceptive pain) (Schneider et al.; Ishiguro et al, Circ. Res. 2005, 96, 419-426, Patel et al., British Journal of Pharmacology 2018, 175, 2173-2184; Wormuth et al., Open Neurol J. 2016; 10:99-126).

WO2018/228692 discloses that Cav2.3 antagonists are beneficial in the neuroprotective treatment of Parkinson's disease and other neurodegenerative diseases.

SNX-482 is a peptide antagonist of Cav2.3 derived from the venom of the tarantula Hysterocratis gigas. SNX-482 has an IC₅₀ of 15-30 nM against Cav2.3, however at higher concentrations SNX-482 also inhibits N-type Ca²⁺ currents Newcomb et al., Biochemistry 1998, 37, 15353-15362); while at similar low nM concentration it inhibits A-Type Kv4 Potassium Currents (Kim et al., J Neurosci. 2014 Jul. 9; 34 (28): 9182-9). The off-target effects of SNX-482 and its general toxicity renders it unsuitable as a neuroprotective treatment for a therapeutic treatment of humans with neurodegenerative conditions such as Parkinson's disease.

Accordingly, there remains a need for Cav2.3 antagonists. Particularly desirable would be Cav2.3 antagonists that are also brain permeable.

### BRIEF SUMMARY OF THE DISCLOSURE

In accordance with the present inventions there is provided a compound of the formula (I), or a pharmaceutically acceptable salt thereof:

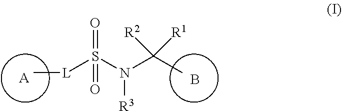

wherein:
R¹ is selected from: C₁₋₆ alkyl, C₃₋₆ cycloalkyl, and C₃₋₆ cycloalkyl-C₁₋₆ alkyl-, wherein R¹ is substituted by at least one fluorine; optionally wherein one or more H in R¹ is substituted by D;
R² is selected from: H, D, C₁₋₆ alkyl and C₁₋₆ haloalkyl; or
   R¹ and R² together with the carbon atom to which they are attached form a C₃₋₆ cycloalkyl substituted with at least one fluorine;
R³ is selected from: C₁₋₆ alkyl and C₁₋₆ haloalkyl; optionally wherein one or more H in R³ is substituted by D;
L is selected from: a bond and C₁₋₃ alkylene;
Ring A is selected from: C₃₋₆ cycloalkyl, 4- to 7-membered heterocyclyl, 5- to 12-membered heteroaryl and C₆₋₁₀ aryl; wherein Ring A is optionally substituted by one or more R⁴;
each R⁴ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
R⁵ and R⁶ are each independently selected from: H, C₁₋₆ alkyl, C₁₋₆ haloalkyl and Q¹,
   wherein said C₁₋₆ alkyl is optionally substituted by one or more R⁸;
each R⁷ and R⁸ is independently selected from: halo, —CN, —OR^{7A}, —S(O)ₓR^{7A}, —NR^{7A}R^{7B}, C(O)R^{7A}, —OC(O)R^{7A}, —C(O)OR^{7A}, —NR^{7A}C(O)R^{7B}, —C(O)NR^{7A}R^{7B} and Q²;
each Q¹ and Q² is independently selected from: C₃₋₆ cycloalkyl, 4- to 7-membered heterocyclyl, phenyl and 5- or 6-membered heteroaryl,
   wherein said C₃₋₆ cycloalkyl, 4- to 7-membered heterocyclyl, phenyl and 5- or 6-membered heteroaryl is optionally substituted by one or more R⁹;
   each R⁹ is independently selected from: halo, ═O, —CN, —NO₂, C₁₋₄ alkyl, C₁₋₄ haloalkyl, —OR^{9A}, —S(O)₂R^{9A}, —NR^{9A}R^{9B}, —C(O)R^{9A}, —OC(O)R^{9A}, —C(O)OR^{9A}, —NR^{9B}C(O)R^{9A}, —C(O)NR^{9A}R^{9B}, —NR^{9B}C(O)OR^{9A}, —OC(O)NR^{9A}R^{9B}, —NR^{9B}SO₂R^{9A} and —SO₂NR^{9A}R^{9B},
   wherein said C₁₋₄ alkyl is optionally substituted by 1 or 2 substituents selected from: halo, —CN, —OR^{9C}, —NR^{9C}R^{9D} and —SO₂R^{9C};
Ring B is phenyl or a 5- or 6-membered heteroaryl, wherein Ring B is optionally substituted by one or more R¹⁰;
   each R¹⁰ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
   each R¹¹ is independently selected from: halo, —CN, —OR^{11A}, —NR^{11A}R^{11B} and —SO₂R^{11A};
R^{7A}, R^{7B}, R^{9A}, R^{9B}, R^{9C}, R^{9D}, R^{10A}, R^{10B}, R^{11A} and R^{11B} are at each occurrence independently selected from: H, C₁₋₄ alkyl and C₁₋₄ haloalkyl;
   and wherein any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
each x is independently 0, 1, or 2;
with the following provisos:
(i) that when the group

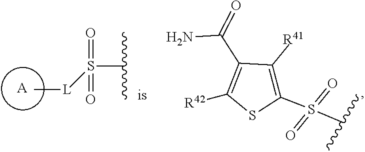

wherein R⁴¹ is H, —CH₃, —CF₃ or cyclopropyl, then R⁴² is not —NHC(O)R⁶; and
(ii) that Compounds A and B are excluded:

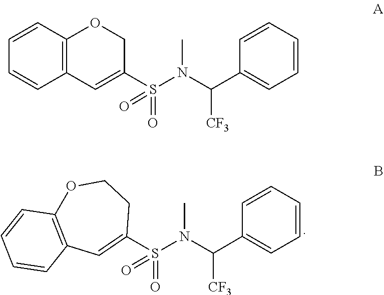 [SMILES: CN(C(c1ccccc1)C(F)(F)F)S(=O)(=O)C1=Cc2ccccc2OC1 ; CN(C(c1ccccc1)C(F)(F)F)S(=O)(=O)C1=Cc2ccccc2OCC1]

Also provided is a pharmaceutical composition comprising a compound of the invention, except that compounds A and B are not excluded, or a pharmaceutically acceptable salt thereof, and a pharmaceutically acceptable excipient.

Also provided is a compound of the invention, except that compounds A and B are not excluded, or a pharmaceutically acceptable salt thereof, for use as a medicament.

Also provided is a compound of the invention, or a pharmaceutically acceptable salt thereof, except that compounds A and B are not excluded, for use in the treatment of a disease or medical disorder mediated by Cav2.3.

Also provided is the use of a compound of the invention, except that compounds A and B are not excluded, or a pharmaceutically acceptable salt thereof, for the manufacture of a medicament for the treatment of a disease or medical disorder mediated by Cav2.3.

Also provided is a method of treating a disease or medical disorder mediated by Cav2.3 in a subject in need thereof, the method comprising administering to the subject an effective amount of a compound of the invention, except that compounds A and B are not excluded, or a pharmaceutically acceptable salt thereof.

In certain embodiments there is provided a compound of the invention, or a pharmaceutically acceptable salt thereof, for use in the prevention or treatment of a disease or medical disorder selected from: a neurodegenerative disease, a neurodevelopmental disorder, epilepsy, an endocrine disorder, cerebral vasospasm, and pain. In certain embodiments there is provided a compound of the invention, or a pharmaceutically acceptable salt thereof for use in the treatment of a neurodegenerative disease, for example Parkinson's disease, Alzheimer's disease, Huntington's disease, dystonia, amyotrophic lateral sclerosis (ALS), and age-related neurodegeneration. Further therapeutic uses of the compounds of the invention are set out in the Detailed Description.

### DETAILED DESCRIPTION

### Definitions

Unless otherwise stated, the following terms used in the specification and claims have the following meanings set out below.

Reference herein to a “compound of the invention” is a reference to any of the compounds disclosed herein including compounds of the formulae (I) to (XXXVIc), a compound selected from compound A. or compound B, or a compound described in any of the Examples, or a pharmaceutically acceptable salt, solvate, or salt of a solvate of any thereof.

The term “antagonist” for example “Cav2.3 antagonist” refers to any molecule that is capable of blocking or decreasing the amount of ions, particularly calcium ions through Cav2.3 channels. An antagonist may prevent of inhibit opening of the channel, or otherwise disrupt the normal operation of the channel. The antagonist may act directly on the channel or indirectly, for example by binding to an allosteric site on the channel.

As used herein, the term “selective antagonist” refers to an antagonist having greater affinity for its target than for one or more related receptors. For example, a “Cav2.3-selective antagonist” has greater affinity for Cav2.3 than for one or more similar calcium-ion channels (e.g., other Cav2, L-type, or N-type family members. The greater affinity of its Cav2.3 target may be, for example, at least: 1.1-fold, 1.2-fold, 1.3-fold, 1.4-fold, 1.5-fold, 2-fold, 3-fold, 4-fold, 5-fold, 10-fold, 20-fold, 50-fold, 100-fold, 1000-fold, 10000-fold, etc. The selectivity of a compound of the invention for Cav2.3 over other ion channels (e.g. one or more other Cav channels selected from Cav1.2, Cav1.2, Cav1.3, Cav1.4, Cav2.1, and Cav 2.2) can be assessed using methods analogous to the Cav2.3 channel calcium-influx assay described herein, using cells which express the channels of interest and comparing the Ic50 values.

The terms “treating”, or “treatment” refer to any beneficial effect in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; modifying the progression of a disease or condition, making the final point of degeneration less debilitating; improving a patient's physical or mental well-being. The treatment or amelioration of symptoms can be based on objective or subjective parameters; including the results of a physical examination, neuropsychiatric examinations, and/or a psychiatric evaluation. The term “treating” and conjugations thereof, includes prevention of an injury, pathology, condition, or disease (i.e., prophylaxis or prevention). For example, the term “treating” and conjugations thereof, include prevention of a pathology, condition, or disease associated with Cav2.3 (e.g., reducing or preventing symptoms or effects of the disease or condition or preventing or inhibiting progression of the disease or condition. For example, a compound of the invention may be for use in preventing, or reducing neurodegeneration in a neurodegenerative disease (e.g. Parkinson's disease), or delaying the onset of symptoms, or delaying the progression of a neurodegenerative disease.

The term “associated” or “associated with”, “involving” or “mediated by” in the context of a Cav2.3 associated with a disease means that the disease is caused by (in whole or in part), or a symptom of the disease is caused by (in whole or in part) by Cav2.3 channels, or channel activity or function. For example, a symptom of a disease or condition associated with Cav2.3 activity may be a symptom that results (entirely or partially) from an increase in the level of activity of Cav2.3 channels and or increased expression of Cav2.3 channels. A disease or medical disorder associated with a Cav2.3 activity or expression, may be treated with a compound of the invention effective for decreasing the level of activity of Cav2.3 channels, for example by blocking or partially blocking the channel, inhibiting the function of the channel, preventing or inhibiting the expression of the channel and/or degrading the channel.

An “effective amount” is an amount sufficient to accomplish a stated purpose. For example an amount sufficient to achieve the effect for which it is administered, treat a disease, reduce enzyme activity, increase enzyme activity, reduce receptor signalling, increase receptor signalling, reduce one or more symptoms of a disease or condition, or to provide a disease modifying effect (i.e. alter the underlying pathophysiology of the disease). An example of an “effective amount” is an amount sufficient to contribute to the treatment, prevention, or reduction of a symptom or symptoms of a disease, or modify the progression of a disease, which could also be referred to as a “therapeutically effective amount.” A “reduction” of a symptom or symptoms means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology, or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The therapeutically effective amount of a compound of the invention can be initially estimated from cell culture assays. Target concentrations will be those concentrations of active compound(s) that are capable of achieving the therapeutic effect described herein, as measured using the methods described herein or known in the art.

Therapeutically effective amounts for use in humans can also be determined from animal models using known methods. For example, a dose for humans can be formulated to achieve a concentration that has been found to be effective in animals. The dosage in humans can be adjusted by monitoring compound effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated, or in response to a biomarker or other correlate or surrogate end-point of the disease. This will provide a therapeutic regimen that is commensurate with the severity of the individual's disease state.

A prophylactic or therapeutic treatment regimen is suitably one that does not cause substantial toxicity and yet is effective to treat the clinical symptoms demonstrated by the particular patient. This determination of a dosage regimen is generally based upon an assessment of the active compound by considering factors such as compound potency, relative bioavailability, patient body weight, presence and severity of adverse side effects, preferred mode of administration and the toxicity profile of the selected agent.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃)CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄ alkoxy, —NR′R″ amino, wherein R′ and R″ are independently H or alkyl. Other substituents for the alkyl group may alternatively be used.

The term “C₁₋₆ haloalkyl”, e.g., “C₁₋₄ haloalkyl”, refers to a hydrocarbon chain substituted with at least one halogen atom independently chosen at each occurrence, for example fluorine, chlorine, bromine, and iodine. The halogen atom may be present at any position on the hydrocarbon chain. For example, C₁₋₆ haloalkyl may refer to chloromethyl, fluoromethyl, trifluoromethyl, chloroethyl e.g., 1-chloromethyl and 2-chloroethyl, trichloroethyl e.g., 1,2,2-trichloroethyl, 2,2,2-trichloroethyl, fluoroethyl e.g., 1-fluoromethyl and 2-fluoroethyl, trifluoroethyl e.g., 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃, —CH₂CHX₂ or —CX(CH₃)CH₃ wherein X is a halo (e.g., F, Cl, Br, or I). A fluoroalkyl group, i.e., a hydrocarbon chain substituted with at least one fluorine atom (e.g., —CF₃, —CHF₂, —CH₂CF₃ or —CH₂CHF₂).

The term “heteroalkyl,” refers to a stable linear or branched chain alkyl, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group. The heteroalkyl is a non-cyclic group. “2 to 8 membered heteroalkyl” refers to a heteroalkyl in which there are a total of 1, 2, 3, 4, 5, 6, 7 or 8 carbon atoms and heteroatoms (e.g., O, N, P, Si, and S) in the heteroalkyl group. Examples include, but are not limited to: —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—N(CH₃)—CH₃, —CH₂—S—CH₂—CH₃, —CH₂—S(O)—CH₃, —CH₂—S(O)₂—CH₃, —CH₂—CH₂—S—CH₃, —CH₂—CH₂—S(O)—CH₃, —CH₂—CH₂—S(O)₂—CH₃, —CH₂—CH═N—OCH₃, Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example, alkenylene may be —CH═CH—, —CH₂CH═CH—, —CH(CH₃)CH═CH— or —CH₂CH═CH—. Alkenyl and alkenylene groups may unsubstituted or substituted by one or more substituents. Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —C≡C—, —CH₂C≡C—, —CH₂C≡CCH₂—, —CH(CH₃)CH≡C— or —CH₂C≡CCH₃. Alkynyl and alkynylene groups may unsubstituted or substituted by one or more substituents. Possible substituents are described herein. For example, substituents may be those described above as substituents for alkyl groups.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon and/or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person will appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

The term “bridged ring systems” includes ring systems in which two rings share more than two atoms, see for example Advanced Organic Chemistry, by Jerry March 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e., the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way.

“—Cₘ₋ₙ alkyl substituted by —NRR” and “Cₘ₋ₙ alkyl substituted by —OR” similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. An aryl may be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. For example, the “aryl” may be a C₆₋₁₂ aryl, suitably phenyl or naphthyl. The aryl system itself may be substituted with other groups. The term “aryl” also covers partially aromatic bi- or polycyclic ring systems wherein at least one ring is an aromatic ring and one or more of the other ring(s) is a non-aromatic, saturated or partially saturated ring.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings, also referred to as a “fused bicyclic heteroaryl”. Bicyclic heteroaryl groups can be vicinally fused, i.e., where the rings are linked to each other through two adjacent carbon and/or nitrogen atoms. Each ring may contain up to about four heteroatoms typically selected from nitrogen, sulfur and oxygen. Typically, the heteroaryl ring will contain up to 4, for example up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl, imidazo[1,2-a]pyridine, imidazo[1,2-a]pyrazine, imidazo[1,2-a]pyrimidine, imidazo[1,2-b]pyridazine, triazolo[1,5-a]pyridine, [1,2,3]triazolo[1,5-a]pyridine. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl.

“Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Partially aromatic heteroaryl bicyclic ring systems can be vicinally fused, i.e., where the rings are linked to each other through two adjacent carbon and/or nitrogen atoms. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 1,3-dihydroisobenzofuran, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “═O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups, which may be the same or different. For example, “one or more optional substituents” may refer to 1 or 2 or 3 substituents (e.g. 1 substituent or 2 substituents).

Where a moiety is substituted, it may be substituted at any point on the moiety where chemically possible and consistent with atomic valency requirements. The moiety may be substituted by one or more substituents, e.g., 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not. For example, it will be recognised that when Ring A is pyridyl the ring nitrogen is not substituted and the ring may be optionally substituted with up to 4 substituents, similarly when Ring A is pyrimidyl, the ring may be optionally substituted up to 3 substituents.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”:

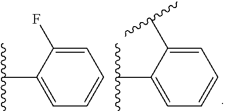

“Meta” substitution is a substitution pattern where two substituents are on carbons one carbon removed from each other, i.e., with a single carbon atom between the substituted carbons. In other words, there is a substituent on the second atom away from the atom with another substituent. For example, the groups below are meta substituted:

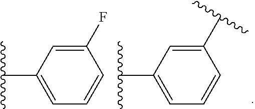

“Para” substitution is a substitution pattern where two substituents are on carbons two carbons removed from each other, i.e., with two carbon atoms between the substituted carbons. In other words, there is a substituent on the third atom away from the atom with another substituent. For example, the groups below are para substituted:

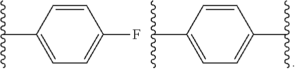

Where Ring A comprises an NH group the NH group may be substituted by R⁴ to give NR⁴. Similarly, where Ring B comprises an NH group, the NH group may be substituted by R¹⁰ to give NR¹⁰.

Reference to a —NRR′ group forming a 4 to 6 membered heterocyclyl refers to R and R′ together with the nitrogen atom to which they are attached forming a 4 to 6 membered heterocyclyl group. For example, a —NR⁵R⁶, —NR^{7A}R^{7B}, —NR^{9A}R^{9B}, —NR^{9C}R^{9D}, —NR^{10A}R^{10B}, and —NR^{11A}R^{11B} group may form:

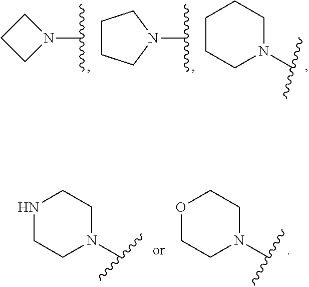

Similarly, an —NRR′ group within a substituent may form a carbonyl-linked 4 to 6 membered heterocyclyl, for example a —C(O)NRR′ group may form:

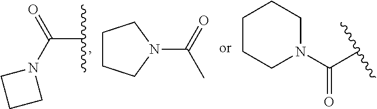

—NRR′ groups within substituents such as —OC(O)NRR′, —SO₂NRR′, or —NRC(O)NRR′, may similarly form a 4 to 6 membered heterocyclyl within such substituents.

A bond terminating in a “ [symbol] ”, i.e. “

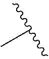

”, represents that the bond is connected to another atom that is not shown in the structure. A bond terminating inside a cyclic structure and not terminating at an atom of the ring structure represents that the bond may be connected to any of the atoms in the ring structure where allowed by valency, unless stated otherwise herein. For example if Ring A is

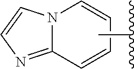

the ring may be attached to the remainder of the compound via either the 5 membered ring or the 6-membered ring, including, but not limited to

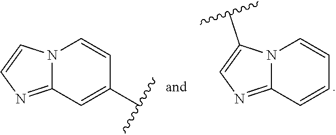 [SMILES: *c1ccn2ccnc2c1 ; *c1cnc2ccccn12]

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and(S) stereoisomers is contemplated. The combination of (R) and(S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer, the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95%, at least 99%, or at least 99.9%.

The compounds of this invention may possess one or more asymmetric centres; such compounds can therefore be produced as individual (R) or(S) stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof

Z/E (e.g., cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g., 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include 2H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. Accordingly, in a compound of the invention one or more hydrogen atom is optionally replaced by deuterium. For example, one or more hydrogen atoms in a C₁₋₄-alkyl group may be replaced by deuterium to form a deuterated C₁₋₄-alkyl group. By way of example, if any of R¹, R³, R⁴ or R¹⁰ is methyl the invention also encompasses —CD₃, —CHD₂ and —CH₂D. Similarly R² may be D.

Certain compounds of the invention may exist in solvated as well as unsolvated forms such as, for example, hydrated forms. It is to be understood that the invention encompasses all such solvated forms.

It is also to be understood that certain compounds of the invention may exhibit polymorphism, and that the invention encompasses all such forms.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

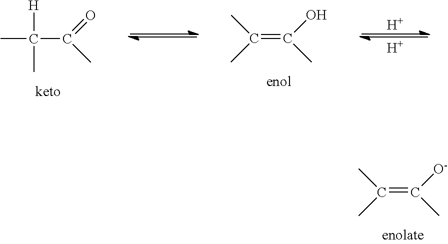

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e., a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo-cleavable ester derivatives that may be formed at a carboxy group or a hydroxy group in a compound of the invention and in vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula (I) that possesses a carboxy group is, for example, an in vivo-cleavable ester thereof. An in vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically-acceptable pro-drug of a compound of the invention that possesses a hydroxy group is, for example, an in vivo-cleavable ester or ether thereof. An in vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl) piperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the invention that possesses a carboxy group is, for example, an in vivo-cleavable amide thereof, for example an amide formed with an amine such as ammonia, a C₁₋₄ alkylamine such as methylamine, a (C₁₋₄ alkyl)₂amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in vivo-cleavable amide or carbamate derivative thereof. Suitable pharmaceutically-acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl) piperazin-1-ylmethyl. Suitable pharmaceutically-acceptable carbamates from an amino group include, for example acyloxyalkoxycarbonyl and benzyloxycarbonyl groups.

Throughout the description and claims of this specification, the words “comprise” and “contain” and variations of them mean “including but not limited to”, and they are not intended to (and do not) exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Compounds

The following paragraphs are applicable to the compounds of the invention, including compounds of the formulae (I) to XXXVIc.

In certain embodiments the compound of the formula (I) is a compound of the formula (Ia), or a pharmaceutically acceptable salt thereof:

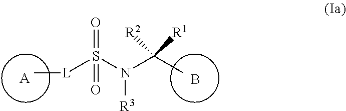

In certain embodiments the compound of the formula (I) is a compound of the formula (II), or a pharmaceutically acceptable salt thereof:

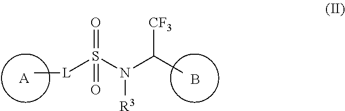

wherein
R³, L, Ring A, and Ring B are as defined for formula (I).

In certain embodiments the compound of the formula (II) is a compound of the formula (IIa), or a pharmaceutically acceptable salt thereof:

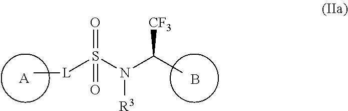

In certain embodiments the compound of the formula (I) is a compound of the formula (III), or a pharmaceutically acceptable salt thereof:

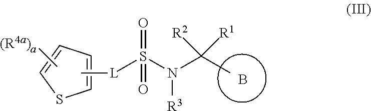

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, C₁₋₆ heteroalkyl, C₂₋₆ alkenyl and C₂₋₆ alkynyl is optionally substituted by one or more R⁷;
a is an integer from 0 to 3; and
R¹, R², R³, R⁵, R⁶, R⁷, Q¹, L, x, and Ring B are as defined for formula (I).

In certain embodiments the compound of the formula (III) is a compound of the formula (IIIa), or a pharmaceutically acceptable salt thereof:

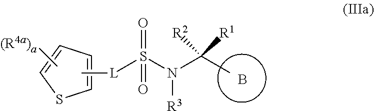

In certain embodiments the compound of the formula (I) is a compound of the formula (IV), or a pharmaceutically acceptable salt thereof:

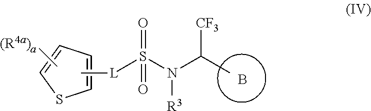

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3; and
R³, R⁵, R⁶, R⁷, Q¹, L, x, and Ring B are as defined for formula (I).

In certain embodiments the compound of the formula (IV) is a compound of the formula (IVa), or a pharmaceutically acceptable salt thereof:

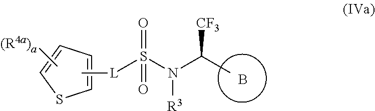

In certain embodiments the compound of the formula (I) is a compound of the formula (V), or a pharmaceutically acceptable salt thereof:

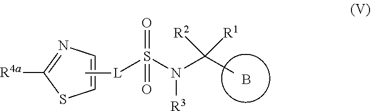

wherein:
each R^{4a} is independently selected from: H, halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
R¹, R², R³, R⁵, R⁶, R⁷, Q¹, L, x, and Ring B are as defined for formula (I).

In certain embodiments the compound of the formula (V) is a compound of the formula (Va), or a pharmaceutically acceptable salt thereof:

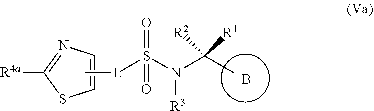

In certain embodiments the compound of the formula (I) is a compound of the formula (VI), or a pharmaceutically acceptable salt thereof:

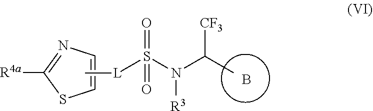

wherein:
each R^{4a} is independently selected from: H, halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
R³, R⁵, R⁶, R⁷, Q¹, L, x, and Ring B are as defined for formula (I).

In certain embodiments the compound of the formula (VI) is a compound of the formula (VIa), or a pharmaceutically acceptable salt thereof:

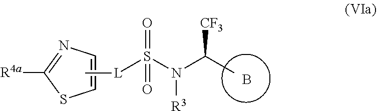

In certain embodiments the compound of the formula (I) is a compound of the formula (VII), or a pharmaceutically acceptable salt thereof:

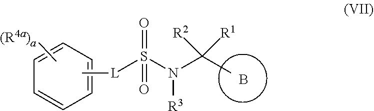

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 5; and
R¹, R², R³, R⁵, R⁶, R⁷, Q¹, L, x, and Ring B are as defined for formula (I).

In certain embodiments the compound of the formula (VII) is a compound of the formula (VIIa), or a pharmaceutically acceptable salt thereof:

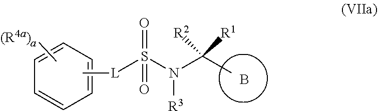

In certain embodiments the compound of the formula (I) is a compound of the formula (VIII), or a pharmaceutically acceptable salt thereof:

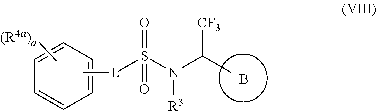

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 5; and
R³, R⁵, R⁶, R⁷, Q¹, L, x, and Ring B are as defined for formula (I).

In certain embodiments the compound of the formula (VIII) is a compound of the formula (VIIIa), or a pharmaceutically acceptable salt thereof:

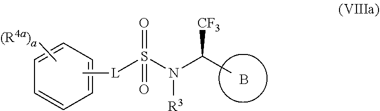

In certain embodiments the compound of the formula (I) is a compound of the formula (IX), or a pharmaceutically acceptable salt thereof:

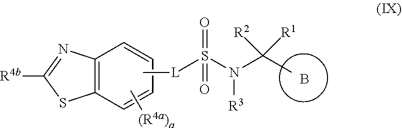

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3;
each R^{4b} is independently selected from: H, halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
R¹, R², R³, R⁵, R⁶, R⁷, Q¹, L, x, and Ring B are as defined for formula (I).

In certain embodiments the compound of the formula (IX) is a compound of the formula (IXa), or a pharmaceutically acceptable salt thereof:

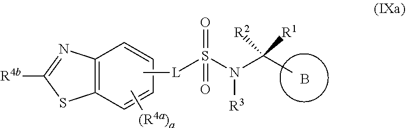

In certain embodiments the compound of the formula (I) is a compound of the formula (X), or a pharmaceutically acceptable salt thereof:

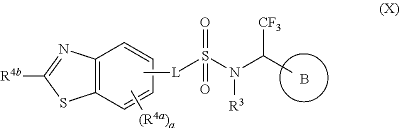

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3;
each R^{4b} is independently selected from: H, halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
R³, R⁵, R⁶, R⁷, Q¹, L, x, and Ring B are as defined for formula (I).

In certain embodiments the compound of the formula (X) is a compound of the formula (Xa), or a pharmaceutically acceptable salt thereof:

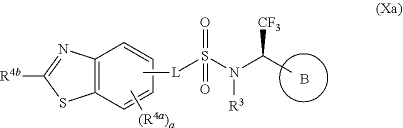

In certain embodiments the compound of the formula (I) is a compound of the formula (XI), or a pharmaceutically acceptable salt thereof:

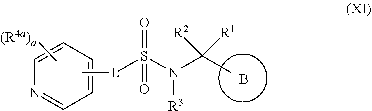

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 4; and
R¹, R², R³, R⁵, R⁶, R⁷, Q¹, L, x, and Ring B are as defined for formula (I).

In certain embodiments the compound of the formula (XI) is a compound of the formula (XIa), or a pharmaceutically acceptable salt thereof:

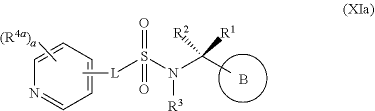

In certain embodiments the compound of the formula (I) is a compound of the formula (XII), or a pharmaceutically acceptable salt thereof:

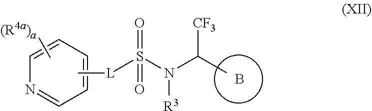

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 4; and
R³, R⁵, R⁶, R⁷, Q¹, L, x, and Ring B are as defined for formula (I).

In certain embodiments the compound of the formula (XII) is a compound of the formula (XIIa), or a pharmaceutically acceptable salt thereof:

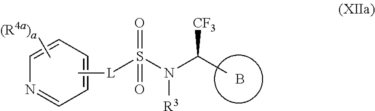

In certain embodiments the compound of the formula (I) is a compound of the formula (XIII), or a pharmaceutically acceptable salt thereof:

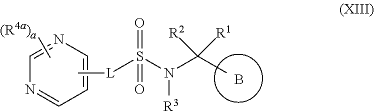

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3; and
R¹, R², R³, R⁵, R⁶, R⁷, Q¹, L, x, and Ring B are as defined for formula (I).

In certain embodiments the compound of the formula (XIII) is a compound of the formula (XIIIa), or a pharmaceutically acceptable salt thereof:

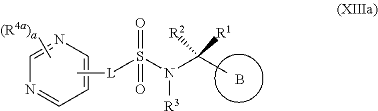

In certain embodiments the compound of the formula (I) is a compound of the formula (XIV), or a pharmaceutically acceptable salt thereof:

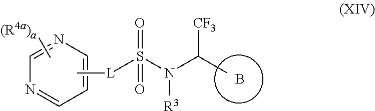

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3; and
R³, R⁵, R⁶, R⁷, Q¹, L, x, and Ring B are as defined for formula (I).

In certain embodiments the compound of the formula (XIV) is a compound of the formula (XIVa), or a pharmaceutically acceptable salt thereof:

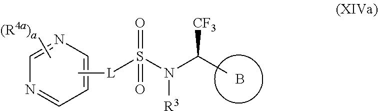

In certain embodiments the compound of the formula (I) is a compound of the formula (XV), or a pharmaceutically acceptable salt thereof:

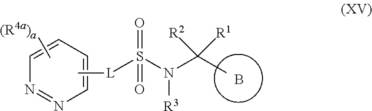

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3; and
R¹, R², R³, R⁵, R⁶, R⁷, Q¹, L, x, and Ring B are as defined for formula (I).

In certain embodiments the compound of the formula (XV) is a compound of the formula (XVa), or a pharmaceutically acceptable salt thereof:

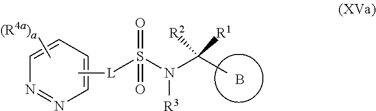

In certain embodiments the compound of the formula (I) is a compound of the formula (XVI), or a pharmaceutically acceptable salt thereof:

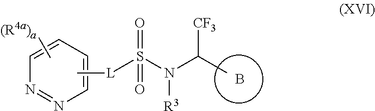

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3; and
R³, R⁵, R⁶, R⁷, Q¹, L, x, and Ring B are as defined for formula (I).

In certain embodiments the compound of the formula (XVI) is a compound of the formula (XIVa), or a pharmaceutically acceptable salt thereof:

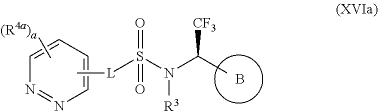

In certain embodiments the compound of the formula (I) is a compound of the formula (XVII), or a pharmaceutically acceptable salt thereof:

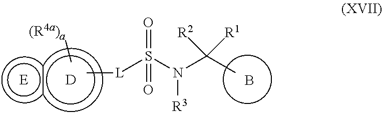

wherein:
Ring D is phenyl or a 6-membered heteroaryl containing 1, 2 or 3 ring nitrogen atoms and Ring E is a 5-membered heteroaryl containing 1 or more (e.g. 1 to 4) ring nitrogen atoms and optionally one or two ring heteroatoms selected from O and S, and wherein Rings D and E together form a 9-membered fused bicyclic heteroaryl ring;
L is attached to an atom in Ring D;
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 4; and
R¹, R², R³, R⁵, R⁶, R⁷, Q¹, L, x, and Ring B are as defined for formula (I).

In certain embodiments the compound of the formula (XVII) is a compound of the formula (XVIIa), or a pharmaceutically acceptable salt thereof:

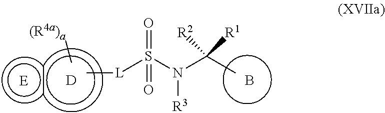

In certain embodiments the compound of the formula (XVII) is a compound of the formula (XVIII), or a pharmaceutically acceptable salt thereof:

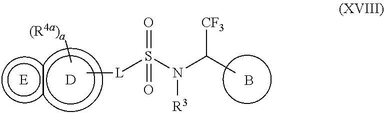

In certain embodiments the compound of the formula (XVIII) is a compound of the formula (XVIIIa), or a pharmaceutically acceptable salt thereof:

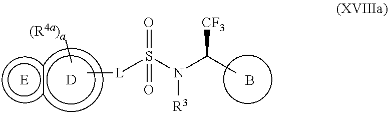

In certain embodiments the compound of the formula (I) is a compound of the formula (XIX), or a pharmaceutically acceptable salt thereof:

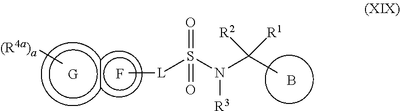

wherein:
Ring F is a 5-membered heteroaryl containing 1 or more (e.g. 1 to 3) ring nitrogen atoms and optionally one or two ring heteroatoms selected from O and S, Ring G is phenyl or a 6-membered heteroaryl containing 1, 2 or 3 ring nitrogen atoms, and wherein Rings F and G together form a 9-membered fused bicyclic heteroaryl ring;
L is attached to an atom in Ring F;
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 4; and
R¹, R², R³, R⁵, R⁶, R⁷, Q¹, L, x, and Ring B are as defined for formula (I).

In certain embodiments the compound of the formula (XIX) is a compound of the formula (XIXa), or a pharmaceutically acceptable salt thereof:

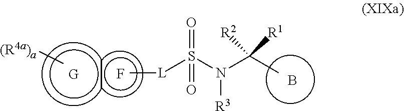

In certain embodiments the compound of the formula (XIX) is a compound of the formula (XX), or a pharmaceutically acceptable salt thereof:

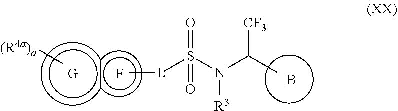

In certain embodiments the compound of the formula (XX) is a compound of the formula (XXa), or a pharmaceutically acceptable salt thereof:

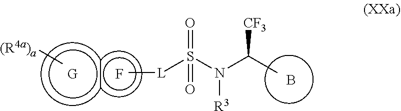

In certain embodiments the compound of the formula (I) is a compound of the formula (XXI), or a pharmaceutically acceptable salt thereof:

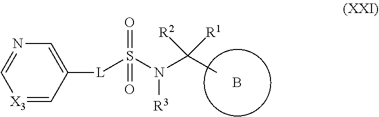

wherein:
X₃ is N or CR^{4a};
R^{4a} is selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
R¹, R², R³, R⁵, R⁶, R⁷, Q¹, L, x, and Ring B are as defined for formula (I).

In certain embodiments the compound of the formula (XXI) is a compound of the formula (XXIa), or a pharmaceutically acceptable salt thereof:

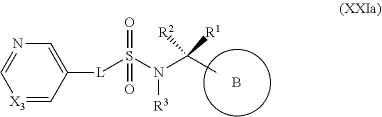

In certain embodiments the compound of the formula (I) is a compound of the formula (XXII), or a pharmaceutically acceptable salt thereof:

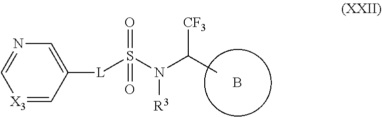

wherein:
X₃ is N or CR^{4a};
R^{4a} is selected from: H, halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
R³, R⁵, R⁶, R⁷, Q¹, L, x, and Ring B are as defined for formula (I).

In certain embodiments the compound of the formula (XXII) is a compound of the formula (XXIIa), or a pharmaceutically acceptable salt thereof:

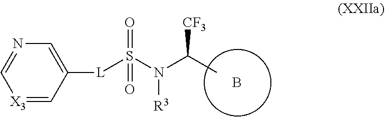

In certain embodiments the compound of the formula (I) is a compound of the formula (XXIII), or a pharmaceutically acceptable salt thereof:

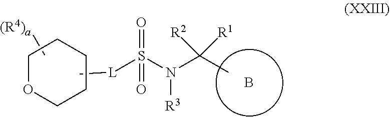

wherein:
a is an integer from 0 to 4; and R¹, R², R³, R⁴, L and Ring B are as defined for formula (I).

In certain embodiments the compound of the formula (XIII) is a compound of the formula (XXIIIa), or a pharmaceutically acceptable salt thereof:

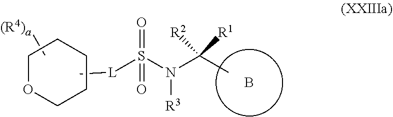

In certain embodiments the compound of the formula (I) is a compound of the formula (XXIV), or a pharmaceutically acceptable salt thereof:

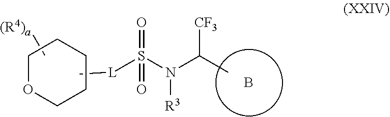

wherein:
a is an integer from 0 to 4; and R³, R⁴, L and Ring B are as defined for formula (I).

In certain embodiments the compound of the formula (XXIV) is a compound of the formula (XXIVa), or a pharmaceutically acceptable salt thereof:

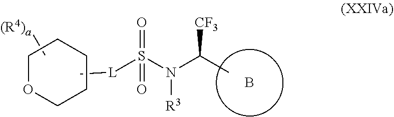

In certain embodiments the compound of the formula (I) is a compound of the formula (XXV), or a pharmaceutically acceptable salt thereof:

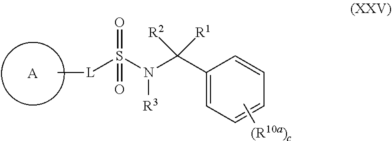

wherein
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B}, —
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R^{10A}, R^{10B}, R¹¹, L, x, and Ring A are as defined for formula (I).

In certain embodiments the compound of the formula (XXV) is a compound of the formula (XXVa), or a pharmaceutically acceptable salt thereof:

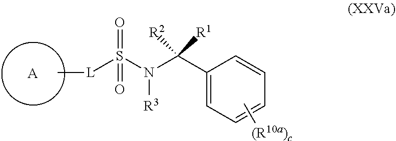

In certain embodiments the compound of the formula (I) is a compound of the formula (XXVI), or a pharmaceutically acceptable salt thereof:

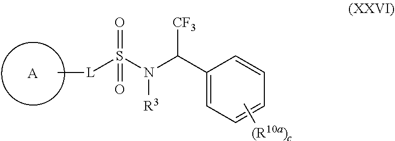

wherein
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R³, R^{10A}, R^{10B}, R¹¹, L, x, and Ring A are as defined for formula (I).

In certain embodiments the compound of the formula (XXVI) is a compound of the formula (XXVIa), or a pharmaceutically acceptable salt thereof:

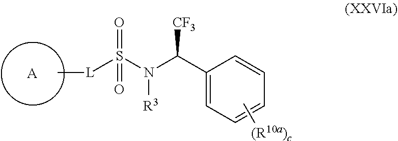

In certain embodiments the compound of the formula (I) is a compound of the formula (XXVII), or a pharmaceutically acceptable salt thereof:

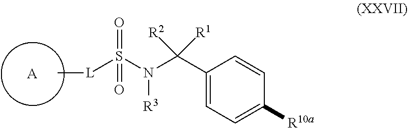

wherein
R^{10a} is selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
R¹, R², R³, R^{10A}, R^{10B}, R¹¹, L, x, and Ring A are as defined for formula (I).

In certain embodiments the compound of the formula (XXVII) is a compound of the formula (XXVIIa), or a pharmaceutically acceptable salt thereof:

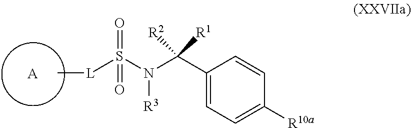

In certain embodiments the compound of the formula (I) is a compound of the formula (XXVIII), or a pharmaceutically acceptable salt thereof:

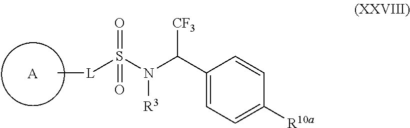

wherein:
R^{10a} is selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹; and

R³, R^{10A}, R^{10B}, R¹¹, L, x, and Ring A are as defined for formula (I).

In certain embodiments the compound of the formula (XXVIII) is a compound of the formula (XXVIIIa), or a pharmaceutically acceptable salt thereof:

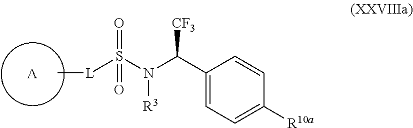

In certain embodiments the compound of the formula (I) is a compound of the formula (XXIX), or a pharmaceutically acceptable salt thereof:

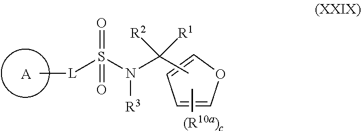

wherein:
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 3; and
R¹, R², R³, R^{10A}, R^{10B}, R¹¹, L, x, and Ring A are as defined for formula (I).

In certain embodiments the compound of the formula (I) is a compound of the formula (XXX), or a pharmaceutically acceptable salt thereof:

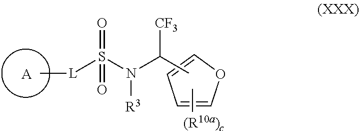

wherein
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 3; and
R³, R^{10A}, R^{10B}, R¹¹, L, x, and Ring A are as defined for formula (I).

In certain embodiments the compound of the formula (XXX) is a compound of the formula (XXXa), or a pharmaceutically acceptable salt thereof:

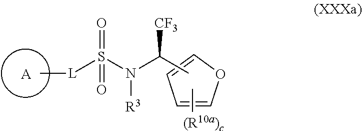

In certain embodiments the compound of the formula (I) is a compound of the formula (XXXI), or a pharmaceutically acceptable salt thereof:

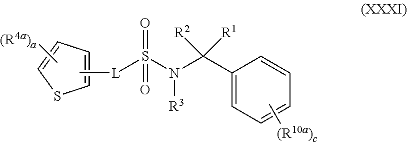

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3;
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R⁵, R⁶, R⁷, R^{10A}, R^{10B}, R¹¹, Q¹, L, and x are as defined for formula (I).

In certain embodiments the compound of the formula (XXXI) is a compound of the formula (XXXIa), or a pharmaceutically acceptable salt thereof:

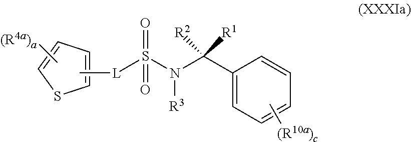

In certain embodiments the compound of the formula (XXXI) is a compound of the formula (XXXIb), or a pharmaceutically acceptable salt thereof:

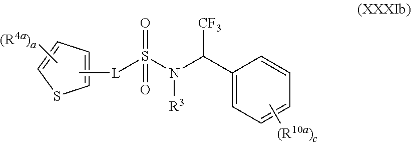

In certain embodiments the compound of the formula (XXXI) is a compound of the formula (XXXIc), or a pharmaceutically acceptable salt thereof:

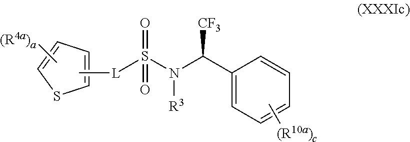

In certain embodiments the compound of the formula (I) is a compound of the formula (XXXII), or a pharmaceutically acceptable salt thereof:

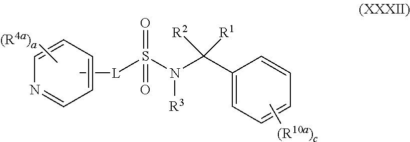

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 4;
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R⁵, R⁶, R⁷, R^{10A}, R^{10B}, R¹¹, Q¹, L, and x are as defined for formula (I).

In certain embodiments the compound of the formula (XXXII) is a compound of the formula (XXXIIa), or a pharmaceutically acceptable salt thereof:

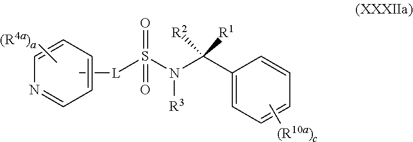

In certain embodiments the compound of the formula (XXXII) is a compound of the formula (XXXIIb), or a pharmaceutically acceptable salt thereof:

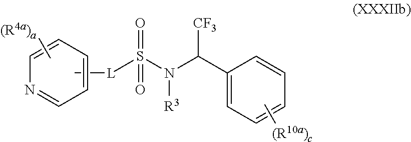

In certain embodiments the compound of the formula (XXXII) is a compound of the formula (XXXIIc), or a pharmaceutically acceptable salt thereof:

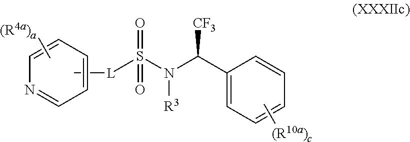

In certain embodiments the compound of the formula (I) is a compound of the formula (XXXIII), or a pharmaceutically acceptable salt thereof:

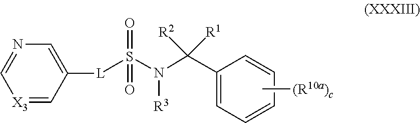

wherein:
X₃ is N or CR^{4a};
R^{4a} is selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R⁵, R⁶, R⁷, R^{10A}, R^{10B}, R¹¹, Q¹, L, and x are as defined for formula (I).

In certain embodiments the compound of the formula (XXXIII) is a compound of the formula (XXXIIIa), or a pharmaceutically acceptable salt thereof:

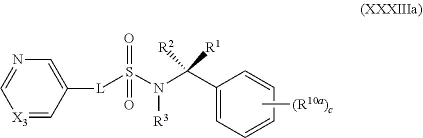

In certain embodiments the compound of the formula (XXXIII) is a compound of the formula (XXXIIIb), or a pharmaceutically acceptable salt thereof:

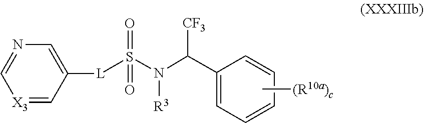

In certain embodiments the compound of the formula (XXXIII) is a compound of the formula (XXXIIIc), or a pharmaceutically acceptable salt thereof:

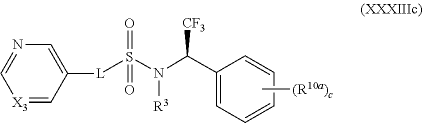

In certain embodiments the compound of the formula (I) is a compound of the formula (XXXIV), or a pharmaceutically acceptable salt thereof:

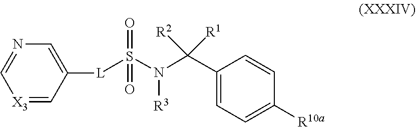

wherein:
X₃ is N or CR^{4a};
R^{4a} is selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶
   wherein said C₁₋₆ alkyl, 2 to 8 membered heteroalkyl, C₂₋₆ alkenyl and C₂₋₆ alkynyl is optionally substituted by one or more R⁷;
R^{10a} is selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
R¹, R², R³, R⁵, R⁶, R⁷, R^{10A}, R^{10B}, R¹¹, Q¹, L, and x are as defined for formula (I).

In certain embodiments the compound of the formula (XXXIV) is a compound of the formula (XXXIVa), or a pharmaceutically acceptable salt thereof:

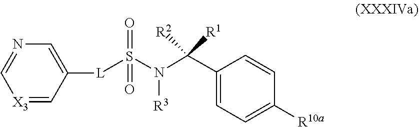

In certain embodiments the compound of the formula (XXXIV) is a compound of the formula (XXXIVb), or a pharmaceutically acceptable salt thereof:

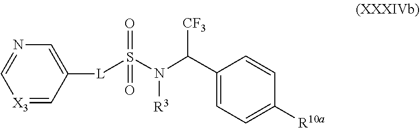

In certain embodiments the compound of the formula (XXXIV) is a compound of the formula (XXXIVc), or a pharmaceutically acceptable salt thereof:

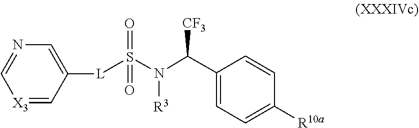

In certain embodiments the compound of the formula (I) is a compound of the formula (XXXV), or a pharmaceutically acceptable salt thereof:

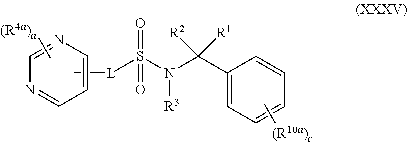

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3;
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R⁵, R⁶, R⁷, R^{10A}, R^{10B}, R¹¹, Q¹, L, and x are as defined for formula (I).

In certain embodiments the compound of the formula (XXXV) is a compound of the formula (XXXVa), or a pharmaceutically acceptable salt thereof:

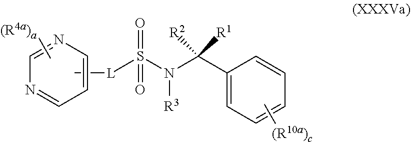

In certain embodiments the compound of the formula (XXXV) is a compound of the formula (XXXVb), or a pharmaceutically acceptable salt thereof:

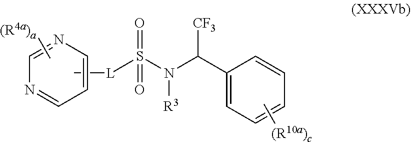

In certain embodiments the compound of the formula (XXXV) is a compound of the formula (XXXVc), or a pharmaceutically acceptable salt thereof:

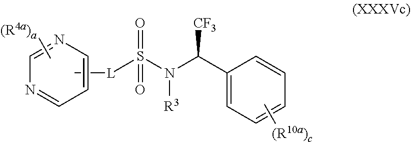

In certain embodiments the compound of the formula (I) is a compound of the formula (XXXVI), or a pharmaceutically acceptable salt thereof:

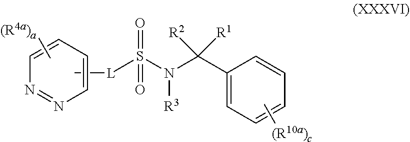

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3;
each R^{10a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
c is an integer from 0 to 5; and
R¹, R², R³, R⁵, R⁶, R⁷, R^{10A}, R^{10B}, R¹¹, Q¹, L, and x are as defined for formula (I).

In certain embodiments the compound of the formula (XXXVI) is a compound of the formula (XXXVIa), or a pharmaceutically acceptable salt thereof:

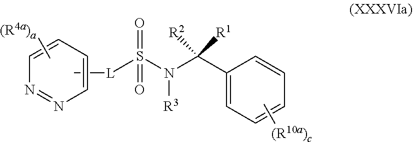

In certain embodiments the compound of the formula (XXXVI) is a compound of the formula (XXXVIb), or a pharmaceutically acceptable salt thereof:

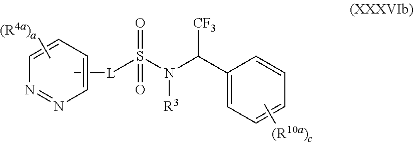

In certain embodiments the compound of the formula (XXXVI) is a compound of the formula (XXXVIc), or a pharmaceutically acceptable salt thereof:

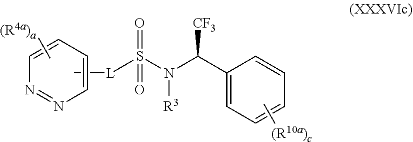

In certain embodiments compounds of the invention include, for example, compounds of formulae (I) to (XXXVIc), or a pharmaceutically acceptable salt thereof, wherein, unless otherwise stated, each of Ring A, Ring B, R¹, R², R³, R⁴, R^{4a}, R^{4b}, R⁵, R⁶, R⁷, R⁸, R⁹, R¹⁰, R¹¹, R^{7A}, R^{7B}, R^{9A}, R^{9B}, R^{9C}, R^{9D}, R^{10A}, R^{10B}, R^{11A}, R^{11B}, Q¹, Q², L, a, c, and x has any of the meanings defined hereinbefore or in any of the following statements in the numbered paragraphs 1 to 223 hereinafter. These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.
1. R¹ is selected from: C₁₋₆ alkyl, C₃₋₆ cycloalkyl, and C₃₋₆ cycloalkyl-C₁₋₆ alkyl-, wherein R¹ is substituted by at least one fluorine.
2. R¹ is selected from: C₁₋₃ alkyl, C₃₋₆ cycloalkyl, and C₃₋₆ cycloalkyl-C₁₋₃ alkyl-, wherein R¹ is substituted by at least one fluorine.
3. R¹ is selected from: C₁₋₆ alkyl, and C₃₋₆ cycloalkyl, wherein R¹ is substituted by at least one fluorine.
4. R¹ is selected from: C₁₋₃ alkyl, and C₃₋₆ cycloalkyl, wherein R¹ is substituted by at least one fluorine.
5. R¹ is C₁₋₆ alkyl, wherein R¹ is substituted by at least one fluorine.
6. R¹ is C₁₋₃ alkyl, wherein R¹ is substituted by at least one fluorine.
7. R¹ is selected from ethyl and methyl, wherein the ethyl or methyl is substituted by at least one fluorine.
8. R¹ is ethyl wherein the ethyl is substituted by at least one fluorine.
9. R¹ is selected from —CH₂F, —CHF₂, and —CF₃.
10. R¹ is —CH₂F.
11. R¹ is —CHF₂.
12. R¹ is —CF₃.
13. R² is selected from: H, C₁₋₃ alkyl and C₁₋₃ haloalkyl.
14. R² is selected from: H, and C₁₋₃ alkyl.
15. R² is C₁₋₃ alkyl.
16. R² is methyl.
17. R² is H or D. It may be that R² is D. It may be that R² is H.
18. R¹ and R² together with the carbon atom to which they are attached form a C₃ or C₄ cycloalkyl substituted with at least one fluorine.
19. R¹ and R² together with the carbon atom to which they are attached form a cyclobutyl group substituted with at least one fluorine. Thus it may be that R¹ and R² together with the carbon atom to which they are attached form a cyclobutyl group substituted one fluorine.
20. R¹ and R² together with the carbon atom to which they are attached form a cyclopropyl group substituted with at least one fluorine. Thus it may be that R¹ and R² together with the carbon atom to which they are attached form a cyclopropyl group substituted with one fluorine.

21. R¹ is as defined in any of 1 to 12 and R² is methyl.
22. R¹ is as defined in any of 1 to 12 and R² is H.
23. R³ is selected from: C₁₋₃ alkyl and C₁₋₃ haloalkyl.
24. R³ is selected from: C₁₋₂ alkyl and C₁₋₂ haloalkyl.
25. R³ is methyl optionally substituted with 1 to 3 halo groups.
26. R³ is ethyl optionally substituted with 1 to 5 halo groups.
27. R³ is as defined in any of 23 to 26, wherein said halo is fluoro.
28. R³ is C₁₋₃ alkyl.
29. R³ is selected from: methyl, ethyl, and 2-fluoroethyl.
30. R³ is methyl.
31. R³ is ethyl.
32. R³ is 2-fluoroethyl.
33. R³ is as defined in any of 23 to 32 wherein one or more hydrogen atoms in R³ is deuterium. Thus it may be that R³ is selected from: methyl, —CD₃, ethyl, and 2-fluoroethyl.
34. R³ is —CD₃.
35. L is selected from: a bond and C₁₋₂ alkyl.
36. L is selected from: a bond, —CH₂—, and —CH₂CH₂—.
37. L is selected from: a bond and —CH₂—.
38. L is —CH₂—.
39. L is a bond.
40. Ring A is selected from: 4- to 7-membered heterocyclyl, 5- to 12-membered heteroaryl and C₆₋₁₀ aryl.
41. Ring A is selected from: 4- to 7-membered heterocyclyl and 5- to 12-membered heteroaryl.
42. Ring A is selected from: 5- or 6-membered heterocyclyl, 5- to 10-membered heteroaryl and C₆₋₈ aryl.
43. Ring A is selected from: 5- or 6-membered heterocyclyl, and 5- to 10-membered heteroaryl.
44. Ring A is selected from: 5- or 6-membered heterocyclyl, 5- to 9-membered heteroaryl and phenyl.
45. Ring A is selected from: 5- or 6-membered heterocyclyl and 5- to 9-membered heteroaryl.
46. Ring A is selected from: 5- to 10-membered heteroaryl and phenyl.
47. Ring A is a 5- to 10-membered heteroaryl.
48. Ring A is a 6- to 10-membered heteroaryl.
49. Ring A is selected from: 5-membered heteroaryl, 6-membered heteroaryl, 9-membered heteroaryl, and phenyl.
50. Ring A is a monocyclic 5- or 6-membered heteroaryl or a 8- to 10-membered fused bicyclic heteroaryl, wherein Ring A has at least 1 (for example 1 to 4) ring nitrogen atom. Thus it may be that Ring A is a monocyclic 6-membered heteroaryl or a 9- to 10-membered fused bicyclic heteroaryl, wherein Ring A has at least 1 (for example 1 to 4) ring nitrogen atom.
51. Ring A is a monocyclic 5- or 6-membered heteroaryl or a 9- to 10-membered fused bicyclic heteroaryl, wherein Ring A has 1 to 4 ring nitrogen atoms. Thus it may be that Ring A is a monocyclic 6-membered heteroaryl or a 9-membered fused bicyclic heteroaryl, wherein Ring A has 1 to 4 ring nitrogen atoms.
52. Ring A is 5-membered heteroaryl.
53. Ring A is 5-membered heteroaryl, wherein Ring A said heteroaryl has 1 ring nitrogen atom and optionally one or more ring heteroatoms (for example 1, 2 or 3) selected from O, S and N.
54. Ring A is 5-membered heteroaryl, wherein said heteroaryl has 1, 2, 3 or 4 ring nitrogen atoms.
55. Ring A is 6-membered heteroaryl.
56. Ring A is a monocyclic 6-membered heteroaryl, wherein said heteroaryl has 1, 2 or 3 (for example 1 or 2) ring nitrogen atoms.
57. Ring A is 9-membered heteroaryl.
58. Ring A is 9-membered fused bicyclic heteroaryl, wherein said heteroaryl has 1, 2, 3 or 4 ring nitrogen atoms.
59. Ring A is 9-membered bicyclic heteroaryl, wherein said heteroaryl has 1, 2, 3 or 4 (for example 1, 2 or 3) ring nitrogen atoms and is a 6-membered ring fused to a 5-membered ring, wherein the 9-membered bicyclic heteroaryl is attached to the group L by a ring atom in the 6-membered ring. It may be that the 5- and 6-membered rings forming the 9-membered bicyclic heteroaryl are both heteroaryl rings.
60. Ring A is 9-membered bicyclic heteroaryl, wherein said heteroaryl has 1, 2, 3 or 4 (for example 1, 2 or 3) ring nitrogen atoms and is a 6-membered ring fused to a 5-membered ring, wherein the 9-membered bicyclic heteroaryl is attached to the group L by a ring atom in the 5-membered ring. It may be that the 5- and 6-membered rings forming the 9-membered bicyclic heteroaryl are both heteroaryl rings.
61. Ring A is selected from: a 6-membered heteroaryl and a 9-membered bicyclic heteroaryl, wherein said 6-membered heteroaryl has 1, 2 or 3 (for example 1 or 2) ring nitrogen atoms, and said 9-membered bicyclic heteroaryl has 1, 2, 3 or 4 (for example 1, 2 or 3) ring nitrogen atoms. It may be that the 5- and 6-membered rings forming the 9-membered bicyclic heteroaryl are both heteroaryl rings.
62. Ring A is phenyl.
63. Ring A is a 4- to 6-membered heterocyclyl.
64. Ring A is a 5- or 6-membered heterocyclyl containing at least one ring oxygen atom and optionally 1 further ring heteroatom selected from S and N.
65. Ring A is selected from cyclopropyl, cyclobutyl, cyclopentyl, cyclohexyl, pyrrolidinyl, tetrahydrofuranyl, piperidinyl, and tetrahydropyranyl.
66. Ring A is selected from pyrrolidinyl, tetrahydrofuranyl, piperidinyl, and tetrahydropyranyl.
67. Ring A is tetrahydropyranyl.
68. Ring A is selected from furanyl, thienyl, imidazolyl, pyrazolyl, oxazolyl, isoxazolyl, thiazolyl, isothiazolyl, thiadiazolyl.
69. Ring A is selected from furanyl, thienyl, imidazolyl, pyrazolyi, oxazolyi, isoxazolyl, thiazolyl, isothiazolyl, thiadiazolyi, phenyl, pyridyl, pyrimidinyl, pyrazinyl, or a compound of the structure:

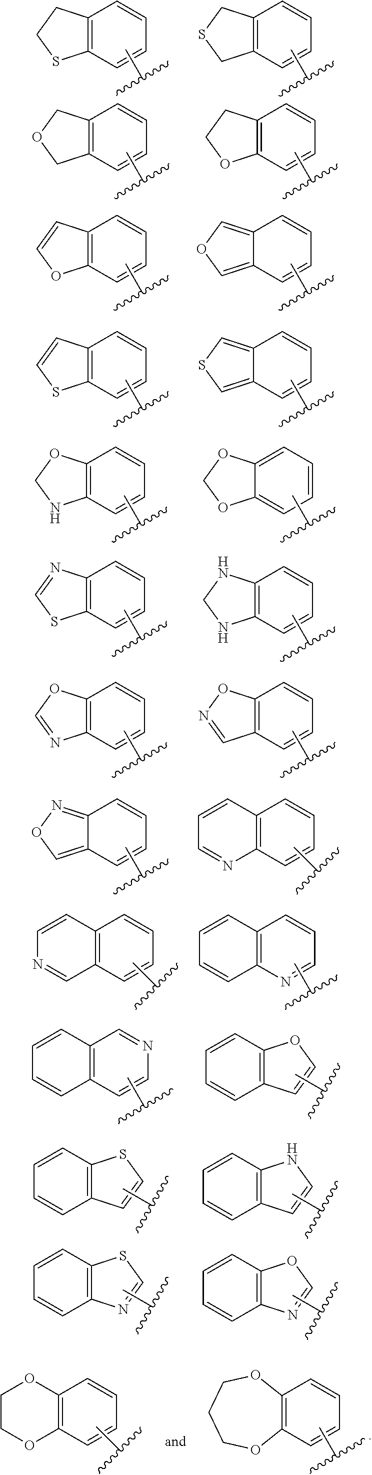

wherein Ring A is optionally substituted with one or more R⁴.
70. Ring A is selected from: thienyl, thiazolyl, pyridyl, pyrimidyl, pyrazinyl, pyridazinyl, tetrahydropyranyl,

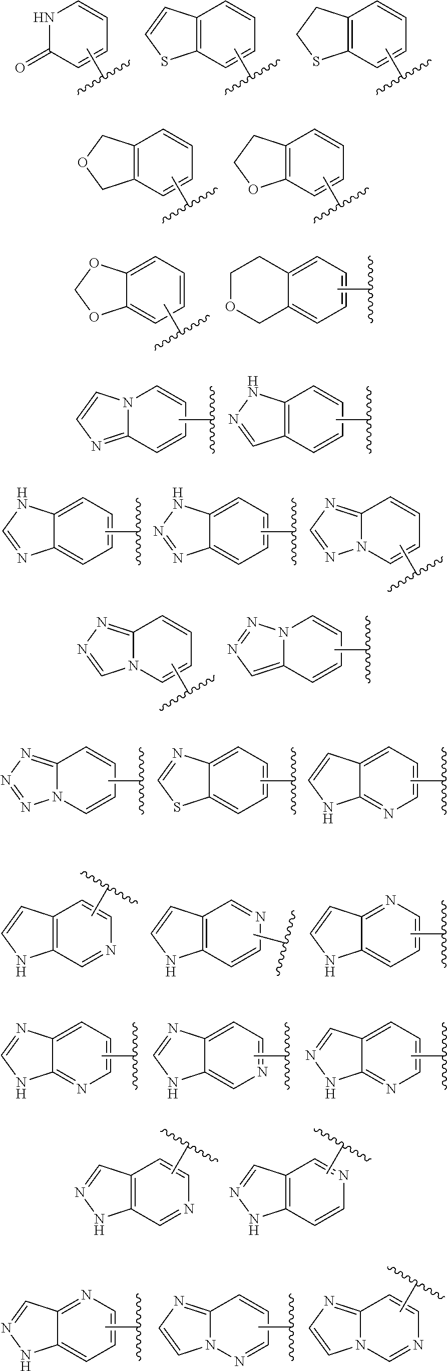

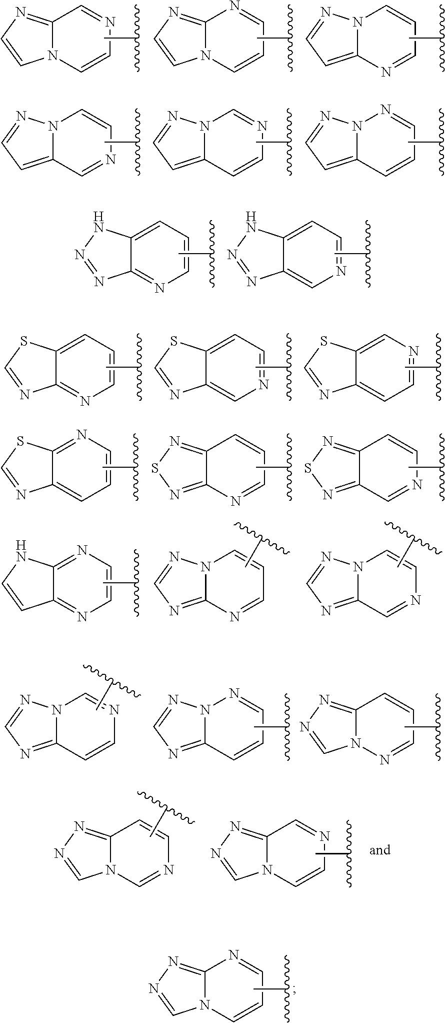

wherein Ring A is optionally substituted with one or more R⁴.
71. Ring A is selected from:

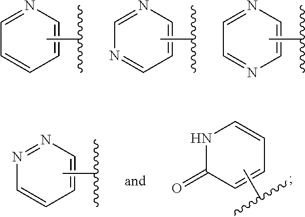

wherein Ring A is optionally substituted with one or more R⁴.
72. Ring A is a 9-membered fused bicyclic heteroaryl containing at least one ring nitrogen atom, wherein Ring A is substituted by one or more R⁴. Thus it may be that Ring A selected from:

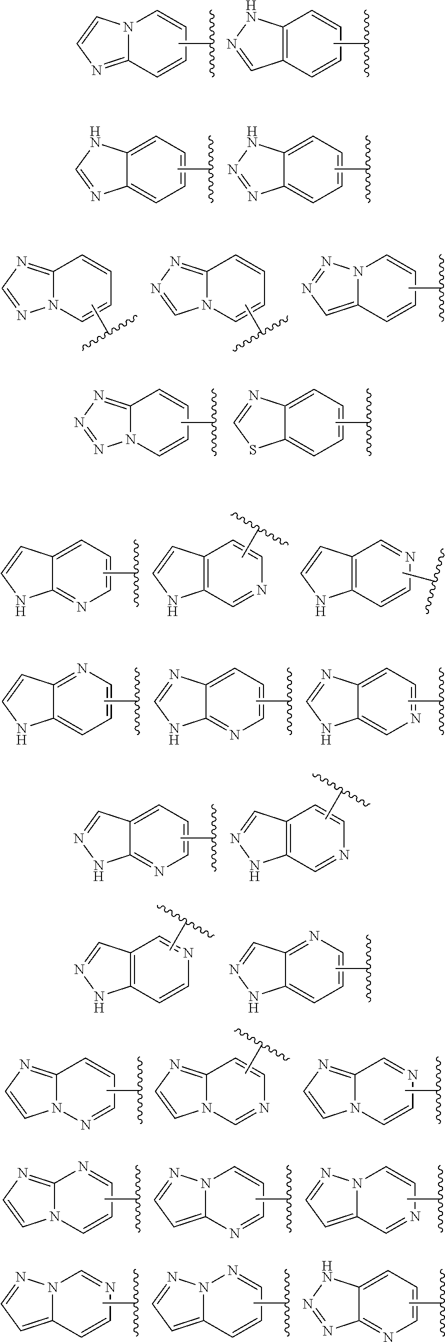

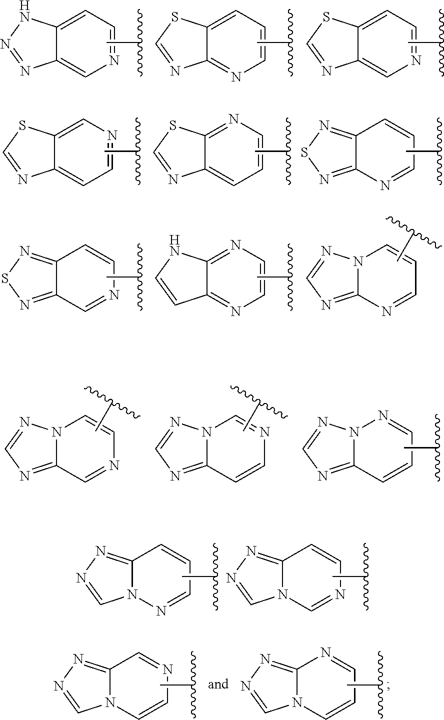

wherein Ring A is optionally substituted with one or more R⁴. It may be that Ring A is attached to L via an atom in a 5-membered ring in Ring A. It may be that Ring A is attached to L via an atom in a 6-membered ring in Ring A.
73. Ring A is selected from:

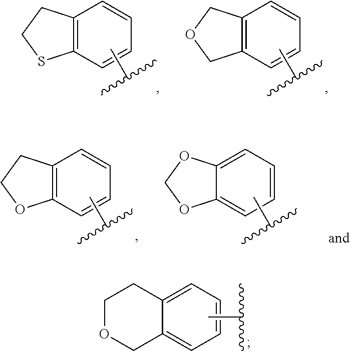

wherein Ring A is optionally substituted with one or more R⁴. It may be that Ring A is attached to L via a carbon atom in a benzo ring. in Ring A.
74. Ring A is selected from:

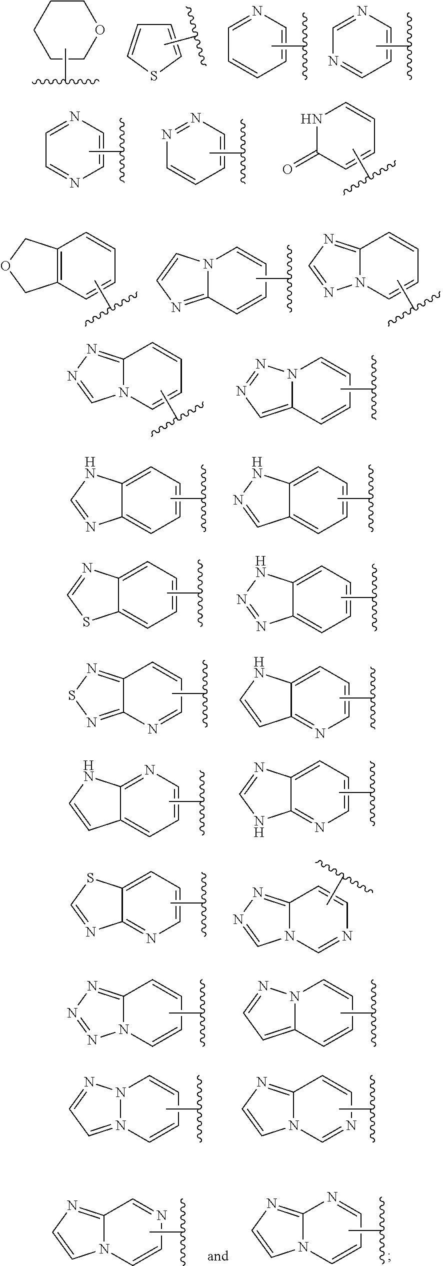

wherein Ring A is optionally substituted with one or more R⁴.
75. Ring A is selected from:

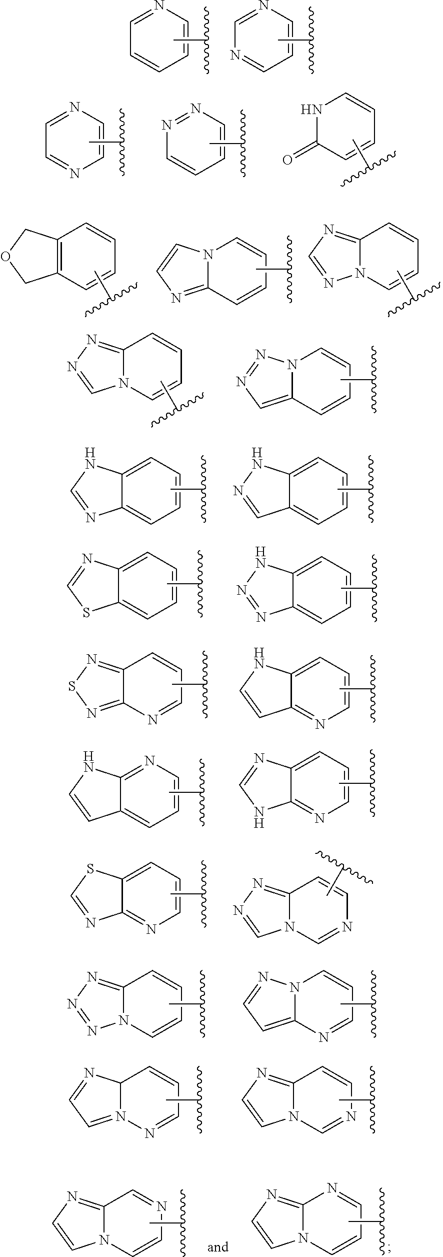

wherein Ring A is optionally substituted with one or more R⁴
76. Ring A is selected from:

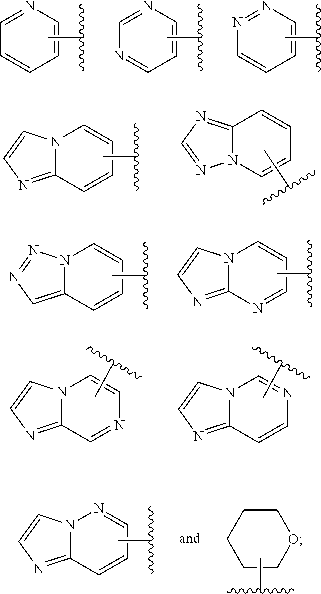

wherein Ring A is optionally substituted with one or more R⁴.
77. Ring A is selected from:

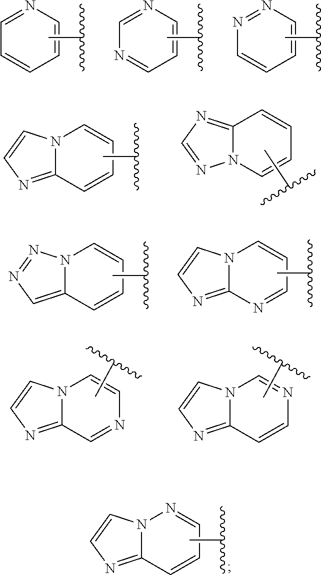

wherein Ring A is optionally substituted with one or more R⁴.
78. Ring A is selected from:

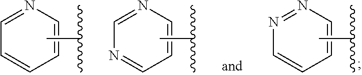

wherein Ring A is optionally substituted with one or more R⁴.
79. Ring A is selected from:

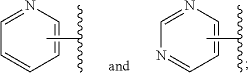

wherein Ring A is optionally substituted with one or more R⁴.
80. Ring A is selected from:

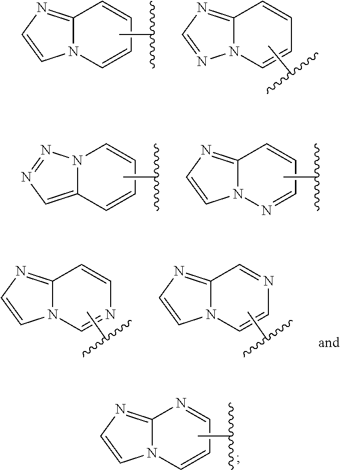

wherein Ring A is optionally substituted with one or more R⁴.
81. Ring A is selected from thienyl and thiazolyl, wherein Ring A is optionally substituted with one or more R⁴.
82. Ring A is selected from:

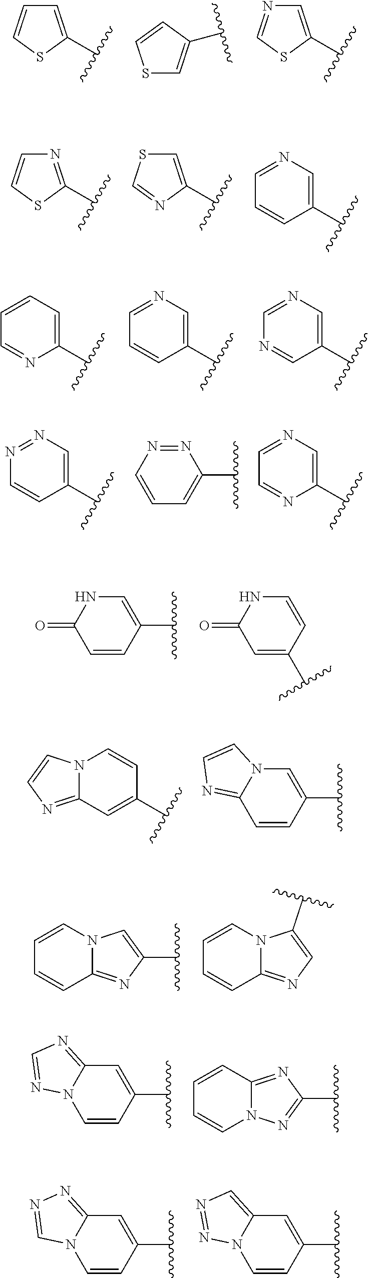

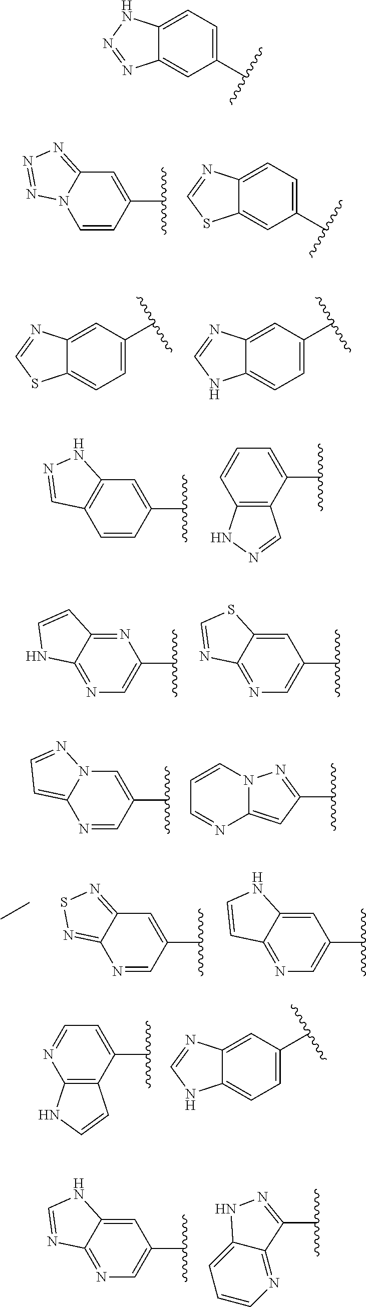

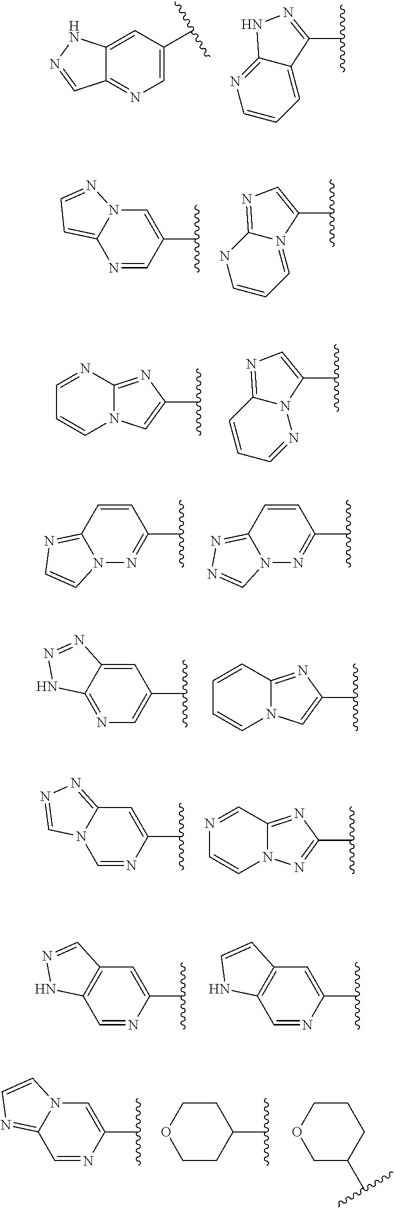

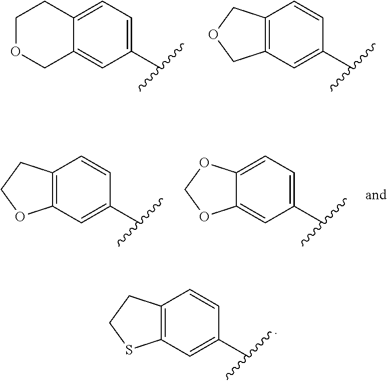 [SMILES: *c1ccc2c(c1)COCC2 ; *c1ccc2c(c1)COC2 ; *c1ccc2c(c1)OCC2 ; *c1ccc2c(c1)OCO2 ; *c1ccc2c(c1)SCC2]

wherein Ring A is optionally substituted with one or more R⁴.
83. Ring A is selected from:

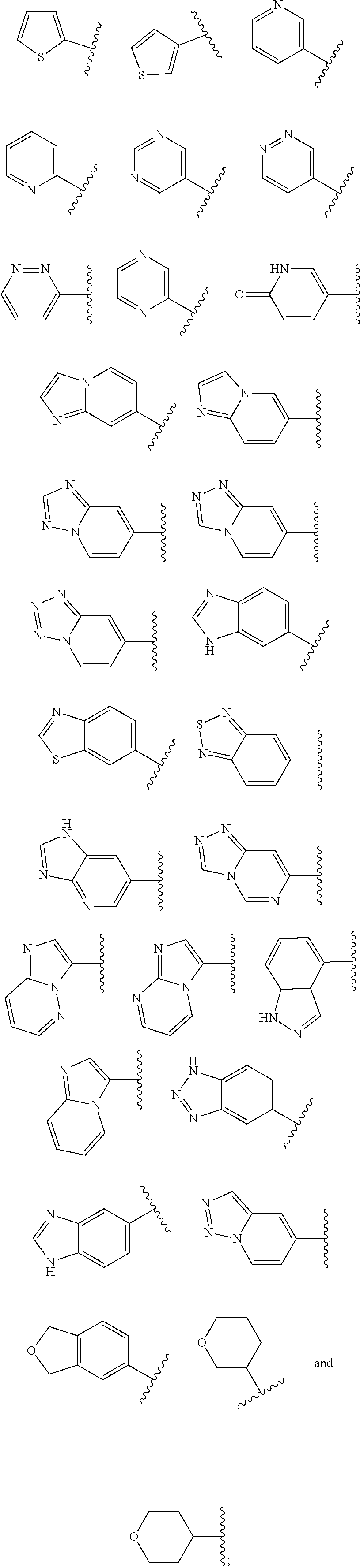

wherein Ring A is optionally substituted with one or more R⁴.
84. Ring A is selected from:

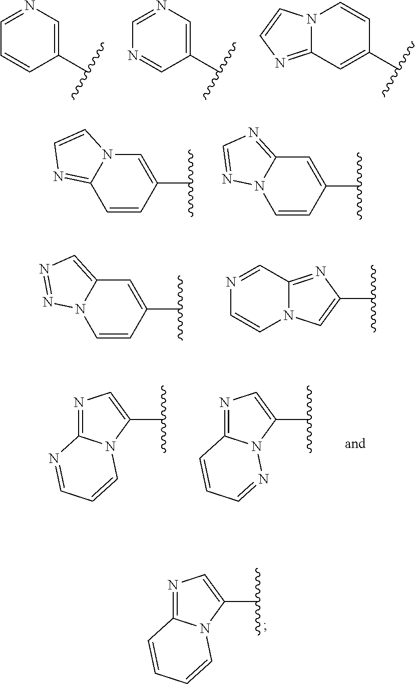

wherein Ring A is optionally substituted with one or more R⁴.
85. Ring A is selected from:

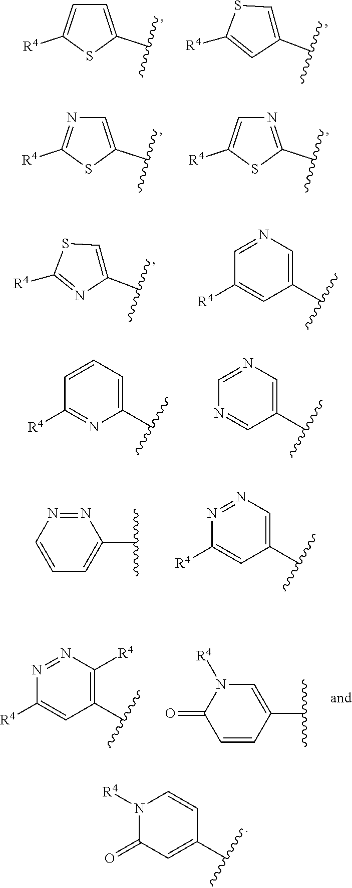

86. Ring A is selected from:

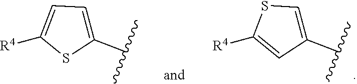

87. Ring A is selected from:

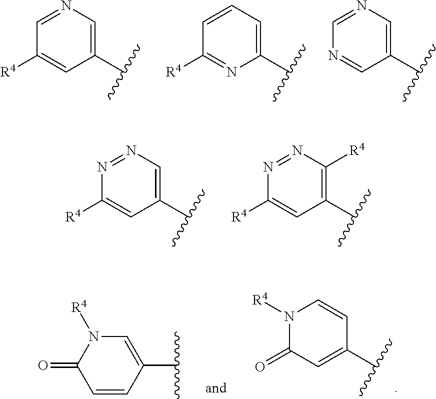

Thus it may be that Ring A is selected from:

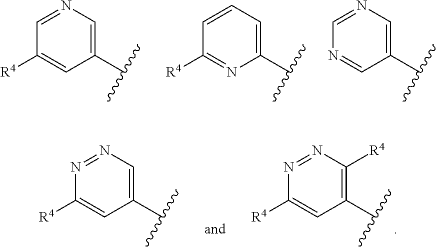

88. Ring A is selected from:

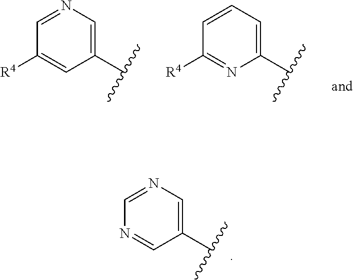

89. Ring A is selected from:

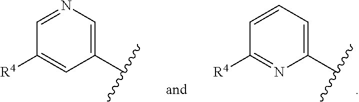

90. Ring A is selected from:

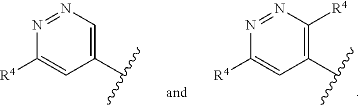

91. Ring A is as defined in any of 40 to 84 and is substituted by one or more R⁴.
92. Ring A is as defined in any of 40 to 84 and is substituted by one or two R⁴.
93. Ring A is as defined in any of 40 to 84 and is unsubstituted.
94. Ring A is selected from:

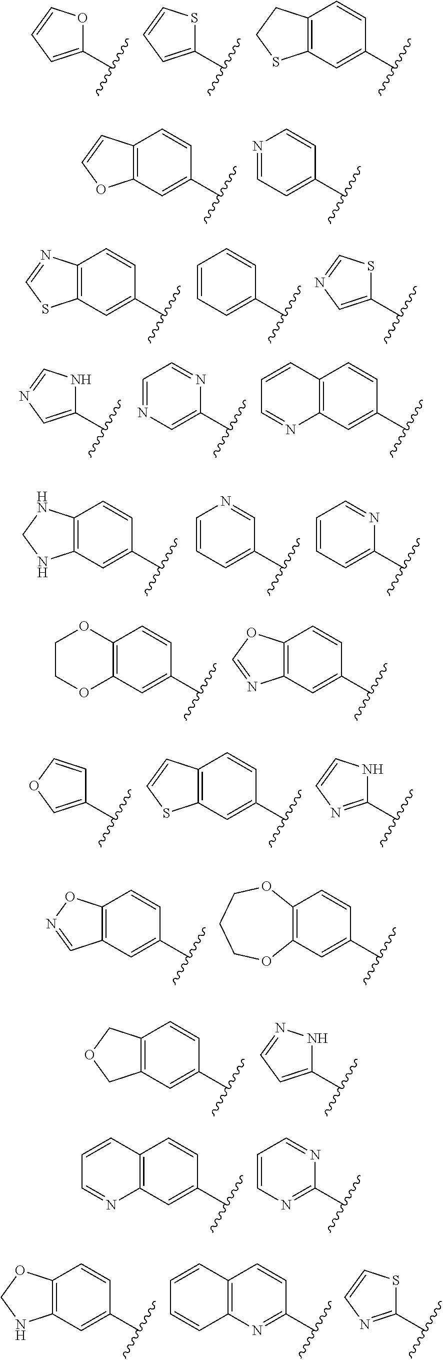

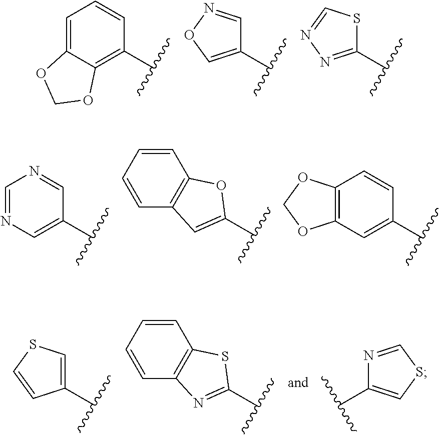

wherein Ring A is optionally substituted with one or more R⁴.
95. Ring A is selected from:

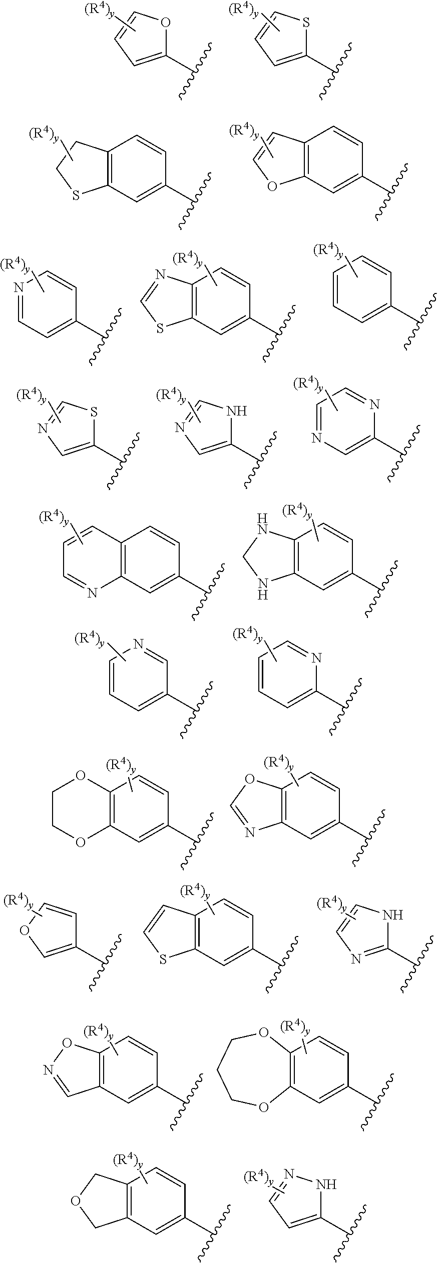

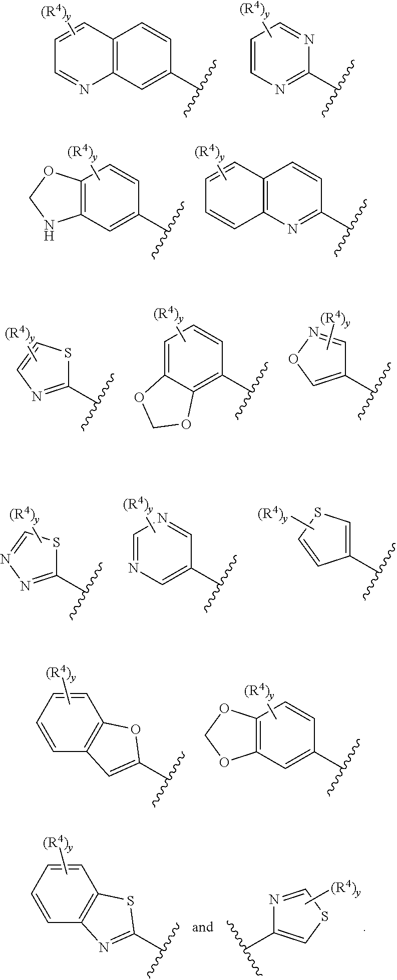

96. Ring A is selected from:

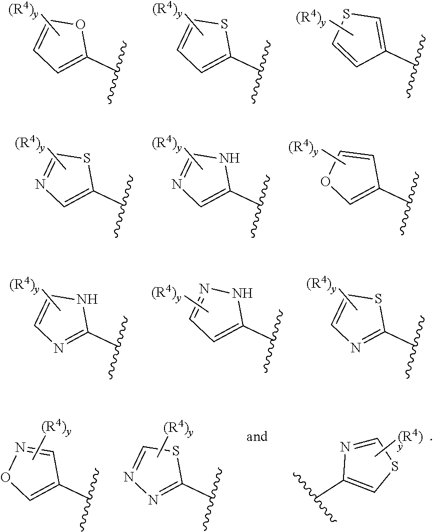

97. Ring A is selected from:

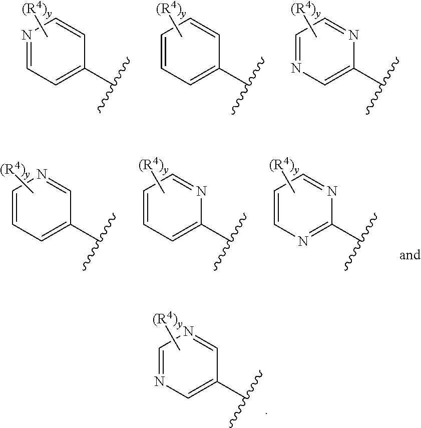

98. Ring A is selected from:

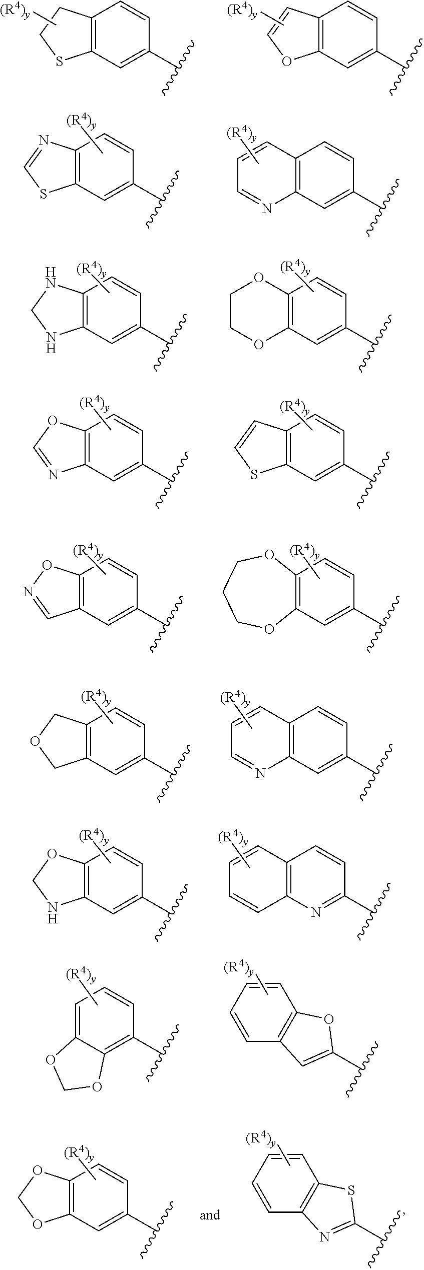

99. Ring A is as defined in any of 95 to 98, wherein y is an integer from 0 to 10, where chemically possible.
100. Ring A is as defined in any of 95 to 98, wherein y is an integer from 0 to 5, where chemically possible.
101. Ring A is as defined in any of 95 to 98, wherein y is an integer from 0 to 3, where chemically possible.
102. Ring A is as defined in any of 95 to 98, wherein y is 0.
103. Ring A is as defined in any of 95 to 98, wherein y is 1.
104. Ring A is as defined in any of 95 to 98, wherein y is 2.
105. Where the Ring A defined in any of 40 to 104 comprises an NH group, said NH group may be substituted by R⁴ to give NR⁴.
106. Each R⁴ is independently selected from: halo, —CN, —NO₂, ═O, C₁₋₆ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵SO₂R⁶, and —SO₂NR⁵R⁶.
107. Each R⁴ is independently selected from: halo, —CN, —NO₂, C₁₋₆ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵SO₂R⁶, and —SO₂NR⁵R⁶. Thus it may be that each R⁴ is independently selected from: halo, —CN, —NO₂, C₁₋₆ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —C(O)NR⁵R⁶, —NR⁵SO₂R⁶, and —SO₂NR⁵R⁶.
108. Each R⁴ is independently selected from: halo, —CN, ═O, C₁₋₆ alkyl, C₁₋₆ haloalkyl, Q¹, —OR⁵, —NR⁵R⁶, —C(O)R⁵, —C(O)OR⁵, and —C(O)NR⁵R⁶.
109. Each R⁴ is independently selected from: halo, —CN, C₁₋₄ alkyl, C₁₋₄ haloalkyl, —OR⁵, —NR⁵R⁶, —C(O)R⁵, —C(O)OR⁵, and —C(O)NR⁵R⁶.
110. Each R⁴ is independently selected from: halo, —CN, ═O, C₁₋₆ alkyl, 2 to 8 membered heteroalkyl, Q¹, —OR⁵, —NR⁵R⁶, —C(O)R⁵, —C(O)NR⁵R⁶ and —NR⁵C(O)R⁶
111. Each R⁴ is independently selected from: halo, —CN, C₁₋₄ alkyl, —OR⁵, —NR⁵R⁶, —C(O)R⁵, —C(O)NR⁵R⁶ and —NR⁵C(O)R⁶.
112. Each R⁴ is independently selected from: halo, —CN, C₁₋₄ alkyl, —C(O)R⁵ and —C(O)NR⁵R⁶.
113. Each R⁴ is independently selected from halo (e.g. fluoro or chloro), —CN, C₁₋₃ alkyl, —OC₁₋₃ alkyl, —C(O) C₁₋₃ alkyl, —C(O)NH₂, —C(O)NH(C₁₋₃ alkyl) and —C(O)N(C₁₋₃ alkyl)₂.
114. Each R⁴ is independently selected from halo (e.g. fluoro or chloro), —CN, C₁₋₃ alkyl and —OC₁₋₃ alkyl. Thus it may be that each R⁴ is independently selected from halo, —CN and C₁₋₃ alkyl. Thus it may be that each R⁴ is independently selected from fluoro, chloro, —CN, methyl and methoxy. For example each R⁴ is independently selected from fluoro, chloro, —CN and methyl.
115. R⁴ is as defined in any one of 106 to 111, wherein said C₁₋₆ alkyl or 2 to 8 membered heteroalkyl is substituted by one or more R⁷.
116. R⁵ and R⁶ are each independently selected from: H, C₁₋₆ alkyl, and Q¹.
117. R⁵ and R⁶ are each independently selected from: H, C₁₋₃ alkyl and Q¹.
118. R⁵ and R⁶ are each independently selected from: H and C₁₋₃ alkyl.
119. R⁵ and R⁶ are as defined in 116 to 118, wherein said alkyl is substituted by one or more R⁸.
120. R⁷ and R⁸ are each independently selected from: —C(O)R^{7A}, —OC(O)R^{7A}, —C(O)OR^{7A}, —NR^{7A}C(O)R^{7B} and —C(O)NR^{7A}R^{7B}.
121. R⁷ and R⁸ are each independently selected from: halo, —CN, —OR^{7A}, —NR^{7A}R^{7B} and Q².
122. R⁷ and R⁸ are each independently selected from: halo, —OR^{7A} and Q².
123. Q¹ and Q² are each independently selected from: C₃₋₆ cycloalkyl and 4- to 7-membered heterocyclyl.
124. Q¹ and Q² are each independently 4- to 6-membered heterocyclyl.
125. Q¹ and Q² are each independently selected from: oxetanyl, azetidinyl, tetrahydrofuranyl, pyrrolidinyl, piperidinyl, morpholinyl, piperazinyl, tetrahydropyranyl and dihydropyranyl.
126. Q¹ and Q² are each independently selected from: phenyl and 5- or 6-membered heteroaryl.
127. Q¹ and Q² are as defined in any of 123 or 126, wherein Q¹ and Q² are substituted by one or more R⁹.
128. Each R⁹ is independently selected from: halo, ═O, —CN, —NO₂, C₁₋₄ alkyl, C₁₋₄ haloalkyl, —OR^{9A}, and —NR^{9A}R^{9B}.
129. R⁹ is as defined in 128, wherein said C₁₋₄ alkyl is substituted by 1 or 2 substituents selected from: halo, —CN, —OR^{9C}, —NR^{9C}R^{9D} and —SO₂R^{9C}.
130. R^{7A}, R^{7B}, R^{9A}, R^{9B}, R^{9C}, and R^{9D} are at each occurrence independently selected from: H, and C₁₋₄ alkyl.
131. R^{7A}, R^{7B}, R^{9A}, R^{9B}, R^{9C}, and R^{9D} are at each occurrence independently selected from: H, methyl, and ethyl.
132. R^{7A}, R^{7B}, R^{9A}, R^{9B}, R^{9C}, and R^{9D} are at each occurrence independently selected from: H and methyl.
133. Each R⁴ is independently selected from:

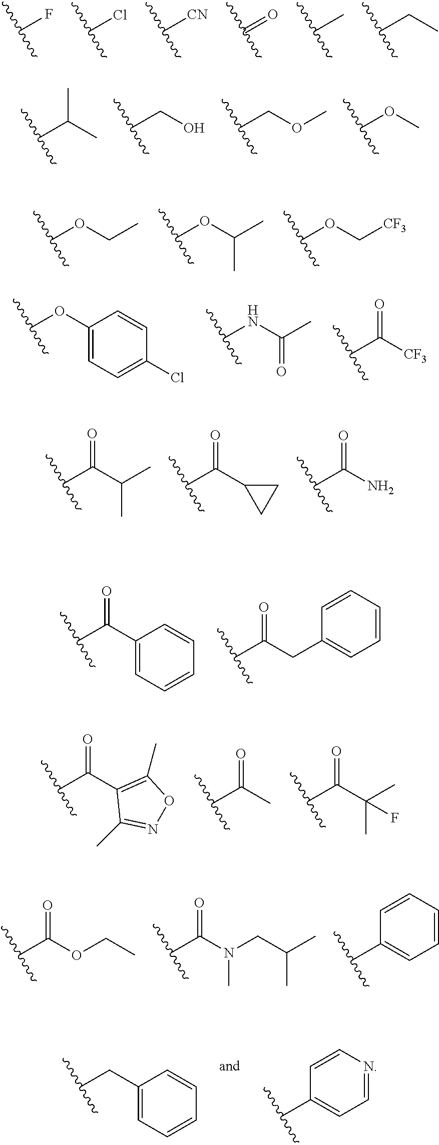

134. Each R⁴ or R^{4a} is independently selected from: F, Cl, —CN, methyl, methoxy, —NH₂, —NH(Me), —NH (Et), —N(Me)₂-C(O)Me, —C(O)NH₂, —C(O)NH(Me), —C(O)N(Me)₂, —C(O)OMe, oxetanyl, azetidinyl, tetrahydrofuranyl, pyrrolidinyl, piperidinyl, morpholinyl, piperazinyl, tetrahydropyranyl and dihydropyranyl.
135. Each R⁴ or R^{4a} is independently selected from: F, Cl, —CN, methyl, methoxy, —NH₂, —NH(Me), —NH (Et), —N(Me)₂-C(O)Me, —C(O)NH₂, —C(O)NH(Me), —C(O)N(Me)₂ and —C(O)OMe.
136. Each R⁴ or R^{4a} is independently selected from: F, —CN, methyl, —C(O)Me, —C(O)NH₂, —C(O)NH(Me) and —C(O)N(Me)₂.
137. Each R⁴ or R^{4a} is independently selected from: halo (e.g. fluoro), —CN and C₁₋₃ alkyl (e.g. methyl). This it may be that each R⁴ is independently selected from: fluoro, —CN and methyl.
138. Ring A is selected from:

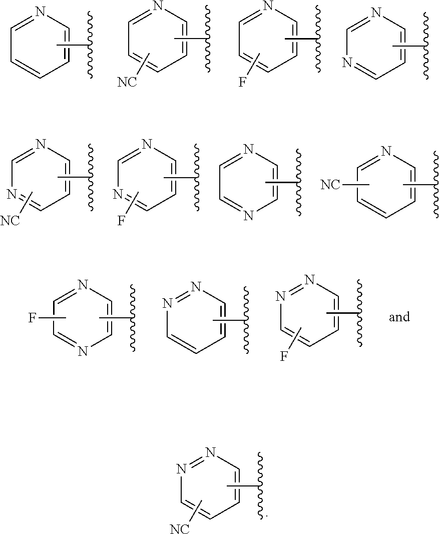

139. Ring A is selected from:

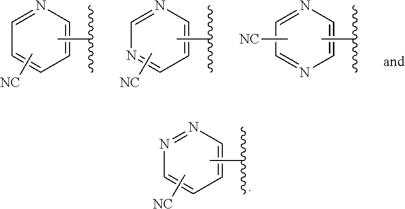

140. Ring A is selected from:

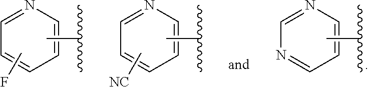

141. Ring A is selected from:

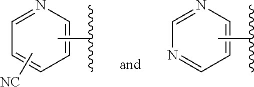

142. Ring A is selected from:

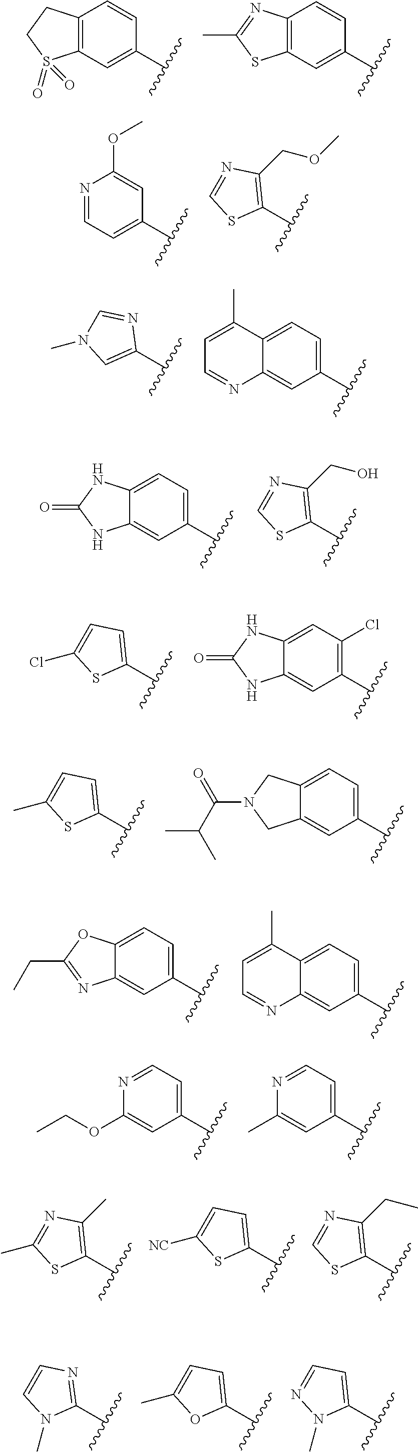

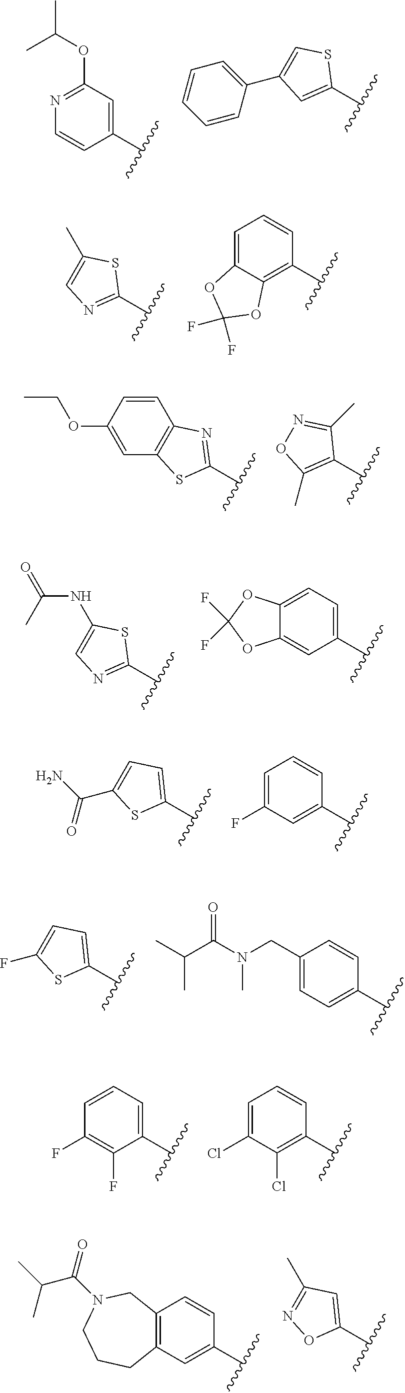

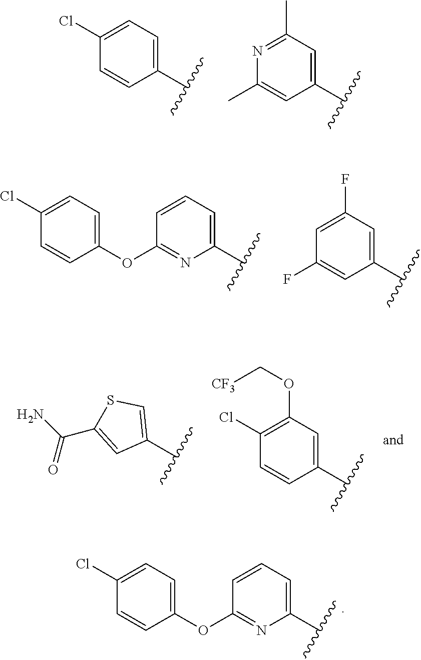

143. Ring A is selected from:

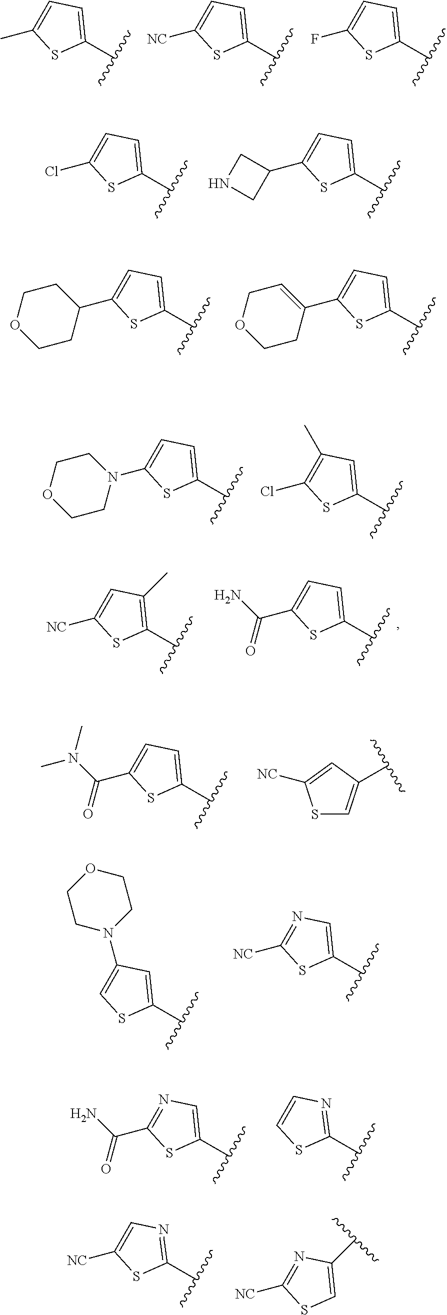

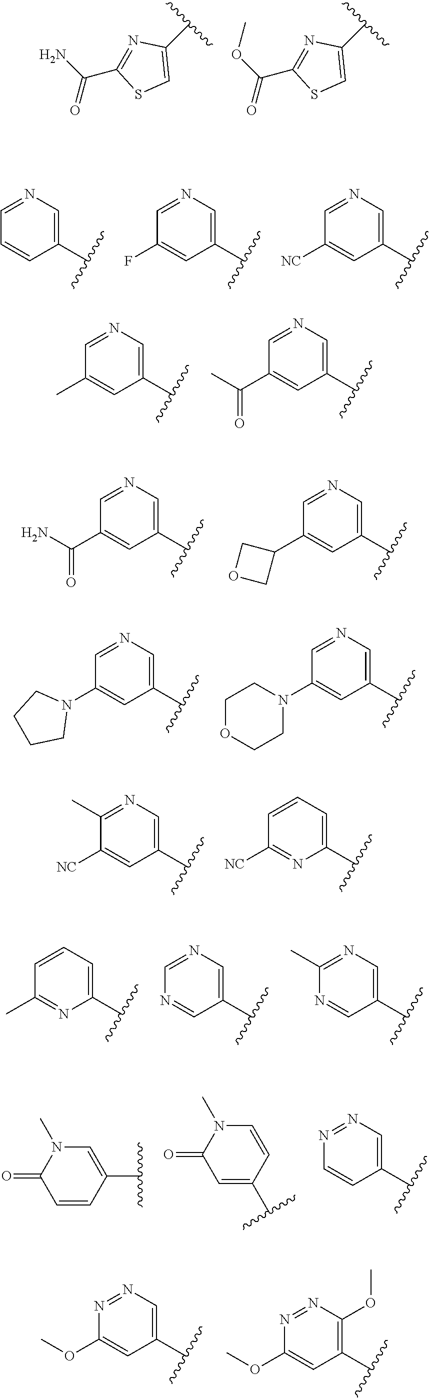

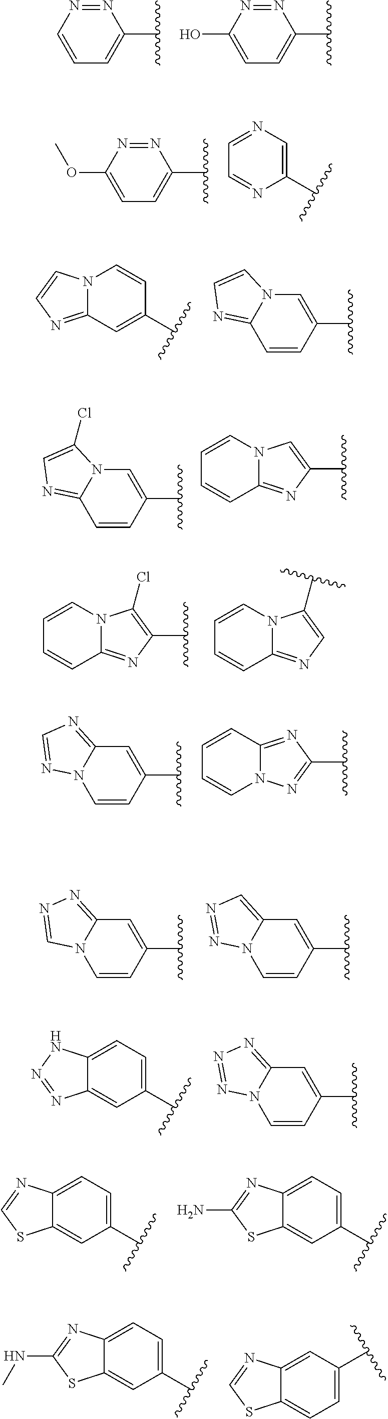

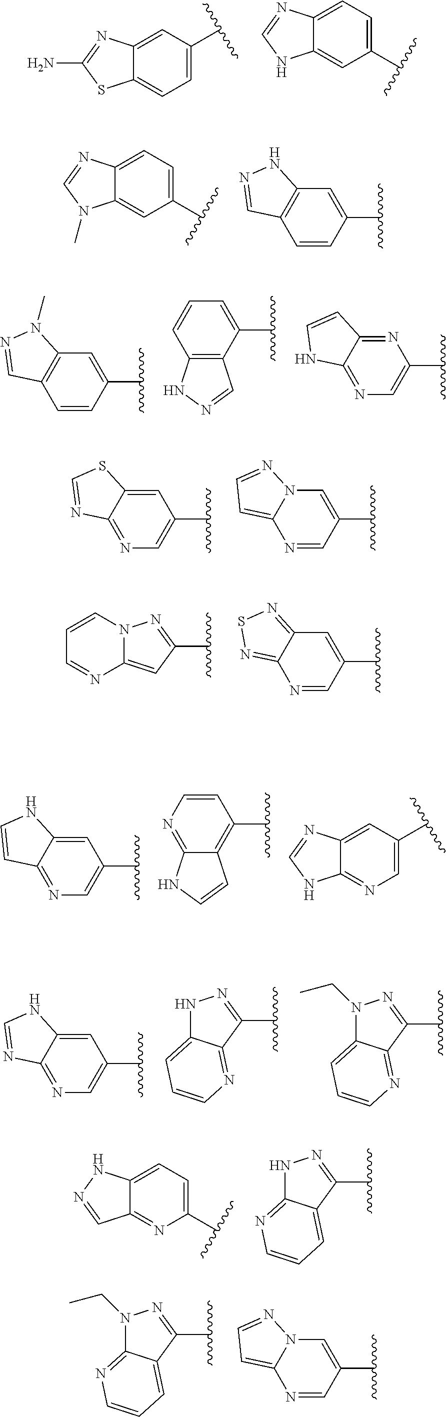

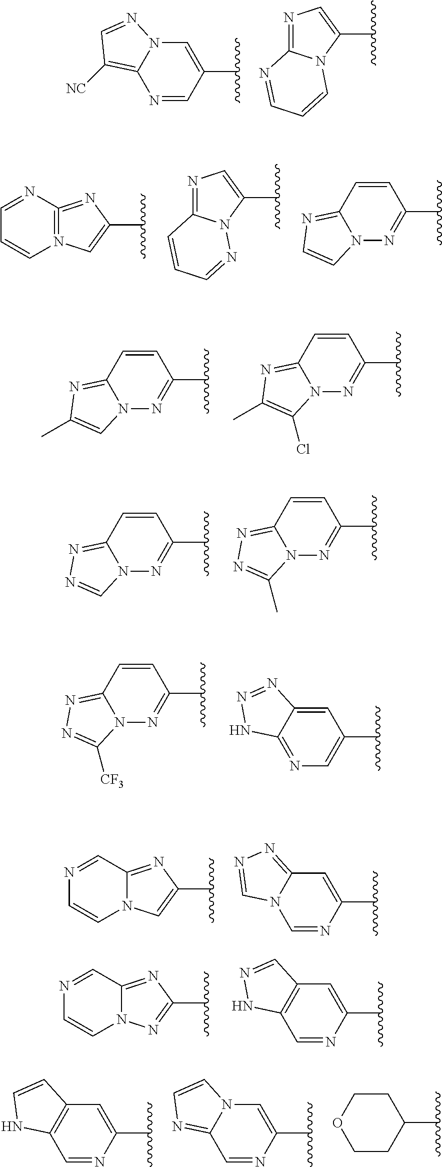

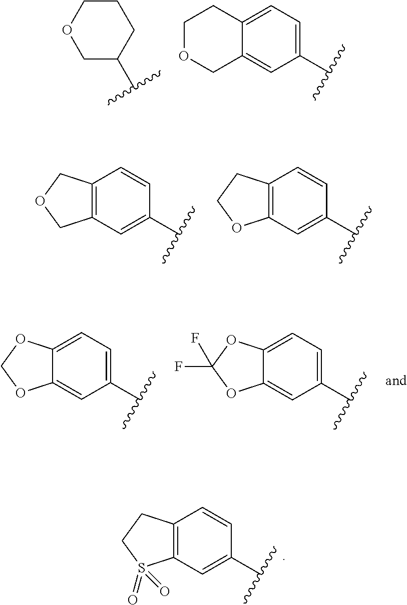

144. Ring A is selected from:

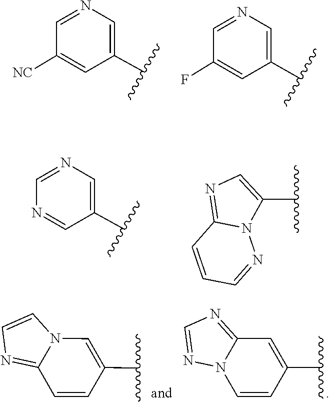

Thus it may be that Ring A is

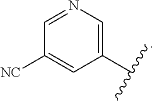 [SMILES: *c1cncc(C#N)c1]

It may be that Ring A is

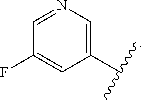 [SMILES: *c1cncc(F)c1]

It may be that Ring A is

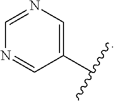 [SMILES: *c1cncnc1]

It may be that Ring A is

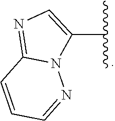 [SMILES: *c1cnc2cccnn12]

It may be that Ring A is

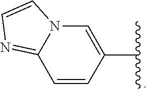 [SMILES: *c1ccc2nccn2c1]

It may be that Ring A is

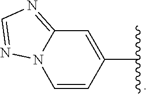 [SMILES: *c1ccn2ncnc2c1]

145. a is an integer from 0 to 5.
146. a is an integer from 0 to 3.
147. a is 3.
148. a is 2.
149. a is 1.
150. a is 0.
151. x is 0.
152. x is 1.
153. x is 2.
154. R^{4a} and R^{4b} are each independently selected from: halo, —CN, —NO₂, ═O, C₁₋₆ alkyl, C₁₋₈ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵SO₂R⁸, and —SO₂NR⁵R⁶. Thus it may be that R^{4a} and R^{4b} are each independently selected from: halo, —CN, —NO₂, C₁₋₆ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —C(O)NR⁵R⁶, —NR⁵SO₂R⁶, and —SO₂NR⁵R⁶.
155. Each R^{4a} and R^{4b} is independently selected from: halo, —CN, —NO₂, C₁₋₆ alkyl, C₁₋₆ haloalkyl, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵SO₂R⁶, and —SO₂NR⁵R⁶.
156. Each R^{4a} and R^{4b} is independently selected from: halo, —CN, ═O, C₁₋₆ alkyl, C₁₋₆ haloalkyl, Q¹, —OR⁵, —NR⁵R⁶, —C(O)R⁵, —C(O)OR⁵, and —C(O)NR⁵R⁶.
157. Each R^{4a} and R^{4b} is independently selected from: halo, —CN, C₁₋₆ alkyl, C₁₋₆ haloalkyl, Q¹, —OR⁵, —NR⁵R⁶, —C(O)R⁵, —C(O)OR⁵, and —C(O)NR⁵R⁶.
158. Each R^{4a} and R^{4b} are each independently selected from: halo, —CN, ═O, C₁₋₆ alkyl, Q¹, —OR⁵, —NR⁵R⁶, —C(O)R⁵, and —NR⁵C(O)R⁶.
159. Each R^{4a} and R^{4b} is selected from: halo (e.g. fluoro or chloro), —CN, C₁₋₃ alkyl, —OC₁₋₃ alkyl, —C(O) C₁₋₃ alkyl, —C(O)NH₂, —C(O)NH(C₁₋₃ alkyl) and —C(O)N(C₁₋₃ alkyl)₂.
160. Each R^{4a} and R^{4b} is selected from: halo (e.g. fluoro), —CN and C₁₋₃ alkyl (e.g. methyl).
161. R^{4a} and R^{4b} are each as defined in any one of 154 to 158, wherein said C₁₋₆ alkyl is substituted by one or more R⁷.
162. Ring B is selected from phenyl or 6-membered heteroaryl.
163. Ring B is 6-membered heteroaryl.
164. Ring B is 5-membered heteroaryl.
165. Ring B is selected from furanyl, thienyl furanyl, thienyl, imidazolyl, pyrazolyl, oxazolyl, isoxazolyl, thiazolyl, isothiazolyl, thiadiazolyl, phenyl, pyridyl, pyrimidinyl, and pyrazinyl.
166. Ring B is selected from furanyl, thienyl furanyl, thienyl, imidazolyl, pyrazolyl, oxazolyl, isoxazolyl, thiazolyl, isothiazolyl, thiadiazolyl, phenyl, pyrimidinyl, and pyrazinyl.
167. Ring B is selected from furanyl, pyrazolyl, oxazolyl, isoxazolyl and phenyl.
168. Ring B is furanyl.
169. Ring B is pyrazolyl.
170. Ring B is oxazolyl.
171. Ring B is isoxazolyl.
172. Ring B is phenyl.
173. Ring B is as defined in any of 162 to 172 and is substituted by one or more R¹⁰.
174. Ring B is selected from:

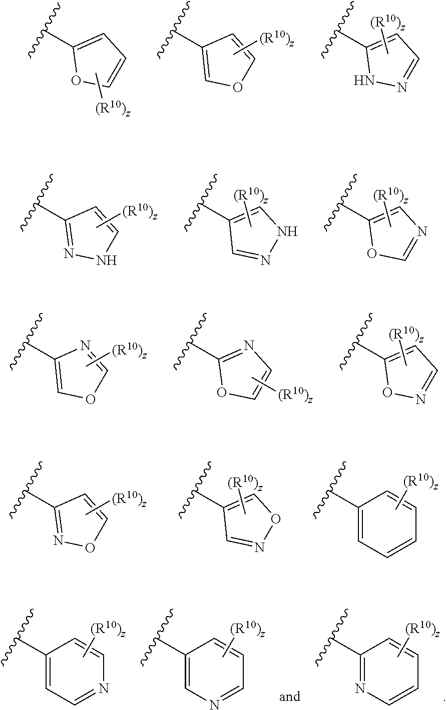

175. Ring B is selected from:

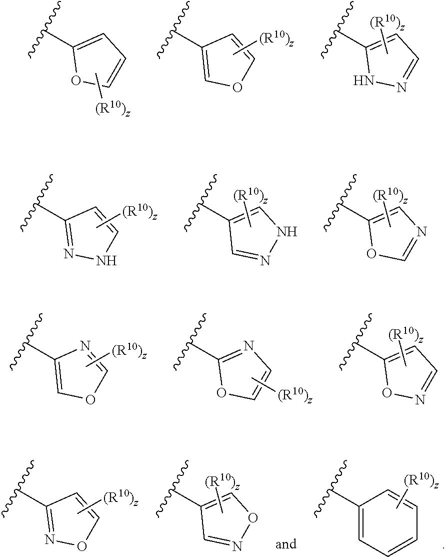

176. Ring B is selected from:

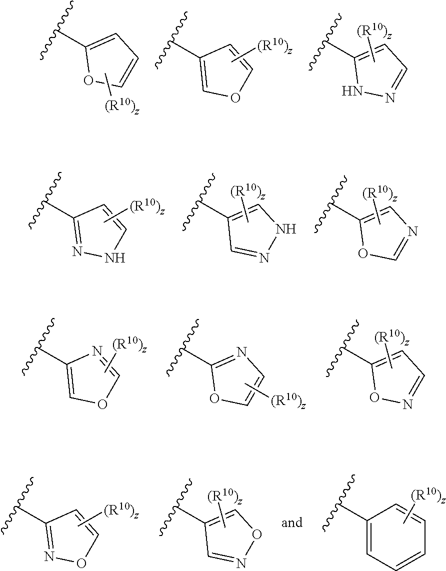

177. Ring B is selected from:

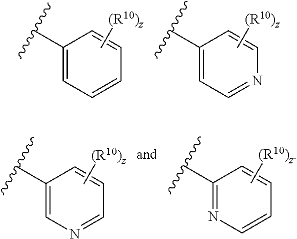

178. Ring B is

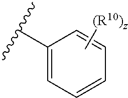

179. Ring B is as defined in any of 174 to 178, wherein z is an integer from 0 to 5, where chemically possible. Thus it may be that Ring B is as defined in any of 174 to 178, wherein z is 0, 1 or 2. It may be that Ring B is as defined in any of 174 to 178, wherein z is 1 or 2.
180. Ring B is as defined in any of 174 to 178, wherein z is 2.
181. Ring B is as defined in any of 174 to 178, wherein z is 1.
182. Ring B is as defined in any of 174 to 178, wherein z is 0.
183. Ring B is selected from:

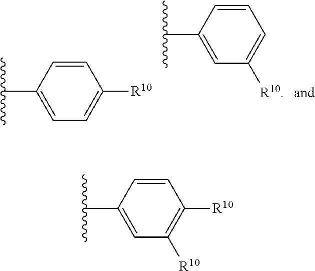

184. Ring B is selected from:

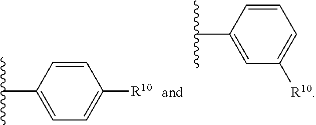

It may be that Ring B is

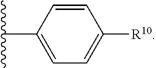

185. Ring B is phenyl substituted by one or two R¹⁰ selected from halo and C₁₋₃ haloalkyl. Thus it may be that Ring B is 4-fluorophenyl or 4-trifluoromethylphenyl. It may be that Ring B is 4-fluorophenyl. It may be that Ring B is 4-trifluoromethylphenyl.
186. Ring B is selected from:

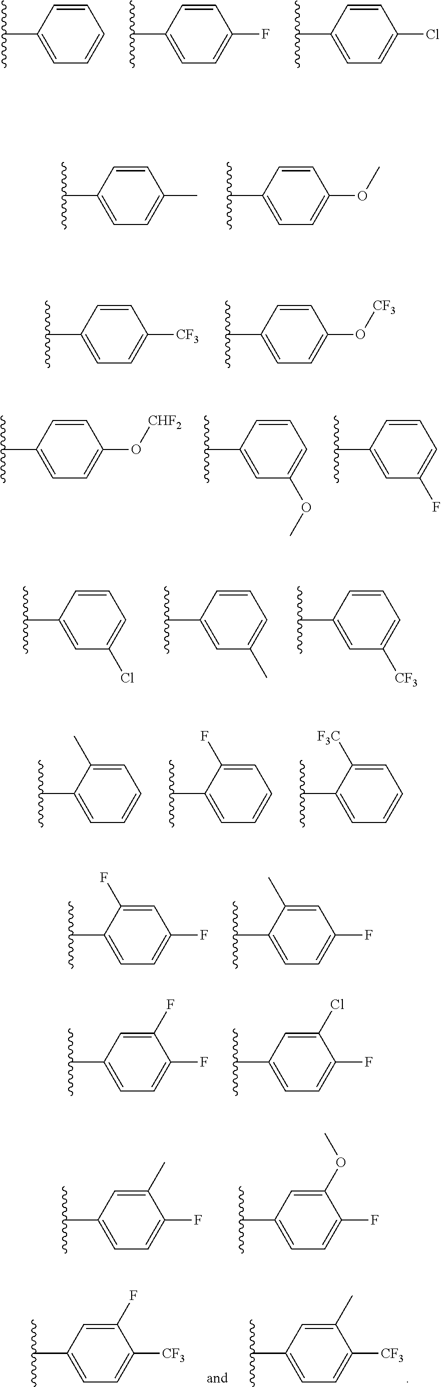

187. Ring B is selected from:

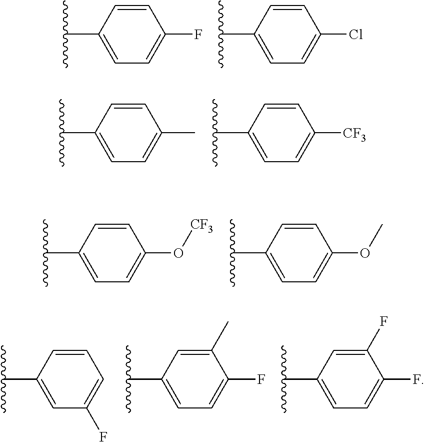

188. Ring B is selected from:

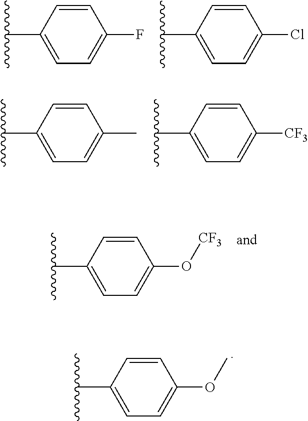

Thus it may be that Ring B is selected from:

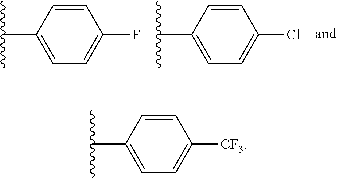

189. Where the Ring B defined in any of 162 to 176 comprises an NH group, said NH group may be substituted by R¹⁰ to give NR¹⁰.
190. Each R¹⁰ and R^{10a} is independently selected from: halo, —CN, —NO₂, C₁₋₄ alkyl, C₁₋₄ haloalkyl, —OR^{10A}, —S(O)ₓR^{10A}, and —NR^{10A}R^{10B}.
191. Each R¹⁰ and R^{10a} is independently selected from —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
192. Each R¹⁰ and R^{10a} is independently selected from: halo, —CN, —NO₂, C₁₋₆ alkyl, C₁₋₆ haloalkyl, —OR^{10A}, and —S(O)ₓR^{10A}.
193. Each R¹⁰ and R^{10a} is independently selected from: halo, —CN, —NO₂, C₁₋₃ alkyl, C₁₋₃ haloalkyl, —OR^{10A}, and —S(O)ₓR^{10A}
194. Each R¹⁰ and R^{10a} is independently selected from: halo, C₁₋₃ alkyl, C₁₋₃ haloalkyl, —OC₁₋₃ alkyl and —OC₁₋₃ haloalkyl.
195. Each R¹⁰ and R^{10a} is independently selected from: halo and C₁₋₃ alkyl.
196. Each R¹⁰ and R^{10a} is independently selected from: halo, —CN, —NO₂, methyl, CF₃, —OH, —OMe, and —S(O)₂Me.
197. Each R¹⁰ and R^{10a} is independently selected from: fluoro, chloro, —CN, —NO₂, methyl, —CF₃, —OH, —OMe, and —S(O)₂Me.
198. Each R¹⁰ and R^{10a} is independently selected from: fluoro, chloro, methyl, —CF₃, methoxy, —OCF₃ and —OCHF₂.
199. Each R¹⁰ and R^{10a} is independently selected from: fluoro and methyl.
200. R¹⁰ and R^{10a} are fluoro.
201. R¹⁰ and R^{10a} are —CF₃.
202. R¹⁰ and R^{10a} are as defined in any of 190 and 192 to 195, wherein said alkyl is substituted by one or more R¹¹.
203. Each R¹¹ is independently selected from: halo, —CN, —OR^{11A}, —NR^{11A}R^{11B} and —SO₂R^{11A}.
204. Each R¹¹ is independently selected from: halo, —CN, —OR^{11A}, and —NR^{11A}R^{11B}
205. Each R¹¹ is independently selected from: halo and —OR^{11A}.
206. R^{10A}, R^{10B}, R^{11A} and R^{11B} are at each occurrence independently selected from: H, and C₁₋₄ alkyl.
207. R^{10A}, R^{10B}, R^{11A} and R^{11B} are at each occurrence independently selected from: H, methyl, and ethyl.
208. R^{10A}, R^{10B}, R^{11A} and R^{11B} are at each occurrence independently selected from: H and methyl.
209. c is an integer from 0 to 5.
210. c is an integer from 0 to 4.
211. c is an integer from 0 to 3.
212. c is 3.
213. c is 2.
214. c is 1.
215. c is 0.
216. Any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 4-membered heterocyclyl.
217. Any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 5-membered heterocyclyl.
218. Any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 6-membered heterocyclyl.
219. Any 4- to 6-membered heterocyclyl defined in any of 216 to 218 is substituted by one or more substituents selected from: halo, ═O, C₁₋₄ alkyl and C₁₋₄ haloalkyl.
220. Any 4- to 6-membered heterocyclyl defined in any of 216 to 218 is substituted by one or more substituents selected from: halo, C₁₋₄ alkyl and C₁₋₄ haloalkyl.
221. Any 4- to 6-membered heterocyclyl defined in any of 216 to 218 is substituted by one or more substituents selected from: halo and C₁₋₄ alkyl.
222. The group of the formula:

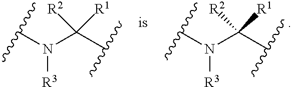

Thus it may be that the group of the formula:

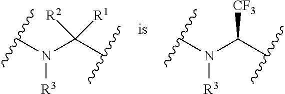

223. The group of the formula:

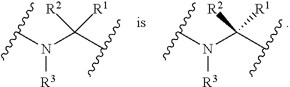

Thus it may be that the group of the formula:

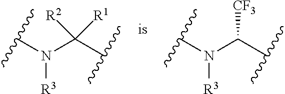

In an embodiment, the compound of formula (I) is a compound of any of formulae (I) to (XXXVIc), wherein L is a bond.

In an embodiment, the compound of formula (I) is a compound of any of formulae (I) to (XXXVIc), wherein L is —CH₂—.

In an embodiment, the compound of formula (I) is a compound of any of formulae (I) to (XXXVIc), wherein L is a bond and R³ is selected from methyl, ethyl, and —CH₂CH₂F.

In an embodiment, the compound of formula (I) is a compound of any of formulae (I) to (XXXVIc), wherein L is a bond and R³ is methyl or ethyl.

In certain embodiments, the compound is a compound according to any of formulae (I), (Ia), (III), (IIIa), (V), (VII), (VIIa), (IX), (IXa), (XI), (XIa), (XIII), (XIIIa), (XV), (XVa), (XVII), (XVIIa), (XIX), (XIXa), (XXI), (XXIa), (XXIII), (XXIIIa), (XXV), (XXVa), (XXVII), (XXVIIa), (XXIX), (XXXI), (XXXII), (XXXIIa), (XXXIII), (XXXIIIa), (XXXIV), (XXXIVa), (XXXV), (XXXVa), (XXXVI) and (XXXVIa), wherein R¹ is selected from —CH₂F, —CHF₂ and —CF₃.

Suitably in this embodiment R² is H. Suitably in this embodiment L is a bond and R³ is selected from methyl, ethyl, and —CH₂CH₂F.

In certain embodiments in any of the compounds of formulae (I), (Ia), (II), (IIa), (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX), (XXa), (XXI), (XXIa), (XXII), (XXIIa), (XXIII), (XXIIIa), (XXIV) and (XXIVa) L is a bond; R² (when present) is H; R³ is selected from methyl, ethyl and —CH₂CH₂F; and Ring B is as defined in any of 162 to 188.

In these embodiments it may be that R³ is methyl or ethyl.

In these embodiments it may be that R³ is methyl. In these embodiments it may be that R³ is ethyl. In these embodiments it may be that R³ is —CH₂CH₂F.

In these embodiments it may be that Ring B is unsubstituted or is substituted by one or two R¹⁰ (or R^{10a} as appropriate for each formula), wherein each R¹⁰ and R^{10a} is independently as defined in any of 190 to 202. Thus it may be that Ring B is unsubstituted or is substituted by one or two R¹⁰ (or R^{10a} as appropriate for each formula), wherein each R¹⁰ and R^{10a} is independently selected from: halo, C₁₋₃ alkyl, C₁₋₃ haloalkyl, —OC₁₋₃ alkyl and —OC₁₋₃ haloalkyl.

In these embodiments it may be that Ring B is unsubstituted phenyl or phenyl substituted by one or two R¹⁰ (or R^{10a} as appropriate for each formula), wherein each R¹⁰ and R^{10a} is independently as defined in any of 190 to 202. For example each R¹⁰ and R^{10a} is independently selected from: halo, C₁₋₃ alkyl, C₁₋₃ haloalkyl, —OC₁₋₃ alkyl and —OC₁₋₃ haloalkyl.

In these embodiments it may be that Ring B is phenyl substituted by one or two R¹⁰ (or R^{10a} as appropriate for each formula), wherein each R¹⁰ and R^{10a} is independently as defined in any of 190 to 202; and R³ is methyl or ethyl. For example each R¹⁰ and R^{10a} is independently selected from: halo, C₁₋₃ alkyl, C₁₋₃ haloalkyl, —OC₁₋₃ alkyl and —OC₁₋₃ haloalkyl. Thus it may be that each R¹⁰ and R^{10a} is independently selected from: fluoro, chloro, methyl, —CF₃, methoxy and —OCF₃.

In certain embodiments in any of the compound of any of formulae (I), (Ia), (II), (IIa), (XXV), (XXVa), (XXVI), (XXVIa), (XXVII), (XXVIIa), (XXVIII), (XXVIIIa), (XXIX), (XXIXa), (XXX) and (XXXa): L is a bond; R² (when present) is H; R³ is selected from methyl, ethyl and —CH₂CH₂F; and Ring A is as defined in any of 40 to 98.

In these embodiments it may be that Ring A is optionally substituted by one or more (e.g. 1 or 2) R⁴ (or R^{4a} as appropriate for each formula), wherein each R⁴ or R^{4a} is independently selected from: halo, —CN, C₁₋₆ alkyl, C₁₋₆ haloalkyl, —OR⁵, —NR⁵R⁶, —C(O)R⁵, —C(O)OR⁵, and —C(O)NR⁵R⁶. Thus it may be that Ring A is optionally substituted by one or more (e.g. 1 or 2) R⁴ (or R^{4a} as appropriate for each formula), wherein each R⁴ or R^{4a} is independently selected from: F, Cl, —CN, methyl, methoxy, —NH₂, —NH(Me), —NH (Et), —N(Me)₂-C(O)Me, —C(O)NH₂, —C(O)NH(Me), —C(O)N(Me)₂ and —C(O)OMe. It may be that Ring A is optionally substituted by one or more (e.g. 1 or 2) R⁴ (or R^{4a} as appropriate for each formula), wherein each R⁴ or R^{4a} is independently selected from: F, Cl, —CN, methyl and methoxy.

In these embodiments it may be that R³ is methyl or ethyl. In these embodiments it may be that R³ is methyl. In these embodiments it may be that R³ is ethyl. In these embodiments it may be that R³ is —CH₂CH₂F.

In certain embodiments in any of the compounds of the formulae (I), (Ia), (II), (IIa), (XXV), (XXVa), (XXVI), (XXVIa), (XXVII), (XXVIIa), (XXVIII), (XXVIIIa), (XXIX), (XXIXa), (XXX) and (XXXa): L is a bond; R² (when present) is H; R³ is selected from methyl, ethyl and —CH₂CH₂F; and Ring A is as defined in any of 138 to 144. In these embodiments it may be that R³ is methyl or ethyl. In these embodiments it may be that R³ is methyl. In these embodiments it may be that R³ is ethyl. In these embodiments it may be that R³ is —CH₂CH₂F.

In certain embodiments the compound of any of formulae (I), (Ia), (II), (IIa), (XXV), (XXVa), (XXVI), (XXVIa), (XXVII), (XXVIIa), (XXVIII), (XXVIIIa), (XIX), (XIXa), (XXX) and (XXXa) is a compound wherein Ring A is not a ring according to the structure:

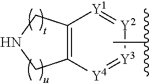

wherein:
Y¹, Y², Y³ and Y⁴ are each independently CH or N, provided no more than two of Y¹, Y², Y³ and Y⁴ are N;
t and u are each independently 0, 1, 2, or 3;
   optionally wherein the ring system is substituted by one or more R⁴, wherein R⁴ is as defined for formula (I).

In certain embodiments the compound of any of formulae (I), (Ia), (II), (IIa), (XXV), (XXVa), (XXVI), (XXVIa), (XXVII), (XXVIIa), (XXVIII), (XXVIIIa), (XIX), (XIXa), (XXX) and (XXXa) is a compound wherein Ring A is not a structure selected from:

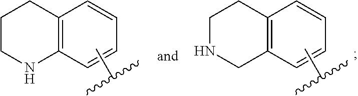

optionally wherein the ring system is substituted by one or more R⁴, wherein R⁴ is as defined for formula (I).

In certain embodiments, the compound is a compound according to any of formulae (I), (Ia), (II), (IIa), (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX), (XXa), (XXI), (XXIa), (XXII), (XXIIa), (XXIII), (XXIIIa), (XXIV) and (XXIVa), wherein Ring B is not pyridyl.

In certain embodiments, the compound is a compound according to any of formulae (I), (Ia), (II), (IIa), (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX), (XXa), (XXI), (XXIa), (XXII), (XXIIa), (XXIII), (XXIIIa), (XXIV) and (XXIVa), wherein Ring B is not

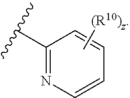

In certain embodiments, the compound is a compound according to any of formulae (I), (Ia), (II), (IIa), (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX), (XXa), (XXI), (XXIa), (XXII), (XXIIa), (XXIII), (XXIIIa), (XXIV) and (XXIVa), wherein Ring B is not

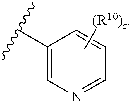

In certain embodiments, the compound is a compound according to any of formulae (I), (Ia), (II), (IIa), (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX), (XXa), (XXI), (XXIa), (XXII), (XXIIa), (XXIII), (XXIIIa), (XXIV) and (XXIVa), wherein Ring B is not

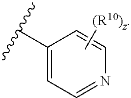

In certain embodiments, the compound is a compound according to any of formulae (III), (IIIa), (IV), (IVa), (V), (Va), (VI), (VIa), (VII), (VIIa), (VIII), (VIIIa), (IX), (IXa) (X), (Xa), (XI), (XIa), (XII), (XIIa), (XIII), (XIIIa), (XIV), (XIVa), (XV), (XVa), (XVI), (XVIa), (XVII), (XVIIa), (XVIII), (XVIIIa), (XIX), (XIXa), (XX) and (XXa), wherein each R^{4a} is independently as defined in any of 154 to 160.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments, the compound is a compound according to any of formulae (XXV), (XXVa), (XXVI), (XXVIa), (XXVII), (XXVIIa), (XXVIII), (XXVIIIa), (XIX), (XIXa), (XXX), (XXXa), (XXXI), (XXXIa), (XXXIb), (XXXIc), (XXXII), (XXXIIa), (XXXIIb) (XXXIIc), (XXXIII), (XXXIIIa), (XXXIIIb), (XXXIIIc) (XXXIV), (XXXIVa), (XXXIVb), (XXXIVc), (XXXV), (XXXVa), (XXXVb), (XXXVc), (XXXVI), (XXXVIa), (XXXVIb) and (XXXVIc), wherein each R^{10a} is independently as defined in any of 190 to 201.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments, the compound is a compound according to any of formulae (XXXI), (XXXIa), (XXXIb), (XXXIc), (XXXII), (XXXIIa), (XXXIIb), (XXXIIc), (XXXV), (XXXVa), (XXXVb), (XXXVc), (XXXVI), (XXXVIa), (XXXVIb) and (XXXVIc), wherein each R^{4a} is independently selected from: halo (e.g. fluoro or chloro), —CN, C₁₋₃ alkyl, —OC₁₋₃ alkyl, —C(O) C₁₋₃ alkyl, —C(O)NH₂, —C(O)NH(C₁₋₃ alkyl) and —C(O)N(C₁₋₃ alkyl)₂; and each R^{10a} is independently selected from: halo, C₁₋₃ alkyl, C₁₋₃ haloalkyl, —OC₁₋₃ alkyl and —OC₁₋₃ haloalkyl.

In this embodiment it may be that each R^{4a} is independently selected from: halo, —CN and C₁₋₃ alkyl, and each R^{10a} is independently selected from: halo, C₁₋₃ alkyl, C₁₋₃ haloalkyl, —OC₁₋₃ alkyl and —OC₁₋₃ haloalkyl.

In this embodiment it maybe that each R^{4a} is independently selected from: fluoro, chloro, —CN, methyl and —OMe; and each R^{10a} is independently selected from: fluoro, chloro, methyl, —CF₃, methoxy, —OCF₃ and —OCHF₂.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments the compound is of the formula (XXXII), (XXXIIa), (XXXIIb) or (XXXIIc), wherein the group of the formula:

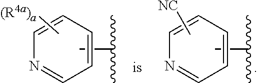

In this embodiment it may be that each R^{10a} is independently as defined in any of 190 to 201.

Suitably in these embodiments each R^{10a} may be independently selected from: fluoro, chloro, methyl, —CF₃, methoxy, —OCF₃ and —OCHF₂.

Suitably in these embodiments L is a bond; R² (when present) is H; and R³ is selected from methyl, ethyl and —CH₂CH₂F.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments the compound is of the formula (XXXII), (XXXIIa), (XXXIIb) or (XXXIIc), wherein the group of the formula:

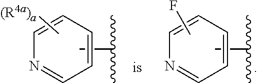

In this embodiment it may be that each R^{10a} is independently as defined in any of 190 to 201.

Suitably in these embodiments each R^{10a} may be independently selected from: fluoro, chloro, methyl, —CF₃, methoxy, —OCF₃ and —OCHF₂.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments the compound is of the formula (XXI), (XXIa), (XII), (XIIa), (XXXIII), (XXXIIIa), (XXXIIIb), (XXXIIIc) (XXXIV), (XXXIVa), (XXXIVb) or (XXXIVc) wherein the group of the formula:

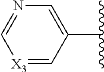

is selected from:

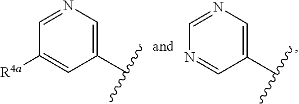

wherein R^{4a} is selected from: halo, —CN, C₁₋₆ alkyl, C₁₋₆ haloalkyl, —OR⁵, —NR⁵R⁶, —C(O)R⁵, —C(O)OR⁵, and —C(O)NR⁵R⁶; and each R¹⁰ (or R^{10a} as appropriate for each formula) is independently as defined in any of 190 to 201.

Suitably in these embodiments R^{4a} is selected from: halo, —CN and C₁₋₃ alkyl. For example, it may be that R^{4a} is selected from: F, —CN and methyl.

Thus it may be that the group of the formula:

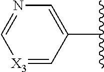

is selected from:

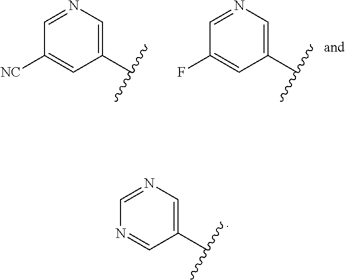 [SMILES: *c1cncc(C#N)c1 ; *c1cncc(F)c1 ; *c1cncnc1]

Thus it may be that the group of the formula:

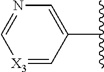

is selected from:

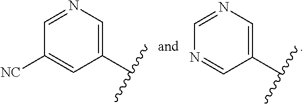 [SMILES: *c1cncc(C#N)c1 ; *c1cncnc1]

Suitably in these embodiments the group of the formula:

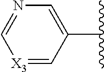

is selected from:

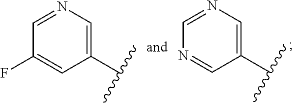 [SMILES: *c1cncc(F)c1 ; *c1cncnc1]

and
each R¹⁰ (or R^{10a} as appropriate for each formula) is independently selected from: halo, C₁₋₃ alkyl, C₁₋₃ haloalkyl, —OC₁₋₃ alkyl and —OC₁₋₃ haloalkyl. Thus each R^{10a} may be independently selected from: fluoro, chloro, methyl, —CF₃, methoxy and —OCF₃.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments the compound is of the formula (I), (Ia), (II), (IIa), (XXV), (XXVa), (XXVI), (XXVIa), (XXVII), (XXVIIa), (XXVIII), (XXVIIIa), (XXIX), (XXX) and (XXXa), Ring A is selected from:

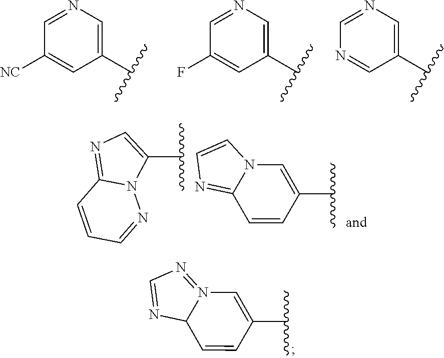

and L is a bond.

Suitably in these embodiments L is a bond; R² (when present) is H; and R³ is selected from methyl, ethyl and —CH₂CH₂F. Suitably in these embodiments L is a bond; R² (when present) is H; and R³ is methyl. Suitably in these embodiments L is a bond; R² (when present) is H; and R³ is ethyl. Suitably in these embodiments L is a bond; R² (when present) is H; and R³ is —CH₂CH₂F.

In certain embodiments the compound is of the formula (XVII), (XVIIa), (XVIII) or (XVIIIa), Ring D is selected from: benzene, pyridine, pyrimidine, pyridazine and pyrazine, and Ring E is selected from: pyrrole, imidazole, pyrazole, triazole and tetrazole, and wherein the 9-membered fused bicyclic heteroaryl ring formed by Ring D and Ring E is attached to -L- by a ring atom in Ring D.

In certain embodiments the compound is of the formula (XVII), (XVIIa), (XVIII) or (XVIIIa), the group of the formula:

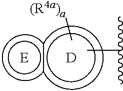

is selected from:

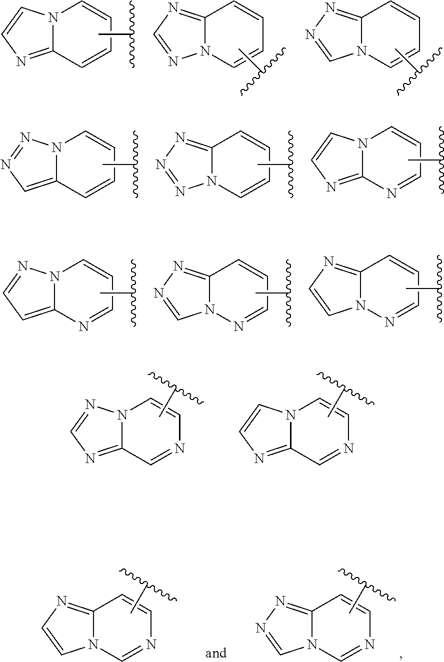

each of which is optionally substituted with 1, 2 or 3 (e.g. 1 or 2) R^{4a}, wherein each R^{4a} is independently as defined in any of 154 to 160, and wherein the 9-membered fused bicyclic heteroaryl ring formed by Ring D and Ring E is attached to -L- by a ring atom in Ring D.

In certain embodiments the compound is of the formula (XVII), (XVIIa), (XVIII) or (XVIIIa), the group of the formula:

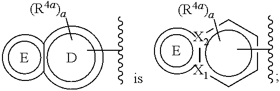

wherein: X₁ and X₂ are each independently N, or C, provided at least one of X₁ and X₂ is N; and Ring E is a fused 5-membered heteroaryl containing at least one (e.g. 1, 2, or 3) ring nitrogen, and wherein the 9-membered fused bicyclic heteroaryl ring formed by Ring D and Ring E is attached to -L- by a ring atom in Ring D.

Thus it may be that

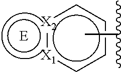

is selected from:

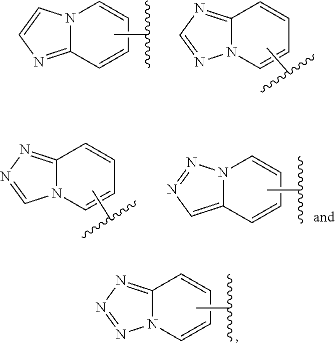

each of which is optionally substituted with 1, 2 or 3 (e.g. 1 or 2) R^{4a}, wherein each R^{4a} is independently as defined in any of 154 to 160. For example, the 9-membered fused bicyclic heteroaryl ring formed by Ring D and Ring E is selected from:

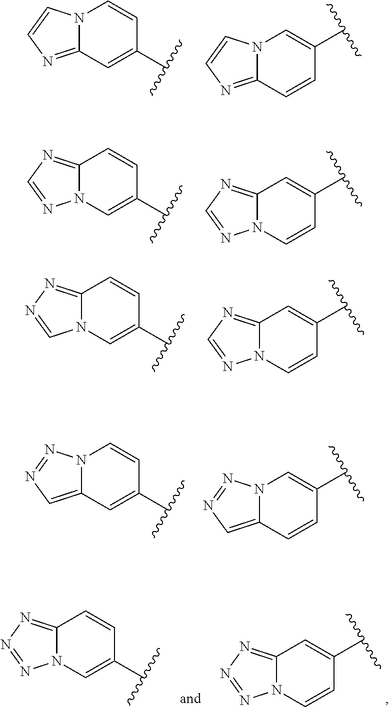

each of which is optionally substituted with 1, 2 or 3 (e.g. 1 or 2) R^{4a}, wherein each R^{4a} is independently as defined in any of 154 to 160.

Suitably in these embodiments of the formulae (XVII), (XVIIa), (XVIII) and (XVIIIa), each R^{4a} is independently selected from: halo, C₁₋₃ alkyl and —OC₁₋₃ alkyl. For example, each R^{4a} is independently selected from: F, Cl, methyl and methoxy.

Suitably in these embodiments of the formulae (XVII), (XVIIa), (XVIII) and (XVIIIa) the fused bicyclic heteroaryl formed by Ring D and Ring E is unsubstituted.

Suitably in these embodiments of these embodiments of the formulae (XVII), (XVIIa), (XVIII) and (XVIIIa), L is a bond; R² (when present) is H; and R³ is selected from methyl, ethyl and —CH₂CH₂F. Thus it may be that L is a bond; R² (when present) is H; and R³ is methyl. It may be that L is a bond; R² (when present) is H; and R³ is ethyl. It may be that L is a bond; R² (when present) is H; and R³ is —CH₂CH₂F.

In certain embodiments the compound is of the formula (XIX), (XIXa), (XX) or (XXa) Ring F is selected from: pyrrole, imidazole, pyrazole, triazole and tetrazole and Ring G is selected from: benzene, pyridine, pyrimidine, pyridazine and pyrazine, and wherein the 9-membered fused bicyclic heteroaryl ring formed by Ring F and Ring G is attached to -L- by a ring atom in Ring F.

In certain embodiments the compound is of the formula (XIX), (XIXa), (XX) or (XXa), the group of the formula:

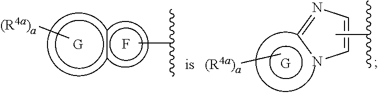

wherein the 9-membered fused bicyclic heteroaryl ring formed by Ring G and the imidazole ring is attached to -L- by a ring atom in the imidazole ring.

Thus it may be that

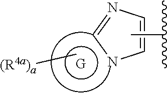

is selected from:

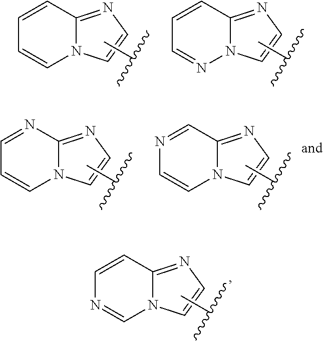

each of which is optionally substituted with 1, 2 or 3 (e.g. 1 or 2) R^{4a}, wherein each R^{4a} is independently as defined in any of 154 to 160, wherein the 9-membered fused bicyclic heteroaryl ring formed by Ring G and the imidazole ring is attached to -L- by a ring atom in the imidazole ring. For example, the 9-membered fused bicyclic heteroaryl ring formed by Ring G and the imidazole ring is selected from:

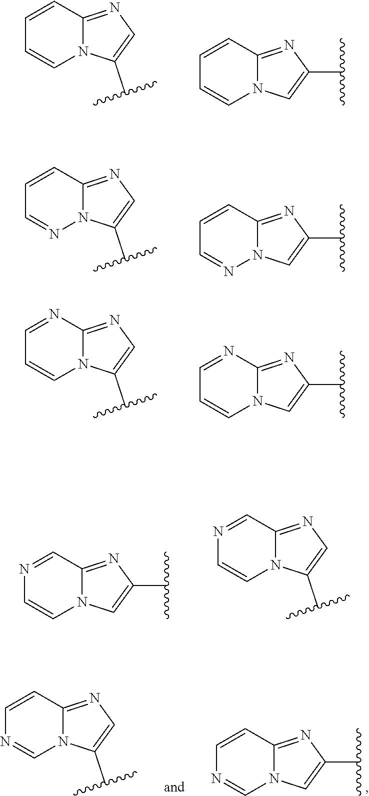

each of which is optionally substituted with 1, 2 or 3 (e.g. 1 or 2) R^{4a}, wherein each R^{4a} is independently as defined in any of 154 to 160.

Suitably in these embodiments of formulae (XIX), (XIXa), (XX) or (XXa) each R^{4a} is independently selected from: halo, C₁₋₃ alkyl and —OC₁₋₃ alkyl. For example, each R^{4a} is independently selected from: F, Cl, methyl and methoxy.

Suitably in these embodiments of the formulae (XIX), (XIXa), (XX) or (XXa) the fused bicyclic heteroaryl formed by Ring F and Ring G is unsubstituted.

Suitably in these embodiments of the formulae (XIX), (XIXa), (XX) or (XXa), L is a bond; R² (when present) is H; and R³ is selected from: methyl, ethyl and —CH₂CH₂F. Thus it may be that L is a bond; R² (when present) is H; and R³ is methyl. It may be that L is a bond; R² (when present) is H; and R³ is ethyl. It may be that L is a bond; R² (when present) is H; and R³ is —CH₂CH₂F.

In another embodiment there is provided a compound selected from Compound List 1, or a pharmaceutically acceptable salt thereof:

Compound List 1

| Example | Structure | IUPAC Name |
|---|---|---|
| 1 | 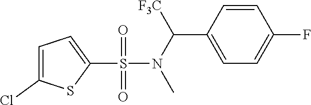 [SMILES: CN(C(c1ccc(F)cc1)C(F)(F)F)S(=O)(=O)c1ccc(Cl)s1] | 5-chloro-N-methyl-N-(2,2,2-trifluoro-1-(4- fluorophenyl)ethyl)thiophene-2-sulfonamide |
| 2 | 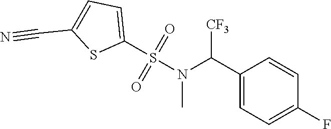 [SMILES: CN(C(c1ccc(F)cc1)C(F)(F)F)S(=O)(=O)c1ccc(C#N)s1] | 5-cyano-N-methyl-N-(2,2,2-trifluoro-1-(4- fluorophenyl)ethyl)thiophene-2-sulfonamide |
| 3 | 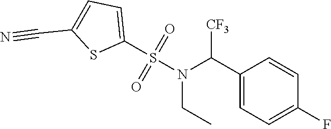 [SMILES: CCN(C(c1ccc(F)cc1)C(F)(F)F)S(=O)(=O)c1ccc(C#N)s1] | 5-cyano-N-ethyl-N-(2,2,2-trifluoro-1-(4- fluorophenyl)ethyl)thiophene-2-sulfonamide |
| 4 | 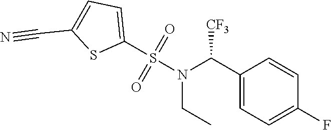 [SMILES: CCN([C@@H](c1ccc(F)cc1)C(F)(F)F)S(=O)(=O)c1ccc(C#N)s1] | (S)-5-cyano-N-ethyl-N-(2,2,2-trifluoro-1-(4- fluorophenyl)ethyl)thiophene-2-sulfonamide |
| 5 | 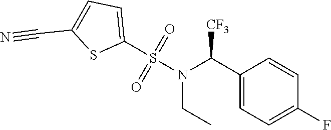 [SMILES: CCN([C@H](c1ccc(F)cc1)C(F)(F)F)S(=O)(=O)c1ccc(C#N)s1] | (R)-5-cyano-N-ethyl-N-(2,2,2-trifluoro-1-(4- fluorophenyl)ethyl)thiophene-2-sulfonamide |
| 6 | 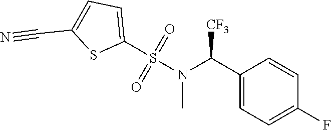 [SMILES: CN([C@H](c1ccc(F)cc1)C(F)(F)F)S(=O)(=O)c1ccc(C#N)s1] | (R)-5-cyano-N-methyl-N-(2,2,2-trifluoro-1- (4-fluorophenyl)ethyl)thiophene-2- sulfonamide |
| 7 | 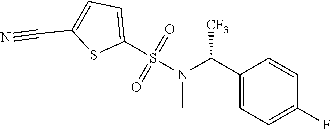 [SMILES: CN([C@@H](c1ccc(F)cc1)C(F)(F)F)S(=O)(=O)c1ccc(C#N)s1] | (S)-5-cyano-N-methyl-N-(2,2,2-trifluoro-1- (4-fluorophenyl)ethyl)thiophene-2- sulfonamide |
| 8 | 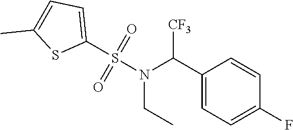 [SMILES: CCN(C(c1ccc(F)cc1)C(F)(F)F)S(=O)(=O)c1ccc(C)s1] | N-ethyl-5-methyl-N-(2,2,2-trifluoro-1-(4- fluorophenyl)ethyl)thiophene-2-sulfonamide |
| 9 | 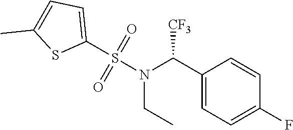 [SMILES: CCN([C@@H](c1ccc(F)cc1)C(F)(F)F)S(=O)(=O)c1ccc(C)s1] | (S)-N-ethyl-5-methyl-N-(2,2,2-trifluoro-1-(4- fluorophenyl)ethyl)thiophene-2-sulfonamide |
| 10 | 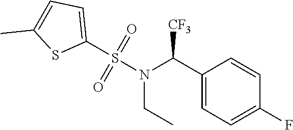 [SMILES: CCN([C@H](c1ccc(F)cc1)C(F)(F)F)S(=O)(=O)c1ccc(C)s1] | (R)-N-ethyl-5-methyl-N-(2,2,2-trifluoro-1-(4- fluorophenyl)ethyl)thiophene-2-sulfonamide |
| 11 | 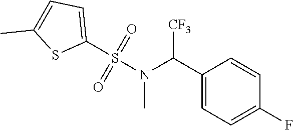 [SMILES: Cc1ccc(S(=O)(=O)N(C)C(c2ccc(F)cc2)C(F)(F)F)s1] | N,5-dimethyl-N-(2,2,2-trifluoro-1-(4- fluorophenyl)ethyl)thiophene-2-sulfonamide |
| 12 | 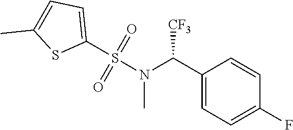 [SMILES: Cc1ccc(S(=O)(=O)N(C)[C@@H](c2ccc(F)cc2)C(F)(F)F)s1] | (S)-N,5-dimethyl-N-(2,2,2-trifluoro-1-(4- fluorophenyl)ethyl)thiophene-2-sulfonamide |
| 13 | 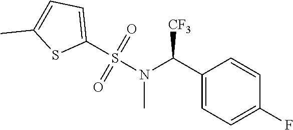 [SMILES: Cc1ccc(S(=O)(=O)N(C)[C@H](c2ccc(F)cc2)C(F)(F)F)s1] | (R)-N,5-dimethyl-N-(2,2,2-trifluoro-1-(4- fluorophenyl)ethyl)thiophene-2-sulfonamide |
| 14 | 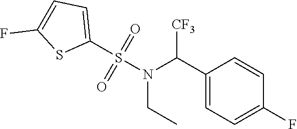 [SMILES: CCN(C(c1ccc(F)cc1)C(F)(F)F)S(=O)(=O)c1ccc(F)s1] | N-ethyl-5-fluoro-N-(2,2,2-trifluoro-1-(4- fluorophenyl)ethyl)thiophene-2-sulfonamide |
| 15 | 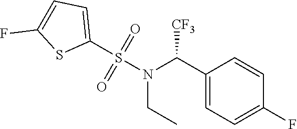 [SMILES: CCN([C@@H](c1ccc(F)cc1)C(F)(F)F)S(=O)(=O)c1ccc(F)s1] | (S)-N-ethyl-5-fluoro-N-(2,2,2-trifluoro-1-(4- fluorophenyl)ethyl)thiophene-2-sulfonamide |
| 16 | 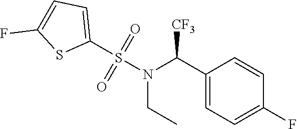 [SMILES: CCN([C@H](c1ccc(F)cc1)C(F)(F)F)S(=O)(=O)c1ccc(F)s1] | (R)-N-ethyl-5-fluoro-N-(2,2,2-trifluoro-1-(4- fluorophenyl)ethyl)thiophene-2-sulfonamide |
| 17 | 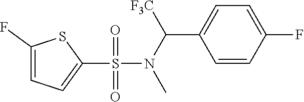 [SMILES: CN(C(c1ccc(F)cc1)C(F)(F)F)S(=O)(=O)c1ccc(F)s1] | 5-fluoro-N-methyl-N-(2,2,2-trifluoro-1-(4- fluorophenyl)ethyl)thiophene-2-sulfonamide |
| 18 | 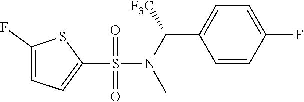 [SMILES: CN([C@@H](c1ccc(F)cc1)C(F)(F)F)S(=O)(=O)c1ccc(F)s1] | (S)-5-fluoro-N-methyl-N-(2,2,2-trifluoro-1-(4- fluorophenyl)ethyl)thiophene-2-sulfonamide |
| 19 | 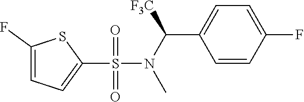 [SMILES: CN([C@H](c1ccc(F)cc1)C(F)(F)F)S(=O)(=O)c1ccc(F)s1] | (R)-5-fluoro-N-methyl-N-(2,2,2-trifluoro-1-(4- fluorophenyl)ethyl)thiophene-2-sulfonamide |
| 20 | 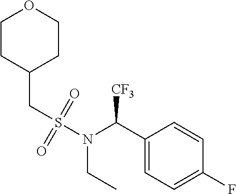 [SMILES: CCN([C@H](c1ccc(F)cc1)C(F)(F)F)S(=O)(=O)CC1CCOCC1] | (R)-N-ethyl-1-(tetrahydro-2H-pyran-4-yl)-N- (2,2,2-trifluoro-1-(4- fluorophenyl)ethyl)methanesulfonamide |
| 21 | 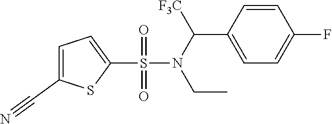 [SMILES: CCN(C(c1ccc(F)cc1)C(F)(F)F)S(=O)(=O)c1ccc(C#N)s1] | 5-cyano-N-ethyl-N-(2,2,2-trifluoro-1-(4- fluorophenyl)ethyl) thiophene-2-sulfonamide |
| 22 | 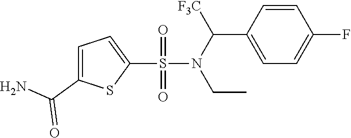 [SMILES: CCN(C(c1ccc(F)cc1)C(F)(F)F)S(=O)(=O)c1ccc(C(N)=O)s1] | 5-(N-ethyl-N-(2,2,2-trifluoro-1-(4- fluorophenyl)ethyl)sulfamoyl)thiophene-2- carboxamide |
| 23 | 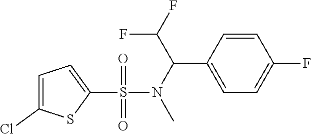 [SMILES: CN(C(c1ccc(F)cc1)C(F)F)S(=O)(=O)c1ccc(Cl)s1] | 5-chloro-N-(2,2-difluoro-1-(4- fluorophenyl)ethyl)-N-methylthiophene-2- sulfonamide |
| 24 | 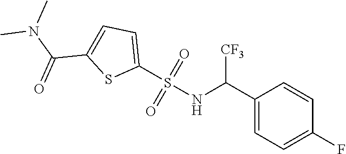 [SMILES: CN(C)C(=O)c1ccc(S(=O)(=O)NC(c2ccc(F)cc2)C(F)(F)F)s1] | N,N-dimethyl-5-(N-(2,2,2-trifluoro-1-(4- fluorophenyl)ethyl)sulfamoyl)thiophene-2- carboxamide |
| 25 | 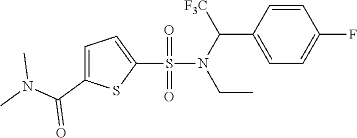 [SMILES: CCN(C(c1ccc(F)cc1)C(F)(F)F)S(=O)(=O)c1ccc(C(=O)N(C)C)s1] | 5-(N-ethyl-N-(2,2,2-trifluoro-1-(4- fluorophenyl)ethyl)sulfamoyl)-N,N- dimethylthiophene-2-carboxamide |
| 26 | 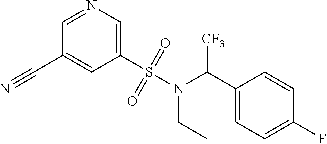 [SMILES: CCN(C(c1ccc(F)cc1)C(F)(F)F)S(=O)(=O)c1cncc(C#N)c1] | 5-cyano-N-ethyl-N-(2,2,2-trifluoro-1-(4- fluorophenyl)ethyl)pyridine-3-sulfonamide |
| 27 | 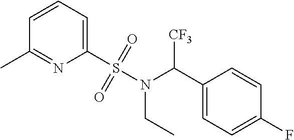 [SMILES: CCN(C(c1ccc(F)cc1)C(F)(F)F)S(=O)(=O)c1cccc(C)n1] | N-ethyl-6-methyl-N-(2,2,2-trifluoro-1-(4- fluorophenyl)ethyl)pyridine-2-sulfonamide |
| 28 | 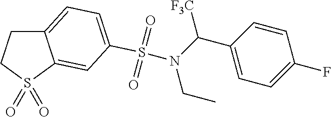 [SMILES: CCN(C(c1ccc(F)cc1)C(F)(F)F)S(=O)(=O)c1ccc2c(c1)S(=O)(=O)CC2] | N-ethyl-N-(2,2,2-trifluoro-1-(4- fluorophenyl)ethyl)-2,3- dihydrobenzo[b]thiophene-6-sulfonamide 1,1-dioxide |
| 29 | 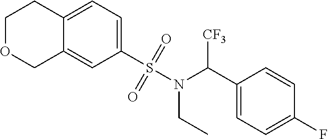 [SMILES: CCN(C(c1ccc(F)cc1)C(F)(F)F)S(=O)(=O)c1ccc2c(c1)COCC2] | N-ethyl-N-(2,2,2-trifluoro-1-(4- fluorophenyl)ethyl)isochromane-7- sulfonamide |
| 30 | 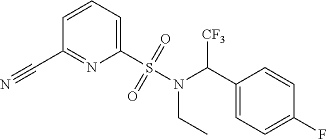 [SMILES: CCN(C(c1ccc(F)cc1)C(F)(F)F)S(=O)(=O)c1cccc(C#N)n1] | 6-cyano-N-ethyl-N-(2,2,2-trifluoro-1-(4- fluorophenyl)ethyl)pyridine-2-sulfonamide |
| 31 | 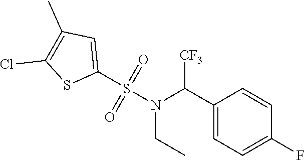 [SMILES: CCN(C(c1ccc(F)cc1)C(F)(F)F)S(=O)(=O)c1cc(C)c(Cl)s1] | 5-chloro-N-ethyl-4-methyl-N-(2,2,2-trifluoro- 1-(4-fluorophenyl)ethyl)thiophene-2- sulfonamide |
| 32 | 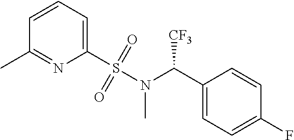 | (S)-N-ethyl-6-methyl-N-(2,2,2-trifluoro-1-(4- fluorophenyl)ethyl)pyridine-2-sulfonamide |
| 33 | 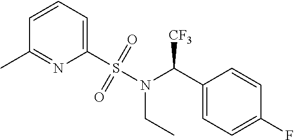 | (R)-N-ethyl-6-methyl-N-(2,2,2-trifluoro-1-(4- fluorophenyl)ethyl)pyridine-2-sulfonamide |
| 34 | 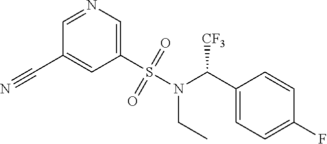 | (S)-5-cyano-N-ethyl-N-(2,2,2-trifluoro-1-(4- fluorophenyl)ethyl)pyridine-3-sulfonamide |
| 35 | 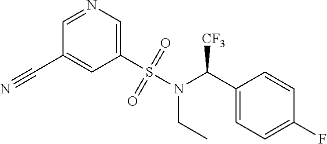 | (R)-5-cyano-N-ethyl-N-(2,2,2-trifluoro-1-(4- fluorophenyl)ethyl)pyridine-3-sulfonamide |
| 36 | 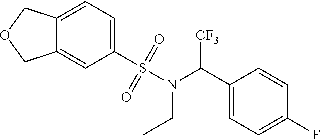 [SMILES: CCN(C(c1ccc(F)cc1)C(F)(F)F)S(=O)(=O)c1ccc2c(c1)COC2] | N-ethyl-N-(2,2,2-trifluoro-1-(4- fluorophenyl)ethyl)-1,3- dihydroisobenzofuran-5-sulfonamide |
| 37 | 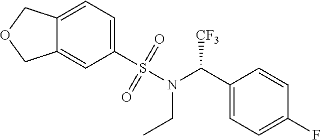 | (S)-N-ethyl-N-(2,2,2-trifluoro-1-(4- fluorophenyl)ethyl)-1,3- dihydroisobenzofuran-5-sulfonamide |
| 38 | 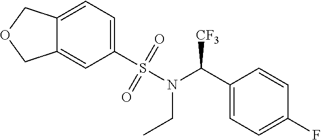 | (R)-N-ethyl-N-(2,2,2-trifluoro-1-(4- fluorophenyl)ethyl)-1,3- dihydroisobenzofuran-5-sulfonamide |
| 39 | 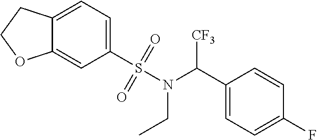 [SMILES: CCN(C(c1ccc(F)cc1)C(F)(F)F)S(=O)(=O)c1ccc2c(c1)OCC2] | N-ethyl-N-(2,2,2-trifluoro-1-(4- fluorophenyl)ethyl)-2,3-dihydrobenzofuran- 6-sulfonamide |
| 40 | 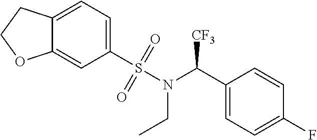 | (R)-N-ethyl-N-(2,2,2-trifluoro-1-(4- fluorophenyl)ethyl)-2,3-dihydrobenzofuran- 6-sulfonamide |
| 41 | 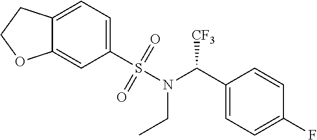 | (S)-N-ethyl-N-(2,2,2-trifluoro-1-(4- fluorophenyl)ethyl)-2,3-dihydrobenzofuran- 6-sulfonamide |
| 42 | 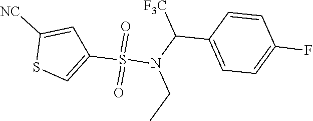 [SMILES: CCN(C(c1ccc(F)cc1)C(F)(F)F)S(=O)(=O)c1csc(C#N)c1] | 5-cyano-N-ethyl-N-(2,2,2-trifluoro-1-(4- fluorophenyl)ethyl)thiophene-3-sulfonamide |
| 43 | 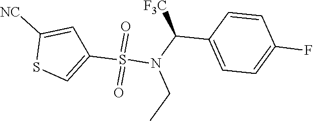 [SMILES: CCN([C@H](c1ccc(F)cc1)C(F)(F)F)S(=O)(=O)c1csc(C#N)c1] | (R)-5-cyano-N-ethyl-N-(2,2,2-trifluoro-1-(4- fluorophenyl)ethyl)thiophene-3-sulfonamide |
| 44 | 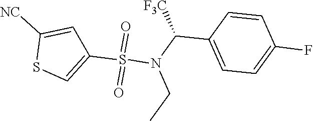 [SMILES: CCN([C@@H](c1ccc(F)cc1)C(F)(F)F)S(=O)(=O)c1csc(C#N)c1] | (S)-5-cyano-N-ethyl-N-(2,2,2-trifluoro-1-(4- fluorophenyl)ethyl)thiophene-3-sulfonamide |
| 45 | 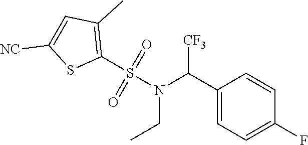 [SMILES: CCN(C(c1ccc(F)cc1)C(F)(F)F)S(=O)(=O)c1sc(C#N)cc1C] | 5-cyano-N-ethyl-3-methyl-N-(2,2,2-trifluoro- 1-(4-fluorophenyl)ethyl)thiophene-2- sulfonamide |
| 46 | 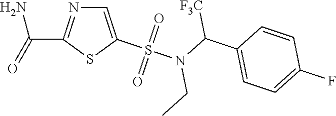 [SMILES: CCN(C(c1ccc(F)cc1)C(F)(F)F)S(=O)(=O)c1cnc(C(N)=O)s1] | 5-(N-ethyl-N-(2,2,2-trifluoro-1-(4- fluorophenyl)ethyl)sulfamoyl)thiazole-2- carboxamide |
| 47 | 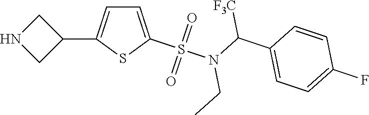 [SMILES: CCN(C(c1ccc(F)cc1)C(F)(F)F)S(=O)(=O)c1ccc(C2CNC2)s1] | 5-(azetidin-3-yl)-N-ethyl-N-(2,2,2-trifluoro-1- (4-fluorophenyl)ethyl)thiophene-2- sulfonamide |
| 48 | 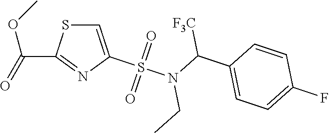 [SMILES: CCN(C(c1ccc(F)cc1)C(F)(F)F)S(=O)(=O)c1csc(C(=O)OC)n1] | methyl 4-(N-ethyl-N-(2,2,2-trifluoro-1-(4- fluorophenyl)ethyl)sulfamoyl)thiazole-2- carboxylate |
| 49 | 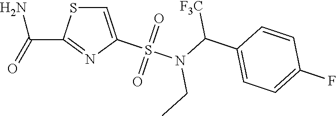 [SMILES: CCN(C(c1ccc(F)cc1)C(F)(F)F)S(=O)(=O)c1csc(C(N)=O)n1] | 4-(N-ethyl-N-(2,2,2-trifluoro-1-(4- fluorophenyl)ethyl)sulfamoyl)thiazole-2- carboxamide |
| 50 | 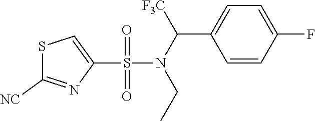 [SMILES: CCN(C(c1ccc(F)cc1)C(F)(F)F)S(=O)(=O)c1csc(C#N)n1] | 2-cyano-N-ethyl-N-(2,2,2-trifluoro-1-(4- fluorophenyl)ethyl)thiazole-4-sulfonamide |
| 51 | 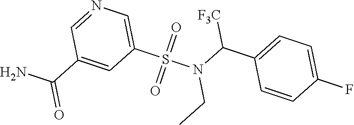 [SMILES: CCN(C(c1ccc(F)cc1)C(F)(F)F)S(=O)(=O)c1cncc(C(N)=O)c1] | 5-(N-ethyl-N-(2,2,2-trifluoro-1-(4- fluorophenyl)ethyl)sulfamoyl)nicotinamide |
| 52 | 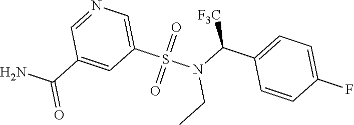 [SMILES: CCN([C@H](c1ccc(F)cc1)C(F)(F)F)S(=O)(=O)c1cncc(C(N)=O)c1] | (R)-5-(N-ethyl-N-(2,2,2-trifluoro-1-(4- fluorophenyl)ethyl)sulfamoyl)nicotinamide |
| 53 | 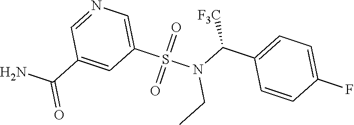 [SMILES: CCN([C@@H](c1ccc(F)cc1)C(F)(F)F)S(=O)(=O)c1cncc(C(N)=O)c1] | (S)-5-(N-ethyl-N-(2,2,2-trifluoro-1-(4- fluorophenyl)ethyl)sulfamoyl)nicotinamide |
| 54 | 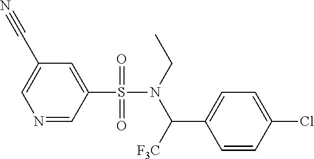 [SMILES: CCN(C(c1ccc(Cl)cc1)C(F)(F)F)S(=O)(=O)c1cncc(C#N)c1] | N-(1-(4-chlorophenyl)-2,2,2-trifluoroethyl)-5- cyano-N-ethylpyridine-3-sulfonamide |
| 55 | 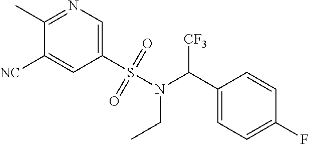 [SMILES: CCN(C(c1ccc(F)cc1)C(F)(F)F)S(=O)(=O)c1cnc(C)c(C#N)c1] | 5-cyano-N-ethyl-6-methyl-N-[2,2,2-trifluoro- 1-(4-fluorophenyl)ethyl]pyridine-3- sulfonamide |
| 56 | 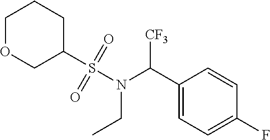 [SMILES: CCN(C(c1ccc(F)cc1)C(F)(F)F)S(=O)(=O)C1CCCOC1] | N-ethyl-N-(2,2,2-trifluoro-1-(4- fluorophenyl)ethyl)tetrahydro-2H-pyran-3- sulfonamide |
| 57 | 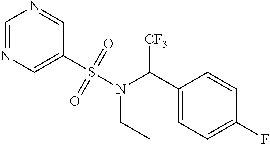 [SMILES: CCN(C(c1ccc(F)cc1)C(F)(F)F)S(=O)(=O)c1cncnc1] | N-ethyl-N-(2,2,2-trifluoro-1-(4- fluorophenyl)ethyl)pyrimidine-5-sulfonamide |
| 58 | 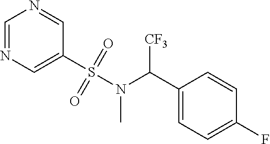 [SMILES: CN(C(c1ccc(F)cc1)C(F)(F)F)S(=O)(=O)c1cncnc1] | N-methyl-N-(2,2,2-trifluoro-1-(4- fluorophenyl)ethyl)pyrimidine-5-sulfonamide |
| 59 | 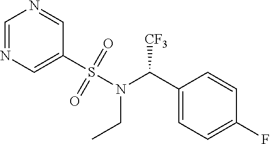 | (S)-N-ethyl-N-(2,2,2-trifluoro-1-(4- fluorophenyl)ethyl)pyrimidine-5-sulfonamide |
| 60 | 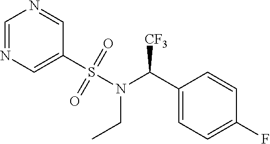 | (R)-N-ethyl-N-(2,2,2-trifluoro-1-(4- fluorophenyl)ethyl)pyrimidine-5-sulfonamide |
| 61 | 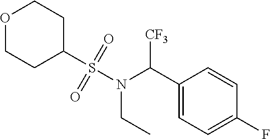 [SMILES: CCN(C(c1ccc(F)cc1)C(F)(F)F)S(=O)(=O)C1CCOCC1] | N-ethyl-N-(2,2,2-trifluoro-1-(4- fluorophenyl)ethyl)tetrahydro-2H-pyran-4- sulfonamide |
| 62 | 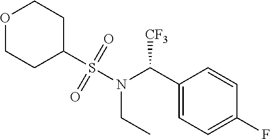 | (S)-N-ethyl-N-(2,2,2-trifluoro-1-(4- fluorophenyl)ethyl)tetrahydro-2H-pyran-4- sulfonamide |
| 63 | 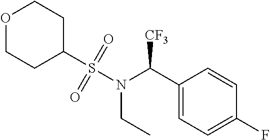 | (R)-N-ethyl-N-(2,2,2-trifluoro-1-(4- fluorophenyl)ethyl)tetrahydro-2H-pyran-4- sulfonamide |
| 64 | 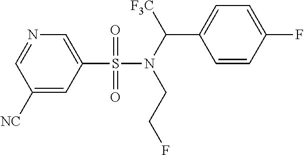 [SMILES: N#Cc1cncc(S(=O)(=O)N(CCF)C(c2ccc(F)cc2)C(F)(F)F)c1] | 5-Cyano-N-(2-fluoroethyl)-N-(2,2,2-trifluoro- 1-(4-fluorophenyl)ethyl)pyridine-3- sulfonamide |
| 65 | 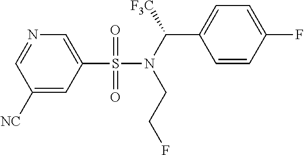 [SMILES: N#Cc1cncc(S(=O)(=O)N(CCF)[C@@H](c2ccc(F)cc2)C(F)(F)F)c1] | (S)-5-Cyano-N-(2-fluoroethyl)-N-(2,2,2- trifluoro-1-(4-fluorophenyl)ethyl)pyridine-3- sulfonamide |
| 66 | 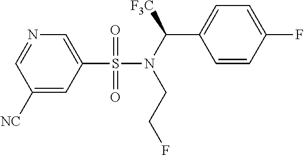 [SMILES: N#Cc1cncc(S(=O)(=O)N(CCF)[C@H](c2ccc(F)cc2)C(F)(F)F)c1] | (R)-5-Cyano-N-(2-fluoroethyl)-N-(2,2,2- trifluoro-1-(4-fluorophenyl)ethyl)pyridine-3- sulfonamide |
| 67 | 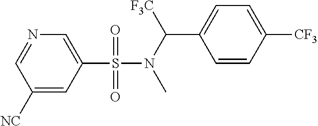 [SMILES: CN(C(c1ccc(C(F)(F)F)cc1)C(F)(F)F)S(=O)(=O)c1cncc(C#N)c1] | 5-cyano-N-methyl-N-(2,2,2-trifluoro-1-(4- (trifluoromethyl)phenyl)ethyl)pyridine-3- sulfonamide |
| 68 | 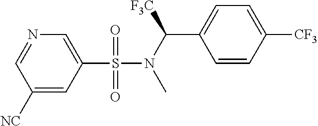 [SMILES: CN([C@H](c1ccc(C(F)(F)F)cc1)C(F)(F)F)S(=O)(=O)c1cncc(C#N)c1] | (R)-5-cyano-N-methyl-N-(2,2,2-trifluoro-1- (4-(trifluoromethyl)phenyl)ethyl)pyridine-3- sulfonamide |
| 69 | 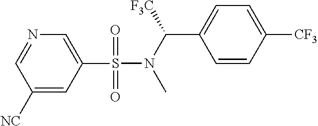 [SMILES: CN([C@@H](c1ccc(C(F)(F)F)cc1)C(F)(F)F)S(=O)(=O)c1cncc(C#N)c1] | (R)-5-cyano-N-methyl-N-(2,2,2-trifluoro-1- (4-(trifluoromethyl)phenyl)ethyl)pyridine-3- sulfonamide |
| 70 | 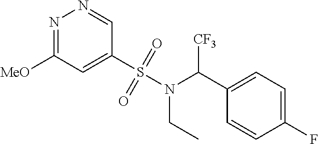 [SMILES: CCN(C(c1ccc(F)cc1)C(F)(F)F)S(=O)(=O)c1cnnc(OC)c1] | N-ethyl-6-methoxy-N-(2,2,2-trifluoro-1-(4- fluorophenyl)ethyl)pyridazine-4-sulfonamide |
| 71 | 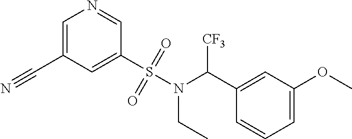 [SMILES: CCN(C(c1cccc(OC)c1)C(F)(F)F)S(=O)(=O)c1cncc(C#N)c1] | 5-Cyano-N-ethyl-N-(2,2,2-trifluoro-1-(3- methoxyphenyl)ethyl)pyridine-3- sulfonamide |
| 72 | 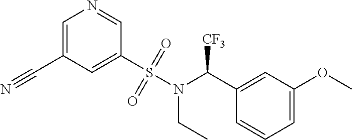 | (R)-5-Cyano-N-ethyl-N-(2,2,2-trifluoro-1-(3- methoxyphenyl)ethyl)pyridine-3- sulfonamide |
| 73 | 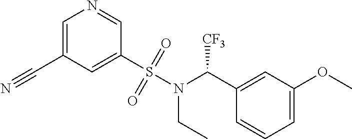 | (S)-5-Cyano-N-ethyl-N-(2,2,2-trifluoro-1-(3- methoxyphenyl)ethyl)pyridine-3- sulfonamide |
| 74 | 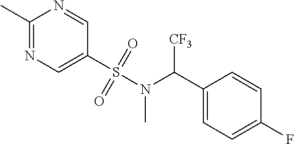 [SMILES: Cc1ncc(S(=O)(=O)N(C)C(c2ccc(F)cc2)C(F)(F)F)cn1] | N,2-dimethyl-N-(2,2,2-trifluoro-1-(4- fluorophenyl)ethyl)pyrimidine-5-sulfonamide |
| 75 | 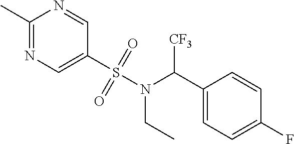 [SMILES: CCN(C(c1ccc(F)cc1)C(F)(F)F)S(=O)(=O)c1cnc(C)nc1] | N-ethyl-2-methyl-N-(2,2,2-trifluoro-1-(4- fluorophenyl)ethyl)pyrimidine-5-sulfonamide |
| 76 | 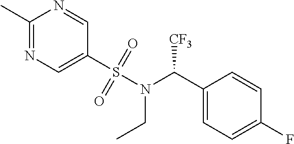 | (S)-N-ethyl-2-methyl-N-(2,2,2-trifluoro-1-(4- fluorophenyl)ethyl)pyrimidine-5-sulfonamide |
| 77 | 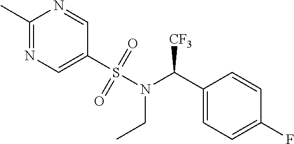 | (R)-N-ethyl-2-methyl-N-(2,2,2-trifluoro-1-(4- fluorophenyl)ethyl)pyrimidine-5-sulfonamide |
| 78 | 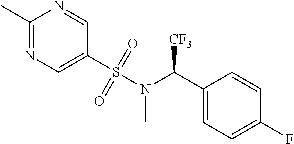 | (R)-N,2-dimethyl-N-(2,2,2-trifluoro-1-(4- fluorophenyl)ethyl)pyrimidine-5-sulfonamide |
| 79 | 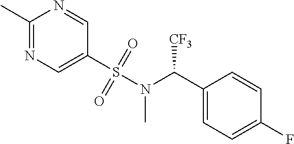 | (S)-N,2-dimethyl-N-(2,2,2-trifluoro-1-(4- fluorophenyl)ethyl)pyrimidine-5-sulfonamide |
| 80 | 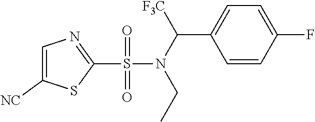 [SMILES: CCN(C(c1ccc(F)cc1)C(F)(F)F)S(=O)(=O)c1ncc(C#N)s1] | 5-cyano-N-ethyl-N-(2,2,2-trifluoro-1-(4- fluorophenyl)ethyl)thiazole-2-sulfonamide |
| 81 | 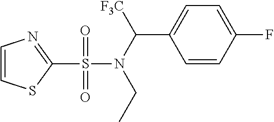 [SMILES: CCN(C(c1ccc(F)cc1)C(F)(F)F)S(=O)(=O)c1nccs1] | N-ethyl-N-(2,2,2-trifluoro-1-(4- fluorophenyl)ethyl)thiazole-2-sulfonamide |
| 82 | 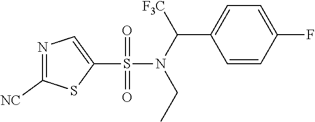 [SMILES: CCN(C(c1ccc(F)cc1)C(F)(F)F)S(=O)(=O)c1cnc(C#N)s1] | 2-Cyano-N-ethyl-N-(2,2,2-trifluoro-1-(4- fluorophenyl)ethyl)thiazole-5-sulfonamide |
| 83 | 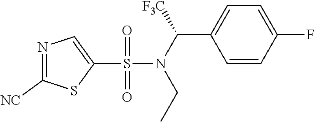 [SMILES: CCN([C@@H](c1ccc(F)cc1)C(F)(F)F)S(=O)(=O)c1cnc(C#N)s1] | (S)-2-Cyano-N-ethyl-N-(2,2,2-trifluoro-1-(4- fluorophenyl)ethyl)thiazole-5-sulfonamide |
| 84 | 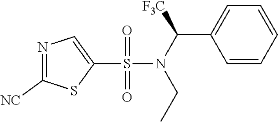 [SMILES: CCN([C@H](c1ccccc1)C(F)(F)F)S(=O)(=O)c1cnc(C#N)s1] | (R)-2-Cyano-N-ethyl-N-(2,2,2-trifluoro-1-(4- fluorophenyl)ethyl)thiazole-5-sulfonamide |
| 85 | 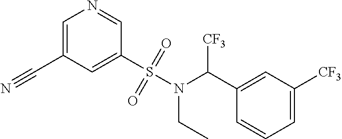 [SMILES: CCN(C(c1cccc(C(F)(F)F)c1)C(F)(F)F)S(=O)(=O)c1cncc(C#N)c1] | 5-cyano-N-ethyl-N-(2,2,2-trifluoro-1-(3- (trifluoromethyl)phenyl)ethyl)pyridine-3- sulfonamide |
| 86 | 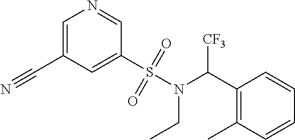 [SMILES: CCN(C(c1ccccc1C)C(F)(F)F)S(=O)(=O)c1cncc(C#N)c1] | 5-cyano-N-ethyl-N-(2,2,2-trifluoro-1-(o- tolyl)ethyl)pyridine-3-sulfonamide |
| 87 | 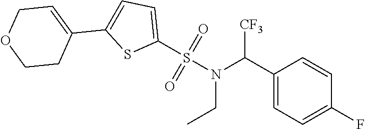 [SMILES: CCN(C(c1ccc(F)cc1)C(F)(F)F)S(=O)(=O)c1ccc(C2=CCOCC2)s1] | 5-(3,6-Dihydro-2H-pyran-4-yl)-N-ethyl-N- (2,2,2-trifluoro-1-(4- fluorophenyl)ethyl)thiophene-2-sulfonamide |
| 88 | 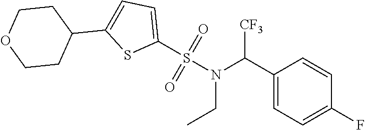 [SMILES: CCN(C(c1ccc(F)cc1)C(F)(F)F)S(=O)(=O)c1ccc(C2CCOCC2)s1] | N-ethyl-5-(tetrahydro-2H-pyran-4-yl)-N- (2,2,2-trifluoro-1-(4- fluorophenyl)ethyl)thiophene-2-sulfonamide |
| 89 | 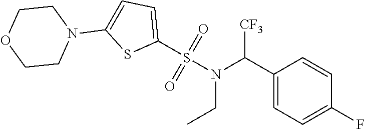 [SMILES: CCN(C(c1ccc(F)cc1)C(F)(F)F)S(=O)(=O)c1ccc(N2CCOCC2)s1] | N-ethyl-5-morpholino-N-(2,2,2-trifluoro-1-(4- fluorophenyl)ethyl)thiophene-2-sulfonamide |
| 90 | 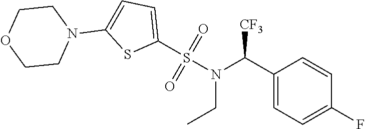 [SMILES: CCN([C@H](c1ccc(F)cc1)C(F)(F)F)S(=O)(=O)c1ccc(N2CCOCC2)s1] | (R)-N-ethyl-5-morpholino-N-(2,2,2-trifluoro- 1-(4-fluorophenyl)ethyl)thiophene-2- sulfonamide |
| 91 | 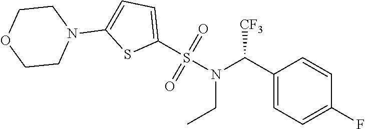 [SMILES: CCN([C@@H](c1ccc(F)cc1)C(F)(F)F)S(=O)(=O)c1ccc(N2CCOCC2)s1] | (S)-N-ethyl-5-morpholino-N-(2,2,2-trifluoro- 1-(4-fluorophenyl)ethyl)thiophene-2- sulfonamide |
| 92 | 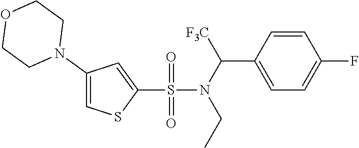 [SMILES: CCN(C(c1ccc(F)cc1)C(F)(F)F)S(=O)(=O)c1cc(N2CCOCC2)cs1] | N-ethyl-4-morpholino-N-(2,2,2-trifluoro-1-(4- fluorophenyl)ethyl)thiophene-2-sulfonamide |
| 93 | 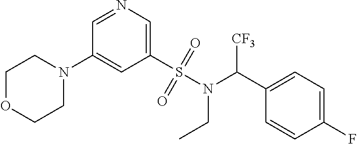 [SMILES: CCN(C(c1ccc(F)cc1)C(F)(F)F)S(=O)(=O)c1cncc(N2CCOCC2)c1] | N-ethyl-5-morpholino-N-(2,2,2-trifluoro-1-(4- fluorophenyl)ethyl)pyridine-3-sulfonamide |
| 94 | 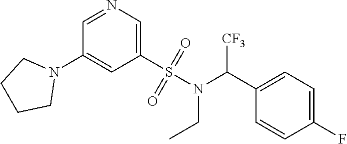 [SMILES: CCN(C(c1ccc(F)cc1)C(F)(F)F)S(=O)(=O)c1cncc(N2CCCC2)c1] | N-ethyl-5-(pyrrolidin-1-yl)-N-(2,2,2-trifluoro- 1-(4-fluorophenyl)ethyl)pyridine-3- sulfonamide |
| 95 | 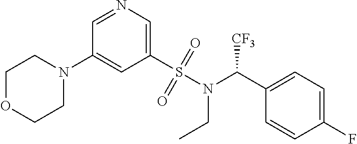 | (S)-N-ethyl-5-morpholino-N-(2,2,2-trifluoro- 1-(4-fluorophenyl)ethyl)pyridine-3- sulfonamide |
| 96 | 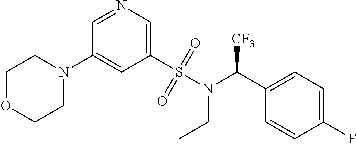 | (R)-N-ethyl-5-morpholino-N-(2,2,2-trifluoro- 1-(4-fluorophenyl)ethyl)pyridine-3- sulfonamide |
| 97 | 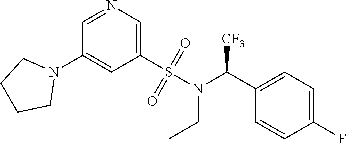 | (S)-N-ethyl-5-(pyrrolidin-1-yl)-N-(2,2,2- trifluoro-1-(4-fluorophenyl)ethyl)pyridine-3- sulfonamide |
| 98 | 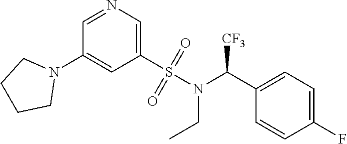 | (R)-N-ethyl-5-(pyrrolidin-1-yl)-N-(2,2,2- trifluoro-1-(4-fluorophenyl)ethyl)pyridine-3- sulfonamide |
| 99 | 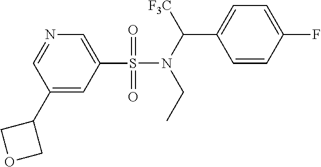 [SMILES: CCN(C(c1ccc(F)cc1)C(F)(F)F)S(=O)(=O)c1cncc(C2COC2)c1] | N-ethyl-5-(oxetan-3-yl)-N-(2,2,2-trifluoro-1- (4-fluorophenyl)ethyl)pyridine-3-sulfonamide |
| 100 | 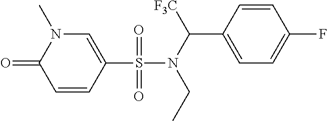 [SMILES: CCN(C(c1ccc(F)cc1)C(F)(F)F)S(=O)(=O)c1ccc(=O)n(C)c1] | N-ethyl-1-methyl-6-oxo-N-(2,2,2-trifluoro-1- (4-fluorophenyl)ethyl)-1,6-dihydropyridine-3- sulfonamide |
| 101 | 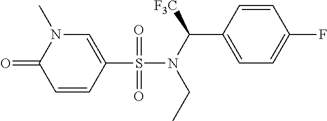 [SMILES: CCN([C@H](c1ccc(F)cc1)C(F)(F)F)S(=O)(=O)c1ccc(=O)n(C)c1] | (R)-N-ethyl-1-methyl-6-oxo-N-(2,2,2- trifluoro-1-(4-fluorophenyl)ethyl)-1,6- dihydropyridine-3-sulfonamide |
| 102 | 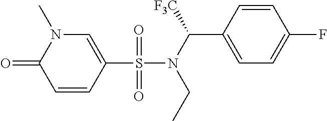 [SMILES: CCN([C@@H](c1ccc(F)cc1)C(F)(F)F)S(=O)(=O)c1ccc(=O)n(C)c1] | (S)-N-ethyl-1-methyl-6-oxo-N-(2,2,2- trifluoro-1-(4-fluorophenyl)ethyl)-1,6- dihydropyridine-3-sulfonamide |
| 103 | 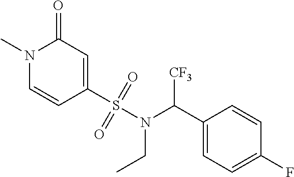 [SMILES: CCN(C(c1ccc(F)cc1)C(F)(F)F)S(=O)(=O)c1ccn(C)c(=O)c1] | N-ethyl-1-methyl-2-oxo-N-(2,2,2-trifluoro-1- (4-fluorophenyl)ethyl)-1,2-dihydropyridine-4- sulfonamide |
| 104 | 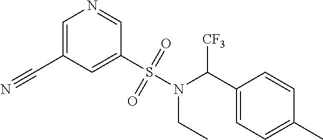 [SMILES: CCN(C(c1ccc(C)cc1)C(F)(F)F)S(=O)(=O)c1cncc(C#N)c1] | 5-Cyano-N-ethyl-N-(2,2,2-trifluoro-1-(p- tolyl)ethyl)pyridine-3-sulfonamide |
| 105 | 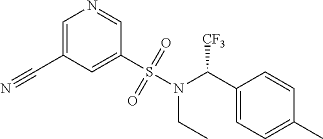 | (S)-5-Cyano-N-ethyl-N-(2,2,2-trifluoro-1-(p- tolyl)ethyl)pyridine-3-sulfonamide |
| 106 | 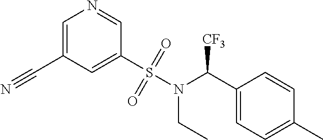 | (R)-5-Cyano-N-ethyl-N-(2,2,2-trifluoro-1-(p- tolyl)ethyl)pyridine-3-sulfonamide |
| 107 | 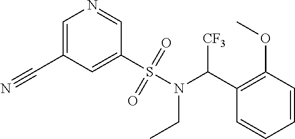 [SMILES: CCN(C(c1ccccc1OC)C(F)(F)F)S(=O)(=O)c1cncc(C#N)c1] | 5-Cyano-N-ethyl-N-(2,2,2-trifluoro-1-(2- methoxyphenyl)ethyl)pyridine-3- sulfonamide |
| 108 | 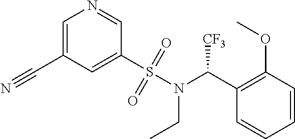 | (S)-5-cyano-N-ethyl-N-(2,2,2-trifluoro-1-(2- methoxyphenyl)ethyl)pyridine-3- sulfonamide |
| 109 | 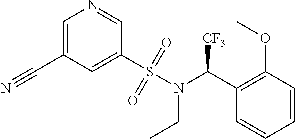 | (R)-5-cyano-N-ethyl-N-(2,2,2-trifluoro-1-(2- methoxyphenyl)ethyl)pyridine-3- sulfonamide |
| 110 | 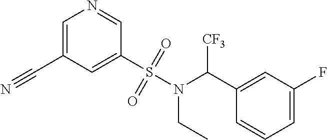 [SMILES: CCN(C(c1cccc(F)c1)C(F)(F)F)S(=O)(=O)c1cncc(C#N)c1] | 5-Cyano-N-ethyl-N-(2,2,2-trifluoro-1-(3- fluorophenyl)ethyl)pyridine-3-sulfonamide |
| 111 | 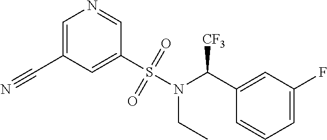 | (R)-5-Cyano-N-ethyl-N-(2,2,2-trifluoro-1-(3- fluorophenyl)ethyl)pyridine-3-sulfonamide |
| 112 | 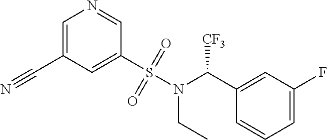 | (S)-5-Cyano-N-ethyl-N-(2,2,2-trifluoro-1-(3- fluorophenyl)ethyl)pyridine-3-sulfonamide |
| 113 | 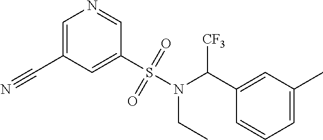 [SMILES: CCN(C(c1cccc(C)c1)C(F)(F)F)S(=O)(=O)c1cncc(C#N)c1] | 5-Cyano-N-ethyl-N-(2,2,2-trifluoro-1-(m- tolyl)ethyl)pyridine-3-sulfonamide |
| 114 | 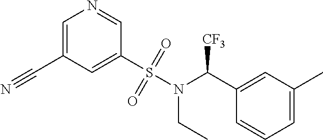 | (R)-5-Cyano-N-ethyl-N-(2,2,2-trifluoro-1-(m- tolyl)ethyl)pyridine-3-sulfonamide |
| 115 | 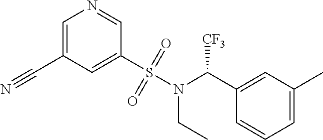 | (S)-5-Cyano-N-ethyl-N-(2,2,2-trifluoro-1-(m- tolyl)ethyl)pyridine-3-sulfonamide |
| 116 | 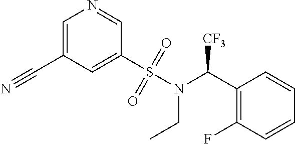 | (R)-5-Cyano-N-ethyl-N-(2,2,2-trifluoro-1-(2- fluorophenyl)ethyl)pyridine-3-sulfonamide |
| 117 | 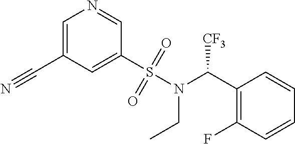 | (S)-5-Cyano-N-ethyl-N-(2,2,2-trifluoro-1-(2- fluorophenyl)ethyl)pyridine-3-sulfonamide |
| 118 | 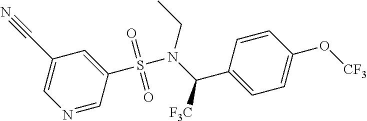 [SMILES: CCN([C@@H](c1ccc(OC(F)(F)F)cc1)C(F)(F)F)S(=O)(=O)c1cncc(C#N)c1] | (S)-5-Cyano-N-ethyl-N-(2,2,2-trifluoro-1-(4- (trifluoromethoxy)phenyl)ethyl)pyridine-3- sulfonamide |
| 119 | 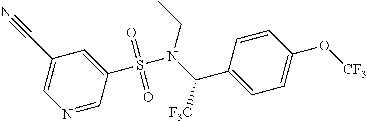 [SMILES: CCN([C@H](c1ccc(OC(F)(F)F)cc1)C(F)(F)F)S(=O)(=O)c1cncc(C#N)c1] | (R)-5-Cyano-N-ethyl-N-(2,2,2-trifluoro-1-(4- (trifluoromethoxy)phenyl)ethyl)pyridine-3- sulfonamide |
| 120 | 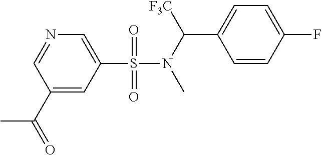 [SMILES: CC(=O)c1cncc(S(=O)(=O)N(C)C(c2ccc(F)cc2)C(F)(F)F)c1] | 5-Acetyl-N-ethyl-N-(2,2,2-trifluoro-1-(4- fluorophenyl)ethyl)pyridine-3-sulfonamide |
| 121 | 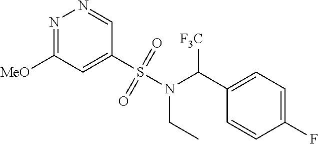 [SMILES: CCN(C(c1ccc(F)cc1)C(F)(F)F)S(=O)(=O)c1cnnc(OC)c1] | N-Ethyl-6-methoxy-N-(2,2,2-trifluoro-1-(4- fluorophenyl)ethyl)pyridazine-4-sulfonamide |
| 122 | 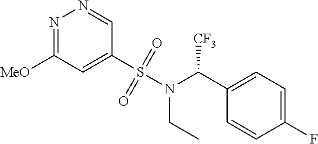 | (S)-N-Ethyl-6-methoxy-N-(2,2,2-trifluoro-1- (4-fluorophenyl)ethyl)pyridazine-4- sulfonamide |
| 123 | 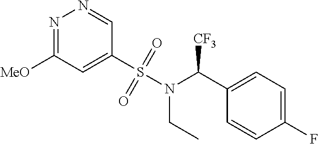 | (R)-N-Ethyl-6-methoxy-N-(2,2,2-trifluoro-1- (4-fluorophenyl)ethyl)pyridazine-4- sulfonamide |
| 124 | 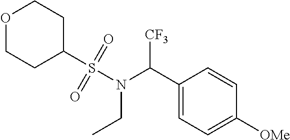 [SMILES: CCN(C(c1ccc(OC)cc1)C(F)(F)F)S(=O)(=O)C1CCOCC1] | N-Ethyl-N-(2,2,2-trifluoro-1-(4- methoxyphenyl)ethyl)tetrahydro-2H-pyran- 4-sulfonamide |
| 125 | 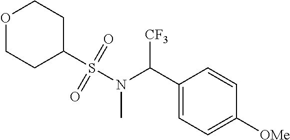 [SMILES: COc1ccc(C(N(C)S(=O)(=O)C2CCOCC2)C(F)(F)F)cc1] | N-Methyl-N-(2,2,2-trifluoro-1-(4- methoxyphenyl)ethyl)tetrahydro-2H-pyran- 4-sulfonamide |
| 126 | 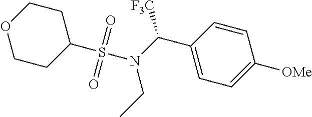 [SMILES: CCN([C@@H](c1ccc(OC)cc1)C(F)(F)F)S(=O)(=O)C1CCOCC1] | (S)-N-Ethyl-N-(2,2,2-trifluoro-1-(4- methoxyphenyl)ethyl)tetrahydro-2H-pyran- 4-sulfonamide |
| 127 | 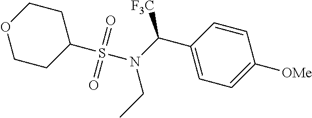 [SMILES: CCN([C@H](c1ccc(OC)cc1)C(F)(F)F)S(=O)(=O)C1CCOCC1] | (R)-N-Ethyl-N-(2,2,2-trifluoro-1-(4- methoxyphenyl)ethyl)tetrahydro-2H-pyran- 4-sulfonamide |
| 128 | 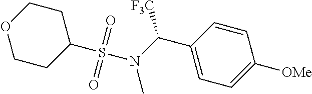 [SMILES: COc1ccc([C@H](N(C)S(=O)(=O)C2CCOCC2)C(F)(F)F)cc1] | (S)-N-Methyl-N-(2,2,2-trifluoro-1-(4- methoxyphenyl)ethyl)tetrahydro-2H-pyran- 4-sulfonamide |
| 129 | 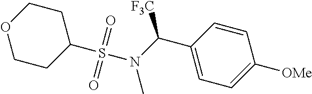 [SMILES: COc1ccc([C@@H](N(C)S(=O)(=O)C2CCOCC2)C(F)(F)F)cc1] | (R)-N-Methyl-N-(2,2,2-trifluoro-1-(4- methoxyphenyl)ethyl)tetrahydro-2H-pyran- 4-sulfonamide |
| 130 | 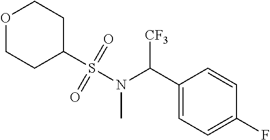 [SMILES: CN(C(c1ccc(F)cc1)C(F)(F)F)S(=O)(=O)C1CCOCC1] | N-Methyl-N-(2,2,2-trifluoro-1-(4- fluorophenyl)ethyl)tetrahydro-2H-pyran-4- sulfonamide |
| 131 | 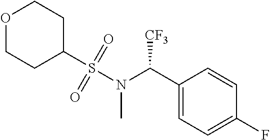 | (S)-N-Methyl-N-(2,2,2-trifluoro-1-(4- fluorophenyl)ethyl)tetrahydro-2H-pyran-4- sulfonamide |
| 132 | 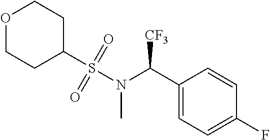 | (R)-N-Methyl-N-(2,2,2-trifluoro-1-(4- fluorophenyl)ethyl)tetrahydro-2H-pyran-4- sulfonamide |
| 133 | 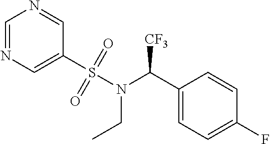 | (R)-N-Ethyl-N-(2,2,2-trifluoro-1-(4- fluorophenyl)ethyl)pyrimidine-5-sulfonamide |
| 134 | 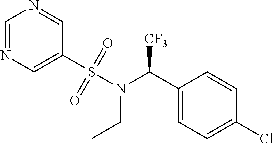 | (R)-N-(1-(4-chlorophenyl)-2,2,2- trifluoroethyl)-N-ethylpyrimidine-5- sulfonamide |
| 135 | 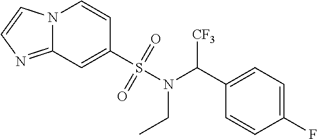 [SMILES: CCN(C(c1ccc(F)cc1)C(F)(F)F)S(=O)(=O)c1ccn2ccnc2c1] | N-Ethyl-N-(2,2,2-trifluoro-1-(4- fluorophenyl)ethyl)imidazo[1,2-a]pyridine-7- sulfonamide |
| 136 | 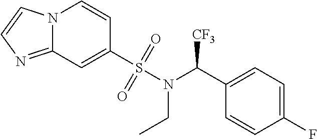 | (R)-N-Ethyl-N-(2,2,2-trifluoro-1-(4- fluorophenyl)ethyl)imidazo[1,2-a]pyridine-7- sulfonamide |
| 137 | 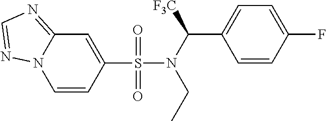 [SMILES: CCN([C@H](c1ccc(F)cc1)C(F)(F)F)S(=O)(=O)c1ccn2ncnc2c1] | (R)-N-Ethyl-N-(2,2,2-trifluoro-1-(4- fluorophenyl)ethyl)-[1,2,4]triazolo[1,5- a]pyridine-7-sulfonamide |
| 138 | 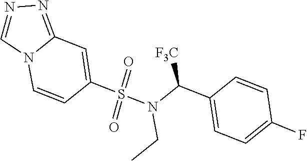 [SMILES: CCN([C@H](c1ccc(F)cc1)C(F)(F)F)S(=O)(=O)c1ccn2cnnc2c1] | (R)-N-Ethyl-N-(2,2,2-trifluoro-1-(4- fluorophenyl)ethyl)-[1,2,4]triazolo[4,3- a]pyridine-7-sulfonamide |
| 139 | 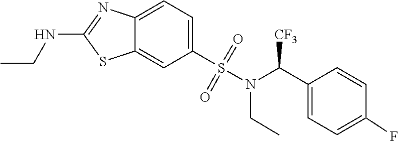 [SMILES: CCNc1nc2ccc(S(=O)(=O)N(CC)[C@H](c3ccc(F)cc3)C(F)(F)F)cc2s1] | (R)-N-ethyl-2-(ethylamino)-N-(2,2,2-trifluoro- 1-(4-fluorophenyl)ethyl)benzo[d]thiazole-6- sulfonamide |
| 140 | 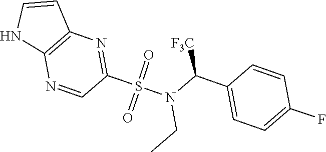 [SMILES: CCN([C@H](c1ccc(F)cc1)C(F)(F)F)S(=O)(=O)c1cnc2[nH]ccc2n1] | (R)-N-ethyl-N-(2,2,2-trifluoro-1-(4- fluorophenyl)ethyl)-5H-pyrrolo[2,3- b]pyrazine-2-sulfonamide |
| 141 | 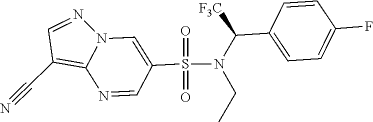 [SMILES: CCN([C@H](c1ccc(F)cc1)C(F)(F)F)S(=O)(=O)c1cnc2c(C#N)cnn2c1] | (R)-3-cyano-N-ethyl-N-(2,2,2-trifluoro-1-(4- fluorophenyl)ethyl)pyrazolo[1,5- a]pyrimidine-6-sulfonamide |
| 142 | 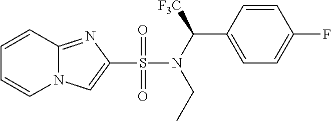 [SMILES: CCN([C@H](c1ccc(F)cc1)C(F)(F)F)S(=O)(=O)c1cn2ccccc2n1] | (R)-N-ethyl-N-(2,2,2-trifluoro-1-(4- fluorophenyl)ethyl)imidazo[1,2-a]pyridine-2- sulfonamide |
| 143 | 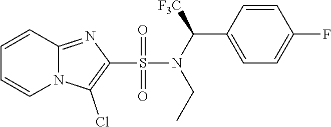 [SMILES: CCN([C@H](c1ccc(F)cc1)C(F)(F)F)S(=O)(=O)c1nc2ccccn2c1Cl] | (R)-3-chloro-N-ethyl-N-(2,2,2-trifluoro-1-(4- fluorophenyl)ethyl)imidazo[1,2-a]pyridine-2- sulfonamide |
| 144 | 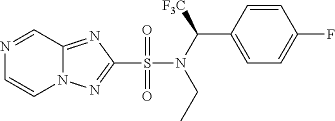 [SMILES: CCN([C@H](c1ccc(F)cc1)C(F)(F)F)S(=O)(=O)c1nc2cnccn2n1] | (R)-N-ethyl-N-(2,2,2-trifluoro-1-(4- fluorophenyl)ethyl)-[1,2,4]triazolo[1,5- a]pyrazine-2-sulfonamide |
| 145 | 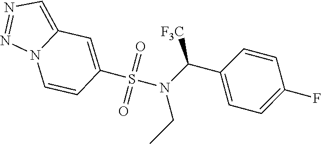 [SMILES: CCN([C@H](c1ccc(F)cc1)C(F)(F)F)S(=O)(=O)c1ccn2nncc2c1] | (R)-N-ethyl-N-(2,2,2-trifluoro-1-(4- fluorophenyl)ethyl)-[1,2,3]triazolo[1,5- a]pyridine-5-sulfonamide |
| 146 | 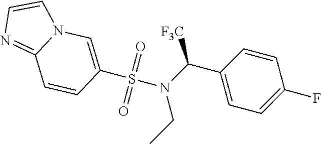 [SMILES: CCN([C@H](c1ccc(F)cc1)C(F)(F)F)S(=O)(=O)c1ccc2nccn2c1] | (R)-N-ethyl-N-(2,2,2-trifluoro-1-(4- fluorophenyl)ethyl)imidazo[1,2-a]pyridine-6- sulfonamide |
| 147 | 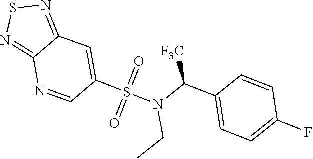 [SMILES: CCN([C@H](c1ccc(F)cc1)C(F)(F)F)S(=O)(=O)c1cnc2nsnc2c1] | (R)-N-ethyl-N-(2,2,2-trifluoro-1-(4- fluorophenyl)ethyl)-[1,2,5]thiadiazolo[3,4- b]pyridine-6-sulfonamide |
| 148 | 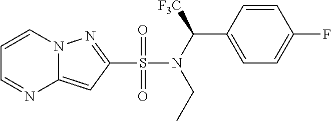 [SMILES: CCN([C@H](c1ccc(F)cc1)C(F)(F)F)S(=O)(=O)c1cc2ncccn2n1] | (R)-N-ethyl-N-(2,2,2-trifluoro-1-(4- fluorophenyl)ethyl)pyrazolo[1,5- a]pyrimidine-2-sulfonamide |
| 149 | 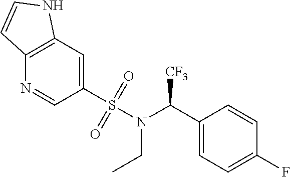 | (R)-N-ethyl-N-(2,2,2-trifluoro-1-(4- fluorophenyl)ethyl)-1H-pyrrolo[3,2- b]pyridine-6-sulfonamide |
| 150 | 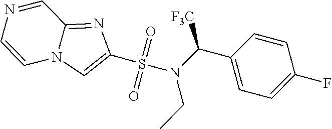 [SMILES: CCN([C@H](c1ccc(F)cc1)C(F)(F)F)S(=O)(=O)c1cn2ccncc2n1] | (R)-N-ethyl-N-(2,2,2-trifluoro-1-(4- fluorophenyl)ethyl)imidazo[1,2-a]pyrazine-2- sulfonamide |
| 151 | 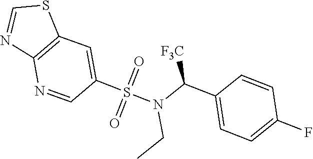 [SMILES: CCN([C@H](c1ccc(F)cc1)C(F)(F)F)S(=O)(=O)c1cnc2ncsc2c1] | (R)-N-ethyl-N-(2,2,2-trifluoro-1-(4- fluorophenyl)ethyl)thiazolo[4,5-b]pyridine-6- sulfonamide |
| 152 | 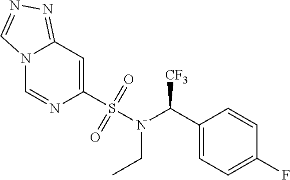 | (R)-N-ethyl-N-(2,2,2-trifluoro-1-(4- fluorophenyl)ethyl)-[1,2,4]triazolo[4,3- c]pyrimidine-7-sulfonamide |
| 153 | 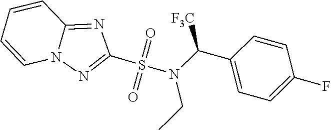 [SMILES: CCN([C@H](c1ccc(F)cc1)C(F)(F)F)S(=O)(=O)c1nc2ccccn2n1] | (R)-N-ethyl-N-(2,2,2-trifluoro-1-(4- fluorophenyl)ethyl)-[1,2,4]triazolo[1,5- a]pyridine-2-sulfonamide |
| 154 | 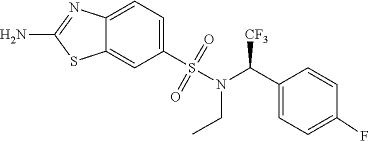 [SMILES: CCN([C@H](c1ccc(F)cc1)C(F)(F)F)S(=O)(=O)c1ccc2nc(N)sc2c1] | (R)-2-amino-N-ethyl-N-(2,2,2-trifluoro-1-(4- fluorophenyl)ethyl)benzo[d]thiazole-6- sulfonamide |
| 155 | 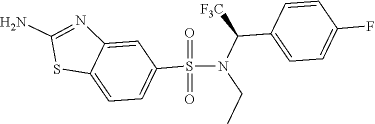 [SMILES: CCN([C@H](c1ccc(F)cc1)C(F)(F)F)S(=O)(=O)c1ccc2sc(N)nc2c1] | (R)-2-amino-N-ethyl-N-(2,2,2-trifluoro-1-(4- fluorophenyl)ethyl)benzo[d]thiazole-5- sulfonamide |
| 156 | 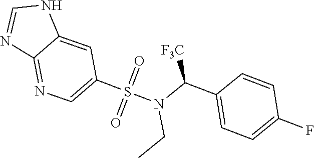 [SMILES: CCN([C@H](c1ccc(F)cc1)C(F)(F)F)S(=O)(=O)c1cnc2nc[nH]c2c1] | (R)-N-ethyl-N-(2,2,2-trifluoro-1-(4- fluorophenyl)ethyl)-1H-imidazo[4,5- b]pyridine-6-sulfonamide |
| 157 | 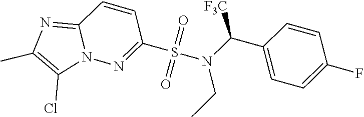 [SMILES: CCN([C@H](c1ccc(F)cc1)C(F)(F)F)S(=O)(=O)c1ccc2nc(C)c(Cl)n2n1] | (R)-3-chloro-N-ethyl-2-methyl-N-(2,2,2- trifluoro-1-(4-fluorophenyl)ethyl)imidazo[1,2- b]pyridazine-6-sulfonamide |
| 158 | 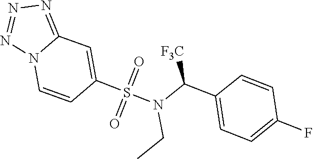 [SMILES: CCN([C@H](c1ccc(F)cc1)C(F)(F)F)S(=O)(=O)c1ccn2nnnc2c1] | (R)-N-ethyl-N-(2,2,2-trifluoro-1-(4- fluorophenyl)ethyl)tetrazolo[1,5-a]pyridine- 7-sulfonamide |
| 159 | 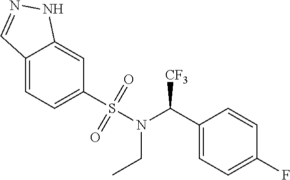 | (R)-N-ethyl-N-(2,2,2-trifluoro-1-(4- fluorophenyl)ethyl)-1H-indazole-6- sulfonamide |
| 160 | 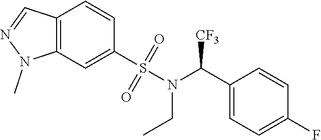 | (R)-N-ethyl-1-methyl-N-(2,2,2-trifluoro-1-(4- fluorophenyl)ethyl)-1H-indazole-6- sulfonamide |
| 161 | 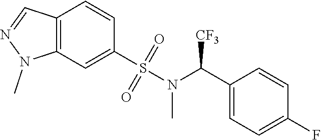 | (R)-N-methyl-1-methyl-N-(2,2,2-trifluoro-1- (4-fluorophenyl)ethyl)-1H-indazole-6- sulfonamide |
| 162 | 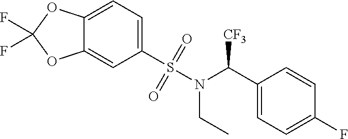 | (R)-N-ethyl-2,2-difluoro-N-(2,2,2-trifluoro-1- (4-fluorophenyl)ethyl)benzo[d][1,3]dioxole- 5-sulfonamide |
| 163 | 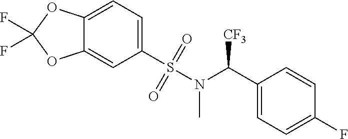 | (R)-N-methyl-2,2-difluoro-N-(2,2,2-trifluoro- 1-(4- fluorophenyl)ethyl)benzo[d][1,3]dioxole-5- sulfonamide |
| 164 | 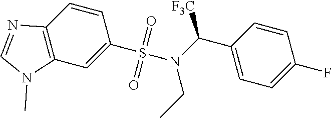 [SMILES: CCN([C@H](c1ccc(F)cc1)C(F)(F)F)S(=O)(=O)c1ccc2ncn(C)c2c1] | (R)-N-ethyl-1-methyl-N-(2,2,2-trifluoro-1-(4- fluorophenyl)ethyl)-1H-benzo[d]imidazole-6- sulfonamide |
| 165 | 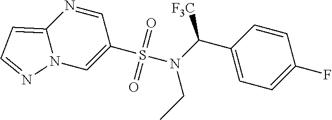 [SMILES: CCN([C@H](c1ccc(F)cc1)C(F)(F)F)S(=O)(=O)c1cnc2ccnn2c1] | (R)-N-ethyl-N-(2,2,2-trifluoro-1-(4- fluorophenyl)ethyl)pyrazolo[1,5- a]pyrimidine-6-sulfonamide |
| 166 | 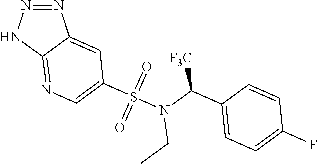 [SMILES: CCN([C@H](c1ccc(F)cc1)C(F)(F)F)S(=O)(=O)c1cnc2[nH]nnc2c1] | (R)-N-ethyl-N-(2,2,2-trifluoro-1-(4- fluorophenyl)ethyl)-1H-[1,2,3]triazolo[4,5- b]pyridine-6-sulfonamide |
| 167 | 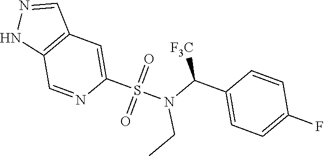 [SMILES: CCN([C@H](c1ccc(F)cc1)C(F)(F)F)S(=O)(=O)c1cc2cn[nH]c2cn1] | (R)-N-ethyl-N-(2, 2, 2-trifluoro-1-(4- fluorophenyl) ethyl)-1H-pyrazolo[3,4- c]pyridine-5-sulfonamide |
| 168 | 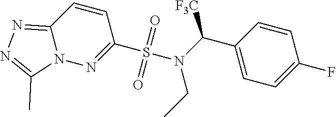 [SMILES: CCN([C@H](c1ccc(F)cc1)C(F)(F)F)S(=O)(=O)c1ccc2nnc(C)n2n1] | (R)-N-ethyl-3-methyl-N-(2,2,2-trifluoro-1-(4- fluorophenyl)ethyl)-[1,2,4]triazolo[4,3- b]pyridazine-6-sulfonamide |
| 169 | 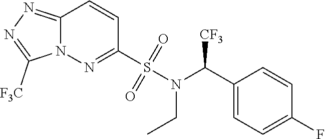 | (R)-N-ethyl-N-(2,2,2-trifluoro-1-(4- fluorophenyl)ethyl)-3-(trifluoromethyl)- [1,2,4]triazolo[4,3-b]pyridazine-6- sulfonamide |
| 170 | 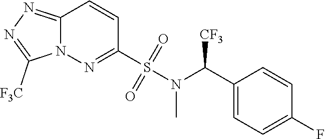 | (R)-N-methyl-N-(2,2,2-trifluoro-1-(4- fluorophenyl)ethyl)-3-(trifluoromethyl)- [1,2,4]triazolo[4,3-b]pyridazine-6- sulfonamide |
| 171 | 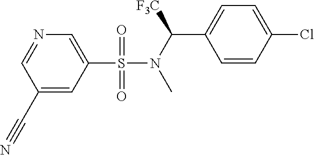 [SMILES: CN([C@H](c1ccc(Cl)cc1)C(F)(F)F)S(=O)(=O)c1cncc(C#N)c1] | (R)-N-(1-(4-chlorophenyl)-2,2,2- trifluoroethyl)-5-cyano-N-methylpyridine-3- sulfonamide |
| 172 | 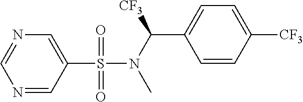 | (R)-N-methyl-N-(2,2,2-trifluoro-1-(4- (trifluoromethyl)phenyl) ethyl)pyrimidine-5- sulfonamide |
| 173 | 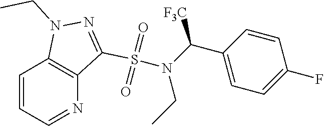 [SMILES: CCN([C@H](c1ccc(F)cc1)C(F)(F)F)S(=O)(=O)c1nn(CC)c2cccnc12] | (R)-N,1-diethyl-N-(2,2,2-trifluoro-1-(4- fluorophenyl)ethyl)-1H-pyrazolo[4,3- b]pyridine-3-sulfonamide |
| 174 | 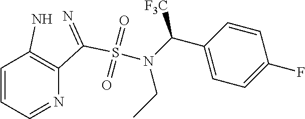 [SMILES: CCN([C@H](c1ccc(F)cc1)C(F)(F)F)S(=O)(=O)C(=N)c1ncccc1N] | (R)-N-ethyl-N-(2,2,2-trifluoro-1-(4- fluorophenyl)ethyl)-1H-pyrazolo[4,3- b]pyridine-3-sulfonamide |
| 175 | 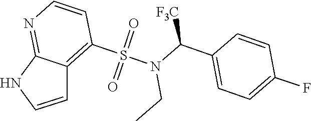 [SMILES: CCN([C@H](c1ccc(F)cc1)C(F)(F)F)S(=O)(=O)c1ccnc2[nH]ccc12] | (R)-N-ethyl-N-(2,2,2-trifluoro-1-(4- fluorophenyl)ethyl)-1H-pyrrolo[2,3- b]pyridine-4-sulfonamide |
| 176 | 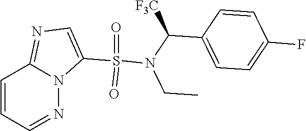 [SMILES: CCN([C@H](c1ccc(F)cc1)C(F)(F)F)S(=O)(=O)c1cnc2cccnn12] | (R)-N-ethyl-N-(2,2,2-trifluoro-1-(4- fluorophenyl)ethyl)imidazo[1,2-b]pyridazine- 3-sulfonamide |
| 177 | 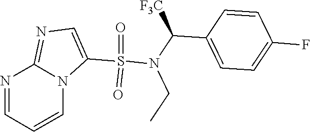 [SMILES: CCN([C@H](c1ccc(F)cc1)C(F)(F)F)S(=O)(=O)c1cnc2ncccn12] | (R)-N-ethyl-N-(2,2,2-trifluoro-1-(4- fluorophenyl)ethyl)imidazo[1,2-a]pyrimidine- 3-sulfonamide |
| 178 | 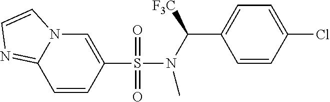 [SMILES: CN([C@H](c1ccc(Cl)cc1)C(F)(F)F)S(=O)(=O)c1ccc2nccn2c1] | (R)-N-(1-(4-chlorophenyl)-2,2,2- trifluoroethyl)-N-methylimidazo[1,2- a]pyridine-6-sulfonamide |
| 179 | 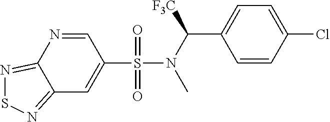 [SMILES: CN([C@H](c1ccc(Cl)cc1)C(F)(F)F)S(=O)(=O)c1cnc2nsnc2c1] | (R)-N-(1-(4-chlorophenyl)-2,2,2- trifluoroethyl)-N-methyl- [1,2,5]thiadiazolo[3,4-b]pyridine-6- sulfonamide |
| 180 | 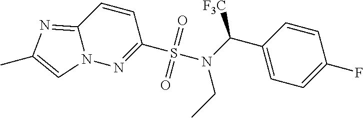 [SMILES: CCN([C@H](c1ccc(F)cc1)C(F)(F)F)S(=O)(=O)c1ccc2nc(C)cn2n1] | (R)-N-ethyl-2-methyl-N-(2,2,2-trifluoro-1-(4- fluorophenyl)ethyl)imidazo[1,2-b]pyridazine- 6-sulfonamide |
| 181 | 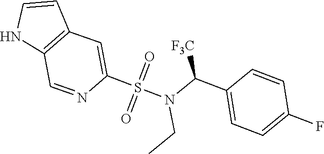 [SMILES: CCN([C@H](c1ccc(F)cc1)C(F)(F)F)S(=O)(=O)c1cc2cc[nH]c2cn1] | (R)-N-ethyl-N-(2,2,2-trifluoro-1-(4- fluorophenyl)ethyl)-1H-pyrrolo[2,3- c]pyridine-5-sulfonamide |
| 182 | 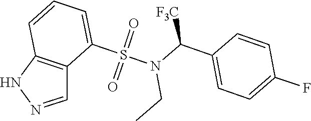 [SMILES: CCN([C@H](c1ccc(F)cc1)C(F)(F)F)S(=O)(=O)c1cccc2[nH]ncc12] | (R)-N-ethyl-N-(2,2,2-trifluoro-1-(4- fluorophenyl)ethyl)-1H-indazole-4- sulfonamide |
| 183 | 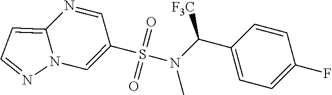 [SMILES: CN([C@H](c1ccc(F)cc1)C(F)(F)F)S(=O)(=O)c1cnc2ccnn2c1] | (R)-N-methyl-N-(2,2,2-trifluoro-1-(4- fluorophenyl)ethyl)pyrazolo[1,5- a]pyrimidine-6-sulfonamide |
| 184 | 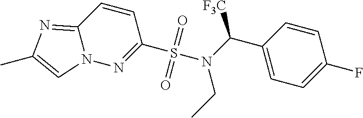 [SMILES: CCN([C@H](c1ccc(F)cc1)C(F)(F)F)S(=O)(=O)c1ccc2nc(C)cn2n1] | (R)-N-ethyl-2-methyl-N-(2,2,2-trifluoro-1-(4- fluorophenyl)ethyl)imidazo[1,2-b]pyridazine- 6-sulfonamide |
| 185 | 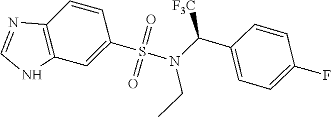 [SMILES: CCN([C@H](c1ccc(F)cc1)C(F)(F)F)S(=O)(=O)c1ccc2nc[nH]c2c1] | (R)-N-ethyl-N-(2,2,2-trifluoro-1-(4- fluorophenyl)ethyl)-1H-benzo[d]imidazole-6- sulfonamide |
| 186 | 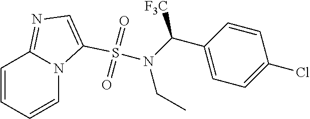 [SMILES: CCN([C@H](c1ccc(Cl)cc1)C(F)(F)F)S(=O)(=O)c1cnc2ccccn12] | (R)-N-ethyl-N-(2,2,2-trifluoro-1-(4- fluorophenyl)ethyl)imidazo[1,2-a]pyridine-3- sulfonamide |
| 187 | 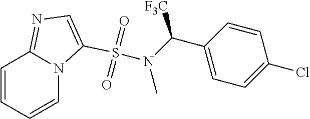 [SMILES: CN([C@H](c1ccc(Cl)cc1)C(F)(F)F)S(=O)(=O)c1cnc2ccccn12] | (R)-N-(1-(4-chlorophenyl)-2,2,2- trifluoroethyl)-N-methylimidazo[1,2- a]pyridine-3-sulfonamide |
| 188 | 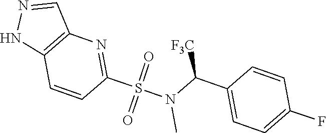 [SMILES: CN([C@H](c1ccc(F)cc1)C(F)(F)F)S(=O)(=O)c1ccc2[nH]ncc2n1] | (R)-N-ethyl-N-(2,2,2-trifluoro-1-(4- fluorophenyl)ethyl)-1H-pyrazolo[4,3- b]pyridine-5-sulfonamide |
| 189 | 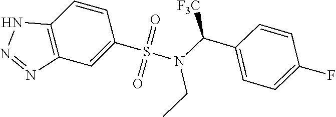 [SMILES: CCN([C@H](c1ccc(F)cc1)C(F)(F)F)S(=O)(=O)c1ccc2[nH]nnc2c1] | (R)-N-ethyl-N-(2,2,2-trifluoro-1-(4- fluorophenyl)ethyl)-1H- benzo[d][1,2,3]triazole-5-sulfonamide |
| 190 | 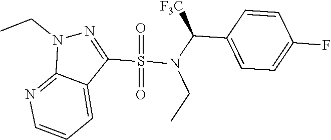 [SMILES: CCN([C@H](c1ccc(F)cc1)C(F)(F)F)S(=O)(=O)c1nn(CC)c2ncccc12] | (R)-N,1-diethyl-N-(2,2,2-trifluoro-1-(4- fluorophenyl)ethyl)-1H-pyrazolo[3,4- b]pyridine-3-sulfonamide |
| 191 | 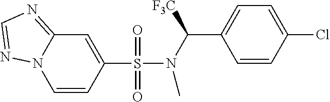 [SMILES: CN([C@H](c1ccc(Cl)cc1)C(F)(F)F)S(=O)(=O)c1ccn2ncnc2c1] | (R)-N-(1-(4-chlorophenyl)-2,2,2- trifluoroethyl)-N-methyl-[1,2,4]triazolo[1,5- a]pyridine-7-sulfonamide |
| 192 | 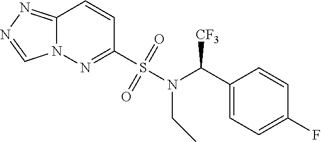 | (R)-N-ethyl-N-(2,2,2-trifluoro-1-(4- fluorophenyl)ethyl)-[1,2,4]triazolo[4,3- b]pyridazine-6-sulfonamide |
| 193 | 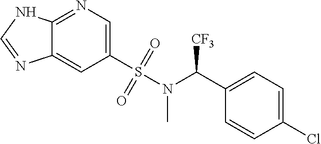 | (R)-N-(1-(4-chlorophenyl)-2,2,2- trifluoroethyl)-N-methyl-3H-imidazo[4,5- b]pyridine-6-sulfonamide |
| 194 | 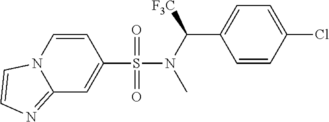 [SMILES: CN([C@H](c1ccc(Cl)cc1)C(F)(F)F)S(=O)(=O)c1ccn2ccnc2c1] | (R)-N-(1-(4-chlorophenyl)-2,2,2- trifluoroethyl)-N-methylimidazo[1,2- a]pyridine-7-sulfonamide |
| 195 | 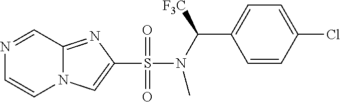 [SMILES: CN([C@H](c1ccc(Cl)cc1)C(F)(F)F)S(=O)(=O)c1cn2ccncc2n1] | (R)-N-(1-(4-chlorophenyl)-2,2,2- trifluoroethyl)-N-methylimidazo[1,2- a]pyrazine-2-sulfonamide |
| 196 | 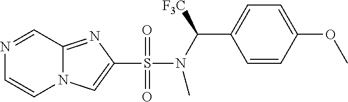 [SMILES: COc1ccc([C@@H](N(C)S(=O)(=O)c2cn3ccncc3n2)C(F)(F)F)cc1] | (R)-N-methyl-N-(2,2,2-trifluoro-1-(4- methoxyphenyl)ethyl)imidazo[1,2- a]pyrazine-2-sulfonamide |
| 197 | 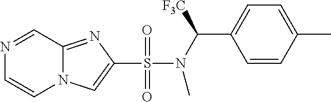 [SMILES: Cc1ccc([C@@H](N(C)S(=O)(=O)c2cn3ccncc3n2)C(F)(F)F)cc1] | (R)-N-methyl-N-(2,2,2-trifluoro-1-(p- tolyl)ethyl)imidazo[1,2-a]pyrazine-2- sulfonamide |
| 198 | 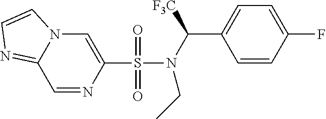 [SMILES: CCN([C@H](c1ccc(F)cc1)C(F)(F)F)S(=O)(=O)c1cn2ccnc2cn1] | (R)-N-ethyl-N-(2,2,2-trifluoro-1-(4- fluorophenyl)ethyl)imidazo[1,2-a]pyrazine-6- sulfonamide |
| 199 | 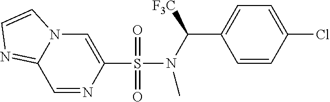 [SMILES: CN([C@H](c1ccc(Cl)cc1)C(F)(F)F)S(=O)(=O)c1cn2ccnc2cn1] | (R)-N-(1-(4-chlorophenyl)-2,2,2- trifluoroethyl)-N-methylimidazo[1,2- a]pyrazine-6-sulfonamide |
| 200 | 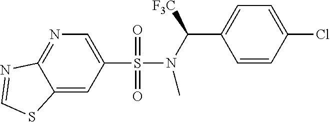 [SMILES: CN([C@H](c1ccc(Cl)cc1)C(F)(F)F)S(=O)(=O)c1cnc2ncsc2c1] | (R)-N-(1-(4-chlorophenyl)-2,2,2- trifluoroethyl)-N-methylthiazolo[4,5- b]pyridine-6-sulfonamide |
| 201 | 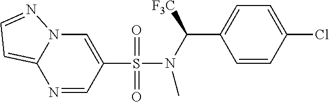 [SMILES: CN([C@H](c1ccc(Cl)cc1)C(F)(F)F)S(=O)(=O)c1cnc2ccnn2c1] | (R)-N-(1-(4-chlorophenyl)-2,2,2- trifluoroethyl)-N-methylpyrazolo[1,5- a]pyrimidine-6-sulfonamide |
| 202 | 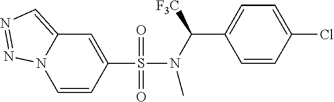 [SMILES: CN([C@H](c1ccc(Cl)cc1)C(F)(F)F)S(=O)(=O)c1ccn2nncc2c1] | (R)-N-(1-(4-chlorophenyl)-2,2,2- trifluoroethyl)-N-methyl-[1,2,3]triazolo[1,5- a]pyridine-5-sulfonamide |
| 203 | 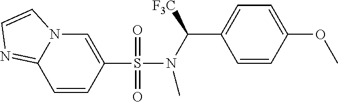 [SMILES: COc1ccc([C@@H](N(C)S(=O)(=O)c2ccc3nccn3c2)C(F)(F)F)cc1] | (R)-N-methyl-N-(2,2,2-trifluoro-1-(4- methoxyphenyl)ethyl)imidazo[1,2- a]pyridine-6-sulfonamide |
| 204 | 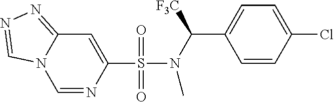 [SMILES: CN([C@H](c1ccc(Cl)cc1)C(F)(F)F)S(=O)(=O)c1cc2nncn2cn1] | (R)-N-(1-(4-chlorophenyl)-2,2,2- trifluoroethyl)-N-methyl-[1,2,4]triazolo[4,3- c]pyrimidine-7-sulfonamide |
| 205 | 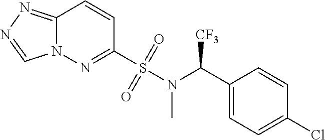 | (R)-N-(1-(4-chlorophenyl)-2,2,2- trifluoroethyl)-N-methyl-[1,2,4]triazolo[4,3- b]pyridazine-6-sulfonamide |
| 206 | 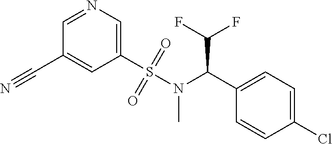 [SMILES: CN([C@H](c1ccc(Cl)cc1)C(F)F)S(=O)(=O)c1cncc(C#N)c1] | (R)-N-(1-(4-chlorophenyl)-2,2-difluoroethyl)- 5-cyano-N-methylpyridine-3-sulfonamide |
| 207 | 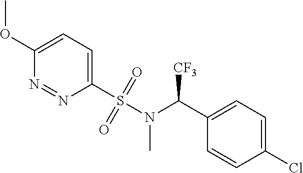 | (R)-N-(1-(4-chlorophenyl)-2,2,2- trifluoroethyl)-6-methoxy-N- methylpyridazine-3-sulfonamide |
| 208 | 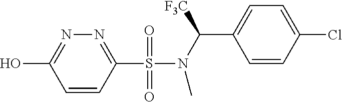 [SMILES: CN([C@H](c1ccc(Cl)cc1)C(F)(F)F)S(=O)(=O)c1ccc(O)nn1] | (R)-N-(1-(4-chlorophenyl)-2,2,2- trifluoroethyl)-6-hydroxy-N- methylpyridazine-3-sulfonamide |
| 209 | 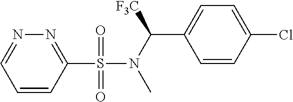 [SMILES: CN([C@H](c1ccc(Cl)cc1)C(F)(F)F)S(=O)(=O)c1cccnn1] | (R)-N-(1-(4-chlorophenyl)-2,2,2- trifluoroethyl)-N-methylpyridazine-3- sulfonamide |
| 210 | 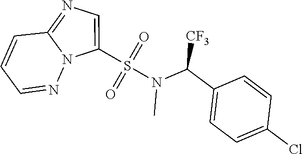 [SMILES: CN([C@H](c1ccc(Cl)cc1)C(F)(F)F)S(=O)(=O)c1cnc2cccnn12] | (R)-N-(1-(4-chlorophenyl)-2,2,2- trifluoroethyl)-N-methylimidazo[1,2- b]pyridazine-3-sulfonamide |
| 211 | 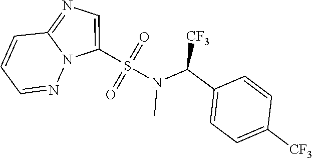 [SMILES: CN([C@H](c1ccc(C(F)(F)F)cc1)C(F)(F)F)S(=O)(=O)c1cnc2cccnn12] | (R)-N-methyl-N-(2,2,2-trifluoro-1-(4- (trifluoromethyl)phenyl)ethyl)imidazo[1,2- b]pyridazine-3-sulfonamide |
| 212 | 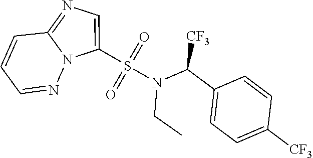 [SMILES: CCN([C@H](c1ccc(C(F)(F)F)cc1)C(F)(F)F)S(=O)(=O)c1cnc2cccnn12] | (R)-N-ethyl-N-(2,2,2-trifluoro-1-(4- (trifluoromethyl)phenyl)ethyl)imidazo[1,2- b]pyridazine-3-sulfonamide |
| 213 | 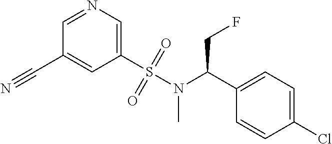 [SMILES: CN([C@@H](CF)c1ccc(Cl)cc1)S(=O)(=O)c1cncc(C#N)c1] | (R)-N-(1-(4-chlorophenyl)-2-fluoroethyl)-5- cyano-N-methylpyridine-3-sulfonamide |
| 214 | 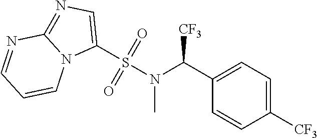 [SMILES: CN([C@H](c1ccc(C(F)(F)F)cc1)C(F)(F)F)S(=O)(=O)c1cnc2ncccn12] | (R)-N-methyl-N-(2,2,2-trifluoro-1-(4- (trifluoromethyl)phenyl)ethyl)imidazo[1,2- a]pyrimidine-3-sulfonamide |
| 215 | 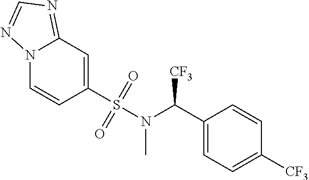 [SMILES: CN([C@H](c1ccc(C(F)(F)F)cc1)C(F)(F)F)S(=O)(=O)c1ccn2ncnc2c1] | (R)-N-methyl-N-(2,2,2-trifluoro-1-(4- (trifluoromethyl)phenyl)ethyl)- [1,2,4]triazolo[1,5-a]pyridine-7-sulfonamide |
| 216 | 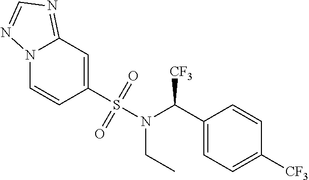 [SMILES: CCN([C@H](c1ccc(C(F)(F)F)cc1)C(F)(F)F)S(=O)(=O)c1ccn2ncnc2c1] | (R)-N-ethyl-N-(2,2,2-trifluoro-1-(4- (trifluoromethyl)phenyl)ethyl)- [1,2,4]triazolo[1,5-a]pyridine-7-sulfonamide |
| 217 | 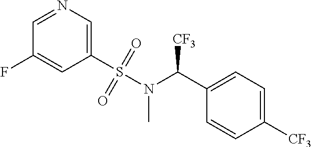 [SMILES: CN([C@H](c1ccc(C(F)(F)F)cc1)C(F)(F)F)S(=O)(=O)c1cncc(F)c1] | (R)-5-fluoro-N-methyl-N-(2,2,2-trifluoro-1-(4- (trifluoromethyl)phenyl)ethyl)pyridine-3- sulfonamide |
| 218 | 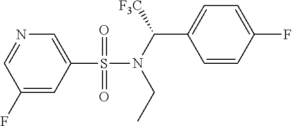 [SMILES: CCN([C@@H](c1ccc(F)cc1)C(F)(F)F)S(=O)(=O)c1cncc(F)c1] | (S)-N-ethyl-5-fluoro-N-(2,2,2-trifluoro-1-(4- fluorophenyl)ethyl)pyridine-3-sulfonamide |
| 219 | 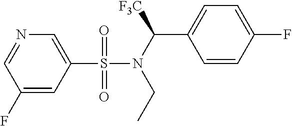 [SMILES: CCN([C@H](c1ccc(F)cc1)C(F)(F)F)S(=O)(=O)c1cncc(F)c1] | (R)-N-ethyl-5-fluoro-N-(2,2,2-trifluoro-1-(4- fluorophenyl)ethyl)pyridine-3-sulfonamide |
| 220 | 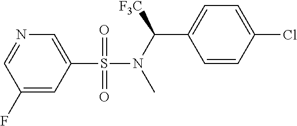 [SMILES: CN([C@H](c1ccc(Cl)cc1)C(F)(F)F)S(=O)(=O)c1cncc(F)c1] | (R)-N-(1-(4-chlorophenyl)-2,2,2- trifluoroethyl)-5-fluoro-N-methylpyridine-3- sulfonamide |
| 221 | 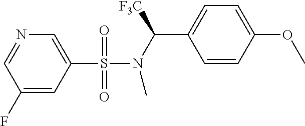 [SMILES: COc1ccc([C@@H](N(C)S(=O)(=O)c2cncc(F)c2)C(F)(F)F)cc1] | (R)-5-fluoro-N-methyl-N-(2,2,2-trifluoro-1-(4- methoxyphenyl)ethyl)pyridine-3- sulfonamide |
| 222 | 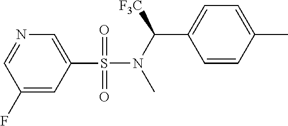 [SMILES: Cc1ccc([C@@H](N(C)S(=O)(=O)c2cncc(F)c2)C(F)(F)F)cc1] | (R)-5-fluoro-N-methyl-N-(2,2,2-trifluoro-1-(p- tolyl)ethyl)pyridine-3-sulfonamide |
| 223 | 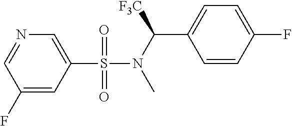 [SMILES: CN([C@H](c1ccc(F)cc1)C(F)(F)F)S(=O)(=O)c1cncc(F)c1] | (R)-5-fluoro-N-methyl-N-(2,2,2-trifluoro-1-(4- fluorophenyl)ethyl)pyridine-3-sulfonamide |
| 224 | 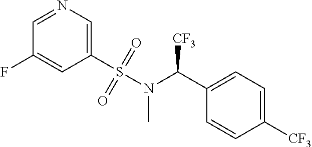 [SMILES: CN([C@H](c1ccc(C(F)(F)F)cc1)C(F)(F)F)S(=O)(=O)c1cncc(F)c1] | (R)-5-fluoro-N-methyl-N-(2,2,2-trifluoro-1-(4- (trifluoromethyl)phenyl)ethyl)pyridine-3- sulfonamide |
| 225 | 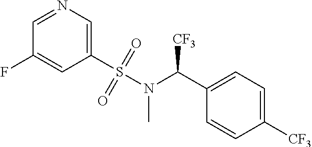 | (R)-5-cyano-N-methyl-N-(2,2,2-trifluoro-1- (p-tolyl)ethyl)pyridine-3-sulfonamide |
| 226 | 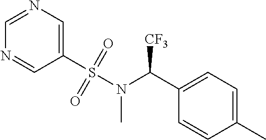 | (R)-N-methyl-N-(2,2,2-trifluoro-1-(p- tolyl)ethyl)pyrimidine-5-sulfonamide |
| 227 | 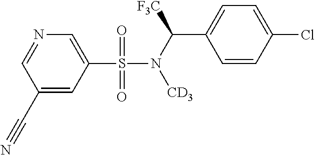 [SMILES: [2H]C([2H])([2H])N([C@H](c1ccc(Cl)cc1)C(F)(F)F)S(=O)(=O)c1cncc(C#N)c1] | (R)-N-(1-(4-chlorophenyl)-2,2,2- trifluoroethyl)-5-cyano-N-(methyl- d3)pyridine-3-sulfonamide |
| 228 | 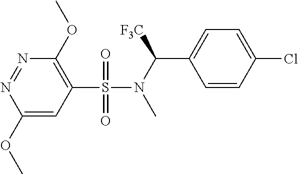 [SMILES: COc1cc(S(=O)(=O)N(C)[C@H](c2ccc(Cl)cc2)C(F)(F)F)c(OC)nn1] | (R)-N-(1-(4-chlorophenyl)-2,2,2- trifluoroethyl)-3,6-dimethoxy-N- methylpyridazine-4-sulfonamide |
| 229 | 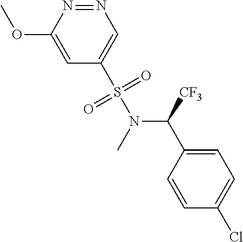 [SMILES: COc1cc(S(=O)(=O)N(C)[C@H](c2ccc(Cl)cc2)C(F)(F)F)cnn1] | (R)-N-(1-(4-chlorophenyl)-2,2,2- trifluoroethyl)-6-methoxy-N- methylpyridazine-4-sulfonamide |
| 230 | 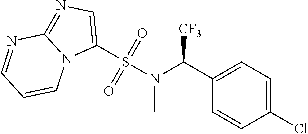 | (R)-N-(1-(4-chlorophenyl)-2,2,2- trifluoroethyl)-N-methylimidazo[1,2- a]pyrimidine-3-sulfonamide |
| 231 | 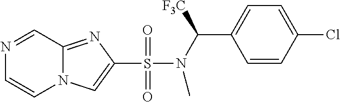 [SMILES: CN([C@H](c1ccc(Cl)cc1)C(F)(F)F)S(=O)(=O)c1cn2ccncc2n1] | (R)-N-(1-(4-chlorophenyl)-2,2,2- trifluoroethyl)-N-methylimidazo[1,2- a]pyrazine-2-sulfonamide |
| 232 | 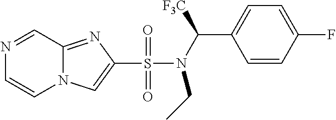 | (R)-N-ethyl-N-(2,2,2-trifluoro-1-(4- fluorophenyl)ethyl)imidazo[1,2-a]pyrazine-2- sulfonamide |
| 233 | 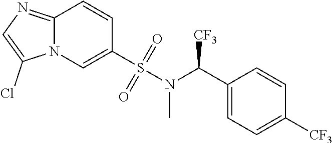 | (R)-3-chloro-N-methyl-N-(2,2,2-trifluoro-1- (4-(trifluoromethyl)phenyl)ethyl)imidazo[1,2- a]pyridine-6-sulfonamide |
| 234 | 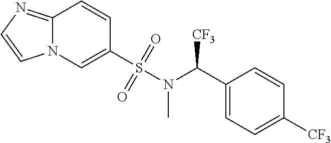 | (R)-N-methyl-N-(2,2,2-trifluoro-1-(4- (trifluoromethyl)phenyl)ethyl)imidazo[1,2- a]pyridine-6-sulfonamide |
| 235 | 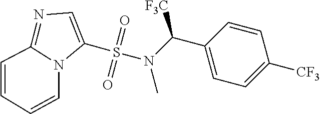 [SMILES: CN([C@H](c1ccc(C(F)(F)F)cc1)C(F)(F)F)S(=O)(=O)c1cnc2ccccn12] | (R)-N-methyl-N-(2,2,2-trifluoro-1-(4- (trifluoromethyl)phenyl)ethyl)imidazo[1,2- a]pyridine-3-sulfonamide |
| 236 | 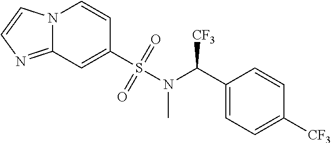 | (R)-N-methyl-N-(2,2,2-trifluoro-1-(4- (trifluoromethyl)phenyl)ethyl)imidazo[1,2- a]pyridine-7-sulfonamide |
| 237 | 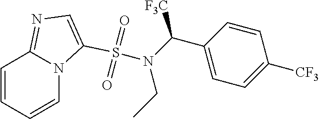 [SMILES: CCN([C@H](c1ccc(C(F)(F)F)cc1)C(F)(F)F)S(=O)(=O)c1cnc2ccccn12] | (R)-N-ethyl-N-(2,2,2-trifluoro-1-(4- (trifluoromethyl)phenyl)ethyl)imidazo[1,2- a]pyridine-3-sulfonamide |
| 238 | 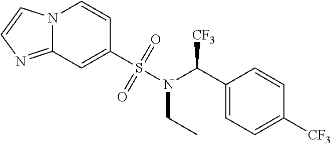 | (R)-N-methyl-N-(2,2,2-trifluoro-1-(4- (trifluoromethyl)phenyl)ethyl)imidazo[1,2- a]pyridine-7-sulfonamide |
| 239 | 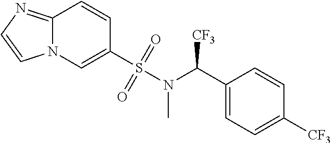 | (R)-N-ethyl-N-(2,2,2-trifluoro-1-(4- (trifluoromethyl)phenyl)ethyl)imidazo[1,2- a]pyridine-6-sulfonamidesulfonamide |
| 240 | 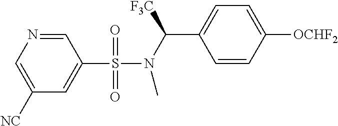 [SMILES: CN([C@H](c1ccc(OC(F)F)cc1)C(F)(F)F)S(=O)(=O)c1cncc(C#N)c1] | (R)-5-cyano-N-(1-(4- (difluoromethoxy)phenyl)-2,2,2- trifluoroethyl)-N-methylpyridine-3- sulfonamide |
| 241 | 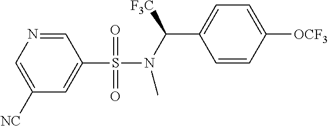 [SMILES: CN([C@H](c1ccc(OC(F)(F)F)cc1)C(F)(F)F)S(=O)(=O)c1cncc(C#N)c1] | (R)-5-cyano-N-methyl-N-(2,2,2-trifluoro-1- (4-(trifluoromethoxy)phenyl)ethyl)pyridine-3- sulfonamide |
| 242 | 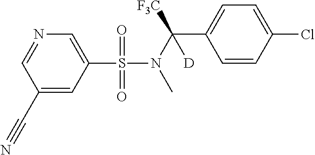 [SMILES: [2H][C@@](c1ccc(Cl)cc1)(N(C)S(=O)(=O)c1cncc(C#N)c1)C(F)(F)F] | N-(1-(4-chlorophenyl)-2,2,2-trifluoroethyl-1- d)-5-cyano-N-methylpyridine-3-sulfonamide |
| 243 | 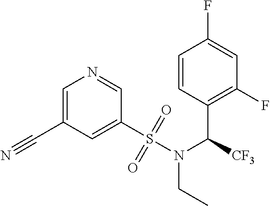 | (S)-5-cyano-N-(1-(2,4-difluorophenyl)-2,2,2- trifluoroethyl)-N-ethylpyridine-3-sulfonamide |
| 244 | 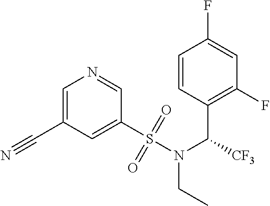 | (R)-5-cyano-N-(1-(2,4-difluorophenyl)-2,2,2- trifluoroethyl)-N-ethylpyridine-3-sulfonamide |
| 245 | 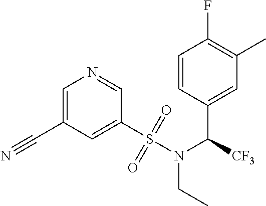 | (S)-5-cyano-N-ethyl-N-(2,2,2-trifluoro-1-(4- fluoro-3-methylphenyl)ethyl)pyridine-3- sulfonamide |
| 246 | 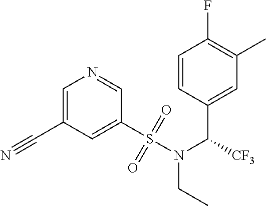 | (R)-5-cyano-N-ethyl-N-(2,2,2-trifluoro-1-(4- fluoro-3-methylphenyl)ethyl)pyridine-3- sulfonamide |
| 247 | 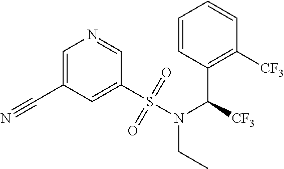 | (S)-5-cyano-N-ethyl-N-(2,2,2-trifluoro-1-(2- (trifluoromethyl)phenyl)ethyl)pyridine-3- sulfonamide |
| 248 | 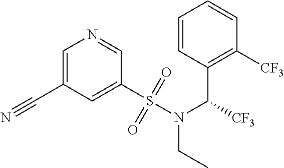 | (R)-5-cyano-N-ethyl-N-(2,2,2-trifluoro-1-(2- (trifluoromethyl)phenyl)ethyl)pyridine-3- sulfonamide |
| 249 | 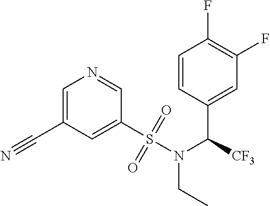 | (S)-5-cyano-N-(1-(3,4-difluorophenyl)-2,2,2- trifluoroethyl)-N-ethylpyridine-3-sulfonamide |
| 250 | 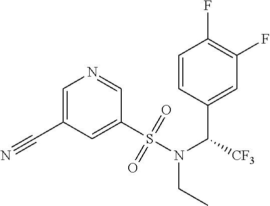 | (R)-5-cyano-N-(1-(3,4-difluorophenyl)-2,2,2- trifluoroethyl)-N-ethylpyridine-3-sulfonamide |
| 251 | 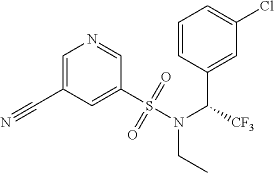 | (R)-N-(1-(3-chlorophenyl)-2,2,2- trifluoroethyl)-5-cyano-N-ethylpyridine-3- sulfonamide |
| 252 | 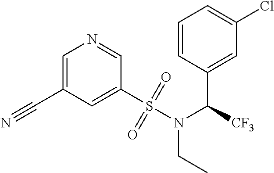 | (S)-N-(1-(3-chlorophenyl)-2,2,2- trifluoroethyl)-5-cyano-N-ethylpyridine-3- sulfonamide |
| 253 | 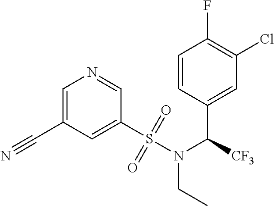 | (S)-N-(1-(3-chloro-4-fluorophenyl)-2,2,2- trifluoroethyl)-5-cyano-N-ethylpyridine-3- sulfonamide |
| 254 | 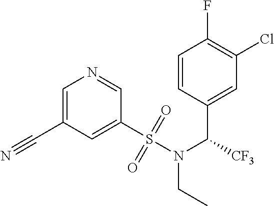 | (R)-N-(1-(3-chloro-4-fluorophenyl)-2,2,2- trifluoroethyl)-5-cyano-N-ethylpyridine-3- sulfonamide |
| 255 | 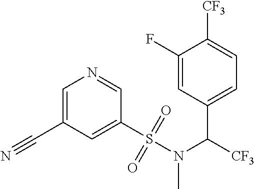 [SMILES: CN(C(c1ccc(C(F)(F)F)c(F)c1)C(F)(F)F)S(=O)(=O)c1cncc(C#N)c1] | 5-cyano-N-methyl-N-(2,2,2-trifluoro-1-(3- fluoro-4- (trifluoromethyl)phenyl)ethyl)pyridine-3- sulfonamide |
| 256 | 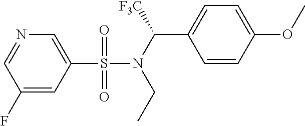 [SMILES: CCN([C@@H](c1ccc(OC)cc1)C(F)(F)F)S(=O)(=O)c1cncc(F)c1] | (S)-N-ethyl-5-fluoro-N-(2,2,2-trifluoro-1-(4- methoxyphenyl)ethyl)pyridine-3- sulfonamide |
| 257 | 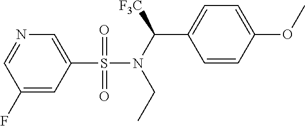 [SMILES: CCN([C@H](c1ccc(OC)cc1)C(F)(F)F)S(=O)(=O)c1cncc(F)c1] | (R)-N-ethyl-5-fluoro-N-(2,2,2-trifluoro-1-(4- methoxyphenyl)ethyl)pyridine-3- sulfonamide |
| 258 | 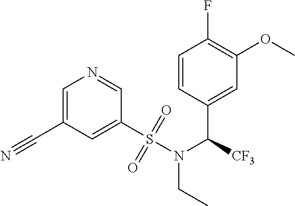 | (S)-5-cyano-N-ethyl-N-(2,2,2-trifluoro-1-(4- fluoro-3-methoxyphenyl)ethyl)pyridine-3- sulfonamide |
| 259 | 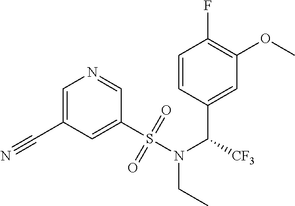 | (R)-5-cyano-N-ethyl-N-(2,2,2-trifluoro-1-(4- fluoro-3-methoxyphenyl)ethyl)pyridine-3- sulfonamide |
| 260 | 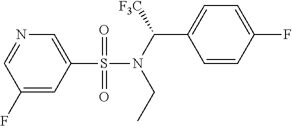 [SMILES: CCN([C@@H](c1ccc(F)cc1)C(F)(F)F)S(=O)(=O)c1cncc(F)c1] | (S)-N-ethyl-5-fluoro-N-(2,2,2-trifluoro-1-(4- fluorophenyl)ethyl)pyridine-3-sulfonamide |
| 261 | 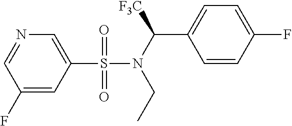 [SMILES: CCN([C@H](c1ccc(F)cc1)C(F)(F)F)S(=O)(=O)c1cncc(F)c1] | (R)-N-ethyl-5-fluoro-N-(2,2,2-trifluoro-1-(4- fluorophenyl)ethyl)pyridine-3-sulfonamide |
| 262 | 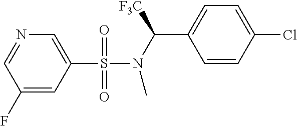 [SMILES: CN([C@H](c1ccc(Cl)cc1)C(F)(F)F)S(=O)(=O)c1cncc(F)c1] | (R)-N-(1-(4-chlorophenyl)-2,2,2- trifluoroethyl)-5-fluoro-N-methylpyridine-3- sulfonamide |
| 263 | 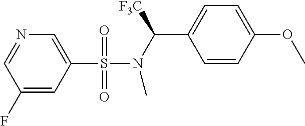 [SMILES: COc1ccc([C@@H](N(C)S(=O)(=O)c2cncc(F)c2)C(F)(F)F)cc1] | (R)-5-fluoro-N-methyl-N-(2,2,2-trifluoro-1-(4- methoxyphenyl)ethyl)pyridine-3- sulfonamide |
| 264 | 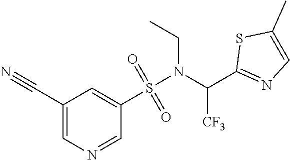 [SMILES: CCN(C(c1ncc(C)s1)C(F)(F)F)S(=O)(=O)c1cncc(C#N)c1] | 5-Cyano-N-ethyl-N-(2,2,2-trifluoro-1-(5- methylthiazol-2-yl)ethyl)pyridine-3- sulfonamide |
| 265 | 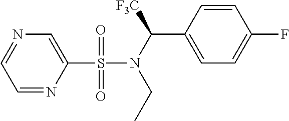 [SMILES: CCN([C@H](c1ccc(F)cc1)C(F)(F)F)S(=O)(=O)c1cnccn1] | (R)-N-ethyl-N-(2,2,2-trifluoro-1-(4- fluorophenyl)ethyl)pyrazine-2-sulfonamide |
| 266 | 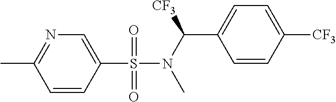 [SMILES: Cc1ccc(S(=O)(=O)N(C)[C@H](c2ccc(C(F)(F)F)cc2)C(F)(F)F)cn1] | (R)-N,6-dimethyl-N-(2,2,2-trifluoro-1-(4- (trifluoromethyl)phenyl)ethyl)pyridine-3- sulfonamide |
| 267 | 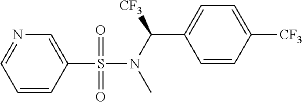 [SMILES: CN([C@H](c1ccc(C(F)(F)F)cc1)C(F)(F)F)S(=O)(=O)c1cccnc1] | (R)-N-methyl-N-(2,2,2-trifluoro-1-(4- (trifluoromethyl)phenyl)ethyl)pyridine-3- sulfonamide |
| 268 | 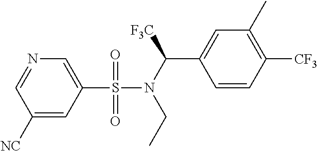 [SMILES: CCN([C@H](c1ccc(C(F)(F)F)c(C)c1)C(F)(F)F)S(=O)(=O)c1cncc(C#N)c1] | (R)-5-cyano-N-ethyl-N-(2,2,2-trifluoro-1-(3- methyl-4- (trifluoromethyl)phenyl)ethyl)pyridine-3- sulfonamide |
| 269 | 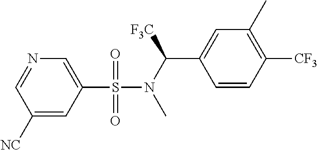 [SMILES: Cc1cc([C@@H](N(C)S(=O)(=O)c2cncc(C#N)c2)C(F)(F)F)ccc1C(F)(F)F] | (R)-5-cyano-N-methyl-N-(2,2,2-trifluoro-1- (3-methyl-4- (trifluoromethyl)phenyl)ethyl)pyridine-3- sulfonamide |
| 270 | 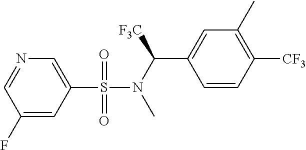 [SMILES: Cc1cc([C@@H](N(C)S(=O)(=O)c2cncc(F)c2)C(F)(F)F)ccc1C(F)(F)F] | (R)-5-fluoro-N-methyl-N-(2,2,2-trifluoro-1-(3- methyl-4- (trifluoromethyl)phenyl)ethyl)pyridine-3- sulfonamide |
| 271 | 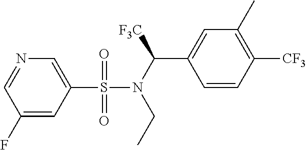 [SMILES: CCN([C@H](c1ccc(C(F)(F)F)c(C)c1)C(F)(F)F)S(=O)(=O)c1cncc(F)c1] | (R)-N-ethyl-5-fluoro-N-(2,2,2-trifluoro-1-(3- methyl-4- (trifluoromethyl)phenyl)ethyl)pyridine-3- sulfonamide |
| 272 | 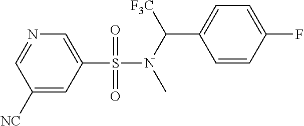 [SMILES: CN(C(c1ccc(F)cc1)C(F)(F)F)S(=O)(=O)c1cncc(C#N)c1] | 5-Cyano-N-methyl-N-(2,2,2-trifluoro-1-(4- fluorophenyl)ethyl)pyridine-3-sulfonamide |
| 273 | 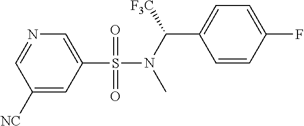 [SMILES: CN([C@@H](c1ccc(F)cc1)C(F)(F)F)S(=O)(=O)c1cncc(C#N)c1] | (S)-5-Cyano-N-methyl-N-(2,2,2-trifluoro-1- (4-fluorophenyl)ethyl)pyridine-3-sulfonamide |
| 274 | 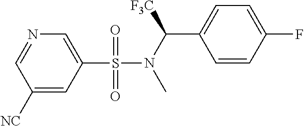 [SMILES: CN([C@H](c1ccc(F)cc1)C(F)(F)F)S(=O)(=O)c1cncc(C#N)c1] | (R)-5-Cyano-N-methyl-N-(2,2,2-trifluoro-1- (4-fluorophenyl)ethyl)pyridine-3-sulfonamide |

In another embodiment there is provided a compound selected from any one of the Examples herein, or a pharmaceutically acceptable salt thereof.

Particular compounds of the invention are those that have an pIC₅₀ of greater than 5.5, preferably those with a pIC₅₀ of 6, still more preferably those with a pIC₅₀ of 7 or more when measured in the Human Cav2.3 channel calcium-influx assay described in the Examples.

Suitably the compounds of the invention exhibit a favourable pharmacokinetic and/or pharmacodynamic profile, for example, one or more of favourable oral bioavailability, metabolic stability, plasma half-life.

### Pharmaceutical Compositions

In accordance with another aspect, the present invention provides a pharmaceutical composition comprising a compound of the invention, except that compounds A and B are not excluded, or a pharmaceutically acceptable salt thereof, and a pharmaceutically acceptable excipient.

It may be that the pharmaceutical composition comprises a compound selected from a compound according to any of formulae (I) to (XXXVIc) and compound A or compound B, or a pharmaceutically acceptable salt thereof.

It may be that the pharmaceutical composition comprises a compound selected from a compound according to any of formulae (I) to (XXXVIc), or a pharmaceutically acceptable salt thereof, with the proviso that and compound A and compound B are excluded.

It may be that the pharmaceutical composition comprises compound A or compound B.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for sublingual use, for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight may be suitable. Similarly, for administration by inhalation, a dose in the range, for example, 0.05 mg/kg to 25 mg/kg body weight may be suitable. When administered orally a total daily dose of a compound of the invention may be, for example, selected from: 1 mg to 1000 mg, 5 mg to 1000 mg, 10 mg to 750 mg or 25 mg to 500 mg. Typically, unit dosage forms will contain about 0.5 mg to 0.5 g of a compound of the invention. In a particular embodiment the compound of the invention is administered parenterally, for example by intravenous administration. In another particular embodiment the compound of the invention is administered orally.

### Therapeutic Uses and Applications

In this section describing therapeutic uses, applications and methods of treatment reference to “a compound of the invention” includes compounds according to any to any of formulae (I) to (XXXVIc), or a pharmaceutically acceptable salt thereof, except that the compounds A and B 1 are not excluded. Thus “a compound of the invention” in this section may be a compound according to any to any of formulae (I) to (XXXVIc), Compound A and Compound B, or a pharmaceutically acceptable salt thereof.

However, it is to be understood that in some embodiments in this section the compound of the invention may be a compound according to any to any of formulae (I) to (XXXVIc), or a pharmaceutically acceptable salt thereof, with the proviso that the compounds A and B are excluded. In other embodiments in this section the compound of the invention may be a compound selected from Compound A and Compound B, or a pharmaceutically acceptable salt thereof.

In accordance with another aspect, the present invention provides a compound of the invention, for use as a medicament.

A further aspect of the invention provides a compound of the invention, or a pharmaceutically acceptable salt thereof, for use in the prevention or treatment of a disease or medical disorder mediated by Cav2.3.

Also provided is a method of preventing or treating a disease or medical disorder mediated by Cav2.3 in a subject, the method comprising administering to the subject an effective amount of a compound of the invention, or a pharmaceutically acceptable salt thereof.

Also provided is the use of a compound of the invention, or a pharmaceutically acceptable salt thereof, for the manufacture of a medicament for the prevention or treatment of a disease or medical disorder mediated by Cav2.3.

In the following sections of the application reference is made to a compound of the invention, or a pharmaceutically acceptable salt thereof for use in the treatment of certain diseases or medical disorders. It is to be understood that any reference herein to a compound for a particular use is also intended to be a reference to (i) the use of the compound of the invention, or pharmaceutically acceptable salt thereof, in the manufacture of a medicament for the treatment of that disease or disorder; and (ii) a method for the treatment of the disease or disorder in a subject, the method comprising administering to the subject a therapeutically effective amount of the compound of the invention, or pharmaceutically acceptable salt thereof.

In certain embodiments the disease or medical disorder mediated by Cav2.3 is selected from: a neurodegenerative disease, a neurodevelopmental disorder, epilepsy, an endocrine disorder, cerebral vasospasm, and pain.

In certain embodiments there is provided a compound of the invention, or a pharmaceutically acceptable salt thereof, for use in the treatment of a disease or medical disorder selected from: a neurodegenerative disease, a neurodevelopmental disorder, epilepsy, an endocrine disorder, cerebral vasospasm, and pain.

### Neurodegenerative Diseases

In some embodiments the disease or medical disorder is a neurodegenerative disease. For example the disease or medical disorder is selected from: Parkinson's disease, Alzheimer's disease, Huntington's disease, dystonia, amyotrophic lateral sclerosis (ALS), multiple sclerosis, and age-related neurodegeneration. In a particular embodiment a compound of the invention is for use in the treatment of Parkinson's disease.

It may be that a compound of the invention provides a neuroprotective effect in subjects with a neurodegenerative disease. Accordingly, a compound of the invention may be for use in the neuroprotective treatment of a neurodegenerative disease (e.g. Parkinson's disease). In some embodiments a compound of the invention may be for use in preventing or delaying the onset of symptoms associated with a neurodegenerative disease. Thus compound of the invention may be for use in preventing or reducing neurodegeneration associated with a neurodegenerative disease.

In certain embodiments a compound of the invention is for use in preventing or inhibiting degeneration of dopaminergic neurons in a subject with a neurodegenerative disease (e.g. Parkinson's disease). Accordingly, it may be that a compound of the invention is for use in the prevention or inhibition of degeneration of dopaminergic substantia nigra (SN) neurones in a subject with Parkinson's disease.

In certain embodiments a compound of the invention is for use in the treatment or prevention of one or more symptoms of a neurodegenerative disease. For example, a compound may be for use in the treatment or prevention of one or more symptoms of Parkinson's disease selected from: tremor, bradykinesia, dystonia, stiffness, balance, coordination, cognitive impairment, and speech impairment.

### Neurodevelopmental Disorders

In certain embodiments a compound of the invention is for use in the treatment of a neurodevelopmental disorder. In certain embodiments the neurodevelopmental disorder is selected from: CACNA1E Gain-of-function Syndrome (DEE69), CDKL5 Deficiency (DEE2), Fragile X syndrome, Down syndrome, Rett syndrome, Angelman syndrome, autism, motor disorders (e.g., developmental coordination disorder, stereotypic movement disorder and tic disorders), and attention deficit hyperactivity disorder (ADHD).

As discussed in the introduction, Cav2.3 channels are associated with developmental and epileptic encephalopathies (DEEs). The term “DEE” refers to a group a heterogeneous group of rare neurodevelopmental disorders, characterised by (a) early-onset seizures that are often intractable, (b) electroencephalographic abnormalities, (c) developmental delay or regression and (d) in some cases, early death. DEE is classified by the 2017 International League Against Epilepsy (ILAE) Classification of the Epilepsies as an epilepsy associated with developmental impairment that may be due to both the underlying etiology (developmental encephalopathy) and superimposed epileptic activity (epileptic encephalopathy) (Scheffer et al. ILAE classification of the epilepsies: position paper of the ILAE commission for classification and terminology. Epilepsia. 2017; 58:512-21).

In certain embodiments a compound of the invention is for use in the prevention or treatment of a developmental and epileptic encephalopathy. In certain embodiments a compound of the invention is for use in the prevention or treatment of a monogenic developmental and epileptic encephalopathy In certain embodiments a compound of the invention is for use in the treatment or prevention of CACNA1E Gain-of-function Syndrome (DEE69), CDKL5 Deficiency (DEE2), DEE9 (caused by mutation in the PCDH19 gene), DEE11 (SCN2A gain of function), DEE13 (SCN8A gain of function), Dravet syndrome (DEE6A) or a DEE caused by or associated with a loss of function of GABAa receptors (e.g. DEE19, DEE43, DEE45, DEE59, DEE74, DEE78, DEE79 or DEE92).

In one embodiment a compound of the invention is for use in the treatment or prevention of DEE is Dravet syndrome (DEE6A). In a particular embodiment a compound of the invention is for use in the treatment or prevention of CACNA1E Gain-of-function Syndrome (DEE69) or CDKL5 Deficiency (DEE2).

In another embodiment a compound of the invention is for use in the treatment of a DEE caused by or associated with a loss of function of GABAa receptors. For example a compound of the invention is for use in the treatment of a DEE selected from: DEE19, DEE43, DEE45, DEE59, DEE74, DEE78, DEE79 and DEE92.

The genetic phenotypes and clinical features of the DEEs described herein are set out in entry #30008 in the Online Mendelian Inheritance in Man® (OMIM) database (https://www.omim.org/about).

### Epilepsy

In certain embodiments a compound of the invention is for use in the treatment of epilepsy.

Epilepsy is a chronic brain disease in which unprovoked epileptic seizures are the predominant feature. Epileptic seizures can vary from brief and nearly undetectable to long periods of vigorous shaking. Epilepsy and its related syndromes may be classified according to whether seizures are partial or generalized, and whether the aetiology is idiopathic or symptomatic or cryptogenic. The term “epilepsy” comprises both generalized and focal forms, with generalized epilepsy affecting both hemispheres while focal epilepsy includes unifocal and multifocal disorders as well as seizures involving one hemisphere.

In certain embodiments a compound of the invention is for use in the treatment of an epilepsy selected from: idiopathic epilepsy, cryptogenic epilepsy and symptomatic epilepsy. Idiopathic epilepsy is epilepsy with no apparent cause. Cryptogenic epilepsy occurs when the cause of epilepsy in a subject has not been identified despite investigation. Symptomatic epilepsy is epilepsy with a known cause. Causes of symptomatic epilepsy include, for example, brain injury, an bacterial or viral infection (e.g. meningitis), stroke or a tumour.

In some embodiments a compound of the invention is for use in the treatment of an epilepsy syndrome. For example a compound of the invention may be for use in the treatment of an epilepsy syndrome selected from: childhood absence epilepsy, benign Rolandic epilepsy, Doose syndrome, Dravet syndrome, early myoclonic encephalopathy, epilepsy in infancy with migrating focal seizures, Jeavons syndrome, epilepsy with myoclonic absences, epilepsy with generalised tonic-clonic seizures, epileptic encephalopathy with continuous spike and wave during sleep, febrile illness-related epilepsy syndrome, genetic epilepsy with febrile seizures plus, West syndrome, juvenile absence epilepsy, juvenile myoclonic epilepsy, Landau-Kleffner syndrome, Lennox-Gastaut syndrome, myoclonic epilepsy of infancy, Ohtahara syndrome, Panayiotopoulos syndrome, progressive myoclonic epilepsies, reflex epilepsies, self-limited familial and non-familial neonatal-infantile seizures, Gastaut syndrome, sleep-related hypermotor epilepsy, and temporal lobe epilepsy.

In some embodiments a compound of the invention is for use in the treatment or prevention of drug-resistant epilepsy. Drug-resistant epilepsy (also known as “uncontrolled,” “intractable” or “refractory” epilepsy) refers to epilepsy that fails to respond to, or relapses following treatment with an anti-epileptic therapy. Accordingly, in subjects with drug-resistant epilepsy seizures persist despite treatment with one or more anti-epileptic therapies. For example, a subject may not respond to, or relapses after treatment with one or more anti-epileptic therapy (for example the subject does not respond to, or relapses after treatment with at least two anti-epileptic therapies). In certain embodiments the subject fails to respond to, or relapses after treatment with one or more anti-epileptic drug (AED), for example one or more of the AEDs listed herein in relation to combination therapies. The drug-resistant epilepsy may be any of the forms of epilepsy described herein that is, or has become resistant to treatment with one or more (e.g. at least two) anti-epileptic therapy. In some embodiments the drug-resistant epilepsy is a drug-resistant focal epilepsy.

In certain embodiments a compound of the invention is for use in preventing or treating seizures. Thus in certain embodiments a compound of the invention is for use in preventing or treating an epileptic seizure. For example, a compound of the invention may reduce the occurrence of epileptic seizures, reduce the severity and/or duration of epileptic seizures, or reduce the frequency of seizures. In some embodiments a compound of the invention is for use in the prevention or treatment of partial, generalized, convulsive and non-convulsive seizures. In some embodiments a compound of the invention is for use in preventing or treating a seizure selected from: tonic-clonic, tonic, clonic, myoclonic, absence, and atonic seizures.

### Endocrine Disorders

In certain embodiments a compound of the invention is for use in the treatment of an endocrine disorder. For example, a compound of the invention may be for use in the treatment of an endocrine disorder selected from: diabetes (e.g., treating glucose-induced insulin release, glucose-mediated glucagon suppression, or glucose-mediated somatostatin-release), acromegaly, Addison's disease, Cushing's syndrome, Graves' disease, Hashimoto's thyroiditis, hyperthyroidism, hypothyroidism (underactive thyroid), and prolactinoma.

### Pain

In certain embodiments a compound of the invention is for use in the treatment or prevention of pain. In some embodiments a compound of the invention is for use in the treatment of chronic pain, inflammatory pain, neuropathic pain (e.g. peripheral neuropathic pain or central neuropathic pain), or nociceptive pain.

### Cerebral Vasospasm

Subjects which suffer a cerebral aneurism or aneurysmal subarachnoid haemorrhage (bleeding on the surface of the brain) often survive the initial trauma. However, often within a few days to two weeks subjects experience cerebral vasospasm, a constriction, or tightening, of arteries in the brain. Cerebral vasospasm restricts blood flow to the brain and may subsequently lead to the death of blood-starved brain tissue resulting in cerebral infarction. Expression of Cav2.3 may be increased following a cerebral aneurism or aneurysmal subarachnoid haemorrhage and may be implicated in cerebral vasospasm (Wang et al., supra). Accordingly, in some embodiments a compound of the invention is for use in the treatment or prevention of cerebral vasospasm. For example a compound of the invention is for use in the treatment or prevention of cerebral vasospasm in a subject who has suffered a cerebral aneurism or aneurysmal subarachnoid haemorrhage. In some embodiments a compound of the invention is for use in the treatment or prevention of cerebral infarction.

### Selectivity

Without wishing to be bound by theory it is expected that the selective modulation of Cav2.3 will provide compounds with a desirable therapeutic effect whilst avoiding or minimising the side effects associated with a non-selective Cav2.3 antagonist.

In certain embodiments such selective compounds may be used in the treatment or prevention of any of the diseases or medical disorders described herein.

### Combination Therapies

The compounds of the invention may be used alone to provide a therapeutic effect. The compounds of the invention may also be used in combination with one or more additional therapeutic agents.

In some embodiments the additional therapeutic agent is selected from one or more of:
an anti-epileptic drug (AED), for example acetazolamide, benzodiazepine, cannabadiols, carbamazepine, clobazam, clonazepam, diazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, valproic acid, sodium valproate, soticlestat, stiripentol, tiagabine, topiramate, vigabatrin, or zonisamide.
a drug for the treatment of Parkinson's disease, for example a dopamine mimetic (substances which regulate/modulate the dopamine metabolism, e.g., levodopa or carbidopa); a dopamine receptor agonist (e.g. pramipexole, ropinirole, rotigotine or apomorphine); a monaminoxidase inhibitor, for example an MAO B inhibitor (e.g. selegiline, rasagiline or safinamide); a catechol O-methyltransferase (COMT) inhibitor (e.g. entacapone, opicapone or tolcapone); an anticholinergic (e.g. benztropine or trihexyphenidyl); adamantane; or an adenosine A2A receptor antagonist (e.g. istradefylline).

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

### Biological Assays

The effect of a compound of the invention on inhibiting calcium ion influx into cells via human Cav2.3 channels can be assessed using the human Cav2.3 channel calcium-influx assay described in the examples section. The effects of compounds of the invention inhibiting the function of Cav2.3 ion channels in-vitro can be assessed by, for example using whole cell patch clamp methods such as that described in the example section.

The effects of the compounds blocking R-type calcium current with whole cell patch clamp electrophysiology in substantia nigra dopamine neurons in an ex vivo brain slice can be assessed using the methods described in Siller et al., Elife, 11: e67464 (2022) https://doi.org/10.7554/eLife.67464.

Effects of the compounds on diseases or medical disorders mediated by Cav2.3 may be assessed using suitable in-vitro and in-vivo models for such diseases and medical disorders. For example, the effects of a compound of the invention on Parkinson's disease may be assessed using the methods and models described in WO2018/228692. Other suitable models for Parkinson's disease include, for example, the MitoPark mouse model described in Galter et al. (Genes Brain Behav. 2010 Mar. 1; 9 (2): 173-181); and the SNCA-OVX transgenic mouse model described in Janezic et al. (Proceedings of the National Academy of Sciences, 2013 September, 201309143 DOI: 10.1073/pnas. 1309143110).

Suitable models for testing a compound of the invention for the treatment of seizures or epilepsy include, for example, one or more of the models described in Löscher (Seizure, 2011, (20), 359-368). Alternatively a compound of the invention may be tested in the Maximal Electroshock Stimulation (MES) model described in Kehne et al, Neurochemistry Research 42:1894-1903 (2017); https://doi.org/10.1007/s11064-017-2275-z.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

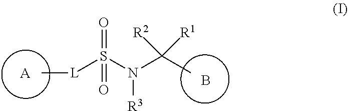

Compounds of formula (I) can generally be prepared by reacting a compound of formula (Ia′)

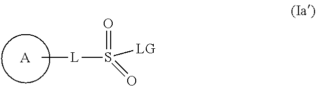

wherein ring A and L are as defined above for any of formulae (I) to (XXXVIc), and LG is a leaving group, with a compound of formula (Ib) or with a compound of formula (Ic):

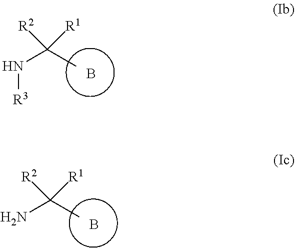

wherein R¹, R², R³, and ring B are as defined above for any of formulae (I) to (XXXVIc), except that any functional group is protected, if necessary;
and optionally thereafter carrying out one or more of the following procedures:
converting a compound of formula (I) into another compound of formula (I); removing any protecting groups; forming a pharmaceutically acceptable salt; and/or preparing stereochemically isomeric forms thereof.

Compounds of formula (I) may also be prepared by reacting a compound of formula (Id):

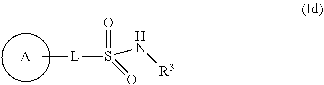

wherein ring A, L, and R³ are as defined above for any of formulae (I) to (XXXVIc), with a compound of formula (Ie)

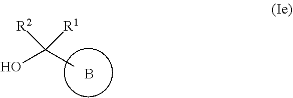

wherein ring B, R¹, and R² are as defined above for any of formulae (I) to (XXXVIc), except that any functional group is protected, if necessary;
and optionally thereafter carrying out one or more of the following procedures:
converting a compound of formula (I) into another compound of formula (I); removing any protecting groups; forming a pharmaceutically acceptable salt; and/or preparing stereochemically isomeric forms thereof.

The reaction of a compound of formula (Ia′) with a compound of formula (Ib/Ic), may be carried out in a reaction-inert solvent for example, DCM, THF, acetonitrile, and optionally in the presence of at least one suitable base thereof. Non-limiting examples of such reaction promoters include DIPEA, TEA, pyridine, NMM, 2,6-lutidine, DMAP or a functional derivative thereof.

In the compound of formula (Ia) LG is an appropriate leaving group such as, for example, halo, e.g., fluoro, chloro, bromo and the like.

The reaction of a compound of formula (Ia′) with a compound of formula (Ib), may be performed in a reaction-inert solvent such as, for example, DCM, THF, acetonitrile, pyridine, and optionally in the presence of a suitable base such as, for example, sodium carbonate, potassium carbonate or trimethylamine, DIPEA, pyridine. Stirring may enhance the rate of the reaction. The reaction may conveniently be carried out at a temperature ranging between water freezing temperature (0° C.) and the reflux temperature of the reaction mixture.

Compounds of formula (Ia′) may be prepared according to the following scheme (General Scheme 1):

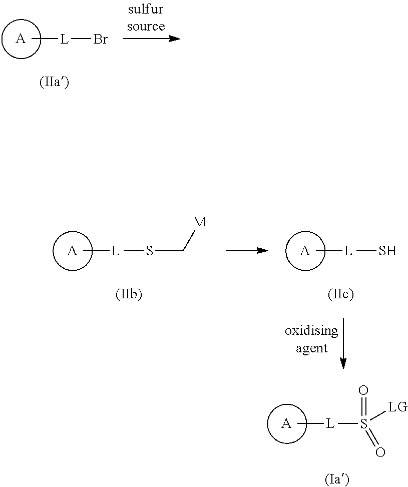

Sulfide compound (IIb) may be obtained by reaction of the respective bromo derivatives (IIa′) in a sulfur-carbon bond forming reaction in an inert atmosphere in the presence of Pd catalyst. Examples of such reaction include reaction with aromatic bromo, chloro or iodo compound react with sulphur compound e.g. phenylmethanethiol, (4-methoxyphenyl) methanethiol and 2-ethylhexyl 3-mercaptopropanoate. The reaction may be performed in a suitable solvent, such as, 1,4-dioxane, toluene, benzene, DMF, DME, DMA solution preferably at temperatures between rt 150° C. Palladium-catalyzed coupling reactions, the active palladium catalyst is believed to be Pd (0) complex, which can be generated in a variety of ways. Suitable Pd(0) sources such as, Pd(PPh₃)₄ or Pd(dba)₂, can undergo ligand dissociation to form the active species. Phosphines can be added to ligandless palladium(0).

Sulfide compound (IIc) can be obtained by the reaction of the respective sulfide derivatives (IIb) in a sulfur-carbon bond cleavage reaction in an inert atmosphere in the presence of a base. Suitable bases for this type of conversion used include, for example, NaOEt, NaOMe, K₂CO₃, Na₂CO₃, tBuONa and tBuOK. Suitable solvents for this type of conversion include, for example, EtOH, MeOH, THF, DCE, DCM, MeCN preferably at temperatures between −78° C. and RT.

Sulfonyl chloride (Ia′) may be obtained by reaction of the respective thiol derivatives (IIc) in a sulfur-oxygen and sulfur-chlorine bond forming reaction. Non-limiting examples of such reaction include reaction with:
halogen or a chlorine source in the presence of acid water such as chlorine gas, NaOCl, NCS, NBS, 1,3-Dibromo-5,5-Dimethylhydantoin, oxone, isocyanuric chloride or a derivative thereof;
an oxygen source such as ammonium nitrate, an aqueous solution of AcOH, HCl and HBr and oxygen as a terminal oxidant was developed in that process.

The reaction may be performed in a suitable solvent, such as, DCM, tetrahydrofuran, acetic acid, diethyl ether, toluene preferably at temperatures between −20° C. and RT.

Compounds of formula (Id), wherein the meanings of ring A, R³, L are as defined above for any of formulae (I) to (XXXVIc), and LG is a leaving group, can be prepared via a single step procedure from formula (II) and can be converted to the title compound of formula (I) using Mitsunobu reaction as mentioned in the following scheme (General Scheme 2):

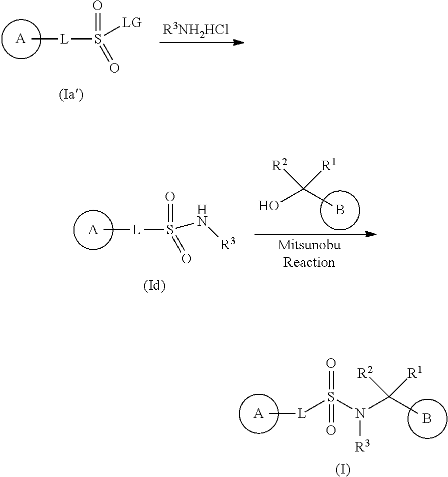

Reaction of compounds of formula (Ia′) with an amine or it's salt, with or without base, under thermal condition, preferably at temperatures between 0° C. and room temperature. The reaction may be carried out in a reaction-inert solvent and optionally in the presence of at least one suitable base thereof. Non-limiting examples of such reaction promoters include DIPEA, TEA, pyridine, NMM, 2,6-lutidine, DMAP or a functional derivatives thereof.

LG in the compound of formula (Ia′) is an appropriate leaving group such as, for example, halo, e.g., fluoro, chloro, bromo and the like.

Reaction of compounds of formula (Id) with a substituted benzyl alcohol reagent of the type of formula (Ie):

Phosphine source of this type of conversion generally used are triphenylphosphine, tricyclohexylphosphine, tributylphosphine, trihexylphosphine or a functional derivative thereof. Azo compound source of this type of reaction generally used are DEAD, DIAD, DTAB, ADDP or a functional derivative thereof. The conversion may preferably be performed in a reaction-inert solvent such as, for example, dioxane, THF, diethyl ether.

Compounds of formula (If) may generally be prepared by reacting a compound of formula (Ia′) and formula (Ic), using the protocol described above. Compounds according to formula (I) can be prepared from a single step procedure form formula (If) using N-alkylation as mentioned below the following scheme (General Scheme 3):

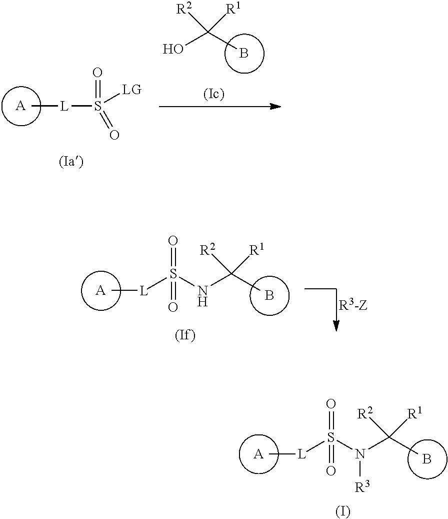

where Z in the alkylating compound (R³—Z) is an appropriate leaving group, such as, for example, halo, e.g., fluoro, chloro, bromo, iodo and the like.

Reaction of compounds of formula (If) with alkylating reagent, preferably with inorganic base under thermal condition preferably at temperatures between 50° C. and 120° C. The reaction of a compound of formula (If), may be carried out in a at least one reaction-inert solvent and optionally in the presence of at least one suitable base thereof. Non-limiting examples of such reaction promoters include K₂CO₃, Cs₂CO₃, NaH or a functional derivative thereof; may be performed in a reaction-inert solvent such as, for example, DMF, DMSO, acetonitrile.

Certain of the intermediates described herein, for example Intermediate (If), and salts thereof, form a further aspect of the invention. In the intermediate of formula (If) Ring A, Ring B, R¹, R² and L are as defined for formula (I), including any of the values in numbered paragraphs 1 to 223 above. In certain embodiments the intermediate of formula (If) is a compound of formula (If) as described in any of the Examples herein, or a salt thereof.

Some of the compounds of the formula (If) have activity as Cav2.3 antagonists. Accordingly, in another embodiment there is provided a compound of any of formulae (I) to (XXXVIc), or a pharmaceutically acceptable salt thereof, wherein R³ is selected from: H, C₁₋₆ alkyl and C₁₋₆ haloalkyl. Suitably in this embodiment R³ is H. In these embodiments Ring A, Ring B, R¹, R² and L are as defined for formula (I), including any of the values in numbered paragraphs 1 to 223 above. Thus the compound may be a compound of the formula (If) above, for example, one of the compounds of formula (If) used in the preparation of any of the Examples herein.

### Further Embodiments

The invention is further illustrates be the following embodiments.
P1 A compound of the formula (I), or a pharmaceutically acceptable salt thereof:

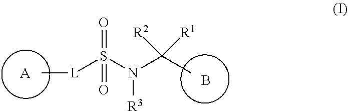

wherein:
R¹ is selected from: C₁₋₆ alkyl, C₃₋₆ cycloalkyl, and C₃₋₆ cycloalkyl-C₁₋₆ alkyl-, wherein R¹ is substituted by at least one fluorine;
R² is selected from: H, C₁₋₆ alkyl and C₁₋₆ haloalkyl; or
R¹ and R² together with the carbon atom to which they are attached form a C₃₋₆ cycloalkyl substituted with at least one fluorine;
R³ is selected from: C₁₋₆ alkyl and C₁₋₆ haloalkyl;
L is selected from: a bond and C₁₋₃ alkylene;
Ring A is selected from: C₃₋₆ cycloalkyl, 4- to 7-membered heterocyclyl, 5- to 12-membered heteroaryl and C₆₋₁₀ aryl; wherein Ring A is optionally substituted by one or more R⁴;
each R⁴ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷;
R⁵ and R⁶ are each independently selected from: H, C₁₋₆ alkyl, C₁₋₆ haloalkyl and Q¹,
   wherein said C₁₋₆ alkyl is optionally substituted by one or more R⁸;
each R⁷ and R⁸ is independently selected from: halo, —CN, —OR^{7A}, —S(O)ₓR^{7A}, —NR^{7A}R^{7B}, C(O)R^{7A}, —OC(O)R^{7A}, —C(O)OR^{7A}, —NR^{7A}C(O)R^{7B}, —C(O)NR^{7A}R^{7B} and Q²;
each Q¹ and Q² is independently selected from: C₃₋₆ cycloalkyl, 4- to 7-membered heterocyclyl, phenyl and 5- or 6-membered heteroaryl,
   wherein said C₃₋₆ cycloalkyl, 4- to 7-membered heterocyclyl, phenyl and 5- or 6-membered heteroaryl is optionally substituted by one or more R⁹;
   each R⁹ is independently selected from: halo, ═O, —CN, —NO₂, C₁₋₄ alkyl, C₁₋₄ haloalkyl, —OR^{9A}, —S(O)₂R^{9A}, —NR^{9A}R^{9B}, —C(O)R^{9A}, —OC(O)R^{9A}, —C(O)OR^{9A}, —NR^{9B}C(O)R^{9A}, —C(O)NR^{9A}R^{9B}, —NR^{9B}C(O)OR^{9A}, —OC(O)NR^{9A}R^{9B}, —NR^{9B}SO₂R^{9A} and —SO₂NR^{9A}R^{9B},
   wherein said C₁₋₄ alkyl is optionally substituted by 1 or 2 substituents selected from: halo, —CN, —OR^{9C}, —NR^{9C}R^{9D} and —SO₂R^{9C};
Ring B is phenyl or a 5- or 6-membered heteroaryl, wherein Ring B is optionally substituted by one or more R¹⁰;
   each R¹⁰ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
   wherein said C₁₋₆ alkyl, 2 to 8 membered heteroalkyl, C₂₋₆ alkenyl and C₂₋₆ alkynyl is optionally substituted by one or more R¹¹;
   each R¹¹ is independently selected from: halo, —CN, —OR^{11A}, —NR^{11A}R^{11B} and —SO₂R^{11A};
R^{7A}, R^{7B}, R^{9A}, R^{9B}, R^{9C}, R^{9D}, R^{10A}, R^{10B}, R^{11A} and R^{11B} are at each occurrence independently selected from: H, C₁₋₄ alkyl and C₁₋₄ haloalkyl;
   and wherein any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
each x is independently 0, 1, or 2;
with the proviso that Compounds A and B are excluded:

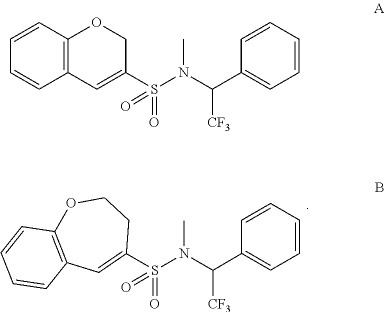 [SMILES: CN(C(c1ccccc1)C(F)(F)F)S(=O)(=O)C1=Cc2ccccc2OC1 ; CN(C(c1ccccc1)C(F)(F)F)S(=O)(=O)C1=Cc2ccccc2OCC1]

P2. The compound according to embodiment 1, wherein Ring A is selected from furanyl, thienyl, imidazolyl, pyrazolyl, oxazolyl, isoxazolyl, thiazolyl, isothiazolyl, thiadiazolyl, phenyl, pyridyl, pyrimidinyl, pyrazinyl, or a compound of the structure:

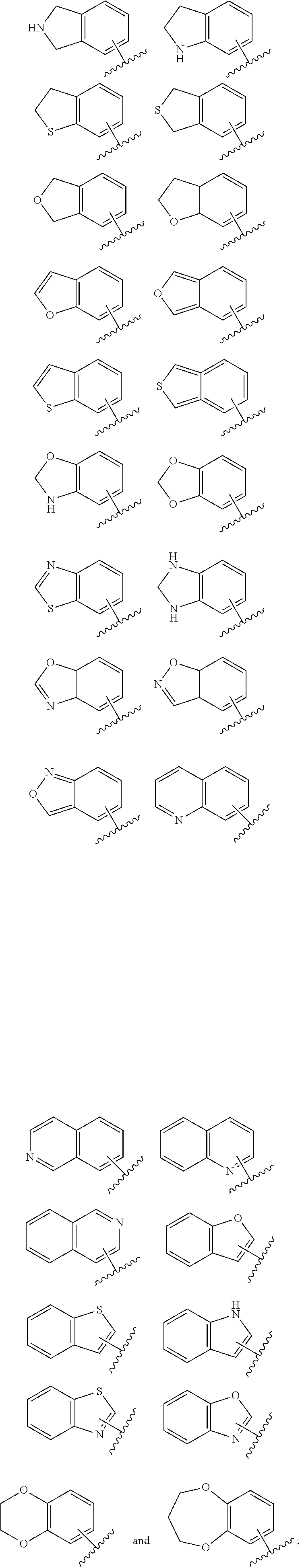

wherein Ring A is optionally substituted with one or more R⁴.
P3. The compound according to embodiment 1 or embodiment 2, wherein each R⁴ is independently selected from: halo, —CN, —NO₂, ═O, C₁₋₈ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵SO₂R⁶, and —SO₂NR⁵R⁶.
P4. The compound according to embodiment 1, wherein the compound is a compound of the formula (III), or a pharmaceutically acceptable salt thereof:

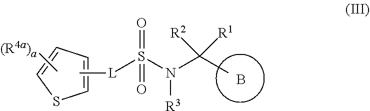

wherein:
each R^{4a} is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, C₁₋₆ heteroalkyl, C₂₋₆ alkenyl and C₂₋₆ alkynyl is optionally substituted by one or more R⁷; and
a is an integer from 0 to 3.
P5. The compound according to embodiment 1, wherein the compound is a compound of the formula (VII), or a pharmaceutically acceptable salt thereof:

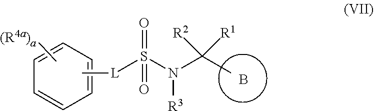

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and
a is an integer from 0 to 5.
P6. The compound according to embodiment 1, wherein the compound is a compound of the formula (XI), or a pharmaceutically acceptable salt thereof:

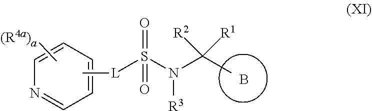

wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
   wherein said C₁₋₆ alkyl, 2 to 8 membered heteroalkyl, C₂₋₆ alkenyl and C₂₋₆ alkynyl is optionally substituted by one or more R⁷; and a is an integer from 0 to 4.
P7. The compound according to any one of embodiments 1 to 6, wherein L is selected from a bond and —CH₂—.
P8. The compound according to any one of embodiments 1 to 6, wherein L is a bond.
P9. The compound according to any one of embodiments 1 to 8, wherein R¹ is selected from C₁₋₆ alkyl and C₃₋₆ cycloalkyl, wherein R¹ is substituted by at least one fluorine.
P10. The compound according to any one of embodiments 1 to 8, wherein R¹ is selected from —CH₂F, —CHF₂, and —CF₃.
P11. The compound according to any one of embodiments 1 to 8, wherein R¹ is —CF₃.
P12. The compound according to any one of embodiments 1 to 8, wherein R¹ and R², together with the carbon atom to which they are attached, form a C₃ or C₄ cycloalkyl substituted with at least one fluorine.
P13. The compound according to any one of embodiments 1 to 12, wherein R² is selected from H and methyl.
P14. The compound according to any one of embodiments 1 to 12, wherein R² is H.
P15. The compound according to any one of embodiments 1 to 14, wherein R³ is selected from: C₁₋₃ alkyl; optionally substituted with one or more halo groups.
P16. The compound according to any one of embodiments 1 to 15, wherein Ring B is selected from furanyl, thienyl furanyl, thienyl, imidazolyl, pyrazolyl, oxazolyl, isoxazolyl, thiazolyl, isothiazolyl, thiadiazolyl, phenyl, pyrimidinyl, and pyrazinyl; wherein Ring B is optionally substituted by one or more R¹⁰.
P17. The compound according to any one of embodiments 1 to 15, wherein Ring B is phenyl optionally substituted by one or more R¹⁰.
P18. The compound according to any one of embodiments 1 to 17, wherein each R¹⁰ is independently selected from: halo, —CN, —NO₂, C₁₋₆ alkyl, C₁₋₆ haloalkyl, —OR^{10A}, —S(O)ₓR^{10A} and —NR^{10A}R^{10B}.
P19. The compound according to any one of embodiments 1 to 17, wherein each R¹⁰ is independently selected from: halo and C₁₋₄ alkyl.
P20. The compound according to any one of embodiments 1 to 15, wherein Ring B is phenyl substituted by one or two R¹⁰ selected from halo and C₁₋₄ alkyl, for example wherein Ring B is 4-fluorophenyl.
P21. A compound selected from Compound List 1 in the description, or a pharmaceutically acceptable salt thereof.
P22. A pharmaceutical composition comprising a compound according to any one of embodiments 1 to 21, except that Compounds A and B are not excluded, or a pharmaceutically acceptable salt thereof, and a pharmaceutically acceptable excipient.
P23. A compound according to any one of embodiments 1 to 21, except that Compounds A and B are not excluded, or a pharmaceutically acceptable salt thereof, for use as a medicament.
P24. A compound according to any one of embodiments 1 to 21, except that Compounds A and B are not excluded, or a pharmaceutically acceptable salt thereof, for use in the treatment of a disease or medical disorder mediated by Cav2.3.
P25. A method of treating a disease or medical disorder mediated by Cav2.3 in a subject in need thereof, the method comprising administering to the subject an effective amount of a compound according to any one of embodiments 1 to 21, except that Compounds A and B are not excluded, or a pharmaceutically acceptable salt thereof.
P26. A compound according to any one of embodiments 1 to 21, except that Compounds A and B are not excluded, or a pharmaceutically acceptable salt thereof, for use in in the treatment of a disease or medical disorder selected from: a neurodegenerative disease, a neurodevelopmental disorder, epilepsy, an endocrine disorder, cerebral vasospasm, and pain.
P27. A compound according to any one of embodiments 1 to 21, except that Compounds A and B are not excluded, or a pharmaceutically acceptable salt thereof, for use in a neuroprotective treatment of a neurodegenerative disease.
P28. A compound according to any one of embodiments 1 to 21, except that Compounds A and B are not excluded, or a pharmaceutically acceptable salt thereof, for use in the treatment of Parkinson's disease.
P29. A compound according to any one of embodiments 1 to 21, except that Compounds A and B are not excluded, or a pharmaceutically acceptable salt thereof, for use in a preventing or inhibiting degeneration of dopaminergic neurons in a subject with Parkinson's disease.

### EXAMPLES

### Abbreviations

Ac—acetyl
BINAP—2,2′-bis(diphenylphosphino)-1,1′-binaphthyl
Bn—benzyl
Boc—tert-butoxycarbonyl
CBz—benzyloxycarbonyl
CPME—cyclopentyl methyl ether
dba—dibenzylideneacetone
DCM—dichloromethane
DIEA—N,N-diisopropylethylamine
DIPA—diisopropylamine
DMAc—dimethylacetamide
DMF—N,N-dimethylformamide
DMSO—dimethylsulfoxide
EDCI—1-ethyl-3-(3-dimethylaminopropyl) carbodiimide hydrochloride salt
ee—enantiomeric excess
eq.—equivalents
Ghosez's Reagent—1-chloro-N,N-2-trimethyl-1-propenylamine
HATU—1-[bis(dimethylamino)methylene]-1H-1,2,3-triazolo[4,5-b]pyridinium 3-oxide hexafluorophosphate
HOAt—1-hydroxy-7-azabenzotriazole
HPLC—high performance liquid chromatography
IPA—isopropanol
KHMDS—potassium bis(trimethylsilyl)amide
LC-MS—liquid chromatograph-mass spectrometer
LDA—lithium diisopropylamide
mCPBA—3-chloroperbenzoic acid
MeCN—acetonitrile
MS—mass spectrometry
Ms—mesyl
MTBE—methyl tert-butyl ether
MW—microwave
NBS—N-bromosuccinimide
NMM—N-methylmorpholine
NMP—N-methyl-2-pyrrolidone
NMR—nuclear magnetic resonance
o/n—overnight
Pd/C—palladium-on-carbon
Piv—pivaloyl
Prep—preparative
pTSA—p-toluene sulfonic acid
Py—pyridine
rt—retention time
RT—room temperature
RM—reaction mass
SFC—supercritical fluid chromatography
SEM—trimethylsilylethoxymethyl
SPE—solid phase extraction
Su—succinimide
TBAB—tetrabutylammonium bromide
TBAF—tetrabutylammonium fluoride
TEA—triethylamine
TFA—trifluoroacetic acid
TFAA—trifluoroacetic anhydride
THF—tetrahydrofuran
TLC—Thin-layer chromatography

### Reagents and Conditions

Unless syntheses are given, reagents and starting materials were obtained from commercial sources. All reactions, unless otherwise stated, were carried out under an inert atmosphere of either nitrogen or argon.

### Compound Names

New compounds were named using ChemDraw Ultra 14.0 from CambridgeSoft. Other compounds, particularly commercial reagents, either use names generated by ChemDraw Ultra 14.0 or names commonly found in online databases and catalogues.

### Analytical Methods

### TCGLS LCMS Method

### Method 1 (K84-3 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/min. Two mobile phases were used, mobile phase A: 0.05% HCOOH in water; mobile phase B: 0.05% HCOOH in ACN:Water (90:10)], and they were employed to run a gradient conditions from 10% B for 0.75 minutes, from 10% to 50% in 0.25 minutes, and from 50% to 98% in 1.00 minutes, 98% B for 0.25 minutes and then 10% B in 0.35 minutes and hold these conditions for 0.40 minutes in order to re-equilibrate the column (Total Run Time 3.00 minutes). An injection volume of 0.5 μl was used.

### Method 2 (K84/K92-5 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/min. Two mobile phases were used, mobile phase A: 0.05% HCOOH in water; mobile phase B: 0.05% HCOOH in ACN:Water (90:10)], and they were employed to run a gradient conditions from 5% B for 0.75 minutes, from 5% to 25% in 0.75 minutes, and from 25% to 95% in 1.50 minutes, 95% B for 1.00 minutes and 5% B in 0.50 minutes and hold these conditions for 0.60 minutes in order to re-equilibrate the column (Total Run Time 5.10 minutes). An injection volume of 0.5 μl was used.

### Method 3 (K70/71/55/63 3 Min)

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 to 3 μl was used (Depending on the sample concentration).

### Method 4 (K03/04/05/06/07/08/72/78 5 Min)

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax C18 column (5 μm, 50×4.6 mm) with a flow rate of 1.20 ml/min. Two mobile phases were used, mobile phase A: 10 mm Ammonium Acetate in water; mobile phase B: ACN, and they were employed to run a gradient condition from 10% B to 30% B in 1.50 minutes, and from 30% to 90% in 1.50 minutes, 90% B for 1.00 minutes and 10% B in 1.00 minutes and hold these conditions for 0.10 minutes. Pre run Equilibration Time 0.50 min (Total Run Time 5.10 minutes). An injection volume of 1 μl to 3 μl was used (Depending on the sample concentration).

### Method 5 (K03/04/05/06/07/08/72/78 5 Min Nonpolar)

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax C18 column (5 μm, 50×4.6 mm) with a flow rate of 1.20 ml/min. Two mobile phases were used, mobile phase A: 10 mm Ammonium Acetate in water; mobile phase B: ACN, and they were employed to run a gradient condition from 50% B to 95% B in 1.50 minutes, and 95% B for 2.50 minutes and 50% B in 1.00 minutes and hold these conditions for 0.10 minutes. Pre run Equilibration Time 0.50 min (Total Run Time 5.10 minutes). An injection volume of 1 μl to 3 μl was used (Depending on the sample concentration).

### Method 6 ((K03/04/05/06/07/08/72/78 12 Min)

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax C18 column (5 μm, 50×4.6 mm) with a flow rate of 1.00 ml/min. Two mobile phases were used, mobile phase A: 10 mm Ammonium Acetate in water); mobile phase B: ACN, and they were employed to run a gradient condition from 5% B for 1.00 min, from 5% B to 50% B in 6.00 minutes, and 50% B to 90% B in 3.00 minutes and 90% B for 1.00 minutes and 5% B in 1.00 minutes and hold these conditions for 0.10 minutes. Pre run Equilibration Time 0.50 min (Total Run Time 12.10 minutes). An injection volume of 1 μl to 3 μl was used (Depending on the sample concentration).

### Method 7 (K70/71/55/63 12 Min)

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a Waters YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 5% B for 1.00 minutes, from 5% to 50% in 4.00 minutes, and from 50% to 90% in 3.00 minutes, 90% B for 2.00 minutes and then 5% B in 1.50 minutes and hold these conditions for 0.50 minutes in order to re-equilibrate the column (Total Run Time 12.00 minutes). An injection volume of 0.5 to 3 μl was used (Depending on the sample concentration).

### Method 8 (K83 3 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (3.5 μm, 50×3 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient conditions from 5% B for 0.75 minutes, from 5% to 30% in 0.25 minutes, and from 30% to 98% in 1.00 minutes, 98% B for 0.25 minutes and 5% B in 0.50 minutes and hold these conditions for 0.25 minutes in order to re-equilibrate the column (Total Run Time 3.00 minutes). An injection volume of 0.5 μl was used.

### Method 9 (K83 5 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (3.5 μm, 50×3 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient conditions from 5% B for 0.75 minutes, from 5% to 15% in 0.50 minutes, from 15% to 70% in 1.25 minutes and from 70% to 98% in 1.25 minutes, 98% B for 0.50 minutes and 5% B in 0.25 minutes and hold these conditions for 0.60 minutes in order to re-equilibrate the column (Total Run Time 5.10 minutes). An injection volume of 0.5 μl was used.

### Method 10 (K83 12 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (3.5 μm, 50×3 mm) with a flow rate of 1.00 ml/min. Two mobile phases were used, mobile phase A: 5 Mm NH4oAc in water; mobile phase B: 5 Mm NH4oAc in ACN:Water (90:10)], and they were employed to run a gradient conditions from 2% B for 1.00 minutes, from 2% to 50% in 4.00 minutes and from 50% to 98% in 3.00 minutes, 98% B for 2.00 minutes and 2% B in 2.00 minutes and hold these conditions for 0.10 minutes in order to re-equilibrate the column (Total Run Time 12.10 minutes). An injection volume of 0.5 μl was used.

### Method 11 (K84 12 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/min. Two mobile phases were used, mobile phase A: 0.05% HCOOH in water; mobile phase B: 0.05% HCOOH in ACN:Water (90:10)], and they were employed to run a gradient conditions from 5% B for 1.00 minutes, from 5% to 50% in 4.00 minutes, and from 50% to 90% in 3.00 minutes, 90% B for 2.00 minutes and then 5% B in 1.50 minutes and hold these conditions for 0.50 minutes in order to re-equilibrate the column (Total Run Time 12.00 minutes). An injection volume of 0.5 μl was used.

### Method 12 (K79 3 Min)

The HPLC measurement was performed using Agilent 1260 Infinity II UPLC comprising a quaternary pump with degasser, an sample manager, a column oven (set at 50° C.), a diode-array detector DAD and a column as specified in the respective methods below. Flow from the column was split to a MS spectrometer. The MS detector (Agilent SQD) was configured with an electrospray ionization source. Mass spectra were acquired by scanning from 100 to 1200 in 0.20 second. The capillary needle voltage was 4.00 kV in positive and negative ionization mode and the source temperature was maintained at 350° C. Nitrogen was used as the desolvation gas, the flow was 12 L/Min. Data acquisition was performed with Open Lab CDS. Reversed phase HPLC was carried out on a YMC Triart C18 column (3 μm, 33×2.1 mm) with a flow rate of 1.00 ml/min. Two mobile phases were used, mobile phase A: 0.1% HCOOH in water; mobile phase B: 0.1% HCOOH in ACN, and they were employed to run a gradient condition from 2% B for 0.50 minutes, from 2% to 30% in 0.50 minutes, and from 30% to 98% in 1.00 minutes, 98% B for 0.25 minutes and then 2% B in 0.75 minutes (Total Run Time 3.00 minutes). An injection volume of 0.5 μl was used.

### Method 13 (K83 3 Min-BEH)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 KV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 30×2.1 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient conditions from 2% B for 0.50 minutes, from 2% to 98% in 1.00 minutes, 98% B for 1.00 minutes and 2% B in 0.25 minutes and hold these conditions for 0.25 minutes in order to re-equilibrate the column (Total Run Time 3.00 minutes). An injection volume of 0.5 μl was used.

### Method 14 (K83 12 Min)-Old

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (5 μm, 50×4.6 mm) with a flow rate of 1.50 ml/min. Two mobile phases were used, mobile phase A: 5 Mm NH4oAc in water; mobile phase B: 5 Mm NH4oAc in ACN:Water (90:10)], and they were employed to run a gradient conditions from 2% B for 0.75 minutes, from 2% to 15% in 0.50 minutes, from 15% to 70% in 1.25 minutes and from 70% to 98% in 1.25 minutes, 98% B for 0.75 minutes and 2% B in 0.50 minutes and hold these conditions for 0.10 minutes in order to re-equilibrate the column (Total Run Time 5.10 minutes). An injection volume of 0.5 μl was used.

### Method 15 (K83 12 Min)-Old

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 KV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (5 μm, 50×4.6 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient conditions from 2% B for 1.00 minutes, from 2% to 50% in 4.00 minutes, from 50% to 90% in 3.00 minutes and 90% B for 2.00 minutes and 2% B in 2.00 minutes and hold these conditions for 0.10 minutes in order to re-equilibrate the column (Total Run Time 12.10 minutes). An injection volume of 0.5 μl was used.

### Method 16

The HPLC measurement was performed using Waters Acquity UPLC comprising a quaternary pump with degasser, a sample manager, a column oven (set at 50° C.), a diode-array detector DAD and a column as specified in the respective methods below. Flow from the column was split to a MS spectrometer. The MS detector (Waters ZQ) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Sunfire C18 column (5 μm, 100×4.6 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient condition from 2% B for 1.00 minutes, from 2% to 50% in 4.00 minutes, and from 50% to 95% in 4.00 minutes, 95% B for 3.00 minutes and then 5% B in 0.50 minutes. (Total Run Time 12.50 minutes). An injection volume of 0.5 μl was used.

### Method 17

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax Ext C18 column (5 μm, 100×4.6 mm) with a flow rate of 1.00 ml/min. Two mobile phases were used, mobile phase A: 10 mm Ammonium Acetate in water); mobile phase B: ACN, and they were employed to run a gradient condition from 50% B for 2.00 min, from 50% B to 95% B in 6.00 minutes, and 95% B for 3.00 minutes and 50% B in 3.00 minutes and hold these conditions for 4.00 minutes. Pre run Equilibration Time 4.00 min (Total Run Time 18.00 minutes). An injection volume of 1 μl to 3 μl was used (Depending on the sample concentration).

### Method 18 (K84 3 Min)-Old

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 μl was used.

### Method 19 (K84 3 Min)-Old BEH Polar

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.80 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 μl was used.

### Method 20 (K43 6 Min)

The HPLC measurement was performed using Agilant HPLC comprising a binary pump with degasser, a sample manager a DAD and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax C18 column (5 μm, 50×4.6 mm) with a flow rate of 1.20 ml/min. Two mobile phases were used, mobile phase A: 10 mm Ammonium Acetate in water); mobile phase B: ACN, and they were employed to run a gradient condition from 10% B to 30% B in 1.50 minutes, and from 30% to 90% in 1.50 minutes, 90% B for 1.00 minutes and 10% B in 1.00 minutes and hold these conditions for 1.00 minutes. Pre run Equilibration Time 0.50 min (Total Run Time 6.00 minutes). An injection volume of 1 μl to 3 μl was used (Depending on the sample concentration).

### Method 22 (K91 12 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 50×2.1 mm) with a flow rate of 0.60 ml/min. Two mobile phases were used, mobile phase A: 5 Mm NH4oAc in water; mobile phase B: 5 Mm NH4oAc in ACN:Water (90:10)], and they were employed to run a gradient conditions from 2% B for 1.00 minutes, from 2% to 50% in 4.00 minutes, from 50% to 98% in 3.00 minutes 98% B for 2.00 minutes and 2% B in 1.00 minutes and hold these conditions for 1.00 minutes in order to re-equilibrate the column (Total Run Time 12.00 minutes). An injection volume of 0.5 μl was used.

### Method 23 (K91 3 Min)

The HPLC measurement was performed using Waters Acquity H Class UPLC comprising a quaternary pump with degasser, a sample manager, a column oven (set at 40° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 KV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 50×2.1 mm) with a flow rate of 0.60 ml/min. Two mobile phases were used, mobile phase A: 5 Mm NH4oAc in water; mobile phase B: 5 Mm NH4oAc in ACN:Water (90:10)], and they were employed to run a gradient conditions from 5% B for 0.75 minutes, from 5% to 30% in 0.25 minutes, from 30% to 98% in 1.00 minutes 98% B for 0.25 minutes and 5% B in 0.25 minutes and hold these conditions for 0.50 minutes in order to re-equilibrate the column (Total Run Time 3.00 minutes). An injection volume of 0.5 μl was used.

### Method 24 (K91 5 Min)

The HPLC measurement was performed using Waters Acquity H Class UPLC comprising a quaternary pump with degasser, a sample manager, a column oven (set at 40° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 50×2.1 mm) with a flow rate of 0.60 ml/min. Two mobile phases were used, mobile phase A: 5 Mm NH4oAc in water; mobile phase B: 5 Mm NH4oAc in ACN:Water (90:10)], and they were employed to run a gradient conditions from 5% B for 0.75 minutes, from 5% to 15% in 0.50 minutes, from 15% to 70% in 1.25 minutes, from 70% to 98% in 1.25 minutes, 98% B for 0.50 minutes and 5% B in 0.25 minutes and hold these conditions for 0.60 minutes in order to re-equilibrate the column (Total Run Time 5.10 minutes). An injection volume of 0.5 μl was used.

### Method 25 (K92-3 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 KV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 50×2.1 mm) with a flow rate of 0.600 ml/min. Two mobile phases were used, mobile phase A: 0.05% HCOOH in water; mobile phase B: 0.05% HCOOH in ACN:Water (90:10)], and they were employed to run a gradient conditions from 10% B for 0.75 minutes, from 10% to 50% in 0.25 minutes, and from 50% to 98% in 1.00 minutes, 98% B for 0.25 minutes and then 10% B in 0.35 minutes and hold these conditions for 0.40 minutes in order to re-equilibrate the column (Total Run Time 3.00 minutes). An injection volume of 0.5 μl was used.

### Method 26 (TM)

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems API2000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Gemini NX C18 (5 μm, 100×4.6 mm) with a flow rate of 1.00 ml/min. Two mobile phases were used, mobile phase A: 0.1% NH3 in Water); mobile phase B: ACN, and they were employed to run a gradient condition from 2% B for 1.50 min, from 2% B to 40% B in 3.50 minutes, and 40% B to 95% B in 3.00 minutes and 95% B for 6.00 minutes and 2% B in 1.00 minutes and hold these conditions for 4.00 minutes. Pre run Equilibration Time 4.00 min (Total Run Time 19.00 minutes). An injection volume of 1 μl to 3 μl was used (Depending on the sample concentration).

### Method 27 (K92-3 Min-YMC)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 to 3 μl was used (Depending on the sample concentration).

### Method 28 (K92-5 Min-YMC)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 5% B for 0.75 minutes, from 5% to 25% in 0.75 minutes, and from 25% to 95% in 1.50 minutes, 95% B for 1.75 minutes and then 5% B in 0.25 minutes and hold these conditions for 0.10 minutes in order to re-equilibrate the column (Total Run Time 5.10 minutes). An injection volume of 0.5 to 3 μl was used (Depending on the sample concentration).

### Method 29 (K70/71/55/63 5 Min-YMC)

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 5% B for 0.75 minutes, from 5% to 25% in 0.75 minutes, and from 25% to 95% in 1.50 minutes, 95% B for 1.75 minutes and then 5% B in 0.25 minutes and hold these conditions for 0.10 minutes in order to re-equilibrate the column (Total Run Time 5.10 minutes). An injection volume of 0.5 to 3 μl was used (Depending on the sample concentration).

### Method 30 (K71 5 Min-BEH)

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a Waters Acquity BEH C18 column (1.7 μm, 50×2.1 mm) with a flow rate of 0.50 ml/min. Two mobile phases were used, mobile phase A: 0.05% HCOOH in water; mobile phase B: 0.05% HCOOH in ACN:Water (90:10)], and they were employed to run a gradient conditions from 2% B for 0.50 minutes, from 2% to 30% in 1.00 minutes, and from 30% to 95% in 1.50 minutes, 95% B for 1.00 minutes and then 2% B in 0.50 minutes and hold these conditions for 0.60 minutes in order to re-equilibrate the column (Total Run Time 5.10 minutes). An injection volume of 0.5 to 3 μl was used (Depending on the sample concentration).

### Method 31 (K71 3 Min-BEH)

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a Waters Acquity BEH C18 column (1.7 μm, 50×2.1 mm) with a flow rate of 0.60 ml/min. Two mobile phases were used, mobile phase A: 0.05% HCOOH in water; mobile phase B: 0.05% HCOOH in ACN:Water (90:10)], and they were employed to run a gradient conditions from 5% B for 0.75 minutes, from 5% to 50% in 0.45 minutes, and from 50% to 98% in 0.80 minutes, 98% B for 0.25 minutes and then 5% B in 0.35 minutes and hold these conditions for 0.50 minutes in order to re-equilibrate the column (Total Run Time 3.10 minutes). An injection volume of 0.5 to 3 μl was used (Depending on the sample concentration).

### Method 32 (K103-3 Min)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters QDA) was configured with an electrospray ionization source. Mass spectra were acquired by scanning from 100 to 1000 in 0.20 second. The capillary needle voltage was 1.50 kV in positive and negative ionization mode and the source temperature was maintained at 150° C. Nitrogen was used as the desolvation gas, the flow was 600 L/Hour. Data acquisition was performed with Mass Lynx 4.2 Software. Reversed phase HPLC was carried out on a Waters Acquity BEH C18 column (1.7 μm, 50×2.1 mm) with a flow rate of 0.600 ml/min. Two mobile phases were used, mobile phase A: 0.05% HCOOH in water; mobile phase B: 0.05% HCOOH in ACN:Water (90:10)], and they were employed to run a gradient conditions from 10% B for 0.75 minutes, from 10% to 50% in 0.25 minutes, and from 50% to 98% in 1.00 minutes, 98% B for 0.25 minutes and then 10% B in 0.35 minutes and hold these conditions for 0.40 minutes in order to re-equilibrate the column (Total Run Time 3.00 minutes). An injection volume of 0.5 μl was used.

### NMR

All NMR spectra were obtained using Bruker Avance 400 MHZ spectrometers running Topspin Software.

### GCMS

### GCMS-Method-1:

GC-MS was taken on Agilent 6890 and 5973 N MSD series instrument.
Column: HP-5 MS (30×250 μm×0.25 μm)

### Carrier Gas: Helium

### Inlet Temperature: 250° C.

Split ratio: 5:1
Carrier Gas flow: 1.0 ml/min

### Solvent Delay: 3 min

Mass range: 50 to 550 amu
Injection volume: 1 μl

### Ramp Profile: -

Oven temperature initial from 100° C. held for 2 min then, 310° C. increasing at the rate of 35° C. held for 6 min. Total run time is 14 min.

### GCMS-Method-2:

GC-MS was taken on Agilent 7890B and 5977B MSD series instrument.
Column: HP-5 MS (30×250 μm×0.25 μm)

### Carrier Gas: Helium

### Inlet Temperature: 250° C.

Split ratio: 20:1
Carrier Gas flow: 1.0 ml/min

### Ramp Profile:

Oven temperature initial from 60° C. held for 2 min then, 100° C. increasing at the rate of 20° C. held for 2 min, 310° C. increasing at the rate of 40° C. held for 4 min. Total run time is 15.25 min.

### SFC

Supercritical fluid chromatography (SFC) analysis was performed on a WATERS SFC-analytical instrument. Column: Chiralpak IG 250×4.6 mm, particle size 5 μm. Method: mobile phase: A: carbon dioxide, mobile phase B: Hexane/IPA/Methanol 2/1/1 (0.3% Isopropyl amine), with isocratic flow 4.0 mL/min; 20% of B; wavelength: 240 nm.

### Example 1

### 5-chloro-N-methyl-N-(2,2,2-trifluoro-1-(4-fluorophenyl)ethyl)thiophene-2-sulfonamide

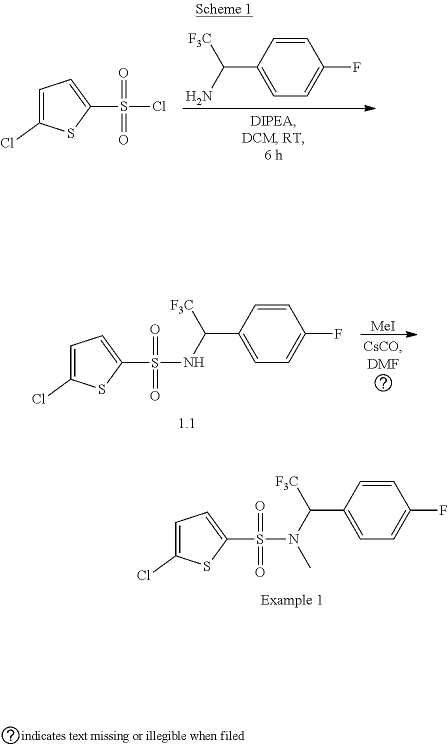 [SMILES: "MeI" ; O=S(=O)(Cl)c1ccc(Cl)s1 ; NC(c1ccc(F)cc1)C(F)(F)F ; O=S(=O)(NC(c1ccc(F)cc1)C(F)(F)F)c1ccc(Cl)s1 ; CN(C(c1ccc(F)cc1)C(F)(F)F)S(=O)(=O)c1ccc(Cl)s1]

### General Method for Sulfonamide Formation (Method A):

To a suspension of 2,2,2-trifluoro-1-(4-fluorophenyl) ethan-1-amine hydrochloride (528.8 mg, 2.3 mmol) in DCM (5 mL), DIPEA was added (1.2 mL, 6.9 mmol) and the solution was cooled at 0° C. A solution of 5-chlorothiophene-2-sulfonyl chloride (500 mg, 2.3 mmol) in DCM (0.3 mL) was added to it. The reaction mixture was stirred at RT for 1 h. The volatiles were evaporated under reduced pressure and the crude was purified by column chromatography over silica gel using 15% ethyl acetate in hexane and 1.1 was isolated as colorless sticky gum (140 mg, 16% yield). ¹H NMR (400 MHZ, DMSO-d₆) ä 9.68 (d, J=9.96 Hz, 1H), 7.55-7.49 (m, 2H), 7.31 (d, J=3.96 Hz, 1H), 7.14 (t, J=8.76 Hz, 2H), 7.04 (d, J=3.96 Hz, 1H), 5.44-5.39 (m, 1H).

### 5-chloro-N-methyl-N-(2,2,2-trifluoro-1-(4-fluorophenyl)ethyl)thiophene-2-sulfonamide (Example 1)

### General Method for Sulfonamide N-Alkylation (Method B):

To a stirred solution of a secondary sulfonamide (0.307 mmol) and Mel (3.067 mmol) in DMF (2 ml) K₂CO₃ or Cs₂CO₃ was added (0.46 mmol) and the RM was stirred at RT for 8 h. The reaction was diluted with cold water (10 mL) and extracted with ethyl acetate (2×10 mL). The combined organic part was washed with water (10 ml) and brine (10 ml), dried (anhydrous Na₂SO₄) and concentrated under reduced pressure to afford crude. The crude was further purified by column chromatography over silica gel using 25% ethyl acetate in hexane and the desired compound

The methylation of intermediate 1.1 was performed following the protocol as described in Method B using Cs₂CO₃ at 70° C. The compound was purified by column chromatography over silica gel using 20% ethyl acetate in hexane and Example 1 was isolated as light-yellow gum (22 mg, 42% yield, 95.04% purity). ¹H NMR (400 MHZ, DMSO-d₆) ä 7.71 (d, J=4.1 Hz, 1H), 7.44 (dd, J=5.4, 8.6 Hz, 2H), 7.32 (d, J=4.0 Hz, 1H), 7.26 (t, J=8.8 Hz, 2H), 6.00 (q, J=8.6 Hz, 1H), 2.78 (s, 3H).

### Example 2 to Example 7

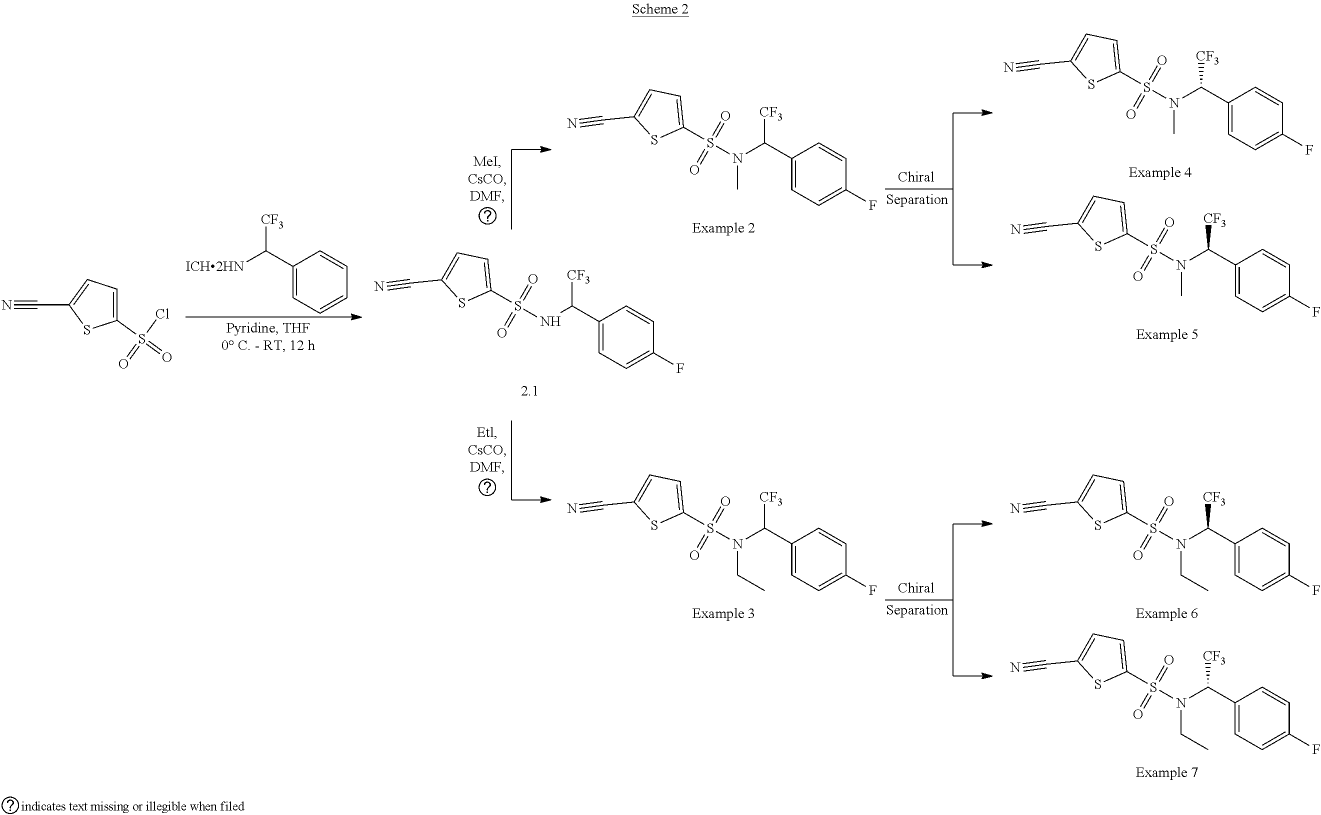

### 5-cyano-N-(2,2,2-trifluoro-1-(4-fluorophenyl)ethyl)thiophene-2-sulfonamide 2.1

Synthesis of intermediate 2.1 was performed essentially as described in Method A using 2,2,2-trifluoro-1-(4-fluorophenyl) ethan-1-amine hydrochloride and 5-cyanothiophene-2-sulfonyl chloride. Pyridine was used as base and THE as solvent. The compound was purified by column chromatography over silica gel using 40% ethyl acetate:hexane and isolated as yellow solid (90 mg, 51% yield). ¹H NMR (400 MHZ, DMSO-d6): δ 10.10-9.94 (m, 1H), 7.82 (d, J=4.0 Hz, 1H), 7.54-7.46 (m, 4H), 7.15 (t, J=8.8 Hz, 2H), 5.59-5.43 (m, 1H).

### Example 2

### 5-cyano-N-methyl-N-(2,2,2-trifluoro-1-(4-fluorophenyl)ethyl)thiophene-2-sulfonamide

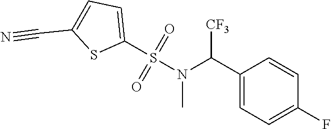 [SMILES: CN(C(c1ccc(F)cc1)C(F)(F)F)S(=O)(=O)c1ccc(C#N)s1]

The methylation of intermediate 2.1 was preformed following the protocol as described in Method B using Cs₂CO₃ as base at 70° C. The crude was purified by column chromatography over silica gel using 35% ethyl acetate in hexane and compound Example-2 was isolated as yellow oil (15 mg, 48% yield, 99.40% purity). ¹H NMR (400 MHZ, DMSO-d₆): δ 8.09 (d, J=4.0 Hz, 1H), 7.92 (d, J=4.0 Hz, 1H), 7.43 (dd, J=5.3, 8.6 Hz, 2H), 7.27 (t, J=8.8 Hz, 2H), 6.07 (q, J=8.4 Hz, 1H), 2.82 (s, 3H).

### Example 3

### 5-cyano-N-ethyl-N-(2,2,2-trifluoro-1-(4-fluorophenyl)ethyl)thiophene-2-sulfonamide

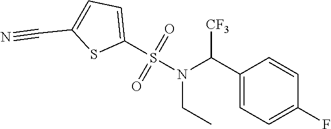 [SMILES: CCN(C(c1ccc(F)cc1)C(F)(F)F)S(=O)(=O)c1ccc(C#N)s1]

The ethylation of intermediate 2.1 was performed following the protocol as described in Method B using Cs₂CO₃ as base at 70° C. The crude was purified by column chromatography over silica gel using 35% ethyl acetate in hexane and compound Example-3) was isolated as yellow oil (15 mg, 46% yield, 99.73% purity). GCMS: m/z found 392.1 (CHE 300-METHOD-1); ¹H NMR (400 MHZ, DMSO-d₆): δ 8.06 (d, J=4.0 Hz, 1H), 7.92 (d, J=4.0 Hz, 1H), 7.44 (t, J=5.3 Hz, 2H), 7.27 (t, J=8.8 Hz, 2H), 6.05 (q, J=8.6 Hz, 1H), 3.45-3.31 (m, 2H), 1.01 (t, J=6.8 Hz, 3H).

### Examples 4 and 5

### (S)-5-cyano-N-ethyl-N-(2,2,2-trifluoro-1-(4-fluorophenyl)ethyl)thiophene-2-sulfonamide (Example 4) and (R)-5-cyano-N-ethyl-N-(2,2,2-trifluoro-1-(4-fluorophenyl)ethyl)thiophene-2-sulfonamide (Example 5

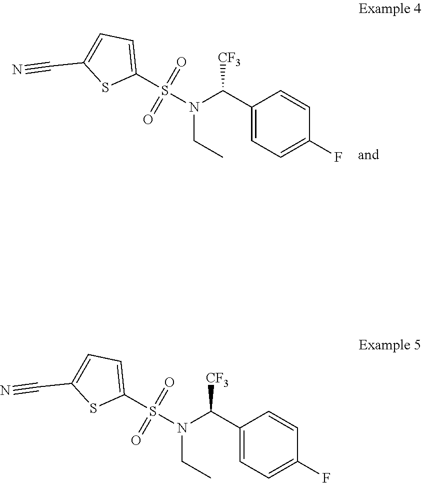 [SMILES: CCN([C@@H](c1ccc(F)cc1)C(F)(F)F)S(=O)(=O)c1ccc(C#N)s1 ; CCN([C@H](c1ccc(F)cc1)C(F)(F)F)S(=O)(=O)c1ccc(C#N)s1]

The chiral separation of 5-cyano-N-ethyl-N-(2,2,2-trifluoro-1-(4-fluorophenyl)ethyl)thiophene-2-sulfonamide (Example 3) provided both the enantiomer as mentioned below. Chiral Separation Method 1: CHIRALPAK IA (250×20 mm) 5u, Flow rate-18 ml/min, Mobile phase-hexane/ETOH-95/05, Solubility-DCM, Wavelength-268 nm, Run time-22 min.

(Example 4): Yellow oil (4 mg, 99.41% purity). GCMS: m/z found 392.0 (CHE 300-METHOD-3); ¹H NMR (400 MHZ, DMSO-d₆): δ 8.06 (d, J=4.0 Hz, 1H), 7.92 (d, J=4.0 Hz, 1H), 7.45 (dd, J=5.2, 8.6 Hz, 2H), 7.27 (t, J=8.8 Hz, 2H), 6.05 (q, J=8.7 Hz, 1H), 3.48-3.33 (m, 2H), 1.01 (t, J=7.0 Hz, 3H).

(Example 5): Yellow oil (5 mg, 99.15% purity). GCMS: m/z found 392.0 (CHE 300-METHOD-3); ¹H NMR (400 MHZ, DMSO-d₆): δ 8.06 (d, J=4.0 Hz, 1H), 7.92 (d, J=4.0 Hz, 1H), 7.45 (dd, J=5.3, 8.6 Hz, 2H), 7.27 (t, J=8.8 Hz, 2H), 6.04 (q, J=8.6 Hz, 1H), 3.47-3.31 (m, 2H), 1.01 (t, J=7.0 Hz, 3H).

### Examples 6 and 7

### (R)-5-cyano-N-methyl-N-(2,2,2-trifluoro-1-(4-fluorophenyl)ethyl)thiophene-2-sulfonamide (Example 6) and(S)-5-cyano-N-methyl-N-(2,2,2-trifluoro-1-(4-fluorophenyl)ethyl)thiophene-2-sulfonamide (Example 7)

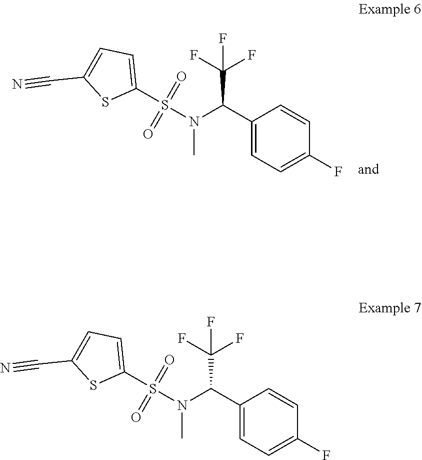 [SMILES: CN([C@H](c1ccc(F)cc1)C(F)(F)F)S(=O)(=O)c1ccc(C#N)s1 ; CN([C@@H](c1ccc(F)cc1)C(F)(F)F)S(=O)(=O)c1ccc(C#N)s1]

The chiral separation of 5-cyano-N-methyl-N-(2,2,2-trifluoro-1-(4-fluorophenyl)ethyl)thiophene-2-sulfonamide (Example 2) provided both the enantiomer as mentioned below. Chiral Separation Method: CHIRALPAK IG (250×21 mm) 5u, Flow rate—21 ml/min, Mobile phase—HEXANE/MEOH/MTBE—96/02/02, Solubility—DCM, Wavelength—268 nm, Run time—25 min.

(Example 6): Yellow oil (4 mg, 99.35% purity). GCMS: m/z found 377.9 (CHE 300-METHOD-2); ¹H NMR (400 MHz, DMSO-d₆): δ 8.08 (d, J=4.0 Hz, 1H), 7.92 (d, J=4.0 Hz, 1H), 7.43 (dd, J=5.3, 8.6 Hz, 2H), 7.26 (t, J=8.7 Hz, 2H), 6.07 (q, J=8.5 Hz, 1H), 2.82 (s, 3H).

(Example 7): Yellow oil (4 mg, 98.51% purity). GCMS: m/z found 378.1 (CHE 300-METHOD-2); ¹H NMR (400 MHz, DMSO-d₆): δ 8.09 (d, J=4.0 Hz, 1H), 7.92 (d, J=4.1 Hz, 1H), 7.43 (dd, J=5.3, 8.6 Hz, 2H), 7.26 (t, J=8.8 Hz, 2H), 6.07 (q, J=8.6 Hz, 1H), 2.82 (s, 3H).

### Example 8 to Example 13

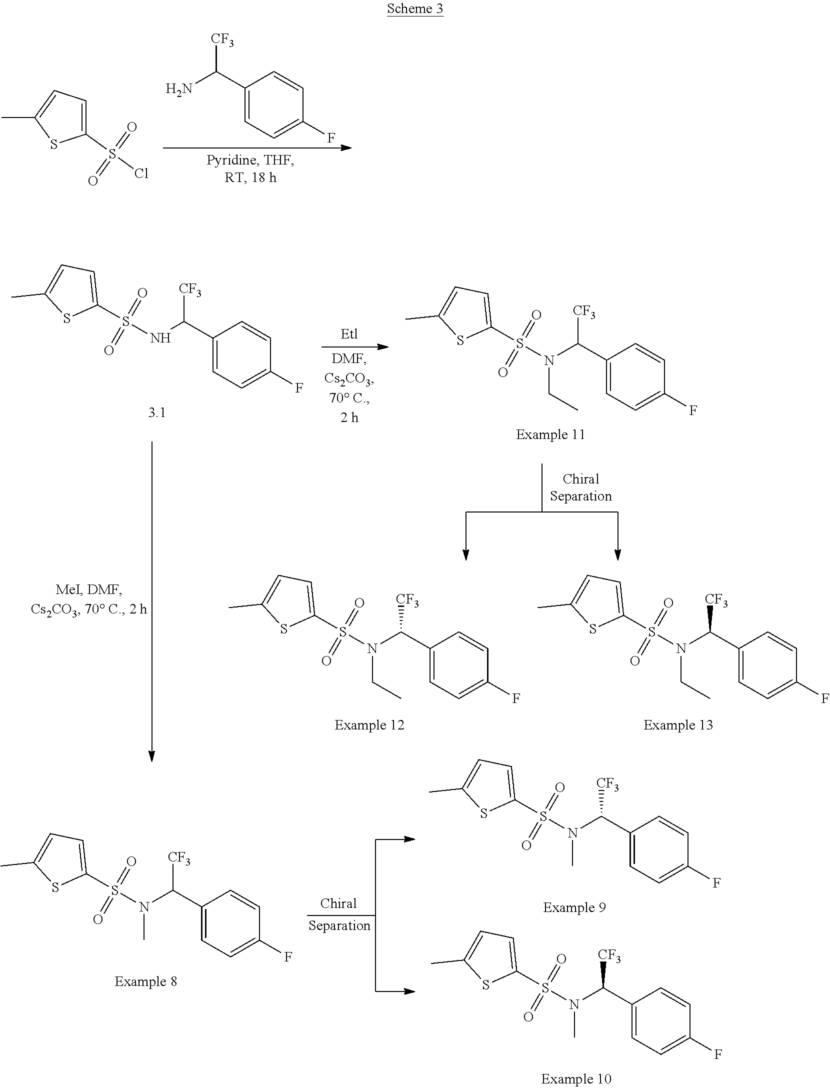

5-methyl-N-(2,2,2-trifluoro-1-(4-fluorophenyl)ethyl)thiophene-2-sulfonamide 3.1: Intermediate 3.1 was synthesized from 5-methylthiophene-2-sulfonyl chloride following the procedure described in Method A using 2,2,2-trifluoro-1-(4-fluorophenyl)ethan-1-amine and pyridine as base. The compound was purified by flash chromatography over silica gel using 20% ethyl acetate in hexane and isolated as light-yellow oil (100 mg, 56% yield). ¹H NMR (400 MHz, DMSO-d₆) δ 9.41 (d, J=9.9 Hz, 1H), 7.50 (t, J=8.2 Hz, 2H), 7.22 (d, J=3.7 Hz, 1H), 7.12 (t, J=8.7 Hz, 2H), 6.67 (d, J=3.4 Hz, 1H), 5.56-5.03 (m, 1H), 2.37 (s, 3H).

### Example 8

### N-ethyl-5-methyl-N-(2,2,2-trifluoro-1-(4-fluorophenyl)ethyl)thiophene-2-sulfonamide

The ethylation of intermediate 3.1 was performed following the protocol as described in Method B using Cs₂CO₃ as base at 70° C. The crude was purified by column chromatography over silica gel using 35% ethyl acetate in hexane and the desired compound Example 8 was isolated as light-yellow gum (15 mg, 55% yield, 98.88% purity). LCMS: m/z found 382.1[M+H]⁺, rt 3.85 min (Method 1) [Xbridge C18 column (5 μm, 50×4.6 mm)]; ¹H NMR (400 MHz, Chloroform-d) δ 7.42 (d, J=3.7 Hz, 1H), 7.39 (dd, J=5.2, 8.6 Hz, 2H), 7.11-7.00 (m, 2H), 6.74 (dd, J=1.1, 3.8 Hz, 1H), 5.74 (q, J=8.4 Hz, 1H), 3.40-3.26 (m, 1H), 3.21-3.07 (m, 1H), 2.52 (s, 3H), 1.00 (t, J=7.0 Hz, 3H).

### (S)—N-ethyl-5-methyl-N-(2,2,2-trifluoro-1-(4-fluorophenyl)ethyl)thiophene-2-sulfonamide (Example 9) and (R)—N-ethyl-5-methyl-N-(2,2,2-trifluoro-1-(4-fluorophenyl)ethyl)thiophene-2-sulfonamide (Example 10

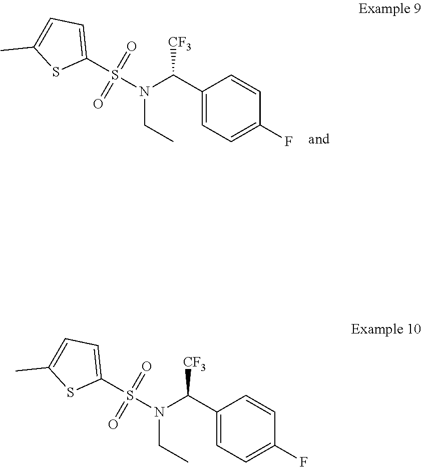 [SMILES: CCN([C@@H](c1ccc(F)cc1)C(F)(F)F)S(=O)(=O)c1ccc(C)s1 ; CCN([C@H](c1ccc(F)cc1)C(F)(F)F)S(=O)(=O)c1ccc(C)s1]

Chiral separation of N-ethyl-5-methyl-N-(2,2,2-trifluoro-1-(4-fluorophenyl)ethyl)thiophene-2-sulfonamide provided both the enantiomers as mentioned below. Chiral Separation Method: Chiral separation was done on Agilent 1200 series instrument. Column name: Chiralcel OJ-H (250×21 mm), 5μ, operating at ambient temperature and flow rate of 21.0 mL/min. Mobile phase: The mixture of 95% Hexane and 5% Ethanol, held this isocratic mixture up to 35 min with wavelength of 264 nm.

Example 9: Colourless sticky solid (10 mg, 99.26% purity). LCMS: m/z found 382.1 [M+H]⁺, rt=3.73 min (Method 1) [Xbridge C8 column (5 μm, 50×4.6 mm)]; ¹H NMR (400 MHz, Chloroform-d) δ 7.42 (d, J=3.8 Hz, 1H), 7.39 (dd, J=5.2, 8.6 Hz, 2H), 7.06 (t, J=8.6 Hz, 2H), 6.74 (dd, J=1.2, 3.7 Hz, 1H), 5.74 (q, J=8.5 Hz, 1H), 3.39-3.26 (m, 1H), 3.21-3.07 (m, 1H), 2.52 (s, 3H), 1.00 (t, J=7.0 Hz, 3H).

Example 10: Colourless sticky solid (10 mg, 98.33%). LCMS: m/z found 382.2 [M+H]⁺, rt=3.74 min (Method 1) [Xbridge C8 column (5 μm, 50×4.6 mm)]. ¹H NMR (400 MHz, Chloroform-d) δ 7.43 (d, J=3.7 Hz, 1H), 7.39 (dd, J=5.1, 8.4 Hz, 2H), 7.06 (t, J=8.6 Hz, 1H), 6.73 (d, J=3.8 Hz, 1H), 5.74 (q, J=8.3 Hz, 1H), 3.40-3.26 (m, 1H), 3.21-3.07 (m, 1H), 2.52 (s, 3H), 1.00 (t, J=7.1 Hz, 3H).

### Example 11

### N,5-dimethyl-N-(2,2,2-trifluoro-1-(4-fluorophenyl)ethyl)thiophene-2-sulfonamide

The methylation of intermediate 3.1 was performed following the protocol as described in Method B using Cs₂CO₃ as base at 70° C. The crude was purified by column chromatography over silica gel using 35% ethyl acetate in hexane and the desired compound Example 11 was isolated as light-yellow gum (15 mg, 57% yield, 96.26% purity). LCMS: m/z found 368.0 [M+H]⁺, rt=3.72 min (Method 1) [Xbridge C18 column (5 μm, 50×4.6 mm)]; ¹H NMR (400 MHz, Chloroform-d) δ 7.41 (d, J=3.7 Hz, 1H), 7.37 (dd, J=5.1, 8.5 Hz, 2H), 7.07 (t, J=8.6 Hz, 2H), 6.78-6.72 (m, 1H), 5.80 (q, J=8.2 Hz, 1H), 2.72 (s, 3H), 2.52 (s, 3H).

### Examples 12 and 13

### (S)—N,5-dimethyl-N-(2,2,2-trifluoro-1-(4-fluorophenyl)ethyl)thiophene-2-sulfonamide (Example 12) and (R)—N,5-dimethyl-N-(2,2,2-trifluoro-1-(4-fluorophenyl)ethyl)thiophene-2-sulfonamide (Example 13)

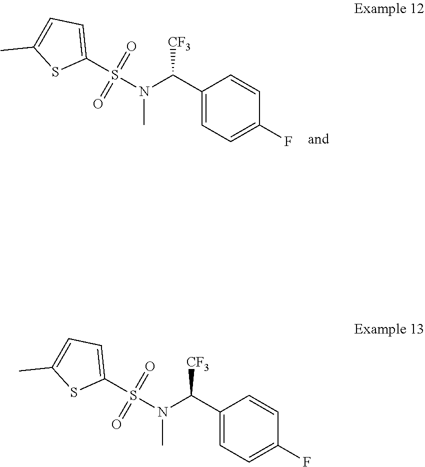 [SMILES: Cc1ccc(S(=O)(=O)N(C)[C@@H](c2ccc(F)cc2)C(F)(F)F)s1 ; Cc1ccc(S(=O)(=O)N(C)[C@H](c2ccc(F)cc2)C(F)(F)F)s1]

Chiral separation of N,5-dimethyl-N-(2,2,2-trifluoro-1-(4-fluorophenyl)ethyl)thiophene-2-sulfonamide provided both the enantiomers as mentioned below. Chiral separation Method: Chiral separation was done on Agilent 1200 series instrument. Column name: Chiralcel OJ-H (250×21 mm), 5μ, operating at ambient temperature and flow rate of 21.0 mL/min. Mobile phase: The mixture of 95% Hexane and 5% Ethanol, held this isocratic mixture up to 20 min with wavelength of 264 nm.

(Example-12): Colourless sticky solid (10 mg, 99.09% purity). LCMS: m/z found 367.9 [M+H]⁺, rt=3.64 min (Method 1) [Xbridge C8 column (5 μm, 50×4.6 mm)]; ¹H NMR (400 MHz, Chloroform-d) δ 7.42 (d, J=3.6 Hz, 1H), 7.37 (dd, J=5.2, 8.5 Hz, 2H), 7.07 (t, J=8.8 Hz, 2H), 6.74 (d, J=3.6 Hz, 1H), 5.79 (q, J=8.4 Hz, 1H), 2.72 (s, 3H), 2.52 (s, 3H).

(Example-13): Colourless sticky solid (10 mg, 99.63% purity). LCMS: m/z found 368.0[M+H]⁺, rt 3.74 min (Method 1) [Xbridge C8 column (5 μm, 50×4.6 mm)]; ¹H NMR (400 MHz, Chloroform-d) δ 7.41 (d, J=4.0 Hz, 1H), 7.37 (dd, J=5.2, 8.4 Hz, 2H), 7.07 (t, J=8.4 Hz, 2H), 6.78-6.72 (m, 1H), 5.79 (q, J=8.4 Hz, 1H), 2.72 (s, 3H), 2.52 (s, 3H).

### Example 14 to Example 16

### N-ethyl-5-fluoro-N-(2,2,2-trifluoro-1-(4-fluorophenyl)ethyl)thiophene-2-sulfonamide (Example 14), (S)—N-ethyl-5-fluoro-N-(2,2,2-trifluoro-1-(4-fluorophenyl)ethyl)thiophene-2-sulfonamide (Example 15) and (R)—N-ethyl-5-fluoro-N-(2,2,2-trifluoro-1-(4-fluorophenyl)ethyl)thiophene-2-sulfonamide (Example 16)

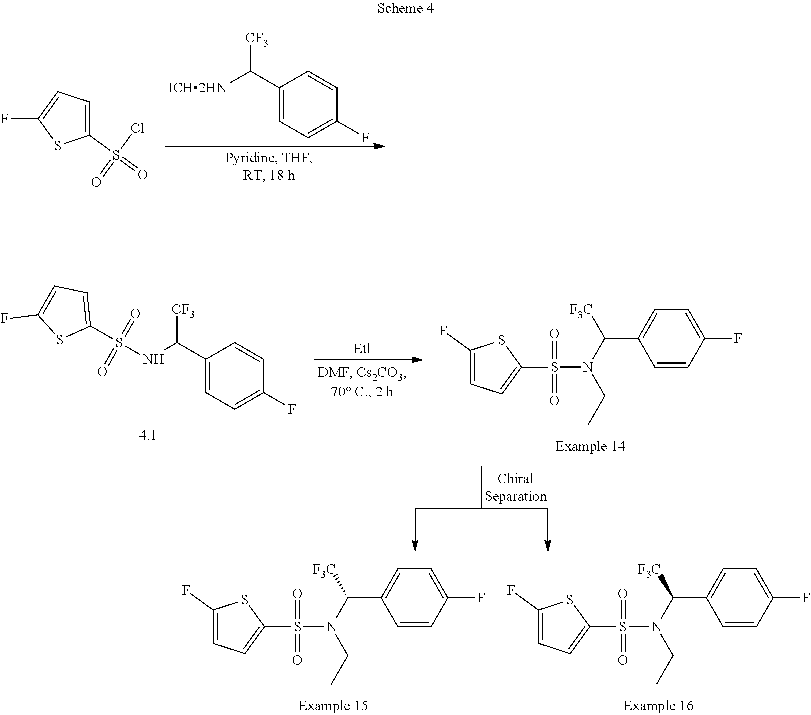

5-fluoro-N-(2,2,2-trifluoro-1-(4-fluorophenyl)ethyl)thiophene-2-sulfonamide 4.1: Intermediate 4.1 was synthesized from 5-fluorothiophene-2-sulfonyl chloride following the procedure described in Method A using 2,2,2-trifluoro-1-(4-fluorophenyl)ethan-1-amine hydrochloride. The compound was purified by flash chromatography over silica gel using 20% ethyl acetate in hexane and isolated as colourless oil (300 mg, 42% yield). ¹H NMR (400 MHz, DMSO-d₆) δ 9.62 (d, J=9.0 Hz, 1H), 7.52 (dd, J=5.4, 8.5 Hz, 2H), 7.21 (t, J=4.0 Hz, 1H), 7.16 (t, J=8.8 Hz, 2H), 6.69 (d, J=4.3 Hz, 1H), 5.39 (t, J=8.0 Hz, 1H).

### Example 14

### N-ethyl-5-fluoro-N-(2,2,2-trifluoro-1-(4-fluorophenyl)ethyl)thiophene-2-sulfonamide

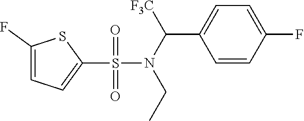 [SMILES: CCN(C(c1ccc(F)cc1)C(F)(F)F)S(=O)(=O)c1ccc(F)s1]

The ethylation of intermediate 4.1 was preformed following the protocol as described in Method B using Cs₂CO₃ as base at 70° C. The compound Example-14 was purified by column chromatography over silica gel using 20% ethyl acetate in hexane and isolated as light-yellow oil (25 mg, 50% yield, 93.93% purity). ¹H NMR (400 MHz, DMSO-d₆): δ 7.62 (t, J=4.0 Hz, 1H), 7.44 (dd, J=5.4, 8.6 Hz, 2H), 7.27 (t, J=8.7 Hz, 2H), 6.96 (dd, J=1.5, 4.3 Hz, 1H), 5.95 (q, J=8.6 Hz, 1H), 3.30-3.15 (m, 2H), 0.99 (t, J=7.0 Hz, 3H).

Chiral separation of racemic Example 14 provided both the enantiomers as mentioned below. Chiral separation method: Chiral separation was done on Agilent 1200 series instrument. Column name: Chiralcel OJ-H (250×20 mm), 5μ, operating at ambient temperature and flow rate of 18.0 mL/min. Mobile phase: 0.1% Isopropyl amine in the mixture of 85% Hexane and 15% Ethanol, held this isocratic mixture up to 30 min with wavelength of 258 nm.

Example 15: Pale yellow liquid (20 mg, 98.35% Purity). ¹H NMR (400 MHz, DMSO-d₆): δ 7.62 (t, J=4.1 Hz, 1H), 7.44 (dd, J=5.3, 8.6 Hz, 2H), 7.27 (t, J=8.8 Hz, 2H), 6.96 (dd, J=1.6, 4.4 Hz, 1H), 5.95 (q, J=8.4 Hz, 1H), 3.40-3.29 (m, 1H), 3.27-3.17 (m, 1H), 0.99 (t, J=7.0 Hz, 3H).

Example 16: Light yellow oil (20 mg, 97.94% Purity). ¹H NMR (400 MHz, DMSO-d₆): δ 7.62 (t, J=4.0 Hz, 1H), 7.45 (dd, J=5.3, 8.6 Hz, 2H), 7.28 (t, J=8.8 Hz, 2H), 6.96 (dd, J=1.6, 4.3 Hz, 1H), 5.95 (q, J=8.8 Hz, 1H), 3.42-3.31 (m, 1H), 3.27-3.15 (m, 1H), 1.00 (t, J=7.2 Hz, 3H).

### Example 17

### 5-fluoro-N-methyl-N-(2,2,2-trifluoro-1-(4-fluorophenyl)ethyl)thiophene-2-sulfonamide

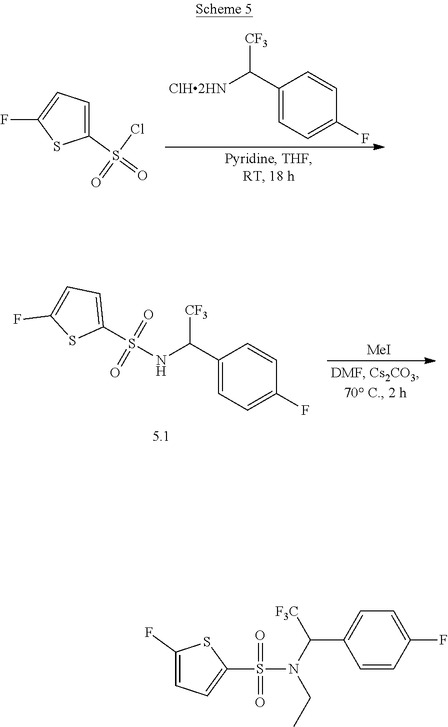

5-fluoro-N-(2,2,2-trifluoro-1-(4-fluorophenyl)ethyl)thiophene-2-sulfonamide (5.1): Intermediate 5.1 was synthesized from 5-fluorothiophene-2-sulfonyl chloride following the procedure described in Method A using 2,2,2-trifluoro-1-(4-fluorophenyl)ethan-1-amine hydrochloride. Pyridine was used as base in THF solvent. The compound was purified by flash chromatography over silica gel using 20% ethyl acetate in hexane and isolated as colourless oil (300 mg, 42% yield). ¹H NMR (400 MHz, DMSO-d₆): δ 9.78-9.53 (m, 1H), 7.58-7.46 (m, 2H), 7.25-7.08 (m, 4H), 5.54-5.29 (m, 1H).

5-fluoro-N-methyl-N-(2,2,2-trifluoro-1-(4-fluorophenyl)ethyl)thiophene-2-sulfonamide (Example 17): The methylation of intermediate 5.1 was performed following the protocol as described in Method B using Cs₂CO₃ as base at 70° C. The compound Example-17 was purified by column chromatography over silica gel using 20% ethyl acetate in hexane and isolated as colorless oil (20 mg, 43% yield, 99.40% purity). LCMS: m/z found 371.2 [M+H]⁺, rt=3.67 min (Method 1) [Xbridge C8 column (5 μm, 50×4.6 mm]. ¹H NMR (400 MHz, DMSO-d₆): δ 7.62 (t, J=4.1 Hz, 1H), 7.44 (dd, J=5.3, 8.6 Hz, 2H), 7.27 (t, J=8.8 Hz, 2H), 6.98 (dd, J=1.6, 4.4 Hz, 1H), 6.00 (q, J=8.4 Hz, 1H), 2.76 (s, 3H).

### Example 18 and Example 19

### 5-fluoro-N-methyl-N-(2,2,2-trifluoro-1-(4-fluorophenyl)ethyl)thiophene-2-sulfonamide (Example 18) and 5-fluoro-N-methyl-N-(2,2,2-trifluoro-1-(4-fluorophenyl)ethyl)thiophene-2-sulfonamide (Example 19)

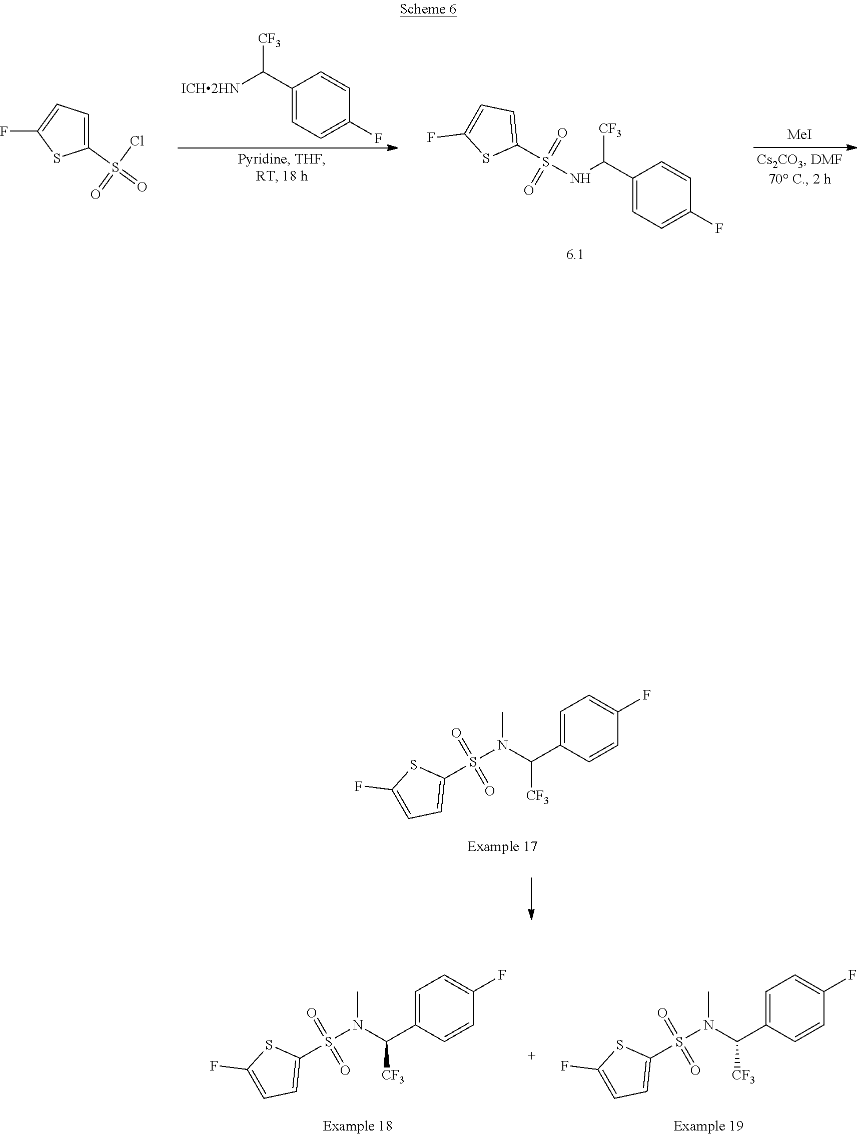

5-fluoro-N-(2,2,2-trifluoro-1-(4-fluorophenyl)ethyl)thiophene-2-sulfonamide 6.1: Intermediate 6.1 was synthesized from 5-fluorothiophene-2-sulfonyl chloride following the procedure described in Method A using 2,2,2-trifluoro-1-(4-fluorophenyl)ethan-1-amine hydrochloride. The compound was purified by flash chromatography over silica gel using 20% ethyl acetate in hexane and isolated as colourless oil (300 mg, 42% yield). ¹H NMR (400 MHz, DMSO-d₆) δ 9.62 (d, J=9.0 Hz, 1H), 7.52 (dd, J=5.4, 8.5 Hz, 2H), 7.21 (t, J=4.0 Hz, 1H), 7.16 (t, J=8.8 Hz, 2H), 6.69 (d, J=4.3 Hz, 1H), 5.39 (t, J=8.0 Hz, 1H).

Chiral separation of racemic compound Example 17 provided both the enantiomers as mentioned below. Chiral separation method: Chiral separation was done on Agilent 1200 series instrument. Column name: Chiralcel OJ-H (250×20 mm), 5μ, operating at ambient temperature and flow rate of 18 mL/min. Mobile phase: The mixture of 98% hexane and 2% ethanol, held this isocratic mixture up to 25 min with wavelength of 256 nm.

Example 18: Colourless sticky gum (10 mg, 99.35% purity). GCMS: m/z found 371.1 (CHE 300-METHOD) (HP-5MS (30×250 μm×0.25 μm); ¹H NMR (400 MHz, DMSO-d₆): δ 7.62 (t, J=4.0 Hz, 1H), 7.43 (dd, J=5.3, 8.6 Hz, 2H), 7.27 (t, J=8.8 Hz, 2H), 6.99 (dd, J=1.6, 4.4 Hz, 1H), 5.98 (q, J=8.6 Hz, 1H), 2.76 (s, 3H).

Example 19: Colourless sticky gum (10 mg, 99.35% purity). GCMS: m/z found 371.1 (CHE 300-METHOD) (HP-5MS (30×250 μm×0.25 μm); ¹H NMR (400 MHz, DMSO-d₆): δ 7.62 (t, J=4.1 Hz, 1H), 7.43 (dd, J=5.4, 8.7 Hz, 2H), 7.27 (t, J=8.8 Hz, 2H), 6.99 (dd, J=1.5, 4.4 Hz, 1H), 5.99 (q, J=8.6 Hz, 1H), 2.76 (s, 3H).

### Example 20

### N-ethyl-1-(tetrahydro-2H-pyran-4-yl)-N-(2,2,2-trifluoro-1-(4-fluorophenyl)ethyl)methanesulfonamide

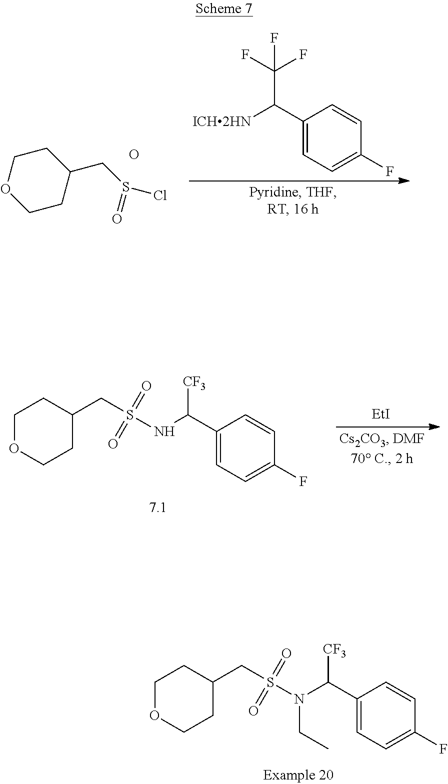

### 1-(tetrahydro-2H-pyran-4-yl)-N-(2,2,2-trifluoro-1-(4-fluorophenyl)ethyl)methanesulfonamide 7.1

Intermediate 7.1 was synthesized from (tetrahydro-2H-pyran-4-yl)methanesulfonyl chloride following the protocol as described in Method A using 2,2,2-trifluoro-1-(4-fluorophenyl)ethan-1-amine. Pyridine was used as base and THF as solvent. The compound was purified by column chromatography over silica gel using 25% ethyl acetate in hexane and isolated as colourless oil (45 mg, 50% yield). ¹H NMR (400 MHz, DMSO-d₆): δ 8.89 (d, J=9.7 Hz, 1H), 7.69 (d, J=7.9 Hz, 2H), 7.30 (d, J=7.7 Hz, 2H), 5.50-5.17 (m, 1H), 3.91-3.49 (m, 2H), 3.23-2.67 (m, 4H), 1.86-1.55 (m, 2H), 1.36-0.97 (m, 3H).

### N-ethyl-1-(tetrahydro-2H-pyran-4-yl)-N-(2,2,2-trifluoro-1-(4-fluorophenyl)ethyl)methanesulfonamide

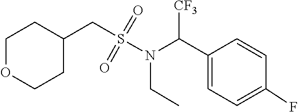 [SMILES: CCN(C(c1ccc(F)cc1)C(F)(F)F)S(=O)(=O)CC1CCOCC1]

The ethylation of intermediate 7.1 was performed following the protocol as described in Method B at 70° C. The crude was purified by column chromatography over silica gel using 25% ethyl acetate in hexane and the desired compound Example 20 was isolated as colourless gum (19 mg, 45% yield, 98.81% purity). GCMS: m/z found 383.1 [CHE 300-METHOD: HP-5MS (30×250 μm×0.25 μm)]; ¹H NMR (400 MHz, DMSO-d₆): δ 7.62 (dd, J=5.3, 8.5 Hz, 2H), 7.33 (t, J=8.8 Hz, 2H), 5.81 (q, J=8.7 Hz, 1H), 3.81 (d, J=11.3 Hz, 2H), 3.29-3.05 (m, 4H), 2.18-1.97 (m, 1H), 1.83-1.63 (m, 3H), 1.42-1.27 (m, 3H), 0.95 (t, J=7.0 Hz, 3H).

### Example 21 and Example 22

### 5-cyano-N-ethyl-N-(2,2,2-trifluoro-1-(4-fluorophenyl)ethyl)thiophene-2-sulfonamide (Example 21) and 5-(N-ethyl-N-(2,2,2-trifluoro-1-(4-fluorophenyl)ethyl)sulfamoyl)thiophene-2-carboxamide (Example 22)

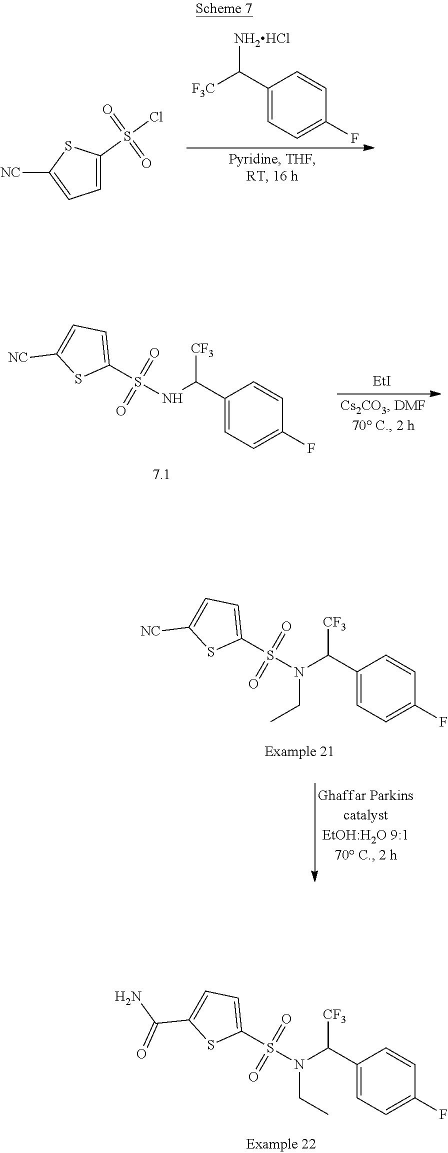

### 5-cyano-N-(1-(4-fluorophenyl)-2,2-dimethylpropyl)thiophene-2-sulfonamide 7.1

Synthesis of intermediate 7.1 was preformed essentially as described in Method A using 2,2,2-trifluoro-1-(4-fluorophenyl)ethan-1-amine hydrochloride and 5-cyanothiophene-2-sulfonyl chloride. Pyridine was used as base and THF as solvent. The compound was purified by column chromatography over silica gel using 40% ethyl acetate:hexane and isolated as yellow solid (90 mg, 51% yield). ¹H NMR (400 MHz, DMSO-d₆): δ 10.10-9.94 (m, 1H), 7.82 (d, J=4.0 Hz, 1H), 7.54-7.46 (m, 4H), 7.15 (t, J=8.8 Hz, 2H), 5.59-5.43 (m, 1H).

### 5-cyano-N-ethyl-N-(2,2,2-trifluoro-1-(4-fluorophenyl)ethyl)thiophene-2-sulfonamide (Example 21)

The ethylation of intermediate 7.1 was preformed following the protocol as described in Method B using Cs₂CO₃ as base at 70° C. The crude was purified by column chromatography over silica gel using 35% ethyl acetate in hexane and compound Example 21 was isolated as yellow oil (15 mg, 46% yield, 99.73% purity). GCMS: m/z found 392.1 (CHE 300-METHOD-1); ¹H NMR (400 MHz, DMSO-d₆): δ 8.06 (d, J=4.0 Hz, 1H), 7.92 (d, J=4.0 Hz, 1H), 7.44 (t, J=5.3 Hz, 2H), 7.27 (t, J=8.8 Hz, 2H), 6.05 (q, J=8.6 Hz, 1H), 3.45-3.31 (m, 2H), 1.01 (t, J=6.8 Hz, 3H).

### 5-(N-ethyl-N-(2,2,2-trifluoro-1-(4-fluorophenyl)ethyl)sulfamoyl)thiophene-2-carboxamide (Example 22)

Hydrolysis of nitriles: Method N: To a stirred solution of Example 21 (55 mg, 0.14 mmol) in ethanol:water (9:1) (2 mL) was added Ghaffar Parkins catalyst (6 mg, 0.014 mmol) and the reaction mixture was heated at 70° C. for 2 h. The volatiles were evaporated under reduced pressure and the crude product was purified by column chromatography using 50% ethyl acetate in hexane. The desired compound Example 22 was isolated as a white sticky solid (39 mg, 68% yield, 99.10% purity). LCMS: m/z found 409.11[M−H], rt=2.70 min (Method 4) [Xbridge C18 column (3.5 μm, 50×3 mm)]; ¹H NMR (400 MHz, DMSO-d₆) δ 8.27 (s, 1H), 7.79 (d, J=4.0 Hz, 1H), 7.74 (d, J=4.0 Hz, 1H), 7.43 (dd, J=5.3, 8.6 Hz, 2H), 7.25 (t, J=8.8 Hz, 2H), 5.97 (q, J=8.5 Hz, 1H), 3.43-3.32 (m, 1H), 3.30-3.17 (m, 1H), 1.00 (t, J=7.0 Hz, 3H).

### Example 23

### 5-chloro-N-(2,2-difluoro-1-(4-fluorophenyl)ethyl)-N-methylthiophene-2-sulfonamide

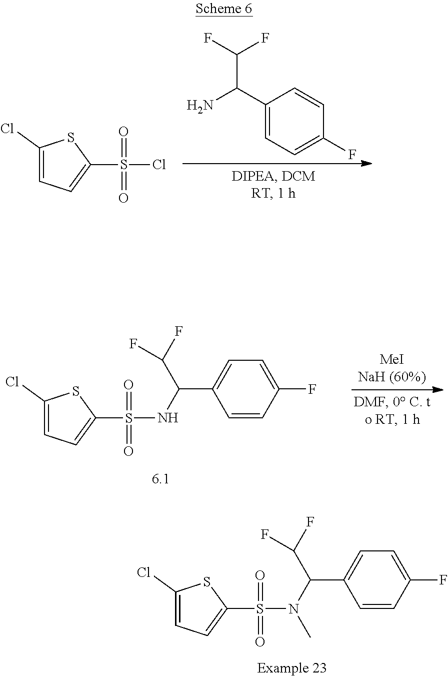 [SMILES: O=S(=O)(Cl)c1ccc(Cl)s1 ; NC(c1ccc(F)cc1)C(F)F ; O=S(=O)(NC(c1ccc(F)cc1)C(F)F)c1ccc(Cl)s1 ; "MeINaH (60%)" ; CN(C(c1ccc(F)cc1)C(F)F)S(=O)(=O)c1ccc(Cl)s1]

5-fluoro-N-(2,2,2-trifluoro-1-(4-fluorophenyl)ethyl)thiophene-2-sulfonamide (6.1): Intermediate 6.1 was synthesized from 5-fluorothiophene-2-sulfonyl chloride following the procedure described in Method A using 2,2,2-trifluoro-1-(4-fluorophenyl)ethan-1-amine hydrochloride. Pyridine was used as base and THF as a solvent. The compound was purified by flash chromatography over silica gel using 20% ethyl acetate in hexane and isolated as a colourless oil (300 mg, 42% yield). ¹H NMR (400 MHz, DMSO-d₆): δ 9.78-9.53 (m, 1H), 7.58-7.46 (m, 2H), 7.25-7.08 (m, 4H), 5.54-5.29 (m, 1H).

Methylation of intermediate 6.1 was performed following the protocol as described in Method B. The crude was purified by column chromatography over silica gel using 25% ethyl acetate in hexane and the desired compound Example 23 was isolated as light brown oil (50 mg, 48% yield, 99.51% purity). GCMS: m/z found 369.0 (CHE 300-METHOD); 1H NMR (400 MHz, DMSO-d6): δ 7.61 (d, J=4.8 Hz, 1H), 7.41 (dd, J=5.3, 8.6 Hz, 2H), 7.36-7.20 (m, 3H), 6.92-6.54 (m, 1H), 5.40-5.27 (m, 1H), 2.79 (s, 3H).

### Example 24 and Example 25

### N,N-dimethyl-5-(N-(2,2,2-trifluoro-1-(4-fluorophenyl)ethyl)sulfamoyl)thiophene-2-carboxamide (Example 24) and 5-(N-ethyl-N-(2,2,2-trifluoro-1-(4-fluorophenyl)ethyl)sulfamoyl)-N,N-dimethylthiophene-2-carboxamide (Example 25)

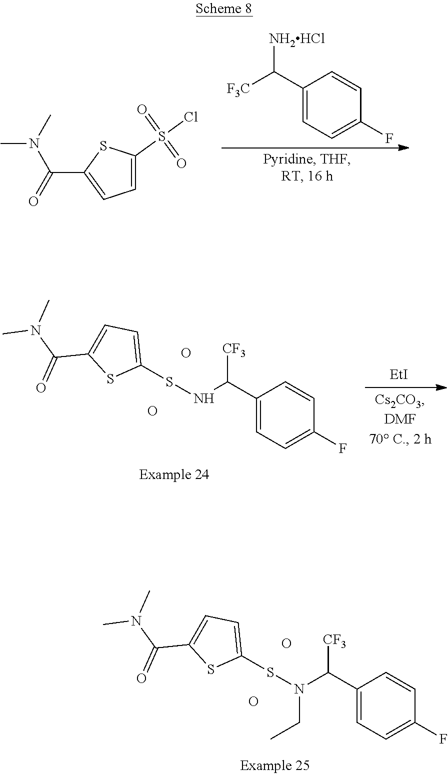

Desired compound Example 24 was synthesized from 5-(dimethylcarbamoyl)thiophene-2-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a white solid (150 mg, 33% yield, 95.06% purity). ¹H NMR (400 MHz, DMSO-d₆) δ 9.69 (d, J=9.9 Hz, 1H), 7.52 (dd, J=5.4, 8.5 Hz, 2H), 7.35 (d, J=3.9 Hz, 1H), 7.23 (d, J=4.0 Hz, 1H), 7.11 (t, J=8.7 Hz, 2H), 5.49-5.40 (m, 1H), 2.98 (s, 6H).

### 5-(N-ethyl-N-(2,2,2-trifluoro-1-(4-fluorophenyl)ethyl)sulfamoyl)-N,N-dimethylthiophene-2-carboxamide (Example 25

The ethylation of intermediate Example 24 was performed following the protocol as described in Method D at 70° C. The crude was purified by column chromatography over silica gel using 25% ethyl acetate in hexane and the desired compound Example 25 was isolated as a light-yellow sticky gum (70 mg, 52% yield, 98.7% purity). LCMS: m/z found 439.0[M+H]⁺, rt=3.48 min (Method 4) [Xbridge C18 column (5 μm, 50×4.6 mm)]; ¹H NMR (400 MHz, DMSO-d₆) δ 7.76 (d, J=4.0 Hz, 1H), 7.50 (d, J=4.0 Hz, 1H), 7.42 (dd, J=5.2, 8.5 Hz, 2H), 7.23 (t, J=8.9 Hz, 2H), 5.99 (q, J=8.8 Hz, 1H), 3.42-3.33 (m, 1H), 3.30-3.19 (m, 1H), 3.21-2.95 (m, 6H), 1.02 (t, J=7.0 Hz, 3H).

### Example 26

### 5-cyano-N-ethyl-N-(2,2,2-trifluoro-1-(4-fluorophenyl)ethyl)pyridine-3-sulfonamide

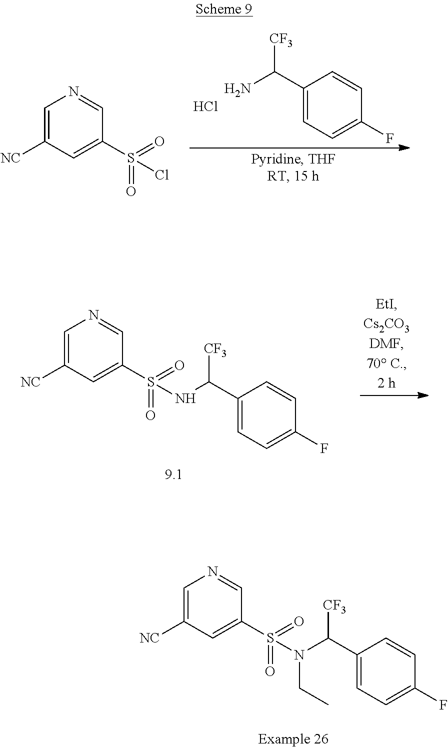 [SMILES: N#Cc1cncc(S(=O)(=O)Cl)c1 ; Cl ; NC(c1ccc(F)cc1)C(F)(F)F ; N#Cc1cncc(S(=O)(=O)NC(c2ccc(F)cc2)C(F)(F)F)c1 ; "EtI,Cs2CO3DMF,70° C.,2 h" ; CCN(C(c1ccc(F)cc1)C(F)(F)F)S(=O)(=O)c1cncc(C#N)c1]

### 5-cyano-N-(2,2,2-trifluoro-1-(4-fluorophenyl)ethyl)pyridine-3-sulfonamide

Intermediate 9.1 was synthesized from 5-cyanopyridine-3-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a colourless oil (300 mg, 34% yield). ¹H NMR (400 MHz, DMSO-d₆): δ 9.96-9.65 (m, 1H), 9.13 (d, J=1.5 Hz, 1H), 9.00 (d, J=2.0 Hz, 1H), 8.43 (t, J=2.1 Hz, 1H), 7.44 (dd, J=5.3, 8.6 Hz, 2H), 7.11 (t, J=8.8 Hz, 2H), 5.50 (d, J=7.7 Hz, 1H).

Ethylation of intermediate 9.1 was performed following the protocol as described in Method B at 70° C. The crude was purified by column chromatography over silica gel using 25% ethyl acetate in hexane and the desired compound Example 26 was isolated as a colourless gum (25 mg, 28% yield, 97.40% purity). LCMS: m/z found 388.1[M+H]⁺, rt=3.62 min (Method 4) [Xbridge C18 column (5 μm, 50×4.6 mm)]; ¹H NMR (400 MHz, DMSO-d₆) δ 9.31 (dd, J=1.9, 9.8 Hz, 2H), 8.90 (t, J=1.8 Hz, 1H), 7.45 (dd, J=5.3, 8.6 Hz, 2H), 7.26 (t, J=8.8 Hz, 2H), 6.07 (q, J=8.8 Hz, 1H), 3.56-3.32 (m, 2H), 0.99 (t, J=7.0 Hz, 3H).

### Example 27

### N-ethyl-6-methyl-N-(2,2,2-trifluoro-1-(4-fluorophenyl)ethyl)pyridine-2-sulfonamide

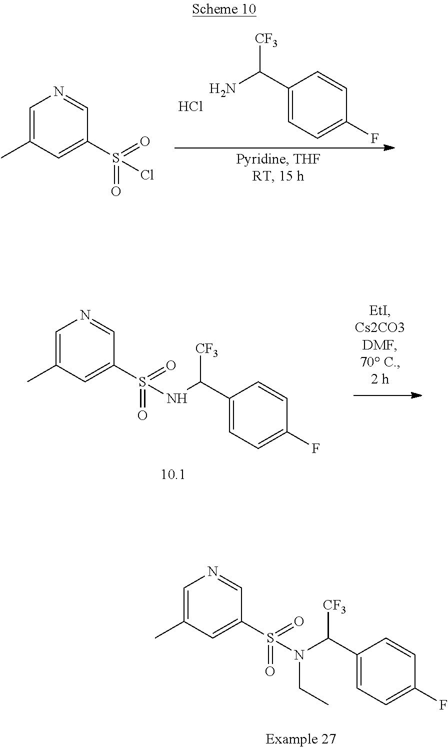 [SMILES: Cc1cncc(S(=O)(=O)Cl)c1 ; Cl ; NC(c1ccc(F)cc1)C(F)(F)F ; Cc1cncc(S(=O)(=O)NC(c2ccc(F)cc2)C(F)(F)F)c1 ; "EtI,Cs2CO3DMF,70° C.,2 h" ; CCN(C(c1ccc(F)cc1)C(F)(F)F)S(=O)(=O)c1cncc(C)c1]

6-methyl-N-(2,2,2-trifluoro-1-(4-fluorophenyl)ethyl)pyridine-2-sulfonamide 10.1: Intermediate 10.1 was synthesized from 6-methylpyridine-2-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a colourless oil (300 mg, 34% yield). ¹H NMR (400 MHz, DMSO-d₆) δ 9.41 (d, J=9.8 Hz, 1H), 7.80 (t, J=7.8 Hz, 1H), 7.64 (d, J=7.8 Hz, 1H), 7.47 (dd, J=5.3, 8.4 Hz, 2H), 7.33 (d, J=7.3 Hz, 1H), 7.10 (t, J=8.8 Hz, 2H), 5.45-5.15 (m, 1H), 2.27 (s, 3H).

Example 27: The ethylation of intermediate 6.1 was preformed following the protocol as described in Method D at 70° C. The crude was purified by column chromatography over silica gel using 25% ethyl acetate in hexane and the desired compound Example 27 was isolated as an off-white solid (10 mg, 23% yield, 99.70% purity). LCMS: m/z found 377.31[M+H]⁺, rt 3.20 min (Method 2) [Acquity BEH C8 column (1.7 μm, 50×2.1 mm)]; ¹H NMR (400 MHz, DMSO-d₆) δ 7.93 (t, J=8.0 Hz, 1H), 7.79 (d, J=7.6 Hz, 1H), 7.52 (t, J=8.8 Hz, 3H), 7.21 (t, J=8.8 Hz, 2H), 5.80 (q, J=9.2 Hz, 1H), 3.44-3.29 (m, 2H), 0.94 (t, J=6.8 Hz, 3H).

### Example 28

### N-ethyl-N-(2,2,2-trifluoro-1-(4-fluorophenyl)ethyl)-2,3-dihydrobenzo[b]thiophene-6-sulfonamide 1,1-dioxide

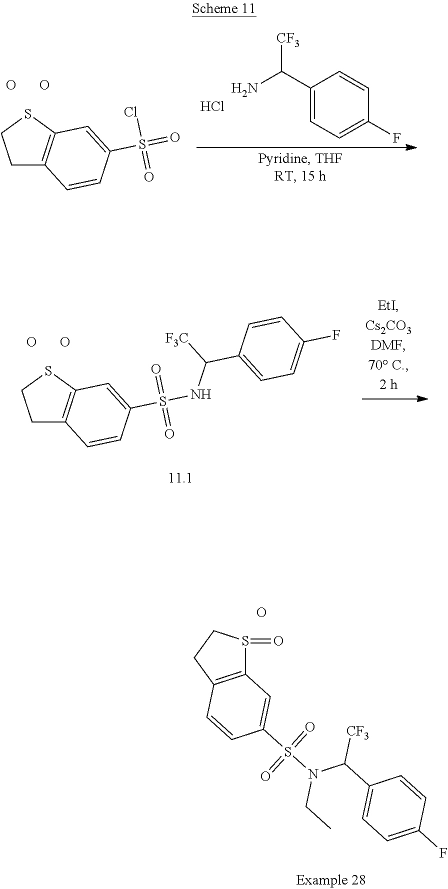

### N-(2,2,2-trifluoro-1-(4-fluorophenyl)ethyl)-2,3-dihydrobenzo[b]thiophene-6-sulfonamide 1,1-dioxide 11.1

Intermediate 11.1 was synthesized from 2,3-dihydrobenzo[b]thiophene-6-sulfonyl chloride 1,1-dioxide following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a brown oil (40 mg, 50% yield). ¹H NMR (400 MHz, DMSO-d₆) δ 9.48 (d, J=10.2 Hz, 1H), 7.92 (s, 1H), 7.88 (d, J=8.2 Hz, 1H), 7.59 (d, J=8.0 Hz, 1H), 7.43 (t, J=5.1 Hz, 2H), 7.06 (t, J=8.5 Hz, 2H), 5.59-5.40 (m, 1H), 3.62 (t, J=7.0 Hz, 2H), 3.36 (d, J=6.8 Hz, 2H).

Ethylation of intermediate 11.1 was performed following the protocol as described in Method B at 70° C. The crude was purified by column chromatography over silica gel using 25% ethyl acetate in hexane and the desired compound Example 28 was isolated as an off-white solid (50 mg, 47% yield, 99.44% purity). LCMS: m/z found ¹H NMR (400 MHz, DMSO-d₆) δ 8.32 (s, 1H), 8.17 (d, J=7.8 Hz, 1H), 7.76 (d, J=8.2 Hz, 1H), 7.52-7.44 (m, 2H), 7.21 (t, J=8.7 Hz, 2H), 6.27-6.05 (m, 1H), 3.70 (t, J=7.0 Hz, 2H), 3.46 (t, J=6.8 Hz, 2H), 3.29-3.17 (m, 2H), 0.93 (t, J=7.0 Hz, 3H).

### Example 29

### N-ethyl-N-(2,2,2-trifluoro-1-(4-fluorophenyl)ethyl)isochromane-7-sulfonamide

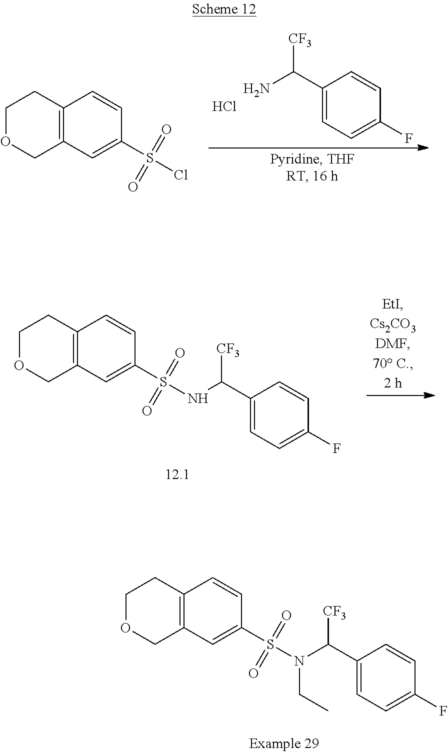 [SMILES: O=S(=O)(Cl)c1ccc2c(c1)COCC2 ; Cl ; NC(c1ccc(F)cc1)C(F)(F)F ; O=S(=O)(NC(c1ccc(F)cc1)C(F)(F)F)c1ccc2c(c1)COCC2 ; "EtI,Cs2CO3DMF,70° C.,2 h" ; CCN(C(c1ccc(F)cc1)C(F)(F)F)S(=O)(=O)c1ccc2c(c1)COCC2]

### N-(2,2,2-trifluoro-1-(4-fluorophenyl)ethyl)isochromane-7-sulfonamide 12.1

Intermediate 12.1 was synthesized from isochromane-7-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a colourless oil (370 mg, 74% yield). ¹H NMR (400 MHz, DMSO-d₆) δ 9.15 (d, J=10.0 Hz, 1H), 7.44-7.36 (m, 3H), 7.23 (s, 1H), 7.13 (d, J=8.0 Hz, 1H), 7.06 (t, J=8.8 Hz, 2H), 5.28 (t, J=8.0 Hz, 1H), 4.54 (s, 2H), 3.86-3.72 (m, 2H), 2.72 (t, J=5.4 Hz, 2H).

Ethylation of intermediate 12.1 was preformed following the protocol as described in Method D at 70° C. The crude was purified by column chromatography over silica gel using 25% ethyl acetate in hexane and the desired compound Example 29 was isolated as a colourless gum (50 mg, 47% yield, 99.63% purity). LCMS: m/z found 418.25[M+H]⁺, rt=3.27 min (Method 2) [Acquity BEH C8 column (1.7 μm, 50×2.1 mm)]; ¹H NMR (400 MHz, DMSO-d₆) δ 7.62 (d, J=8.0 Hz, 1H), 7.54 (s, 1H), 7.43 (t, J=5.2 Hz, 2H), 7.34 (d, J=8.0 Hz, 1H), 7.20 (t, J=8.4 Hz, 2H), 5.84 (q, J=8.8 Hz, 1H), 4.71 (s, 2H), 3.91 (t, J=5.6 Hz, 2H), 3.38-3.15 (m, 2H), 2.89 (t, J=5.6 Hz, 2H), 0.97 (t, J=7.1 Hz, 3H).

### Example 30

### 6-cyano-N-ethyl-N-(2,2,2-trifluoro-1-(4-fluorophenyl)ethyl)pyridine-2-sulfonamide

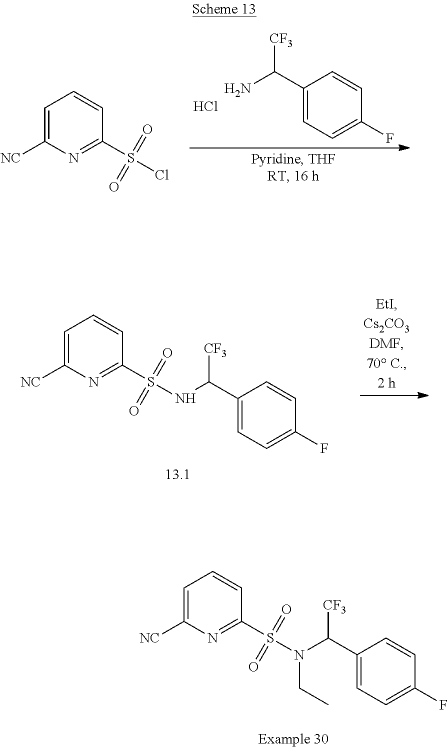 [SMILES: N#Cc1cccc(S(=O)(=O)Cl)n1 ; Cl ; NC(c1ccc(F)cc1)C(F)(F)F ; N#Cc1cccc(S(=O)(=O)NC(c2ccc(F)cc2)C(F)(F)F)n1 ; "EtI,Cs2CO3DMF,70° C.,2 h" ; CCN(C(c1ccc(F)cc1)C(F)(F)F)S(=O)(=O)c1cccc(C#N)n1]

### 6-cyano-N-(2,2,2-trifluoro-1-(4-fluorophenyl)ethyl)pyridine-2-sulfonamide

Intermediate 13.1 was synthesized from 6-cyanopyridine-2-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a colourless oil (310 mg, 34% yield). ¹H NMR (400 MHz, DMSO-d₆) δ 9.82 (d, J=9.9 Hz, 1H), 8.27-8.19 (m, 1H), 8.18-8.10 (m, 2H), 7.46 (dd, J=5.4, 8.6 Hz, 2H), 7.10 (t, J=8.8 Hz, 2H), 5.37 (q, J=8.5 Hz, 1H).

Ethylation of intermediate 13.1 was preformed following the protocol as described in Method D at 70° C. The crude was purified by column chromatography over silica gel using 25% ethyl acetate in hexane and the desired compound Example 30 was isolated as a colourless gum (25 mg, 58% yield, 96.19% purity). LCMS: m/z found 388.2[M+H]⁺, rt 3.12 min (Method 2) [Acquity BEH C18 column (1.7 μm, 50×2.1 mm)]; ¹H NMR (400 MHz, DMSO-d₆) δ 8.41-8.26 (m, 3H), 7.57-7.47 (m, 2H), 7.28-7.17 (m, 2H), 7.27-7.19 (m, 2H), 5.94 (q, J=8.7 Hz, 1H), 3.40 (q, J=7.0 Hz, 2H), 0.91 (t, J=7.0 Hz, 3H).

### Example 31

### 5-chloro-N-ethyl-4-methyl-N-(2,2,2-trifluoro-1-(4-fluorophenyl)ethyl)thiophene-2-sulfonamide

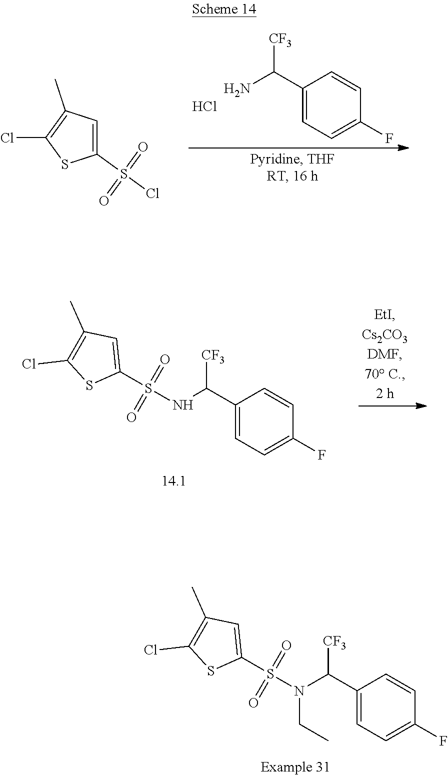 [SMILES: Cc1cc(S(=O)(=O)Cl)sc1Cl ; Cl ; NC(c1ccc(F)cc1)C(F)(F)F ; Cc1cc(S(=O)(=O)NC(c2ccc(F)cc2)C(F)(F)F)sc1Cl ; "EtI,Cs2CO3DMF,70° C.,2 h" ; CCN(C(c1ccc(F)cc1)C(F)(F)F)S(=O)(=O)c1cc(C)c(Cl)s1]

### 5-chloro-4-methyl-N-(2,2,2-trifluoro-1-(4-fluorophenyl)ethyl)thiophene-2-sulfonamide 14.1

Intermediate 14.1 was synthesized from 5-chloro-4-methylthiophene-2-sulfonyl chloride following the protocol as described in Method A using 2,2,2-trifluoro-1-(4-fluorophenyl)ethan-1-amine hydrochloride. Pyridine was used as base and THF as solvent. The compound was purified by column chromatography over silica gel using 25% ethyl acetate in hexane and isolated as a colourless oil (360 mg, 71% yield). ¹H NMR (400 MHz, DMSO-d₆) δ 9.62 (s, 1H), 7.49 (dd, J=5.5, 8.5 Hz, 2H), 7.13 (t, J=8.8 Hz, 3H), 5.31 (s, 1H), 1.98 (s, 3H).

Ethylation of intermediate 14.1 was preformed following the protocol as described in Method D at 70° C. The crude was purified by column chromatography over silica gel using 25% ethyl acetate in hexane and the desired compound Example 31 was isolated as a colourless gum (50 mg, 47% yield, 98.18% purity). LCMS: m/z found 415.9[M+H]⁺, rt=4.06 min (Method 4) [Xbridge C18 column (5 μm, 50×4.6 mm)]; ¹H NMR (400 MHz, DMSO-d₆) δ 7.63 (s, 1H), 7.45 (dd, J=5.3, 8.6 Hz, 2H), 7.27 (t, J=8.8 Hz, 2H), 5.92 (q, J=8.7 Hz, 1H), 3.39-3.32 (m, 1H), 3.27-3.19 (m, 1H), 2.16 (s, 3H), 1.01 (t, J=7.0 Hz, 3H).

### Example 32 and Example 33

### (S)—N-ethyl-6-methyl-N-(2,2,2-trifluoro-1-(4-fluorophenyl)ethyl)pyridine-2-sulfonamide (Example 32) and (R)—N-ethyl-6-methyl-N-(2,2,2-trifluoro-1-(4-fluorophenyl)ethyl)pyridine-2-sulfonamide (Example 33)

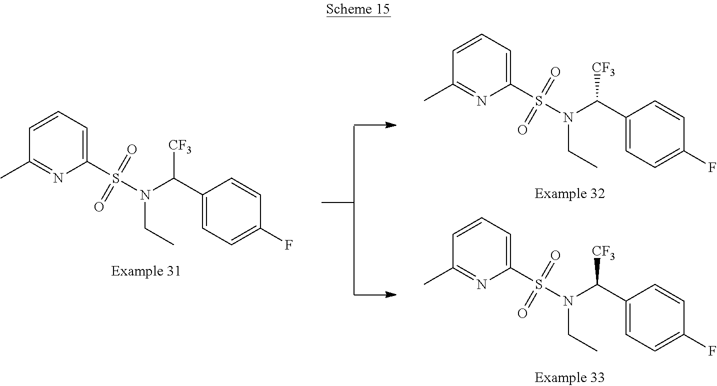 [SMILES: CCN(C(c1ccc(F)cc1)C(F)(F)F)S(=O)(=O)c1cccc(C)n1 ; CCN([C@@H](c1ccc(F)cc1)C(F)(F)F)S(=O)(=O)c1cccc(C)n1 ; CCN([C@H](c1ccc(F)cc1)C(F)(F)F)S(=O)(=O)c1cccc(C)n1]

Chiral separation of racemic Example 30 provided both enantiomers as mentioned below. Chiral separation Method: Chiral separation was done on Agilent 1200 series instrument. Column name: Chiralcel OJ-H (250×21 mm), 5μ, operating at ambient temperature and flow rate of 21.0 mL/min. Mobile phase: the mixture of 85% Hexane and 15% Ethanol, held this isocratic mixture up to 20 min with wavelength of 268 nm.

Example 32: 10 mg (Colorless Sticky Gum, 99.84% purity). LCMS: m/z found 377.1[M+H]⁺, rt 3.71 min (Method 4) [Xbridge C18 column (5 μm, 50×4.6 mm)]; ¹H NMR (400 MHz, Chloroform-d) δ 7.81 (d, J=7.7 Hz, 1H), 7.74 (t, J=7.7 Hz, 1H), 7.57 (dd, J=5.2, 8.6 Hz, 2H), 7.30 (d, J=7.6 Hz, 1H), 7.04 (t, J=8.6 Hz, 2H), 5.84 (q, J=8.4 Hz, 1H), 3.45-3.27 (m, 2H), 2.60 (s, 3H), 0.93 (t, J=6.8 Hz, 3H).

Example 33: 10 mg (Light yellow Sticky Gum, 99.27% purity). LCMS: m/z found 377.1[M+H]⁺, rt 3.73 min (Method 4) [Xbridge C18 column (5 μm, 50×4.6 mm)]; ¹H NMR (400 MHz, Chloroform-d) δ 7.80 (d, J=7.7 Hz, 1H), 7.74 (t, J=7.6 Hz, 1H), 7.57 (dd, J=5.2, 8.5 Hz, 2H), 7.30 (d, J=7.7 Hz, 1H), 7.04 (t, J=8.6 Hz, 2H), 5.84 (q, J=8.2 Hz, 1H), 3.41-3.31 (m, 2H), 2.60 (s, 3H), 0.94 (t, J=7.2 Hz, 3H).

### Example 34 and Example 35

### (S)-5-cyano-N-ethyl-N-(2,2,2-trifluoro-1-(4-fluorophenyl)ethyl)pyridine-3-sulfonamide (Example 34) and (R)-5-cyano-N-ethyl-N-(2,2,2-trifluoro-1-(4-fluorophenyl)ethyl)pyridine-3-sulfonamide (Example 35)

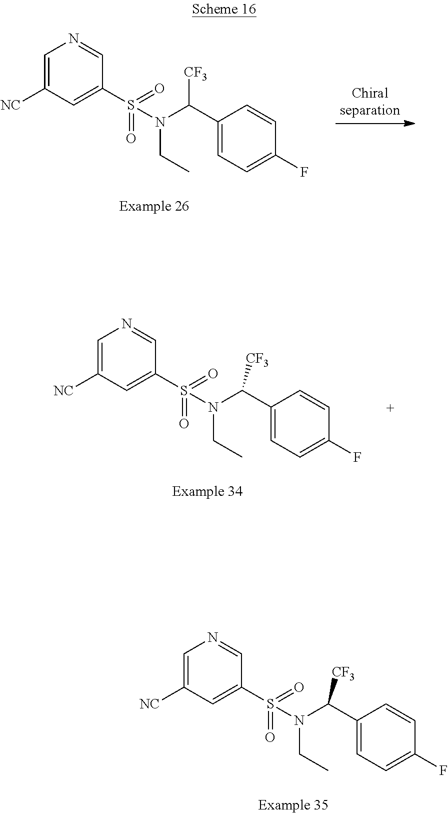 [SMILES: CCN(C(c1ccc(F)cc1)C(F)(F)F)S(=O)(=O)c1cncc(C#N)c1 ; CCN([C@@H](c1ccc(F)cc1)C(F)(F)F)S(=O)(=O)c1cncc(C#N)c1 ; CCN([C@H](c1ccc(F)cc1)C(F)(F)F)S(=O)(=O)c1cncc(C#N)c1]

Chiral separation of racemic Example 26 provided both enantiomers as shown in Scheme 16. Chiral separation method: chiral separation was done on Agilent 1200 series instrument. Column name: Chiralpak IG (250×21 mm), 5μ, operating at ambient temperature and flow rate of 21.0 mL/min. Mobile phase: the mixture of 80% hexane, 10% dichloromethane and 10% ethanol, held this isocratic mixture up to 18 min with wavelength of 230 nm.

Example 34: Yellow Sticky Gum (10 mg, 99.88% purity). LCMS: m/z found 386.1[M+H]⁺, rt=3.57 min (Method 4) [Xbridge C18 column (5 μm, 50×4.6 mm)]; ¹H NMR (400 MHz, DMSO-d₆) δ 9.31 (dd, J=1.9, 9.8 Hz, 2H), 8.90 (t, J=1.8 Hz, 1H), 7.45 (dd, J=5.3, 8.6 Hz, 2H), 7.26 (t, J=8.8 Hz, 2H), 6.07 (q, J=8.8 Hz, 1H), 3.52-3.32 (m, 2H), 0.99 (t, J=7.0 Hz, 3H).

Example 35: Yellow Sticky Gum (10 mg, 94.36%% purity). LCMS: m/z found 386.1[M+H]⁺, rt=3.60 min (Method 4) [Xbridge C18 column (5 μm, 50×4.6 mm)]; ¹H NMR (400 MHz, DMSO-d₆) δ 9.31 (dd, J=2.1, 9.4 Hz, 2H), 8.89 (t, J=2.1 Hz, 1H), 7.45 (dd, J=5.3, 8.6 Hz, 2H), 7.26 (t, J=8.8 Hz, 2H), 6.08 (q, J=8.8 Hz, 1H), 3.56-3.31 (m, 2H), 1.00 (t, J=7.2 Hz, 3H).

### Example 36: N-ethyl-N-(2,2,2-trifluoro-1-(4-fluorophenyl)ethyl)-1,3-dihydroisobenzofuran-5-sulfonamide

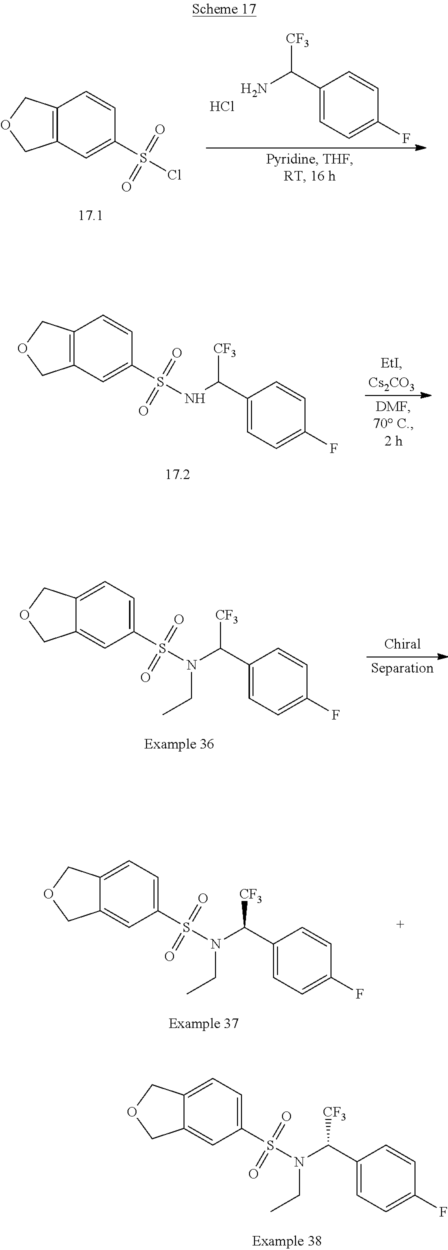

N-(2,2,2-trifluoro-1-(4-fluorophenyl)ethyl)-1,3-dihydroisobenzofuran-5-sulfonamide 15.2: Method O: To a stirred solution of dihydroisobenzofuran-5-sulfonyl chloride 17.1 (250.0 mg, 1.14 mmol) and 2,2,2-trifluoro-1-(4-fluorophenyl)ethan-1-amine hydrochloride (262 mg, 1.14 mmol) in THF (1 mL) was added pyridine (0.7 ml, 8.01 mmol). Reaction mixture was stirred at RT for overnight. The volatiles were evaporated under reduced pressure and the residue was purified by column chromatography (100-200 mesh silica gal, eluent 30% EtOAC in hexane) to afford N-(2,2,2-trifluoro-1-(4-fluorophenyl)ethyl)-1,3-dihydroisobenzofuran-5-sulfonamide 17.2 as colourless oil (220 mg, 47% yield). ¹H NMR (400 MHz, DMSO-d₆): δ 9.23 (d, J=10.4 Hz, 1H), 7.59-7.49 (m, 2H), 7.41 (t, J=5.7 Hz, 2H), 7.31 (d, J=7.8 Hz, 1H), 7.07 (t, J=8.8 Hz, 2H), 5.43-5.19 (m, 1H), 4.92 (d, J=18.2 Hz, 4H)

N-ethyl-N-(2,2,2-trifluoro-1-(4-fluorophenyl)ethyl)-1,3-dihydroisobenzofuran-5-sulfonamide: Method D: To a stirred solution of compound N-(2,2,2-trifluoro-1-(4-fluorophenyl)ethyl)-1,3-dihydroisobenzofuran-5-sulfonamide 17.2 (220.0 mg 0.58 mmol) in DMF (0.5 mL) was added Cs₂CO₃ (286 mg, 0.88 mmol) and ethyl iodide (0.07 mL, 0.88 mmol). Reaction mixture was stirred 70° C. for 2 h. Reaction mixture was diluted with water (5 mL) and extracted with EtOAc (2×10 mL). The combined organic layer was washed with water (10 mL) and brine (10 mL); dried over anhydrous sodium sulfate and concentrated under reduced pressure. The crude was purified by combi-flash column to afford N-ethyl-N-(2,2,2-trifluoro-1-(4-fluorophenyl)ethyl)-1,3-dihydroisobenzofuran-5-sulfonamide as off white solid (80 mg, 33% yield, 99.59% purity). LCMS: m/z found 404.2 [M+H]⁺, rt=3.54 min (Method 4) [Waters Xbridge C18 column (5 μm, 50×4.6 mm)]: ¹H NMR (400 MHz, Chloroform-d): δ 7.80 (d, J=8.0 Hz, 1H), 7.73 (s, 1H), 7.46 (dd, J=5.2, 8.6 Hz, 2H), 7.37 (d, J=8.0 Hz, 1H), 7.08 (t, J=8.6 Hz, 2H), 5.83 (q, J=8.4 Hz, 1H), 5.34-4.95 (m, 4H), 3.40-3.26 (m, 1H), 3.19-3.04 (m, 1H), 0.90 (t, J=7.0 Hz, 3H).

### Example 37 and Example 38

### (S)—N-ethyl-N-(2,2,2-trifluoro-1-(4-fluorophenyl)ethyl)-1,3-dihydroisobenzofuran-5-sulfonamide (Example 37 (EN-1)) and (R)—N-ethyl-N-(2,2,2-trifluoro-1-(4-fluorophenyl)ethyl)-1,3-dihydroisobenzofuran-5-sulfonamide (Example 38 (EN-2

Chiral separation of racemic Example 36 provided both the enantiomers as mentioned below. Chiral separation method: Chiral separation was done on Agilent 1200 series instrument. Column: CHIRALCEL OJ-H (250×21 mm), 5μ, operating at ambient temperature and flow rate of 21.0 mL/min. Mobile phase was 0.1% of Isopropylamine in the mixture of 90% Hexane and 10% of Ethanol, held this isocratic mixture run up to 24 min with wavelength of 250 nm.

Example 37 [EN-1]: Off white solid (96.80% purity). LCMS: m/z found 404.2 [M+H]⁺, rt=3.62 min (Method 4) [Waters Xbridge C18 column (5 μm, 50×4.6 mm)]: ¹H NMR (400 MHz, Chloroform-d): δ 7.80 (d, J=8.4 Hz, 1H), 7.73 (s, 1H), 7.46 (t, J=5.6 Hz, 2H), 7.36 (d, J=8.0 Hz, 1H), 7.08 (t, J=8.6 Hz, 2H), 5.82 (q, J=8.5 Hz, 1H), 5.15 (d, J=4.0 Hz, 4H), 3.38-3.28 (m, 1H), 3.17-3.07 (m, 1H), 0.91 (t, J=7.1 Hz, 3H).

Example 38 [EN-2]: Off white solid (98.40% purity). LCMS: m/z found 404.2 [M+H]⁺, rt=3.74 min (Method 4) [Waters Xbridge C18 column (5 μm, 50×4.6 mm)]: ¹H NMR (400 MHz, DMSO-d₆): δ 7.88-7.79 (m, 2H), 7.51 (d, J=7.8 Hz, 1H), 7.42 (dd, J=5.4, 8.6 Hz, 2H), 7.23 (t, J=8.8 Hz, 2H), 5.95 (q, J=8.7 Hz, 1H), 5.05 (d, J=9.4 Hz, 4H), 0.93 (t, J=7.0 Hz, 3H).

### Example 39

### N-ethyl-N-(2,2,2-trifluoro-1-(4-fluorophenyl)ethyl)-2,3-dihydrobenzofuran-6-sulfonamide

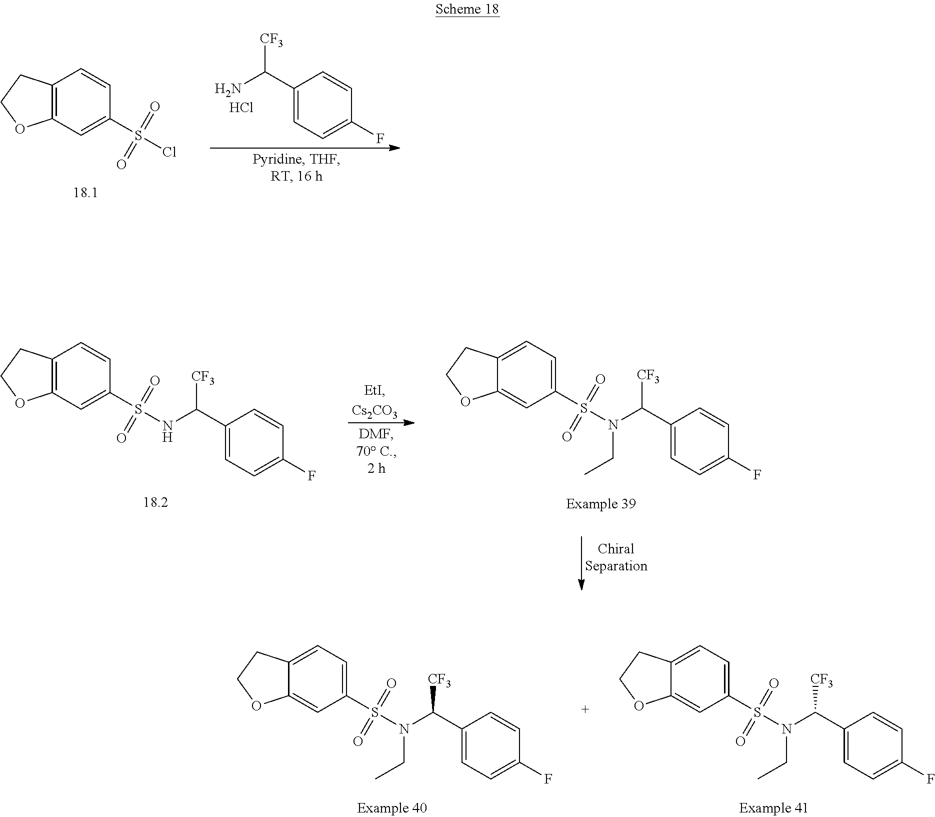

N-(2,2,2-trifluoro-1-(4-fluorophenyl)ethyl)-2,3-dihydrobenzofuran-6-sulfonamide 18.2: Intermediate 18.2 was synthesized from 2,3-dihydrobenzofuran-6-sulfonyl chloride 18.1 following the protocol as described in Method O using 2,2,2-trifluoro-1-(4-fluorophenyl)ethan-1-amine hydrochloride. The crude was purified by combi-flash column to afford N-(2,2,2-trifluoro-1-(4-fluorophenyl)ethyl)-2,3-dihydrobenzofuran-6-sulfonamide as colourless oil (200 mg, 43% yield). ¹H NMR (400 MHz, DMSO-d₆): δ 9.14 (d, J=10.4 Hz, 1H), 7.43 (dd, J=5.5, 8.6 Hz, 2H), 7.21 (d, J=7.7 Hz, 1H), 7.16-7.03 (m, 3H), 6.91 (d, J=1.6 Hz, 1H), 5.38-5.21 (m, 1H), 4.51 (t, J=8.8 Hz, 2H), 3.13 (t, J=8.8 Hz, 2H).

N-ethyl-N-(2,2,2-trifluoro-1-(4-fluorophenyl)ethyl)-2,3-dihydrobenzofuran-6-sulfonamide Example 39: The ethylation of 18.2 was preformed following the protocol as described in Method D using Cs₂CO₃. The crude was purified by combiflash column chromatography to afford N-ethyl-N-(2,2,2-trifluoro-1-(4-fluorophenyl)ethyl)-2,3-dihydrobenzofuran-6-sulfonamide as off white solid (70 mg, 32% purity, 99.25% purity). LCMS: m/z found 404.2 [M+H]⁺, rt=3.52 min (Method 4) [Waters Xbridge C18 column (5 μm, 50×4.6 mm)]; ¹H NMR (400 MHz, Chloroform-d) δ 7.48-7.34 (m, 3H), 7.32-7.20 (m, 2H), 7.06 (t, J=8.6 Hz, 2H), 5.79 (q, J=8.6 Hz, 1H), 4.65 (t, J=8.9 Hz, 2H), 3.38-3.23 (m, 3H), 3.16-3.02 (m, 1H), 0.92 (t, J=7.1 Hz, 3H).

### Example 40 and Example 41

### (R)—N-ethyl-N-(2,2,2-trifluoro-1-(4-fluorophenyl)ethyl)-2,3-dihydrobenzofuran-6-sulfonamide Example 40 [EN-1] and (S)—N-ethyl-N-(2,2,2-trifluoro-1-(4-fluorophenyl)ethyl)-2,3-dihydrobenzofuran-6-sulfonamide Example 41 [EN-2]

Chiral separation of racemic Example 39 provided both the enantiomers as mentioned below. Chiral separation method: Chiral separation was done on Agilent 1200 series instrument. Column: CHIRALPAK IG (250×21 mm), 5μ, operating at ambient temperature and flow rate of 21.0 mL/min. Mobile phase was a mixture of 95% Hexane and 5% of Isopropylalcohol, held this isocratic mixture run up to 35 min with wavelength of 228 nm.

Example 40: Off white solid (99.81% purity). LCMS: m/z found 404.2 [M+H]⁺, rt=3.76 min (Method 4) [Waters Xbridge C18 column (5 μm, 50×4.6 mm)]; ¹H NMR (400 MHz, Chloroform-d): δ 7.48-7.35 (m, 3H), 7.28 (d, J=7.5 Hz, 1H), 7.26-7.16 (m, 2H), 7.06 (t, J=8.4 Hz, 2H), 5.79 (q, J=8.4 Hz, 1H), 4.66 (t, J=8.8 Hz, 2H), 3.28 (t, J=8.6 Hz, 2H), 3.17-3.02 (m, 1H), 0.92 (t, J=7.0 Hz, 3H).

Example 4: Off white sticky gum (99.90% purity). LCMS: m/z found 404.2 [M+H]⁺, rt=3.74 min (Method 4) [Waters Xbridge C18 column (5 μm, 50×4.6 mm)]; ¹H NMR (400 MHz, DMSO-d₆) δ 7.46-7.35 (m, 4H), 7.30-7.18 (m, 3H), 5.95 (q, J=8.8 Hz, 1H), 4.61 (t, J=8.8 Hz, 2H), 3.36-3.20 (m, 4H), 3.19-3.05 (m, 1H), 0.90 (t, J=7.0 Hz, 3H).

### Example 42

### 5-Cyano-N-ethyl-N-(2,2,2-trifluoro-1-(4-fluorophenyl)ethyl)thiophene-3-sulfonamide

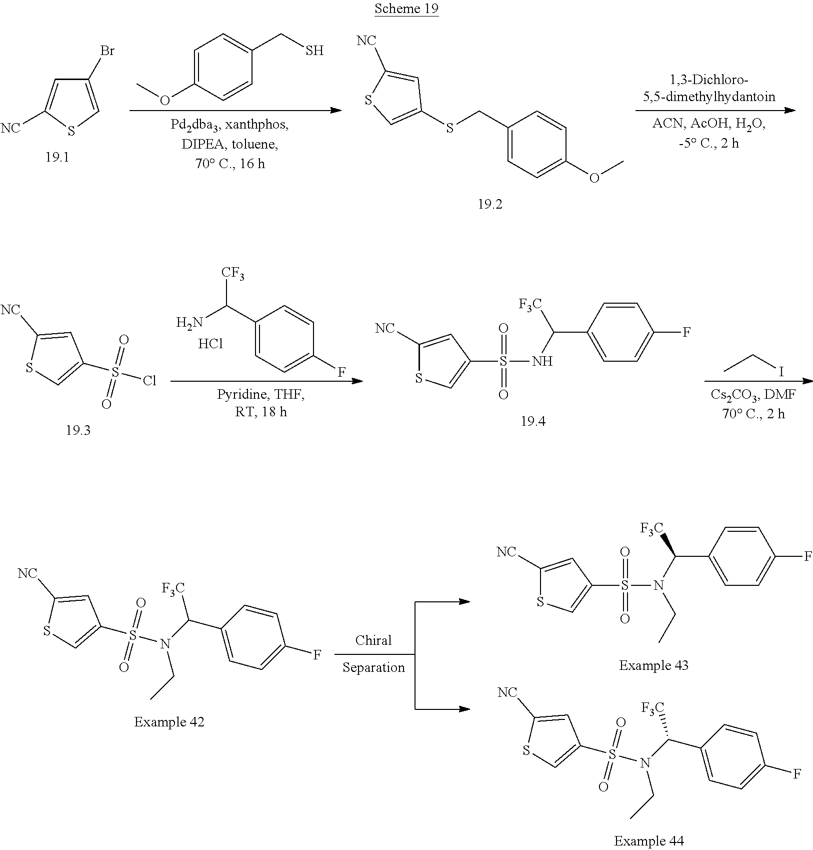

4-((4-Methoxybenzyl)thio)thiophene-2-carbonitrile 19.2: Intermediate 19.2 was synthesized from 4-bromothiophene-2-carbonitrile 19.1 following a method analogous to that described in Method I using (4-methoxyphenyl)methanethiol at 70° C. The crude was purified by combi-flash column using 0-30% ethyl acetate-hexane to afford 4-((4-methoxybenzyl)thio)thiophene-2-carbonitrile (600 mg, 86% yield) as yellow oil. ¹H NMR (400 MHz, DMSO-d₆): δ 7.94 (d, J=1.2 Hz, 1H), 7.80 (d, J=1.2 Hz, 1H), 7.22 (d, J=8.5 Hz, 2H), 6.86 (d, J=8.5 Hz, 2H), 4.17 (s, 2H), 3.71 (s, 3H).

5-Cyanothiophene-3-sulfonyl chloride 19.3. Method T: Intermediate 19.2 (227 mg, 0.906 mmol) was dissolved in CH₃CN (5 mL) and to the solution was added a mixture of water (0.3 mL) and AcOH (0.1 mL). It was then cooled at −5° C. and 1,3-dichloro-5,5-dimethyl hydantoin (268 mg, 1.359 mmol) was added to it. The RM was then stirred for 30 mins at −5° C. The RM was then quenched with water and extracted with DCM (10 mL). The organic part was washed with brine, dried and the solvent was evaporated under reduced pressure at RT. The crude was used in the forwarding step immediately without further purification. The crude sulfonyl chloride 19.3 was used immediately in the forwarding step without further purification.

5-Cyano-N-(2,2,2-trifluoro-1-(4-fluorophenyl)ethyl)thiophene-3-sulfonamide 19.4: Intermediate 19.4 was synthesized from sulfonyl chloride 19.3 following the protocol as described in Method O using 2,2,2-trifluoro-1-(4-fluorophenyl)ethan-1-amine hydrochloride. The compound was purified by combi-flash column using 30% EA-Hexane to afford 5-cyano-N-(2,2,2-trifluoro-1-(4-fluorophenyl)ethyl)thiophene-3-sulfonamide (100 mg, 38% yield) as light yellow gum. 1H NMR (400 MHz, DMSO-d₆) δ 9.54 (d, J=9.8 Hz, 1H), 8.37 (s, 1H), 7.82 (s, 1H), 7.46 (dd, J=5.4, 8.5 Hz, 2H), 7.14 (t, J=8.8 Hz, 2H), 5.44-5.35 (m, 1H).

5-Cyano-N-ethyl-N-(2,2,2-trifluoro-1-(4-fluorophenyl)ethyl)thiophene-3-sulfonamide (Example 42): The ethylation of intermediate 19.4 was preformed following the protocol as described in Method D at 70° C. The crude was purified by combi-flash column using 10% EA-Hexane and the compound 5-cyano-N-ethyl-N-(2,2,2-trifluoro-1-(4-fluorophenyl)ethyl)thiophene-3-sulfonamide (Example 42) was isolated as off white sticky gum (30 mg, 28% yield, 98.18% purity). LCMS: m/z found 390.9 [M−H], rt=3.70 min (Method 4) [Waters Xbridge C18 column (5 μm, 50×4.6 mm)]; ¹H NMR (400 MHz, Chloroform-d): δ 8.16 (d, J=1.3 Hz, 1H), 7.80 (d, J=1.2 Hz, 1H), 7.46 (dd, J=5.1, 8.6 Hz, 2H), 7.17-7.06 (m, 2H), 5.78 (q, J=8.4 Hz, 1H), 3.44-3.30 (m, 1H), 3.24-3.10 (m, 1H), 0.93 (t, J=7.0 Hz, 3H).

### Example 43 and Example 44

### 5-Cyano-N-ethyl-N-(2,2,2-trifluoro-1-(4-fluorophenyl)ethyl)thiophene-3-sulfonamide (Example 43 [EN-1]) and 5-cyano-N-ethyl-N-(2,2,2-trifluoro-1-(4-fluorophenyl)ethyl)thiophene-3-sulfonamide (Example 44 [EN-2]

Chiral separation of racemic Example 42 provided both the enantiomers as mentioned below. Chiral separation method: Chiral separation was done on Agilent 1200 series instrument. Column name: CHIRALCEL OJ-H (250×21 mm), 5μ, operating at ambient temperature and flow rate of 21.0 mL/min. Mobile phase was 0.1% isopropylamine in the mixture of 90% Hexane and 10% of Ethanol, held this isocratic mixture run up to 24 min with wavelength of 250 nm.

Example 43: Off white sticky gum (99.78% purity). LCMS: m/z found 391.2 [M−H]⁻, rt=9.61 min (Method 6) [Waters Xbridge C18 column (5 μm, 50×4.6 mm)]; ¹H NMR (400 MHz, Chloroform-d): δ 8.16 (d, J=0.8 Hz, 1H), 7.79 (s, 1H), 7.46 (dd, J=5.1, 8.5 Hz, 2H), 7.11 (t, J=8.6 Hz, 2H), 5.77 (q, J=8.4 Hz, 1H), 3.44-3.30 (m, 1H), 3.24-3.10 (m, 1H), 0.94 (t, J=7.1 Hz, 3H).

Example 44: Light brown sticky gum (99.44% purity). LCMS: m/z found 391.2 [M−H]⁻, rt=9.63 min (Method 6) [Waters Xbridge C18 column (5 μm, 50×4.6 mm)]; ¹H NMR (400 MHz, Chloroform-d): δ 8.16 (s, 1H), 7.80 (s, 1H), 7.46 (t, J=5.1 Hz, 2H), 7.11 (t, J=8.4 Hz, 2H), 5.79 (q, J=8.4 Hz, 1H), 3.46-3.29 (m, 1H), 3.24-3.10 (m, 1H), 0.94 (t, J=6.8 Hz, 3H).

### Example 45

### 5-Cyano-N-ethyl-3-methyl-N-(2,2,2-trifluoro-1-(4-fluorophenyl)ethyl)thiophene-2-sulfonamide

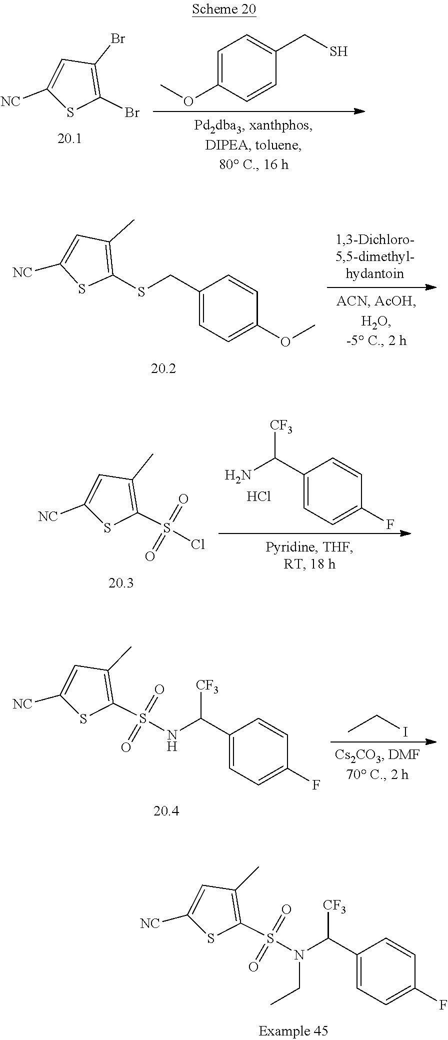

5-((4-Methoxybenzyl)thio)-4-methylthiophene-2-carbonitrile 20.2: Intermediate 20.2 was synthesized from 5-bromo-4-methylthiophene-2-carbonitrile 20.1 following a method analogous to that described in Method I using (4-methoxyphenyl)methanethiol at 80° C. The crude was purified by combi-flash column chromatography using 30% ethyl acetate-hexane to afford 5-((4-methoxybenzyl)thio)-4-methylthiophene-2-carbonitrile (250 mg, 74% yield) as yellow gum. ¹H NMR (400 MHz, DMSO-d₆): δ 7.85-7.75 (m, 1H), 7.11 (d, J=8.7 Hz, 2H), 6.85 (d, J=8.6 Hz, 2H), 4.07 (s, 2H), 3.72 (s, 3H), 2.03 (s, 3H).

5-cyano-3-methylthiophene-2-sulfonyl chloride 20.3: Sulfonyl chloride 20.3 was synthesized from Intermediate 20.2 following the procedure described in Method T. The crude sulfonyl chloride was used immediately in the forwarding step without further purification.

5-Cyano-3-methyl-N-(2,2,2-trifluoro-1-(4-fluorophenyl)ethyl)thiophene-2-sulfonamide 20.4: Intermediate 20.4 was synthesized from sulfonyl chloride 20.3 following the protocol as described in Method O using 2,2,2-trifluoro-1-(4-fluorophenyl)ethan-1-amine hydrochloride. The compound was purified by combi-flash column using 30% EA-Hexane to afford 5-cyano-3-methyl-N-(2,2,2-trifluoro-1-(4-fluorophenyl)ethyl)thiophene-2-sulfonamide as colourless gum (80 mg, 39% yield). LCMS: m/z found 377 [M−H]), rt=3.43 min (Method 4) Waters Xbridge C18 column (5 μm, 50×4.6 mm).

5-cyano-N-ethyl-3-methyl-N-(2,2,2-trifluoro-1-(4-fluorophenyl)ethyl)thiophene-2-sulfonamide: The ethylation of intermediate 20.4 was preformed following the protocol as described in Method D at 70° C. The crude was purified by combi-flash column using 15% EA-Hexane and the compound 5-cyano-N-ethyl-3-methyl-N-(2,2,2-trifluoro-1-(4-fluorophenyl)ethyl)thiophene-2-sulfonamide (Example 45) was isolated as off white sticky gum (20 mg, 34% yield, 99.98% purity). LCMS: m/z found 424.1 [M+H]⁺ 17, rt=3.70 min (Method 4) [Waters Xbridge C18 column (5 μm, 50×4.6 mm)]; ¹H NMR (400 MHz, Chloroform-d): δ 7.47 (dd, J=5.1, 8.4 Hz, 2H), 7.38 (s, 1H), 7.11 (t, J=8.6 Hz, 2H), 5.66 (q, J=8.3 Hz, 1H), 3.68-3.19 (m, 2H), 2.48 (s, 3H), 0.93 (t, J=7.1 Hz, 3H).

### Example 46

### 5-(N-ethyl-N-(2,2,2-trifluoro-1-(4-fluorophenyl)ethyl)sulfamoyl)thiazole-2-carboxamide

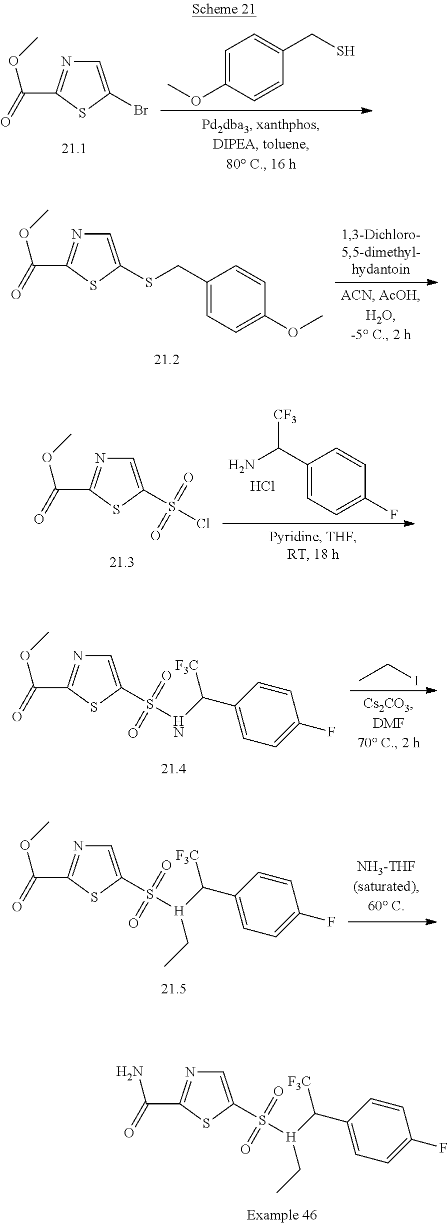

5-((4-Methoxybenzyl)thio)thiazole-2-carboxylate 21.2: Intermediate 21.2 was synthesized from methyl 5-bromothiazole-2-carboxylate 21.1 following a method analogous to that described in Method I using (4-methoxyphenyl)methanethiol at 80° C. The crude was purified by combi-flash column chromatography using 30% ethyl acetate-hexane to afford methyl 5-((4-methoxybenzyl)thio)thiazole-2-carboxylate (1 g, 94% yield) as yellow solid. ¹H NMR (400 MHz, DMSO-d₆): δ 7.92 (s, 1H), 7.21 (d, J=8.4 Hz, 2H), 6.87 (d, J=8.6 Hz, 2H), 4.22 (s, 2H), 3.88 (s, 3H), 3.72 (s, 3H).

Methyl 5-(chlorosulfonyl)thiazole-2-carboxylate 21.3: Sulfonyl chloride 21.3 was synthesized from Intermediate 21.2 following the procedure described in Method T. The crude sulfonyl chloride was purified by combi-flash column chromatography using 10% EA-Hexane to afford methyl 5-(chlorosulfonyl)thiazole-2-carboxylate (700 mg, 85% yield). ¹H NMR (400 MHz, DMSO-d₆): δ 7.99 (s, 1H), 3.90 (s, 3H).

Methyl 5-(N-(2,2,2-trifluoro-1-(4-fluorophenyl)ethyl)sulfamoyl)thiazole-2-carboxylate 21.4: Intermediate 21.4 was synthesized from sulfonyl chloride 21.3 following the protocol as described in Method O using 2,2,2-trifluoro-1-(4-fluorophenyl)ethan-1-amine hydrochloride. The compound was purified by combi-flash column using 50% EA-Hexane to afford methyl 5-(N-(2,2,2-trifluoro-1-(4-fluorophenyl)ethyl)sulfamoyl)thiazole-2-carboxylate (500 mg, 43% yield). ¹H NMR (400 MHz, DMSO-d₆): δ 10.14 (d, J=8.9 Hz, 1H), 8.29 (s, 1H), 7.52 (dd, J=5.3, 8.6 Hz, 2H), 7.16 (t, J=8.7 Hz, 2H), 5.67-5.47 (m, 1H), 3.91 (s, 3H).

Methyl 5-(N-ethyl-N-(2,2,2-trifluoro-1-(4-fluorophenyl)ethyl)sulfamoyl)thiazole-2-carboxylate 21.5: The ethylation of intermediate 21.4 was preformed following the protocol as described in Method D at 70° C. The crude was purified by combi-flash column using 20% EA-Hexane and the compound methyl 5-(N-ethyl-N-(2,2,2-trifluoro-1-(4-fluorophenyl)ethyl)sulfamoyl)thiazole-2-carboxylate was isolated as off white solid (120 mg, 70% yield). ¹H NMR (400 MHz, Chloroform-d): δ 8.34 (s, 1H), 7.44 (t, J=6.9 Hz, 3H), 7.11 (t, J=8.3 Hz, 2H), 5.90-5.66 (m, 1H), 4.04 (s, 3H), 3.45-3.34 (m, 1H), 3.32-3.20 (m, 1H), 1.01 (t, J=7.0 Hz, 3H).

5-(N-ethyl-N-(2,2,2-trifluoro-1-(4-fluorophenyl)ethyl)sulfamoyl)thiazole-2-carboxamide: Intermediate 21.5 (40 mg, 0.09 mmol) was heated with NH₃ in THF (1.5 mL) in a sealed tube at 70° C. for 12 h. The volatiles were evaporated under reduced pressure and crude was purified by combi-flash column chromatography using 50% EA-Hexane to afford 5-(N-ethyl-N-(2,2,2-trifluoro-1-(4-fluorophenyl)ethyl)sulfamoyl)thiazole-2-carboxamide (Example 46, 10 mg, 26% yield, 95.12% purity) as white solid. LCMS: m/z found 412 [M+H]⁺17, rt=2.79 min (Method 9) [Waters Xbridge C18 column (3.5 μm, 50×3 mm)]; ¹H NMR (400 MHz, DMSO-d₆): δ 8.57 (s, 1H), 8.49 (s, 1H), 8.19 (s, 1H), 7.47 (dd, J=5.3, 8.7 Hz, 2H), 7.27 (t, J=8.8 Hz, 2H), 6.06 (q, J=8.7 Hz, 1H), 3.49-3.32 (m, 2H), 1.03 (t, J=7.0 Hz, 3H).

### Example 47

### 5-(Azetidin-3-yl)-N-ethyl-N-(2,2,2-trifluoro-1-(4-fluorophenyl)ethyl)thiophene-2-sulfonamide

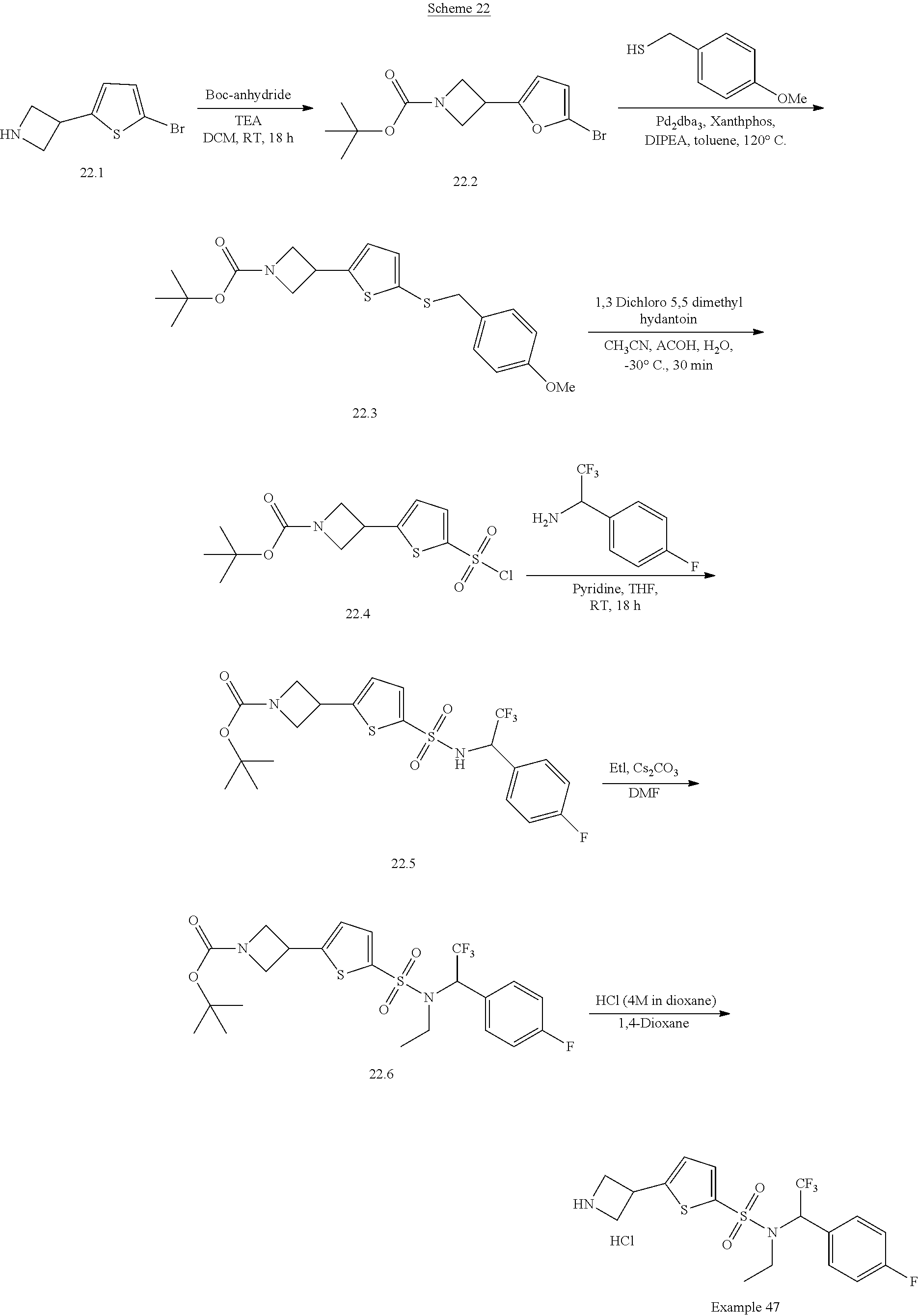

Synthesis of tert-butyl 3-(5-bromothiophen-2-yl)azetidine-1-carboxylate 22.2: To a stirred solution of 3-(5-bromothiophen-2-yl)azetidine 22.1 (0.4 g, 1.83 mmol) in DCM (4 mL), DIPEA (0.8 mL, 4.58 mmol) was added followed by di-tert-butyldicarbonate (0.51 g, 2.2 mmol) at 0° C. The overall reaction mixture was stirred at room temperature for 18 h. The reaction mixture was diluted with water (10 mL) and extracted with DCM (2×15 mL). The combined organic layer was dried over anhydrous sodium sulfate and concentrated under reduced pressure to afford crude compound which was then purified by combiflash column to afford tert-butyl 3-(5-bromothiophen-2-yl) azetidine-1-carboxylate as an off-white solid (0.45 g, 77%). ¹H NMR (400 MHz, DMSO-d₆): δ 7.09 (d, J=4.0 Hz, 1H), 6.91 (s, 1H), 4.35-4.15 (m, 2H), 4.06-3.94 (m, 1H), 3.86-3.72 (m, 2H), 1.39 (s, 9H).

Synthesis of tert-butyl 3-(5-((4-methoxybenzyl)thio)thiophen-2-yl)azetidine-1-carboxylate 22.3: Buchwald coupling: Method I: To a stirred solution of tert-butyl 3-(5-bromothiophen-2-yl)azetidine-1-carboxylate 22.2 (200 mg, 3.60 mmol) and (4-methoxyphenyl)methanethiol (0.5 mL, 3.60 mmol) in toluene (8 mL) DIPEA (2 mL, 10.81 mmol) was added and the solution was degassed with argon. Xantphos (36 mg, 0.06 mmol) was added to it followed by Pd₂(dba)₂ (58 mg, 0.06 mmol) under inert atmosphere. The mixture was heated to 80° C. and kept for 12 h. The reaction mixture was cooled, filtered through a celite bed and then concentrated under reduced pressure to afford crude compound which was purified by combiflash chromatography using 10% ethyl acetate-hexane to get tert-butyl 3-(5-((4-methoxybenzyl)thio)thiophen-2-yl)azetidine-1-carboxylate as pale yellow oil (0.21 mg, 85% yield). ¹H NMR (400 MHz, DMSO-d₆): δ 7.11 (d, J=12.0 Hz, 2H), 6.94-6.80 (m, 4H), 4.33-4.13 (m, 2H), 4.10-3.90 (m, 3H), 3.89-3.61 (m, 5H), 1.39 (s, 9H).

Synthesis of tert-butyl 3-(5-(chlorosulfonyl)thiophen-2-yl)azetidine-1-carboxylate 22.4: Sulfonyl chloride 22.4 was synthesized from Intermediate 22.3 following the procedure described in Method T. The crude sulfonyl chloride was used immediately in the forwarding step without further purification.

Synthesis of tert-butyl 3-(5-(N-(2,2,2-trifluoro-1-(4-fluorophenyl)ethyl)sulfamoyl)thiophen-2-yl)azetidine-1-carboxylate 21.5: Intermediate 22.5 was synthesized from sulfonyl chloride 22.4 following the protocol described in Method O, using 2,2,2-trifluoro-1-(4-fluorophenyl)ethan-1-amine hydrochloride, at 60° C. The compound was purified by column chromatography over silica gel using 35% ethyl acetate in hexane and pure compound tert-butyl 3-(5-(N-(2,2,2-trifluoro-1-(4-fluorophenyl)ethyl)sulfamoyl)thiophen-2-yl)azetidine-1-carboxylate was isolated as a light yellow gum (150 mg, 45% yield). ¹H NMR (400 MHz, Chloroform-d): δ 7.31 (d, J=12.8 Hz, 1H), 7.28-7.16 (m, 2H), 7.00 (t, J=8.4 Hz, 2H), 6.78 (d, J=3.7 Hz, 1H), 5.37 (d, J=9.0 Hz, 1H), 5.01-4.92 (m, 1H), 4.43-4.22 (m, 2H), 3.91-3.75 (m, 3H), 1.46 (s, 9H).

Synthesis of tert-butyl 3-(5-(N-ethyl-N-(2,2,2-trifluoro-1-(4-fluorophenyl)ethyl)sulfamoyl)thiophen-2-yl)azetidine-1-carboxylate 22.6: The ethylation of intermediate 22.6 was preformed following the protocol as described in Method D using Cs₂CO₃ at 60° C. The crude tert-butyl 3-(5-(N-ethyl-N-(2,2,2-trifluoro-1-(4-fluorophenyl)ethyl)sulfamoyl)thiophen-2-yl)azetidine-1-carboxylate (50 mg) was used in the forward step reaction.

Synthesis of 5-(azetidin-3-yl)-N-ethyl-N-(2,2,2-trifluoro-1-(4-fluorophenyl)ethyl)thiophene-2-sulfonamide hydrochloride salt (Example 47): To a stirred solution of compound tert-butyl 3-(5-(N-ethyl-N-(2,2,2-trifluoro-1-(4-fluorophenyl)ethyl)sulfamoyl)thiophen-2-yl)azetidine-1-carboxylate in 1,4-dioxane (2 mL) HCl (4M in dioxane) (0.5 mL) was added at 0° C. and then allowed to stir at ambient temperature for 18 h. The volatiles were evaporated under reduced pressure. The crude compound was purified by Reverse Phase Prep-HPLC purification to afford pure 5-(azetidin-3-yl)-N-ethyl-N-(2,2,2-trifluoro-1-(4-fluorophenyl)ethyl)thiophene-2-sulfonamide hydrochloride salt (25 mg, 40% yield) as a white solid. LCMS: m/z found 423.1 [M+H]⁺, rt=2.56 min (Method 9) [Waters Xbridge C18 column (3.5 μm, 50×3 mm))]. ¹H NMR (400 MHz, Methanol-d₄): δ 8.63-8.43 (m, 1H), 7.59 (dd, J=3.9, 9.4 Hz, 1H), 7.41 (q, J=8.0 Hz, 2H), 7.22-7.05 (m, 3H), 5.87-5.74 (m, 1H), 4.72-4.62 (m, 1H), 4.51-4.35 (m, 1H), 4.36-4.18 (m, 2H), 4.12-3.99 (m, 1H), 3.45-3.31 (m, 1H), 3.31-3.19 (m, 1H), 1.06 (q, J=6.8 Hz, 3H).

### Example 48 to 50

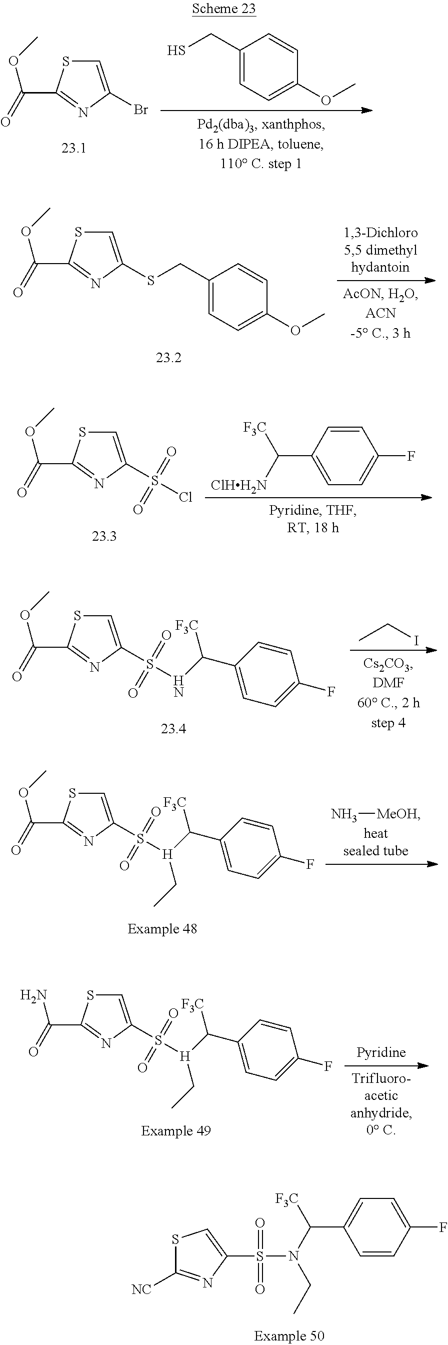

Synthesis of methyl 4-((4-methoxybenzyl)thio)thiazole-2-carboxylate 23.2: Intermediate 23.2 was synthesized from 4-bromothiazole-2-carboxylate 23.1 following a method analogous to that described in Method I using (4-methoxyphenyl)methanethiol. The crude was purified by column chromatography over silica gel using 20% ethyl acetate in hexane and methyl 4-((4-methoxybenzyl)-thio)thiazole-2-carboxylate (1.1 g, 82% yield) was isolated as brown sticky gum. ¹H NMR (400 MHz, DMSO-d₆): δ 7.83 (s, 1H), 7.26 (d, J=8.4 Hz, 2H), 6.86 (d, J=8.4 Hz, 2H), 4.28 (s, 2H), 3.91 (s, 3H), 3.71 (s, 3H).

Synthesis of methyl 4-(chlorosulfonyl)thiazole-2-carboxylate 23.3: Sulfonyl chloride 23.3 was synthesized from Intermediate 32 following the procedure described in Method T. The crude sulfonyl chloride was used immediately in the forwarding step without further purification.

Synthesis of methyl 4-(N-(2,2,2-trifluoro-1-(4-fluorophenyl)ethyl)sulfamoyl)thiazole-2-carboxylate 23.4: Intermediate 23.4 was synthesized from Sulfonyl chloride 23.3 following the protocol as described in Method O using 2,2,2-trifluoro-1-(4-fluorophenyl)ethan-1-amine hydrochloride at 60° C. The compound was purified by column chromatography over silica gel using 35% ethyl acetate in hexane and methyl 4-(N-(2,2,2-trifluoro-1-(4-fluorophenyl)ethyl)sulfamoyl)thiazole-2-carboxylate (220 mg, 33% yield) was isolated as brown gum. ¹H NMR (400 MHz, DMSO-d₆): δ 9.86-9.74 (m, 1H), 8.54 (s, 1H), 7.62-7.29 (m, 2H), 7.19-6.99 (m, 2H), 5.45-5.23 (m, 1H), 3.90 (s, 3H).

Synthesis of methyl 4-(N-ethyl-N-(2,2,2-trifluoro-1-(4-fluorophenyl)ethyl)sulfamoyl)thiazole-2-carboxylate (Example 48): The ethylation of intermediate 23.4 was preformed following the protocol as described in Method D at 60° C. The crude was purified by column chromatography over silica gel using 30% ethyl acetate in hexane and the desired compound 4-(N-ethyl-N-(2,2,2-trifluoro-1-(4-fluorophenyl)ethyl)sulfamoyl)thiazole-2-carboxylate (280 mg, 52% yield. 94.88% purity) was isolated as white solid. LCMS: m/z found 427.0 [M+H]⁺, rt=3 min (Method 9) [Waters Xbridge C18 column (3.5 μm, 50×3 mm))]. ¹H NMR (400 MHz, DMSO-d₆): δ 8.81 (s, 1H), 7.54 (dd, J=5.2, 8.6 Hz, 2H), 7.21 (t, J=8.7 Hz, 2H), 5.92 (q, J=8.7 Hz, 1H), 3.97 (s, 3H), 3.40-3.31 (m, 2H), 0.93 (t, J=7.0 Hz, 3H).

4-(N-ethyl-N-(2,2,2-trifluoro-1-(4-fluorophenyl)ethyl)sulfamoyl)thiazole-2-carboxamide (Example 49): In a 50 ml seal capped vial was added freshly prepared NH₃ in MeOH (0.2 ml) and to it was added compound 4-(N-ethyl-N-(2,2,2-trifluoro-1-(4-fluorophenyl)ethyl)sulfamoyl)thiazole-2-carboxylate (50 mg, 0.12 mmol) at ambient temperature and then heated to 60° C. for 2 h. Water (10 mL) was added to the reaction mass and then extracted with DCM (3×10 mL). The overall organic layer was dried over anhydrous MgSO4, filtered and concentrated under reduced pressure to afford crude compound which was purified by column chromatography using 30% EA in hexane to afford pure methyl 4-(N-ethyl-N-(2,2,2-trifluoro-1-(4-fluorophenyl)ethyl)sulfamoyl)thiazole-2-carboxamide (20 mg, 48% yield, 98.41% purity) as white solid. LCMS: m/z found 412.0 [M+H]⁺, rt=3 min (Method 9) [Waters Xbridge C18 column (3.5 μm, 50×3 mm))]; ¹H NMR (400 MHz, DMSO-d₆): δ 8.69 (s, 1H), 8.26 (s, 1H), 8.10 (s, 1H), 7.47 (dd, J=5.3, 8.6 Hz, 2H), 7.18 (t, J=8.8 Hz, 2H), 5.90 (q, J=8.7 Hz, 1H), 3.55-3.27 (m, 2H), 0.98 (t, J=7.0 Hz, 3H).

### Example 50

2-cyano-N-ethyl-N-(2,2,2-trifluoro-1-(4-fluorophenyl)ethyl)thiazole-4-sulfonamide: To a stirred solution of methyl 4-(N-ethyl-N-(2,2,2-trifluoro-1-(4-fluorophenyl)ethyl)sulfamoyl)thiazole-2-carboxamide (40 mg, 0.09 mmol) in pyridine (1 mL) was added trifluoroacetic anhydride (0.05 mL, 0.38 mmol) at −10° C. and stirred for 1 h. Water (10 mL) was added to the reaction and then extracted with EA (2×15 mL). The combined organic part was dried over anhydrous MgSO4 and concentrated under reduced pressure to afford crude compound which was purified by column chromatography over silica gel using 20% EA in hexane to afford 2-cyano-N-ethyl-N-(2,2,2-trifluoro-1-(4-fluorophenyl)ethyl)thiazole-4-sulfonamide (18 mg, 48% yield, 97.52% yield) as pale yellow sticky gum. LCMS: m/z found 392.0 [M−H], rt=3.03 min (Method 9) [Waters Xbridge C18 column (3.5 μm, 50×3 mm). ¹H NMR (400 MHz, DMSO-d₆) δ 8.87 (s, 1H), 7.45-7.37 (m, 2H), 7.23 (t, J=8.6 Hz, 2H), 5.92 (q, J=8.9 Hz, 1H), 3.50-3.32 (m, 2H), 1.00 (t, J=6.9 Hz, 3H).

### Examples 51, 52 and 53

### 5-(N-ethyl-N-(2,2,2-trifluoro-1-(4-fluorophenyl)ethyl)sulfamoyl)nicotinamide (Example 51), (S)-5-(N-ethyl-N-(2,2,2-trifluoro-1-(4-fluorophenyl)ethyl)sulfamoyl)nicotinamide (Example 52) and (R)-5-(N-ethyl-N-(2,2,2-trifluoro-1-(4-fluorophenyl)ethyl)sulfamoyl)nicotinamide (Example 53)

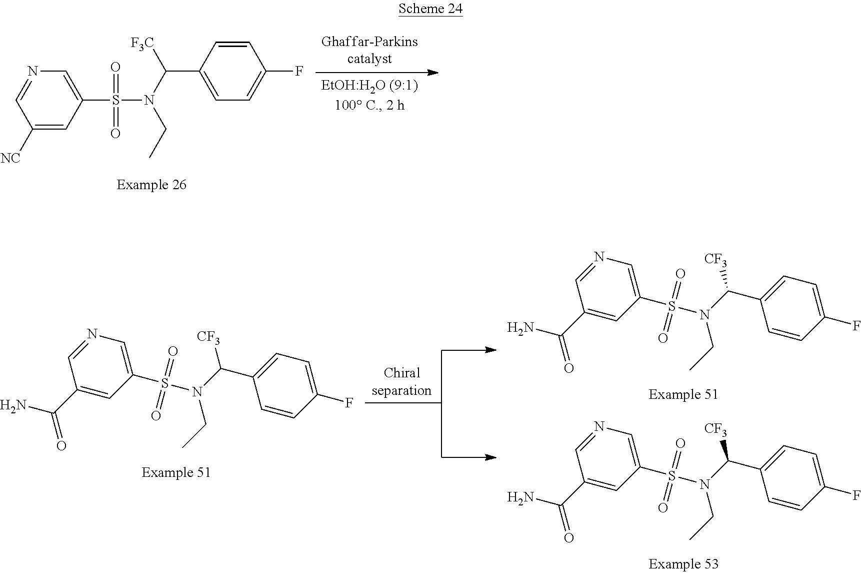 [SMILES: CCN(C(c1ccc(F)cc1)C(F)(F)F)S(=O)(=O)c1cncc(C#N)c1 ; CCN(C(c1ccc(F)cc1)C(F)(F)F)S(=O)(=O)c1cncc(C(N)=O)c1 ; CCN([C@@H](c1ccc(F)cc1)C(F)(F)F)S(=O)(=O)c1cncc(C(N)=O)c1 ; CCN([C@H](c1ccc(F)cc1)C(F)(F)F)S(=O)(=O)c1cncc(C(N)=O)c1]

Example 26 was hydrolyzed to compound Example 51 following the protocol described in Method N. The crude product was purified by column chromatography using 30% ethyl acetate in hexane. Example 51 was isolated as an off-white sticky solid (210 mg, 71% yield, 99.06% purity). LCMS: m/z found 406.1 [M+H]⁺, rt=2.56 min (Method 9) [Waters Xbridge C18 column (3.5 μm, 50×3 mm)]; ¹H NMR (400 MHz, DMSO-d₆): δ 9.23 (dd, J=2.1, 18.5 Hz, 2H), 8.65 (t, J=2.1 Hz, 1H), 8.39 (s, 1H), 7.86 (s, 1H), 7.46 (dd, J=5.3, 8.5 Hz, 2H), 7.23 (t, J=8.8 Hz, 2H), 6.09 (q, J=8.4 Hz, 1H), 3.47-3.24 (m, 2H), 0.97 (t, J=7.0 Hz, 3H).

Chiral separation of racemic Example 51 provided both the enantiomers as described below. Chiral separation method: chiral separation was performed on an Agilent 1200 series instrument. Column: CHIRALPAK IA (250×20 mm) 5u, operating at ambient temperature and at a flow rate of 18.0 mL/min. Mobile phase was mixture of 85% Hexane, 15% ETOH and 0.1% IP amine, held this isocratic mixture run up to 25 min with wavelength of 220 nm.

Example 52: off-white solid. 99.74% Purity. LCMS: m/z found 406.1 [M+H], rt=2.57 min (Method 9) [Waters Xbridge C18 column (3.5 μm, 50×3 mm). ¹H NMR (400 MHz, DMSO-d₆) δ 9.28-9.18 (m, 2H), 8.64 (s, 1H), 8.39 (s, 1H), 7.86 (s, 1H), 7.53-7.39 (m, 2H), 7.23 (t, J=8.7 Hz, 2H), 6.09 (q, J=8.4 Hz, 1H), 3.49-3.27 (m, 2H), 0.97 (t, J=6.9 Hz, 3H).

Example 53: beige solid. 99.50% Purity. LCMS: m/z found 406.1 [M+H], rt=2.57 min (Method 9) [Waters Xbridge C18 column (3.5 μm, 50×3 mm). ¹H NMR (400 MHz, DMSO-d₆) δ 9.28-9.18 (m, 2H), 8.64 (s, 1H), 8.39 (s, 1H), 7.86 (s, 1H), 7.53-7.39 (m, 2H), 7.23 (t, J=8.7 Hz, 2H), 6.09 (q, J=8.4 Hz, 1H), 3.49-3.27 (m, 2H), 0.97 (t, J=6.9 Hz, 3H).

### Example 54

### N-(1-(4-chlorophenyl)-2,2,2-trifluoroethyl)-5-cyano-N-ethylpyridine-3-sulfonamide

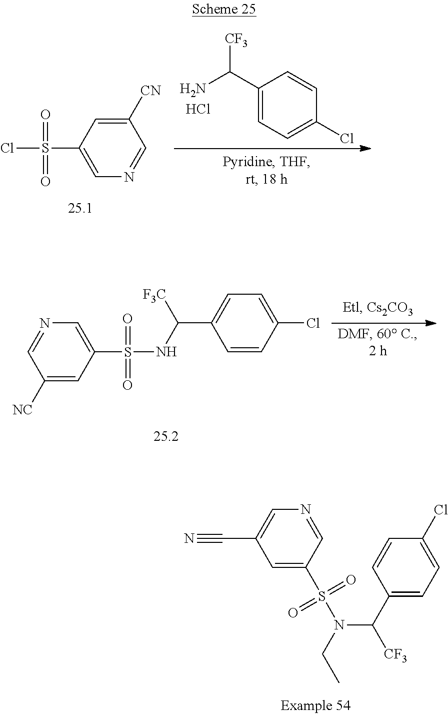 [SMILES: N#Cc1cncc(S(=O)(=O)Cl)c1 ; Cl ; NC(c1ccc(Cl)cc1)C(F)(F)F ; N#Cc1cncc(S(=O)(=O)NC(c2ccc(Cl)cc2)C(F)(F)F)c1 ; "DMF, 60° C.,2 h" ; CCN(C(c1ccc(Cl)cc1)C(F)(F)F)S(=O)(=O)c1cncc(C#N)c1]

Synthesis of N-(1-(4-chlorophenyl)-2,2,2-trifluoroethyl)-5-cyanopyridine-3-sulfonamide 25.2: Intermediate 25.2 was synthesized from 6-cyanopyridine-3-sulfonyl chloride 25.1 following the protocol as described in Method O using 1-(4-chlorophenyl)-2,2,2-trifluoroethan-1-amine hydrochloride. The compound was purified by column chromatography over silica gel using 30% ethyl acetate in hexane and N-(1-(4-chlorophenyl)-2,2,2-trifluoroethyl)-5-cyanopyridine-3-sulfonamide (550 mg, 59.4% yield) was isolated as light-yellow sticky oil. ¹H NMR (400 MHz, DMSO-d₆): δ 10.33-9.40 (m, 1H), 9.15 (d, J=1.6 Hz, 1H), 9.02 (d, J=2.0 Hz, 1H), 8.44 (t, J=1.6 Hz, 1H), 7.60-7.28 (m, 4H), 5.52 (q, J=7.6 Hz, 1H).

Synthesis of N-(1-(4-chlorophenyl)-2,2,2-trifluoroethyl)-5-cyano-N-ethylpyridine-3-sulfonamide (Example 54): The ethylation of intermediate 25.2 was performed following the protocol as described in Method D at 60° C. The crude residue was purified by column chromatography over silica gel using 15% ethyl acetate in hexane to afford N-(1-(4-chlorophenyl)-2,2,2-trifluoroethyl)-5-cyano-N-ethylpyridine-3-sulfonamide (131 mg, 24% yield. 95.96% purity) as a pale-yellow sticky liquid. LCMS: m/z found 404.1 [M+H]⁺, rt=3.68 min (Method 4) [Waters Xbridge C18 column (5 μm, 50×4.6 mm). ¹H NMR (400 MHz, DMSO-d₆): δ 9.32 (dd, J=2.1, 9.9 Hz, 2H), 8.91 (t, J=2.1 Hz, 1H), 7.49 (d, J=8.4 Hz, 2H), 7.40 (d, J=8.8 Hz, 2H), 6.10 (q, J=8.4 Hz, 1H), 3.54-3.26 (m, 2H), 1.00 (t, J=6.8 Hz, 3H).

### Example 55

### 5-cyano-N-ethyl-6-methyl-(2,2,2-trifluoro-1-(4-fluorophenyl)ethyl)pyridine-3-sulfonamide

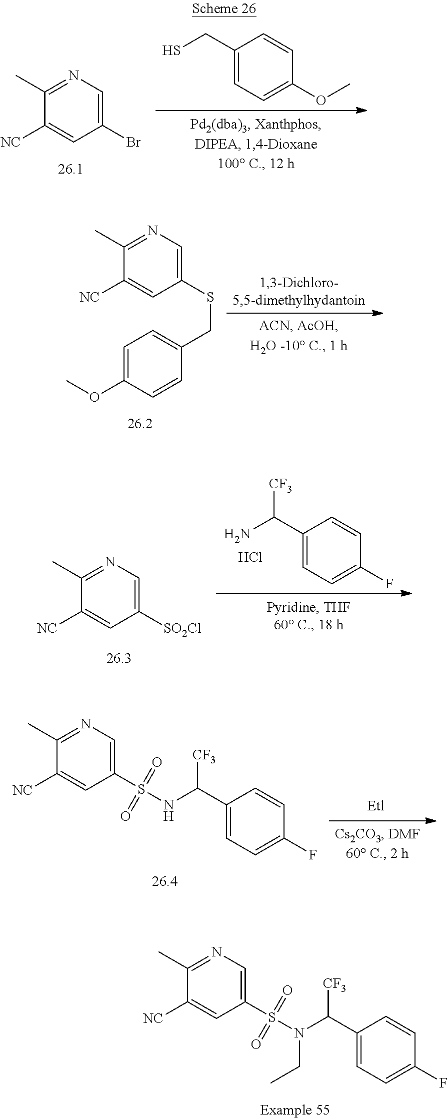

Synthesis of 5-((4-methoxybenzyl)thio)nicotinonitrile 26.2 Intermediate 26.2 was synthesized from 4-bromothiazole-2-carboxylate 26.1 following a method analogous to that described in Method I using (4-methoxyphenyl)methanethiol. The crude was purified by column chromatography over silica gel using 20% ethyl acetate in hexane to get 5-((4-methoxybenzyl)thio)-2-methylnicotinonitrile (258 mg, 75% yield) as yellow gum. ¹H NMR (400 MHz, DMSO-d₆): δ 8.58 (s, 1H), 8.23 (s, 1H), 7.26 (d, J=8.2 Hz, 2H), 6.86 (d, J=8.1 Hz, 2H), 4.28 (s, 2H), 3.71 (s, 3H), 2.60 (s, 3H).

Synthesis of 5-cyano-6-methylpyridine-3-sulfonyl chloride 26.3: Sulfonyl chloride 26.3 was synthesized from intermediate 26.2 following the procedure described in Method T. The crude sulfonyl chloride was used immediately in the forwarding step without any further purification.

Synthesis of 5-cyano-6-methyl-N-(2,2,2-trifluoro-1-(4-fluorophenyl)ethyl)pyridine-3-sulfonamide 26.4: Intermediate 26.4 was synthesized from sulfonyl chloride 26.3 following the protocol as described in Method O using 2,2,2-trifluoro-1-(4-fluorophenyl)ethan-1-amine hydrochloride at 60° C. The compound was purified by column chromatography over silica gel using 35% ethyl acetate in hexane to afford 5-cyano-6-methyl-N-(2,2,2-trifluoro-1-(4-fluorophenyl)ethyl)pyridine-3-sulfonamide (80 mg, 29% yield) as yellow gum. 1H NMR (400 MHz, DMSO-d₆): δ 9.70 (d, J=6.9 Hz, 1H), 8.84 (d, J=2.1 Hz, 1H), 8.30 (d, J=1.8 Hz, 1H), 7.43 (dd, J=5.3, 8.6 Hz, 2H), 7.12 (t, J=8.8 Hz, 2H), 5.80-5.20 (m, 1H), 2.66 (s, 3H).

Synthesis of 5-cyano-N-ethyl-6-methyl-N-(2,2,2-trifluoro-1-(4-fluorophenyl)ethyl)pyridine-3-sulfonamide (Example 55): The ethylation of intermediate 26.4 was preformed following the protocol as described in Method D at 60° C. The crude was purified by column chromatography over silica gel using 30% ethyl acetate in hexane and the desired compound Example 55 (160 mg, 29% yield. 97.8% purity) was isolated as pale-yellow sticky liquid. LCMS: m/z found 400.1 [M+H]⁺, rt=2.97 min (Method 9) [Waters Xbridge C18 column (3.5 μm, 50×3 mm). ¹H NMR (400 MHz, DMSO-d₆): δ 9.17 (s, 1H), 8.79 (s, 1H), 7.45 (t, J=5.3 Hz, 2H), 7.26 (t, J=8.7 Hz, 2H), 6.06 (q, J=9.0 Hz, 1H), 3.64-3.34 (m, 2H), 2.77 (s, 3H), 0.98 (t, J=6.9 Hz, 3H).

### Example 56

### N-ethyl-N-(2,2,2-trifluoro-1-(4-fluorophenyl)ethyl)tetrahydro-2H-pyran-3-sulfonamide

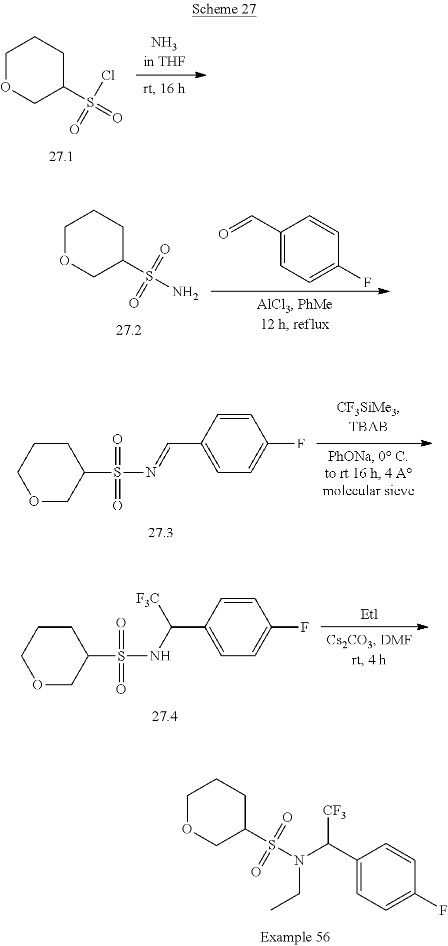 [SMILES: O=S(=O)(Cl)C1CCCOC1 ; NS(=O)(=O)C1CCCOC1 ; O=Cc1ccc(F)cc1 ; O=S(=O)(/N=C/c1ccc(F)cc1)C1CCCOC1 ; O=S(=O)(NC(c1ccc(F)cc1)C(F)(F)F)C1CCCOC1 ; CCN(C(c1ccc(F)cc1)C(F)(F)F)S(=O)(=O)C1CCCOC1]

Synthesis of tetrahydro-2H-pyran-3-sulfonamide 27.2: Sulfonamidation by Method O: To a stirred solution of tetrahydro-2H-pyran-3-sulfonyl chloride 27.1 (400 mg, 2.16 mmol) in THF (1 mL) was added freshly prepared NH₃ in THF (20 mL) at 0° C. and the mixture was stirred at room temperature for 16 h. The reaction was then filtered through celite, and the residue was collected and concentrated to a residue. The residue was purified by column chromatography over silica gel using 5:1 ethyl acetate-hexane. Intermediate 27.2 was obtained as white solid (201 mg, 56% yield). ¹H NMR (400 MHz, DMSO-d₆): δ 6.75 (s, 2H), 3.93 (dd, J=4.6, 11.5 Hz, 2H), 3.41-3.22 (m, 2H), 3.12-2.98 (m, 1H), 1.92-1.83 (m, 2H), 1.68-1.52 (m, 2H).

Synthesis of N-(4-fluorobenzylidene)tetrahydro-2H-pyran-3-sulfonamide 27.3: Imine formation by Method P: To a stirred solution of tetrahydro-2H-pyran-3-sulfonamide 27.2 (200 mg, 1.21 mmol) and 4-fluorobenzaldehyde (180 mg, 1.45 mmol) in toluene (2 mL) was added AlCl₃ (48 mg, 0.36 mmol) and refluxed for 12 h. The reaction was then filtered and the solution was concentrated under reduced pressure. The crude 27.3 was directly used in the forwarding step without any further purification.

Synthesis of N-(2,2,2-trifluoro-1-(4-fluorophenyl)ethyl)tetrahydro-2H-pyran-3-sulfonamide 27.4: Insertion of —CF₃ group: Method Q: To a stirred solution of intermediate 27.3 (100 mg, 0.37 mmol) in toluene (2 mL) under N₂ atmosphere was added TBAB (7 mg, 0.02 mmol) followed by 4A molecular sieves. The reaction mixture was cooled at 0° C. and CF₃SiMe₃ (78 mg, 0.55 mmol) was added to it followed by sodium phenoxide (38 mg, 0.55 mmol). Stirring was continued at room temperature for 16 h. The reaction was quenched with saturated aqueous Na₂CO₃ solution (5 mL) and extracted with ethyl acetate (2×15 mL). Combined organic layers were washed with water (10 mL) and brine (10 mL), dried over anhydrous Na₂SO₄ and the solvent was evaporated under reduced pressure. The crude was purified by column chromatography over silica gel using 1:5 ethyl acetate-hexane. Intermediate 27.4 was obtained as white solid (40 mg, 32% yield). LCMS: m/z found 342.2 [M+H]⁺, rt=3.25 min (Method 4) [Xbridge C18 column (5 μm, 50×4.6 mm)].

Synthesis of N-ethyl-N-(2,2,2-trifluoro-1-(4-fluorophenyl)ethyl)tetrahydro-2H-pyran-3-sulfonamide (Example 56): The ethylation of intermediate 27.4 was preformed following the protocol as described in Method D using Cs₂CO₃. The crude was purified by column chromatography over silica gel using 25% ethyl acetate in hexane and Example 56 was isolated as colorless oil (20 mg, 31% yield, 93.64% purity). LCMS: m/z found (370.2 [M+H]⁺, rt=9.42 min (Method 6) [Xbridge C18 column (5 μm, 50×4.6 mm)]; ¹H NMR (400 MHz, DMSO-d₆): δ 7.66 (q, J=6.3 Hz, 2H), 7.39-7.29 (m, 2H), 5.92-5.77 (m, 1H), 3.98 (t, J=10.9 Hz, 1H), 3.78 (d, J=11.9 Hz, 1H), 3.64-3.39 (m, 2H), 3.31-3.16 (m, 1H), 2.12-1.87 (m, 1H), 1.84-1.64 (m, 4H), 1.49 (dd, J=12.8, 17.1 Hz, 1H), 0.95 (t, J=6.9 Hz, 3H).

### Example 57 and Example 58

### N-ethyl-N-(2,2,2-trifluoro-1-(4-fluorophenyl)ethyl)pyrimidine-5-sulfonamide (Example 57) and N-methyl-N-(2,2,2-trifluoro-1-(4-fluorophenyl)ethyl)pyrimidine-5-sulfonamide (Example 58

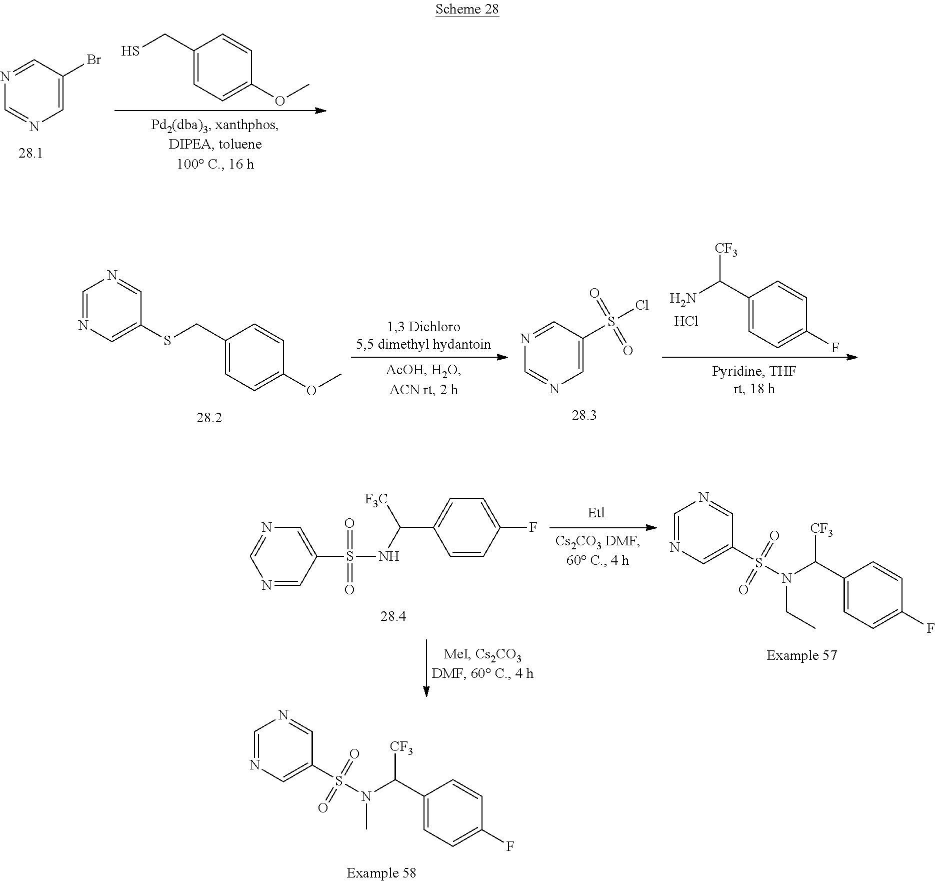

Synthesis of 5-((4-methoxybenzyl)thio)pyrimidine 28.2: Intermediate 28.2 was synthesized from 5-bromopyrimidine 28.1 following a method analogous to that described in Method I using (4-methoxyphenyl)methanethiol. The crude 28.2 was directly used in the forwarding step without further purification.

Synthesis of pyrimidine-5-sulfonyl chloride 28.3: Sulfonyl chloride 28.3 was synthesized from intermediate 28.2 following the procedure described in Method T. The crude sulfonyl chloride was used immediately in the forwarding step without further purification.

Synthesis of N-(2,2,2-trifluoro-1-(4-fluorophenyl)ethyl)pyrimidine-5-sulfonamide 28.4: Intermediate 28.4 was synthesized from sulfonyl chloride 28.3 following the protocol as described in Method O using 2,2,2-trifluoro-1-(4-fluorophenyl)ethan-1-amine hydrochloride. The compound was purified by column chromatography over silica gel using 35% ethyl acetate in hexane and isolated as white sticky solid (204 mg, 31%). LCMS: m/z found (336.0 [M+H+]), rt=2.57 min (Method 9) [Xbridge C18 column (5 μm, 50×4.6 mm)].

Synthesis of N-ethyl-N-(2,2,2-trifluoro-1-(4-fluorophenyl)ethyl)pyrimidine-5-sulfonamide (Example 57): The ethylation of intermediate 28.4 was performed following the protocol as described in Method D at 60° C. The crude was purified by column chromatography over silica gel using 30% ethyl acetate in hexane and the desired compound was isolated as colourless oil (10 mg, 26% yield, 97.71% purity). LCMS: m/z found 364.0 [M+H]⁺, rt=2.85 min (Method 9) [Xbridge C18 column (3.5 μm, 50×3 mm)]; ¹H NMR (400 MHz, Chloroform-d): δ 9.39 (s, 1H), 9.16 (s, 2H), 7.49 (dd, J=5.0, 8.5 Hz, 2H), 7.12 (t, J=8.6 Hz, 2H), 5.81 (q, J=8.2 Hz, 1H), 3.45-3.33 (m, 1H), 3.29-3.14 (m, 1H), 0.95 (t, J=7.1 Hz, 3H).

Synthesis of N-methyl-N-(2,2,2-trifluoro-1-(4-fluorophenyl)ethyl)pyrimidine-5-sulfonamide (Example 58): The methylation of intermediate 28.4 was preformed following the protocol as described in Method D at 60° C. The crude was purified by column chromatography over silica gel using 30% ethyl acetate in hexane and the desired compound was isolated as colourless oil (11 mg, 30% yield). ¹H NMR (400 MHz, Chloroform-d) δ 9.40 (s, 1H), 9.12 (s, 2H), 7.44 (dd, J=5.1, 8.6 Hz, 2H), 7.13 (t, J=8.6 Hz, 2H), 5.87 (q, J=8.2 Hz, 1H), 2.77 (s, 3H).

### Example 59 and Example 60

### (S)—N-ethyl-N-(2,2,2-trifluoro-1-(4-fluorophenyl)ethyl)pyrimidine-5-sulfonamide (Example 59) and (R)—N-ethyl-N-(2,2,2-trifluoro-1-(4-fluorophenyl)ethyl)pyrimidine-5-sulfonamide (Example 60)

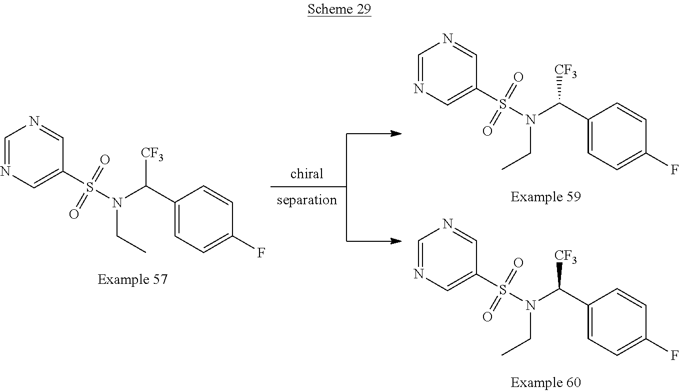 [SMILES: CCN(C(c1ccc(F)cc1)C(F)(F)F)S(=O)(=O)c1cncnc1 ; CCN([C@@H](c1ccc(F)cc1)C(F)(F)F)S(=O)(=O)c1cncnc1 ; CCN([C@H](c1ccc(F)cc1)C(F)(F)F)S(=O)(=O)c1cncnc1]

Chiral separation of racemic Example 57 provided both enantiomers as described below. Chiral separation was done on Agilent 1200 series instrument. Column name: CHIRALPAK IA (250×20 mm) 5 μm, operating at ambient temperature and at a flow rate of 18.0 mL/min. Mobile phase was mixture of 85% Hexane and 15% ETOH, held this isocratic mixture run up to 25 min with wavelength of 225 nm.

Example 59: Colourless oil (15 mg, 97.97% purity). LCMS: m/z found (364.2 [M+H+]), rt=3.28 min (Method 2) Acquity BEH C18 column (1.7 μm, 50×2.1 mm); ¹H NMR (400 MHz, Chloroform-d): δ 9.38 (s, 1H), 9.14 (s, 2H), 7.55-7.44 (m, 2H), 7.12 (t, J=8.6 Hz, 2H), 5.81 (q, J=8.3 Hz, 1H), 3.46-3.33 (m, 1H), 3.29-3.16 (m, 1H), 0.94 (t, J=7.1 Hz, 3H).

Example 60: Colourless oil (12 mg, 95.11% purity). LCMS: m/z found (364.2 [M+H+]), rt=3.28 min (Method 2) Acquity BEH C18 column (1.7 μm, 50×2.1 mm); ¹H NMR (400 MHz, Chloroform-d): δ 9.38 (s, 1H), 9.14 (s, 2H), 7.49 (dd, J=5.1, 8.2 Hz, 2H), 7.12 (t, J=8.4 Hz, 2H), 5.81 (q, J=8.4 Hz, 1H), 3.45-3.33 (m, 1H), 3.29-3.14 (m, 1H), 0.94 (t, J=7.1 Hz, 3H).

### Examples 61, 62 and 63

### N-ethyl-N-(2,2,2-trifluoro-1-(4-fluorophenyl)ethyl)tetrahydro-2H-pyran-4-sulfonamide (Example 61), (S)—N-ethyl-N-(2,2,2-trifluoro-1-(4-fluorophenyl)ethyl)tetrahydro-2H-pyran-4-sulfonamide (Example 62) and (R)—N-ethyl-N-(2,2,2-trifluoro-1-(4-fluorophenyl)ethyl)tetrahydro-2H-pyran-4-sulfonamide (Example 63)

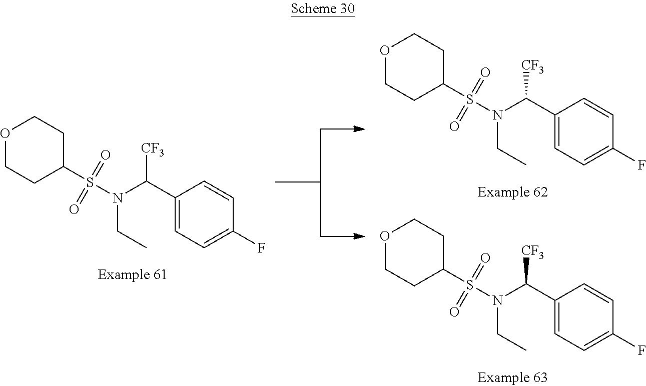 [SMILES: CCN(C(c1ccc(F)cc1)C(F)(F)F)S(=O)(=O)C1CCOCC1 ; CCN([C@@H](c1ccc(F)cc1)C(F)(F)F)S(=O)(=O)C1CCOCC1 ; CCN([C@H](c1ccc(F)cc1)C(F)(F)F)S(=O)(=O)C1CCOCC1]

Example 61 was prepared was prepared using an analogous method to Example 130, using intermediate 65.3 as the starting material and Method D for the final ethylation step.

Chiral separation of racemic Example 61 provided both the enantiomers as mentioned below. Chiral separation of CR658-18290-9-A-SFC was done on Thar SFC-80 series instrument by using C-Amylose A column (30 mm×250 mm), 5μ, operating at 35° C. temperature, maintaining flow rate of 40 gm/min using 80% C02 in super critical state & 20% of 0.3% Isopropylamine in Methanol as mobile phase, Run this isocratic mixture up to 8 min and also maintained the isobaric condition of 100 bar at 213 nm wavelength.

Example 62: Colorless oil (21 mg, 99.39% purity). ¹H NMR (400 MHz, DMSO-d₆) δ 7.65 (t, J=7.4 Hz, 2H), 7.33 (t, J=8.6 Hz, 2H), 5.80 (q, J=8.6 Hz, 1H), 4.06-3.86 (m, 2H), 3.66-3.53 (m, 1H), 3.31-3.15 (m, 2H), 1.83-1.57 (m, 6H), 0.95 (t, J=6.9 Hz, 3H).

Example 63: Colorless oil (22 mg, 99.87% purity). ¹H NMR (400 MHz, DMSO-d₆) δ 7.65 (t, J=7.3 Hz, 2H), 7.33 (t, J=8.7 Hz, 2H), 5.80 (q, J=8.7 Hz, 1H), 4.08-3.87 (m, 2H), 3.66-3.53 (m, 1H), 3.31-3.15 (m, 2H), 1.83-1.57 (m, 6H), 0.95 (t, J=6.9 Hz, 3H).

### Example 64 to Example 66

### 5-Cyano-N-(2-fluoroethyl)-N-(2,2,2-trifluoro-1-(4-fluorophenyl)ethyl)pyridine-3-sulfonamide (Example 64) (S)-5-Cyano-N-(2-fluoroethyl)-N-(2,2,2-trifluoro-1-(4-fluorophenyl)ethyl)pyridine-3-sulfonamide (Example 65) and (R)-5-cyano-N-(2-fluoroethyl)-N-(2,2,2-trifluoro-1-(4-fluorophenyl)ethyl)pyridine-3-sulfonamide (Example 66)

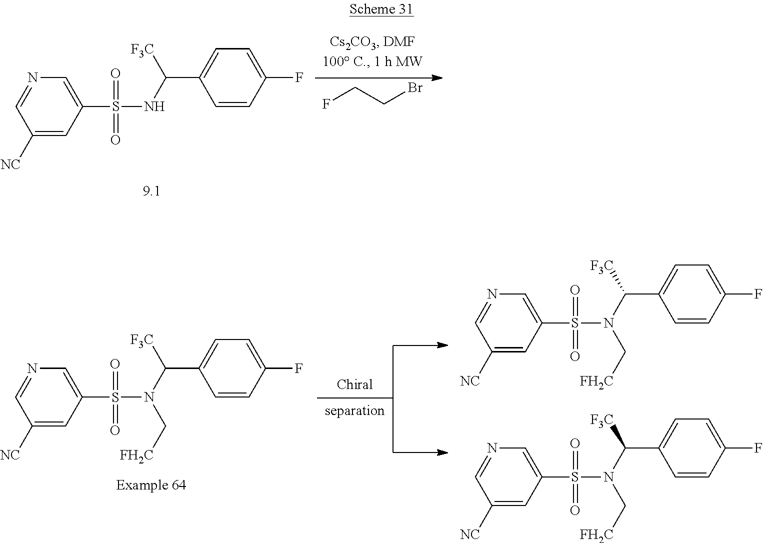

5-Cyano-N-(2-fluoroethyl)-N-(2,2,2-trifluoro-1-(4-fluorophenyl)ethyl)pyridine-3-sulfonamide (Example 64): Method R; Alkylation under MW: To the stirred solution of 9.1 (150 mg, 0.328 mmol) in DMF (2 mL), Cs₂CO₃ (319 mg, 0.983 mmol) was added at RT under argon atmosphere and the reaction mixture was stirred for 10 min at RT. 1-bromo-2-fluoroethane (0.2 mL, 1.638 mmol) was added to the reaction mixture and the reaction mixture was irradiated under MW at 100° C. temperature for 1 h. The crude reaction mass was diluted with water (10 mL) and extracted with ethyl acetate (3×10 mL). The combined organic part was washed with water (2×10 mL) and brine (10 mL), dried (anhydrous Na₂SO₄) and the solvent was evaporated under reduced pressure. The crude (150 mg) was purified by column chromatography over silica gel using 30% ethyl acetate in hexane and Example 64 was isolated as a light-yellow sticky gum (20 mg, 15% yield, 97.66% purity). LCMS: m/z found 406.1 [M+H]⁺, rt=3.56 min (Method 4) [Waters Xbridge C18 column (5 μm, 50×4.6 mm)]; ¹H NMR (400 MHz, DMSO-d₆) δ 9.31 (d, J=2.0 Hz, 2H), 8.87 (t, J=1.8 Hz, 1H), 7.52-7.39 (m, 2H), 7.27 (t, J=8.8 Hz, 2H), 6.16 (q, J=8.4 Hz, 1H), 4.53-4.41 (m, 1H), 4.40-4.30 (m, 1H), 3.84-3.54 (m, 2H).

Chiral separation of racemic Example 64 provided both the enantiomers as mentioned below. Chiral separation method: Chiral separation was done on Agilent 1200 series instrument. Column name—CHIRALPAK IA (250×20 mm) 5u. Operating at ambient temperature and flow rate is 18.0 mL/min. Mobile phase was a mixture of 80% Hexane, 20% ETOH and 0.1% isopropylamine, held this isocratic mixture run up to 26 min with wavelength of 220 nm.

Example 65: White Solid, 98.51% purity. ¹H NMR (400 MHz, DMSO-d₆): δ 9.30 (d, J=1.6 Hz, 2H), 8.87 (t, J=2.1 Hz, 1H), 7.46 (dd, J=5.3, 8.6 Hz, 2H), 7.27 (t, J=8.8 Hz, 2H), 6.16 (q, J=8.4 Hz, 1H), 4.59-4.24 (m, 2H), 3.86-3.54 (m, 2H).

Example 66: White Solid, 97.78% purity. ¹H NMR (400 MHz, DMSO-d₆): δ 9.30 (d, J=1.6 Hz, 2H), 8.88 (t, J=2.1 Hz, 1H), 7.46 (dd, J=5.3, 8.6 Hz, 2H), 7.27 (t, J=8.8 Hz, 2H), 6.15 (q, J=8.3 Hz, 1H), 4.63-4.23 (m, 2H), 3.83-3.56 (m).

### Example 67 to Example 69

### 5-Cyano-N-methyl-N-(2,2,2-trifluoro-1-(4-(trifluoromethyl)phenyl)ethyl)pyridine-3-sulfonamide (Example 67), (R)-5-Cyano-N-methyl-N-(2,2,2-trifluoro-1-(4-(trifluoromethyl)phenyl)ethyl)pyridine-3-sulfonamide (Example 68) and (S)-5-Cyano-N-methyl-N-(2,2,2-trifluoro-1-(4-(trifluoromethyl)phenyl)ethyl)pyridine-3-sulfonamide (Example 69)

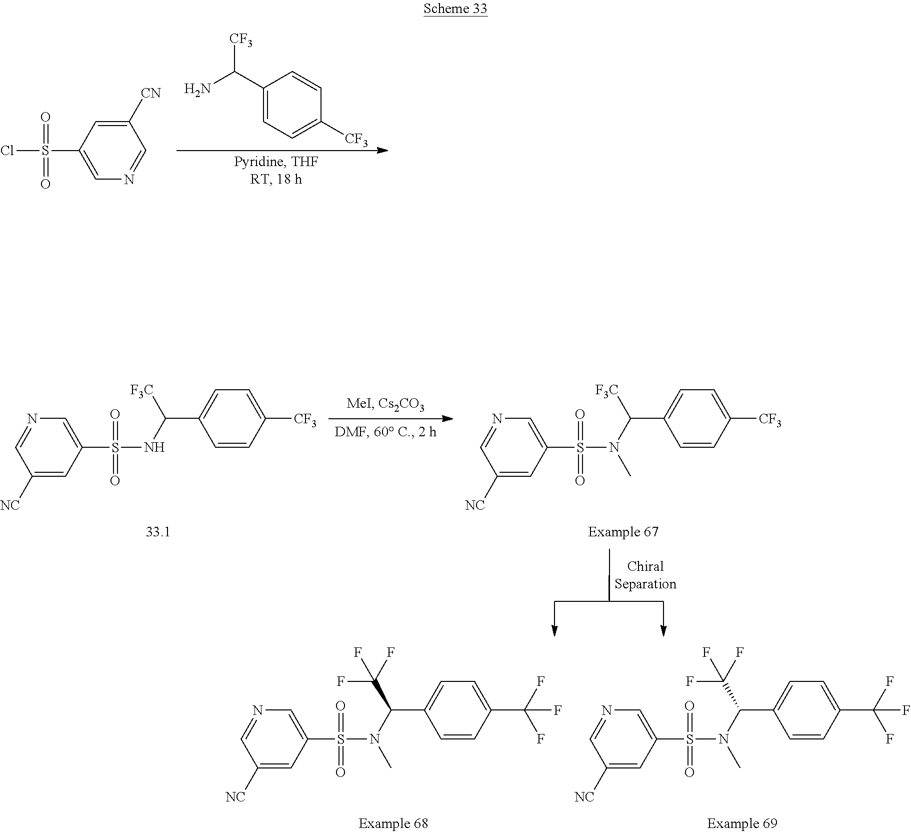

Synthesis of 5-cyano-N-(2,2,2-trifluoro-1-(4-(trifluoromethyl)phenyl)ethyl)pyridine-3-sulfonamide 33.1: Intermediate 33.1 was synthesized from 5-cyanopyridine-3-sulfonyl chloride following the protocol as described in Method O using 2,2,2-trifluoro-1-(4-(trifluoromethyl)phenyl)ethan-1-amine hydrochloride. The compound was purified by column chromatography over silica gel using 35% ethyl acetate in hexane and isolated as yellow sticky gum (300 mg, 50% yield). ¹H NMR (400 MHz, DMSO-d₆): δ 9.12 (d, J=1.4 Hz, 1H), 9.03 (d, J=2.0 Hz, 1H), 8.45 (t, J=2.1 Hz, 1H), 7.81-7.70 (m, 1H), 7.72-7.60 (m, 4H), 5.67 (q, J=8.1, 8.6 Hz, 1H).

Synthesis of 5-cyano-N-methyl-N-(2,2,2-trifluoro-1-(4-(trifluoromethyl)phenyl)ethyl)pyridine-3-sulfonamide (Example 67): The methylation of intermediate 33.1 was performed following the protocol as described in Method D at 60° C. The crude residue was purified by column chromatography over silica gel using 5:1 ethyl acetate-hexane and the compound Example 67 was obtained as a colourless, sticky gum (80 mg, 52% yield, 99.84% purity). LCMS: m/z found 424.2 [M+H+], rt=3.20 min (Method 2) [Acquity BEH C18 column (1.7 μm, 50×2.1 mm)]; ¹H NMR (400 MHz, DMSO-d₆): δ 9.33 (t, J=2.2 Hz, 2H), 8.91 (t, J=2.0 Hz, 1H), 7.81 (d, J=8.0 Hz, 2H), 7.60 (d, J=7.6 Hz, 2H), 6.25 (q, J=8.4 Hz, 1H), 2.90 (s, 3H).

### (R)-5-Cyano-N-methyl-N-(2,2,2-trifluoro-1-(4-(trifluoromethyl)phenyl)ethyl)pyridine-3-sulfonamide (Example 68) and (S)-5-Cyano-N-methyl-N-(2,2,2-trifluoro-1-(4-(trifluoromethyl)phenyl)ethyl)pyridine-3-sulfonamide (Example 69)

Chiral separation of racemic Example 67 provided both the enantiomers as mentioned below. Chiral separation was done on Thar SFC-80 series instrument by using C-Amylose A column (30 mm×250 mm), 5μ, operating at 35° C. temperature, maintaining flow rate of 60 g/min, using 80% CO₂ in super critical state & 20% of (MeOH/Hex/i-propylamine (70/20/10)) as mobile phase, Run this isocratic mixture up to 11 min and also maintained the isobaric condition of 100 bar at 220 nm wavelength.

Example 68 (EN-1): Colourless sticky gum (98.35% purity). LCMS: m/z found 424.2 [M+H]⁺, rt=3.16 min (Method 2) [Acquity BEH C18 column (1.7 μm, 50×2.1 mm)]; ¹H NMR (400 MHz, DMSO-d₆): δ 9.33 (t, J=1.7 Hz, 2H), 8.90 (t, J=1.7 Hz, 1H), 7.81 (d, J=8.3 Hz, 2H), 7.60 (d, J=8.1 Hz, 2H), 6.25 (q, J=8.3 Hz, 1H), 2.89 (s, 3H).

Example 69 (EN-2): Colourless sticky gum (97.85% purity). LCMS: m/z found 424.1 [M+H]⁺, rt=3.16 min (Method 2) [Acquity BEH C18 column (1.7 μm, 50×2.1 mm)]; ¹H NMR (400 MHz, DMSO-d₆): δ 9.33 (t, J=2.0 Hz, 2H), 8.90 (t, J=2.0 Hz, 1H), 7.81 (d, J=8.2 Hz, 2H), 7.60 (d, J=8.1 Hz, 2H), 6.25 (q, J=8.3 Hz, 1H), 2.89 (s, 3H).

### Example 70

### N-ethyl-6-methoxy-N-(2,2,2-trifluoro-1-(4-fluorophenyl)ethyl)pyridazine-4-sulfonamide

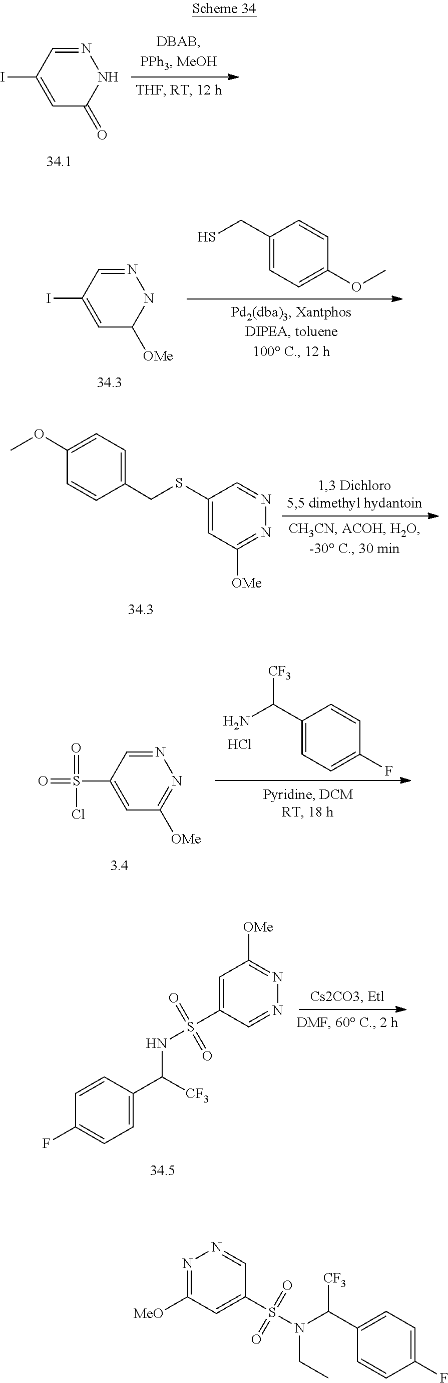

Synthesis of 5-iodo-3-methoxypyridazine 34.2: To a solution of 5-iodopyridazin-3(2H)-one (34.1; 3.0 g, 13.57 mmol) in THF (100 mL) PPh₃ was added (7.1 g, 27.15 mmol) followed by MeOH (3 mL). The reaction mixture was cooled at 0° C. and DBAB (4.6 g, 20.36 mmol) was added to the reaction mixture portion wise and stirred at RT for 12 h. The mixture was then diluted with water (50 mL) and extracted with EtOAc (2×100 mL). The combined organic layer was washed with water (2×50 mL) and brine, dried over Na₂SO₄ and the solvent was evaporated under reduced pressure. The crude was purified by column chromatography over silica gel using 50% EA-Hex and 5-iodo-3-methoxypyridazine was isolated as white solid (3.0 g, 93% yield). ¹H NMR (400 MHz, DMSO-d₆): δ 7.8 (s, 1H), 7.4 (s, 1H), 3.7 (s, 3H).

Synthesis of 3-methoxy-5-((4-methoxybenzyl)thio)pyridazine 34.3: Intermediate 34.3 was synthesized from 5-iodo-3-methoxypyridazine 34.2 following a method analogous to that described in Method I using (4-methoxyphenyl)methanethiol. The crude was purified by column chromatography over silica gel using 20% EA-Hex to get 3-methoxy-5-((4-methoxybenzyl)thio)pyridazine (258 mg, 75% yield) as yellow gum. ¹H NMR (400 MHz, DMSO-d₆): δ 7.78 (d, J=1.6 Hz, 1H), 7.36 (d, J=8.3 Hz, 2H), 6.90 (d, J=8.4 Hz, 2H), 6.72 (d, J=1.6 Hz, 1H), 4.30 (s, 2H), 3.73 (s, 3H), 3.56 (s, 3H).

Synthesis of 6-methoxypyridazine-4-sulfonyl chloride 34.4: Sulfonyl chloride 34.4 was synthesized from Intermediate 34.3 following the procedure described in Method T. The crude sulfonyl chloride was used immediately in the forwarding step without further purification.

Synthesis of 6-methoxy-N-(2,2,2-trifluoro-1-(4-fluorophenyl)ethyl)pyridazine-4-sulfonamide 34.5: Intermediate 34.5 was synthesized from sulfonyl chloride 34.4 following the protocol as described in Method O using 2,2,2-trifluoro-1-(4-fluorophenyl)ethan-1-amine hydrochloride. The crude was purified by column chromatography over silica gel using 35% EA-hexane and intermediate 34.5 was isolated as yellow gum (60 mg, 17% yield). ¹H NMR (400 MHz, DMSO-d₆): δ 9.98 (s, 1H), 7.96-7.90 (m, 1H), 7.58-7.49 (m, 2H), 7.19 (t, J=8.7 Hz, 2H), 7.03 (s, 1H), 5.67-5.30 (m, 1H), 3.58 (s, 3H).

Synthesis of N-ethyl-6-methoxy-N-(2,2,2-trifluoro-1-(4-fluorophenyl)ethyl)pyridazine-4-sulfonamide (Example 70): Ethylation of intermediate 34.5 was preformed following the protocol as described in Method D using Cs₂CO₃ at 60° C. The compound was purified by column chromatography over silica gel using 35% ethyl acetate in hexane and Example 70 was isolated as colourless sticky gum (12 mg, 25% yield, 98.42% purity). LCMS: m/z found 394.0 [M+H]⁺, rt=2.84 min (Method 9) [Xbridge C18 column (3.5 μm, 50×3 mm)]; ¹H NMR (400 MHz, DMSO-d₆): δ 8.30 (s, 1H), 7.55 (t, J=5.7 Hz, 2H), 7.42 (s, 1H), 7.30 (t, J=8.6 Hz, 2H), 6.09 (q, J=8.6 Hz, 1H), 3.69 (s, 3H), 3.52-3.35 (m, 2H), 0.98 (t, J=7.0 Hz, 3H).

### Example 71 to Example 73

### 5-Cyano-N-ethyl-N-(2,2,2-trifluoro-1-(3-methoxyphenyl)ethyl)pyridine-3-sulfonamide (Example 71)

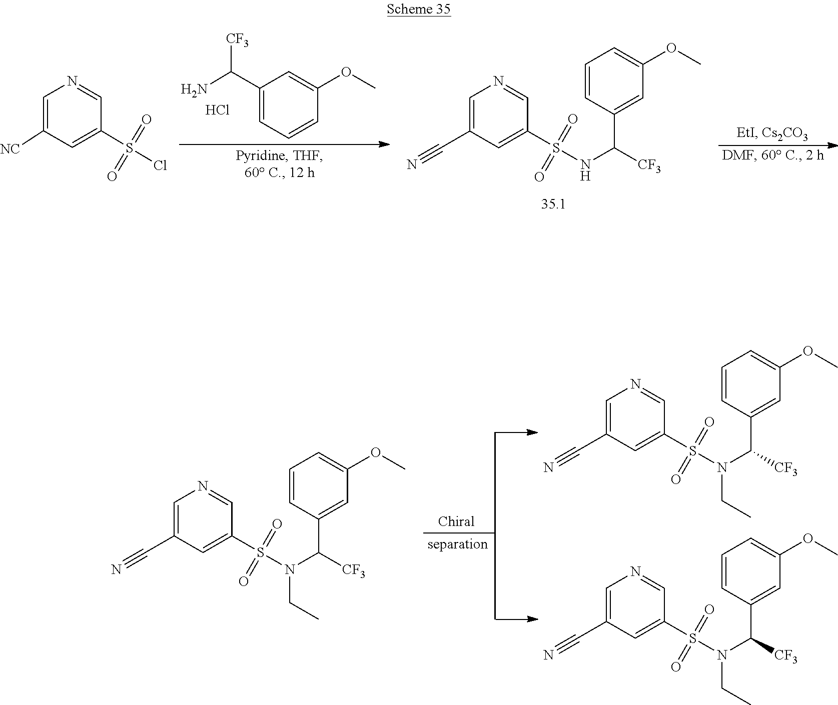

Synthesis of 5-cyano-N-(2,2,2-trifluoro-1-(3-methoxyphenyl)ethyl)pyridine-3-sulfonamide 35.1: Intermediate 35.1 was synthesized from 5-cyanopyridine-3-sulfonyl chloride following the protocol as described in Method O using 2,2,2-trifluoro-1-(3-methoxyphenyl)ethan-1-amine hydrochloride at 60° C. The compound was purified by column chromatography over silica gel using 25% ethyl acetate in hexane and isolated as colourless gum (420 mg, 45% yield). ¹H NMR (400 MHz, DMSO-d₆): δ 9.70 (s, 1H), 9.09 (s, 1H), 8.99 (s, 1H), 8.38 (s, 1H), 7.14 (t, J=7.8 Hz, 1H), 6.98-6.89 (m, 2H), 6.81 (d, J=8.1 Hz, 1H), 5.50-5.20 (m, 1H), 3.67 (s, 3H).

Synthesis of 5-cyano-N-ethyl-N-(2,2,2-trifluoro-1-(3-methoxyphenyl)ethyl)pyridine-3-sulfonamide (Example 71): The ethylation of intermediate 6.1 was performed following the protocol as described in Method D at 60° C. The crude was purified by column chromatography over silica gel using 10% ethyl acetate in hexane and the desired compound Example 71 was isolated as off white solid (36 mg, 26% yield, 98.76% purity). LCMS: m/z found 400.1 [M+H]⁺, rt=9.36 min (Method 6) [Waters Xbridge C18 column (5 μm, 50×4.6 mm)]; ¹H NMR (400 MHz, Chloroform-d): δ 9.22 (d, J=2.1 Hz, 1H), 9.02 (d, J=1.8 Hz, 1H), 8.31 (t, J=2.0 Hz, 1H), 7.33 (t, J=8.0 Hz, 1H), 7.03 (d, J=7.7 Hz, 1H), 7.01-6.92 (m, 2H), 5.74 (q, J=8.4 Hz, 1H), 3.81 (s, 3H), 3.48-3.34 (m, 1H), 3.34-3.20 (m, 1H), 0.98 (t, J=7.0 Hz, 3H).

Chiral separation of racemic Example 71 provided both the enantiomers as mentioned below. Chiral separation method: Chiral separation was done on Agilent 1200 series instrument. Column name: CHIRALPAK IA (250×20 mm) 5u. Operating at ambient temperature and flow rate is 18.0 mL/min. Mobile phase was a mixture of 80% Hexane and 20% ETOH, held this isocratic mixture run up to 20 min with wavelength of 224 nm.

Example 72: (R)-5-Cyano-N-ethyl-N-(2,2,2-trifluoro-1-(3-methoxyphenyl)ethyl)pyridine-3-sulfonamide Off white solid (18 mg, 99.87% purity). LCMS: m/z found 400.1 [M+H]⁺, rt=3.11 min (Method 2) [Acquity BEH C18 column (1.7 μm, 50×2.1 mm)]; ¹H NMR (400 MHz, Chloroform-d): δ 9.22 (s, 1H), 9.02 (s, 1H), 8.31 (s, 1H), 7.33 (t, J=8.0 Hz, 1H), 7.04 (d, J=7.7 Hz, 1H), 7.01-6.91 (m, 2H), 5.74 (q, J=8.4 Hz, 1H), 3.81 (s, 3H), 3.59-3.06 (m, 2H), 0.98 (t, J=7.0 Hz, 3H).

Example 73: (S)-5-Cyano-N-ethyl-N-(2,2,2-trifluoro-1-(3-methoxyphenyl)ethyl)pyridine-3-sulfonamide Off white solid (26 mg, 99.82% purity). LCMS: m/z found 400.1 [M+H]⁺, rt=3.11 min (Method 2) [Acquity BEH C18 column (1.7 μm, 50×2.1 mm)]; ¹H NMR (400 MHz, Chloroform-d): δ 9.23 (s, 1H), 9.02 (s, 1H), 8.31 (s, 1H), 7.33 (t, J=7.9 Hz, 1H), 7.03 (d, J=7.6 Hz, 1H), 7.00-6.93 (m, 2H), 5.74 (q, J=8.2 Hz, 1H), 3.81 (s, 3H), 3.51-3.19 (m, 2H), 0.98 (t, J=7.0 Hz, 3H).

### Example 74 and Example 75

### N,2-dimethyl-N-(2,2,2-trifluoro-1-(4-fluorophenyl)ethyl)pyrimidine-5-sulfonamide (Example 74) and N-ethyl-2-methyl-N-(2,2,2-trifluoro-1-(4-fluorophenyl)ethyl)pyrimidine-5-sulfonamide (Example 75)

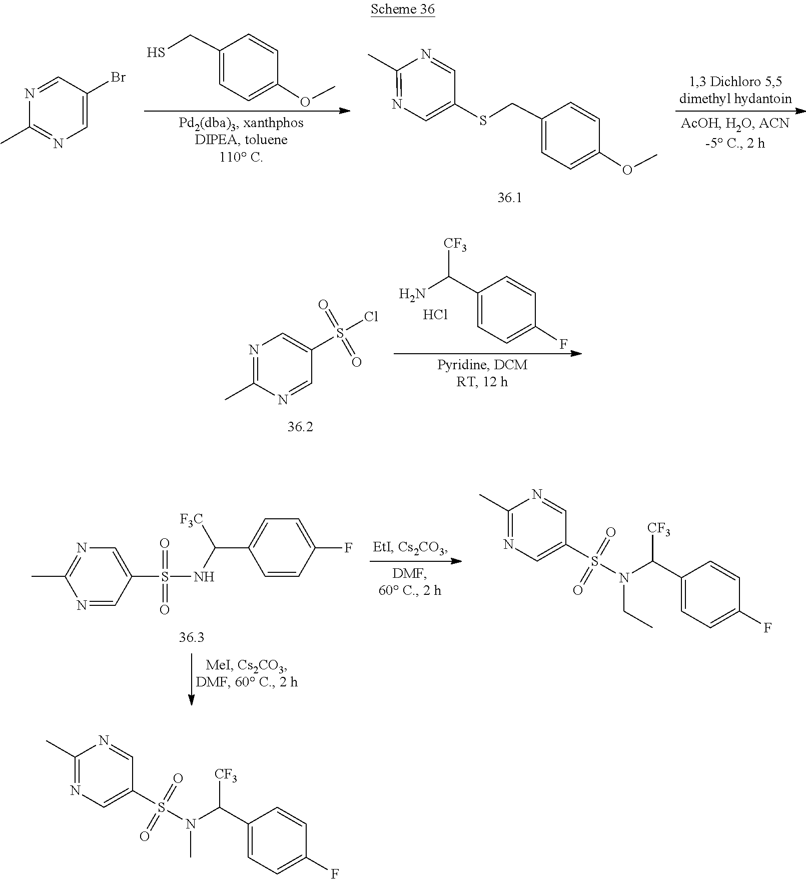

Synthesis of 5-((4-methoxybenzyl)thio)-2-methylpyrimidine 36.1: Intermediate 36.1 was synthesized from 5-bromo-2-methylpyrimidine following a method analogous to that described in Method I using (4-methoxyphenyl)-methanethiol. The crude was purified by column chromatography over silica gel using 20% ethyl acetate in hexane and isolated as brown sticky gum (1.6 g, 75% yield). ¹H NMR (400 MHz, DMSO-d₆): δ 8.58 (s, 2H), 7.21 (d, J=8.6 Hz, 2H), 6.85 (d, J=8.4 Hz, 2H), 4.21 (s, 2H), 3.71 (s, 3H), 2.56 (s, 3H).

Synthesis of 2-methylpyrimidine-5-sulfonyl chloride 36.2: Sulfonyl chloride 36.2 was synthesized from Intermediate 36.1 following the procedure described in Method T. The crude sulfonyl chloride was used immediately in the forwarding step without further purification.

Synthesis of 2-methyl-N-(2,2,2-trifluoro-1-(4-fluorophenyl)ethyl)pyrimidine-5-sulfonamide 36.3: Intermediate 36.3 was synthesized from sulfonyl chloride 36.2 following the protocol as described in Method O using 2,2,2-trifluoro-1-(4-fluorophenyl)ethan-1-amine hydrochloride in DCM. The compound was purified by column chromatography over silica gel using 35% ethyl acetate in hexane and isolated as yellow gum (300 mg, 25% yield). ¹H NMR (400 MHz, DMSO-d₆): δ 9.72 (d, J=9.6 Hz, 1H), 8.81 (s, 2H), 7.51-7.38 (m, 2H), 7.12 (t, J=8.3 Hz, 2H), 5.62-5.33 (m, 1H), 2.60 (s, 3H).

Synthesis of N-ethyl-2-methyl-N-(2,2,2-trifluoro-1-(4-fluorophenyl)ethyl)pyrimidine-5-sulfonamide (Example 74): The ethylation of intermediate 36.3 was performed following the protocol as described in Method D at 60° C. The crude was purified by column chromatography over silica gel using 10% ethyl acetate in hexane and the desired compound was isolated as colorless gum (20 mg, 19% yield, 98.49% purity). LCMS: m/z found 378.12 [M+H]⁺, rt=3.08 min (Method 2) [Waters Acquity BEH C8 column (1.7 μm, 50×2.1 mm)]; ¹H NMR (400 MHz, Chloroform-d): δ 9.01 (s, 2H), 7.49 (dd, J=5.1, 8.6 Hz, 2H), 7.17-7.07 (m, 2H), 5.81 (q, J=8.3 Hz, 1H), 3.45-3.31 (m, 1H), 3.25-3.11 (m, 1H), 2.83 (s, 3H), 0.93 (t, J=7.0 Hz, 3H).

Synthesis of N,2-dimethyl-N-(2,2,2-trifluoro-1-(4-fluorophenyl)ethyl)pyrimidine-5-sulfonamide (Example 75): The methylation of intermediate 7.3 was preformed following the protocol as described in Method D at 60° C. The crude was purified by column chromatography over silica gel using 10% ethyl acetate in hexane and the desired compound was isolated as white solid (30 mg, 29% yield, 97.66% purity). LCMS: m/z found 364.07 [M+H]⁺, rt=2.99 min (Method 2) [Waters Acquity BEH C8 column (1.7 μm, 50×2.1 mm)]; ¹H NMR (400 MHz, Chloroform-d): δ 9.00 (s, 2H), 7.44 (dd, J=5.1, 8.6 Hz, 2H), 7.18-7.07 (m, 2H), 5.87 (q, J=8.2 Hz, 1H), 2.84 (s, 3H), 2.73 (s, 3H).

### Example 76 and Example 77

### (S)—N-ethyl-2-methyl-N-(2,2,2-trifluoro-1-(4-fluorophenyl)ethyl)pyrimidine-5-sulfonamide (Example 76) and (R)—N-ethyl-2-methyl-N-(2,2,2-trifluoro-1-(4-fluorophenyl)ethyl)pyrimidine-5-sulfonamide (Example 77

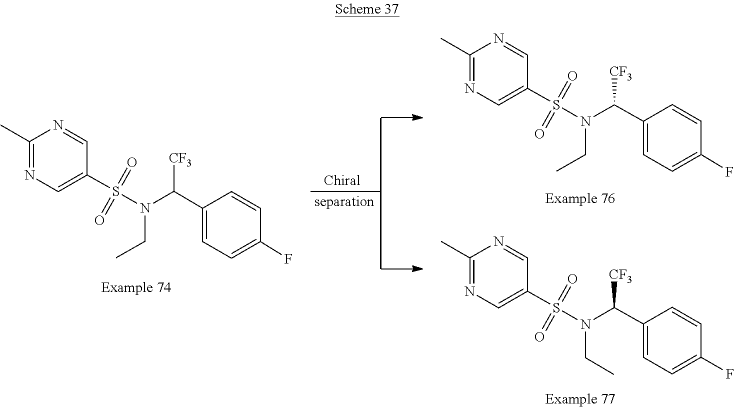

Chiral separation of racemic Example 75 provided both the enantiomers as mentioned below. Chiral separation method: Chiral separation was done on Agilent 1200 series instrument. Column name: CHIRALPAK IG (250×21 mm) 5u. Operating at ambient temperature and flow rate is 21.0 mL/min. Mobile phase was mixture of 70% Hexane and 30% ETOH, held this isocratic mixture run up to 25 min with wavelength of 228 nm.

Example 76: Colorless gum (28 mg, 99.88% purity). LCMS: m/z found 378.15 [M+H]⁺, rt=3.09 min (Method 2) [Acquity BEH C18 column (1.7 μm, 50×2.1 mm)]; ¹H NMR (400 MHz, Chloroform-d): δ 9.01 (s, 2H), 7.49 (dd, J=5.2, 8.4 Hz, 2H), 7.12 (t, J=8.4 Hz, 2H), 5.81 (q, J=8.3 Hz, 1H), 3.45-3.31 (m, 1H), 3.26-3.11 (m, 1H), 2.83 (s, 3H), 0.94 (t, J=7.0 Hz, 3H).

Example 77: Colorless gum (24 mg, 99.54% purity). LCMS: m/z found 378.14 [M+H]⁺, rt=3.09 min (Method 2) [Acquity BEH C18 column (1.7 μm, 50×2.1 mm)]; ¹H NMR (400 MHz, Chloroform-d): δ 9.01 (s, 2H), 7.49 (dd, J=5.1, 8.7 Hz, 2H), 7.12 (t, J=8.4 Hz, 2H), 5.81 (q, J=8.3 Hz, 1H), 3.60-3.02 (m, 2H), 2.83 (s, 3H), 0.94 (t, J=7.0 Hz, 3H).

### Example 78 and Example 79

### (R)—N,2-dimethyl-N-(2,2,2-trifluoro-1-(4-fluorophenyl)ethyl)pyrimidine-5-sulfonamide (Example 78 (EN-1)) and (S)—N,2-dimethyl-N-(2,2,2-trifluoro-1-(4-fluorophenyl)ethyl)pyrimidine-5-sulfonamide (Example 79 (EN-2

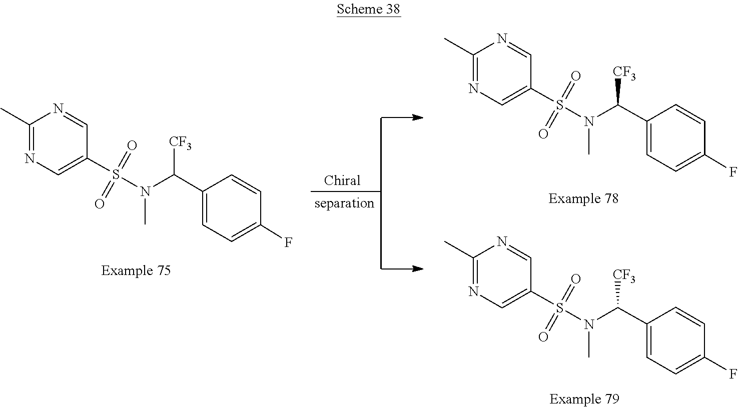

Chiral separation of racemic Example 75 provided both the enantiomers as described below. Chiral separation method: chiral separation was performed on an Agilent 1200 series instrument. Column name: CHIRALPAK IG (250×21 mm) 5u, operating at ambient temperature and at a flow rate of 21.0 mL/min. Mobile phase was a mixture of 70% Hexane and 30% EtOH, this isocratic mixture run up to 30 min at a wavelength of 230 nm.

Example 78: White solid (21 mg, 99.10% purity). LCMS: m/z found 364.0 [M+H]⁺, rt=1.89 min (Method 3) [YMC Triart C18 column (3 μm, 33×2.1 mm)]; ¹H NMR (400 MHz, Chloroform-d): δ 9.00 (s, 2H), 7.44 (dd, J=5.1, 8.6 Hz, 2H), 7.12 (t, J=8.5 Hz, 2H), 5.87 (q, J=8.2 Hz, 1H), 2.84 (s, 3H), 2.73 (s, 3H).

Example 79: White solid (22 mg, 98.33% purity). LCMS: m/z found 364.0 [M+H]⁺, rt=1.89 min (Method 3) [YMC Triart C18 column (3 μm, 33×2.1 mm)]; ¹H NMR (400 MHz, Chloroform-d): δ 9.00 (s, 2H), 7.44 (dd, J=5.1, 8.6 Hz, 2H), 7.12 (t, J=8.5 Hz, 2H), 5.86 (q, J=8.2 Hz, 1H), 2.84 (s, 3H), 2.73 (s, 3H).

### Example 80 and Example 81

### 5-Cyano-N-ethyl-N-(2,2,2-trifluoro-1-(4-fluorophenyl)ethyl)thiazole-2-sulfonamide (Example 80) and N-ethyl-N-(2,2,2-trifluoro-1-(4-fluorophenyl)ethyl)thiazole-2-sulfonamide (Example 81)

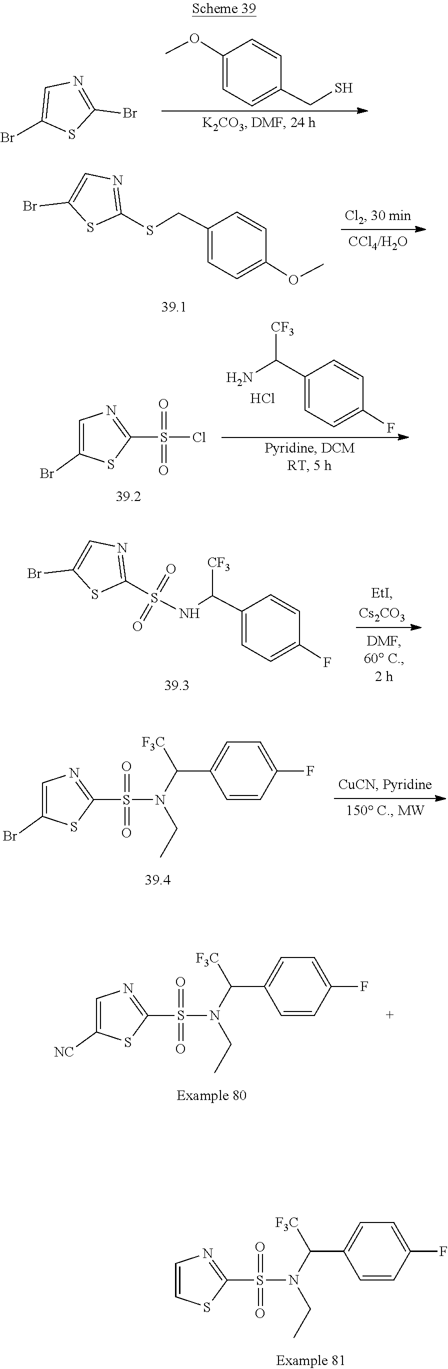

Synthesis of 5-bromo-2-((4-methoxybenzyl)thio)thiazole 39.1: To a stirred solution of 2,5-dibromothiazole (1 g, 4.12 mmol) in DMF (5 mL) K₂CO₃ (0.58 g, 4.2 mmol) was added. The resulting mixture was stirred for 15 min and the solution of (4-methoxyphenyl)methanethiol (650 mg, 4.2 mmol) in DMF (5 mL) was added to it. The reaction was stirred at RT for 12 h. The reaction mixture was then poured into ice water and extracted with ethyl acetate (3×25 mL). The combined organic layer was washed with brine (25 mL), dried over Na₂SO₄ and concentrated under reduced pressure. The crude was purified by combi-flash column chromatography using 5% ethyl acetate in hexane as eluent to afford desire product 39.1 as a yellow gum (900 mg, 69% yield). LCMS: m/z found 318.0 [M+H+2]⁺.

Synthesis of 5-bromothiazole-2-sulfonyl chloride 39.2: 5-bromo-2-((4-methoxybenzyl)thio)thiazole (0.5 g, 1.58 mmol) was dissolved in CCl₄ (20 mL) and H₂O (2 mL) was added to it. Cl₂ gas was purged in the reaction for 30 min at 0° C. N₂ was flushed to remove excess Cl₂ gas. The mixture was then extracted with DCM (2×25 mL) and the combined organic layers were washed with water (20 mL), dried over Na₂SO₄ and concentrated to furnish the desire product which was used directly in the following step.

Synthesis of 5-bromo-N-(2,2,2-trifluoro-1-(4-fluorophenyl)ethyl)thiazole-2-sulfonamide 39.3: Intermediate 39.3 was synthesized from sulfonyl chloride 39.2 following the protocol as described in Method O using 2,2,2-trifluoro-1-(4-fluorophenyl)ethan-1-amine hydrochloride in DCM. The compound was purified by column chromatography over silica gel using 35% ethyl acetate in hexane and isolated as a yellow gum (150 mg, 31% yield). LCMS: m/z found 420.85 [M+H+2]⁺.

Synthesis of 5-bromo-N-ethyl-N-(2,2,2-trifluoro-1-(4-fluorophenyl)ethyl)thiazole-2-sulfonamide 39.4: The methylation of intermediate 39.3 was preformed following the protocol as described in Method D at 60° C. The crude residue was purified by column chromatography over silica gel using 10% ethyl acetate in hexane and the desired compound was isolated as a yellow gum (80 mg, 50% yield). LCMS: m/z found 447.22 [M+H]⁺.

Synthesis of 5-cyano-N-ethyl-N-(2,2,2-trifluoro-1-(4-fluorophenyl)ethyl)thiazole-2-sulfonamide (Example 80) and N-ethyl-N-(2,2,2-trifluoro-1-(4-fluorophenyl)ethyl)thiazole-2-sulfonamide (Example 81): To a solution of intermediate 39.4 (135 mg, 0.30 mmol) in pyridine (2 mL) CuCN (189 mg, 2.11 mmol) was added and the reaction mixture was heated at 150° C. in a microwave oven for 1 h. The reaction mixture was filtered and concentrated under reduced pressure. The crude residue was diluted with water (10 mL) and extracted with ethyl acetate (2×15 mL). The combined organic layers were washed with water (10 mL), dried over Na₂SO₄ and concentrated under reduced pressure. The crude was purified by combiflash column chromatography using 10% ethyl acetate in hexane as eluent to afford both Example 80 and Example 81.

5-cyano-N-ethyl-N-(2,2,2-trifluoro-1-(4-fluorophenyl)ethyl)thiazole-2-sulfonamide (Example 80): White solid (19 mg, 17% yield, 99.46% purity). LCMS: m/z found 394.2 [M+H]⁺, rt=3.66 min (Method 4) [Xbridge C8 column (5 μm, 50×4.6 mm)]; ¹H NMR (400 MHz, Chloroform-d): δ 8.33 (d, J=0.9 Hz, 1H), 7.50 (t, J=6.9 Hz, 2H), 7.11 (t, J=8.1 Hz, 2H), 5.74 (q, J=8.0 Hz, 1H), 3.59-3.41 (m, 2H), 1.01 (t, J=6.9 Hz, 3H).

N-ethyl-N-(2,2,2-trifluoro-1-(4-fluorophenyl)ethyl)thiazole-2-sulfonamide (Example 81): Colorless sticky gum (15 mg, 14% yield, 99.02% purity). LCMS: m/z found 369.11 [M+H]1, rt=2.56 min (Method 2) [Acquity BEH C8 column (1.7 μm, 50×2.1 mm)]; ¹H NMR (400 MHz, Chloroform-d): δ 7.95 (d, J=3.0 Hz, 1H), 7.61 (d, J=3.1 Hz, 1H), 7.47 (dd, J=5.1, 8.5 Hz, 2H), 7.06 (t, J=8.6 Hz, 2H), 5.79 (q, J=8.0 Hz, 1H), 3.52-3.35 (m, 2H), 1.04 (t, J=7.0 Hz, 3H).

### Example 82

### 2-Cyano-N-ethyl-N-(2,2,2-trifluoro-1-(4-fluorophenyl)ethyl)thiazole-5-sulfonamide

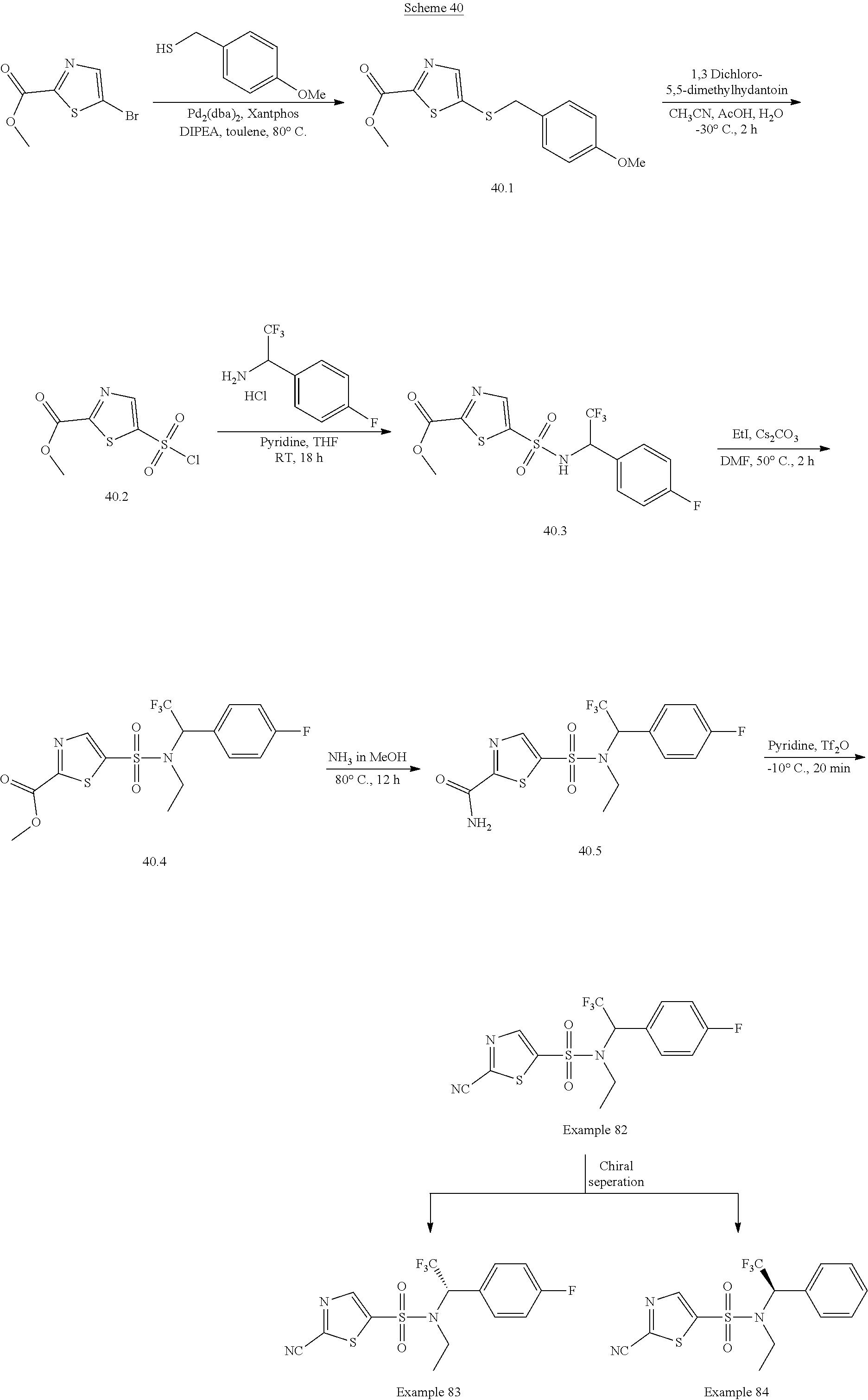

Synthesis of methyl 5-((4-methoxybenzyl)thio)thiazole-2-carboxylate 40.1: Intermediate 40.1 was synthesized from methyl 5-bromothiazole-2-carboxylate following a method analogous to that described in Method I using (4-methoxyphenyl)methanethiol. The crude residue was purified by column chromatography over silica gel using 20% ethyl acetate in hexane and isolated as a yellow oil (800 mg, 92% yield). LCMS: m/z found 296.35 [M+H]⁺, rt=1.91 min (Method 5) [Waters Acquity BEH C18 column (1.7 μm, 50×2.1 mm)]; ¹H NMR (400 MHz, DMSO-d₆): δ 7.92 (s, 1H), 7.21 (d, J=8.4 Hz, 2H), 6.87 (d, J=8.4 Hz, 2H), 4.22 (s, 2H), 3.88 (s, 3H), 3.72 (s, 3H).

Synthesis of methyl 5-(chlorosulfonyl)thiazole-2-carboxylate 40.2: Sulfonyl chloride 40.2 was synthesized from Intermediate 40.1 following the procedure described in Method T. The crude sulfonyl chloride was purified by column chromatography over silica gel and the compound 40.2 was isolated as white solid. ¹H NMR (400 MHz, DMSO-d₆) δ 7.98 (s, 1H), 3.90 (s, 3H).

Synthesis of methyl 5-(N-(2,2,2-trifluoro-1-(4-fluorophenyl)ethyl)sulfamoyl)thiazole-2-carboxylate 40.3: Intermediate 40.3 was synthesized from sulfonyl chloride 40.2 following the protocol as described in Method O using 2,2,2-trifluoro-1-(4-fluorophenyl)ethan-1-amine hydrochloride. The compound was purified by column chromatography over silica gel using 30% ethyl acetate in hexane and compound 40.3 (400 mg, 98% yield) was isolated as white solid LCMS: m/z found 399.2 [M+H]⁺, rt=2.10 min (Method 12) [YMC Triart C18 column (3 μm, 33×2.1 mm)], ¹H NMR (400 MHz, DMSO-d₆): δ 8.29 (s, 1H), 7.51 (t, J=5.2 Hz, 3H), 7.16 (t, J=8.7 Hz, 2H), 5.72-5.46 (m, 1H), 3.91 (s, 3H).

Synthesis of methyl 5-(N-ethyl-N-(2,2,2-trifluoro-1-(4-fluorophenyl)ethyl)sulfamoyl)thiazole-2-carboxylate 40.4: The ethylation of intermediate 40.3 was performed following the protocol as described in Method D at 60° C. in DMSO. The crude was purified by column chromatography over silica gel using 10% ethyl acetate in hexane and the desired compound 40.4 (90 mg, 56% yield) was isolated as colourless liquid. ¹H NMR (400 MHz, Chloroform-d): δ 7.94 (d, J=2.4 Hz, 1H), 7.31 (t, J=5.2 Hz, 2H), 7.02 (t, J=8.8 Hz, 2H), 5.18 (q, J=6.4 Hz, 1H), 3.81 (s, 3H), 3.24-3.13 (m, 2H), 0.98 (t, J=6.8 Hz, 3H).

Synthesis of 5-(N-ethyl-N-(2,2,2-trifluoro-1-(4-fluorophenyl)ethyl)sulfamoyl)thiazole-2-carboxamide 40.5: Intermediate 40.4 (200 mg, 0.47 mmol) was taken in NH₃/MeOH (4 mL) in a sealed tube and then heated at 80° C. for 12 h. The volatiles were evaporated under reduced pressure and the crude was purified by column chromatography over silica gel using 70% ethyl acetate in hexane to afford 5-(N-ethyl-N-(2,2,2-trifluoro-1-(4-fluorophenyl)ethyl)sulfamoyl)thiazole-2-carboxamide 40.5 (95 mg, 49% yield) as a white solid. LCMS: m/z found 412.2 [M+H]⁺, rt=1.95 min (Method 25) [Waters Acquity BEH C18 column (1.7 μm, 50×2.1 mm)]; ¹H NMR (400 MHz, DMSO-d₆): δ 8.56 (s, 1H), 8.48 (s, 1H), 8.18 (s, 1H), 7.54-7.43 (m, 2H), 7.27 (t, J=8.6 Hz, 2H), 6.14-5.98 (m, 1H), 3.48-3.32 (m, 2H), 1.03 (t, J=7.0 Hz, 3H).

Synthesis of 2-cyano-N-ethyl-N-(2,2,2-trifluoro-1-(4-fluorophenyl)ethyl)thiazole-5-sulfonamide (Example 82): To a stirred solution of carboxamide 11.5 (120 mg, 0.29 mmol) in pyridine (7 mL) TFAA (0.16 mL, 1.17 mmol) was added at −10° C. and the reaction was stirred at this temperature for 20 min. The reaction mixture was diluted with water (10 mL) and extracted with ethyl acetate (2×15 mL). The combined organic layers were washed with brine (15 mL), dried over Na₂SO₄ and the solvent was evaporated under reduced pressure. The crude was purified by column chromatography over silica-gel using 20% ethyl acetate in hexane. The desired compound (Example 82) (95 mg, 83% yield) was isolated as a sticky gum. LCMS: m/z found 392.2 [M−H], rt=3.69 min (Method 4) [Xbridge C18 column (5 μm, 50×4.6 mm))]; ¹H NMR (400 MHz, DMSO-d₆): δ 8.79 (s, 1H), 7.52-7.42 (m, 2H), 7.29 (t, J=8.6 Hz, 2H), 6.12 (q, J=8.8 Hz, 1H), 3.48-3.34 (m, 2H), 1.03 (t, J=6.8 Hz, 3H).

### Example 83 and Example 84

### (S)-2-Cyano-N-ethyl-N-(2,2,2-trifluoro-1-(4-fluorophenyl)ethyl)thiazole-5-sulfonamide (Example 83 EN-1)) and (R)-2-cyano-N-ethyl-N-(2,2,2-trifluoro-1-phenylethyl)thiazole-5-sulfonamide (Example 84 (EN-2

Chiral separation of racemic Example 82 provided both the enantiomers as described below. Chiral separation method: Chiral separation was done on an Agilent 1200 series instrument. Column name: CHIRALCEL OJ-H (250×21 mm), 5μ, operating at ambient temperature and flow rate of 21.0 mL/min. Mobile phase was 0.1% of isopropylamine in a mixture of 90% hexane and 10% ethanol, this isocratic mixture held run up to 24 min at a wavelength of 250 nm.

Example 83: Sticky gum (99.94% purity). LCMS: m/z found 392.2 [M−H], rt=3.69 min (Method 4) [Xbridge C18 column (5 μm, 50×4.6 mm))]; ¹H NMR (400 MHz, DMSO-d₆): δ 8.79 (s, 1H), 7.52-7.42 (m, 2H), 7.29 (t, J=8.6 Hz, 2H), 6.12 (q, J=8.8 Hz, 1H), 3.48-3.34 (m, 2H), 1.03 (t, J=6.8 Hz, 3H).

Example 84: Sticky gum (99.94% purity). LCMS: m/z found 392.2 [M−H], rt=3.69 min (Method 4) [Xbridge C18 column (5 μm, 50×4.6 mm))]; ¹H NMR (400 MHz, DMSO-d₆): δ 8.78 (s, 1H), 7.51-7.43 (m, 2H), 7.29 (t, J=8.7 Hz, 2H), 6.12 (t, J=8.1 Hz, 1H), 3.48-3.31 (m, 2H), 1.03 (d, J=6.8 Hz, 3H).

### Example 85

### 5-Cyano-N-ethyl-N-(2,2,2-trifluoro-1-(3-(trifluoromethyl)phenyl)ethyl)pyridine-3-sulfonamide

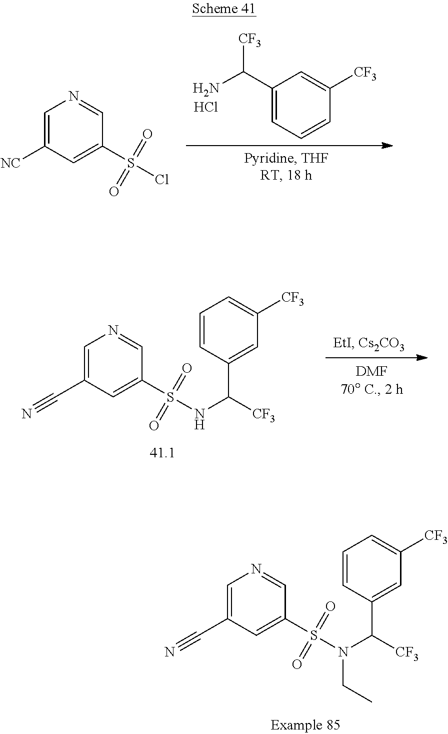

Synthesis of 5-cyano-N-(2,2,2-trifluoro-1-(3-(trifluoromethyl)phenyl)ethyl)pyridine-3-sulfonamide 41.1: Intermediate 41.1 was synthesized from 5-cyanopyridine-3-sulfonyl chloride following the protocol as described in Method O using 2,2,2-trifluoro-1-(3-(trifluoromethyl)phenyl)ethan-1-amine hydrochloride. The compound was purified by column chromatography over silica gel using 20% ethyl acetate in hexane and compound 41.1 (200 mg, 44% yield) was isolated as yellow gum. LCMS: m/z found 409.99 [M+H]⁺, rt=1.93 min (Method 5) [Waters Acquity BEH C18 column (1.7 μm, 50×2.1 mm))]; ¹H NMR (400 MHz, DMSO-d₆): δ 10.09-9.83 (m, 1H), 9.10 (d, J=1.8 Hz, 1H), 8.97 (d, J=2.0 Hz, 1H), 8.45 (t, J=2.0 Hz, 1H), 7.78 (s, 1H), 7.74 (d, J=7.8 Hz, 1H), 7.67 (d, J=7.8 Hz, 1H), 7.54 (t, J=7.8 Hz, 1H), 5.75-5.59 (m, 1H).

Synthesis of 5-cyano-N-ethyl-N-(2,2,2-trifluoro-1-(3-(trifluoromethyl)phenyl)ethyl)pyridine-3-sulfonamide: The ethylation of intermediate 41.1 was performed following the protocol as described in Method D at 60° C. The crude was purified by column chromatography over silica gel using 20% ethyl acetate in hexane and the desired compound Example 85 (30 mg, 28% yield, 96.16% purity) was isolated as a light yellow gum. LCMS: m/z found 438.1 [M+H]⁺, rt=9.61 min (Method 6) [Xbridge C8 column (5 μm, 50×4.6 mm)]; ¹H NMR (400 MHz, Chloroform-d): δ 9.25 (d, J=2.2 Hz, 1H), 9.06 (d, J=1.9 Hz, 1H), 8.38 (t, J=2.1 Hz, 1H), 7.81-7.66 (m, 3H), 7.60 (t, J=7.8 Hz, 1H), 5.86 (q, J=8.6 Hz, 1H), 3.51-3.36 (m, 1H), 3.29-3.15 (m, 1H), 0.96 (t, J=7.1 Hz, 3H).

### Example 86

### 5-cyano-N-ethyl-N-(2,2,2-trifluoro-1-(o-tolyl)ethyl)pyridine-3-sulfonamide

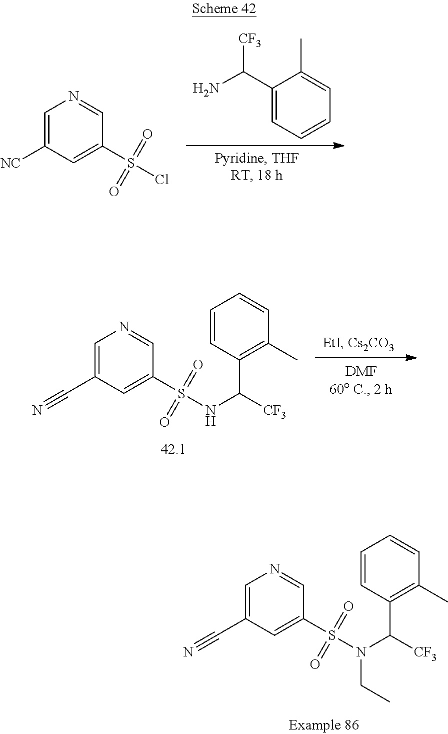

Synthesis of 5-cyano-N-(2,2,2-trifluoro-1-(o-tolyl)ethyl)pyridine-3-sulfonamide 42.1: Intermediate 42.1 was synthesized from 5-cyanopyridine-3-sulfonyl chloride following the protocol as described in Method O using 2,2,2-trifluoro-1-(o-tolyl)ethan-1-amine. The compound was purified by column chromatography over silica gel using 20% ethyl acetate in hexane and compound 42.1 (250 mg, 63% yield) was isolated as a yellow gum. LCMS: m/z found 356.24 [M+H]⁺, rt=1.85 min (Method 3) [YMC Triart C18 column (3 μm, 33×2.1 mm)]; ¹H NMR (400 MHz, DMSO-d₆): δ 9.98-9.75 (m, 1H), 9.08 (d, J=1.8 Hz, 1H), 8.97 (d, J=2.1 Hz, 1H), 8.32 (t, J=2.0 Hz, 1H), 7.30 (d, J=7.6 Hz, 1H), 7.22-7.11 (m, 2H), 7.10-6.99 (m, 1H), 5.59-5.09 (m, 1H), 2.30 (s, 3H).

Synthesis of 5-cyano-N-ethyl-N-(2,2,2-trifluoro-1-(o-tolyl)ethyl)pyridine-3-sulfonamide (Example 86): The ethylation of intermediate 42.1 was preformed following the protocol as described in Method D at 60° C. The crude was purified by column chromatography over silica gel using 20% ethyl acetate in hexane and the desired compound Example 86 (130 mg, 60% yield, 97.66% purity) was isolated as a light yellow gum. LCMS: m/z found 384.16 [M+H]⁺, rt=3.18 min (Method 2) [Waters Acquity BEH C8 column (1.7 μm, 50×2.1 mm)], ¹H NMR (400 MHz, Chloroform-d): δ 9.22 (s, 1H), 9.02 (s, 1H), 8.30 (t, J=2.0 Hz, 1H), 7.42 (d, J=7.8 Hz, 1H), 7.37 (t, J=7.4 Hz, 1H), 7.30 (d, J=7.2 Hz, 1H), 7.25-7.20 (m, 1H), 6.00 (q, J=6.4 Hz, 1H), 3.40-3.22 (m, 2H), 2.54 (s, 3H), 0.70 (t, J=7.0 Hz, 3H).

### Example 87 and Example 88

### 5-(3,6-Dihydro-2H-pyran-4-yl)-N-ethyl-N-(2,2,2-trifluoro-1-(4-fluorophenyl)ethyl)thiophene-2-sulfonamide (Example 87) and N-ethyl-5-(tetrahydro-2H-pyran-4-yl)-N-(2,2,2-trifluoro-1-(4-fluorophenyl)ethyl)thiophene-2-sulfonamide (Example 88)

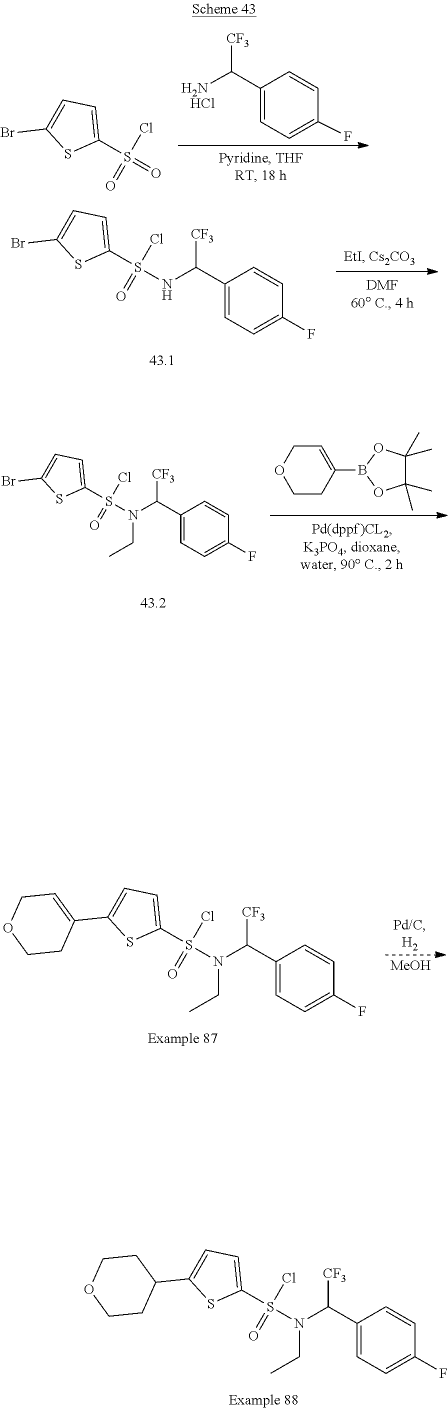

Synthesis of 5-bromo-N-(2,2,2-trifluoro-1-(4-fluorophenyl)ethyl)thiophene-2-sulfonamide 43.1: Intermediate 43.1 was synthesized from 5-bromothiophene-2-sulfonyl chloride following the protocol as described in Method O using 2,2,2-trifluoro-1-(4-fluorophenyl)ethan-1-amine hydrochloride. The compound was purified by column chromatography over silica gel using 25% ethyl acetate in hexane and isolated as a yellow oil (1.49 g, 48% yield). ¹H NMR (400 MHz, DMSO-d₆) δ: 9.68-9.65 (m, 1H) 7.55-7.47 (m, 2H), 7.26 (d, J=3.8 Hz, 1H), 7.19-7.10 (m, 3H), 5.40 (d, J=8.8 Hz, 1H).

5-bromo-N-ethyl-N-(2,2,2-trifluoro-1-(4-fluorophenyl)ethyl)thiophene-2-sulfonamide 43.2: The ethylation of intermediate 43.1 was preformed following the protocol as described in Method D at 60° C. The crude residue was purified by column chromatography over silica gel using 25% ethyl acetate in hexane and the desired compound intermediate 43.2 was isolated as a colorless sticky gum (780 mg, 76% yield). ¹H NMR (400 MHz, DMSO-d₆) δ: 7.66 (t, J=4.0 Hz, 1H) 7.50-7.35 (m, 3H), 7.26 (t, J=8.7 Hz, 2H), 6.00-5.90 (m, 1H), 3.10-3.50 (m, 2H), 0.99 (t, J=7.0 Hz, 3H).

Synthesis of 5-(3,6-dihydro-2H-pyran-4-yl)-N-ethyl-N-(2,2,2-trifluoro-1-(4-fluorophenyl)ethyl)thiophene-2-sulfonamide (Example 87): Suzuki coupling; Method V: 5-bromo-N-ethyl-N-(2,2,2-trifluoro-1-(4-fluorophenyl)ethyl)thiophene-2-sulfonamide (200 mg, 0.45 mmol) and 2-(3,6-dihydro-2H-pyran-4-yl)-4,4,5,5-tetramethyl-1,3,2-dioxaborolane (113 mg, 0.54 mmol) were dissolved in 1,4-dioxane (1 mL) and the solution was then degassed with argon. A solution of K₃PO₄ in water (1 mL) was added to it, followed by Pd(dppf)Cl₂ (33 mg, 0.045 mmol) under argon atmosphere. The reaction mixture was then heated at 90° C. for 2 h. After filtration through a celite bed, the filtrate was evaporated. The crude obtained was diluted with water (10 mL) and extracted with ethyl acetate (2×15 mL). The combined organic layers were washed with water (10 mL) and brine (10 mL). The solvent was evaporated under reduced pressure and the crude was further purified by column chromatography over silica gel using 40% ethyl acetate in hexane and the desired compound Example 87 was isolated as light orange sticky gum (150 mg, 74% yield, 95.59% purity). LCMS: m/z found 450.16 [M+H]⁺, rt=3.83 min (Method 2) [Waters Acquity BEH C8 column (1.7 μm, 50×2.1 mm)]; ¹H NMR (400 MHz, Chloroform-d): δ 7.48 (d, J=4.0 Hz, 1H), 7.40 (dd, J=5.2, 8.6 Hz, 2H), 7.06 (t, J=8.6 Hz, 2H), 6.91 (d, J=3.9 Hz, 1H), 6.27-6.21 (m, 1H), 5.75 (q, J=8.5 Hz, 1H), 4.30 (q, J=2.7 Hz, 2H), 3.91 (t, J=5.4 Hz, 2H), 3.42-3.28 (m, 1H), 3.24-3.10 (m, 1H), 2.52-2.45 (m, 2H), 1.01 (t, J=7.1 Hz, 3H).

Synthesis of N-ethyl-5-(tetrahydro-2H-pyran-4-yl)-N-(2,2,2-trifluoro-1-(4-fluorophenyl)ethyl)thiophene-2-sulfonamide (Example 88): Hydrogenation; Method U: compound Example 87 (200 mg, 0.445 mmol) was dissolved in EtOH (5 mL) and degassed with nitrogen. Pd (10% on C) (40 mg) was added to it and the reaction mixture was stirred under hydrogen atmosphere (balloon pressure) at RT overnight. The reaction mixture was filtered and the filtrate was evaporated under reduced pressure. The crude was then purified by reverse-phase preparative HPLC and the compound was isolated as a light brown solid (40 mg, 20% yield, 96.27% purity). Preparative HPLC was done on a Waters auto purification instrument. Column: KINETEX EVO C18 (250×21 mm, 5μ) operating at ambient temperature and flow rate of 16.0 ml/min. Mobile phase: A=10 mM NH₄OAC in water, B=Acetonitrile; Gradient Profile: Mobile phase initial composition of 80% A and 20% B, then to 45% A and 55% B in 3 min, then to 40% A and 60% B in 30 min, then to 5% A and 95% B in 31 min, held this composition up to 34 min. For column washing, then returned to initial composition in 35 min and held for 38 mins. LCMS: m/z found 451.8 [M+H]⁺, rt=3.83 min (Method 4) [Waters Xbridge C18 column (5 μm, 50×4.6 mm)]; ¹H NMR (400 MHz, Chloroform-d): δ 7.48 (d, J=3.8 Hz, 1H), 7.37 (dd, J=5.1, 8.5 Hz, 2H), 7.04 (t, J=8.6 Hz, 2H), 6.80 (d, J=3.8 Hz, 1H), 5.72 (q, J=8.5 Hz, 1H), 4.10-4.01 (m, 2H), 3.57-3.45 (m, 2H), 3.42-3.28 (m, 1H), 3.23-3.00 (m, 2H), 1.97-1.88 (m, 2H), 1.86-1.71 (m, 2H), 1.02 (t, J=7.0 Hz, 3H).

### Example 89 to Example 91

### N-ethyl-5-morpholino-N-(2,2,2-trifluoro-1-(4-fluorophenyl)ethyl)thiophene-2-sulfonamide (Example 89), (R)—N-ethyl-5-morpholino-N-(2,2,2-trifluoro-1-(4-fluorophenyl)ethyl)thiophene-2-sulfonamide (Example 90 (EN-1)) and (S)—N-ethyl-5-morpholino-N-(2,2,2-trifluoro-1-(4-fluorophenyl)ethyl)thiophene-2-sulfonamide (Example 91 (EN-2

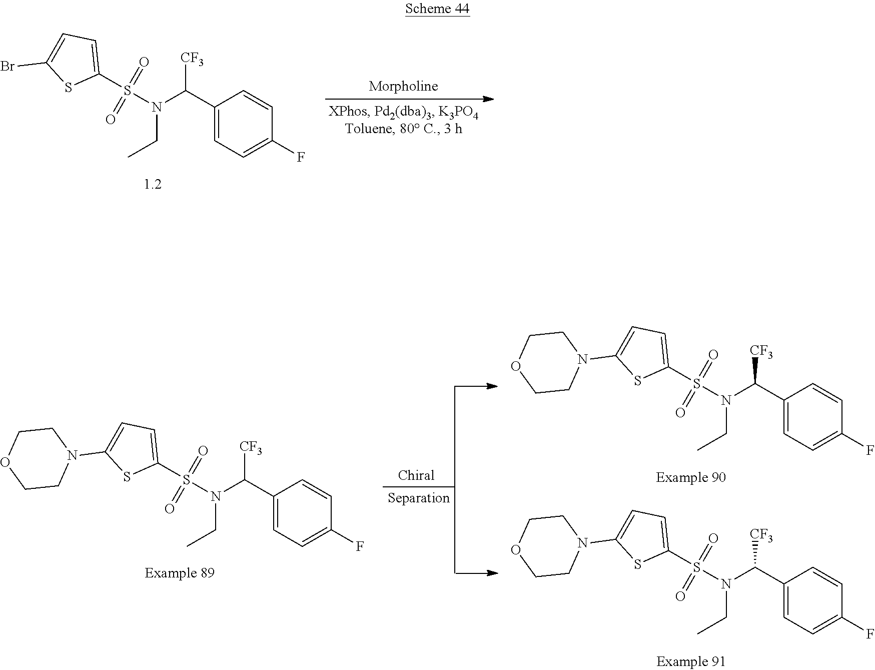

N-ethyl-5-morpholino-N-(2,2,2-trifluoro-1-(4-fluorophenyl)ethyl)thiophene-2-sulfonamide (Example 89): Buchwald coupling; Method W: 5-bromo-N-ethyl-N-(2,2,2-trifluoro-1-(4-fluorophenyl)ethyl)thiophene-2-sulfonamide 1.2 (200 mg, 0.45 mmol) was dissolved in toluene (1 mL) and morpholine (78 mg, 0.89 mmol) was added to it followed by K₃PO₄ (143 mg, 0.67 mmol). The solution was then degassed with argon and XPhos (21 mg, 0.045 mmol) was added to it. Pd₂(dba)₃ (41 mg) was then added to the reaction mixture under inert atmosphere and it was heated at 80° C. for 3 h. The reaction mixture was filtered and the organic part was washed with water (10 mL) and brine (10 mL). The solvent was evaporated under reduced pressure and the crude was further purified by column chromatography over silica gel using 40% ethyl acetate in hexane and the desired compound (Example 89) was isolated as yellow sticky gum (110 mg, 54% yield, 98.38% purity). LCMS: m/z found 453.17[M+H]⁺, rt=3.26 min (Method 2) [Waters Acquity BEH C8 column (1.7 μm, 50×2.1 mm)]; ¹H NMR (400 MHz, DMSO-d₆): δ 7.50 (d, J=4.3 Hz, 1H), 7.39 (dd, J=5.4, 8.7 Hz, 2H), 7.26 (t, J=8.8 Hz, 2H), 6.23 (d, J=4.3 Hz, 1H), 5.88-5.73 (m, 1H), 3.72 (dd, J=4.0, 5.9 Hz, 4H), 3.29-3.03 (m, 6H), 0.97 (t, J=7.0 Hz, 3H).

### (R)—N-ethyl-5-morpholino-N-(2,2,2-trifluoro-1-(4-fluorophenyl)ethyl)thiophene-2-sulfonamide (Example 90 (EN-1)) and (S)—N-ethyl-5-morpholino-N-(2,2,2-trifluoro-1-(4-fluorophenyl)ethyl)thiophene-2-sulfonamide (Example 91 (EN-2))

Chiral separation of racemic Example 89 provided both the enantiomers as mentioned below. Chiral separation method: Chiral separation was done on an Agilent 1200 series instrument. Column: CHIRALPAK IA (250×20 mm) 5μ, operating at ambient temperature and flow rate is 18.0 mL/min. Mobile phase was a mixture of 90% Hexane and 10% EtOH, held this isocratic mixture run up to 20 min at a wavelength of 310 nm.

Example 90: Light yellow sticky gum (99.12% purity). LCMS: m/z found 453.17[M+H]⁺, rt=3.26 min (Method 2) [Waters Acquity BEH C8 column (1.7 μm, 50×2.1 mm)]: ¹H NMR (400 MHz, Chloroform-d): δ 7.45-7.34 (m. 3H). 7.11-7.01 (m. 2H). 6.00 (d, J=4.3 Hz, 1H), 5.74 (q, J=8.5 Hz, 1H), 3.92-3.78 (m, 4H), 3.38-3.24 (m, 1H), 3.23-3.15 (m, 4H), 3.18-3.04 (m, 1H), 0.99 (t, J=7.1 Hz, 3H).

Example 91: Light yellow sticky gum (98.94% purity). LCMS: m/z found 453.17[M+H]⁺, rt=3.25 min (Method 2) [Waters Acquity BEH C8 column (1.7 μm, 50×2.1 mm)]; ¹H NMR (400 MHz, Chloroform-d): δ 7.45-7.34 (m, 3H), 7.05 (t, J=8.8 Hz, 2H), 6.00 (d, J=4.0 Hz, 1H), 5.74 (q, J=8.4 Hz, 1H), 3.83 (t, J=4.8 Hz, 4H), 3.39-3.24 (m, 1H), 3.22-3.04 (m, 5H), 1.00 (t, J=7.1 Hz, 3H).

### Example 92

### N-ethyl-4-morpholino-N-(2,2,2-trifluoro-1-(4-fluorophenyl)ethyl)thiophene-2-sulfonamide

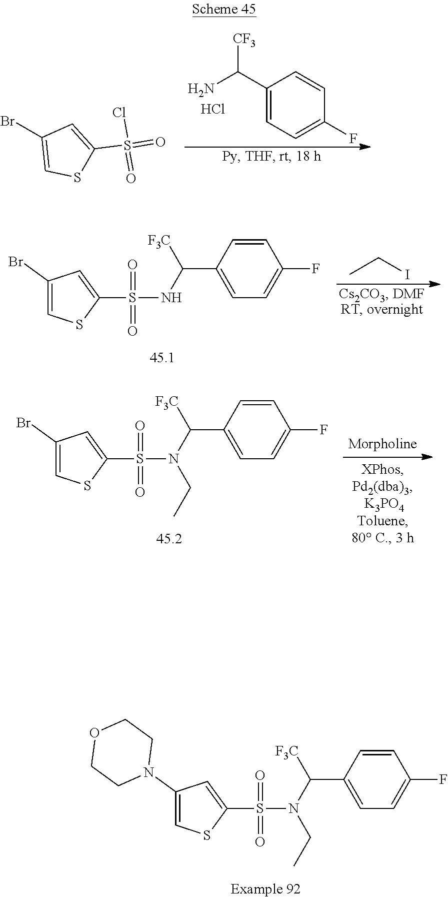

Synthesis of 4-bromo-N-(2,2,2-trifluoro-1-(4-fluorophenyl)ethyl)thiophene-2-sulfonamide 45.1: Intermediate 45.1 was synthesized from 4-bromothiophene-2-sulfonyl chloride following the protocol as described in Method O using 2,2,2-trifluoro-1-(4-fluorophenyl)ethan-1-amine hydrochloride. The compound was purified by column chromatography over silica gel using 25% ethyl acetate in hexane and isolated as yellow oil (460 mg, 57% yield). ¹H NMR (400 MHz, DMSO-d₆) δ 9.60-9.70 (m, 1H), 7.87 (s, 1H), 7.53-7.47 (m, 2H), 7.33 (s, 1H), 7.13 (t, J=8.8 Hz, 2H), 5.47-5.40 (m, 1H).

Synthesis of 4-bromo-N-ethyl-N-(2,2,2-trifluoro-1-(4-fluorophenyl)ethyl)thiophene-2-sulfonamide 45.2: The ethylation of intermediate 45.1 was performed following the protocol as described in Method D. The crude was purified by column chromatography over silica gel using 25% ethyl acetate in hexane and the desired compound intermediate 45.2 was isolated as a colorless sticky gum (280 mg, 65% yield). ¹H NMR (400 MHz, DMSO-d₆) δ 8.11 (s, 1H), 7.81 (s, 1H), 7.45-7.40 (m, 2H), 7.25 (t, J=8.5 Hz, 2H), 6.00-5.90 (m, 1H), 3.40-3.20 (m, 2H), 1.02 (t, J=6.9 Hz, 3H).

Synthesis of N-ethyl-4-morpholino-N-(2,2,2-trifluoro-1-(4-fluorophenyl)ethyl)thiophene-2-sulfonamide (Example 92): Buchwald coupling; Method W: 4-bromo-N-ethyl-N-(2,2,2-trifluoro-1-(4-fluorophenyl)ethyl)thiophene-2-sulfonamide 3.2 (200 mg, 0.45 mmol) was dissolved in toluene (1 mL) and morpholine (78 mg, 0.89 mmol) was added to it followed by K₃PO₄ (143 mg, 0.67 mmol). The solution was then degassed with argon and XPhos (21 mg, 0.045 mmol) was added to it. Pd₂(dba)₃ (41 mg) was added to the reaction mixture under inert atmosphere and it was heated at 80° C. for 3 h. The reaction mixture was filtered and the organic part was washed with water (10 mL) and brine (10 mL). The solvent was evaporated under reduced pressure and the crude was further purified by column chromatography over silica gel using 40% ethyl acetate in hexane and the desired compound Example 92 was isolated as a light yellow sticky gum (25 mg, 25% yield, 98.69% purity). LCMS: m/z found 453.19[M+H]⁺, rt=3.31 min (Method 2) [Waters Acquity BEH C8 column (1.7 μm, 50×2.1 mm)]; ¹H NMR (400 MHz, Chloroform-d) δ 7.38 (dd, J=5.1, 8.6 Hz, 2H), 7.30 (d, J=1.7 Hz, 1H), 7.06 (t, J=8.5 Hz, 2H), 6.41 (d, J=1.7 Hz, 1H), 5.74 (q, J=8.4 Hz, 1H), 3.86-3.79 (m, 4H), 3.41-3.27 (m, 1H), 3.24-3.10 (m, 1H), 3.08-3.01 (m, 4H), 1.02 (t, J=7.0 Hz, 3H).

### Example 93 and Example 94

### N-ethyl-5-morpholino-N-(2,2,2-trifluoro-1-(4-fluorophenyl)ethyl)pyridine-3-sulfonamide (Example 93) and N-ethyl-5-(pyrrolidin-1-yl)-N-(2,2,2-trifluoro-1-(4-fluorophenyl)ethyl)pyridine-3-sulfonamide (Example 94

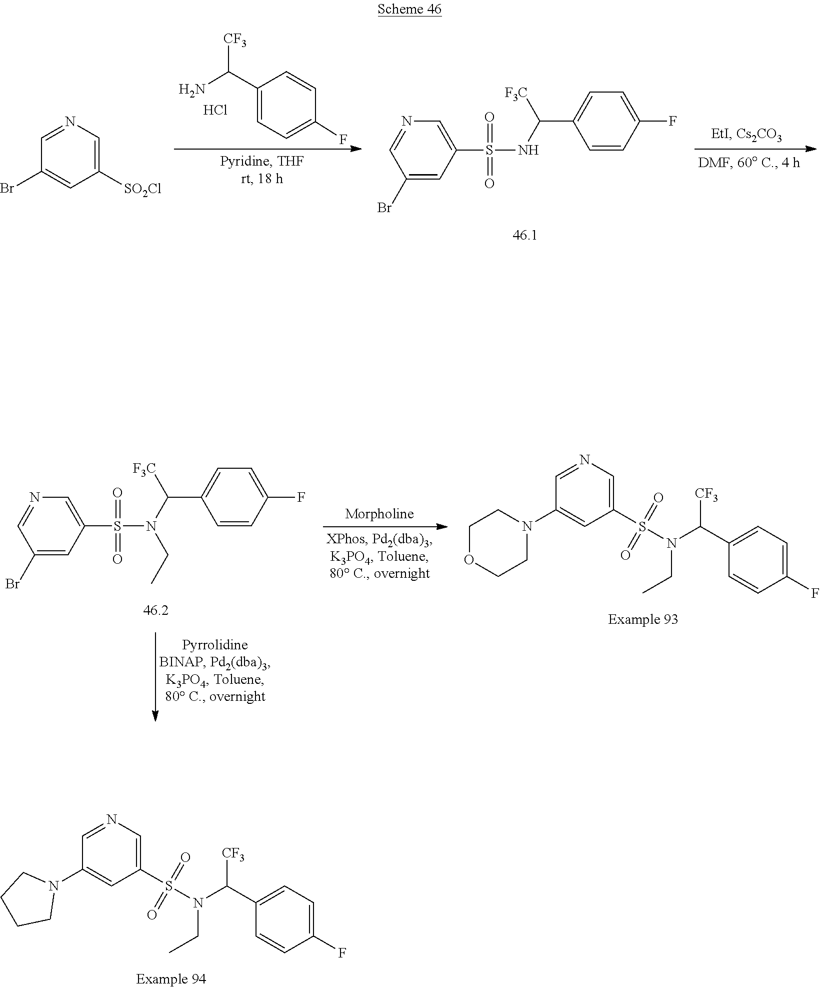

Synthesis of 5-bromo-N-(2,2,2-trifluoro-1-(4-fluorophenyl)ethyl)pyridine-3-sulfonamide 46.1: Intermediate 46.1 was synthesized from 5-bromopyridine-3-sulfonyl chloride following the protocol as described in Method O using 2,2,2-trifluoro-1-(4-fluorophenyl)ethan-1-amine hydrochloride. The compound was purified by column chromatography over silica gel using 30% ethyl acetate in hexane and isolated as yellow oil (520 mg, 64% yield). ¹H NMR (400 MHz, DMSO-d6): δ 9.70-9.60 (m, 1H), 8.82 (d, J=2.1 Hz, 1H), 8.75 (d, J=1.9 Hz, 1H), 8.08 (t, J=2.1 Hz, 1H), 7.47-7.42 (m, 2H), 7.10 (t, J=8.8 Hz, 2H), 5.60-5.40 (m, 1H).

Synthesis of 5-bromo-N-ethyl-N-(2,2,2-trifluoro-1-(4-fluorophenyl)ethyl)pyridine-3-sulfonamide 46.2: The ethylation of intermediate 46.1 was performed following the protocol as described in Method D at 60° C. The crude residue was purified by column chromatography over silica gel using 30% ethyl acetate in hexane and the desired compound intermediate 46.2 was isolated as yellow sticky gum (180 mg, 32% yield). ¹H NMR (400 MHz, DMSO-d6): δ 9.03 (s, 1H), 8.96 (s, 1H), 8.38 (s, 1H), 7.47-7.40 (m, 2H), 7.26 (t, J=8.5 Hz, 2H), 3.60-3.45 (m, 2H), 1.12 (t, J=7.0 Hz, 3H).

Synthesis of N-ethyl-5-morpholino-N-(2,2,2-trifluoro-1-(4-fluorophenyl)ethyl)pyridine-3-sulfonamide (Example 93): compound Example 93 was synthesized from intermediate 4.2 following a method analogous to that described in Method W (Buchwald coupling) using morpholine. The crude was purified by column chromatography over silica gel using 40% ethyl acetate in hexane and isolated as light yellow sticky gum (40 mg, 40% yield, 97.39% purity). LCMS: m/z found 448.25 [M+H]⁺, rt=3.04 min (Method 2) [Waters Acquity BEH C8 column (1.7 μm, 50×2.1 mm)]; ¹H NMR (400 MHz, Chloroform-d): δ 8.53 (d, J=1.6 Hz, 1H), 8.43 (t, J=2.7 Hz, 1H), 7.51-7.42 (m, 3H), 7.15-7.04 (m, 2H), 5.83 (q, J=8.3 Hz, 1H), 3.92-3.84 (m, 4H), 3.42-3.28 (m, 1H), 3.30-3.20 (m, 4H), 3.23-3.09 (m, 1H), 0.93 (t, J=7.1 Hz, 3H).

Synthesis of N-ethyl-5-(pyrrolidin-1-yl)-N-(2,2,2-trifluoro-1-(4-fluorophenyl)ethyl)pyridine-3-sulfonamide (Example 94): Example 94 was synthesized from intermediate 46.2 following a method analogous to that described in Method W (Buchwald coupling) using pyrrolidine and BINAP as ligand. The crude was purified by column chromatography over silica gel using 40% ethyl acetate in hexane and isolated as a colourless sticky gum (70 mg, 36% yield, 99.56% purity). LCMS: m/z found 432.2 [M+H]⁺, rt=3.07 min (Method 9) [Waters Xbridge C18 column (3.5 μm, 50×3 mm)]; ¹H NMR (400 MHz, DMSO-d₆): δ 8.24-8.21 (m, 1H), 8.13 (d, J=2.4 Hz, 1H), 7.46 (dd, J=5.3, 8.6 Hz, 2H), 7.23 (t, J=8.7 Hz, 2H), 7.14-7.08 (m, 1H), 6.06 (q, J=8.8 Hz, 1H), 3.42-3.31 (m, 1H), 3.32-3.14 (m, 5H), 2.02-1.93 (m, 4H), 0.96 (t, J=6.9 Hz, 3H).

### Example 95 and Example 96

### (S)—N-ethyl-5-morpholino-N-(2,2,2-trifluoro-1-(4-fluorophenyl)ethyl)pyridine-3-sulfonamide (Example 95 (EN-1)) and (R)—N-ethyl-5-morpholino-N-(2,2,2-trifluoro-1-(4-fluorophenyl)ethyl)pyridine-3-sulfonamide (Example 96 (EN-2

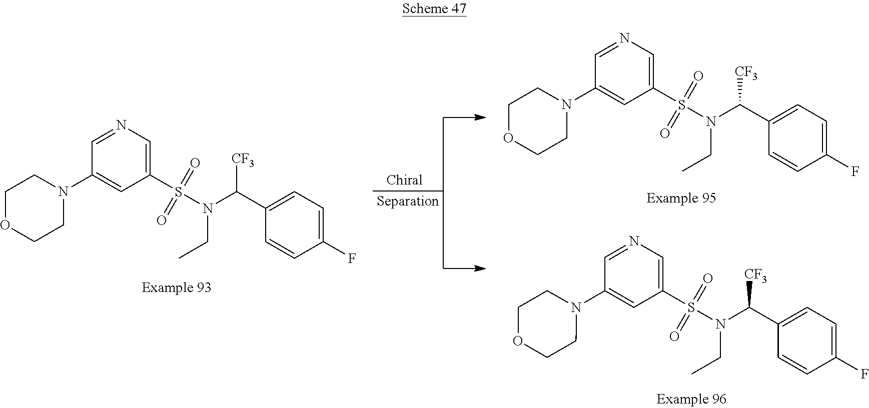

Chiral separation of racemic Example 93 provided both the enantiomers as mentioned below. Chiral separation method: Chiral separation was done on an Agilent 1200 series instrument. Column: CHIRALPAK IG (250×21 mm) 5μ, operating at ambient temperature and flow rate is 21.0 mL/min. Mobile phase was a mixture of 60% Hexane and 40% EtOH, this isocratic mixture held to run up to 25 min at a wavelength of 264 nm.

Example 95: Light yellow sticky gum (96.52% purity). LCMS: m/z found 448.2 [M+H]⁺, rt=3.05 min (Method 2) [Waters Acquity BEH C8 column (1.7 μm, 50×2.1 mm)]; ¹H NMR (400 MHz, DMSO-d₆): δ 8.53 (d, J=2.7 Hz, 1H), 8.42 (d, J=1.5 Hz, 1H), 7.61 (t, J=2.4 Hz, 1H), 7.45 (dd, J=5.3, 8.6 Hz, 2H), 7.23 (t, J=8.8 Hz, 2H), 6.09 (q, J=8.8 Hz, 1H), 3.75 (t, J=5.0 Hz, 4H), 3.44-3.33 (m, 1H), 3.29-3.15 (m, 5H), 0.97 (t, J=7.0 Hz, 3H).

Example 96: Yellow sticky gum (98.03% purity). LCMS: m/z found 448.2 [M+H]⁺, rt=3.05 min (Method 2) [Waters Acquity BEH C8 column (1.7 μm, 50×2.1 mm)]; ¹H NMR (400 MHz, DMSO-d₆): δ 8.53 (d, J=2.6 Hz, 1H), 8.42 (d, J=1.5 Hz, 1H), 7.61 (t, J=2.4 Hz, 1H), 7.45 (dd, J=5.3, 8.6 Hz, 2H), 7.23 (t, J=8.8 Hz, 2H), 6.09 (q, J=8.6 Hz, 1H), 3.75 (t, J=5.0 Hz, 4H), 3.53-3.31 (m, 1H), 3.29-3.17 (m, 5H), 0.97 (t, J=7.0 Hz, 3H).

### Example 97 and Example 98

### (S)—N-ethyl-5-(pyrrolidin-1-yl)-N-(2,2,2-trifluoro-1-(4-fluorophenyl)ethyl)pyridine-3-sulfonamide (Example 97 (EN-1)) and (R)—N-ethyl-5-(pyrrolidin-1-yl)-N-(2,2,2-trifluoro-1-(4-fluorophenyl)ethyl)pyridine-3-sulfonamide (Example 98 (EN-2

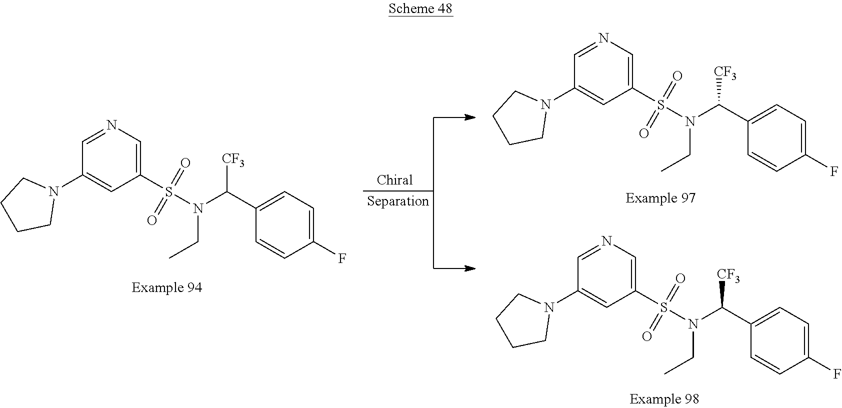

Chiral separation of racemic Example 94 provided both the enantiomers as mentioned below. Chiral separation method: Chiral separation was done on an Agilent 1200 series instrument. Column: CHIRALPAK IG (250×21 mm) 5μ, operating at ambient temperature and flow rate is 21.0 mL/min. Mobile phase was a mixture of 60% Hexane and 40% EtOH, this isocratic mixture held to run up to 25 min at a wavelength of 272 nm.

Example 97 (EN-1): Colorless sticky gum (99.11% purity). LCMS: m/z found 432.3 [M+H]⁺, rt=3.46 min (Method 2) [Waters Acquity BEH C18 column (1.7 μm, 50×2.1 mm)]; ¹H NMR (400 MHz, DMSO-d₆): δ 8.23 (d, J=2.0 Hz, 1H), 8.13 (d, J=2.6 Hz, 1H), 7.47 (dd, J=5.3, 8.6 Hz, 2H), 7.23 (t, J=8.8 Hz, 2H), 7.11 (t, J=2.4 Hz, 1H), 6.06 (q, J=8.9 Hz, 1H), 3.41-3.32 (m, 2H), 3.31-3.15 (m, 4H), 2.02-1.94 (m, 4H), 0.96 (t, J=6.8 Hz, 3H).

Example 98 (EN-2): Colorless sticky gum (98.82% purity). LCMS: m/z found 432.3 [M+H]⁺, rt=3.46 min (Method 2) [Waters Acquity BEH C18 column (1.7 μm, 50×2.1 mm)]; ¹H NMR (400 MHz, Chloroform-d): δ 8.33 (s, 1H), 8.10 (d, J=2.6 Hz, 1H), 7.44 (t, J=5.1 Hz, 2H), 7.18-7.01 (m, 3H), 5.83 (q, J=8.4 Hz, 1H), 3.43-3.25 (m, 5H), 3.20-3.09 (m, 1H), 2.11-2.03 (m, 4H), 0.94 (t, J=7.0 Hz, 3H).

### Example 99

### N-ethyl-5-(oxetan-3-yl)-N-(2,2,2-trifluoro-1-(4-fluorophenyl)ethyl)pyridine-3-sulfonamide

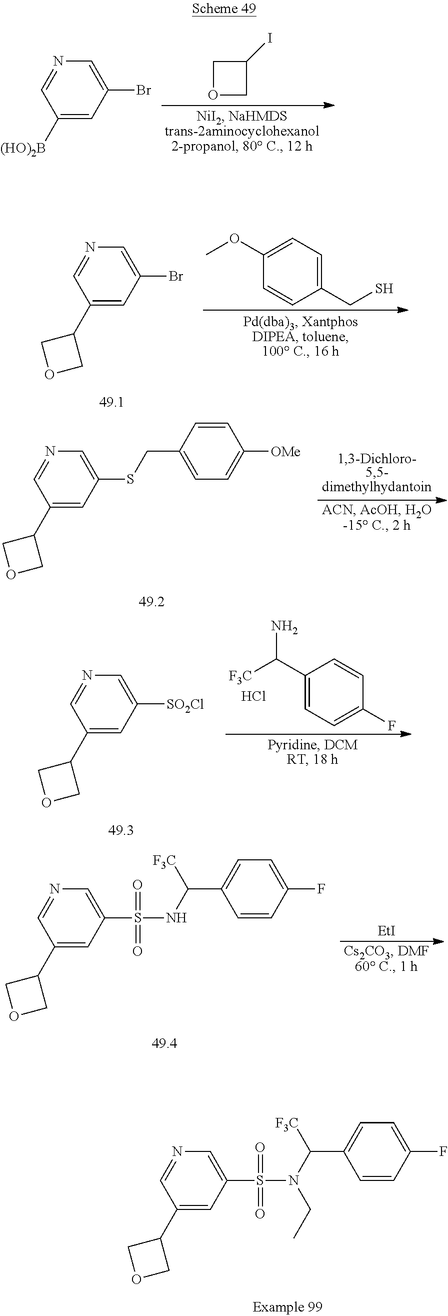

Synthesis of 3-bromo-5-(oxetan-3-yl)pyridine 49.1: Suzuki coupling; Method V: (5-bromopyridin-3-yl)boronic acid (2 g, 9.91 mmol) was dissolved in 2-propanol (5 mL) and trans-2aminocyclohexanol (45 mg, 0.3 mmol) was added to it. The solution was degassed with argon and NiI₂ (93 mg, 0.3 mmol) was added to it, followed by NaHMDS (5 mL). The reaction mixture was stirred at RT for 10 mins and 3-iodooxetane (1.27 g, 6.94 mmol) in 2-Propanol (10 mL) was added to it. The reaction mixture was then heated at 80° C. overnight. After filtration, the filtrate was evaporated under reduced pressure. The crude residue was purified by column chromatography over silica gel using 40% ethyl acetate in hexane and isolated as a white sticky solid (1.0 g, 47% yield yield). ¹H NMR (400 MHz, DMSO-d6): δ 8.62-8.60 (m, 1H), 8.59-8.56 (m, 1H), 8.18-8.15 (m, 1H), 5.0-4.90 (m, 2H), 4.65 (t, J=6.4 Hz, 2H), 4.40-4.25 (m, 1H).

Synthesis of 3-((4-methoxybenzyl)thio)-5-(oxetan-3-yl)pyridine 49.2: Intermediate 49.2 was synthesized from intermediate 49.1 following a method analogous to that described in Method I using (4-methoxyphenyl)methanethiol. The crude residue was purified by column chromatography over silica gel using 20% ethyl acetate in hexane and isolated as a yellow sticky gum (150 mg, 45% yield). ¹H NMR (400 MHz, DMSO-d6): δ 8.40-8.30 (m, 2H), 7.80 (d, J=1.9 Hz, 1H), 7.25 (d, J=8.5 Hz, 2H), 6.85 (d, J=8.6 Hz, 2H), 5.0-4.80 (m, 2H), 4.58 (t, J=6.6 Hz, 2H), 4.30-4.20 (brs, 2H), 3.71 (s, 3H).

Synthesis of 5-(oxetan-3-yl)pyridine-3-sulfonyl chloride 49.3: Sulfonyl chloride 49.3 was synthesized from intermediate 49.2 following the procedure described in Method T. The crude sulfonyl chloride was used immediately in the following step without further purification.

Synthesis of 5-(oxetan-3-yl)-N-(2,2,2-trifluoro-1-(4-fluorophenyl)ethyl)pyridine-3-sulfonamide 49.4: Intermediate 49.4 was synthesized from sulfonyl chloride 49.3 following the protocol as described in Method O using 2,2,2-trifluoro-1-(4-fluorophenyl)ethan-1-amine hydrochloride. The compound was purified by column chromatography over silica gel using 35% ethyl acetate in hexane and isolated as brown gum (120 mg, 36% yield). ¹H NMR (400 MHz, DMSO-d6): δ 9.53-9.50 (m, 1H), 8.70-8.60 (m, 2H), 8.05 (brs, 1H), 7.45-7.40 (m, 2H), 7.1-7.0 (m, 2H) 5.50-5.40 (m, 1H), 4.91 (t, J=7.9 Hz, 2H), 4.50-4.40 (m, 2H), 4.30-4.20 (m, 1H).

Synthesis of N-ethyl-5-(oxetan-3-yl)-N-(2,2,2-trifluoro-1-(4-fluorophenyl)ethyl)pyridine-3-sulfonamide (Example 99): The ethylation of intermediate 49.4 was performed following the protocol as described in Method D at 60° C. The crude residue was purified by Reverse Phase Prep-HPLC and the desired compound was isolated as a colourless sticky gum (30 mg, 20% yield, 98.37% purity). Preparative HPLC was done on Waters auto purification instrument. Column: XBRIDGE PREP C18 OBD (250×19 mm, 5μ), operating at ambient temperature and flow rate of 16.0 ml/min. Mobile phase: A=20 mM NH₄HCO₃ in water, B=METHANOL; Gradient Profile: Mobile phase initial composition of 80% A and 20% B, then to 40% A and 60% B in 4 min., then to 24% A and 76% B in 20 min, then to 5% A and 95% B in 21 min, held this composition up to 24 min for column washing, then returned to initial composition in 25 min and held for 28 min). LCMS: m/z found 419.2 [M+H]⁺, rt=2.74 min (Method 9) [Waters Xbridge C18 column (3.5 μm, 50×3 mm)]; ¹H NMR (400 MHz, Chloroform-d): δ 9.01 (s, 1H), 8.77 (s, 1H), 8.26 (s, 1H), 7.52-7.46 (m, 2H), 7.11 (t, J=8.6 Hz, 2H), 5.89-5.80 (m, 1H), 5.16 (t, J=7.6 Hz, 2H), 4.70 (q, J=6.0 Hz, 2H), 4.29 (t, J=7.1 Hz, 1H), 3.47-3.11 (m, 2H), 0.93 (t, J=7.0 Hz, 3H).

### Example 100 to Example 102

### N-ethyl-1-methyl-6-oxo-N-(2,2,2-trifluoro-1-(4-fluorophenyl)ethyl)-1,6-dihydropyridine-3-sulfonamide (Example 100), (R)—N-ethyl-1-methyl-6-oxo-N-(2,2,2-trifluoro-1-(4-fluorophenyl)ethyl)-1,6-dihydropyridine-3-sulfonamide (Example 101) and (S)—N-ethyl-1-methyl-6-oxo-N-(2,2,2-trifluoro-1-(4-fluorophenyl)ethyl)-1,6-dihydropyridine-3-sulfonamide (Example 102)

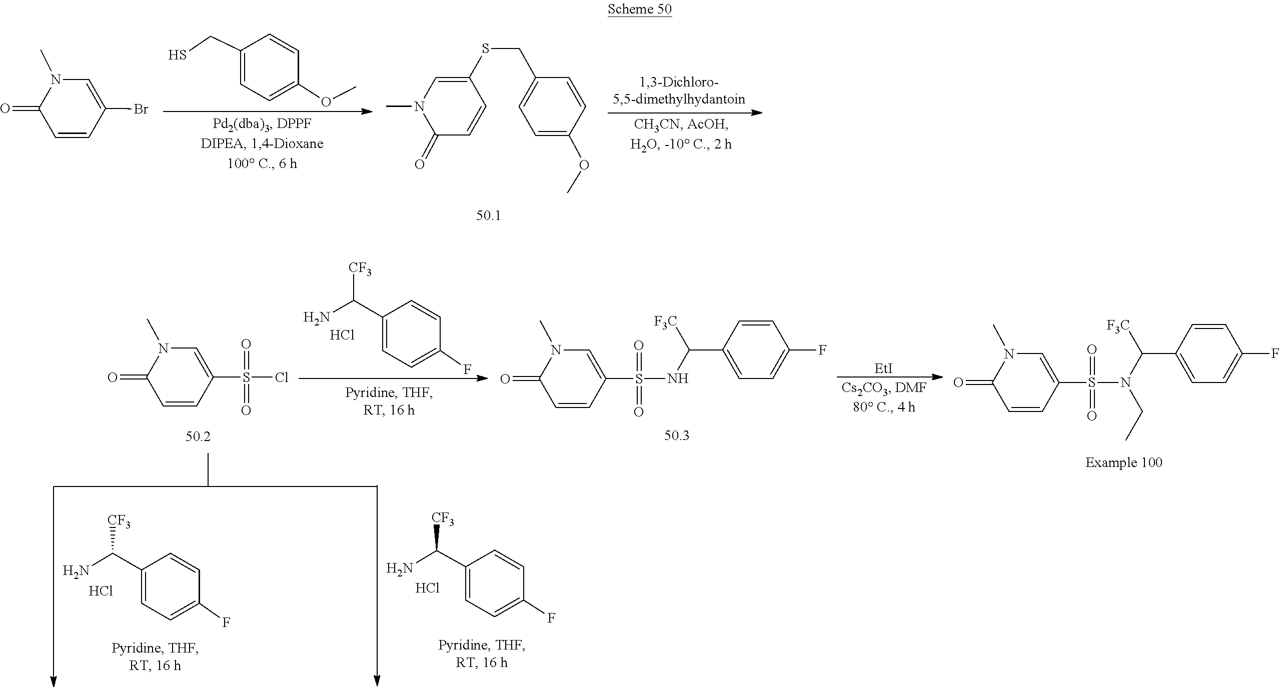

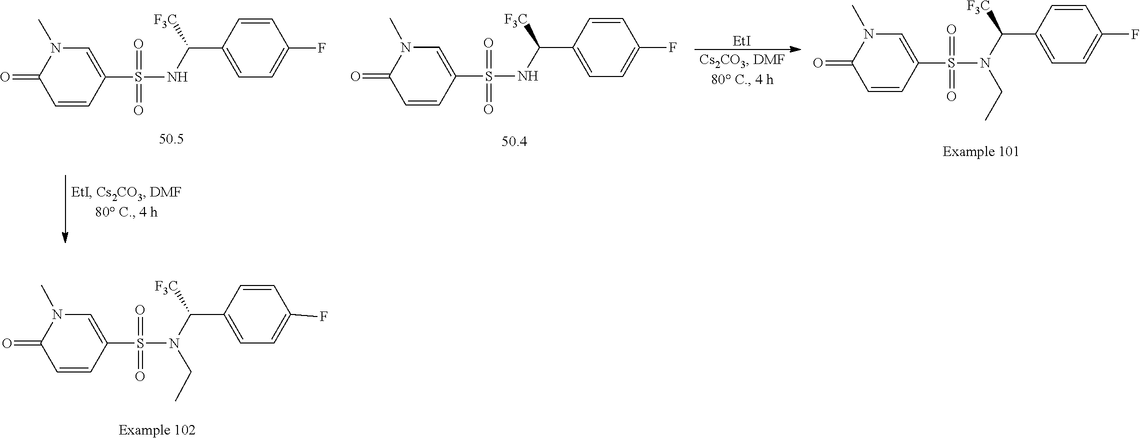

Synthesis of 5-((4-methoxybenzyl)thio)-1-methylpyridin-2(1H)-one 50.1: Intermediate 50.1 was synthesized from 5-bromo-1-methylpyridin-2(1H)-one following a method analogous to that described in Method I using (4-methoxyphenyl)methanethiol. The crude residue was purified by column chromatography over silica gel using 20% ethyl acetate in hexane and isolated as a yellow sticky gum (600 mg, 86% yield). ¹H NMR (400 MHz, DMSO-d6): δ 7.70-7.65 (brs, 1H), 7.35-7.30 (m, 1H), 7.10 (d, J=8.3 Hz, 2H), 6.84 (d, J=8.3 Hz, 2H), 6.31 (d, J=9.5 Hz, 1H), 3.90 (s, 2H), 3.71 (s, 3H), 3.31 (s, 3H).

Synthesis of 1-methyl-6-oxo-1,6-dihydropyridine-3-sulfonyl chloride 50.2: Sulfonyl chloride 50.2 was synthesized from intermediate 50.1 following the procedure described in Method T. The crude sulfonyl chloride was used immediately in the following step without further purification.

Synthesis of 1-methyl-6-oxo-N-(2,2,2-trifluoro-1-(4-fluorophenyl)ethyl)-1,6-dihydropyridine-3-sulfonamide 50.3: Intermediate 50.3 was synthesized from sulfonyl chloride 50.2 following the protocol as described in Method O using 2,2,2-trifluoro-1-(4-fluorophenyl)ethan-1-amine hydrochloride. The compound was purified by column chromatography over silica gel using 35% ethyl acetate in hexane and isolated as a brown gum (150 mg, 57% yield). ¹H NMR (400 MHz, DMSO-d6): δ 9.25-9.20 (m, 1H), 8.14 (d, J=2.6 Hz, 1H), 7.51-7.45 (m, 2H), 7.32-7.10 (m, 1H), 7.14 (t, J=8.7 Hz, 3H), 6.23 (d, J=9.6 Hz, 1H), 5.28-5.16 (m, 1H), 3.36 (s, 3H).

Synthesis of N-ethyl-1-methyl-6-oxo-N-(2,2,2-trifluoro-1-(4-fluorophenyl)ethyl)-1,6-dihydropyridine-3-sulfonamide (Example 100): The ethylation of intermediate 50.3 was performed following the protocol as described in Method D at 60° C. The crude residue was purified by column chromatography over silica gel using 35% ethyl acetate in hexane and isolated as a light-yellow gum (50 mg, 31% yield, 99.61% purity). LCMS: m/z found 393.1 [M+H]⁺, rt=2.70 min (Method 9) [Waters Xbridge C18 column (3.5 μm, 50×3 mm)]; ¹H NMR (400 MHz, Chloroform-d) δ 8.00 (d, J=2.6 Hz, 1H), 7.56 (dd, J=2.7, 9.6 Hz, 1H), 7.53-7.45 (m, 2H), 7.11 (t, J=8.6 Hz, 2H), 6.59 (d, J=9.7 Hz, 1H), 5.74 (q, J=8.2 Hz, 1H), 3.59 (s, 3H), 3.40-3.26 (m, 1H), 3.19-3.05 (m, 1H), 0.93 (t, J=7.1 Hz, 3H).

Synthesis of (R)-1-methyl-6-oxo-N-(2,2,2-trifluoro-1-(4-fluorophenyl)ethyl)-1,6-dihydropyridine-3-sulfonamide 50.4: Intermediate 50.4 was synthesized from sulfonyl chloride 50.2 following the protocol as described in Method O using (R)-2,2,2-trifluoro-1-(4-fluorophenyl)ethan-1-amine hydrochloride. The compound was purified by column chromatography over silica gel using 35% ethyl acetate in hexane and isolated as yellow gum (130 mg, 53% yield). ¹H NMR (400 MHz, DMSO-d6): δ 9.25-9.20 (m, 1H), 8.14 (d, J=2.6 Hz, 1H), 7.51-7.45 (m, 2H), 7.32-7.10 (m, 1H), 7.14 (t, J=8.7 Hz, 3H), 6.23 (d, J=9.6 Hz, 1H), 5.28-5.16 (m, 1H), 3.36 (s, 3H).

Synthesis of (R)—N-ethyl-1-methyl-6-oxo-N-(2,2,2-trifluoro-1-(4-fluorophenyl)ethyl)-1,6-dihydropyridine-3-sulfonamide (Example 101): The ethylation of intermediate 50.4 was performed following the protocol as described in Method D at 60° C. The crude residue was purified by column chromatography over silica gel using 35% ethyl acetate in hexane and further purified by Prep-HPLC Chiral SFC. Compound was isolated as light yellow gum (99.73% purity). LCMS: m/z found 393.16 [M+H]⁺, rt=2.43 min (Method 2) [Waters Acquity BEH C8 column (1.7 μm, 50×2.1 mm)]; ¹H NMR (400 MHz, DMSO-d₆): δ 8.49 (d, J=2.8 Hz, 1H), 7.66 (dd, J=2.9, 9.9 Hz, 1H), 7.51 (dd, J=5.3, 8.3 Hz, 2H), 7.28 (t, J=8.6 Hz, 2H), 6.45 (d, J=9.6 Hz, 1H), 5.88 (q, J=8.6 Hz, 1H), 3.49 (s, 3H), 3.42-3.32 (m, 1H), 3.26-3.16 (m, 1H), 0.98 (t, J=7.2 Hz, 3H).

Chiral separation method: Chiral separation of was done on Thar SFC-80 series instrument by using CHIRALPAK IG column (21 mm×250 mm), 5μ, operating at 35° C. temperature, maintaining flow rate of 50 gm/min using 80% C02 in super critical state & 20% of (0.3% Ipamine in Methanol) as mobile phase, Run this isocratic mixture up to 8 min and also maintained the isobaric condition of 100 bar at 248 nm wavelength.

Synthesis of (S)-1-methyl-6-oxo-N-(2,2,2-trifluoro-1-(4-fluorophenyl)ethyl)-1,6-dihydropyridine-3-sulfonamide 50.5: Intermediate 50.5 was synthesized from sulfonyl chloride 50.2 following the protocol as described in Method O using (S)-2,2,2-trifluoro-1-(4-fluorophenyl)ethan-1-amine hydrochloride. The compound was purified by column chromatography over silica gel using 35% ethyl acetate in hexane and isolated as a brown gum (120 mg, 50% yield). ¹H NMR (400 MHz, DMSO-d6): δ 9.25-9.20 (m, 1H), 8.14 (d, J=2.6 Hz, 1H), 7.51-7.45 (m, 2H), 7.32-7.10 (m, 1H), 7.14 (t, J=8.7 Hz, 3H), 6.23 (d, J=9.6 Hz, 1H), 5.28-5.16 (m, 1H), 3.36 (s, 3H).

Synthesis of (S)—N-ethyl-1-methyl-6-oxo-N-(2,2,2-trifluoro-1-(4-fluorophenyl)ethyl)-1,6-dihydropyridine-3-sulfonamide (Example 102): The ethylation of intermediate 50.5 was performed following the protocol as described in Method D at 60° C. The crude was purified by column chromatography over silica gel using 35% ethyl acetate in hexane and further purified by Prep-HPLC Chiral SFC. Compound was isolated as light-yellow sticky gum (99.88% purity). LCMS: m/z found 393.18 [M+H]⁺, rt=2.43 min (Method 2) [Waters Acquity BEH C8 column (1.7 μm, 50×2.1 mm)]; ¹H NMR (400 MHz, DMSO-d₆): δ 8.49 (d, J=2.8 Hz, 1H), 7.66 (dd, J=2.9, 9.9 Hz, 1H), 7.51 (dd, J=5.3, 8.3 Hz, 2H), 7.28 (t, J=8.6 Hz, 2H), 6.45 (d, J=9.6 Hz, 1H), 5.97-5.78 (m, 1H), 3.49 (s, 3H), 3.42-3.32 (m, 1H), 3.26-3.16 (m, 1H), 0.97 (t, J=7.2 Hz, 3H).

Chiral separation method: Chiral separation was done on Thar SFC-80 series instrument by using CHIRALPAK IG column (21 mm×250 mm), 5μ, operating at 35° C. temperature, maintaining flow rate of 50 gm/min, using 80% C02 in super critical state & 20% of (0.3% Ipamine in Methanol) as mobile phase, Run this isocratic mixture up to 9 min and also maintained the isobaric condition of 100 bar at 248 nm wavelength.

### Example 103

### N-ethyl-1-methyl-2-oxo-N-(2,2,2-trifluoro-1-(4-fluorophenyl)ethyl)-1,2-dihydropyridine-4-sulfonamide (Example 103)

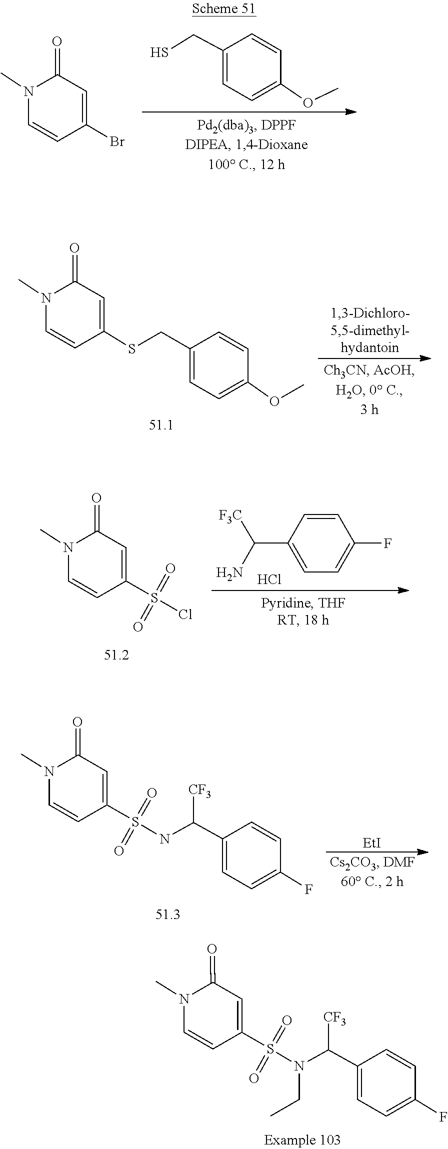

Synthesis of 4-((4-methoxybenzyl)thio)-1-methylpyridin-2(1H)-one 51.1: Intermediate 51.1 was synthesized from 4-bromo-1-methylpyridin-2(1H)-one following a method analogous to that described in Method I using (4-methoxyphenyl)methanethiol. The crude was isolated as brown sticky solid (300 mg, 86% yield) and used in the following step without further purification.

Synthesis of 1-methyl-2-oxo-1,2-dihydropyridine-4-sulfonyl chloride 50.2: Sulfonyl chloride 50.2 was synthesized from intermediate 50.1 following the procedure described in Method T. The crude sulfonyl chloride was used immediately in the following step without further purification.

Synthesis of 1-methyl-2-oxo-N-(2,2,2-trifluoro-1-(4-fluorophenyl)ethyl)-1,2-dihydropyridine-4-sulfonamide 50.3: Intermediate 50.3 was synthesized from sulfonyl chloride 50.2 following the protocol as described in Method O using 2,2,2-trifluoro-1-(4-fluorophenyl)ethan-1-amine hydrochloride. The crude residue was used in the following step without further purification.

Synthesis of N-ethyl-1-methyl-2-oxo-N-(2,2,2-trifluoro-1-(4-fluorophenyl)ethyl)-1,2-dihydropyridine-4-sulfonamide (Example 103): The ethylation of intermediate 50.3 was performed following the protocol as described in Method D at 60° C. The crude was purified by Reverse phase Prep-HPLC and the desired compound was isolated as a white Solid (10 mg, 40% yield, 98.69% purity). LCMS: m/z found 393.1 [M+H]⁺, rt=2.62 min (Method 9) [Waters Xbridge C18 column (3.5 μm, 50×3 mm)]; ¹H NMR (400 MHz, DMSO-d₆): δ 7.93 (d, J=7.1 Hz, 1H), 7.53 (dd, J=5.2, 8.7 Hz, 2H), 7.28 (t, J=8.8 Hz, 2H), 6.86 (d, J=2.2 Hz, 1H), 6.57 (dd, J=2.3, 7.1 Hz, 1H), 6.01 (q, J=8.6 Hz, 1H), 3.46 (s, 3H), 3.42-3.31 (m, 1H), 3.31-3.22 (m, 1H), 0.96 (t, J=7.0 Hz, 3H).

### Example 104 to Example 106

### 5-Cyano-N-ethyl-N-(2,2,2-trifluoro-1-(p-tolyl)ethyl)pyridine-3-sulfonamide (Example 104), (S)-5-Cyano-N-ethyl-N-(2,2,2-trifluoro-1-(p-tolyl)ethyl)pyridine-3-sulfonamide (Example 105 (EN-1)) and (R)-5-Cyano-N-ethyl-N-(2,2,2-trifluoro-1-(p-tolyl)ethyl)pyridine-3-sulfonamide (Example 106 (EN-2

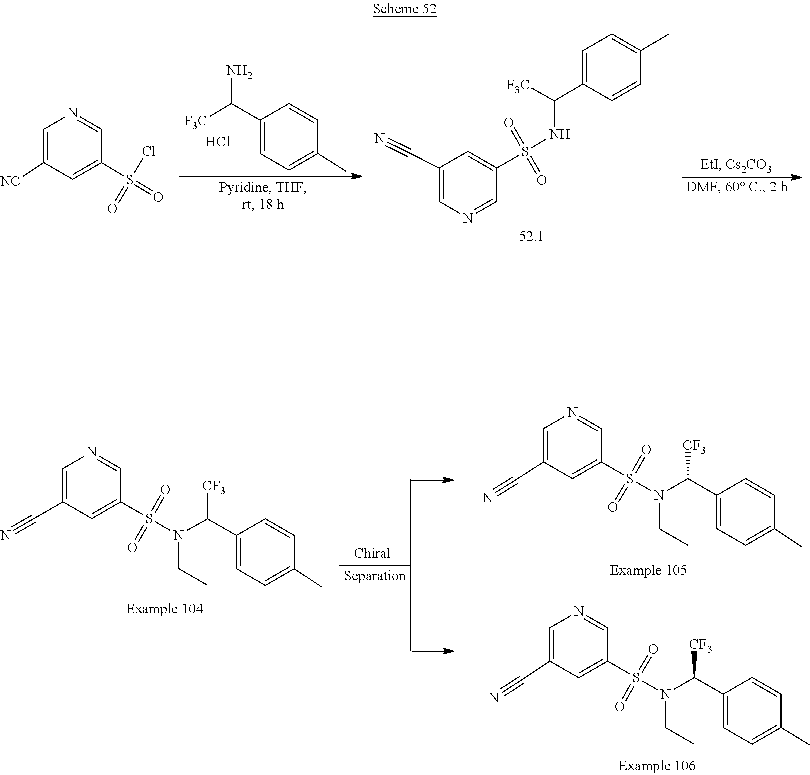

Synthesis of 5-cyano-N-(2,2,2-trifluoro-1-(p-tolyl)ethyl)pyridine-3-sulfonamide: Intermediate 52.1 was synthesized from 5-cyanopyridine-3-sulfonyl chloride following the protocol as described in Method O using 2,2,2-trifluoro-1-(p-tolyl)ethan-1-amine hydrochloride. The compound was purified by column chromatography over silica gel using 25% ethyl acetate in hexane and isolated as a colourless oil (150 mg, 48% yield). ¹H NMR (400 MHz, DMSO-d₆): δ 9.70-9.65 (brs, 1H), 9.10 (s, 1H), 8.99 (s, 1H), 8.32-8.25 (m, 1H), 7.22 (d, J=7.7 Hz, 2H), 7.03 (d, J=7.0 Hz, 2H), 5.40-5.25 (m, 1H), 2.23 (s, 3H).

Synthesis of 5-cyano-N-ethyl-N-(2,2,2-trifluoro-1-(p-tolyl)ethyl)pyridine-3-sulfonamide (Example 104): The ethylation of intermediate 52.1 was preformed following the protocol as described in Method D at 60° C. using Cs2CO3. The crude was purified by column chromatography over silica gel using 20% ethyl acetate in hexane and the desired compound Example 104 was isolated as a light yellow sticky gum (50 mg, 56% yield, 99.51% purity). LCMS: m/z found 384.0 [M+H]⁺, rt=3.65 min (Method 4) [Waters Xbridge C18 column (5 μm, 50×4.6 mm)]; ¹H NMR (400 MHz, DMSO-d₆) δ 9.32 (d, J=2.1 Hz, 1H), 9.29 (d, J=1.6 Hz, 1H), 8.83 (t, J=2.1 Hz, 1H), 7.25 (d, J=8.0 Hz, 2H), 7.19 (d, J=8.1 Hz, 2H), 5.96 (q, J=8.8 Hz, 1H), 3.48-3.26 (m, 2H), 2.29 (s, 3H), 1.00 (t, J=7.0 Hz, 3H).

Chiral separation of racemic Example 104 provided both the enantiomers as mentioned below. Chiral separation method: Chiral separation was done on Thar SFC-80 series instrument by using CHIRALPAK IG column (21 mm×250 mm), 5μ, operating at 35° C. temperature, maintaining a flow rate of 50 gm/min, using 80% CO₂ in super critical state and 20% of Methanol as a mobile phase. This isocratic mixture was run up to 9 min maintaining the isobaric condition of 100 bar at 230 nm wavelength.

5-Cyano-N-ethyl-N-(2,2,2-trifluoro-1-(p-tolyl)ethyl)pyridine-3-sulfonamide (Example 105 (EN-1)): Light yellow sticky gum (98.18% purity). LCMS: m/z found 384.3 [M+H]⁺, rt=3.49 min (Method 2) [Waters Acquity BEH C18 column (1.7 μm, 50×2.1 mm)]; ¹H NMR (400 MHz, Chloroform-d) δ 9.21 (d, J=2.2 Hz, 1H), 9.02 (d, J=1.9 Hz, 1H), 8.29 (t, J=2.0 Hz, 1H), 7.33 (d, J=8.0 Hz, 2H), 7.21 (d, J=8.1 Hz, 2H), 5.73 (q, J=8.4 Hz, 1H), 3.46-3.20 (m, 2H), 2.37 (s, 3H), 0.96 (t, J=7.0 Hz, 3H)

5-Cyano-N-ethyl-N-(2,2,2-trifluoro-1-(p-tolyl)ethyl)pyridine-3-sulfonamide (Example 106 (EN-2)): Colourless sticky gum (99.89% purity). LCMS: m/z found 384.3 [M+H]⁺, rt=3.49 min (Method 2) [Waters Acquity BEH C18 column (1.7 μm, 50×2.1 mm)]; ¹H NMR (400 MHz, Chloroform-d): δ 9.21 (d, J=2.1 Hz, 1H), 9.01 (d, J=1.9 Hz, 1H), 8.29 (t, J=2.0 Hz, 1H), 7.33 (d, J=8.0 Hz, 2H), 7.29-7.18 (m, 2H), 5.73 (q, J=8.4 Hz, 1H), 3.48-3.18 (m, 2H), 2.37 (s, 3H), 0.96 (t, J=7.0 Hz, 3H).

### Example 107 to Example 109

### 5-Cyano-N-ethyl-N-(2,2,2-trifluoro-1-(2-methoxyphenyl)ethyl)pyridine-3-sulfonamide (Example 107), (S)-5-cyano-N-ethyl-N-(2,2,2-trifluoro-1-(2-methoxyphenyl)ethyl)pyridine-3-sulfonamide (Example 108 (EN-1)) and (R)-5-cyano-N-ethyl-N-(2,2,2-trifluoro-1-(2-methoxyphenyl)ethyl)pyridine-3-sulfonamide (Example 109 (EN-2

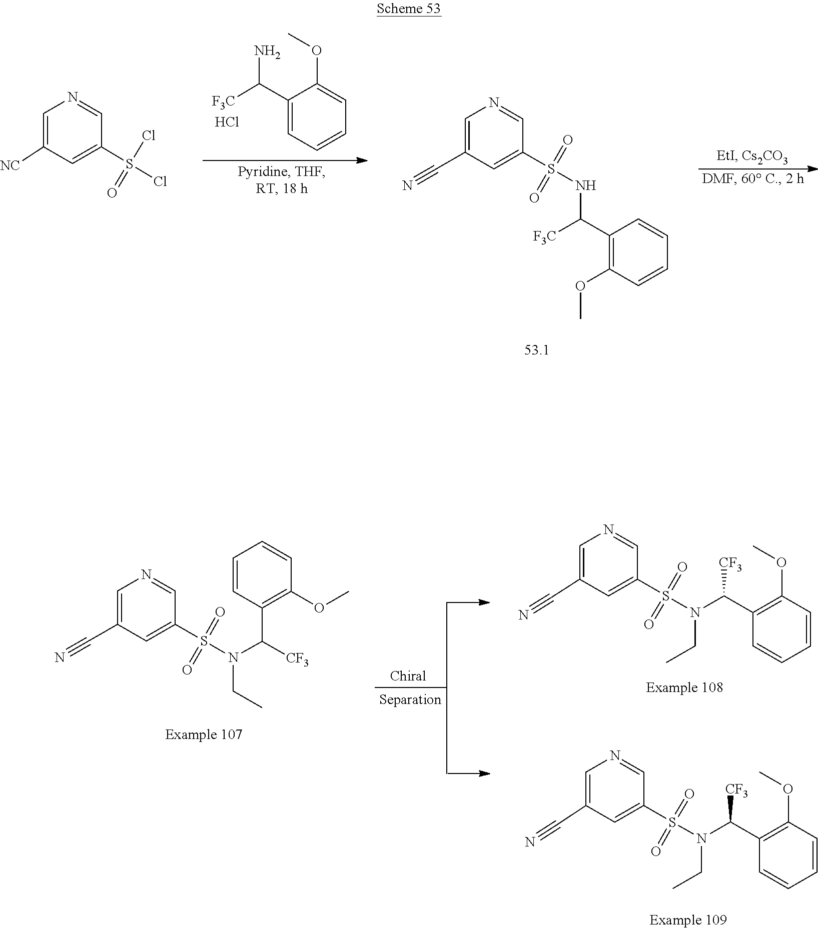

Synthesis of 5-cyano-N-(2,2,2-trifluoro-1-(2-methoxyphenyl)ethyl)pyridine-3-sulfonamide: Intermediate 11.1 was synthesized from 5-cyanopyridine-3-sulfonyl chloride following the protocol as described in Method O using 2,2,2-trifluoro-1-(2-methoxyphenyl)ethan-1-amine hydrochloride. The compound was purified by column chromatography over silica gel using 25% ethyl acetate in hexane and isolated as colourless oil (250 mg, 55% yield). ¹H NMR (400 MHz, Chloroform-d): δ 9.06 (d, J=1.8 Hz, 1H), 8.84 (d, J=1.3 Hz, 1H), 8.08 (s, 1H), 7.31 (d, J=8.1 Hz, 1H), 7.09 (d, J=7.4 Hz, 1H), 6.92 (d, J=7.5 Hz, 1H), 6.79 (d, J=8.3 Hz, 1H), 6.31 (d, J=9.8 Hz, 1H), 5.30-5.20 (m, 1H), 3.82 (s, 3H).

5-Cyano-N-ethyl-N-(2,2,2-trifluoro-1-(2-methoxyphenyl)ethyl)pyridine-3-sulfonamide (Example 107): The ethylation of intermediate 53.1 was performed following the protocol as described in Method D at 60° C. using Cs₂CO₃. The crude was purified by column chromatography over silica gel using 20% ethyl acetate in hexane and the desired compound Example 107 was isolated as a light-yellow sticky gum (85 mg, 32% yield, 97.75% purity). LCMS: m/z found 400.17 [M+H]⁺, rt=3.14 min (Method 2) [Waters Acquity BEH C8 column (1.7 μm, 50×2.1 mm)]; ¹H NMR (400 MHz, Chloroform-d): δ 9.16 (d, J=2.0 Hz, 1H), 8.95 (d, J=1.5 Hz, 1H), 8.20 (t, J=2.1 Hz, 1H), 7.50 (d, J=7.7 Hz, 1H), 7.47-7.37 (m, 1H), 7.01 (t, J=7.7 Hz, 1H), 6.92-6.87 (m, 1H), 6.17 (q, J=8.8 Hz, 1H), 3.88 (s, 3H), 3.40-3.20 (m, 2H), 0.89 (t, J=7.0 Hz, 3H).

### (S)-5-Cyano-N-ethyl-N-(2,2,2-trifluoro-1-(2-methoxyphenyl)ethyl)pyridine-3-sulfonamide (Example 108 (EN-1)) and (R)-5-cyano-N-ethyl-N-(2,2,2-trifluoro-1-(2-methoxyphenyl)ethyl)pyridine-3-sulfonamide (Example 109

Chiral separation of racemic Example 107 provided both the enantiomers as mentioned below. Chiral separation method: Chiral separation was done on Thar SFC-80 series instrument by using CHIRALPAK IG column (21 mm×250 mm), 5μ, operating at 35° C. temperature, maintaining a flow rate of 40 mL/min, using 80% CO₂ in super critical state and 20% of 0.2% isopropylpamine in hexane/ethanol [80/20] as a mobile phase, this isocratic mixture run up to 10 min maintaining the isobaric condition of 100 bar at 278 nm wavelength.

Example 108: Off white solid (95.32% purity). LCMS: m/z found 400.3 [M+H]⁺, rt=3.39 min (Method 2) [Waters Acquity BEH C18 column (1.7 μm, 50×2.1 mm)]; ¹H NMR (400 MHz, Chloroform-d): δ 9.16 (d, J=2.0 Hz, 1H), 8.95 (d, J=1.7 Hz, 1H), 8.20 (t, J=2.0 Hz, 1H), 7.51 (d, J=7.8 Hz, 1H), 7.47-7.37 (m, 1H), 7.01 (td, J=1.2, 7.6 Hz, 1H), 6.89 (d, J=8.3 Hz, 1H), 6.17 (q, J=8.7 Hz, 1H), 3.86 (s, 3H), 3.40-3.21 (m, 2H), 0.89 (t, J=7.0 Hz, 3H).

Example 109: Off white solid (97.47% purity). LCMS: m/z found 400.3 [M+H]⁺, rt=3.39 min (Method 2) [Waters Acquity BEH C18 column (1.7 μm, 50×2.1 mm)]; ¹H NMR (400 MHz, Chloroform-d): δ 9.16 (d, J=1.8 Hz, 1H), 8.95 (d, J=1.6 Hz, 1H), 8.20 (t, J=1.9 Hz, 1H), 7.51 (d, J=7.8 Hz, 1H), 7.47-7.37 (m, 1H), 7.06-6.97 (m, 1H), 6.90 (d, J=8.3 Hz, 1H), 6.17 (q, J=8.7 Hz, 1H), 3.86 (s, 3H), 3.40-3.21 (m, 2H), 0.89 (t, J=7.0 Hz, 3H).

### Example 110 to Example 112

### 5-Cyano-N-ethyl-N-(2,2,2-trifluoro-1-(3-fluorophenyl)ethyl)pyridine-3-sulfonamide (Example 110), (R)-5-cyano-N-ethyl-N-(2,2,2-trifluoro-1-(3-fluorophenyl)ethyl)pyridine-3-sulfonamide (Example 111 (EN-1)) and (R)-5-cyano-N-ethyl-N-(2,2,2-trifluoro-1-(3-fluorophenyl)ethyl)pyridine-3-sulfonamide (Example 112 (EN-2

### 2,2,2-trifluoro-1-(3-fluorophenyl)ethan-1-amine hydrochloride 1.3

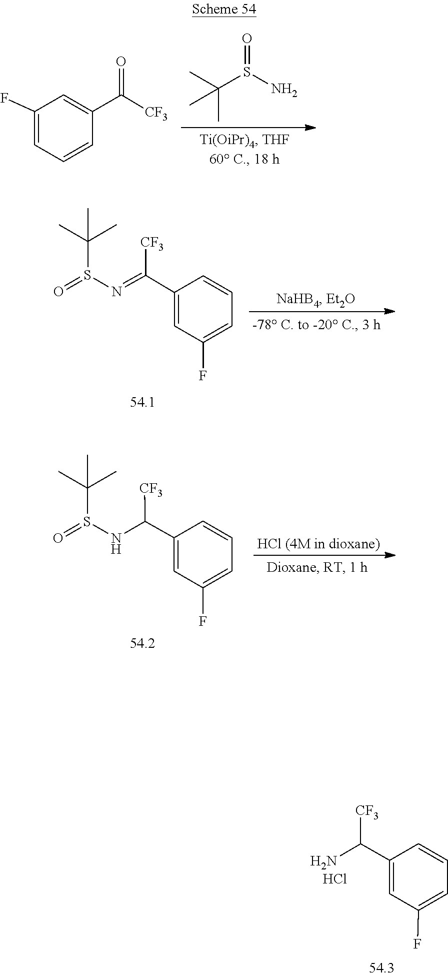

Synthesis of 2-methyl-N-(2,2,2-trifluoro-1-(3-fluorophenyl)ethylidene)propane-2-sulfinamide 54.1: Imine formation; Method P-I: To a stirred solution of 2-methylpropane-2-sulfinamide (695 mg, 5.726 mmol) in THF (10 mL) Ti(OⁱPr)₄ (4 mL, 13.01 mmol) was added and the RM was stirred for 10 mins at RT. 2,2,2-trifluoro-1-(3-fluorophenyl)ethan-1-one (1 g, 5.206 mmol) was added to it and the RM was heated at 80° C. overnight. The crude reaction mixture was forwarded to the following reduction step without any work up and purification.

Synthesis of 2-Methyl-N-(2,2,2-trifluoro-1-(3-fluorophenyl)ethylidene)propane-2-sulfinamide 54.2: The reaction mixture of the previous step was cooled at −78° C. and NaBH₄ (580 mg, 15.24 mmol) was added portion wise to the RM while maintaining the same temperature. The mixture was then warmed at −20° C. and stirred for 3 h, then poured gradually on an ice cold saturated NaCl solution with vigorous stirring. The resulting suspension was extracted with ethyl acetate (4×30 mL) and the organic part was then filtered through a plug of celite. The filtrate was further washed with saturated NaCl solution (30 mL) and dried (anhydrous Na₂SO₄). The volatiles were evaporated under reduced pressure and the crude product was purified by column chromatography over silica gel using 40% ethyl acetate in hexane to obtain the intermediate 12.2 as a light-yellow gum (900 mg, 60% yield). ¹H NMR (400 MHz, DMSO-d6): δ 7.50-7.39 (m, 3H), 7.22 (t, J=9.4 Hz, 1H), 6.70 (d, J=10.1 Hz, 1H), 5.30-5.20 (m, 1H), 1.05 (s, 9H).

Synthesis of 2,2,2-Trifluoro-1-(3-fluorophenyl)ethan-1-amine hydrochloride 54.3: Intermediate 54.2 (900 mg) was dissolved in 1,4-dioxane (5 ml) and cooled. 4 M HCl in dioxane (2 mL) was added to it and the RM was stirred at RT for 1 h. The volatiles were evaporated under reduced pressure and the crude compound 54.3 was used in the forwarding step without further purification.

### 5-Cyano-N-ethyl-N-(2,2,2-trifluoro-1-(3-fluorophenyl)ethyl)pyridine-3-sulfonamide (Example 110)

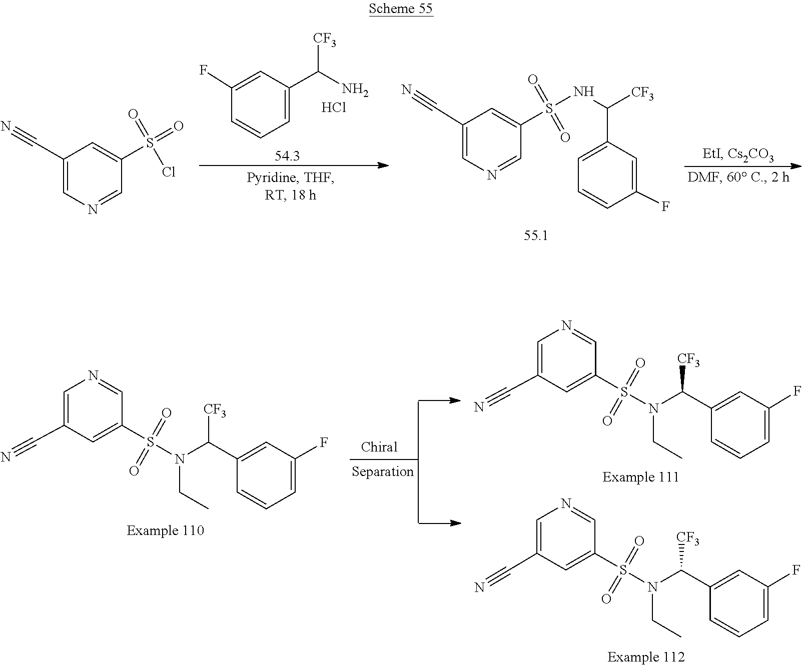

Synthesis of 5-cyano-N-(2,2,2-trifluoro-1-(3-fluorophenyl)ethyl)pyridine-3-sulfonamide 55.1: Intermediate 55.1 was synthesized from 5-cyanopyridine-3-sulfonyl chloride following the protocol as described in Method O using 2,2,2-trifluoro-1-(3-fluorophenyl)ethan-1-amine hydrochloride. The compound was purified by column chromatography over silica gel using 25% ethyl acetate in hexane and isolated as a colourless gum (450 mg, 62% yield). ¹H NMR (400 MHz, DMSO-d6): δ 9.90-9.75 (m, 1H), 9.13 (d, J=1.72 Hz, 1H), 9.01 (d, J=2.1 Hz, 1H), 8.47 (d, J=2.0 Hz, 1H) 7.40-7.20 (m, 3H), 7.20-7.10 (m, 1H), 5.60-5.50 (m, 1H).

Synthesis of 5-cyano-N-ethyl-N-(2,2,2-trifluoro-1-(3-fluorophenyl)ethyl)pyridine-3-sulfonamide (Example 110): The ethylation of intermediate 55.1 was performed following the protocol as described in Method D at 60° C. using Cs₂CO₃. The crude residue was purified by column chromatography over silica gel using 20% ethyl acetate in hexane and the desired compound Example 110 was isolated as a light-yellow sticky gum (150 mg, 30% yield, 99.80% purity). LCMS: m/z found 388.2 [M+H]⁺, rt=3.11 min (Method 2) [Waters Acquity BEH C18 column (1.7 μm, 50×2.1 mm)]; ¹H NMR (400 MHz, DMSO-d₆): δ 9.33 (dd, J=2.1, 13.3 Hz, 2H), 8.92 (t, J=2.0 Hz, 1H), 7.53-7.42 (m, 1H), 7.35-7.22 (m, 2H), 7.17 (d, J=10.1 Hz, 1H), 6.10 (q, J=8.4 Hz, 1H), 3.59-3.44 (m, 1H), 3.44-3.28 (m, 1H), 1.01 (t, J=7.0 Hz, 3H).

### (R)-5-Cyano-N-ethyl-N-(2,2,2-trifluoro-1-(3-fluorophenyl)ethyl)pyridine-3-sulfonamide (Example 111 (EN-1)) and (S)-5-cyano-N-ethyl-N-(2,2,2-trifluoro-1-(3-fluorophenyl)ethyl)pyridine-3-sulfonamide (Example 112 (EN-2))

Chiral separation of racemic Example 110 provided both the enantiomers as described below. Chiral separation method: chiral separation was done on an Agilent 1200 series instrument. Column name: CHIRALPAK IA (250×20 mm) 5u, operating at ambient temperature and at a flow rate is 18.0 mL/min. Mobile phase was a mixture of 80% Hexane, 10% EtOH, 10% Ethyl Acetate and 0.1% isopropylamine, holding this isocratic mixture to run up to 15 min with a wavelength of 246 nm.

Example 111: Light-yellow sticky gum (99.89% purity). LCMS: m/z found 388.2 [M+H]⁺, rt=3.26 min (Method 2) [Waters Acquity BEH C18 column (1.7 μm, 50×2.1 mm)]; ¹H NMR (400 MHz, DMSO-d₆): δ 9.41-9.23 (m, 2H), 8.92 (s, 1H), 7.48 (q, J=7.7 Hz, 1H), 7.36-7.22 (m, 2H), 7.18 (d, J=10.4 Hz, 1H), 6.10 (q, J=8.4 Hz, 1H), 3.56-3.46 (m, 1H), 3.42-3.30 (m, 1H), 1.01 (t, J=7.0 Hz, 3H).

Example 112: Light-yellow sticky gum (99.74% purity). LCMS: m/z found 388.2 [M+H]⁺, rt=3.26 min (Method 2) [Waters Acquity BEH C18 column (1.7 μm, 50×2.1 mm)]; ¹H NMR (400 MHz, DMSO-d₆): δ 9.33 (dd, J=2.0, 13.3 Hz, 2H), 8.92 (t, J=1.9 Hz, 1H), 7.53-7.42 (m, 1H), 7.35-7.22 (m, 2H), 7.18 (d, J=10.2 Hz, 1H), 6.10 (q, J=8.6 Hz, 1H), 3.56-3.44 (m, 1H), 3.44-3.31 (m, 1H), 1.00 (t, J=6.9 Hz, 3H).

### Example 113 to Example 115

### 5-Cyano-N-ethyl-N-(2,2,2-trifluoro-1-(m-tolyl)ethyl)pyridine-3-sulfonamide (Example 113), (R)-5-cyano-N-ethyl-N-(2,2,2-trifluoro-1-(m-tolyl)ethyl)pyridine-3-sulfonamide (Example 114), (S)-5-cyano-N-ethyl-N-(2,2,2-trifluoro-1-(m-tolyl)ethyl)pyridine-3-sulfonamide (Example 115)

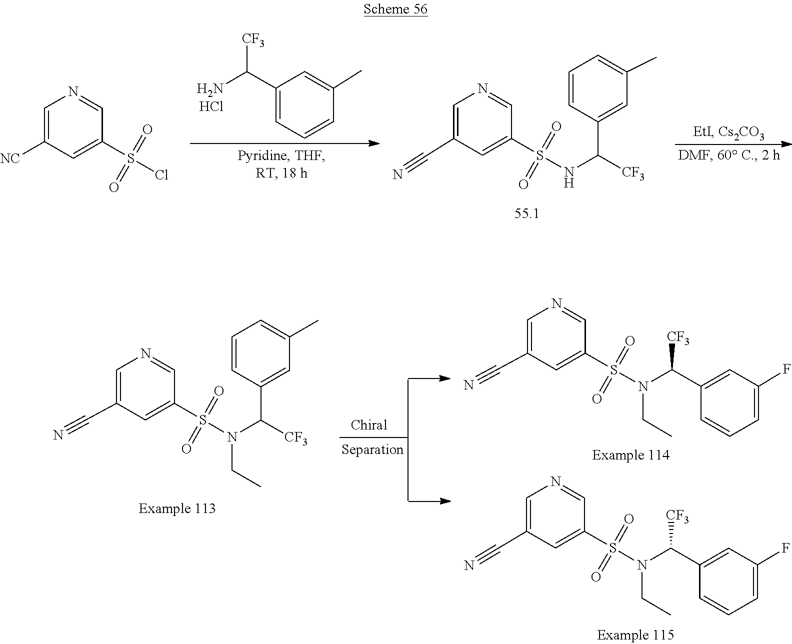

Synthesis of 5-cyano-N-(2,2,2-trifluoro-1-(m-tolyl)ethyl)pyridine-3-sulfonamide 56.1: Intermediate 56.1 was synthesized from 5-cyanopyridine-3-sulfonyl chloride following the protocol as described in Method O using 2,2,2-trifluoro-1-(m-tolyl)ethan-1-amine hydrochloride. The compound was purified by column chromatography over silica gel using 25% ethyl acetate in hexane and isolated as a colourless gum (450 mg, 62% yield). ¹H NMR (400 MHz, DMSO-d₆): δ 9.70-9.60 (m, 1H), 9.09 (d, J=1.8 Hz, 1H), 8.98 (d, J=2.0 Hz, 1H), 8.36 (d, J=1.9 Hz, 1H), 7.20-7.0 (m, 4H), 5.35-5.30 (m, 1H), 2.19 (s, 3H).

Synthesis of 5-cyano-N-ethyl-N-(2,2,2-trifluoro-1-(m-tolyl)ethyl)pyridine-3-sulfonamide (Example 113): The ethylation of intermediate 56.1 was performed following the protocol as described in Method D at 60° C. using Cs₂CO₃. The crude residue was purified by column chromatography over silica gel using 20% ethyl acetate in hexane and the desired compound Example 113 was isolated as a light-yellow sticky gum (145 mg, 29% yield, 99.89% purity). LCMS: m/z found 384.2 [M+H]⁺, rt=3.69 min (Method 2) [Waters Acquity BEH C18 column (1.7 μm, 50×2.1 mm)]; ¹H NMR (400 MHz, DMSO-d₆) δ 9.31 (dd, J=2.1, 8.6 Hz, 2H), 8.89 (t, J=2.0 Hz, 1H), 7.33-7.15 (m, 3H), 7.06 (s, 1H), 5.95 (q, J=8.6 Hz, 1H), 3.52-3.38 (m, 1H), 3.38-3.24 (m, 1H), 2.23 (s, 3H), 1.02 (t, J=7.0 Hz, 3H).

### (R)-5-cyano-N-ethyl-N-(2,2,2-trifluoro-1-(m-tolyl)ethyl)pyridine-3-sulfonamide (Example 114 (EN-1)) and (S)-5-cyano-N-ethyl-N-(2,2,2-trifluoro-1-(m-tolyl)ethyl)pyridine-3-sulfonamide (Example 115 (EN-2))

Chiral separation of racemic Example 113 provided both the enantiomers as mentioned below. Chiral separation method: chiral separation was done on an Agilent 1200 series instrument. Column name: Chiralpak IG (4.6×250 mm), 5μ, operating at ambient temperature and at a flow rate is 1.0 ml/min. Mobile phase was a mixture of 70% Hexane, 30% EtOH and 0.1% isopropylamine, holding this isocratic mixture to run up to 15 min with a wavelength of 246 nm.

Example 114: Colorless sticky gum (98.77% purity). LCMS: m/z found 384.3 [M+H]⁺, rt=3.46 min (Method 2) [Waters Acquity BEH C18 column (1.7 μm, 50×2.1 mm)]; ¹H NMR (400 MHz, Chloroform-d): δ 9.22 (d, J=1.8 Hz, 1H), 9.01 (d, J=1.9 Hz, 1H), 8.31 (s, 1H), 7.35-7.20 (m, 4H), 5.73 (q, J=8.4 Hz, 1H), 3.47-3.33 (m, 1H), 3.34-3.20 (m, 1H), 2.37 (s, 3H), 0.97 (t, J=7.0 Hz, 3H).

Example 115: Colorless sticky gum (99.23% purity). LCMS: m/z found 384.3 [M+H]⁺, rt=3.46 min (Method 2) [Waters Acquity BEH C18 column (1.7 μm, 50×2.1 mm)]; ¹H NMR (400 MHz, Chloroform-d): δ 9.22 (d, J=1.3 Hz, 1H), 9.02 (d, J=1.3 Hz, 1H), 8.31 (s, 1H), 7.38-7.14 (m, 4H), 5.73 (q, J=8.5 Hz, 1H), 3.47-3.20 (m, 2H), 2.37 (s, 3H), 0.97 (t, J=7.0 Hz, 3H).

### Example 116 and Example 117

### (R)-5-Cyano-N-ethyl-N-(2,2,2-trifluoro-1-(2-fluorophenyl)ethyl)pyridine-3-sulfonamide (Example 116) and (S)-5-cyano-N-ethyl-N-(2,2,2-trifluoro-1-(2-fluorophenyl)ethyl)pyridine-3-sulfonamide (Example 117)

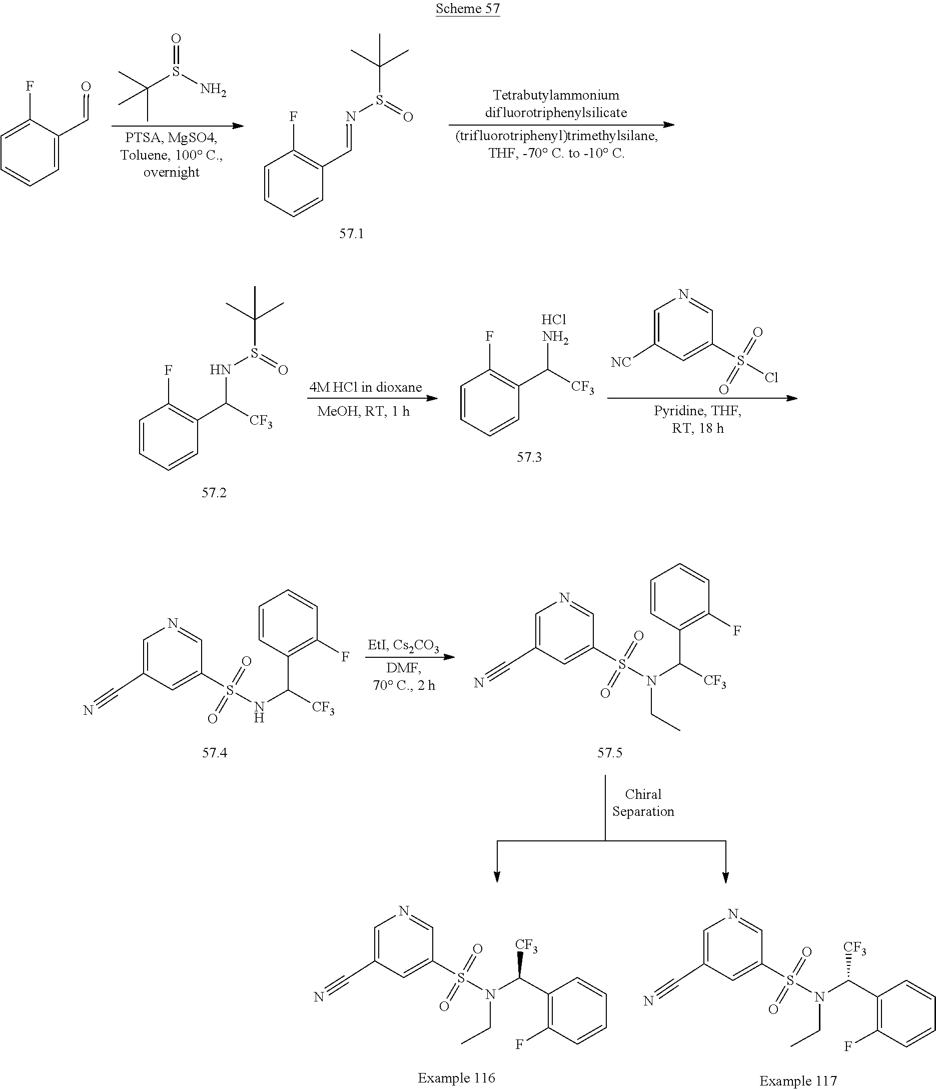

Synthesis of N-(2-fluorobenzylidene)-2-methylpropane-2-sulfinamide 57.1: To a stirred solution of 2-fluorobenzaldehyde (1 g, 5.20 mmol) and 2-methylpropane-2-sulfinamide (700 mg, 5.73 mmol) in toluene (10 mL), PTSA (100 mg, 0.52 mmol) was added followed by anhydrous MgSO₄. The reaction mixture was stirred at 80° C. for 16 h, then cooled to RT and the RM was filtered through celite. The volatiles were evaporated under reduced pressure and the crude was purified by CC over silica gel using 10% ethyl acetate in hexane to yield the desired compound as a yellow oil (400 mg, 34% yield). ¹H NMR (400 MHz, DMSO-d₆): 8.71 (s, 1H), 8.01 (t, J=7.4 Hz, 1H), 7.71-7.60 (m, 1H), 7.5-7.30 (m, 2H), 1.19 (s, 9H).

Synthesis of 2-methyl-N-(2,2,2-trifluoro-1-(2-fluorophenyl)ethyl)propane-2-sulfinamide 57.2: To a solution of intermediate 57.1 (200 mg, 0.88 mmol) in THF (2 mL) tetrabutylammonium difluorotriphenylsilicate (95 mg, 0.17 mmol) in THF (1 mL) at −70° C. was added and the RM was stirred for 30 min. CF₃TMS (0.4 mL, 2.64 mmol) was added dropwise at the same temperature and the RM was stirred for 4 h at −70° C., then warmed to −10° C. and stirred for further 4 h. The RM was quenched at −10° C. with aq. std. NH₄Cl solution (2 mL). It was then diluted with aq. std. NH₄Cl solution (10 mL) and extracted with ethyl acetate (2×20 mL). Combined organic layers were washed with water (10 mL) and brine (10 mL), dried, and the solvent was evaporated under reduced pressure. The crude residue was purified by CC over silica gel and compound 57.2 was isolated as a colourless gum (200 mg, yield 75%). ¹H NMR (400 MHz, Chloroform-d): δ 7.45-7.35 (m, 2H), 7.24-7.16 (m, 1H), 7.16-7.09 (m, 1H), 5.20-5.00 (m, 1H), 3.94-3.92 (m, 1H), 1.25 (s, 9H).

Synthesis of 2,2,2-trifluoro-1-(2-fluorophenyl)ethan-1-amine hydrochloride 57.3: Intermediate 57.2 (200 mg) was dissolved in 1,4-dioxane (2 ml) and cooled to 0° C. 4 M HCl in dioxane (1 mL) was added to it and the RM was stirred at RT for 1 h. The volatiles were evaporated under reduced pressure and the crude compound 57.3 was used in the following step without further purification.

Synthesis of 5-cyano-N-(2,2,2-trifluoro-1-(2-fluorophenyl)ethyl)pyridine-3-sulfonamide 57.4: Intermediate 57.4 was synthesized from 5-cyanopyridine-3-sulfonyl chloride following the protocol as described in Method O using 2,2,2-trifluoro-1-(2-fluorophenyl)ethan-1-amine hydrochloride. The compound was purified by column chromatography over silica gel using 25% ethyl acetate in hexane and isolated as a colourless gum (130 mg, 56% yield). ¹H NMR (400 MHz, DMSO-d₆): δ 10.02-9.95 (brs, 1H), 9.01 (s, 1H), 9.02 (s, 1H), 8.49 (s, 1H), 7.50-7.35 (m, 2H), 7.20-7.10 (m, 2H), 5.50-5.40 (m, 1H).

Synthesis of 5-cyano-N-ethyl-N-(2,2,2-trifluoro-1-(2-fluorophenyl)ethyl)pyridine-3-sulfonamide 57.5: The ethylation of intermediate 57.4 was performed following the protocol as described in Method D at 60° C. using Cs₂CO₃. The crude was purified by column chromatography over silica gel using 20% ethyl acetate in hexane and the desired compound 57.5 was isolated as a light-yellow sticky gum (50 mg, 36% yield). ¹H NMR (400 MHz, DMSO-d₆): δ 9.28 (s, 1H), 9.15 (s, 1H), 8.69 (s, 1H), 7.65-7.50 (m, 2H), 7.35-7.25 (m, 2H), 6.20-6.10 (m, 1H), 3.50-3.45 (m, 2H), 0.92 (t, J=6.8 MHz, 3H).

Chiral separation of racemic 57.5 provided both the enantiomers as mentioned below. Chiral separation method: chiral separation was done on THAR-SFC-80 instrument by using CHIRALPAK IG column (21 mm×25 cm), 5μ, operating at 35° C. temperature, maintaining a flow rate of 50 ml/min, using 90% CO₂ in super critical state and 10% of (METHANOL) as mobile phase, run this isocratic mixture up to 9 minutes and maintaining the isobaric condition of 100 bar at a 220 nm wavelength.

Example 116: Yellow sticky gum (96.15% purity). LCMS: m/z found 387.93 [M+H]⁺, rt=3.44 min (Method 14) [Waters Acquity BEH C18 column (1.7 μm, 50×2.1 mm)]; ¹H NMR (400 MHz, DMSO-d₆): δ 9.28 (s, 1H), 9.15 (s, 1H), 8.69 (s, 1H), 7.63 (t, J=7.7 Hz, 1H), 7.60-7.49 (m, 1H), 7.36-7.21 (m, 2H), 6.14 (q, J=8.3 Hz, 1H), 3.56-3.39 (m, 2H), 0.92 (t, J=6.8 Hz, 3H).

Example 117: Yellow sticky gum (98.86% purity). LCMS: m/z found 387.93 [M+H]⁺, rt=3.44 min (Method 14) [Waters Acquity BEH C18 column (1.7 μm, 50×2.1 mm)]; ¹H NMR (400 MHz, DMSO-d₆): δ 9.29 (s, 1H), 9.15 (s, 1H), 8.69 (s, 1H), 7.62 (t, J=7.1 Hz, 1H), 7.54 (q, J=7.1 Hz, 1H), 7.36-7.21 (m, 2H), 6.14 (q, J=8.6 Hz, 1H), 3.56-3.39 (m, 2H), 0.92 (t, J=6.8 Hz, 3H).

### Example 118 and Example 119

### (S)-5-Cyano-N-ethyl-N-(2,2,2-trifluoro-1-(4-(trifluoromethoxy)phenyl)ethyl)pyridine-3-sulfonamide (Example 118) and (R)-5-Cyano-N-ethyl-N-(2,2,2-trifluoro-1-(4-(trifluoromethoxy)phenyl)ethyl)pyridine-3-sulfonamide (Example 119)

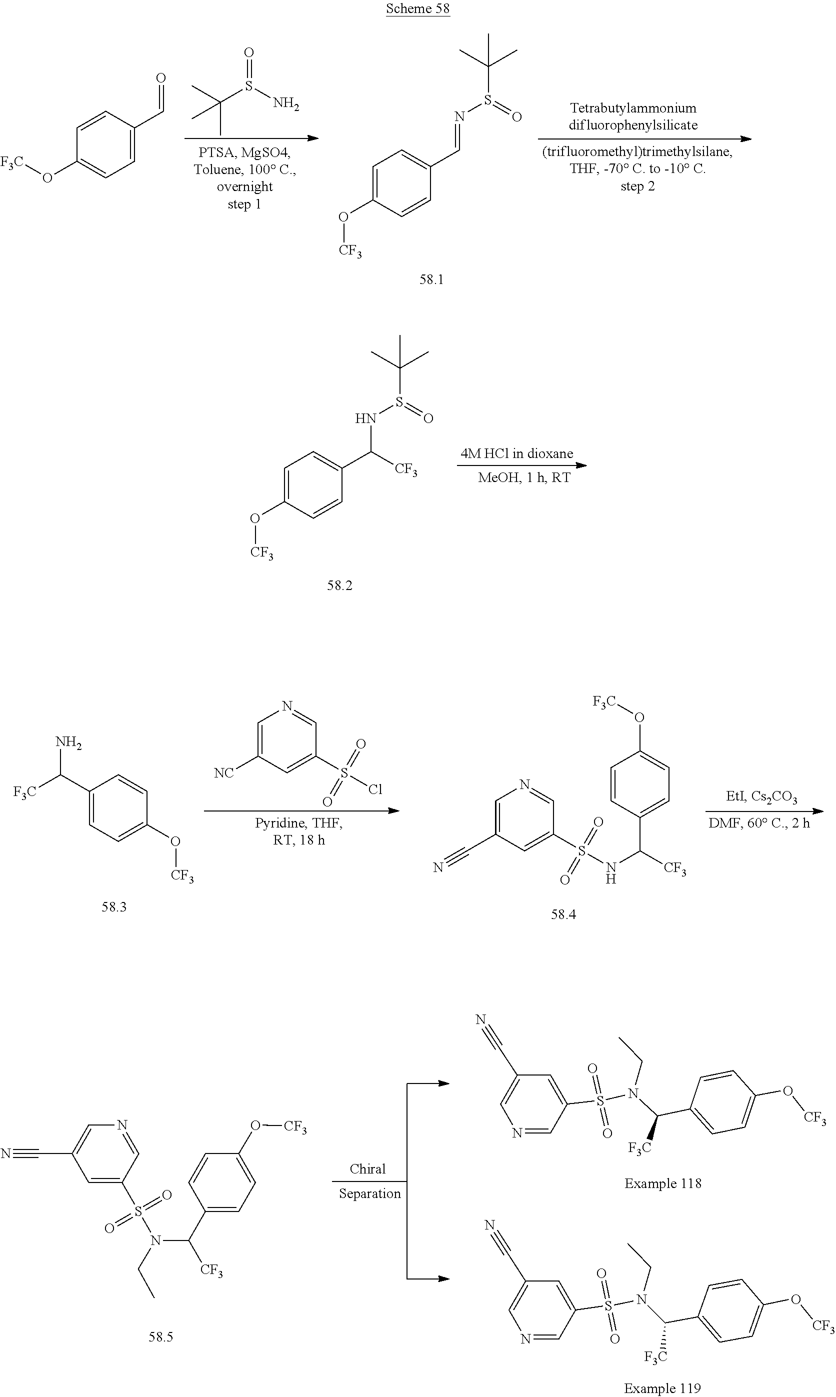

Synthesis of 2-methyl-N-(4-(trifluoromethoxy)benzylidene)propane-2-sulfinamide 58.1: To a stirred solution of 4-(trifluoromethoxy)benzaldehyde (2 g, 10.52 mmol) and 2-methylpropane-2-sulfinamide (1.53 g, 12.62 mmol) in toluene (20 mL) PTSA (100 mg, 0.53 mmol) was added followed by anhydrous MgSO₄. The reaction mixture was stirred at 80° C. for 16 h, then cooled to RT and the RM was filtered through celite. The volatiles were evaporated under reduced pressure and the crude was purified by CC over silica gel using 10% ethyl acetate in hexane to yield the desired compound as a yellow oil (2.5 g, 81% yield). ¹H NMR (400 MHz, DMSO-d₆): 8.59 (s, 1H), 8.08 (d, J=8.6 Hz, 2H), 7.53 (d, J=8.1 Hz, 2H), 1.18 (s, 9H).

Synthesis of 2-methyl-N-(2,2,2-trifluoro-1-(4-(trifluoromethoxy)phenyl)ethyl)propane-2-sulfinamide 58.2: To a solution of intermediate 58.1 (2 g, 6.819 mmol) in THF (11 mL) tetrabutylammonium difluorotriphenylsilicate (1.364 g, 1.364 mmol) in THF (11 mL) was added at −70° C. and the RM was stirred for 30 min. CF₃TMS (10 mL, 68.19 mmol) was added dropwise at the same temperature and the RM was stirred for 4 h at −70° C. It was then warmed to −10° C. and stirred for another 4 h. The RM was quenched at −10° C. with aq. std. NH4Cl solution (10 mL). It was then diluted with aq. std. NH4Cl solution (20 mL) and extracted with ethyl acetate (2×30 mL). The combined organic layers were washed with water (20 mL) and brine (20 mL), dried, and the solvent was evaporated under reduced pressure. The crude residue was purified by CC over silica gel and compound 358.2 was isolated as a colourless gum (1.6 g, yield 65%). ¹H NMR (400 MHz, DMSO-d₆): δ 7.75 (d, J=8.5 Hz, 2H), 7.44 (d, J=8.3 Hz, 2H), 6.48 (d, J=9.5 Hz, 1H), 5.40-5.30 (m, 1H), 1.14 (s, 9H).

Synthesis of 2,2,2-trifluoro-1-(4-(trifluoromethoxy)phenyl)ethan-1-amine hydrochloride 58.3: Intermediate 58.2 (200 mg) was dissolved in 1,4-dioxane (2 ml) and cooled. 4 M HCl in dioxane (1 mL) was added to it and the RM was stirred at RT for 1 h. The volatiles were evaporated under reduced pressure and the crude compound 58.3 was used in the forwarding step without further purification.

Synthesis of 5-cyano-N-(2,2,2-trifluoro-1-(4-(trifluoromethoxy)phenyl)ethyl)pyridine-3-sulfonamide 58.4: Intermediate 58.4 was synthesized from 5-cyanopyridine-3-sulfonyl chloride following the protocol as described in Method O using 2,2,2-trifluoro-1-(4-(trifluoromethoxy)phenyl)ethan-1-amine hydrochloride. The compound was purified by column chromatography over silica gel using 25% ethyl acetate in hexane and isolated as a colourless gum (600 mg, 57% yield). ¹H NMR (400 MHz, DMSO-d₆): δ 9.90-8.85 (brs, 1H), 9.10 (s, 1H), 9.01-9.0 (m, 1H), 8.44 (s, 1H), 7.53 (d, J=8.5 Hz, 2H), 7.28 (d, J=8.3 Hz, 2H), 5.70-5.50 (m, 1H).

Synthesis of 5-cyano-N-ethyl-N-(2,2,2-trifluoro-1-(4-(trifluoromethoxy)phenyl)ethyl)pyridine-3-sulfonamide 58.5: The ethylation of intermediate 58.4 was preformed following the protocol as described in Method D at 60° C. using Cs₂CO₃. The crude was purified by column chromatography over silica gel using 20% ethyl acetate in hexane and the desired compound 58.5 was isolated as a light-yellow sticky gum (418 mg, 52% yield). ¹H NMR (400 MHz, DMSO-d₆): δ 9.31 (d, J=10.8 Hz, 2H), 8.92 (s, 1H), 7.53 (d, J=8.1 Hz, 2H), 7.42 (d, J=8.3 Hz, 2H), 6.20-6.10 (m, 1H), 3.55-3.30 (m, 2H), 1.0 (t, J=6.6 MHz, 3H).

Chiral separation of racemic 58.5 provided both the enantiomers as mentioned below. Chiral separation method: chiral separation was done on THAR-SFC-80 instrument by using CHIRALPAK IG column (21 mm×25 cm), 5μ, operating at 35° C. temperature, maintaining a flow rate of 40 ml/min, using 90% C02 in super critical state and 10% methanol as mobile phase, this isocratic mixture run up to 9 minutes maintaining the isobaric condition of 100 bar at a 220 nm wavelength.

Example 118: Yellow sticky gum (98.36% purity). LCMS: m/z found 454.2 [M+H]⁺, rt=3.53 min (Method 2) [Waters Acquity BEH C18 column (1.7 μm, 50×2.1 mm)]; ¹H NMR (400 MHz, Chloroform-d) δ 9.25 (d, J=2.3 Hz, 1H), 9.05 (d, J=1.9 Hz, 1H), 8.39 (s, 1H), 7.56 (d, J=8.4 Hz, 2H), 7.29 (d, J=8.3 Hz, 2H), 5.82 (q, J=8.0 Hz, 1H), 3.50-3.36 (m, 1H), 3.28-3.14 (m, 1H), 0.94 (t, J=6.8 Hz, 3H).

Example 119: Yellow sticky gum (99.07% purity). LCMS: m/z found 454.2 [M+H]⁺, rt=3.53 min (Method 2) [Waters Acquity BEH C18 column (1.7 μm, 50×2.1 mm)]; ¹H NMR (400 MHz, Chloroform-d) δ 9.25 (d, J=2.3 Hz, 1H), 9.06 (d, J=1.9 Hz, 1H), 8.39 (s, 1H), 7.56 (d, J=8.4 Hz, 2H), 7.29 (d, J=8.4 Hz, 2H), 5.81 (q, J=8.3 Hz, 1H), 3.50-3.36 (m, 1H), 3.28-3.16 (m, 1H), 0.94 (t, J=7.2 Hz, 3H).

### Example 120

### 5-acetyl-N-ethyl-N-(2,2,2-trifluoro-1-(4-fluorophenyl)ethyl)pyridine-3-sulfonamide

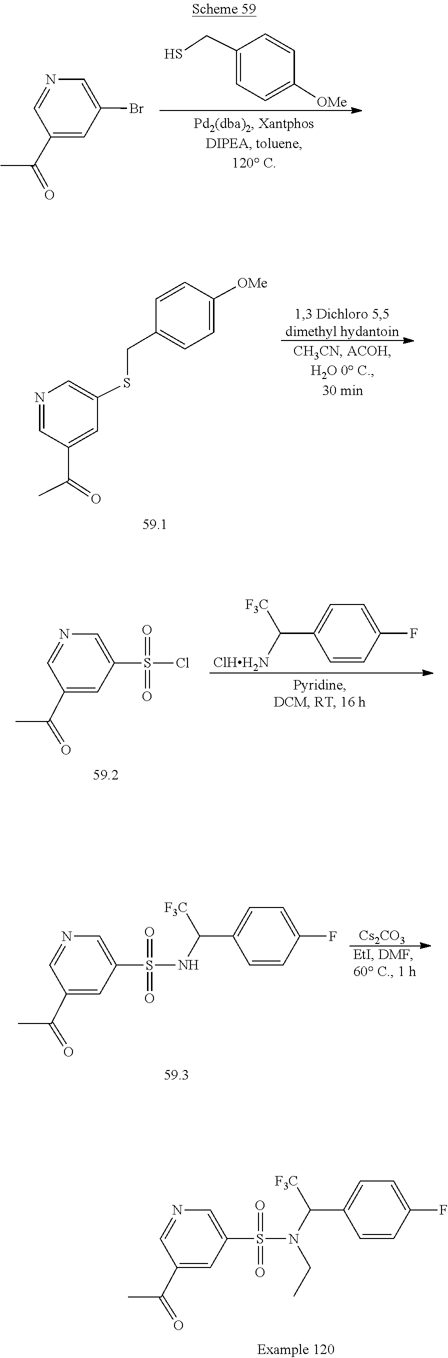

Synthesis of 1-(5-((4-methoxybenzyl)thio)pyridin-3-yl)ethan-1-one 59.1: Intermediate 59.1 was synthesized from 1-(5-bromopyridin-3-yl)ethan-1-one following a method analogous to that described in Method I using (4-methoxyphenyl)methanethiol. The crude was purified by column chromatography over silica gel using 20% ethyl acetate in hexane and compound 59.1 (2.5 g, 61% yield) as a yellow gum. LCMS: m/z found 274.3 [M+H]⁺, rt=2.05 min (Method 25) [Waters Acquity BEH C18 column (1.7 μm, 50×2.1 mm)].

Synthesis of 5-acetylpyridine-3-sulfonyl chloride 59.2: Sulfonyl chloride 59.2 was synthesized from intermediate 59.1 following the procedure described in Method T. The crude sulfonyl chloride was used immediately in the following step without further purification.

Synthesis of 5-acetyl-N-(2,2,2-trifluoro-1-(4-fluorophenyl)ethyl)pyridine-3-sulfonamide 59.3: Intermediate 59.3 was synthesized from 5-cyanopyridine-3-sulfonyl chloride following the protocol as described in Method O using 2,2,2-trifluoro-1-(4-(trifluoromethyl)phenyl)ethan-1-amine hydrochloride. The compound was purified by column chromatography over silica gel using 35% ethyl acetate in hexane and isolated as a yellow sticky gum (200 mg, 19% yield). LCMS: m/z found (376.85 [M+H]⁺), rt=1.85 min (Method 1) [Waters Acquity BEH C18 column (1.7 μm, 50×2.1 mm)].

Synthesis of 5-acetyl-N-ethyl-N-(2,2,2-trifluoro-1-(4-fluorophenyl)ethyl)pyridine-3-sulfonamide (Example 120): The ethylation of intermediate 59.3 was performed following the protocol as described in Method D at 60° C. The crude was purified by Reverse Phase (RP) Preparative HPLC and the compound was obtained as white solid (10 mg, 99.62% purity).

RP method: preparative HPLC was done on a WATERS auto purification instrument. Column name: YMC-Actus Triart C18 (250×20 mm, 5μ), operating at ambient temperature and at a flow rate of 16.0 ml/min. Mobile phase: A=20 mM ammonium bicarbonate in water, B=Acetonitrile; Gradient Profile: Mobile phase initial composition of 80% A and 20% B, then to 50% A and 50% B in 3 min, then to 20% A and 80% B in 18 min, then to 5% A and 95% B in 18.5 min, held this composition up to 21.5 min for column washing, then returned to initial composition in 22 min and held till 25 min. LCMS: m/z found 405.17 [M+H]⁺, rt=3.44 min (Method 2) [Waters Acquity BEH C8 column (1.7 μm, 50×2.1 mm)]; ¹H NMR (400 MHz, DMSO-d₆): δ 9.37-9.26 (m, 2H), 8.59 (s, 1H), 7.47 (dd, J=5.1, 8.5 Hz, 2H), 7.24 (t, J=8.6 Hz, 2H), 6.16 (q, J=8.8 Hz, 1H), 3.53-3.33 (m, 2H), 2.70 (s, 3H), 0.98 (t, J=6.9 Hz, 3H).

### Example 121

### N-ethyl-6-methoxy-N-(2,2,2-trifluoro-1-(4-fluorophenyl)ethyl)pyridazine-4-sulfonamide

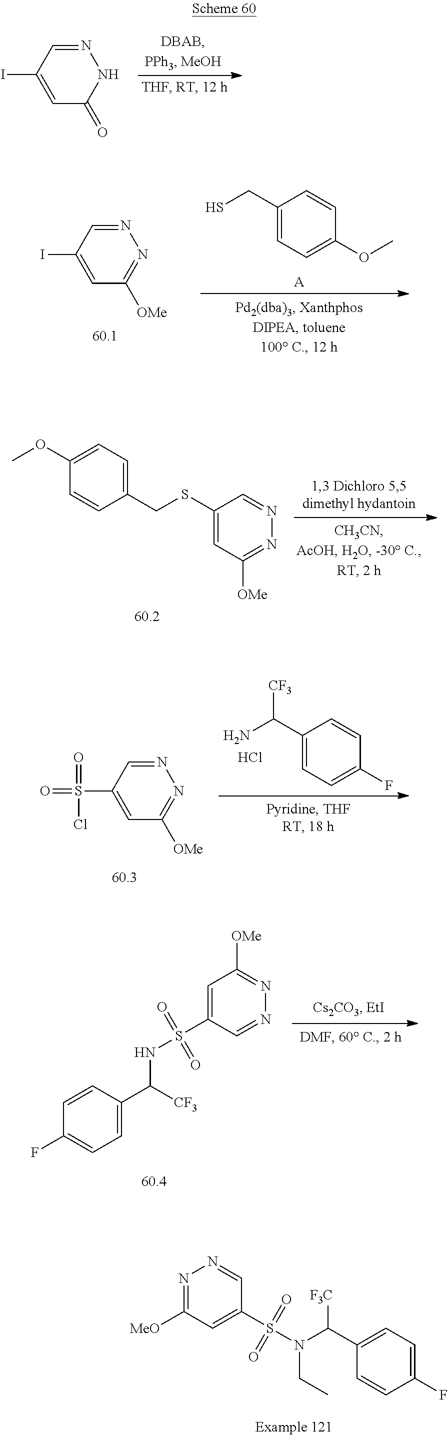

Synthesis of 5-iodo-3-methoxypyridazine 60.1: To a solution of the 5-iodopyridazin-3(2H)-one (3 g, 13.57 mmol) in THF (100 mL) PPh₃ (7.1 g, 27.15 mmol) was added followed by MeOH (3 mL). The reaction mixture was cooled to 0° C. and DBAB (4.6 g, 20.36 mmol) was added to it portion-wise and stirred at RT for 12 h. It was then diluted with water (50 mL) and extracted with EtOAc (2×100 mL). The combined organic layers were washed with water (2×50 mL) and brine (50 mL), dried over anhydrous Na₂SO₄ and the solvent was evaporated under reduced pressure. The crude was purified by column chromatography over silica gel using 50% ethyl acetate-hexane and 5-iodo-3-methoxypyridazine was isolated as white solid (3.0 g, 93% yield). ¹H NMR (400 MHz, DMSO-d₆): δ 7.8 (s, 1H), 7.4 (s, 1H), 3.7 (s, 3H).

Synthesis of 3-methoxy-5-((4-methoxybenzyl)thio)pyridazine 60.2: Intermediate 60.2 was synthesized from intermediate 60.1 following a method analogous to that described in Method I using (4-methoxyphenyl)methanethiol. The crude residue was purified by column chromatography over silica gel using 20% ethyl acetate-hexane to get 3-methoxy-5-((4-methoxybenzyl)thio)pyridazine as a yellow gum (258 mg, 75% yield). ¹H NMR (400 MHz, DMSO-d₆): δ 7.78 (d, J=1.6 Hz, 1H), 7.36 (d, J=8.3 Hz, 2H), 6.90 (d, J=8.4 Hz, 2H), 6.72 (d, J=1.6 Hz, 1H), 4.30 (s, 2H), 3.73 (s, 3H), 3.56 (s, 3H).

Synthesis of 6-methoxypyridazine-4-sulfonyl chloride 60.3: Sulfonyl chloride 60.3 was synthesized from intermediate 60.2 following the procedure described in Method T. The crude sulfonyl chloride was used immediately in the following step without further purification.

Synthesis of 6-methoxy-N-(2,2,2-trifluoro-1-(4-fluorophenyl)ethyl)pyridazine-4-sulfonamide 60.4: Intermediate 60.4 was synthesized from sulfonyl chloride 60.3 following the protocol as described in Method O using 2,2,2-trifluoro-1-(4-fluorophenyl)ethan-1-amine hydrochloride. The compound was purified by column chromatography over silica gel using 35% ethyl acetate in hexane and intermediate 60.4 was isolated as a yellow gum (60 mg, 17% yield). ¹H NMR (400 MHz, DMSO-d₆): δ 9.98 (s, 1H), 7.96-7.90 (m, 1H), 7.58-7.49 (m, 2H), 7.19 (t, J=8.7 Hz, 2H), 7.03 (s, 1H), 5.67-5.30 (m, 1H), 3.58 (s, 3H).

Synthesis of N-ethyl-6-methoxy-N-(2,2,2-trifluoro-1-(4-fluorophenyl)ethyl)pyridazine-4-sulfonamide (Example 121): The ethylation of intermediate 60.4 was performed following the protocol as described in Method D using Cs₂CO₃ at 60° C. The compound was purified by column chromatography over silica gel using 35% ethyl acetate in hexane and Example 121 was isolated as a colorless sticky gum (12 mg, 98.42% purity, 25% yield). LCMS: m/z found (394.0 [M+H]⁺, rt=2.84 min (Method 9) [Xbridge C18 column (3.5 μm, 50×3 mm)]; ¹H NMR (400 MHz, DMSO-d₆): δ 8.30 (s, 1H), 7.55 (t, J=5.7 Hz, 2H), 7.42 (s, 1H), 7.30 (t, J=8.6 Hz, 2H), 6.09 (q, J=8.6 Hz, 1H), 3.69 (s, 3H), 3.52-3.35 (m, 2H), 0.98 (t, J=7.0 Hz, 3H).

### Example 122 and Example 123

### (S)—N-ethyl-6-methoxy-N-(2,2,2-trifluoro-1-(4-fluorophenyl)ethyl)pyridazine-4-sulfonamide (Example 122) and (R)—N-ethyl-6-methoxy-N-(2,2,2-trifluoro-1-(4-fluorophenyl)ethyl)pyridazine-4-sulfonamide (Example 123)

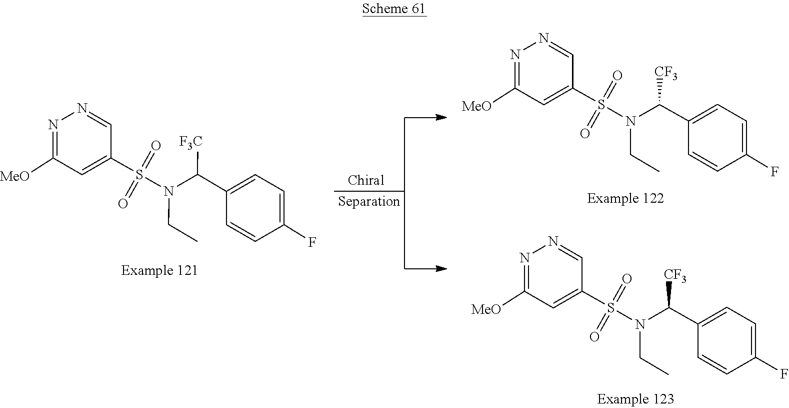

Chiral separation of racemic Example 121 provided both the enantiomers as mentioned below. Chiral separation method: viral separation was done on THAR-SFC-80 instrument by using CHIRALPAK IG column (21 mm×25 cm), 5μ, operating at 35° C. temperature, maintaining a flow rate of 40 ml/min, using 90% CO₂ in super critical state and 0.3% isopropylamine in methanol as a mobile phase, running this isocratic mixture up to 9 minutes and maintaining the isobaric condition of 100 bar at a 220 nm wavelength.

Example 122: Colourless sticky gum (99.85% purity). LCMS: m/z found 394.15 [M+H]⁺, rt=2.97 min (Method 2) [Waters Acquity BEH C8 column (1.7 μm, 50×2.1 mm)]; ¹H NMR (400 MHz, DMSO-d₆): δ 8.30 (d, J=2.0 Hz, 1H), 7.55 (dd, J=5.1, 8.7 Hz, 2H), 7.42 (d, J=2.0 Hz, 1H), 7.30 (t, J=8.8 Hz, 2H), 6.09 (q, J=8.6 Hz, 1H), 3.69 (s, 3H), 3.42 (qt, J=7.9, 15.5 Hz, 2H), 0.98 (t, J=6.9 Hz, 3H).

Example 123: Colourless sticky gum (98.85% purity). LCMS: m/z found 394.15 [M+H]⁺, rt=2.97 min (Method 2) [Waters Acquity BEH C8 column (1.7 μm, 50×2.1 mm)]; ¹H NMR (400 MHz, DMSO-d₆): δ 8.30 (d, J=2.1 Hz, 1H), 7.55 (dd, J=5.2, 8.7 Hz, 2H), 7.42 (d, J=2.0 Hz, 1H), 7.30 (t, J=8.8 Hz, 2H), 6.09 (q, J=8.6 Hz, 1H), 3.69 (s, 3H), 3.49-3.35 (m, 2H), 0.98 (d, J=7.0 Hz, 3H).

### Example 124 and Example 125

### N-ethyl-N-(2,2,2-trifluoro-1-(4-methoxyphenyl)ethyl)tetrahydro-2H-pyran-4-sulfonamide (Example 124) and N-methyl-N-(2,2,2-trifluoro-1-(4-methoxyphenyl)ethyl)tetrahydro-2H-pyran-4-sulfonamide (Example 125)

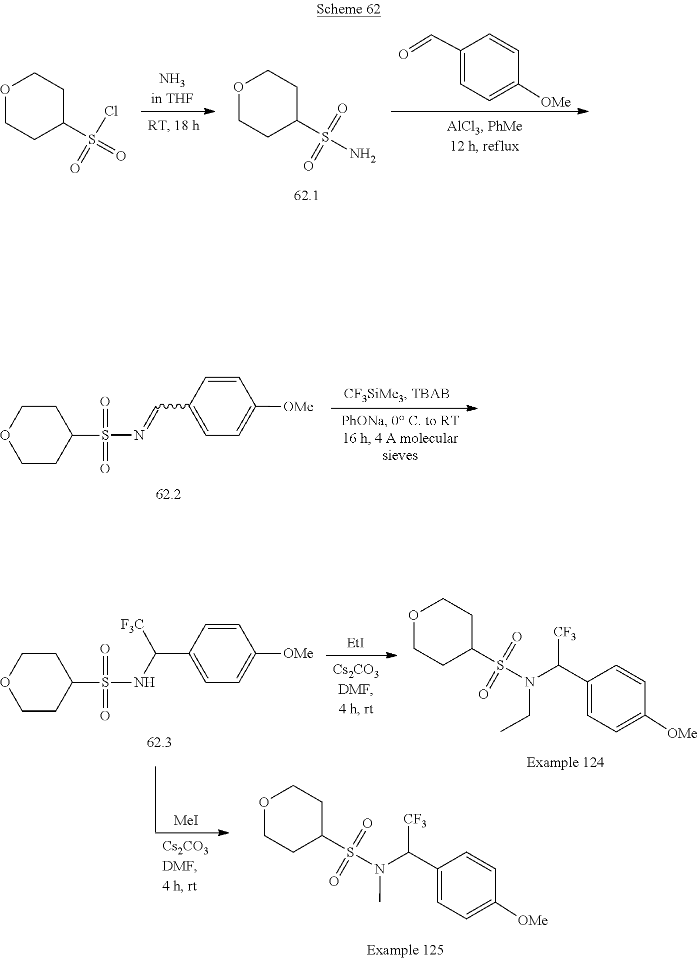

Synthesis of tetrahydro-2H-pyran-4-sulfonamide 62.1: To a stirred solution of tetrahydro-2H-pyran-3-sulfonyl chloride (400 mg, 2.16 mmol) in THF (1 mL), freshly prepared NH₃ in THF (20 mL) was added at 0° C. and the RM was stirred at room temperature for 16 h. The reaction was then filtered through a sintered funnel and the residue was collected. The residue was purified by column chromatography over silica gel using 5:1 ethyl acetate-hexane. Intermediate 62.1 was obtained as a white solid (200 mg, 56% yield). ¹H NMR (400 MHz, DMSO-d₆): δ 6.75 (s, 2H), 3.93 (dd, J=4.6, 11.5 Hz, 2H), 3.41-3.22 (m, 2H), 3.12-2.98 (m, 1H), 1.92-1.83 (m, 2H), 1.68-1.52 (m, 2H).

Synthesis of N-(4-methoxybenzylidene)tetrahydro-2H-pyran-4-sulfonamide 62.2: To a stirred solution of 62.1 (200 mg, 1.21 mmol) and 4-methoxybenzaldehyde (224 mg, 1.45 mmol) in toluene (2 mL) AlCl₃ (48 mg, 0.36 mmol) was added and the mixture was refluxed for 12 h. The reaction was then filtered through a sintered funnel and the residue was collected and directly used for the next step without further purification.

Synthesis of N-(2,2,2-trifluoro-1-(4-methoxyphenyl)ethyl)tetrahydro-2H-pyran-4-sulfonamide 62.3: To a stirred solution of 62.2 (300 mg, 1.10 mmol) in toluene (3 mL) under N₂ atmosphere, was added TBAB (21 mg, 0.06 mmol) followed by 4A molecular sieve. The RM was cooled to 0° C. and CF₃SiMe₃ (235 mg, 1.66 mmol) was added, followed by sodium phenoxide (282 mg, 1.66 mmol). The mixture was then stirred at RT for 12 h. The reaction was quenched with a saturated Na₂CO₃ solution (10 mL) and extracted with ethyl acetate (2×20 mL). Organic layers were collected filtered through silica and concentrated under reduced pressure. The crude residue was purified by column chromatography over silica gel using 1:5 ethyl acetate-hexane and the compound 62.3 was obtained as white solid (150 mg, 38% yield).

Synthesis of N-ethyl-N-(2,2,2-trifluoro-1-(4-methoxyphenyl)ethyl)tetrahydro-2H-pyran-4-sulfonamide (Example 124): The ethylation of intermediate 62.3 was performed following the protocol as described in Method D at RT. The crude residue was purified by column chromatography over silica gel using 1:5 ethyl acetate-hexane. Example 124 was obtained as white solid (70 mg, 42% yield, 98.9% purity). GC-MS (CHE 300-METHOD) (HP-5MS (30×250 μm×0.25 μm) m/z found 381.1; ¹H NMR (400 MHz, DMSO-de): δ 7.51 (d, J=8.4 Hz, 2H), 7.03 (d, J=8.6 Hz, 2H), 5.67 (q, J=8.8 Hz, 1H), 3.99-3.88 (m, 2H), 3.79 (s, 3H), 3.65-3.52 (m, 1H), 3.38-3.15 (m, 4H), 1.83-1.57 (m, 4H), 0.96 (t, J=7.0 Hz, 3H).

Synthesis of N-methyl-N-(2,2,2-trifluoro-1-(4-methoxyphenyl)ethyl)tetrahydro-2H-pyran-4-sulfonamide (Example 125): The methylation of intermediate 62.3 was performed following the protocol as described in Method D at RT. The crude residue was purified by column chromatography over silica gel using 1:5 ethyl acetate-hexane. Example 125 as obtained as a colorless oil (205 mg, 49% yield, 99.88% purity). GC-MS (CHE 300-METHOD) (HP-5MS (30×250 μm×0.25 μm) m/z found 381.1; ¹H NMR (400 MHz, Chloroform-d): δ 7.40 (d, J=8.7 Hz, 2H), 6.97-6.88 (m, 2H), 5.65 (q, J=8.7 Hz, 1H), 4.16-4.04 (m, 2H), 3.81 (s, 3H), 3.42-3.19 (m, 3H), 2.76 (s, 3H), 2.07-1.88 (m, 4H).

### Example 126 and Example 127

### (S)—N-ethyl-N-(2,2,2-trifluoro-1-(4-methoxyphenyl)ethyl)tetrahydro-2H-pyran-4-sulfonamide (Example 126) and (R)—N-ethyl-N-(2,2,2-trifluoro-1-(4-methoxyphenyl)ethyl)tetrahydro-2H-pyran-4-sulfonamide (Example 127)

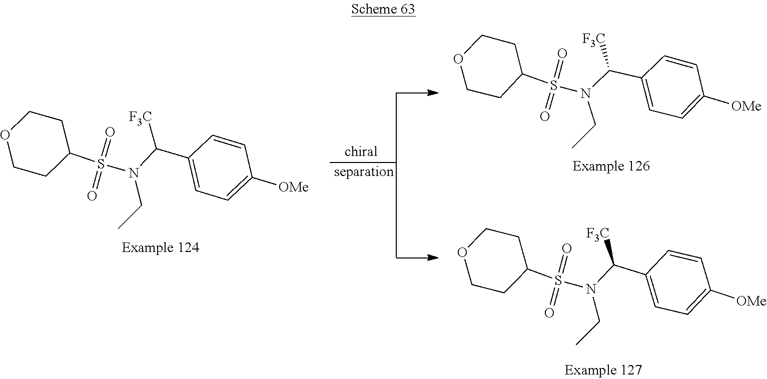

Chiral separation of racemic Example 124 provided both the enantiomers as described below. Chiral separation method: chiral separation was done on THAR-SFC-80 instrument by using CHIRALPAK IG column (21 mm×25 cm), 5μ, operating at 35° C. temperature, maintaining a flow rate of 60 ml/min, using 80% CO₂ in super critical state and 20% of (hexane/methanol/isopropanol [70/20/10]) as mobile phase, running this isocratic mixture up to 14 minutes and maintaining the isobaric condition of 100 bar at a 228 nm wavelength.

Example 126: White solid (18 mg, 98.79% purity). GC-MS (CHE 300H-METHOD) (HP-5MS (30×250 μm×0.25 μm) m/z found 381.1; ¹H NMR (400 MHz, Chloroform-d): δ 7.47 (d, J=8.3 Hz, 2H), 6.92 (d, J=8.6 Hz, 2H), 5.55 (q, J=8.6 Hz, 1H), 4.14-4.05 (m, 2H), 3.81 (s, 3H), 3.43-3.28 (m, 3H), 3.27-3.13 (m, 2H), 2.06-1.92 (m, 4H), 0.91 (t, J=7.0 Hz, 3H).

Example 127: White solid (17 mg, 94.06% purity). GC-MS (CHE 300H-METHOD) (HP-5MS (30×250 μm×0.25 μm) m/z found 381.1; ¹H NMR (400 MHz, Chloroform-d): δ 7.47 (d, J=8.4 Hz, 2H), 6.92 (d, J=8.7 Hz, 2H), 5.55 (q, J=8.4 Hz, 1H), 4.18-4.02 (m, 2H), 3.81 (s, 3H), 3.42-3.28 (m, 3H), 3.26-3.16 (m, 2H), 2.22-1.72 (m, 4H), 0.92 (t, J=7.0 Hz, 3H).

### Example 128 and Example 129

### (S)—N-methyl-N-(2,2,2-trifluoro-1-(4-methoxyphenyl)ethyl)tetrahydro-2H-pyran-4-sulfonamide (Example 128) and (R)—N-methyl-N-(2,2,2-trifluoro-1-(4-methoxyphenyl)ethyl)tetrahydro-2H-pyran-4-sulfonamide (Example 129)

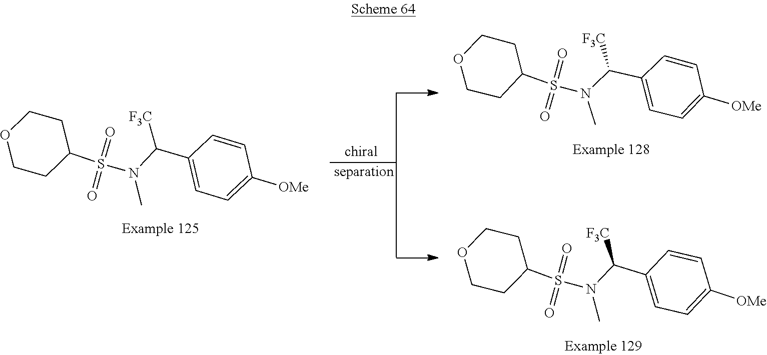

Chiral separation of racemic Example 125 provided both the enantiomers as mentioned below. Chiral separation method: chiral separation was done on a THAR-SFC-80 instrument by using a CHIRALPAK IG column (21 mm×25 cm), 5μ, operating at 35° C. temperature, maintaining flow rate of 50 ml/min, using 80% CO₂ in super critical state and 20% methanol as a mobile phase, running this isocratic mixture up to 12 minutes and maintaining the isobaric condition of 100 bar at a 220 nm wavelength.

Example 128: White solid (60 mg, 98.33% purity). GC-MS (CHE 300-METHOD) (HP-5MS (30×250 μm×0.25 μm) m/z found 367.1; ¹H NMR (400 MHz, Chloroform-d) δ 7.40 (d, J=8.4 Hz, 2H), 6.93 (d, J=8.7 Hz, 2H), 5.65 (q, J=8.5 Hz, 1H), 4.14-4.04 (m, 2H), 3.81 (s, 3H), 3.42-3.31 (m, 2H), 3.32-3.19 (m, 1H), 2.76 (s, 3H), 2.04-1.89 (m, 4H).

Example 129: White solid (62 mg, 99.95% purity). GC-MS (CHE 300-METHOD) (HP-5MS (30×250 μm×0.25 μm) m/z found 367.1; ¹H NMR (400 MHz, Chloroform-d) δ 7.40 (d, J=8.2 Hz, 2H), 6.93 (d, J=8.4 Hz, 2H), 5.65 (q, J=8.4 Hz, 1H), 4.13-4.05 (m, 2H), 3.81 (s, 3H), 3.42-3.31 (m, 2H), 3.31-3.11 (m, 1H), 2.76 (s, 3H), 2.03-1.89 (m, 4H).

### Example 130

### N-methyl-N-(2,2,2-trifluoro-1-(4-fluorophenyl)ethyl)tetrahydro-2H-pyran-4-sulfonamide

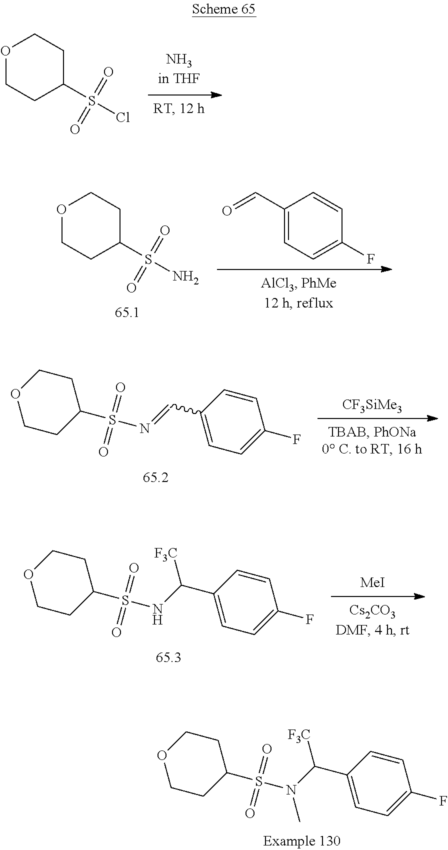

Synthesis of tetrahydro-2H-pyran-4-sulfonamide 65.1: To a stirred solution of in NH₃ in THF (15 mL), tetrahydro-2H-pyran-4-sulfonyl chloride (200 mg, 1.335 mmol) was added at 0° C. and the RM was stirred at RT for 12 h. The reaction was then concentrated to a residue. The residue was purified by column chromatography over silica gel using 1:1 ethyl acetate-hexane and the compound 65.1 was obtained as white solid (115 mg, 51% yield). ¹H NMR (400 MHz, DMSO-d₆) δ 6.75 (s, 2H), 3.93 (dd, J=4.6, 11.5 Hz, 2H), 3.41-3.22 (m, 2H), 3.12-2.98 (m, 1H), 1.92-1.83 (m, 2H), 1.68-1.52 (m, 2H).

Synthesis of N-(4-fluorobenzylidene)tetrahydro-2H-pyran-4-sulfonamide 65.2: To a stirred solution of intermediate 65.1 (350 mg, 2.12 mmol) and 4-fluorobenzaldehyde (289 mg, 2.33 mmol) in toluene (5 mL), AlCl₃ (113 mg, 0.84 mmol) was added and the RM was refluxed for 12 h. The reaction was then concentrated, and the residue was diluted with ethyl acetate (20 mL) and filtered. Then filtrate was concentrated and triturated with pentane:ether (1:1). The crude mass was directly used for next step without purification.

Synthesis of N-(2,2,2-trifluoro-1-(4-fluorophenyl)ethyl)tetrahydro-2H-pyran-4-sulfonamide 12.3: To a stirred solution of intermediate 65.2 (200 mg, 0.74 mmol) in toluene (4 mL) TBAB (14 mg, 0.04 mmol) and 4A molecular sieves were added under N₂ atmosphere. The RM was cooled at 0° C. and CF₃SiMe₃ (39 mg, 0.27 mmol) was added to it, followed by PhONa (128 mg, 1.10 mmol) and the mixture was stirred at RT for 16 h. Volatiles were evaporated under reduced pressure and the crude was diluted with satd. Na₂CO₃ (10 mL). The mixture was then extracted with EtOAc (2×10 mL), the combined organic layers were washed with water (10 mL) and brine (10 mL). The crude residue was purified by column chromatography over silica gel using 1:1 ethyl acetate-hexane and intermediate 65.3 was obtained as a colorless oil (140 mg, 55% yield). ¹H NMR (400 MHz, DMSO-d₆): δ 8.88 (d, J=9.9 Hz, 1H), 7.69 (dd, J=5.4, 8.5 Hz, 2H), 7.29 (t, J=8.8 Hz, 2H), 5.37-5.23 (m, 1H), 3.98-3.71 (m, 2H), 3.26-3.16 (m, 1H), 3.11-3.03 (m, 2H), 1.83-1.73 (m, 1H), 1.67-1.48 (m, 2H), 1.48-1.33 (m, 1H).

Synthesis of N-methyl-N-(2,2,2-trifluoro-1-(4-fluorophenyl)ethyl)tetrahydro-2H-pyran-4-sulfonamide (Example 130): The methylation of intermediate 65.3 was performed following the protocol as described in Method D at RT. The crude residue was purified by column chromatography over silica gel using 1:1 ethyl acetate-hexane. Example 130 was obtained as white solid (190 mg, 73% yield, 99.19% purity). GC-MS (CHE 300-METHOD) (HP-5MS (30×250 μm×0.25 μm) m/z found 355.1; ¹H NMR (400 MHz, Chloroform-d) δ 7.48 (dd, J=5.1, 8.5 Hz, 2H), 7.16-7.06 (m, 2H), 5.70 (q, J=8.5 Hz, 1H), 4.15-4.05 (m, 2H), 3.44-3.31 (m, 2H), 3.34-3.21 (m, 1H), 2.75 (s, 3H), 2.15-1.81 (m, 4H).

### Example 131 and Example 132

### (S)—N-methyl-N-(2,2,2-trifluoro-1-(4-fluorophenyl)ethyl)tetrahydro-2H-pyran-4-sulfonamide (Example 131) and (R)—N-methyl-N-(2,2,2-trifluoro-1-(4-fluorophenyl)ethyl)tetrahydro-2H-pyran-4-sulfonamide (Example 132)

Chiral separation of racemic Example 130 provided both the enantiomers as mentioned below. Chiral separation method: chiral separation was done on a THAR-SFC-80 instrument by using REFLECT C-AMYLOSE-A column (30 mm×25 cm), 5μ, operating at 35° C. temperature, maintaining a flow rate of 60 ml/min, using 65% CO₂ in super critical state and 35% methanol as a mobile phase, running this isocratic mixture up to 10 minutes and maintaining the isobaric condition of 100 bar at a 214 nm wavelength.

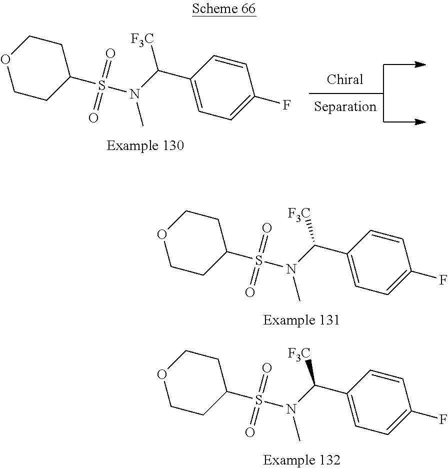 [SMILES: CN(C(c1ccc(F)cc1)C(F)(F)F)S(=O)(=O)C1CCOCC1 ; CN([C@@H](c1ccc(F)cc1)C(F)(F)F)S(=O)(=O)C1CCOCC1 ; CN([C@H](c1ccc(F)cc1)C(F)(F)F)S(=O)(=O)C1CCOCC1]

Example 131: White solid (72 mg, 98.28% purity). GC-MS (CHE 300H-METHOD) (HP-5MS (30×250 μm×0.25 μm) m/z found 355.1; ¹H NMR (400 MHz, Chloroform-d): δ 7.48 (dd, J=5.2, 8.5 Hz, 2H), 7.11 (t, J=8.6 Hz, 2H), 5.70 (q, J=8.5 Hz, 1H), 4.15-4.04 (m, 2H), 3.44-3.31 (m, 2H), 3.34-3.21 (m, 1H), 2.75 (s, 3H), 2.18-1.84 (m, 4H).

Example 132: White solid (70 mg, 99.80% purity). GC-MS (CHE 300-METHOD) (HP-5MS (30×250 μm×0.25 μm) m/z found 355.1 ¹H NMR (400 MHz, Chloroform-d): δ 7.56-7.42 (m, 2H), 7.11 (t, J=8.1 Hz, 2H), 5.70 (q, J=8.1 Hz, 1H), 4.10 (d, J=10.7 Hz, 2H), 3.44-3.32 (m, 2H), 3.33-3.21 (m, 1H), 2.75 (s, 3H), 2.10-1.86 (m, 4H).

### Example 133 and Example 134

### (R)—N-ethyl-N-(2,2,2-trifluoro-1-(4-fluorophenyl)ethyl)pyrimidine-5-sulfonamide (Example 133) and (R)—N-(1-(4-chlorophenyl)-2,2,2-trifluoroethyl)-N-ethylpyrimidine-5-sulfonamide (Example 134)

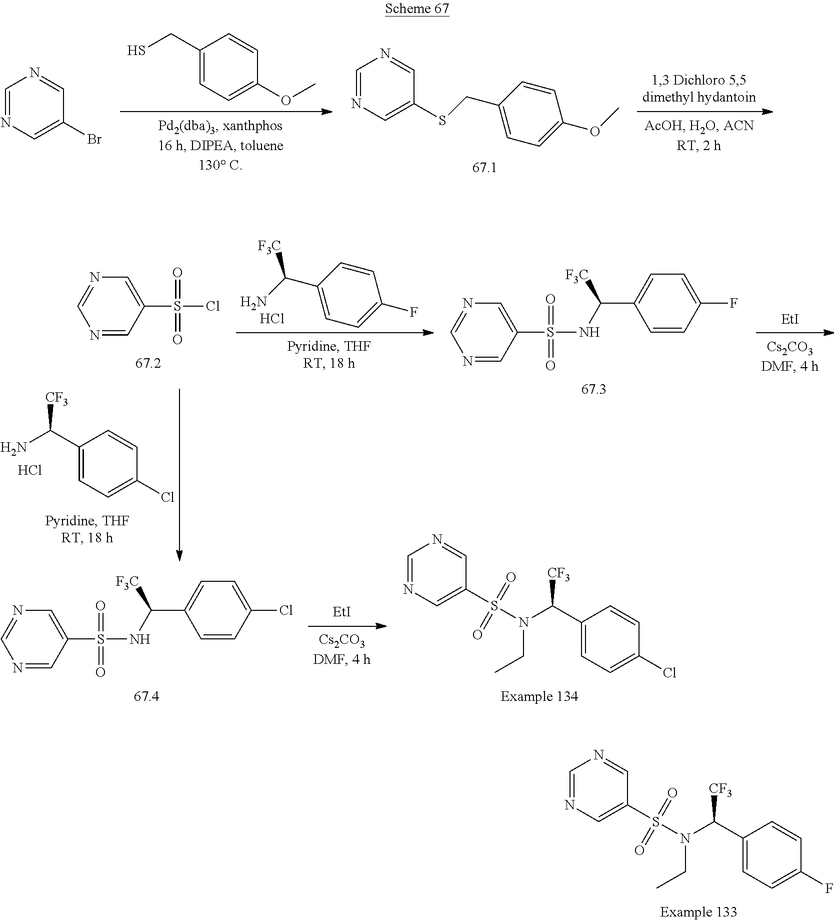

Synthesis of 5-((4-methoxybenzyl)thio)pyrimidine 67.1: Intermediate 67.1 was synthesized from 5-bromopyrimidine following a method analogous to that described in Method I, using (4-methoxyphenyl)methanethiol. The crude residue was directly used for the next step without further purification.

Synthesis of pyrimidine-5-sulfonyl chloride 67.2: Sulfonyl chloride 67.2 was synthesized from intermediate 67.1 following the procedure described in Method T. The crude sulfonyl chloride was used immediately in the following step without further purification.

Synthesis of (R)—N-(2,2,2-trifluoro-1-(4-fluorophenyl)ethyl)pyrimidine-5-sulfonamide 67.3: Intermediate 67.3 was synthesized from sulfonyl chloride 67.2 following the protocol as described in Method O, using (R)-2,2,2-trifluoro-1-(4-fluorophenyl)ethan-1-amine hydrochloride. The compound was purified by column chromatography over silica gel using 3:7 ethyl acetate:hexane. The compound was isolated as a white semi-solid (500 mg, 17.76%). LCMS: m/z found 336.0 [M+H+], rt=2.57 min (Method 9) [Xbridge C18 column (5 μm, 50×4.6 mm)].

Synthesis of (R)—N-ethyl-N-(2,2,2-trifluoro-1-(4-fluorophenyl)ethyl)pyrimidine-5-sulfonamide (Example 133): The ethylation of intermediate 67.3 was preformed following the protocol as described in Method D using Cs₂CO₃ at 60° C. The compound was purified by column chromatography over silica gel using 1:4 ethyl acetate-hexane. Example 133 was isolated as a white solid (60 mg, 13% yield, 96.71% purity). LCMS: m/z found 364.14 [M+H+], rt=3.08 min (Method 2) [Waters Acquity BEH C8 column (1.7 μm, 50×2.1 mm))]; ¹H NMR (400 MHz, Chloroform-d): δ 9.39 (s, 1H), 9.14 (s, 2H), 7.54-7.45 (m, 2H), 7.12 (t, J=8.8 Hz, 2H), 5.81 (q, J=8.4 Hz, 1H), 3.45-3.35 (m, 1H), 3.29-3.14 (m, 1H), 0.95 (t, J=7.1 Hz, 3H).

Synthesis of (R)—N-(1-(4-chlorophenyl)-2,2,2-trifluoroethyl)pyrimidine-5-sulfonamide 67.4: Intermediate 67.4 was synthesized from sulfonyl chloride 67.2 following the protocol as described in Method O using (R)-1-(4-chlorophenyl)-2,2,2-trifluoroethan-1-amine hydrochloride. The compound was directly used for next step without further purification.

Synthesis of (R)—N-(1-(4-chlorophenyl)-2,2,2-trifluoroethyl)-N-ethylpyrimidine-5-sulfonamide (Example 134): The ethylation of intermediate 67.4 was preformed following the protocol as described in Method D using Cs₂CO₃ at 60° C. The compound was purified by column chromatography over silica gel using 1:4 ethyl acetate-hexane. Example 134 was isolated as a colorless oil (15 mg, 42% yield, 99.19% purity). LCMS: m/z found 380.09 [M+H+], rt=3.20 min (Method 2) [Waters Acquity BEH C8 column (1.7 μm, 50×2.1 mm))]; ¹H NMR (400 MHz, DMSO-d6) δ 9.47 (s, 1H), 9.33 (s, 2H), 7.51 (d, J=8.4 Hz, 2H), 7.45 (d, J=8.3 Hz, 2H), 6.13 (q, J=8.2 Hz, 1H), 3.53-3.41 (m, 1H), 3.42-3.32 (m, 1H), 0.99 (t, J=6.9 Hz, 3H).

### Example 135 and Example 136

### N-ethyl-N-(2,2,2-trifluoro-1-(4-fluorophenyl)ethyl)imidazo[1,2-a]pyridine-7-sulfonamide (Example 135) and (R)—N-ethyl-N-(2,2,2-trifluoro-1-(4-fluorophenyl)ethyl)imidazo[1,2-a]pyridine-7-sulfonamide (Example 136)

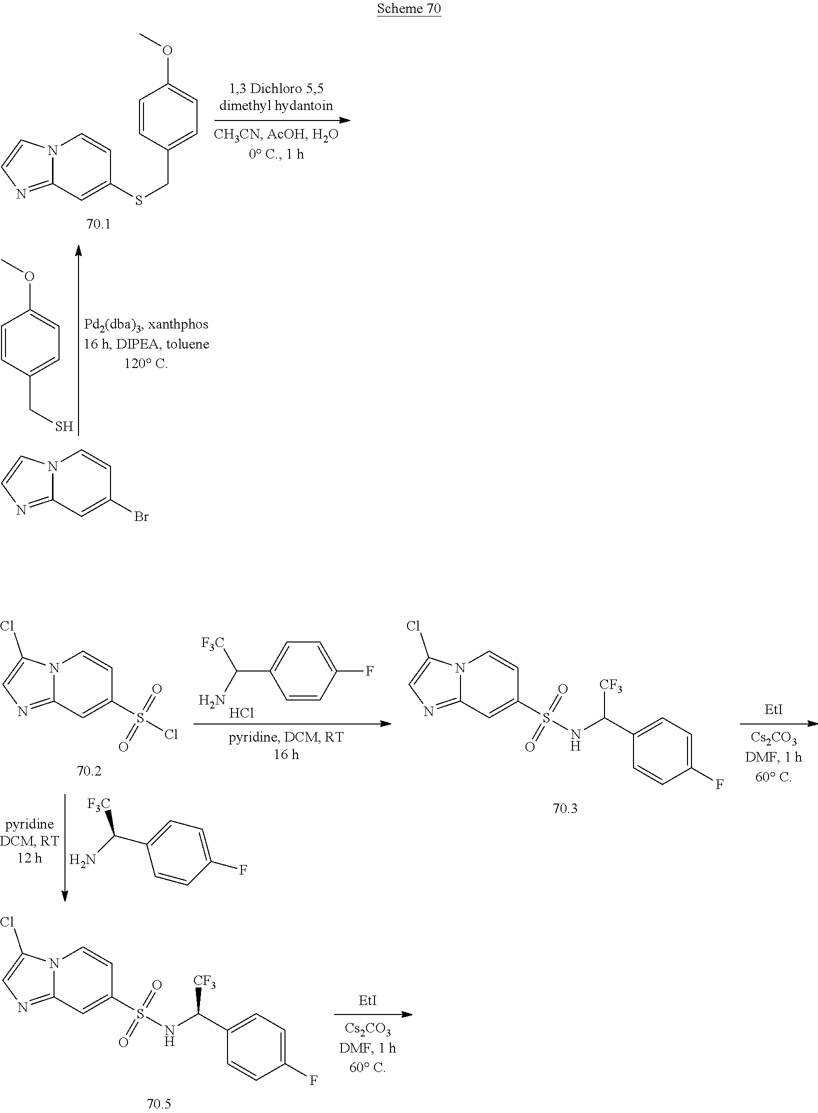

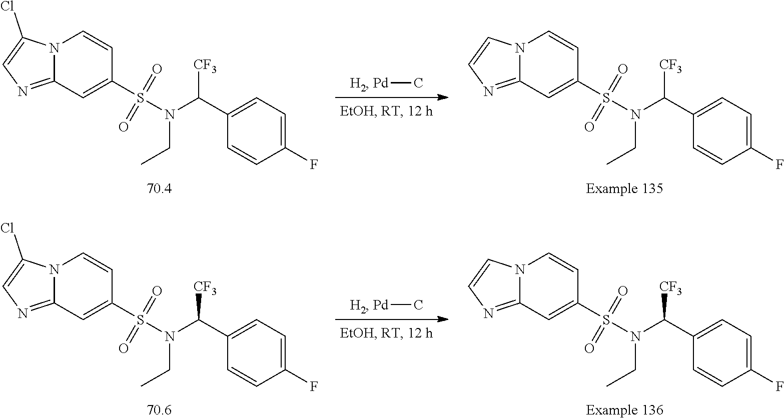

Synthesis of 7-((4-methoxybenzyl)thio)imidazo[1,2-a]pyridine 70.1: Buchwald Coupling; Method AA: 7-bromoimidazo[1,2-a]pyridine (1.0 g, 5.07 mmol) was dissolved in toluene (3 mL) and to this solution (4-methoxyphenyl)methanethiol (0.94 g, 6.09 mmol) was added. The solution was degasified with argon and DIPEA (1.9 mL, 15.22 mmol) was added to it. Xantphos (176 mg, 0.30 mmol) was then added to it under an inert atmosphere, followed by Pd₂(dba)₃ (140 mg, 0.15 mmol). The reaction mixture was stirred at 120° C. for 16 h under argon atmosphere. After completion (monitored by TLC and LCMS) it was diluted with ethyl acetate (10 mL) and filtered through sintered funnel. The filtrate was washed with water (20 mL) and brine (20 mL), dried over anhydrous Na₂SO₄ and the solvent was evaporated under reduced pressure. The crude was triturated with 5% ethyl acetate in hexane to provide the product 70.1 as a yellow gum (1 g, 72% yield).

Synthesis of 3-chloroimidazo[1,2-a]pyridine-7-sulfonyl chloride 70.2: Synthesis of sulfonyl chloride; Method AB: A stirred solution of intermediate 70.1 (300 mg, 1.07 mmol) in MeCN (2 mL) was cooled at 0° C. and to the solution was added a mixture of added AcOH (0.25 mL) and H₂O (0.5 mL). The solution was stirred and 1,3-Dichloro-5,5-dimethyl hydantoin (435 mg, 6.45 mmol) was added portion wise to it. The stirring was continued at the same temperature for 1 h. It was then diluted with ice cold water (5 mL) and extracted with DCM (5 mL). The organic layer was washed with water (5 mL), dried over anhydrous Na₂SO₄. It was then filtered and the organic part was directly used in the forwarding step without evaporation.

Synthesis of 3-chloro-N-(2,2,2-trifluoro-1-(4-fluorophenyl)ethyl)imidazo[1,2-a]pyridine-7-sulfonamide 70.3: Sulfonamidation; Method AC: To the crude sulfonyl chloride 70.2 was added 2,2,2-trifluoro-1-(4-fluorophenyl)ethan-1-amine hydrochloride (165 mg, 0.59 mmol) and pyridine (0.2 mL, 2.98 mmol). The reaction mixture was stirred at room temperature for 16 h. The volatiles were then evaporated under reduced pressure and the crude was purified by column chromatography over silica gel using 3:7 ethyl acetate:hexane to provide compound 70.3 as colorless gum (30 mg, 12%). LCMS: m/z found 407.9 [M+H]⁺, rt=3.13 min (Method 4) [Waters Xbridge C18 column (5 μm, 50×4.6 mm)].

Synthesis of 3-chloro-N-ethyl-N-(2,2,2-trifluoro-1-(4-fluorophenyl)ethyl)imidazo[1,2-a]pyridine-7-sulfonamide 70.4: Alkylation; Method AD: The sulfonamide 70.3 (60 mg, 0.15 mmol) was dissolved in DMF (0.5 mL) in a sealed tube and Cs₂CO₃ (72 mg, 0.22 mmol) was added to it. It was stirred and to it was added ethyl iodide (0.1 mL, 0.29 mmol). The reaction mixture was then stirred at 60° C. for 1 h. The reaction was then quenched with water (5 mL) and extracted with ethyl acetate (2×10 mL). The combined organic layer was washed with water (2×10 mL) and brine (10 mL), dried over anhydrous Na₂SO₄ and concentrated under reduced pressure. The crude was purified by column chromatography over silica gel using 1:4 ethyl acetate-hexane to yield intermediate 70.4 as yellow gum (40 mg, 70% yield). ¹H NMR (400 MHz, DMSO-d₆): δ 8.54 (d, J=7.2 Hz, 1H), 8.26 (s, 1H), 8.00 (s, 1H), 7.51 (t, J=7.8 Hz, 2H), 7.45 (d, J=7.2 Hz, 1H), 7.22 (t, J=8.7 Hz, 2H), 6.15-6.10 (m, 1H), 3.50-3.30 (m, 2H), 0.95 (t, J=6.9 Hz, 3H).

Synthesis of N-ethyl-N-(2,2,2-trifluoro-1-(4-fluorophenyl)ethyl)imidazo[1,2-a]pyridine-7-sulfonamide (Example 135): Hydrogenation; Method AE: To a stirred solution of intermediate 70.4 (50 mg, 0.12 mmol) in ethanol (1 mL) was added Pd on C (10% w/w) (5 mg) and the reaction mixture was stirred in Parr shaker vessel under H₂ atmosphere at 45 psi pressure at room temperature for 12 h. The reaction was then filtered through sintered funnel and the filtrate was concentrated. The crude was purified by column chromatography over silica gel using 1:4 ethyl acetate-hexane to obtain Example 135 as white solid (11 mg, 24% yield, 99.54% purity). LCMS: m/z found 402.1 [M+H]⁺, rt=2.74 min (Method 9) [Waters Xbridge C18 column (3.5 μm, 50×3 mm)]. ¹H NMR (400 MHz, DMSO-d₆): δ 8.73 (d, J=7.0 Hz, 1H), 8.18 (s, 2H), 7.84 (s, 1H), 7.52 (dd, J=5.2, 8.5 Hz, 2H), 7.29 (d, J=6.8 Hz, 1H), 7.22 (t, J=8.6 Hz, 2H), 6.08 (q, J=8.4 Hz, 1H), 3.47-3.33 (m, 2H), 0.96 (t, J=7.0 Hz, 3H).

Synthesis of (R)-3-chloro-N-(2,2,2-trifluoro-1-(4-fluorophenyl)ethyl)imidazo[1,2-a]pyridine-7-sulfonamide 70.5.

Intermediate 70.5 was synthesized from sulfonyl chloride 70.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to obtain compound 70.5 as colorless gum (140 mg, 24%). LCMS: m/z found 407.74 [M+H]⁺, rt=1.88 min (Method 15) [Waters Acquity BEH C18 column (1.7 μm, 50×2.1 mm)].

Synthesis of (R)-3-chloro-N-ethyl-N-(2,2,2-trifluoro-1-(4-fluorophenyl)ethyl)imidazo[1,2-a]pyridine-7-sulfonamide 70.6. The ethylation of intermediate 70.5 was performed following the protocol as described in Method AD. The crude was purified by column chromatography over silica gel using 1:4 ethyl acetate-hexane to obtain compound 70.6 as a colorless gum (50 mg; 87% yield). ¹H NMR (400 MHz, DMSO-d₆): δ 8.28 (s, 1H), 8.20 (d, J=7.2 Hz, 1H), 7.79 (s, 1H), 7.53-7.48 (m, 2H), 7.33 (d, J=7.6 Hz, 1H), 7.11 (t, J=8.4 Hz, 2H), 5.86-5.78 (m, 1H), 3.40-3.35 (m, 1H), 3.22-3.16 (m, 1H), 0.93 (t, J=7.0 Hz, 3H).

Synthesis of (R)—N-ethyl-N-(2,2,2-trifluoro-1-(4-fluorophenyl)ethyl)imidazo[1,2-a]pyridine-7-sulfonamide (Example 136): To a stirred solution of intermediate 70.6 (50 mg, 0.12 mmol) in ethanol (1 mL) 10% Pd on C (2 mg) was added. The reaction mixture was stirred in a Parr shaker vessel under H₂ atmosphere at 45 psi pressure at room temperature for 12 h. The reaction was then filtered through a sintered funnel and the filtrate was concentrated. The crude was purified by column chromatography over silica gel using 1:4 ethyl acetate-hexane to yield Example 136 as an orange solid (28 mg, 46% yield, 98.79% purity). LCMS: m/z found 402.19 [M+H]⁺, rt=3.03 min (Method 9) [Waters Acquity BEH C8 column (1.7 μm, 50×2.1 mm)]. ¹H NMR (400 MHz, Chloroform-d): δ 8.29-8.21 (m, 2H), 7.85 (s, 1H), 7.74 (s, 1H), 7.54-7.46 (m, 2H), 7.17 (d, J=6.7 Hz, 1H), 7.09 (t, J=8.5 Hz, 2H), 5.83 (q, J=8.3 Hz, 1H), 3.44-3.30 (m, 1H), 3.25-3.10 (m, 1H), 0.93 (t, J=7.0 Hz, 3H).

### Example 137

### (R)—N-ethyl-N-(2,2,2-trifluoro-1-(4-fluorophenyl)ethyl)-[1,2,4]triazolo[1,5-a]pyridine-7-sulfonamide (Example 137)

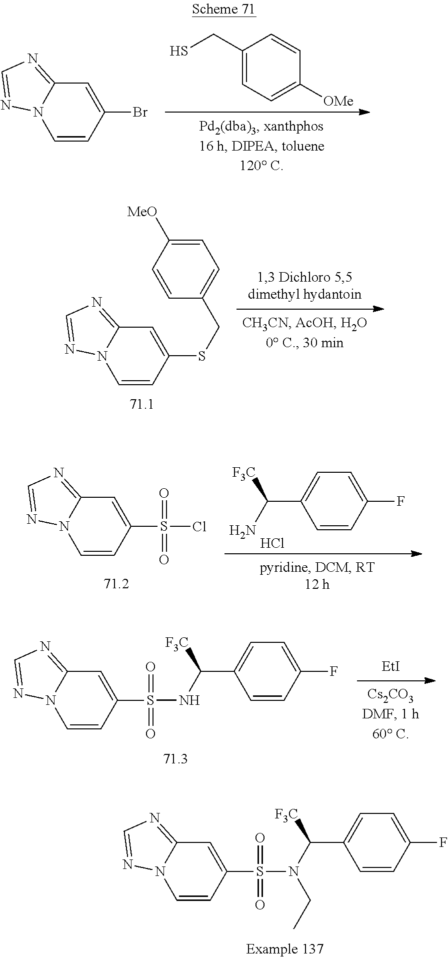

Synthesis of 7-((4-methoxybenzyl)thio)-[1,2,4]triazolo[1,5-a]pyridine 71.1: Intermediate 71.1 was synthesized from 7-bromo-[1,2,4]triazolo[1,5-a]pyridine following a method analogous to that described in Method AA using (4-methoxyphenyl)methanethiol. The reaction mixture was then filtered through a sintered funnel and the residue was collected and concentrated to a residue. The crude residue was purified by column chromatography over silica gel using 1:4 ethyl acetate-hexane to yield the intermediate 71.1 as a yellow sticky solid (500 mg, 71% yield). ¹H NMR (400 MHz, Chloroform-d): δ 8.37 (d, J=6.7 Hz, 1H), 8.24 (s, 1H), 7.49 (s, 1H), 7.31 (d, J=8.5 Hz, 2H), 6.85 (d, J=8.6 Hz, 3H), 4.21 (s, 2H), 3.78 (s, 3H).

Synthesis of [1,2,4]-triazolo[1,5-a]pyridine-7-sulfonyl chloride 71.2: Sulfonyl chloride 71.2 was synthesized from intermediate 71.1 following the procedure described in Method B. The crude sulfonyl chloride was used immediately in the forwarding step without further purification.

Synthesis of (R)—N-(2,2,2-trifluoro-1-(4-fluorophenyl)ethyl)-[1,2,4]triazolo[1,5-a]pyridine-7-sulfonamide 71.3: Intermediate 71.3 was synthesized from sulfonyl chloride 71.2 following the protocol as described in Method AC using (R)-2,2,2-trifluoro-1-(4-fluorophenyl)ethan-1-amine hydrochloride. The compound was purified by column chromatography over silica gel using 3:7 (ethyl acetate:hexane) to provide compound 71.3 as a white, sticky solid (204 mg, 39% yield). ¹H NMR (400 MHz, DMSO-d₆): δ 9.75 (d, J=9.9 Hz, 1H), 9.00 (d, J=7.0 Hz, 1H), 8.67 (s, 1H), 8.06 (s, 1H), 7.50-7.40 (m, 2H), 7.30-7.25 (m, 1H), 6.97 (t, J=8.7 Hz, 2H), 5.55-5.45 (m, 1H).

(R)—N-ethyl-N-(2,2,2-trifluoro-1-(4-fluorophenyl)ethyl)-[1,2,4]triazolo[1,5-a]pyridine-7-sulfonamide (Example 137) The ethylation of intermediate 71.3 was preformed following the protocol as described in Method AD. The crude was purified by column chromatography over silica gel using 1:4 ethyl acetate-hexane to obtain compound Example 137 as colorless oil (55 mg, 25% yield, 96.23% purity). LCMS: m/z found 403.3 [M+H]⁺, rt=3.05 min (Method 2) [Waters Acquity BEH C8 column (1.7 μm, 50×2.1 mm)]. ¹H NMR (400 MHz, DMSO-d6): δ 9.19 (d, J=7.2 Hz, 1H), 8.76 (s, 1H), 8.48 (d, J=1.0 Hz, 1H), 7.62 (dd, J=2.0, 7.2 Hz, 1H), 7.51 (dd, J=5.4, 8.6 Hz, 2H), 7.22 (t, J=8.8 Hz, 2H), 6.16 (q, J=8.6 Hz, 1H), 3.51-3.38 (m, 1H), 3.41-3.27 (m, 1H), 0.98 (t, J=7.0 Hz, 3H).

### Example 138

### (R)—N-ethyl-N-(2,2,2-trifluoro-1-(4-fluorophenyl)ethyl)-[1,2,4]triazolo[4,3-a]pyridine-7-sulfonamide

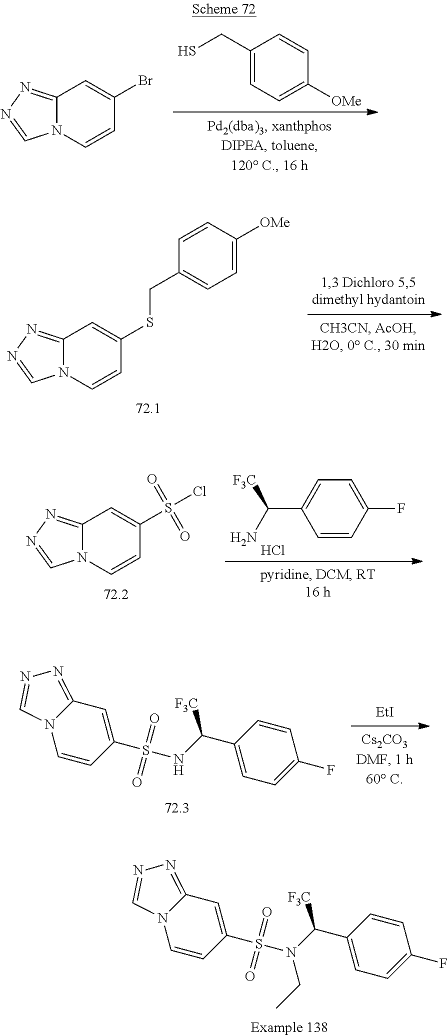

Synthesis of 7-((4-methoxybenzyl)thio)-[1,2,4]triazolo[4,3-a]pyridine 72.1: Intermediate 72.1 was synthesized from of 7-bromo-[1,2,4]triazolo[4,3-a]pyridine following a method analogous to that described in Method AA, using (4-methoxyphenyl)methanethiol. The crude residue was purified by column chromatography over silica gel using 1:4 ethyl acetate-hexane to obtain compound 72.1 as yellow sticky solid (1.2 g, 72% yield). ¹H NMR (400 MHz, DMSO-d6): δ 9.10 (s, 1H), 8.41 (d, J=7.1 Hz, 1H), 7.54 (s, 1H), 7.37 (d, J=8.4 Hz, 2H), 7.0-6.8 (m, 3H), 4.36 (s, 2H), 3.72 (s, 3H).

Synthesis of [1,2,4]triazolo[4,3-a]pyridine-7-sulfonyl chloride 72.2: Sulfonyl chloride 72.2 was synthesized from intermediate 72.1 following the procedure described in Method B. The crude sulfonyl chloride was used immediately in the next step without further purification.

Synthesis of (R)—N-(2,2,2-trifluoro-1-(4-fluorophenyl)ethyl)-[1,2,4]triazolo[4,3-a]pyridine-7-sulfonamide 72.3: Intermediate 72.3 was synthesized from sulfonyl chloride 72.2 following the protocol as described in Method AC using (R)-2,2,2-trifluoro-1-(4-fluorophenyl)ethan-1-amine hydrochloride. The crude was purified by column chromatography over silica gel using 1:4 ethyl acetate-hexane to obtain compound 72.3 as white sticky solid (100 mg, 22% yield). ¹H NMR (400 MHz, DMSO-d6): δ 9.73 (d, J=10.1 Hz, 1H), 9.34 (s, 1H), 8.56 (d, J=6.6 Hz, 1H), 8.04 (s, 1H), 7.94 (s, 1H), 7.50-7.46 (m, 2H), 7.05-6.90 (m, 2H), 5.55-5.40 (m, 1H).

Synthesis of (R)—N-(2,2,2-trifluoro-1-(4-fluorophenyl)ethyl)-[1,2,4]triazolo[4,3-a]pyridine-7-sulfonamide (Example 138): The ethylation of intermediate 72.3 was preformed following the protocol as described in Method AD. The crude was purified by column chromatography over silica gel using 50% ethyl acetate in hexane to provide compound Example 138 was isolated as colorless oil (40 mg, 18% yield, 98.97% purity). LCMS: m/z found 403.18 [M+H]⁺, rt=2.92 min (Method 2) [Waters Acquity BEH C8 column (1.7 μm, 50×2.1 mm)]; ¹H NMR (400 MHz, Chloroform-d): δ 8.96 (s, 1H), 8.43 (s, 1H), 8.24 (d, J=7.2 Hz, 1H), 7.52 (t, J=6.8 Hz, 2H), 7.21 (d, J=8.0 Hz, 1H), 7.12 (t, J=8.4 Hz, 2H), 5.81 (q, J=8.2 Hz, 1H), 3.47-3.36 (m, 1H), 3.28-3.17 (m, 1H), 0.95 (t, J=7.1 Hz, 3H).

### Example 139

### 5-Cyano-N-(2-hydroxyethyl)-N-(2,2,2-trifluoro-1-(4-fluorophenyl)ethyl)pyridine-3-sulfonamide

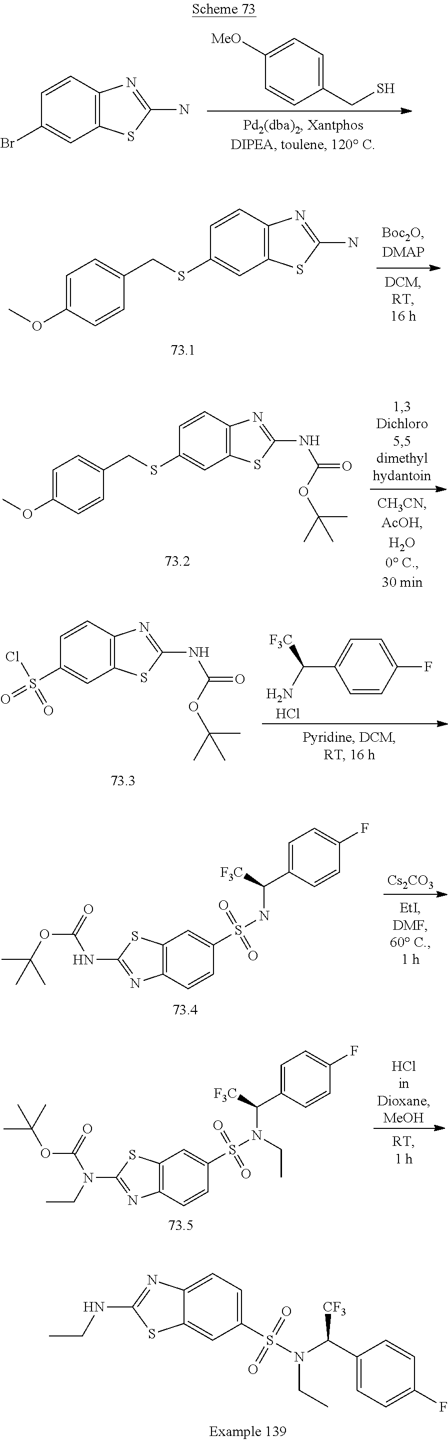

Synthesis of 6-((4-methoxybenzyl)thio)benzo[d]thiazol-2-amine 73.1: Intermediate 73.1 was synthesized from 6-bromobenzo[d]thiazol-2-amine following a method analogous to that described in Method AA using (4-methoxyphenyl)methanethiol. The volatiles were evaporated under reduced pressure and the residue triturated with hexane to get 6-((4-methoxybenzyl)thio)benzo[d]thiazol-2-amine (800 mg crude). LCMS: m/z found 303.0 [M+H]⁺, rt=1.74 min (Method 3) [YMC Triart C18 column (3 μm, 33×2.1 mm)].

Synthesis of tert-butyl (6-((4-methoxybenzyl)thio)benzo[d]thiazol-2-yl)carbamate 4.2: N-Boc protection: Method AF: To the stirred solution of intermediate (800 mg, 2.64 mmol) in DCM (3 ml), Boc₂O (2 mL) was added followed by DMAP (4 mg). The mixture was stirred at room temperature for 16 h. The reaction mixture was then diluted with water (20 mL) and extracted with DCM (2×25 mL), the combined organic layers were washed with water (2×20 mL), dried over anhydrous Na₂SO₄ and the solvent was evaporated under reduced pressure. The crude residue was purified by column chromatography over silica gel using 40% ethyl acetate-hexane to obtain the compound 73.2 which was isolated as a sticky gum (500 mg, 47% yield). ¹H NMR (400 MHz, DMSO-d₆): δ 11.75 (s, 1H), 7.92 (s, 1H), 7.56 (d, J=8.4 Hz, 1H), 7.34 (d, J=8.2 Hz, 1H), 7.23 (d, J=8.04 Hz, 2H), 6.83 (d, J=8.08 Hz, 2H), 4.18 (s, 2H), 3.7 (s, 3H), 1.5 (s, 9H).

Synthesis of tert-butyl (6-(chlorosulfonyl)benzo[d]thiazol-2-yl)carbamate 73.3: Sulfonyl chloride 73.3 was synthesized from Intermediate 73.2 following the procedure described in Method AB. The crude sulfonyl chloride was used immediately in the forwarding step without further purification.

Synthesis of tert-butyl (R)-(6-(N-(2,2,2-trifluoro-1-(4-fluorophenyl)ethyl)sulfamoyl)benzo[d]thiazol-2-yl)carbamate 73.4: Intermediate 733.4 was synthesized from sulfonyl chloride 73.3 following the protocol as described in Method AC using (R)-2,2,2-trifluoro-1-(4-fluorophenyl)ethan-1-amine hydrochloride. The crude residue was purified by column chromatography over silica gel using 35% ethyl acetate in hexane to isolated the sulfonamide 73.3 as yellow sticky gum (150 mg, 12% yield). LCMS: m/z found 506.1 (M+H)+, rt=1.95 min (Method 3) [YMC Triart C18 column (3 μm, 33×2.1 mm)].

Synthesis of tert-butyl (R)-ethyl(6-(N-ethyl-N-(2,2,2-trifluoro-1-(4-fluorophenyl)ethyl)sulfamoyl)benzo[d]thiazol-2-yl)carbamate 73.5: The ethylation of intermediate 73.4 was preformed following the protocol as described in Method AD. The crude residue was purified by column chromatography over silica gel using 5:1 ethyl acetate-hexane to get the desired compound as a colorless sticky solid (80 mg, 27% yield). ¹H NMR (400 MHz, MeOD): δ 8.4 (s, 1H), 7.88-7.81 (m, 2H), 7.44-7.41 (m, 1H), 7.07 (t, J=8.7 Hz, 2H), 5.93-5.86 (m, 2H), 4.31-4.26 (m, 2H), 3.4-3.35 (m, 1H), 3.25-3.21 (m, 1H), 1.62 (s, 9H), 1.32 (t, J=6.9 Hz, 3H), 0.98 (t, J=7.04 Hz, 3H).

Synthesis of (R)—N-ethyl-2-(ethylamino)-N-(2,2,2-trifluoro-1-(4-fluorophenyl)ethyl)benzo[d]thiazole-6-sulfonamide (Example 139): N-Boc-deprotection: Method AG: To the stirred solution of intermediate 73.5 (60 mg, 0.09 mmol) in MeOH (2 ml), 4 M HCl in dioxane (1 mL) was added at room temperature and the mixture was stirred for 2 h. The volatiles were evaporated under reduced pressure and the crude residue was stirred with aqueous K₂CO₃ solution (10 mL). It was then extracted with DCM (2×15 mL), the combined organic layers were washed with water (2×10 mL), dried over anhydrous Na₂SO₄, and the solvent was evaporated under reduced pressure. The crude was purified by Reverse Phase Prep-HPLC and the compound Example 139 was isolated as white solid (15 mg, 39% yield, 99.49% purity) after lyophilization.

Reverse Phase Prep-HPL purification method: Preparative HPLC was done on a WATERS auto purification instrument. Column name: YMC-Actus Triart C18 (250×20 mm, 5μ) operating at ambient temperature and flow rate of 16.0 ml/min. Mobile phase: A=20 mM ammonium carbonate in water, B=Acetonitrile; Gradient Profile: Mobile phase initial composition of 80% A and 20% B, then to 40% A and 60% B in 3 min, then to 22.9% A and 77.1% B in 18 min, then to 5% A and 95% B in 18.5 min, holding this composition up to 21.5 min for column washing, then returned to initial composition in 22 min and held till 25 min. LCMS: m/z found 462.19 [M+H]⁺, rt=3.27 min (Method 2) [Waters Acquity BEH C8 column (1.7 μm, 50×2.1 mm)]; ¹H NMR (400 MHz, DMSO-d₆): δ 8.51 (t, J=5.3 Hz, 1H), 8.24 (d, J=1.9 Hz, 1H), 7.71 (dd, J=2.1, 8.5 Hz, 1H), 7.48-7.38 (m, 3H), 7.20 (t, J=8.8 Hz, 2H), 5.93 (q, J=8.7 Hz, 1H), 3.48-3.36 (m, 2H), 3.36-3.19 (m, 1H), 3.20-3.05 (m, 1H), 1.20 (t, J=7.2 Hz, 3H), 0.91 (t, J=7.0 Hz, 3H).

### Example 140

### (R)—N-ethyl-N-(2,2,2-trifluoro-1-(4-fluorophenyl)ethyl)-5H-pyrrolo[2,3-b]pyrazine-2-sulfonamide

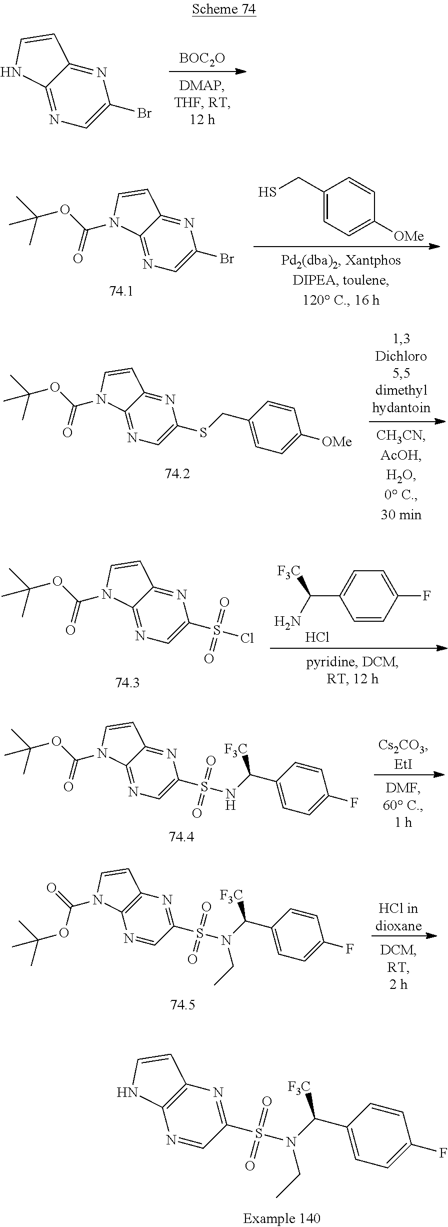

Synthesis of tert-butyl 2-bromo-5H-pyrrolo[2,3-b]pyrazine-5-carboxylate 74.1: Intermediate 74.1 was synthesized from 2-bromo-5H-pyrrolo[2,3-b]pyrazine following a method analogous to that described in Method AF. The crude was purified by column chromatography over silica gel using 20% ethyl acetate-hexane to obtain the compound 74.1 was isolated as yellow gum (2.5 g, 83% yield). ¹H NMR (400 MHz, DMSO-d₆): δ 8.58 (s, 1H), 8.23 (d, J=4.1 Hz, 1H), 6.88 (d, J=4.0 Hz, 1H), 1.61 (s, 1H), 1.66 (s, 9H).

Synthesis of tert-butyl 2-((4-methoxybenzyl)thio)-5H-pyrrolo[2,3-b]pyrazine-5-carboxylate 74.2: Intermediate 74.2 was synthesized from intermediate 74.1 following a method analogous to that described in Method AA using (4-methoxyphenyl)methanethiol. The crude was purified by column chromatography over silica gel using 20% ethyl acetate-hexane to yield compound 74.1 as yellow gum (1.0 g, 80% yield). ¹H NMR (400 MHz, DMSO-d₆): δ 8.33 (s, 1H), 7.81-7.79 (m, 1H), 7.50-7.45 (m, 1H), 7.38-7.30 (m, 2H), 6.91-6.82 (m, 2H), 4.43 (s, 2H), 3.70 (s, 3H), 1.60 (s, 9H).

Synthesis of tert-butyl 2-(chlorosulfonyl)-5H-pyrrolo[2,3-b]pyrazine-5-carboxylate 74.3: Sulfonyl chloride 74.3 was synthesized from Intermediate 74.2 following the procedure described in Method AB. The crude sulfonyl chloride was used immediately in the forwarding step without further purification.

Synthesis of tert-butyl (R)-2-(N-(2,2,2-trifluoro-1-(4-fluorophenyl)ethyl)sulfamoyl)-5H-pyrrolo[2,3-b]pyrazine-5-carboxylate 74.4: Intermediate 74.4 was synthesized from sulfonyl chloride 74.3 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate desired compound 74.4 as yellow sticky gum (70 mg, 11% yield). ¹H NMR (400 MHz, DMSO-d₆): δ 9.70 (d, J=10.2 Hz, 1H), 8.80 (s, 1H), 8.35 (d, J=4.2 Hz, 1H), 7.44-7.40 (m, 2H), 6.97 (d, J=8.8 Hz, 2H), 6.79 (d, J=4.2 Hz, 1H), 5.43-5.37 (m, 1H), 1.62 (s, 9H).

Synthesis of tert-butyl (R)-2-(N-ethyl-N-(2,2,2-trifluoro-1-(4-fluorophenyl)ethyl)sulfamoyl)-5H-pyrrolo[2,3-b]pyrazine-5-carboxylate 74.5: The ethylation of intermediate 74.4 was performed following the protocol as described in Method AD. The crude residue was purified by column chromatography over silica gel using 5:1 ethyl acetate-hexane to obtain compound 74.5 as colorless gum (24 mg, 56% yield). ¹H NMR (400 MHz, CDCl3): δ 9.05 (s, 1H), 8.15 (s, 1H), 7.60-7.50 (m, 2H), 7.07-7.0 (m, 2H), 6.83-6.80 (m, 1H), 5.90-5.80 (m, 1H), 3.50-3.30 (m, 2H), 1.69 (s, 9H), 0.96 (t, J=7.3 Hz, 3H).

Synthesis of (R)—N-ethyl-N-(2,2,2-trifluoro-1-(4-fluorophenyl)ethyl)-5H-pyrrolo[2,3-b]pyrazine-2-sulfonamide (Example 140): N-Boc deprotection of intermediate 74.5 was performed following a method analogous to that described in Method AG. The volatiles were evaporated under reduced pressure and the crude was triturated with 50% diethyl ether-pentane. It was further lyophilized to obtain compound Example 140 as a white sticky solid (15 mg, 96.55% purity, 78% yield). LCMS: m/z found 403.1 [M+H]⁺, rt=2.80 min (Method 9) [Waters Xbridge C18 column (3.5 μm, 50×3 mm)]; ¹H NMR (400 MHz, DMSO-d₆): δ 12.69 (s, 1H), 8.81 (s, 1H), 8.20 (t, J=3.1 Hz, 1H), 7.53 (dd, J=5.3, 8.6 Hz, 2H), 7.18 (t, J=8.8 Hz, 2H), 6.83 (dd, J=1.7, 3.7 Hz, 1H), 5.97 (q, J=8.8 Hz, 1H), 3.44-3.32 (m, 1H), 1.30-1.17 (m, 1H), 0.91 (t, J=7.0 Hz, 3H).

### Example 141

### (R)-3-cyano-N-ethyl-N-(2,2,2-trifluoro-1-(4-fluorophenyl)ethyl)pyrazolo[1,5-a]pyrimidine-6-sulfonamide

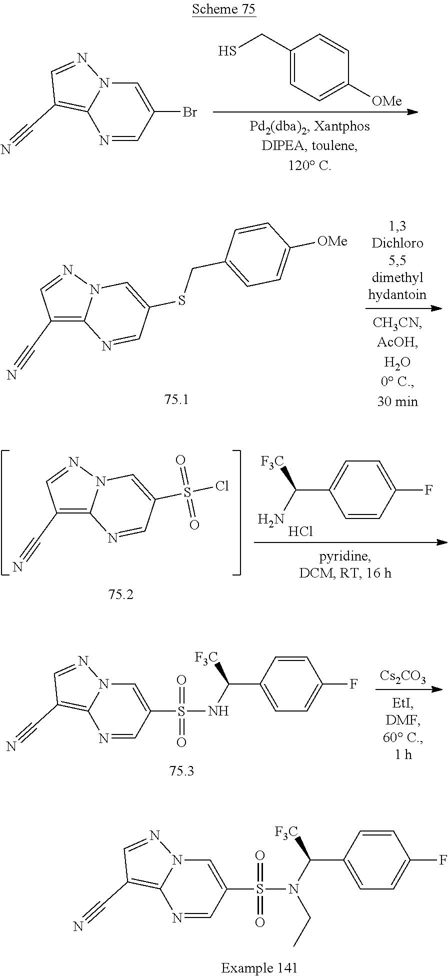

Synthesis of 6-((4-methoxybenzyl)thio)pyrazolo[1,5-a]pyrimidine-3-carbonitrile 75.1: Intermediate 75.1 was synthesized from 6-bromopyrazolo[1,5-a]pyrimidine-3-carbonitrile following a method analogous to that described in Method AA using (4-methoxyphenyl)methanethiol. The crude was purified by column chromatography over silica gel using 20% ethyl acetate-hexane to isolate the compound 75.1 as yellow gum (1.3 g, 90% yield). ¹H NMR (400 MHz, DMSO-d₆): δ 9.36 (d, J=2.0 Hz, 1H), 8.80 (s, 1H), 8.71 (d, J=2.1 Hz, 1H), 7.22 (d, J=8.5 Hz, 2H), 6.90-6.83 (m, 2H), 4.27 (s, 2H), 3.70 (s, 3H).

Synthesis of 3-cyanopyrazolo[1,5-a]pyrimidine-6-sulfonyl chloride 75.2: Sulfonyl chloride 75.2 was synthesized from Intermediate 75.1 following the procedure described in Method AB. The crude sulfonyl chloride was used immediately in the forwarding step without further purification.

Synthesis of (R)-3-cyano-N-(2,2,2-trifluoro-1-(4-fluorophenyl)ethyl)pyrazolo[1,5-a]pyrimidine-6-sulfonamide 75.3: Intermediate 75.3 was synthesized from sulfonyl chloride 75.2 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate the compound as a yellow sticky gum (170 mg, 26% yield). ¹H NMR (400 MHz, DMSO-d₆): δ 10-9.9 (m, 1H), 9.53 (s, 1H), 9.0 (s, 1H), 8.83 (d, J=1.8 Hz, 1H), 7.50-7.40 (m, 2H), 7.02 (d, J=9.2 Hz, 2H), 5.55-5.45 (m, 1H).

Synthesis of (R)-3-cyano-N-ethyl-N-(2,2,2-trifluoro-1-(4-fluorophenyl)ethyl)pyrazolo[1,5-a]pyrimidine-6-sulfonamide (Example 141): The ethylation of intermediate 75.3 was preformed following the protocol as described in Method AD. The crude was purified by Reverse Phase Prep-HPLC and the compound Example 141 was isolated as white solid (10 mg, 99.96% purity, 5% yield) after lyophilization. RP method: Preparative HPLC was done on WATERS auto purification instrument. Column name: xbridge C18 (250×19 mm, 5μ) operating at ambient temperature and flow rate of 16.0 ml/min. Mobile phase: A=20 mM ammonium bicarbonate in water, B=Methanol; Gradient Profile: Mobile phase initial composition of 80% A and 20% B, then to 40% A and 60% B in 3 min., then to 20% A and 80% B in 18 min., then to 5% A and 95% B in 18.5 min., held this composition up to 21.5 min. for column washing, then returned to initial composition in 22 min. and held till 25 mins. LCMS: m/z found 426.07 [M−H], rt=3.24 min (Method 2) [Waters Acquity BEH C8 column (1.7 μm, 50×2.1 mm)]; ¹H NMR (400 MHz, DMSO-d₆): δ 9.98 (s, 1H), 9.20 (s, 1H), 9.06 (s, 1H), 7.52 (t, J=5.2 Hz, 2H), 7.22 (t, J=8.6 Hz, 2H), 6.16 (q, J=8.7 Hz, 1H), 3.55-3.34 (m, 2H), 1.03 (t, J=6.9 Hz, 3H).

### Example 142 and Example 143

### (R)—N-ethyl-N-(2,2,2-trifluoro-1-(4-fluorophenyl)ethyl)imidazo[1,2-a]pyridine-2-sulfonamide (Example 142) and (R)-3-chloro-N-ethyl-N-(2,2,2-trifluoro-1-(4-fluorophenyl)ethyl)imidazo[1,2-a]pyridine-2-sulfonamide (Example 143)

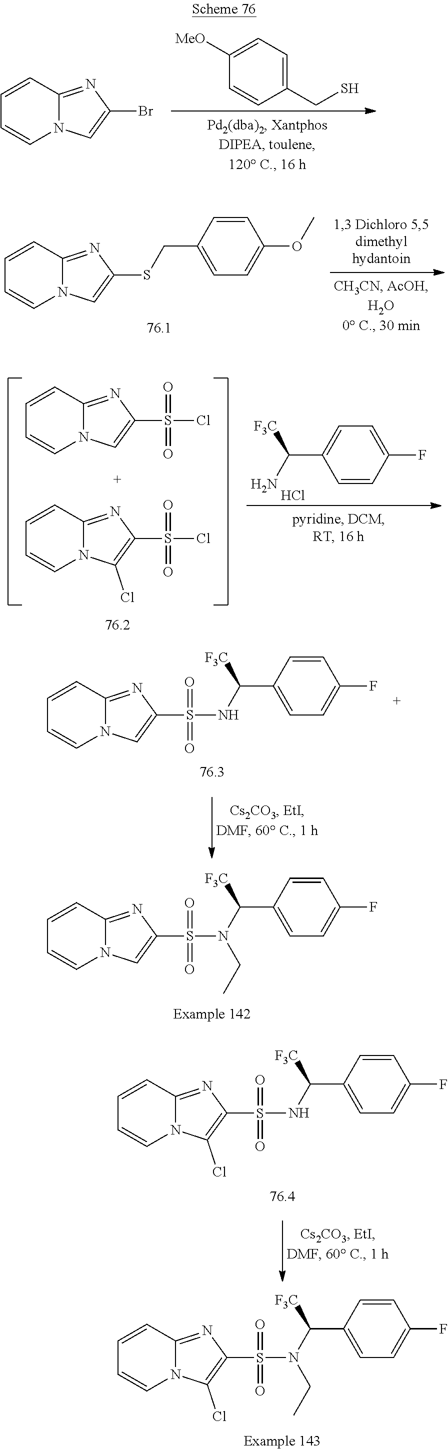

Synthesis of 2-((4methoxybenzyl)thio)imidazo[1,2-a]pyridine 76.1: Intermediate 76.1 was synthesized from 2-bromoimidazo[1,2-a]pyridine following a method analogous to that described in Method A using (4-methoxyphenyl)methanethiol. The crude was purified by column chromatography over silica gel using 50% ethyl acetate-hexane to isolate the compound 76.1 as a sticky gum (750 mg, 55% yield). ¹H NMR (400 MHz, DMSO-d₆): δ 8.47-8.41 (m, 1H), 7.48-7.42 (m, 2H), 7.21 (s, 1H), 6.48-6.42 (m, 5H), 3.99 (s, 2H), 3.81 (s, 3H).

Synthesis of imidazo[1,2-a]pyridine-2-sulfonyl chloride and 3-chloroimidazo[1,2-a]pyridine-2-sulfonyl chloride 76.2: Sulfonyl chlorides 76.2 was synthesized from intermediate 76.1 following the procedure described in Method AB. The crude mixture of sulfonyl chlorides was used immediately in the forwarding step without further purification.

Synthesis of (R)—N-(2,2,2-trifluoro-1-(4-fluorophenyl)ethyl)imidazo[1,2-a]pyridine-2-sulfonamide 76.3 and (R)-3-chloro-N-(2,2,2-trifluoro-1-(4-fluorophenyl)ethyl)imidazo[1,2-a]pyridine-2-sulfonamide 76.4: Intermediate 76.3 was synthesized from sulfonyl chloride 76.2 following the protocol as described in Method AC using (R)-2,2,2-trifluoro-1-(4-fluorophenyl)ethan-1-amine hydrochloride. The crude residue was purified by column chromatography over silica gel using 3:7 ethyl acetate:hexane to get the compound 76.3 as a yellow gum (90 mg, 45% yield). ¹H NMR (400 MHz, DMSO-d₆): δ 9.37 (d, J=8.2 Hz, 1H), 8.47 (d, J=6.72 Hz, 1H), 8.24 (s, 1H), 7.42-7.31 (m, 3H), 6.98-6.91 (m, 3H), 5.21 (s, 1H). Compound 76.4 was isolated in a mixture containing both compound 76.3 and 76.4.

Synthesis of (R)—N-ethyl-N-(2,2,2-trifluoro-1-(4-fluorophenyl)ethyl)imidazo[1,2-a]pyridine-2-sulfonamide (Example 142): The ethylation of intermediate 76.3 was preformed following the protocol as described in Method AD at 60° C. The crude was purified by column chromatography over silica gel using 1:4 ethyl acetate-hexane to obtain the compound Example 142 as white solid (60 mg, 45% yield, 97.15% purity). LCMS: m/z found 402.18 [M+H]⁺, rt=3.10 min (Method 2) [Waters Acquity BEH C8 column (1.7 μm, 50×2.1 mm)]; ¹H NMR (400 MHz, Chloroform-d): δ 8.12 (d, J=6.6 Hz, 1H), 8.02 (s, 1H), 7.67 (d, J=9.0 Hz, 1H), 7.55 (dd, J=5.1, 8.5 Hz, 2H), 7.33 (t, J=7.8 Hz, 1H), 7.00 (t, J=8.4 Hz, 2H), 6.94 (t, J=6.6 Hz, 1H), 5.88 (q, J=8.5 Hz, 1H), 3.42-3.30 (m, 2H), 1.05 (t, J=6.8 Hz, 3H).

Synthesis of (R)-3-chloro-N-ethyl-N-(2,2,2-trifluoro-1-(4-fluorophenyl)ethyl)imidazo[1,2-a]pyridine-2-sulfonamide (Example 143): The ethylation of intermediate 76.4 was preformed following the protocol as described in Method AD. The crude residue was purified by column chromatography over silica gel using 1:4 ethyl acetate-hexane to get the compound Example 143 as a white solid (25 mg, 39% yield, 99.84% purity). LCMS: m/z found 436.17 [M+H]⁺, rt=3.17 min(Method 2) [Waters Acquity BEH C8 column (1.7 μm, 50×2.1 mm)]; ¹H NMR (400 MHz, CDCl₃): δ 8.12 (d, J=6.8 Hz, 1H), 7.66 (d, J=9.1 Hz, 1H), 7.54 (t, J=5.3 Hz, 2H), 7.39 (t, J=8.1 Hz, 1H), 7.07 (t, J=6.7 Hz, 1H), 7.00 (t, J=8.4 Hz, 2H), 5.89 (q, J=8.3 Hz, 1H), 3.54-3.33 (m, 2H), 1.06 (t, J=7.0 Hz, 3H).

### Example 144

### (R)—N-ethyl-N-(2,2,2-trifluoro-1-(4-fluorophenyl)ethyl)-[1,2,4]triazolo[1,5-a]pyrazine-2-sulfonamide

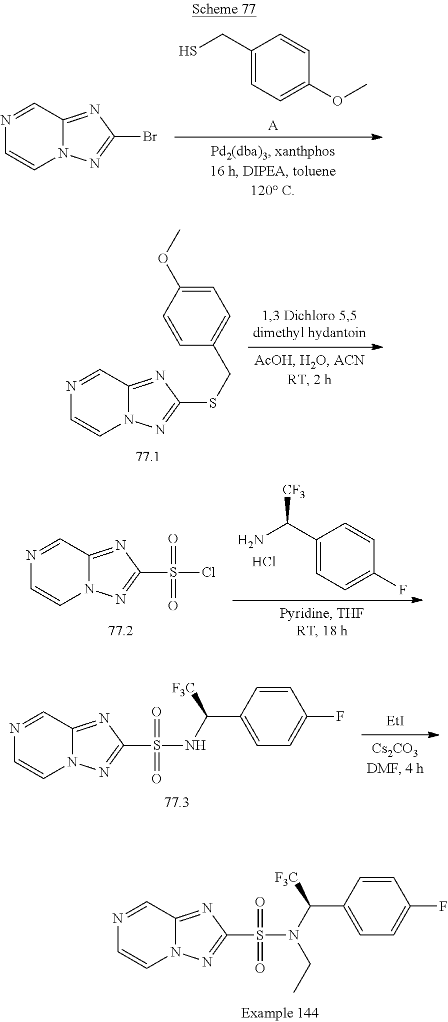

Synthesis of 2-((4-methoxybenzyl)thio)-[1,2,4]triazolo[1,5-a]pyrazine 77.1: Intermediate 77.1 was synthesized from 2-bromo-[1,2,4]triazolo[1,5-a]pyrazine following a method analogous to that described in Method AA using (4-methoxyphenyl)methanethiol. The reaction was filtered through a sintered funnel and the filtrate was collected and concentrated to a residue. The crude residue was purified by column chromatography over silica gel using 3:7 ethyl acetate:hexane to yield the compound 77.1 as a yellow gum (750 mg, 65% yield). ¹H NMR (400 MHz, DMSO-d₆): δ 9.28 (d, 1H), 9.02 (d, J=3.1 Hz, 1H), 8.22 (d, J=4.3 Hz, 1H), 7.40 (d, J=8.4 Hz, 2H), 6.87 (d, J=8.4 Hz, 2H), 4.50 (s, 2H), 3.71 (s, 3H).

Synthesis of [1,2,4]triazolo[1,5-a]pyrazine-2-sulfonyl chloride 77.2: Sulfonyl chloride 77.2 was synthesized from intermediate 77.1 following the procedure described in Method AB. The crude sulfonyl chloride was used immediately in the next step without further purification.

Synthesis of (R)—N-(2,2,2-trifluoro-1-(4-fluorophenyl)ethyl)-[1,2,4]triazolo[1,5-a]pyrazine-2-sulfonamide 77.3: intermediate 77.3 was synthesized from [1,2,4]triazolo[1,5-a]pyrazine-2-sulfonyl chloride 77.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to isolate compound 77.3 as white sticky solid (80 mg, 31% yield). ¹H NMR (400 MHz, DMSO-d₆): b 10.23 (d, J=9.6 Hz, 1H), 9.40 (d, J=0.8 Hz, 1H), 9.10-9.05 (m, 1H), 8.40 (d, J=4.5 Hz, 1H), 7.50-7.40 (m, 2H), 7.00 (t, J=8.8 Hz, 2H), 5.39 (t, J=8.8 Hz, 1H).

Synthesis of (R)—N-ethyl-N-(2,2,2-trifluoro-1-(4-fluorophenyl)ethyl)-[1,2,4]triazolo[1,5-a]pyrazine-2-sulfonamide (Example 144): The ethylation of intermediate 8.3 was preformed following the protocol as described in Method D. The crude was purified by column chromatography over silica gel using 1:4 ethyl acetate-hexane to get Example 144 as a yellow solid (20 mg, 26% yield, 97.35% purity). LCMS: m/z found 404.17 [M+H]⁺, rt=3.12 min (Method 2) Waters Acquity BEH C8 column (1.7 μm, 50×2.1 mm); ¹H NMR (400 MHz, DMSO-d₆): δ 9.57 (s, 1H), 9.21 (d, J=4.1 Hz, 1H), 8.48 (d, J=4.4 Hz, 1H), 7.51 (t, J=5.4 Hz, 2H), 7.19 (t, J=8.6 Hz, 2H), 5.96 (q, J=8.7 Hz, 1H), 3.56-3.38 (m, 2H), 1.01 (t, J=6.9 Hz, 3H).

### Example 145

### (R)—N-ethyl-N-(2,2,2-trifluoro-1-(4-fluorophenyl)ethyl)-[1,2,3]triazolo[1,5-a]pyridine-5-sulfonamide

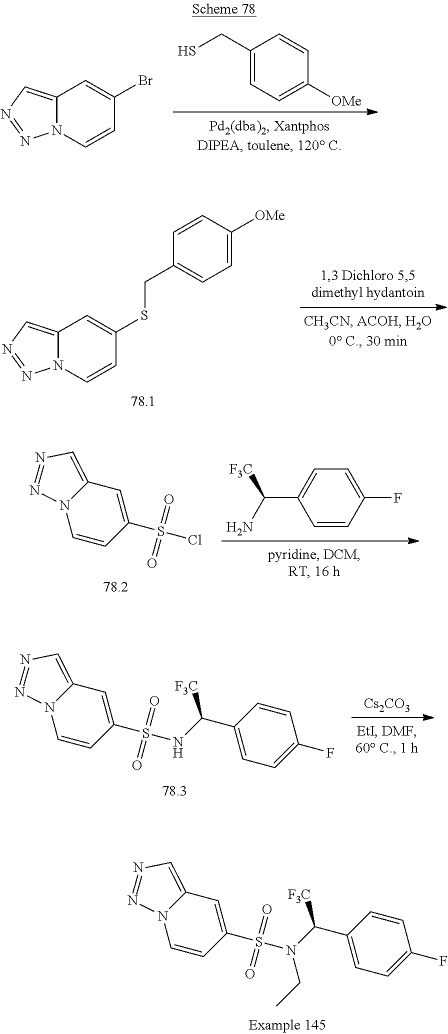

Synthesis of 4-methoxy-5-((4-methoxybenzyl)thio)nicotinonitrile 78.1: Intermediate 78.1 was synthesized following the Buchwald protocol as described in method Method AA using (4-methoxyphenyl)methanethiol. The crude was purified by column chromatography over silica gel using 50% ethyl acetate in hexane to obtain desired compound 78.1 (72% yield) as yellow gum. LCMS: m/z found 272.34 [M+H+], rt=2.72 min (Method 2) Waters Acquity BEH C8 column (1.7 μm, 50×2.1 mm).

Synthesis of [1,2,3]triazolo[1,5-a]pyridine-5-sulfonyl chloride 78.2: Intermediate 78.2 was synthesized following the protocol as described in Method AB. The crude sulfonyl chloride 78.2 was used for next step sulfonamidation reaction without any purification.

Synthesis of (R)—N-(2,2,2-trifluoro-1-(4-fluorophenyl)ethyl)-[1,2,3]triazolo[1,5-a]pyridine-5-sulfonamide 78.3: Intermediate 78.3 was synthesized following the protocol as described in Method AC using (R)-2,2,2-trifluoro-1-(4-fluorophenyl)ethan-1-amine hydrochloride. The crude was purified by column chromatography over silica gel using 30% ethyl acetate in hexane to obtain desired compound 78.3 (300 mg, 52% yield) as a colourless sticky gum. LCMS: m/z found 375.0[M+H+], rt=2.79 min (Method 9) [Waters Xbridge C18 column (3.5 μm, 50×3 mm)].

Synthesis of (R)—N-ethyl-N-(2,2,2-trifluoro-1-(4-fluorophenyl)ethyl)-[1,2,3]triazolo[1,5-a]pyridine-5-sulfonamide (Example 145): The ethylation of intermediate 78.3 was performed following the protocol as described in Method AD. The crude was purified by column chromatography over silica gel using 30% ethyl acetate in hexane to obtain compound Example 145 (75 mg, 62% yield, 99.63% Purity) as white solid. LCMS: m/z found 403.18 [M+H]⁺, rt=3.09 min (Method 2) [Waters Acquity BEH C8 column (1.7 μm, 50×2.1 mm)]; ¹H NMR (400 MHz, Chloroform-d): δ 8.82 (d, J=7.3 Hz, 1H), 8.36 (s, 1H), 8.30 (s, 1H), 7.56-7.47 (m, 2H), 7.30 (d, J=7.2 Hz, 1H), 7.12 (t, J=8.4 Hz, 2H), 5.82 (q, J=8.0 Hz, 1H), 3.48-3.33 (m, 1H), 3.28-3.14 (m, 1H), 0.94 (t, J=7.0 Hz, 3H).

### Example 146

### (R)—N-ethyl-N-(2,2,2-trifluoro-1-(4-fluorophenyl)ethyl)imidazo[1,2-a]pyridine-6-sulfonamide

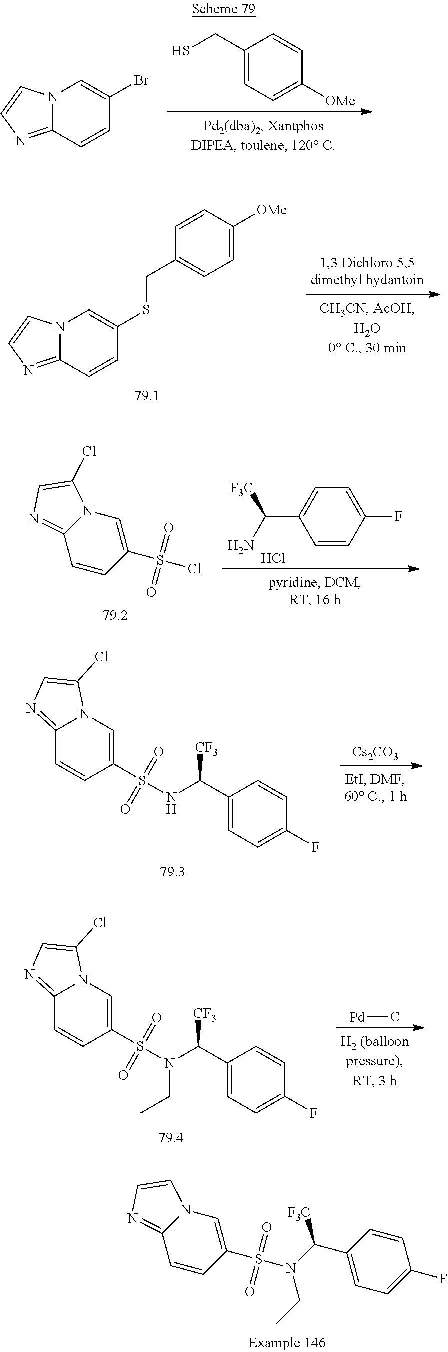

Synthesis of 6-((4-methoxybenzyl)thio)imidazo[1,2-a]pyridine 79.1: Intermediate 79.1 was synthesized from 6-bromoimidazo[1,2-a]pyridine following a method analogous to that described in Method AA using (4-methoxyphenyl)methanethiol. The crude was purified by column chromatography over silica gel using 20% ethyl acetate-hexane to get the compound 79.1 as yellow gum. (1.0 g, 78% yield). LCMS: m/z found 271.0 [M+H]⁺, rt=1.53 min (Method 3) [YMC Triart C18 column (3 μm, 33×2.1 mm)].

Synthesis of 3-chloroimidazo[1,2-a]pyridine-6-sulfonyl chloride 79.2: Sulfonyl chloride 79.2 was synthesized from intermediate 79.1 following the procedure described in Method AB. The crude sulfonyl chloride was used immediately in the forwarding step without further purification.

Synthesis of (R)-3-chloro-N-(2,2,2-trifluoro-1-(4-fluorophenyl)ethyl)imidazo[1,2-a]pyridine-6-sulfonamide 79.3: Intermediate 79.3 was synthesized from sulfonyl chloride 79.2 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate the desired compound as yellow gum (250 mg, 16% yield). ¹H NMR (400 MHz, DMSO-d₆): δ 9.59 (d, J=10.3 Hz, 1H), 8.45 (s, 1H), 7.61 (d, J=9.5 Hz, 1H), 7.42 (t, J=7.5 Hz, 2H), 7.31 (t, J=8.6 Hz, 2H), 6.93 (t, J=8.6 Hz, 2H), 5.60-5.45 (m, 1H).

Synthesis of (R)-3-chloro-N-ethyl-N-(2,2,2-trifluoro-1-(4-fluorophenyl)ethyl)imidazo[1,2-a]pyridine-6-sulfonamide 79.4: The ethylation of intermediate 79.3 was performed following the protocol as described in Method AD. The crude was purified by column chromatography over silica gel using 5:1 ethyl acetate-hexane to yield the compound as a colorless gum (100 mg, 25% yield). ¹H NMR (400 MHz, DMSO-d₆): δ 8.76 (s, 1H), 7.79 (d, J=9.5 Hz, 1H), 7.62 (dd, J=9.5 Hz, 1.8 Hz, 1H), 7.51-7.46 (m, 2H), 7.20 (t, J=8.8 Hz, 2H), 6.20-6.0 (m, 1H), 3.60-3.30 (m, 2H), 1.02 (t, J=6.9 Hz, 3H).

Synthesis of (R)—N-ethyl-N-(2,2,2-trifluoro-1-(4-fluorophenyl)ethyl)imidazo[1,2-a]pyridine-6-sulfonamide (Example 146): A solution of intermediate 79.4 (110 mg, 0.25 mmol) in ethanol (10 mL) was degassed with argon and to it was added Pd on C (10% w/w) (20 mg). The RM was then stirred under H₂ atmosphere (balloon pressure) at room temperature for 3 h. It was then filtered through a sintered funnel and the filtrate was concentrated. The crude was purified by Reverse Phase Prep-HPLC and the compound Example 146 was isolated as white solid (25 mg, 99.66% purity, 25% yield) after lyophilization. RP method: Preparative HPLC was done on a WATERS BGM 2545 equipped with WATERS PDA detector 2998 set to multiple-wavelength UV (200-400 nm) detection. Column name: YMC Actus Triart C18 (250×20 mm, 5μ) operating at ambient temperature and flow rate of 16 mL/min. Mobile phase: A=20 mM ammonium bicarbonate in water, B=Acetonitrile. Gradient profile: Mobile phase initial composition of 60% A and 40% B, then to 10% A and 90% B in 20 min, then to 0% A and 100% B in 22 min, held in this composition up to 24 min for column washing, then returned to initial composition in 24.5 min an held till 28 min. LCMS: m/z found 402.1 [M+H]⁺, rt=2.73 min (Method 9) [Waters Xbridge C18 column (3.5 μm, 50×3 mm)]; ¹H NMR (400 MHz, DMSO-d₆): δ 9.34 (d, J=1.9 Hz, 1H), 8.11 (s, 1H), 7.74 (s, 1H), 7.70 (d, J=9.6 Hz, 1H), 7.57-7.45 (m, 3H), 7.22 (t, J=8.8 Hz, 2H), 6.01 (q, J=8.7 Hz, 1H), 3.45-3.31 (m, 1H), 3.32-3.19 (m, 1H), 0.98 (t, J=7.0 Hz, 3H). ¹H NMR (400 MHz, DMSO-d₆): δ 9.34 (d, J=1.9 Hz, 1H), 8.11 (s, 1H), 7.74 (s, 1H), 7.70 (d, J=9.6 Hz, 1H), 7.57-7.45 (m, 3H), 7.22 (t, J=8.8 Hz, 2H), 6.01 (q, J=8.7 Hz, 1H), 3.45-3.31 (m, 1H), 3.32-3.19 (m, 1H), 0.98 (t, J=7.0 Hz, 3H).

### Example 147

### (R)—N-ethyl-N-(2,2,2-trifluoro-1-(4-fluorophenyl)ethyl)-[1,2,5]thiadiazolo[3,4-b]pyridine-6-sulfonamide

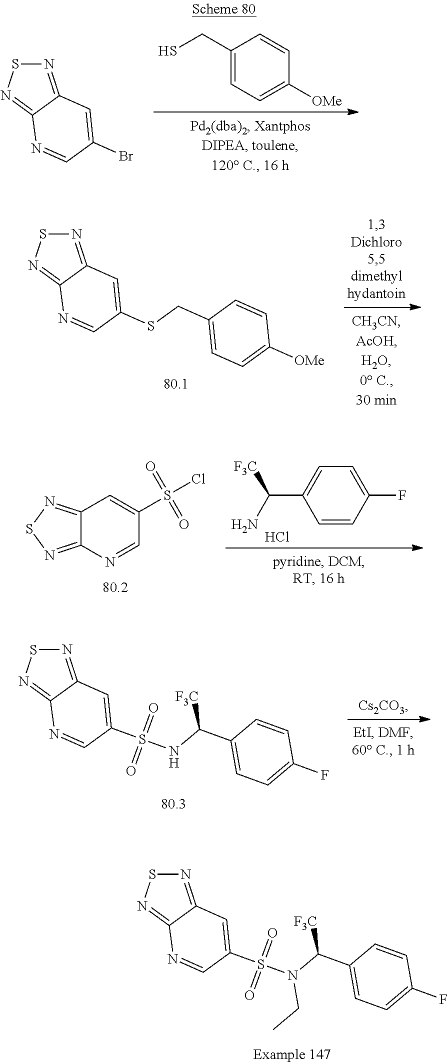

Synthesis of 6-((4-methoxybenzyl)thio)-[1,2,5]thiadiazolo[3,4-b]pyridine 80.1: Intermediate 80.1 was synthesized from 6-bromo-[1,2,5]thiadiazolo[3,4-b]pyridine following a method analogous to that described in Method AA using (4-methoxyphenyl)methanethiol. The crude was purified by column chromatography over silica gel using 20% ethyl acetate-hexane to isolate the compound 80.1 as a yellow gum. (600 mg, 87% yield). ¹H NMR (400 MHz, DMSO-d₆): δ 8.98 (d, J=2.3 Hz, 1H), 8.36 (d, J=2.0 Hz, 1H), 7.39 (d, J=8.6 Hz, 2H), 6.91-6.80 (m, 2H), 4.45 (s, 2H), 3.71 (s, 3H).

Synthesis of [1,2,5]thiadiazolo[3,4-b]pyridine-6-sulfonyl chloride 80.2: Sulfonyl chloride 80.2 was synthesized from Intermediate 80.1 following the procedure described in Method AB. The crude sulfonyl chloride was used immediately in the forwarding step without further purification.

Synthesis of (R)—N-(2,2,2-trifluoro-1-(4-fluorophenyl)ethyl)-[1,2,5]thiadiazolo[3,4-b]pyridine-6-sulfonamide 80.3: Intermediate 80.3 was synthesized from sulfonyl chloride 80.2 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to provide 80.3 as a yellow sticky gum (100 mg, 13% yield). ¹H NMR (400 MHz, DMSO-d₆): δ 9.90 (d, Hz, 1H J=9.9), 9.18 (s, 1H), 8.76 (s, 1H), 7.42 (t, J=8.1 Hz, 2H), 6.94 (t, J=8.6 Hz, 2H), 5.60-5.50 (m, 1H).

Synthesis of (R)—N-ethyl-N-(2,2,2-trifluoro-1-(4-fluorophenyl)ethyl)-[1,2,5]thiadiazolo[3,4-b]pyridine-6-sulfonamide Example 147: The ethylation of intermediate 80.3 was preformed following the protocol as described in Method AD. The crude was purified by column chromatography over silica gel using 5:1 ethyl acetate-hexane to obtain the product Example 147 as a colorless gum (15 mg, 16% yield). LCMS: m/z found 421.15 [M+H]⁺, rt=3.18 min (Method 2) [Waters Acquity BEH C8 column (1.7 μm, 50×2.1 mm)]; ¹H NMR (400 MHz, DMSO-d₆): δ 9.51 (d, J=2.3 Hz, 1H), 9.21 (d, J=2.3 Hz, 1H), 7.51 (dd, J=5.3, 8.7 Hz, 2H), 7.22 (t, J=8.8 Hz, 2H), 6.22 (q, J=8.7 Hz, 1H), 3.58-3.35 (m, 2H), 1.00 (t, J=7.0 Hz, 3H).

### Example 148

### (R)—N-ethyl-N-(2,2,2-trifluoro-1-(4-fluorophenyl)ethyl)pyrazolo[1,5-a]pyrimidine-2-sulfonamide

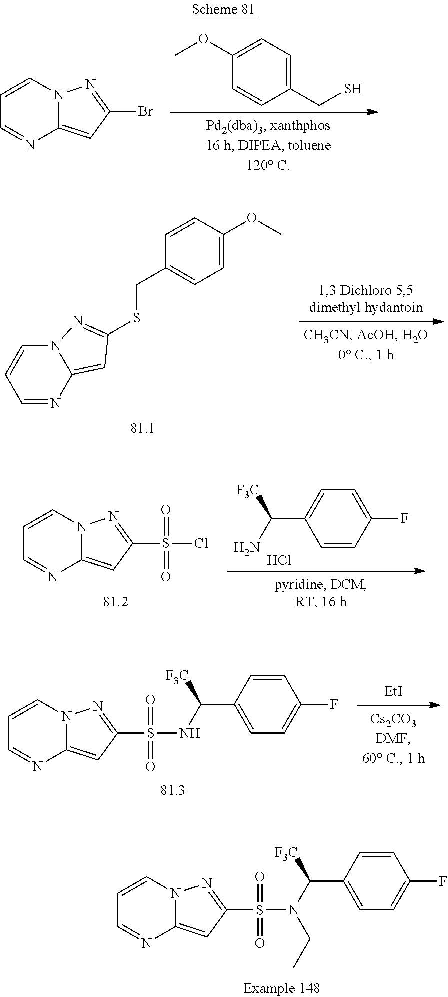

Synthesis of 2-((4-methoxybenzyl)thio)pyrazolo[1,5-a]pyrimidine 81.1: Intermediate 81.1 was synthesized from 2-bromopyrazolo[1,5-a]pyrimidine following a method analogous to that described in Method AA using (4-methoxyphenyl)methanethiol. The reaction was then filtered through a sintered funnel and the filtrate was evaporated under reduced pressure. The crude was purified by column chromatography over silica gel using 5:1 ethyl acetate-hexane to obtain 81.1 as a yellow gum (550 mg, 71% yield). ¹H NMR (400 MHz, DMSO-d₆): δ 9.01 (d, J=6.7 Hz, 1H), 8.50-8.46 (m, 1H), 7.35 (d, J=8.5 Hz, 2H), 7.0-6.90 (m, 1H), 6.86 (t, J=8.6 Hz, 2H), 6.72 (s, 1H), 4.35 (s, 2H), 3.71 (s, 3H).

Synthesis of pyrazolo[1,5-a]pyrimidine-2-sulfonyl chloride 81.2: Sulfonyl chloride 81.2 was synthesized from Intermediate 81.1 following the procedure described in Method AB. The crude sulfonyl chloride was used immediately in the forwarding step without further purification.

Synthesis of (R)—N-(2,2,2-trifluoro-1-(4-fluorophenyl)ethyl)pyrazolo[1,5-a]pyrimidine-2-sulfonamide 81.3: Intermediate 81.3 was synthesized from sulfonyl chloride 81.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to get the compound 81.3 as colorless gum (90 mg, 24%). ¹H NMR (400 MHz, DMSO-d₆): δ 9.70 (d, J=10.3 Hz, 1H), 9.02 (d, J=7.0 Hz, 1H), 8.70-8.60 (m, 1H), 7.50-7.40 (m, 2H), 7.25-7.15 (m, 1H), 6.98 (d, J=8.5 Hz, 2H), 6.88 (s, 1H), 5.40-5.30 (s, 1H).

Synthesis of (R)—N-ethyl-N-(2,2,2-trifluoro-1-(4-fluorophenyl)ethyl)pyrazolo[1,5-a]pyrimidine-2-sulfonamide (Example 148): The ethylation of intermediate 81.3 was preformed following the protocol as described in Method AD. The crude residue was purified by column chromatography over silica gel using 1:4 ethyl acetate-hexane to isolate Example 148 as a colorless oil (30 mg, 34% yield, 94.65% purity). LCMS: m/z found 403.26 [M+H]⁺, rt=3.03 min (Method 2) [Waters Acquity BEH C8 column (1.7 μm, 50×2.1 mm)]. ¹H NMR (400 MHz, DMSO-d₆): δ 9.29-9.23 (m, 1H), 8.75 (dd, J=1.7, 4.1 Hz, 1H), 7.46 (dd, J=5.3, 8.7 Hz, 2H), 7.32 (dd, J=4.1, 7.1 Hz, 1H), 7.22-7.11 (m, 3H), 5.96 (q, J=8.7 Hz, 1H), 3.49-3.35 (m, 1H), 3.34-3.21 (m, 1H), 1.02 (t, J=7.0 Hz, 3H).

### Example 149

### (R)—N-ethyl-N-(2,2,2-trifluoro-1-(4-fluorophenyl)ethyl)-1H-pyrrolo[3,2-b]pyridine-6-sulfonamide

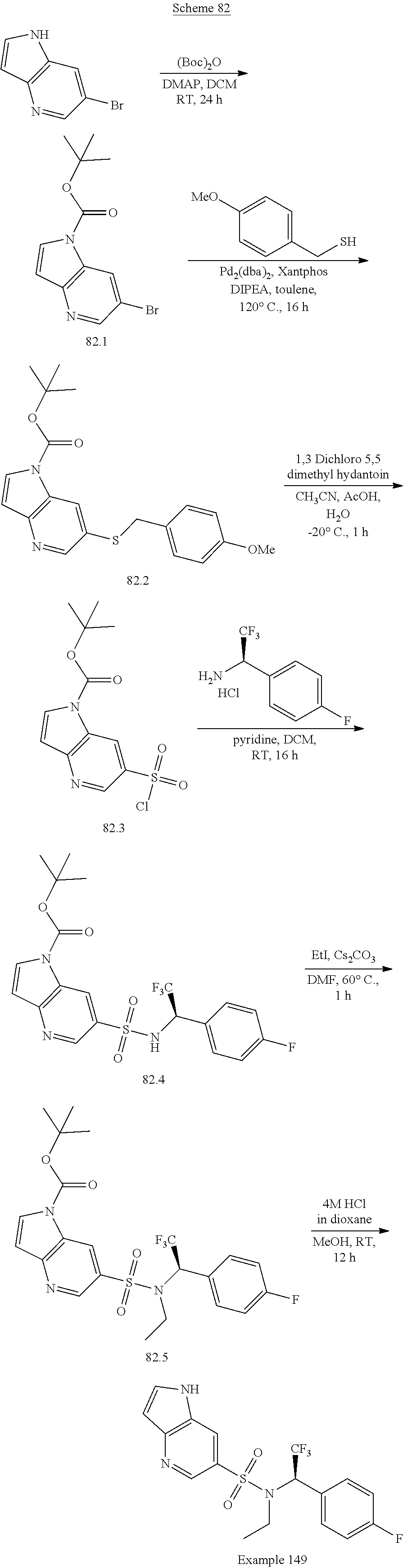

Synthesis of tert-butyl 6-bromo-1H-pyrrolo[3,2-b]pyridine-1-carboxylate 82.1. Intermediate 82.1 was synthesized from 6-bromo-1H-pyrrolo[3,2-b]pyridine following a method analogous to that described in Method AF. The crude was purified by column chromatography over silica gel using 20% ethyl acetate:hexane to get the compound 82.1 as a yellow gum (1.5 g, 99% yield). ¹H NMR (400 MHz, DMSO-d₆): δ 8.68 (s, 1H), 8.24 (s, 1H), 7.26-7.21 (m, 1H), 6.68-6.63 (m, 1H), 1.63 (s, 9H).

Synthesis of tert-butyl 6-((4-methoxybenzyl)thio)-1H-pyrrolo[3,2-b]pyridine-1-carboxylate 82.2: Intermediate 82.2 was synthesized from intermediate 82.1 following a method analogous to that described in Method AA using (4-methoxyphenyl)methanethiol. The crude was purified by column chromatography over silica gel using 20% ethyl acetate:hexane and the compound 82.2 was isolated as a yellow gum (1.2 g, 64% yield). ¹H NMR (400 MHz, DMSO-d₆): δ 8.42 (s, 1H), 8.16 (s, 1H), 7.95 (d, J=3.6 Hz, 1H), 7.17 (d, J=8.4 Hz, 2H), 6.83-6.79 (m, 3H), 4.17 (s, 2H), 3.70 (s, 3H), 1.60 (s, 9H).

Synthesis of tert-butyl 6-(chlorosulfonyl)-1H-pyrrolo[3,2-b]pyridine-1-carboxylate 82.3: Sulfonyl chloride 82.3 was synthesized from Intermediate 82.2 following the procedure described in Method AB. The crude sulfonyl chloride was used immediately in the forwarding step without further purification.

Synthesis of tert-butyl (R)-6-(N-(2,2,2-trifluoro-1-(4-fluorophenyl)ethyl)sulfamoyl)-1H-pyrrolo[3,2-b]pyridine-1-carboxylate 82.4: Intermediate 82.4 was synthesized from sulfonyl chloride 82.3 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate intermediate 82.4 as a yellow sticky gum (150 mg, 25% yield). ¹H NMR (400 MHz, DMSO-d₆): δ 8.91 (s, 1H), 8.43 (s, 1H), 8.23 (bs, 1H), 7.26-7.19 (m, 5H), 6.03 (s, 1H), 6.01 (m, 1H), 1.60 (s, 9H).

Synthesis of tert-butyl (R)-6-(N-ethyl-N-(2,2,2-trifluoro-1-(4-fluorophenyl)ethyl)sulfamoyl)-1H-pyrrolo[3,2-b]pyridine-1-carboxylate 82.5: The ethylation of intermediate 82.4 was preformed following the protocol as described in Method AD. The crude was purified by column chromatography over silica gel using 5:1 ethyl acetate:hexane to obtain the compound 82.5 as a colorless gum (80 mg, 58% Yield). ¹H NMR (400 MHz, DMSO-d₆): δ 8.91 (s, 1H), 8.43 (s, 1H), 8.23 (bs, 1H), 7.26-7.19 (m, 5H), 6.03 (s, 1H), 6.01 (m, 1H), 2.59 (m, 2H), 1.60 (s, 9H), 1.06 (t, J=4.6 Hz, 3H).

Synthesis of (R)—N-ethyl-N-(2,2,2-trifluoro-1-(4-fluorophenyl)ethyl)-1H-pyrrolo[3,2-b]pyridine-6-sulfonamide (Example 149): N-Boc deprotection of intermediate 82.4 was performed following a method analogous to that described in Method AG. The volatiles were evaporated under reduced pressure and the crude was basified with aqueous 10% NaOH solution. It was extracted DCM (2×10 mL), combine organic layers was washed with brine (10 mL), dried over anhydrous sodium sulphate and the solvent was evaporated under reduced pressure. The crude was purified by column chromatography over silica gel using 20% ethyl acetate in hexane to obtain the product Example 149 as a white solid (25 mg, 39% Yield, 98.84% purity). LCMS: m/z found 402.1 [M+H]⁺, rt=2.70 min (Method 9) [Waters Xbridge C18 column (3.5 μm, 50×3 mm)]; ¹H NMR (400 MHz, DMSO-d₆): δ 11.85 (s, 1H), 8.79 (s, 1H), 8.26 (s, 1H), 7.99 (s, 1H), 7.51-7.42 (m, 2H), 7.20 (t, J=8.4 Hz, 2H), 6.72 (s, 1H), 6.09 (q, J=9.7 Hz, 1H), 3.25-3.14 (m, 1H), 0.91 (t, J=6.4 Hz, 3H).

### Example 150

### (R)—N-ethyl-N-(2,2,2-trifluoro-1-(4-fluorophenyl)ethyl)imidazo[1,2-a]pyrazine-2-sulfonamide

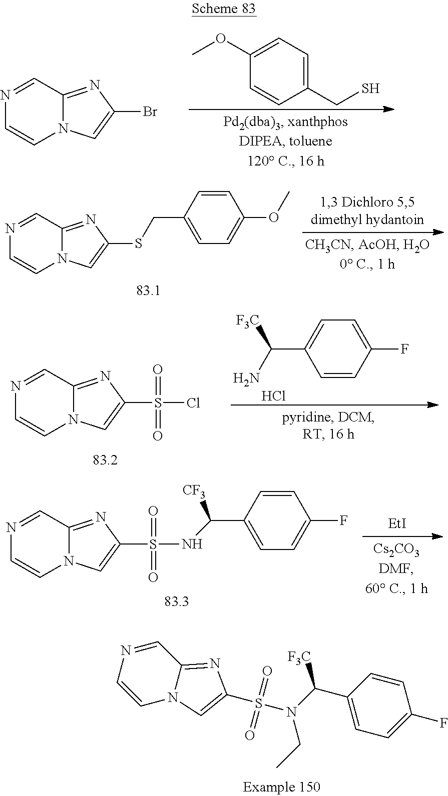

Synthesis of 2-((4-methoxybenzyl)thio)imidazo[1,2-a]pyrazine 83.1: Intermediate 83.1 was synthesized from 2-bromoimidazo[1,2-a]pyrazine following a method analogous to that described in Method AA using (4-methoxyphenyl)methanethiol. The crude was purified by column chromatography over silica gel using 50% ethyl acetate in hexane to yield compound 83.1 as yellow gum (900 mg, 65% yield). LCMS: m/z found 272.0 [M+H]⁺, rt=1.68 min (Method 3) [YMC Triart C18 column (3 μm, 33×2.1 mm)].

Synthesis of imidazo[1,2-a]pyrazine-2-sulfonyl chloride 83.2: Sulfonyl chloride 83.2 was synthesized from Intermediate 83.1 following the procedure described in Method AB. The crude sulfonyl chloride was used immediately in the forwarding step without further purification.

Synthesis of (R)—N-(2,2,2-trifluoro-1-(4-fluorophenyl)ethyl)imidazo[1,2-a]pyrazine-2-sulfonamide 83.3: Intermediate 83.3 was synthesized from sulfonyl chloride 83.2 following the protocol as described in Method AC using (R)-2,2,2-trifluoro-1-(4-fluorophenyl)ethan-1-amine hydrochloride. The compound was purified by column chromatography over silica gel using 70% ethyl acetate in hexane to isolate the compound 83.2 as a yellow sticky gum (200 mg, 23% yield). LCMS: m/z found 375.0 [M+H]⁺, rt=1.67 min (Method 3) [YMC Triart C18 column (3 μm, 33×2.1 mm)].

Synthesis of (R)—N-ethyl-N-(2,2,2-trifluoro-1-(4-fluorophenyl)ethyl)imidazo[1,2-a]pyrazine-2-sulfonamide (Example 150): The ethylation of intermediate 83.3 was performed following the protocol as described in Method AD. The crude was purified by Reverse Phase Prep-HPLC and the compound Example 150 was obtained as a white solid (10 mg, 4% yield, 99.36% purity) after lyophilization. RP method: Preparative HPLC Was done in WATERS BGM 2545 equipped with PDA detector set to multiple-wavelength UV (200-400 nm) detection. Column name: LYMC C18 (250×20 mm, 5μ) operating at ambient temperature and flow rate of 16 mL/min. Mobile phase: A=20 mM ammonium bicarbonate in water, Mobile phase B=mcetonitrile. Gradient profile: Mobile phase initial composition of 50% A and 50% B min, then to 10% A and 90% B in 18 min, then to 0% A and 100% B in 19 min, held in this composition up to 22 min for column washing, then returned to initial composition in 22.5 min and held till 25 min. LCMS: m/z found 403.18 [M+H]⁺, rt=2.95 min (Method 2) [Waters Acquity BEH C8 column (1.7 μm, 50×2.1 mm)]; ¹H NMR (400 MHz, DMSO-d₆): δ 9.33 (s, 1H), 8.76 (d, J=4.9 Hz, 1H), 8.50 (s, 1H), 8.20 (d, J=4.3 Hz, 1H), 7.55-7.36 (m, 2H), 7.20 (t, J=9.0 Hz, 2H), 6.12 (d, J=7.3 Hz, 1H), 3.60-3.40 (m, 2H), 0.87 (t, J=7.0 Hz, 3H).

### Example 151

### (R)—N-ethyl-N-(2,2,2-trifluoro-1-(4-fluorophenyl)ethyl)thiazolo[4,5-b]pyridine-6-sulfonamide

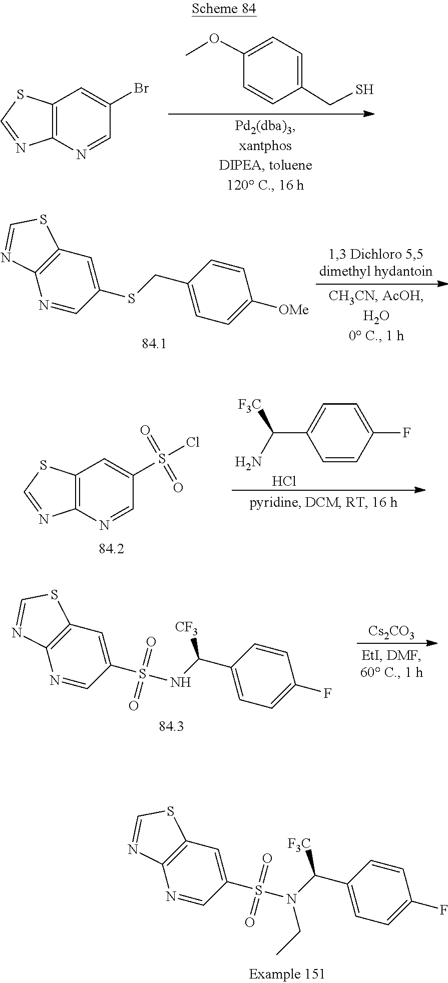

Synthesis of 6-((4-methoxybenzyl)thio)thiazolo[4,5-b]pyridine 84.1: Intermediate 84.1 was synthesized from 6-bromothiazolo[4,5-b]pyridine following a method analogous to that described in Method AA using (4-methoxyphenyl)methanethiol. The crude was purified by column chromatography over silica gel using 40% ethyl acetate in hexane to yield the compound as yellow gum (1.0 g, 74% yield). LCMS: m/z found 289.0 [M+H]⁺, rt=1.79 min (Method 3) [YMC Triart C18 column (3 μm, 33×2.1 mm)].

Synthesis of thiazolo[4,5-b]pyridine-6-sulfonyl chloride 84.2: Sulfonyl chloride 84.2 was synthesized from intermediate 84.1 following the procedure described in Method AB. The crude sulfonyl chloride was used immediately in the forwarding step without further purification.

Synthesis of (R)—N-(2,2,2-trifluoro-1-(4-fluorophenyl)ethyl)thiazolo[4,5-b]pyridine-6-sulfonamide 84.3: Intermediate 84.3 was synthesized from sulfonyl chloride 84.2 following the protocol as described in Method AC using (R)-2,2,2-trifluoro-1-(4-fluorophenyl)ethan-1-amine hydrochloride. The crude compound was used in the forwarding step without further purification.

Synthesis of (R)—N-ethyl-N-(2,2,2-trifluoro-1-(4-fluorophenyl)ethyl)thiazolo[4,5-b]pyridine-6-sulfonamide (Example 151): The ethylation of intermediate 84.3 was performed following the protocol as described in Method AD. The crude was purified by Reverse Phase Prep-HPLC and the compound Example 151 was obtained as an off-white solid (10 mg, 4% yield, 98.01% purity) after lyophilization. RP method: Preparative HPLC Was done in WATERS BGM 2545 equipped with PDA detector set to multiple-wavelength UV (200-400 nm) detection. Column name: XBridge C18 (50×19 mm, 5μ) operating at ambient temperature and flow rate of 24 mL/min. Mobile phase: A=20 mM ammonium bicarbonate in water, Mobile phase B=acetonitrile. Gradient profile: Mobile phase initial composition of 90% A and 10% B min, then to 65% A and 35% B in 1 min, then to 30% A and 70% B in 7 min, then to 0% A and 100% B in 9 min, held in this composition up to 12 min for column washing, then returned to initial composition in 12.5 min and held till 15 min. LCMS: m/z found 420.15 [M+H]⁺, rt=3.01 min (Method B) [Waters Acquity BEH C8 column (1.7 μm, 50×2.1 mm)]; ¹H NMR (400 MHz, DMSO-d₆): δ 9.97 (s, 1H), 9.29 (d, J=1.6 Hz, 1H), 9.20 (d, J=2.4 Hz, 1H), 7.47 (dd, J=5.2, 8.6 Hz, 2H), 7.20 (t, J=8.8 Hz, 2H), 6.08 (q, J=8.8 Hz, 1H), 3.48-3.31 (m, 2H), 0.99 (t, J=6.8 Hz, 3H).

### Example 152

### (R)—N-ethyl-N-(2,2,2-trifluoro-1-(4-fluorophenyl)ethyl)-[1,2,4]triazolo[4,3-c]pyrimidine-7-sulfonamide

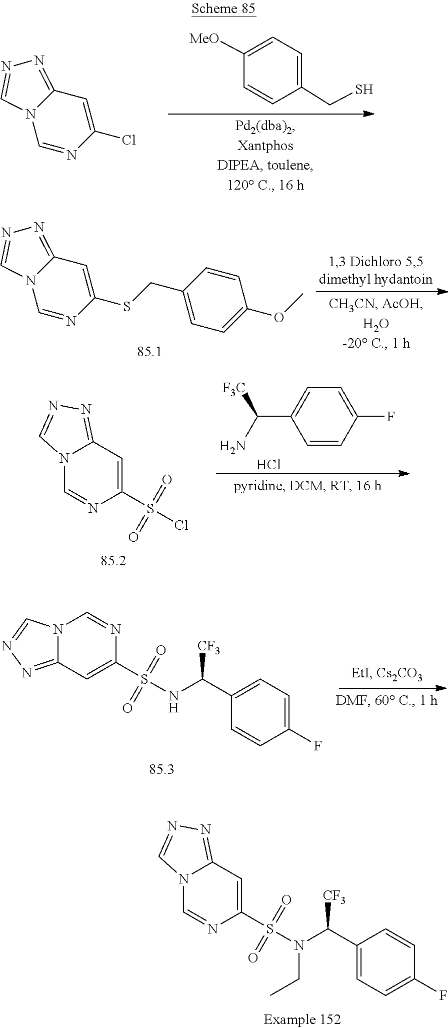

Synthesis of 7-((4-methoxybenzyl)thio)-[1,2,4]triazolo[4,3-c]pyrimidine 85.1: Intermediate 85.1 was synthesized from 7-chloro-[1,2,4]triazolo[4,3-c]pyrimidine following a method analogous to that described in Method AA using (4-methoxyphenyl)methanethiol. The crude was purified by column chromatography over silica gel using 50% ethyl acetate in hexane and to yield the compound 85.1 as a yellow gum (450 mg, 51% yield). LCMS: m/z found 272.08 [M+H]⁺, rt=1.92 min (Method 4) [Waters Xbridge C18 column (5 μm, 50×4.6 mm)].

Synthesis of [1,2,4]triazolo[4,3-c]pyrimidine-7-sulfonyl chloride 85.2. Sulfonyl chloride 85.2 was synthesized from Intermediate 85.1 following the procedure described in Method AB. The crude sulfonyl chloride was used immediately in the forwarding step without further purification.

Synthesis of (R)—N-(2,2,2-trifluoro-1-(4-fluorophenyl)ethyl)-[1,2,4]triazolo[4,3-c]pyrimidine-7-sulfonamide 85.3: Intermediate 85.3 was synthesized from sulfonyl chloride 85.2 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate the compound 85.2 as yellow sticky gum (100 mg, 42% yield). ¹H NMR (400 MHz, DMSO-d₆): δ 9.93 (d, J=9.6 Hz, 1H), 9.81 (s, 1H), 8.86 (s, 1H), 8.27 (s, 1H), 7.48-7.41 (m, 2H), 7.00 (t, J=8.2 Hz 2H), 5.39 (t, J=9.7 Hz, 1H).

Synthesis of R)—N-ethyl-N-(2,2,2-trifluoro-1-(4-fluorophenyl)ethyl)-[1,2,4]triazolo[4,3-c]pyrimidine-7-sulfonamide (Example 152): The ethylation of intermediate 85.3 was preformed following the protocol as described in Method AD. The crude was purified by column chromatography over silica gel using 1:4 ethyl acetate-hexane to yield the compound Example 152 as a white solid (50 mg, 46% yield, 98.75% purity). LCMS: m/z found 404.17 [M+H]⁺, rt=3.05 min (Method 2) [Waters Acquity BEH C8 column (1.7 μm, 50×2.1 mm)]; ¹H NMR (400 MHz, Chloroform-d): δ 9.43 (s, 1H), 8.60 (s, 1H), 8.39 (s, 1H), 7.57 (t, J=6.4 Hz, 2H), 7.10 (t, J=8.4 Hz, 2H), 5.79 (q, J=8.0 Hz, 1H), 3.66-3.54 (m, 1H), 3.53-3.42 (m, 1H), 0.99 (t, J=6.8 Hz, 3H).

### Example 153

### (R)—N-ethyl-N-(2,2,2-trifluoro-1-(4-fluorophenyl)ethyl)-[1,2,4]triazolo[1,5-a]pyridine-2-sulfonamide

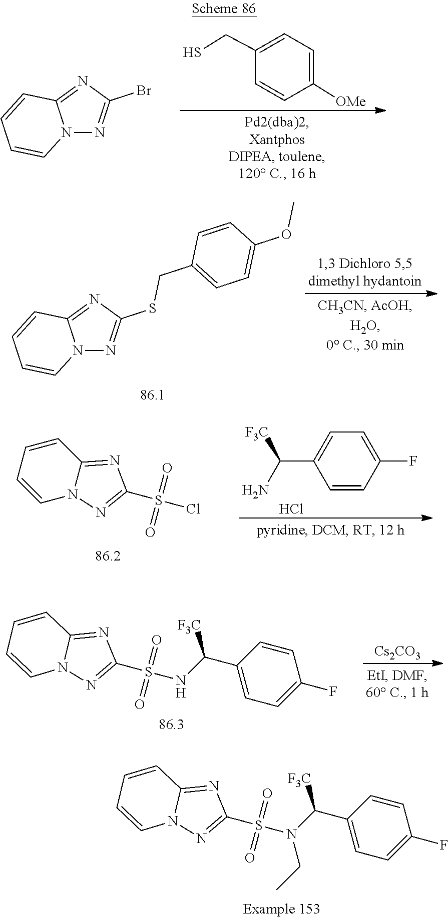

Synthesis of 2-((4-methoxybenzyl)thio)-[1,2,4]triazolo[1,5-a]pyridine 86.1: Intermediate 86.1 was synthesized from 2-bromo-[1,2,4]triazolo[1,5-a]pyridine following a method analogous to that described in Method AA using (4-methoxyphenyl)methanethiol. The crude residue was purified by column chromatography over silica gel using 20% ethyl acetate in hexane to get the compound 86.1 as yellow gum. (200 mg, 58% yield). ¹H NMR (400 MHz, DMSO-d₆): δ 8.65-8.55 (m, 2H), 7.74 (s, 1H), 7.10-7.05 (m, 2H), 6.94 (d, J=8.5 Hz, 2H), 6.72 (d, J=8.6 Hz, 1H), 3.92 (s, 2H), 3.67 (s, 3H).

Synthesis of [1,2,4]triazolo[1,5-a]pyridine-2-sulfonyl chloride 86.2: Intermediate 86.1 (450 mg) was dissolved in DCM (10 mL) and water (1 mL) was added to it. The reaction mixture was cooled at −5° C. and Cl₂ gas was bubbled through it for 30 mins. The reaction mixture was diluted with ice water (5 mL) and extracted with DCM (5 mL). The organic part was dried over anhydrous Na₂SO₄ and filtered. The filtrate was directly used in the forwarding step without evaporating.

Synthesis of (R)—N-(2,2,2-trifluoro-1-(4-fluorophenyl)ethyl)-[1,2,4]triazolo[1,5-a]pyridine-2-sulfonamide 86.3: Intermediate 86.3 was synthesized from sulfonyl chloride 86.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to yield the compound 86.3 as colorless gum (170 mg, 28%). ¹H NMR (400 MHz, DMSO-d₆): δ 10.01 (d, J=10.0 Hz, 1H), 8.88 (d, J=6.7 Hz, 1H), 7.82-7.75 (m, 2H), 7.50-7.40 (m, 2H), 7.36-7.30 (m, 1H), 6.97 (t, J=8.6 Hz, 2H), 5.40-5.30 (m, 1H).

Synthesis of (R)—N-ethyl-N-(2,2,2-trifluoro-1-(4-fluorophenyl)ethyl)-[1,2,4]triazolo[1,5-a]pyridine-2-sulfonamide (Example 153): The ethylation of intermediate 86.3 was preformed following the protocol as described in Method AD. The crude was purified by column chromatography over silica gel using 1:4 ethyl acetate-hexane to yield Example 153 as a colorless gum (120 mg, 60% yield, 98.63% purity). LCMS: m/z found 403.18 [M+H]⁺, rt=3.06 min (Method B) [Waters Acquity BEH C8 column (1.7 μm, 50×2.1 mm)]; ¹H NMR (400 MHz, DMSO-d₆): δ 9.07 (d, J=6.8 Hz, 1H), 7.99 (d, J=8.8 Hz, 1H), 7.87 (t, J=8.4 Hz, 1H), 7.50 (dd, J=5.2, 8.6 Hz, 2H), 7.43 (t, J=6.8 Hz, 1H), 7.15 (t, J=8.8 Hz, 2H), 5.92 (q, J=8.4 Hz, 1H), 3.56-3.34 (m, 2H), 1.01 (t, J=7.2 Hz, 3H).

### Example 154

### (R)-2-amino-N-ethyl-N-(2,2,2-trifluoro-1-(4-fluorophenyl)ethyl)benzo[d]thiazole-6-sulfonamide

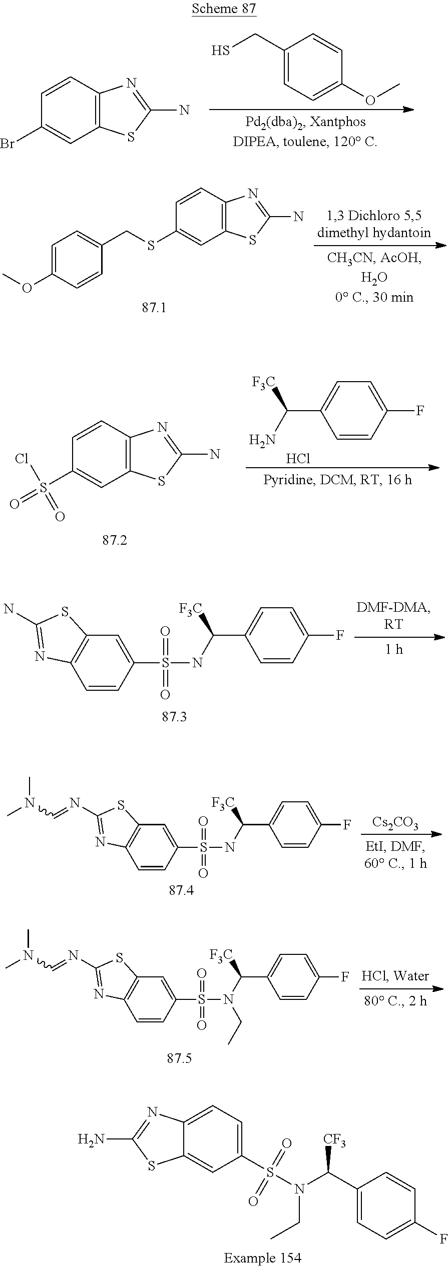

Synthesis of 6-((4-methoxybenzyl)thio)benzo[d]thiazol-2-amine 87.1: Intermediate 87.1 was synthesized from 6-bromobenzo[d]thiazol-2-amine following a method analogous to that described in Method AA using (4-methoxyphenyl)methanethiol. The volatiles were evaporated under reduced pressure and triturated with hexane to get 6-((4-methoxybenzyl)thio)benzo[d]thiazol-2-amine. The compound was used in the following step without further purification. LCMS: m/z found 403.1 [M+H]⁺, rt=1.72 min (Method 3) [YMC Triart C18 column (3 μm, 33×2.1 mm)].

Synthesis of 2-aminobenzo[d]thiazole-6-sulfonyl chloride 87.2: Sulfonyl chloride 87.2 was synthesized from intermediate 87.1 following the procedure described in Method AB. The crude sulfonyl chloride was used immediately in the forwarding step without further purification.

Synthesis of (R)-2-amino-N-(2,2,2-trifluoro-1-(4-fluorophenyl)ethyl)benzo[d]thiazole-6-sulfonamide 87.3: Intermediate 87.3 was synthesized from 2-aminobenzo[d]thiazole-6-sulfonyl chloride following the protocol as described in Method AC using (R)-2,2,2-trifluoro-1-(4-(trifluoromethyl)phenyl)ethan-1-amine hydrochloride. The compound was purified by column chromatography over silica gel using 35% ethyl acetate in hexane to isolate the compound 87.3 as a yellow sticky gum (200 mg, 20% yield). LCMS: m/z found 406.0 [M+H]⁺, rt=1.72 min (Method 3) [YMC Triart C18 column (3 μm, 33×2.1 mm)].

Synthesis of (R)—N,N-dimethyl-N′-(6-(N-(2,2,2-trifluoro-1-(4-fluorophenyl)ethyl)sulfamoyl)benzo[d]thiazol-2-yl)formimidamide 87.4: N-DMF-DMA protection: Method AH: To the stirred solution of intermediate 87.3 (200 mg) in DMF (1 ml) DMF-DMA (2 mL) was added at RT and the mixture was stirred for 2 h at RT. The reaction mixture was diluted with water (15 mL), then extracted with DCM (2×15 mL), the combined organic layers were washed with water (2×10 mL), dried over anhydrous Na₂SO₄ and the solvent was evaporated under reduced pressure. The crude residue was purified by column chromatography over silica gel using 20% ethyl acetate in hexane to provide compound 87.4 as an off-white solid (120 mg, 69% yield). LCMS: m/z found 461.2 [M+H]⁺, rt=2.12 min (Method 12) [YMC Triart C18 column (3 μm, 33×2.1 mm)].

(R)—N′-(6-(N-ethyl-N-(2,2,2-trifluoro-1-(4-fluorophenyl)ethyl)sulfamoyl)benzo[d]thiazol-2-yl)-N,N-dimethylformimidamide 87.5: The ethylation of intermediate 87.4 was performed following the protocol as described in Method AD. The crude was purified by column chromatography over silica gel using 5:1 ethyl acetate-hexane to get the compound 87.5 as white solid. LCMS: m/z found 489.1 [M+H]⁺, rt=2.12 min (Method 3) [YMC Triart C18 column (3 μm, 33×2.1 mm)].

(R)-2-amino-N-ethyl-N-(2,2,2-trifluoro-1-(4-fluorophenyl)ethyl)benzo[d]thiazole-6-sulfonamide (Example 154): N-DMF-DMA deprotection: Method AI: To the stirred solution of intermediate 87.5 (50 mg, 0.08 mmol) in EtOH (1 ml) and water (2 ml), conc. HCl (1 mL) was added at RT and the mixture was stirred for 2 h at 80° C. The reaction mixture was then cooled and diluted with water (5 mL). The mixture was then extracted with DCM (3×5 mL), the combined organic layers were washed with water (10 mL), dried over anhydrous Na₂SO₄, and the solvent was evaporated under reduced pressure. The crude residue was purified by column chromatography over silica gel using 30% ethyl acetate in hexane to obtain compound Example 154 was isolated as a white solid (95.70% purity). LCMS: m/z found 434.18 [M+H]⁺, rt=3.01 min (Method 2) [Waters Acquity BEH C8 column (1.7 μm, 50×2.1 mm)]; ¹H NMR (400 MHz, DMSO-d₆): δ 8.23 (d, J=1.8 Hz, 1H), 7.99 (s, 2H), 7.70 (dd, J=2.0, 8.5 Hz, 1H), 7.47-7.37 (m, 3H), 7.20 (t, J=8.8 Hz, 2H), 5.92 (q, J=8.8 Hz, 1H), 3.33-3.20 (m, 1H), 3.20-3.05 (m, 1H), 0.92 (t, J=7.0 Hz, 3H).

### Example 155

### (R)-2-amino-N-ethyl-N-(2,2,2-trifluoro-1-(4-fluorophenyl)ethyl)benzo[d]thiazole-5-sulfonamide

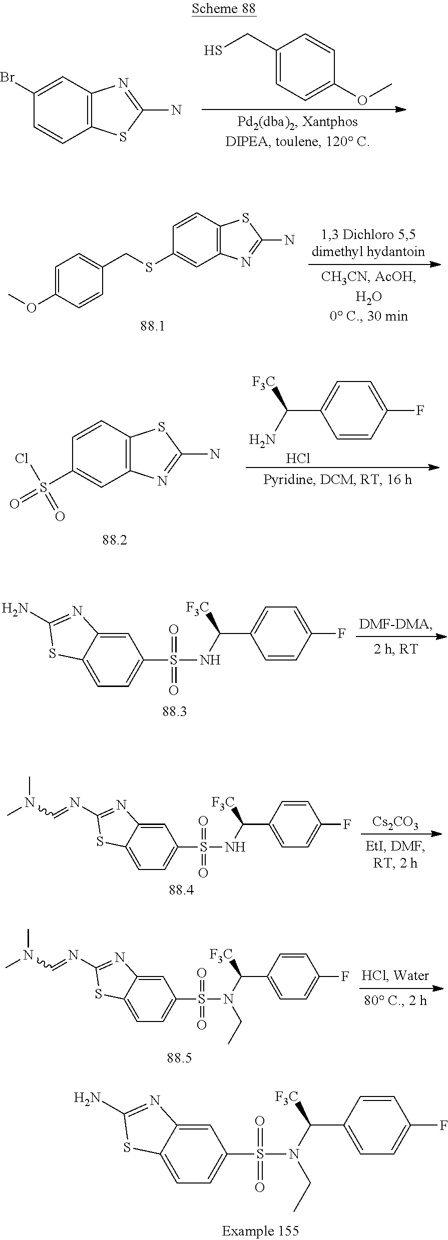

Synthesis of 5-((4-methoxybenzyl)thio)benzo[d]thiazol-2-amine 88.1: Intermediate 88.1 was synthesized from 5-bromobenzo[d]thiazol-2-amine following a method analogous to that described in Method AA using (4-methoxyphenyl)methanethiol. The volatiles were evaporated under reduced pressure and triturated with hexane to get 5-((4-methoxybenzyl)thio)benzo[d]thiazol-2-amine. This compound was used in the following step without further purification. LCMS: m/z found 303.0 [M+H]⁺, rt=1.75 min (Method 3) [YMC Triart C18 column (3 μm, 33×2.1 mm)].

Synthesis of 2-aminobenzo[d]thiazole-5-sulfonyl chloride 88.2: Sulfonyl chloride 88.2 was synthesized from intermediate 88.1 following the procedure described in Method AB. The crude sulfonyl chloride was used immediately in the forwarding step without further purification.

Synthesis of (R)-2-amino-N-(2,2,2-trifluoro-1-(4-fluorophenyl)ethyl)benzo[d]thiazole-5-sulfonamide 88.3: Intermediate 88.3 was synthesized from 2-aminobenzo[d]thiazole-5-sulfonyl chloride 88.2 following the protocol as described in Method AC using (R)-2,2,2-trifluoro-1-(4-(trifluoromethyl)phenyl)ethan-1-amine hydrochloride. The compound was purified by column chromatography over silica gel using 35% ethyl acetate in hexane to isolate as a yellow sticky gum (300 mg, 30% yield). LCMS: m/z found 405.88 [M+H]⁺, rt=1.80 min (Method 15) [Waters Acquity BEH C18 column (1.7 μm, 50×2.1 mm)].

Synthesis of (R)—N,N-dimethyl-N′-(5-(N-(2,2,2-trifluoro-1-(4-fluorophenyl)ethyl)sulfamoyl)benzo[d]thiazol-2-yl)formimidamide 88.4: N-DMF-DMA protection of intermediate 88.3 was performed following a method analogous to that described in Method AH. The crude residue was purified by column chromatography over silica gel using 20% ethyl acetate in hexane to yield the compound 88.4 as an off-white solid (200 mg, 58% yield). LCMS: m/z found 461.0 [M+H]⁺, rt=1.84 min (Method 3) [YMC Triart C18 column (3 μm, 33×2.1 mm)].

Synthesis of (R)—N′-(5-(N-ethyl-N-(2,2,2-trifluoro-1-(4-fluorophenyl)ethyl)sulfamoyl)benzo[d]thiazol-2-yl)-N,N-dimethylformimidamide 88.5: The ethylation of intermediate 88.4 was preformed following the protocol as described in Method AD. The crude was purified by column chromatography over silica gel using 5:1 ethyl acetate:hexane and the compound was obtained as a white solid (50 mg, 17% yield). LCMS: m/z found 489.1 [M+H]⁺, rt=1.98 min (Method 3) [YMC Triart C18 column (3 μm, 33×2.1 mm)].

Synthesis of (R)-2-amino-N-ethyl-N-(2,2,2-trifluoro-1-(4-fluorophenyl)ethyl)benzo[d]thiazole-5-sulfonamide (Example 155): N-DMF-DMA deprotection of intermediate 88.5 was performed following a method analogous to that described in Method AI. The crude was purified by column chromatography over silica gel using 30% ethyl acetate:hexane to isolate compound Example 155 as white solid (96.46% purity). LCMS: m/z found 434.12 [M+H]⁺, rt=3.02 min (Method 2) [Waters Acquity BEH C8 column (1.7 μm, 50×2.1 mm)]; ¹H NMR (400 MHz, DMSO-d₆): δ 7.84 (d, J=8.0 Hz, 1H), 7.72 (d, J=1.2 Hz, 1H), 7.54 (s, 2H), 7.52-7.42 (m, 3H), 7.19 (t, J=8.8 Hz, 2H), 5.90 (q, J=8.8 Hz, 1H), 3.41-3.15 (m, 2H), 0.94 (t, J=6.8 Hz, 3H).

### Example 156

### (R)—N-ethyl-N-(2,2,2-trifluoro-1-(4-fluorophenyl)ethyl)-1H-imidazo[4,5-b]pyridine-6-sulfonamide

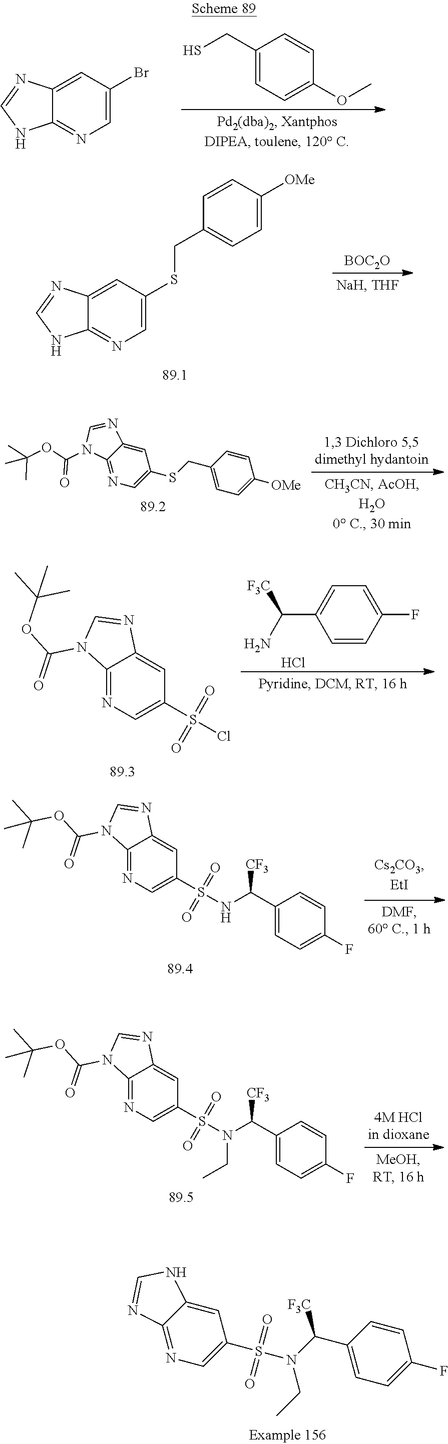

Synthesis of 6-((4-methoxybenzyl)thio)-3H-imidazo[4,5-b]pyridine 89.1: Intermediate 89.1 was synthesized from 6-bromo-3H-imidazo[4,5-b]pyridine following a method analogous to that described in Method AA using (4-methoxyphenyl)methanethiol. The crude residue was purified by combi flash column chromatography using 50% ethyl acetate in hexane as eluent to afford product 89.1 (1.1 g, 80% yield) as a yellow oil. LCMS: m/z found 272.0 [M+H]⁺, rt=1.59 min (Method 3) [YMC Triart C18 column (3 μm, 33×2.1 mm)].

Synthesis of tert-butyl 6-((4-methoxybenzyl)thio)-3H-imidazo[4,5-b]pyridine-3-carboxylate 89.2: To a stirred solution of intermediate 89.1 (1.0 g, 3.67 mmol) in THF (10 mL) NaH (245 mg, 7.35 mmol) was added portionwise at 0° C. and the reaction mixture was stirred for 30 min. Boc₂O (1.23 mL, 5.51 mmol) was added to it and the reaction mixture was stirred for 2 h. The reaction was quenched with a saturated NH₄Cl solution (25 mL) and extracted with ethyl acetate (2×25 mL). The combined organic layer washed with water (25 mL) and brine (25 mL), dried over anhydrous Na₂SO₄ and concentrated under reduced pressure. The crude residue was purified by combiflash column chromatography using 20% ethyl acetate in hexane as eluent to afford desire product 89.2 (750 mg, 55% yield). LCMS: m/z found 372.2 [M+H]⁺, rt=2.02 min (Method 8) [Waters Xbridge C18 column (3.5 μm, 50×3 mm)].

Synthesis of tert-butyl 6-(chlorosulfonyl)-3H-imidazo[4,5-b]pyridine-3-carboxylate 89.3: Sulfonyl chloride 89.3 was synthesized from intermediate 89.2 following the procedure described in Method AB. The crude sulfonyl chloride was used immediately in the following step without further purification.

Synthesis of tert-butyl (R)-6-(N-(2,2,2-trifluoro-1-(4-fluorophenyl)ethyl)sulfamoyl)-3H-imidazo[4,5-b]pyridine-3-carboxylate 89.4: intermediate 89.4 was synthesized from sulfonyl chloride 89.3 following the protocol as described in Method AC using (R)-2,2,2-trifluoro-1-(4-(trifluoromethyl)phenyl)ethan-1-amine hydrochloride. The crude was purified by column chromatography over silica gel using 35% ethyl acetate in hexane to isolate the compound as a yellow sticky gum (220 mg, 29% yield). LCMS: m/z found 475.1 [M+H]⁺, rt=1.88 min (Method 8) [Waters Xbridge C18 column (3.5 μm, 50×3 mm)].

Synthesis of tert-butyl (R)-6-(N-ethyl-N-(2,2,2-trifluoro-1-(4-fluorophenyl)ethyl)sulfamoyl)-3H-imidazo[4,5-b]pyridine-3-carboxylate 89.5: The ethylation of intermediate 89.4 was preformed following the protocol as described in Method AD. The crude was purified by column chromatography over silica gel using 15% ethyl acetate in hexane as eluent to afford pure product 89.5 (70 mg, 30% yield) as a colourless gum. LCMS: m/z found 503.1 [M+H]⁺, rt=3.78 min (Method D) [Waters Xbridge C18 column (5 μm, 50×4.6 mm).

Synthesis of (R)—N-ethyl-N-(2,2,2-trifluoro-1-(4-fluorophenyl)ethyl)-3H-imidazo[4,5-b]pyridine-6-sulfonamide (Example 156): N-Boc deprotection of intermediate 89.5 was performed following a method analogous to that described in Method AG. The volatiles were evaporated under reduced pressure and the crude was purified by Reverse Phase Prep HPLC to afford Example 156 as a white solid (30 mg, 53% yield, 99.79% purity) after lyophilization. RP method: Preparative HPLC Was done in a Waters BGM 2545 equipped with Waters PDA detector 2998 set to multiple-wavelength UV (200-400 nm) detection. Column name: Xbridge C18 (50×19 mm, 5μ) operating at ambient temperature and flow rate of 24 mL/min. Mobile phase: A=20 mM ammonium bicarbonate in water, B=acetonitrile. Gradient profile: Mobile phase initial composition of 80% A and 20% B, then to 10% A and 90% B in 10 min, held in this composition to 12 min for column washing, then returned to initial composition in 12.5 min and held till 15 min. LCMS: m/z found 402.92 [M+H]⁺, rt=1.84 min (Method AE) [Waters Acquity BEH C18 column (1.7 μm, 50×2.1 mm)]. ¹H NMR (400 MHz, DMSO-d₆): δ 13.81-13.25 (m, 1H), 8.87 (s, 1H), 8.70 (s, 1H), 8.53 (s, 1H), 7.53-7.41 (m, 2H), 7.20 (t, J=8.7 Hz, 2H), 6.17-6.05 (m, 1H), 3.48-3.33 (m, 1H), 3.30-3.17 (m, 1H), 0.94 (t, J=6.8 Hz, 3H).

### Example 157

### (R)-3-chloro-N-ethyl-2-methyl-N-(2,2,2-trifluoro-1-(4-fluorophenyl)ethyl)imidazo[1,2-b]pyridazine-6-sulfonamide

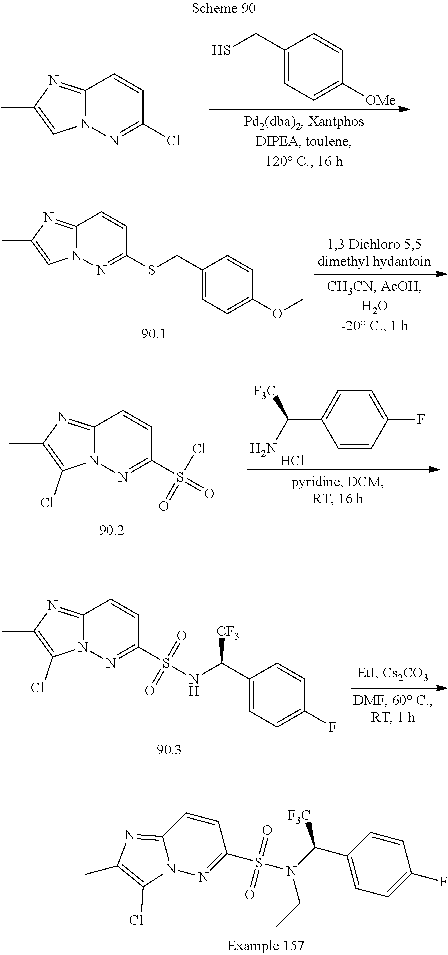

Synthesis of 6-((4-methoxybenzyl)thio)-2-methylimidazo[1,2-b]pyridazine 90.1: Intermediate 90.1 was synthesized from 6-chloro-2-methylimidazo[1,2-b]pyridazine following a method analogous to that described in Method AA using (4-methoxyphenyl)methanethiol. The crude was purified by column chromatography over silica gel using 50% ethyl acetate-hexane to afford the compound as a yellow gum (1.2 g, 35% yield). LCMS: m/z found 286.2 [M+H]⁺, rt=1.61 min (Method 2) [Waters Acquity BEH C8 column (1.7 μm, 50×2.1 mm)]; ¹H NMR (400 MHz, DMSO-d₆): δ 7.99 (s, 1H), 7.82 (d, J=12.2 Hz, 1H), 7.39 (d, J=8.4 Hz, 2H), 7.05 (m, 1H), 6.87 (d, J=8.56 Hz, 2H), 4.31 (s, 2H), 3.71 (s, 3H), 2.34 (s, 3H).

Synthesis of 3-chloro-2-methylimidazo[1,2-b]pyridazine-6-sulfonyl chloride 90.2: Sulfonyl chloride 90.2 was synthesized from intermediate 90.1 following the procedure described in Method AB. The crude sulfonyl chloride was used immediately in the following step without further purification.

Synthesis of (R)-3-chloro-2-methyl-N-(2,2,2-trifluoro-1-(4-fluorophenyl)ethyl)imidazo[1,2-b]pyridazine-6-sulfonamide 90.3: intermediate 90.3 was synthesized from sulfonyl chloride 90.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to get the compound 90.3 as yellow sticky solid (100 mg, 14% yield). ¹H NMR (400 MHz, DMSO-d₆): δ 10.08 (d, J=2.5 Hz, 1H), 8.21 (d, J=12.2 Hz, 1H), 7.55-7.43 (m, 3H), 6.99-6.94 (m, 2H), 5.47 (d, J=8.56 Hz, 1H), 2.42 (s, 3H).

Synthesis of (R)-3-chloro-N-ethyl-2-methyl-N-(2,2,2-trifluoro-1-(4-fluorophenyl)ethyl)imidazo[1,2-b]pyridazine-6-sulfonamide (Example 157): The ethylation of intermediate 90.3 was performed following the protocol as described in Method AD. The crude residue was purified by column chromatography over silica gel using 1:4 ethyl acetate-hexane to afford the compound Example 157 as a white solid (25 mg, 46% yield, 98.81% purity). LCMS: m/z found 451.2 [M+H]⁺, rt=3.27 min (Method 9) [Waters Acquity BEH C8 column (1.7 μm, 50×2.1 mm)]; ¹H NMR (400 MHz, Chloroform-d): δ 8.02 (d, J=9.2 Hz, 1H), 7.65-7.55 (m, 3H), 7.07 (t, J=6.2, 2H), 5.85 (q, J=8.5 Hz, 1H), 3.54-3.47 (m, 2H), 2.57 (s, 3H), 1.01 (t, J=6.8 Hz, 3H).

### Example 158

### (R)—N-ethyl-N-(2,2,2-trifluoro-1-(4-fluorophenyl)ethyl)tetrazolo[1,5-a]pyridine-7-sulfonamide

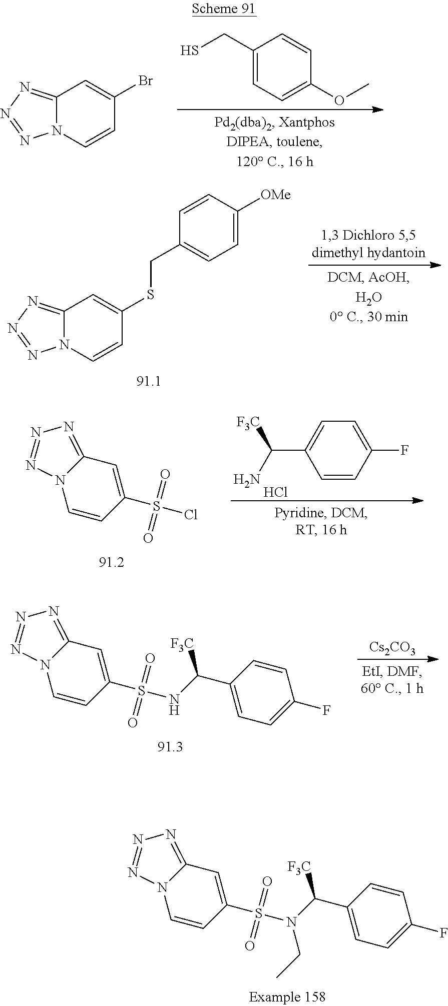

Synthesis of 7-((4-methoxybenzyl)thio)tetrazolo[1,5-a]pyridine 91.1: Intermediate 91.1 was synthesized from 7-bromotetrazolo[1,5-a]pyridine following a method analogous to that described in Method AA using (4-methoxyphenyl)methanethiol. The crude was purified by column chromatography over silica gel using 20% ethyl acetate in hexane to get compound 91a 0.1 as a yellow gum. (150 mg, 40% yield). LCMS: m/z found 273.0 [M+H]⁺, rt=1.78 min (Method 3) [YMC Triart C18 column (3 μm, 33×2.1 mm)].

Synthesis of tetrazolo[1,5-a]pyridine-7-sulfonyl chloride 91.2: Sulfonyl chloride 91.2 was synthesized from intermediate 91.1 following the procedure described in Method AB. The crude sulfonyl chloride was used immediately in the forwarding step without further purification.

Synthesis of (R)—N-(2,2,2-trifluoro-1-(4-fluorophenyl)ethyl)tetrazolo[1,5-a]pyridine-7-sulfonamide 91.3: Intermediate 91.3 was synthesized from sulfonyl chloride 91.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to yield compound 91.3 as a yellow gum (100 mg, 20% yield). LCMS: m/z found 376.0 [M+H]⁺, rt=1.75 min (Method 3) [YMC Triart C18 column (3 μm, 33×2.1 mm)].

Synthesis of (R)—N-ethyl-N-(2,2,2-trifluoro-1-(4-fluorophenyl)ethyl)tetrazolo[1,5-a]pyridine-7-sulfonamide (Example 158): The ethylation of intermediate 91.3 was preformed following the protocol as described in Method AD. The crude was purified by Reverse-Phase Prep-HPLC and the compound was obtained as a white solid (25 mg, 99.35% purity) after lyophilization. LCMS: m/z found 404.28 [M+H]⁺, rt=3.06 min (Method 2) [Waters Acquity BEH C8 column (1.7 μm, 50×2.1 mm)]; ¹H NMR (400 MHz, DMSO-d₆): δ 9.54 (d, J=7.2 Hz, 1H), 8.87 (s, 1H), 7.84 (d, J=7.0 Hz, 1H), 7.51 (dd, J=5.3, 8.5 Hz, 2H), 7.23 (t, J=8.8 Hz, 2H), 6.17 (q, J=8.5 Hz, 1H), 3.55-3.31 (m, 2H), 0.99 (t, J=7.0 Hz, 3H).

### Example 159

### (R)—N-ethyl-N-(2,2,2-trifluoro-1-(4-fluorophenyl)ethyl)-1H-indazole-6-sulfonamide

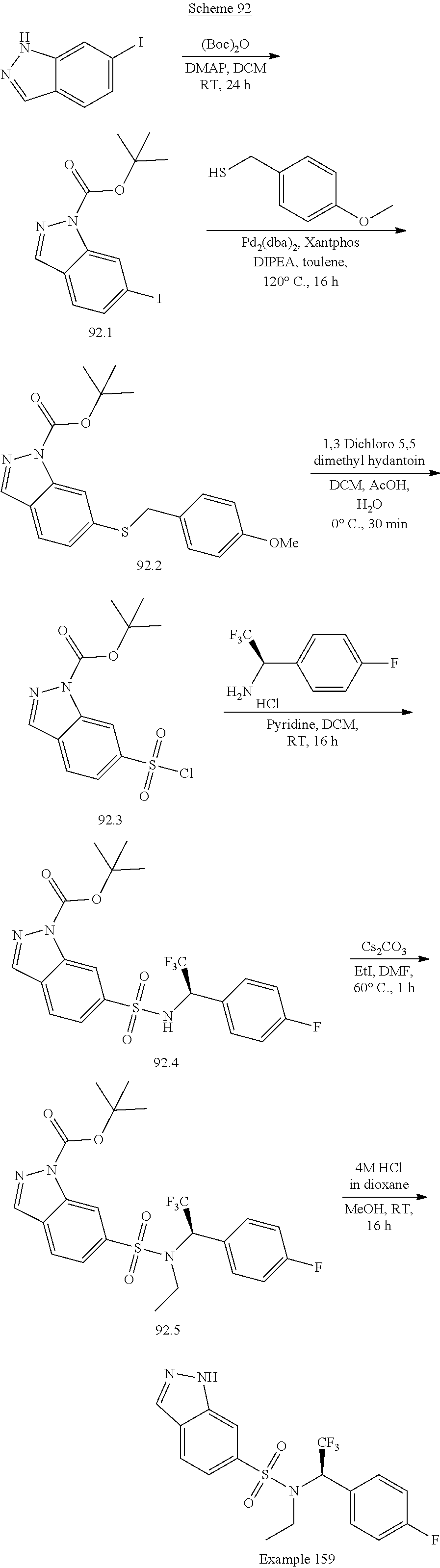

Synthesis of tert-butyl 6-iodo-1H-indazole-1-carboxylate 92.1: Intermediate 92.1 was synthesized from 6-iodo-1H-indazole following a method analogous to that described in Method AF. The crude residue was purified by column chromatography over silica gel using 20% ethyl acetate in hexane to yield the compound 92.1 as a yellow gum (1.3 g, 92% yield). ¹H NMR (400 MHz, DMSO-d₆): δ 8.78 (s, 1H), 8.39 (s, 1H), 7.69 (s, 2H), 1.64 (s, 9H).

Synthesis of tert-butyl 6-((4-methoxybenzyl)thio)-1H-indazole-1-carboxylate 92.2: intermediate 92.2 was synthesized from intermediate 92.1 following a method analogous to that described in Method AA using (4-methoxyphenyl)methanethiol. The crude was purified by column chromatography over silica gel using 20% ethyl acetate i9hexane to isolate the compound 92.2 as yellow gum (1.5 g, 69% yield). ¹H NMR (400 MHz, DMSO-d₆): δ 8.33 (s, 1H), 7.96 (s, 1H), 7.79 (d, J=8.6 Hz, 1H), 7.33-7.29 (m, 3H), 6.87 (d, J=8.6 Hz, 2H), 4.21 (s, 2H), 3.71 (s, 3H), 1.62 (s, 9H).

Synthesis of tert-butyl 6-(chlorosulfonyl)-1H-indazole-1-carboxylate 92.3: Sulfonyl chloride 92.3 was synthesized from Intermediate 92.2 following the procedure described in Method AB. The crude sulfonyl chloride was used immediately in the following step without further purification.

Synthesis of tert-butyl (R)-6-(N-(2,2,2-trifluoro-1-(4-fluorophenyl)ethyl)sulfamoyl)-1H-indazole-1-carboxylate 92.4: intermediate 92.4 was synthesized from sulfonyl chloride 92.3 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate the compound as yellow sticky gum (150 mg, 25% yield).

Synthesis of tert-butyl (R)-6-(N-ethyl-N-(2,2,2-trifluoro-1-(4-fluorophenyl)ethyl)sulfamoyl)-1H-indazole-1-carboxylate 92.5: The ethylation of intermediate 92.4 was preformed following the protocol as described in Method AD. The crude residue was purified by column chromatography over silica gel using 5:1 ethyl acetate:hexane to yield the compound as a colorless gum (80 mg, 21% yield). LCMS: m/z found 502.2 [M+H]⁺, rt=1.96 min (Method 4) [Waters Xbridge C18 column (5 μm, 50×4.6 mm)].

Synthesis of (R)—N-ethyl-N-(2,2,2-trifluoro-1-(4-fluorophenyl)ethyl)-1H-indazole-6-sulfonamide (Example 159): N-Boc deprotection of intermediate 92.4 was performed following a method analogous to that described in Method AG. The volatiles were evaporated under reduced pressure and the crude was triturated with 50% diethyl ether in pentane. It was then lyophilized to afford the compound Example 159 was obtained as white solid (20 mg, 25% yield, 98.14% purity). LCMS: m/z found 400.2 [M−H]⁺, rt=2.80 min (Method 2) [Waters Acquity BEH C8 column (1.7 μm, 50×2.1 mm)]; ¹H NMR (400 MHz, DMSO-d₆): δ 13.96 (s, 1H), 8.25 (s, 1H), 8.13 (s, 1H), 7.98 (d, J=8.5 Hz, 1H), 7.59 (d, J=8.2 Hz, 1H), 7.44-7.41 (m, 2H), 7.18 (t, J=8.7 Hz, 2H), 6.09-6.06 (m, 1H), 3.35-3.31 (m, 2H), 0.90 (t, J=8.6 Hz, 3H).

### Example 160 and Example 161

### (R)—N-ethyl-1-methyl-N-(2,2,2-trifluoro-1-(4-fluorophenyl)ethyl)-1H-indazole-6-sulfonamide (Example 160) and (R)—N-methyl-1-methyl-N-(2,2,2-trifluoro-1-(4-fluorophenyl)ethyl)-1H-indazole-6-sulfonamide (Example 161)

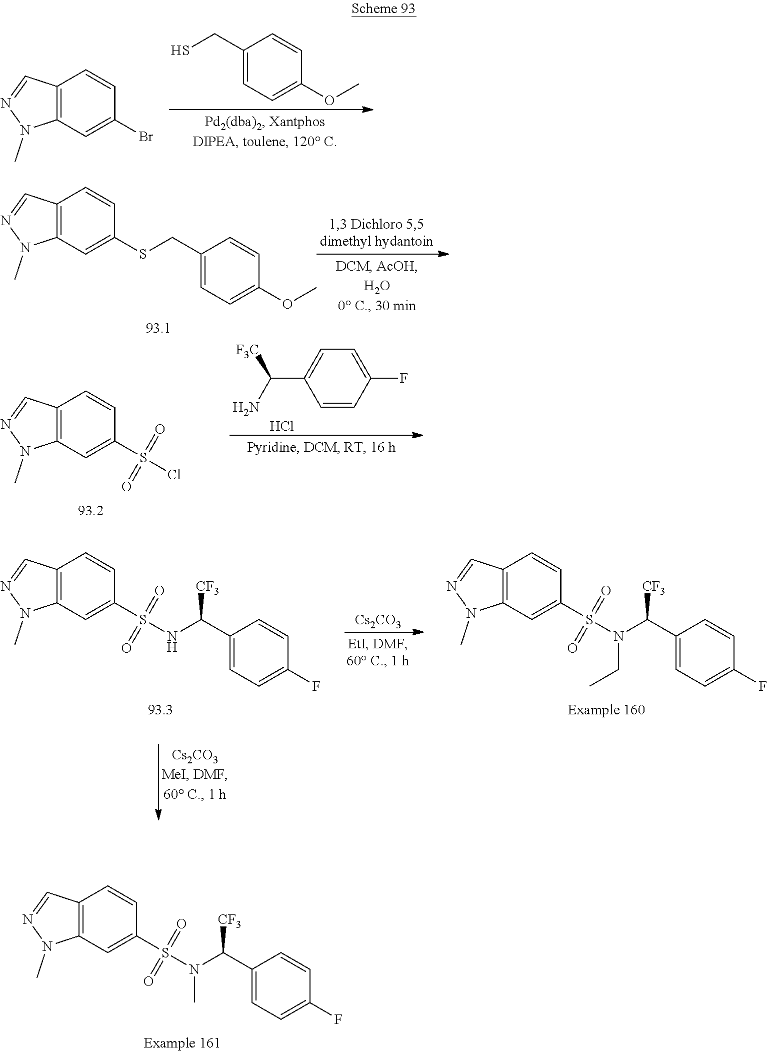

Synthesis of 6-((4-methoxybenzyl)thio)-1-methyl-1H-indazole 93.1: Intermediate 93.1 was synthesized from 6-bromo-1-methyl-1H-indazole following a method analogous to that described in Method AA using (4-methoxyphenyl)methanethiol. The crude was purified by column chromatography over silica gel using 20% ethyl acetate in hexane to provide the compound 93.1 as a yellow gum. (300 mg, 89% yield). ¹H NMR (400 MHz, DMSO-d₆): δ 7.96 (s, 1H), 7.64 (d, J=8.3 Hz, 1H), 7.59 (s, 1H), 7.30 (d, J=8.3 Hz, 2H), 7.06 (d, J=8.2 Hz, 2H), 6.85 (d, J=8.4 Hz, 1H), 4.28 (s, 2H), 3.99 (s, 3H), 3.71 (s, 3H).

Synthesis 1-methyl-1H-indazole-6-sulfonyl chloride 93.2: Sulfonyl chloride 93.2 was synthesized from intermediate 93.1 following the procedure described in Method AB. The crude sulfonyl chloride was used immediately in the following step without further purification.

Synthesis of (R)-1-methyl-N-(2,2,2-trifluoro-1-(4-fluorophenyl)ethyl)-1H-indazole-6-sulfonamide 93.3: intermediate 93.3 was synthesized from sulfonyl chloride 93.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to yield the compound 93.3 as a yellow gum (35 mg, 19%). ¹H NMR (400 MHz, DMSO-d₆): δ 9.28 (d, J=10.5 Hz, 1H), 8.09 (s, 1H), 7.97 (s, 1H), 7.73 (d, J=8.6 Hz, 1H), 7.40-7.30 (m, 3H), 6.91 (t, J=8.2 Hz, 2H), 5.40-5.35 (m, 1H), 4.05 (s, 3H).

(R)—N-ethyl-1-methyl-N-(2,2,2-trifluoro-1-(4-fluorophenyl)ethyl)-1H-indazole-6-sulfonamide (Example 160): The ethylation of intermediate 93.3 was preformed following the protocol as described in Method AD. The crude was purified by column chromatography over silica gel using 5:1 ethyl acetate:hexane to get the compound as a white solid 12 mg, 25% yield, 98.77% purity). LCMS: m/z found 416.2 [M+H]⁺, rt=3.16 min (Method 2) [Waters Acquity BEH C8 column (1.7 μm, 50×2.1 mm)]; ¹H NMR (400 MHz, DMSO-d₆): δ 8.32 (s, 1H), 8.21 (s, 1H), 7.94 (d, J=8.3 Hz, 1H), 7.57 (d, J=8.4 Hz, 1H), 7.42 (t, J=5.2 Hz, 2H), 7.17 (t, J=8.7 Hz, 2H), 6.07-5.99 (m, 1H), 4.16 (s, 3H), 3.42-3.31 (m, 1H), 3.25-3.15 (m, 1H), 0.96 (t, J=6.8 Hz, 3H).

(R)—N,1-dimethyl-N-(2,2,2-trifluoro-1-(4-fluorophenyl)ethyl)-1H-indazole-6-sulfonamide (Example 161): The methylation of intermediate 24.3 was performed following the protocol as described in Method AD. The crude was purified by column chromatography over silica gel using 5:1 ethyl acetate-hexane to obtain Example 161 as white solid (50 mg, 45% yield, 99.20% purity). LCMS: m/z found 402.19 [M+H]⁺, rt=3.15 min (Method 2) [Waters Acquity BEH C8 column (1.7 μm, 50×2.1 mm)]; ¹H NMR (400 MHz, DMSO-d₆): δ 8.31 (s, 1H), 8.21 (s, 1H), 7.94 (d, J=8.5 Hz, 1H), 7.52 (dd, J=1.3 Hz, 8.4 Hz, 1H), 7.43-7.37 (m, 2H), 7.14 (t, J=8.8 Hz, 2H), 6.07-5.99 (m, 1H), 4.16 (s, 3H), 2.74 (s, 3H).

### Example 162 and Example 163

### (R)—N-ethyl-2,2-difluoro-N-(2,2,2-trifluoro-1-(4-fluorophenyl)ethyl)benzo[d][1,3]dioxole-5-sulfonamide (Example 162) and (R)—N-methyl-2,2-difluoro-N-(2,2,2-trifluoro-1-(4-fluorophenyl)ethyl)benzo[d][1,3]dioxole-5-sulfonamide (Example 163)

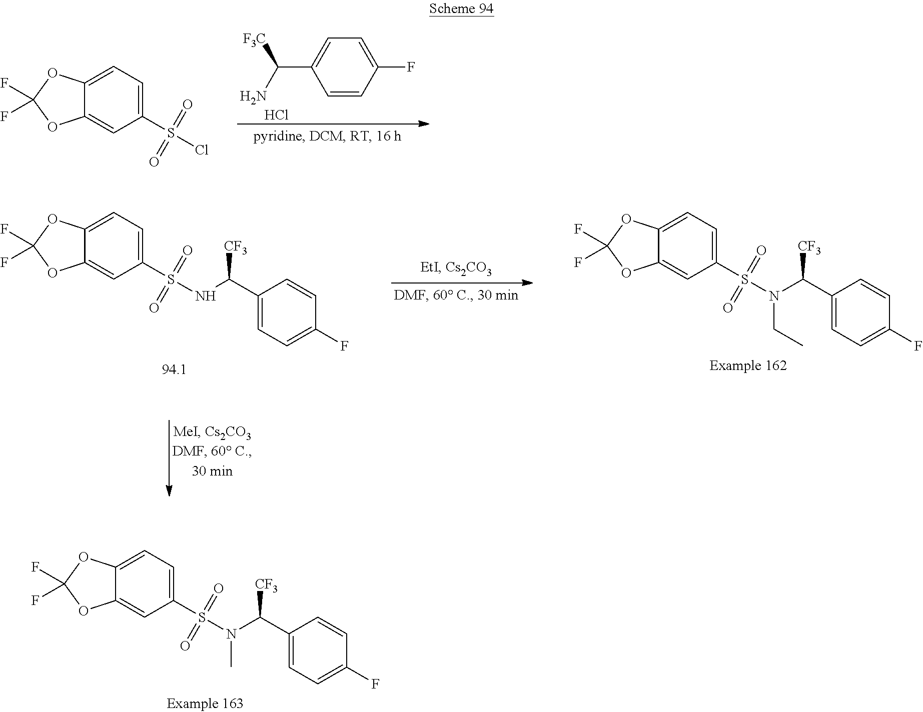 [SMILES: O=S(=O)(Cl)c1ccc2c(c1)OC(F)(F)O2 ; N[C@H](c1ccc(F)cc1)C(F)(F)F ; Cl ; O=S(=O)(N[C@H](c1ccc(F)cc1)C(F)(F)F)c1ccc2c(c1)OC(F)(F)O2 ; CCN([C@H](c1ccc(F)cc1)C(F)(F)F)S(=O)(=O)c1ccc2c(c1)OC(F)(F)O2 ; CN([C@H](c1ccc(F)cc1)C(F)(F)F)S(=O)(=O)c1ccc2c(c1)OC(F)(F)O2]

Synthesis of (R)-2,2-difluoro-N-(2,2,2-trifluoro-1-(4-fluorophenyl)ethyl)benzo[d][1,3]dioxole-5-sulfonamide 94.1: Sulfonamide 94.1 was synthesized from 2,2-difluorobenzo[d][1,3]dioxole-5-sulfonyl chloride following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to isolate 94.1 as a colorless oil (171 mg, 53% yield). LCMS: m/z found 412.0 [M−H], rt=1.99 min (Method 8) [Waters Xbridge C18 column (3.5 μm, 50×3 mm)].

Synthesis of (R)—N-ethyl-2,2-difluoro-N-(2,2,2-trifluoro-1-(4-fluorophenyl)ethyl)benzo[d][1,3]dioxole-5-sulfonamide: The ethylation of intermediate 94.1 was performed following the protocol as described in Method AD. The crude residue was purified by column chromatography over silica gel using 1:4 ethyl acetate-hexane to obtain compound Example 162 as colorless oil (65 mg, 40% yield, 98.94% purity). GC-MS (CHE 300-METHOD) (HP-5MS (30×250 μm×0.25 μm) m/z found 341.1. ¹H NMR (400 MHz, DMSO-d₆): δ 8.00 (d, J=2.0 Hz, 1H), 7.84 (d, J=8.4 Hz, 1H), 7.62 (d, J=8.4 Hz, 1H), 7.48-7.39 (m, 2H), 7.23 (t, J=8.4 Hz, 2H), 5.99 (q, J=8.7 Hz, 1H), 3.32-3.27 (m, 2H), 1.03-0.92 (m, 3H).

Synthesis of (R)-2,2-difluoro-N-methyl-N-(2,2,2-trifluoro-1-(4-fluorophenyl)ethyl)benzo[d][1,3]dioxole-5-sulfonamide: The methylation of intermediate 25.1 was preformed following the protocol as described in Method AD. The crude was purified by column chromatography over silica gel using 1:4 ethyl acetate-hexane to obtain the compound Example 163 as a colorless oil (88 mg, 48% yield, 96.70% purity). GC-MS (CHE 300-METHOD) (HP-5MS (30×250 μm×0.25 μm) m/z found 426.9. ¹H NMR (400 MHz, DMSO-d₆): δ 7.99 (d, J=1.8 Hz, 1H), 7.81 (dd, J=1.8, 8.4 Hz, 1H), 7.65 (d, J=8.5 Hz, 1H), 7.42 (dd, J=5.3, 8.5 Hz, 2H), 7.22 (t, J=8.8 Hz, 2H), 6.00 (q, J=8.7 Hz, 1H), 2.76 (s, 3H).

### Example 164

### (R)—N-ethyl-1-methyl-N-(2,2,2-trifluoro-1-(4-fluorophenyl)ethyl)-1H-benzo[d]imidazole-6-sulfonamide

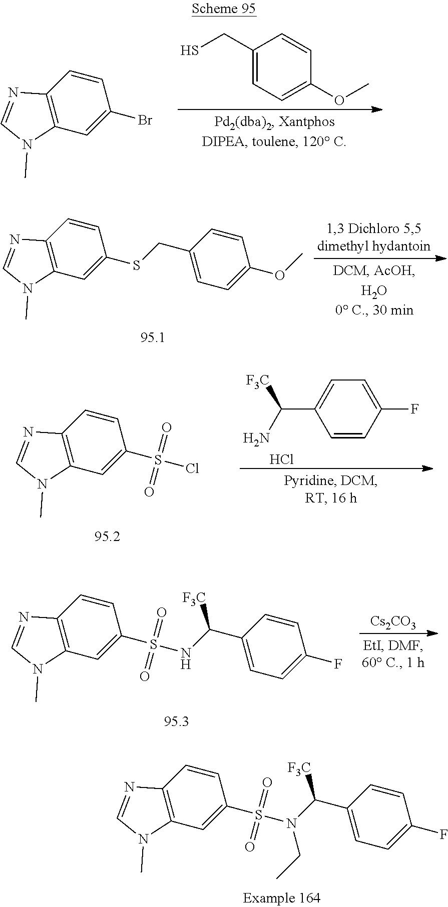

Synthesis of 6-((4-methoxybenzyl)thio)-1-methyl-1H-benzo[d]imidazole 95.1: Intermediate 95.1 was synthesized from 6-bromo-1-methyl-1H-benzo[d]imidazole following a method analogous to that described in Method AA using (4-methoxyphenyl)methanethiol. The crude was purified by column chromatography over silica gel using 20% ethyl acetate-hexane to get the compound 95.1 as a yellow gum (600 mg, 89% yield). ¹H NMR (400 MHz, DMSO-d₆): δ 8.14 (s, 1H), 7.60-7.50 (m, 2H), 7.27-7.15 (m, 3H), 6.83 (d, J=8.3 Hz, 2H), 4.19 (s, 2H), 3.79 (s, 3H), 3.70 (s, 3H).

Synthesis of 1-methyl-1H-benzo[d]imidazole-6-sulfonyl chloride 95.2: Sulfonyl chloride 95.2 was synthesized from intermediate 95.1 following the procedure described in Method AB. The crude sulfonyl chloride was used immediately in the forwarding step without further purification.

(R)-1-methyl-N-(2,2,2-trifluoro-1-(4-fluorophenyl)ethyl)-1H-benzo[d]imidazole-6-sulfonamide 95.3: Intermediate 95.3 was synthesized from sulfonyl chloride 95.2 following the protocol as described in Method AC using (R)-2,2,2-trifluoro-1-(4-fluorophenyl)ethan-1-amine hydrochloride. The compound was purified by column chromatography over silica gel using 3:7 ethyl acetate:hexane to get the compound 95.3 as a yellow gum (120 mg, 24%). ¹H NMR (400 MHz, DMSO-d₆): δ 9.20-9.10 (brs, 1H), 8.33 (s, 1H), 7.86 (s, 1H), 7.58 (d, J=8.8 Hz, 1H), 7.45 (d, J=8.4 Hz, 1H), 7.43-7.33 (m, 2H), 6.91 (d, J=8.4 Hz, 2H), 5.40-5.20 (m, 1H), 3.82 (s, 3H).

(R)—N-ethyl-1-methyl-N-(2,2,2-trifluoro-1-(4-fluorophenyl)ethyl)-1H-benzo[d]imidazole-6-sulfonamide: The ethylation of intermediate 26.3 was preformed following the protocol as described in Method AD. The crude was purified by column chromatography over silica gel using 5:1 ethyl acetate-hexane to obtained the compound Example 164 as a white solid (20 mg, 31% yield, 99.65% purity). LCMS: m/z found 416.22 [M+H]⁺, rt=2.98 min (Method 2) [Waters Acquity BEH C8 column (1.7 μm, 50×2.1 mm)]; ¹H NMR (400 MHz, DMSO-d₆): δ 8.45 (s, 1H), 8.23 (s, 1H), 7.80 (d, J=8.2 Hz, 1H), 7.71 (d, J=8.2 Hz, 1H), 7.49-7.36 (m, 2H), 7.17 (t, J=7.6 Hz, 2H), 6.08-5.97 (m, 1H), 3.92 (s, 3H), 3.21-3.08 (m, 1H), 0.93 (t, J=6.7 Hz, 3H).

### Example 165

### (R)—N-ethyl-N-(2,2,2-trifluoro-1-(4-fluorophenyl)ethyl)pyrazolo[1,5-a]pyrimidine-6-sulfonamide

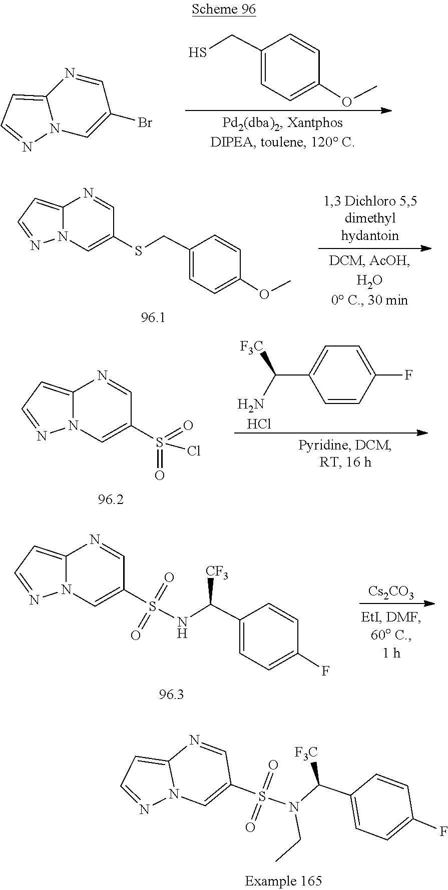

Synthesis of 6-((4-methoxybenzyl)thio)pyrazolo[1,5-a]pyrimidine 96.1 Intermediate 96.1 was synthesized from 6-bromopyrazolo[1,5-a]pyrimidine following a Buchwald protocol as described in Method AA using (4-methoxyphenyl)methanethiol. The crude was purified by column chromatography over silica gel using 50% ethyl acetate in hexane to obtain desired compound 96.1 as a yellow sticky gum (84% yield). LCMS: m/z found 272.34 [M+H+], rt=2.48 min (Method 9) [Waters Xbridge C18 column (3.5 μm, 50×3 mm)].

Synthesis of pyrazolo[1,5-a]pyrimidine-6-sulfonyl chloride 96.2: Intermediate 96.2 was synthesized following the protocol as described in Method AB. The crude sulfonyl chloride 96.2 was used in the next sulfonamidation step without further purification.

Synthesis of (R)—N-(2,2,2-trifluoro-1-(4-fluorophenyl)ethyl)pyrazolo[1,5-a]pyrimidine-6-sulfonamide 96.3: Intermediate 6.3 was synthesized following the protocol as described in Method AC using (R)-2,2,2-trifluoro-1-(4-fluorophenyl)ethan-1-amine hydrochloride. The crude was purified by column chromatography over silica gel using 30% ethyl acetate in hexane to obtain desired sulfonamide 96.3 as colourless sticky gum. LCMS: m/z found 375.1 [M+H]⁺, rt=3.28 min (Method 14) [Waters Acquity BEH C18 column (1.7 μm, 50×2.1 mm)].

Synthesis of (R)—N-ethyl-N-(2,2,2-trifluoro-1-(4-fluorophenyl)ethyl)pyrazolo[1,5-a]pyrimidine-6-sulfonamide: The ethylation of intermediate 27.3 was performed following the protocol as described in Method AD. The crude was purified by column chromatography over silica gel using 30% ethyl acetate in hexane to obtain the desired compound Example 165 (75 mg, 62% yield, 95.5% Purity) as white solid. LCMS: m/z found 403.19 [M+H], rt=3.03 min (Method 4) [Waters Xbridge C18 column (5 μm, 50×4.6 mm)]¹H NMR (400 MHz, DMSO-d₆): δ 9.74 (dd, J=0.9, 2.3 Hz, 1H), 8.87 (d, J=2.3 Hz, 1H), 8.51 (d, J=2.3 Hz, 1H), 7.53 (dd, J=5.4, 8.7 Hz, 2H), 7.22 (t, J=8.8 Hz, 2H), 6.96 (dd, J=0.9, 2.4 Hz, 1H), 6.14 (q, J=8.7 Hz, 1H), 3.57-3.33 (m, 2H), 1.02 (t, J=7.0 Hz, 3H).

### Example 166

### (R)—N-ethyl-N-(2,2,2-trifluoro-1-(4-fluorophenyl)ethyl)-1H-[1,2,3]triazolo[4,5-b]pyridine-6-sulfonamide

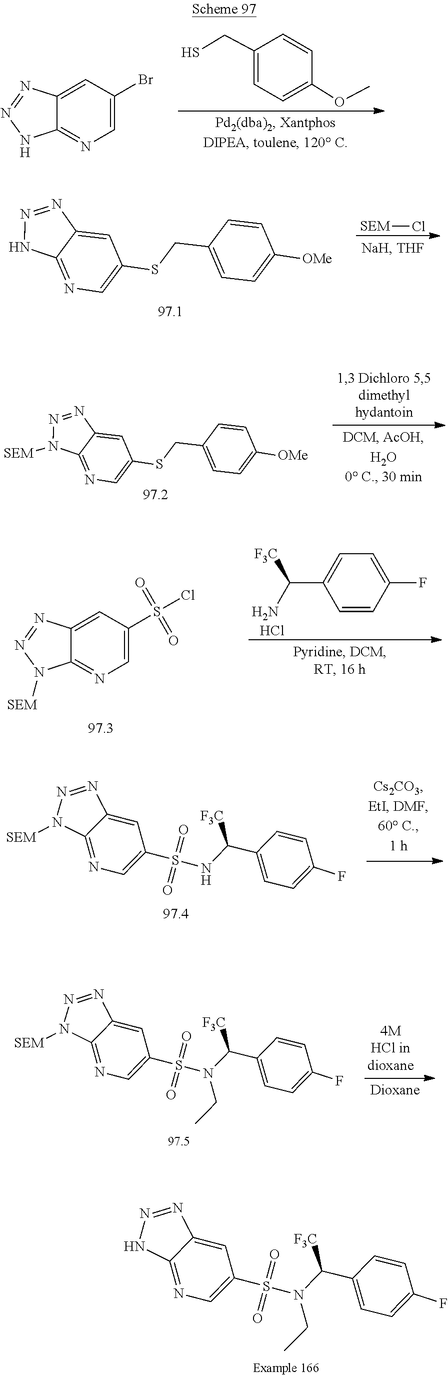

Synthesis of 6-((4-methoxybenzyl)thio)-3H-[1,2,3]triazolo[4,5-b]pyridine 97.1: Intermediate 97.1 was synthesized from 6-bromo-3H-[1,2,3]triazolo[4,5-b]pyridine following a Buchwald protocol as described in Method AA using (4-methoxyphenyl)methanethiol. The crude was purified by column chromatography over silica gel using 15% ethyl acetate-hexane mixture as eluent to afford desire product 97.1 (1.0 g, 73% yield). LCMS: m/z found 273.0 [M+H]⁺, rt=1.67 min (Method 3) [YMC Triart C18 column (3 μm, 33×2.1 mm)].

Synthesis of 6-((4-methoxybenzyl)thio)-3-((2-(trimethylsilyl)ethoxy)methyl)-3H-[1,2,3]triazolo[4,5-b]pyridine 97.2: N-SEM protection: Method AJ: A solution of intermediate 97.1 (1. g, 3.67 mmol) in THF (8 mL) was cooled at 0° C. and NaH (180 mg, 7.35 mmol) was added portion wise. It was stirred for 30 min and to it was added SEM-Cl (0.78 mL, 4.41 mmol). The reaction mixture was stirred for 2 h and was quenched with saturated NH₄Cl solution. It was extracted with ethyl acetate (2×30 mL), the combined organic layer was washed with water (25 mL) and brine (25 mL), dried over anhydrous Na₂SO₄ and concentrated under reduced pressure. The crude was further purified by column chromatography using 10% ethyl acetate in hexane as eluent to afford desire product 97.2 (600 mg, 40% yield). LCMS: m/z found 403.05 [M+H]⁺, rt=2.34 min (Method 3) [YMC Triart C18 column (3 μm, 33×2.1 mm)].

Synthesis of 3-((2-(trimethylsilyl)ethoxy)methyl)-3H-[1,2,3]triazolo[4,5-b]pyridine-6-sulfonyl chloride 97.3: Intermediate 97.3 was synthesized following the protocol as described in Method AB. The crude sulfonyl chloride 97.3 was used in the next sulfonamidation step without further purification.

Synthesis of (R)—N-(2,2,2-trifluoro-1-(4-fluorophenyl)ethyl)-3-((2-(trimethylsilyl)ethoxy)methyl)-3H-[1,2,3]triazolo[4,5-b]pyridine-6-sulfonamide 97.4: Intermediate 97.4 was synthesized following the protocol as described in Method AC using (R)-2,2,2-trifluoro-1-(4-fluorophenyl)ethan-1-amine hydrochloride. The crude was purified by column chromatography over silica gel using 20% ethyl acetate in hexane to obtain desired sulfonamide 97.4 as yellow gum (120 mg, 20% yield). LCMS: m/z found 503.1 [M+H]⁺, rt=2.03 min (Method 3) [YMC Triart C18 column (3 μm, 33×2.1 mm)].

Synthesis of (R)—N-ethyl-N-(2,2,2-trifluoro-1-(4-fluorophenyl)ethyl)-3-((2-(trimethylsilyl)ethoxy)methyl)-3H-[1,2,3]triazolo[4,5-b]pyridine-6-sulfonamide 97.5: The ethylation of intermediate 97.4 was performed following the protocol as described in Method AD. The crude was purified by column chromatography over silica gel using 30% ethyl acetate in hexane to obtain the desired compound 97.5 as colourless gum (50 mg, 39% yield). LCMS: m/z found 534.2 [M+H]⁺, rt=2.23 min (Method 3) [YMC Triart C18 column (3 μm, 33×2.1 mm)].

Synthesis of (R)—N-ethyl-N-(2,2,2-trifluoro-1-(4-fluorophenyl)ethyl)-3H-[1,2,3]triazolo[4,5-b]pyridine-6-sulfonamide: N-SEM deprotection: Method AK: To a stirred solution of intermediate 9.5 (50 mg, 0.09 mmol) in dioxane (1 mL) was added 4M HCl in dioxane (1 mL) at 5° C. The reaction mixture was stirred for 16 h at room temperature. The volatiles were evaporated under reduced pressure and the crude was purified by Reverse Phase Prep-HPLC to afford the desire product Example 166 as a white solid (20 mg, 53% yield, 98.41% purity) after lyophilization. RP method: Preparative HPLC was performed on a WATERS BGM 2545 EQUIPPED with WATERS PDA detector 2998 set to multiple-wavelength UV (200-400 nm) detection. Column name: YMC Actus Triart C18 (250×20 mm, 5μ) operating at ambient temperature and flow rate of 16 mL/min. Mobile phase: A=20 mM ammonium bicarbonate in water, B=Acetonitrile. Gradient profile: Mobile phase initial composition of 90% A and 10% B, then to 80% A and 20% B in 3 min, then to 40% A and 60% B in 18 min, then to 0% A and 100% B in 19 min, held in this composition up to 22 min for column washing, then returned to initial composition in 23 min an held till 27 min. LCMS: m/z found 404.17 [M+H]⁺, rt=3.00 min (Method B) [Waters Acquity BEH C8 column (1.7 μm, 50×2.1 mm)]. ¹H NMR (400 MHz, Chloroform-d): δ 9.18 (d, J=1.6 Hz, 1H), 8.93 (s, 1H), 7.51 (dd, J=4.9, 8.7 Hz, 2H), 7.11 (t, J=8.4 Hz, 2H), 5.88 (q, J=8.5 Hz, 1H), 3.47-3.35 (m, 1H), 3.30-3.16 (m, 1H), 0.94 (t, J=7.0 Hz, 3H).

### Example 167

### (R)—N-ethyl-N-(2,2,2-trifluoro-1-(4-fluorophenyl)ethyl)-1H-pyrazolo[3,4-c]pyridine-5-sulfonamide

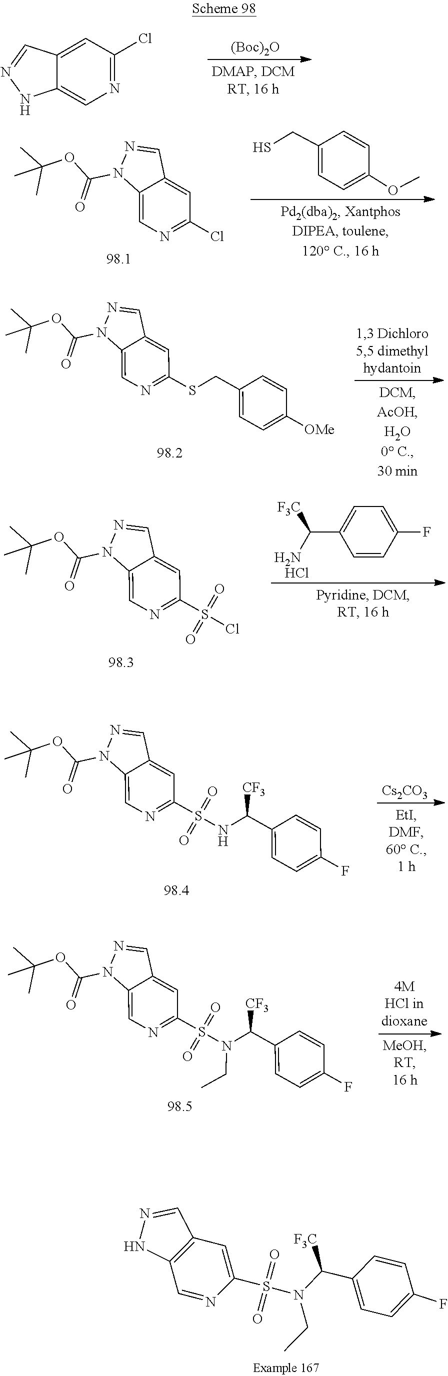

Synthesis of tert-butyl 5-chloro-1H-pyrazolo[3,4-c]pyridine-1-carboxylate 98.1. Intermediate 98.1 was synthesized from 5-chloro-1H-pyrazolo[3,4-c]pyridine following a method analogous to that described in Method AF. The crude was purified by column chromatography over silica gel using 20% ethyl acetate-hexane to get the compound 98.1 as a yellow gum (1.0 g, 60% yield). LCMS: m/z found 254.3 [M+H]⁺, rt=1.84 min (Method 2) [Waters Acquity BEH C8 column (1.7 μm, 50×2.1 mm)].

Synthesis of tert-butyl 5-((4-methoxybenzyl)thio)-1H-pyrazolo[3,4-c]pyridine-1-carboxylate 98.2: Intermediate 98.2 was synthesized from intermediate 98.1 following a method analogous to that described in Method AA using (4-methoxyphenyl)methanethiol. The crude residue was purified by column chromatography over silica gel using 20% ethyl acetate-hexane to afford the compound 98.2 as yellow gum (1.20 g, 64% yield).

Synthesis of tert-butyl 6-(chlorosulfonyl)-1H-pyrazolo[4,3-c]pyridine-1-carboxylate 98.3: Sulfonyl chloride 98.3 was synthesized from intermediate 98.2 following the procedure described in Method AB. The crude sulfonyl chloride was used immediately in the forwarding step without further purification.

Synthesis of tert-butyl (R)-5-(N-(2,2,2-trifluoro-1-(4-fluorophenyl)ethyl)sulfamoyl)-1H-pyrazolo[3,4-c]pyridine-1-carboxylate 98.4: Intermediate 98.4 was synthesized from sulfonyl chloride 98.3 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate compound 98.3 as yellow sticky gum (150 mg, 25% yield). LCMS: m/z found 475.1 [M+H]⁺, rt=1.92 min (Method 9) [Waters Xbridge C18 column (3.5 μm, 50×3 mm)].

Synthesis of tert-butyl (R)-5-(N-ethyl-N-(2,2,2-trifluoro-1-(4-fluorophenyl)ethyl)sulfamoyl)-1H-pyrazolo[3,4-c]pyridine-1-carboxylate 98.5: The ethylation of intermediate 98.4 was preformed following the protocol as described in Method AD. The crude was purified by column chromatography over silica gel using 5:1 ethyl acetate-hexane to provide the compound 98.5 as colorless gum (68 mg, 35% yield). ¹H NMR (400 MHz, DMSO-d₆): δ 9.39 (s, 1H), 8.85-8.69 (m, 2H), 7.51-7.18 (m, 4H), 5.94 (d, J=4.2 Hz, 1H), 3.37-3.28 (m, 2H), 1.69 (s, 9H), 0.91 (t, J=6.8 Hz, 3H).

Synthesis of (R)—N-ethyl-N-(2,2,2-trifluoro-1-(4-fluorophenyl)ethyl)-1H-pyrazolo[3,4-c]pyridine-5-sulfonamide: N-Boc deprotection of intermediate 98.4 was performed following a method analogous to that described in Method AG. The volatiles were evaporated under reduced pressure and the crude was triturated with 50% diethyl ether-pentane. The product was then lyophilized to obtain the compound Example 167 as a white solid (30 mg, 47% yield, 95.50% purity). LCMS: m/z found 403.2 [M+H]⁺, rt=2.72 min (Method 2) [Waters Acquity BEH C8 column (1.7 μm, 50×2.1 mm)]. ¹H NMR (400 MHz, DMSO-d₆): δ 14.17 (bs, 1H), 9.13 (s, 1H), 8.50-8.49 (m, 2H), 7.50 (t, J=6.4 Hz, 2H), 7.17 (t, J=8.8 Hz, 2H), 5.93-5.87 (m, 1H), 3.39-3.25 (m, 2H), 0.89 (t, J=6.8 Hz, 3H).

### Example 168

### (R)—N-ethyl-3-methyl-N-(2,2,2-trifluoro-1-(4-fluorophenyl)ethyl)-[1,2,4]triazolo[4,3-b]pyridazine-6-sulfonamide

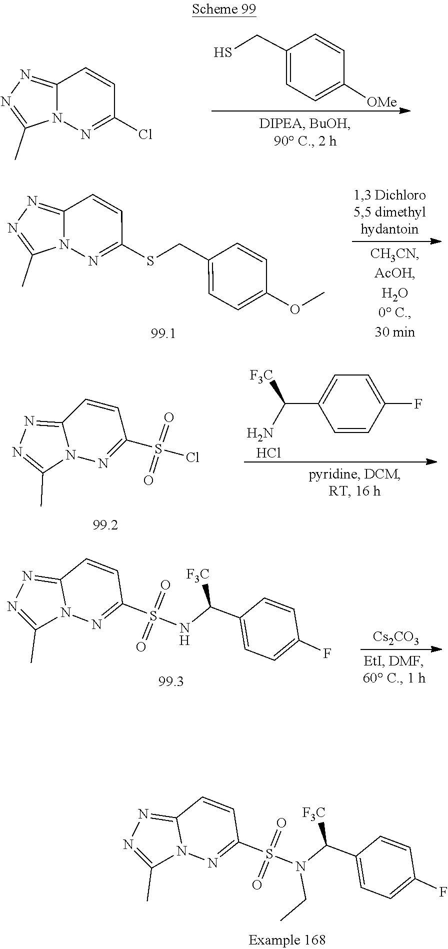

Synthesis of 6-((4-methoxybenzyl)thio)-3-methyl-[1,2,4]triazolo[4,3-b]pyridazine: SNAr reaction: Method AL: To stirred a solution of 6-chloro-3-methyl-[1,2,4]triazolo[4,3-b]pyridazine (2.0 g, 11.87 mmol) in n-BuOH (20 mL) 4-methoxyphenyl)methanethiol (1.48 mL, 10.68 mmol) and DIPEA (4.12 mL, 23.73 mmol) were added. The reaction mixture was heated at 90° C. for 2 h. The reaction mixture was diluted with water (20 mL) and extracted with ethyl acetate (2×50 mL). The combined organic layers were washed with water (25 mL) and brine (25 mL), dried over anhydrous Na₂SO₄ and concentrated under reduced pressure. The crude was purified by flash column chromatography using 45% ethyl acetate in hexane to afford desired intermediate 99.1 as a light brown sticky oil. LCMS: m/z found 287.0 [M+H]⁺, rt=1.69 min (Method 3) [YMC Triart C18 column (3 μm, 33×2.1 mm)].

Synthesis of 3-methyl-[1,2,4]triazolo[4,3-b]pyridazine-6-sulfonyl chloride 9.2: Sulfonyl chloride 30.2 was synthesized from intermediate 99.1 following the procedure described in Method AB. The crude sulfonyl chloride was used immediately in the forwarding step without further purification.

Synthesis of (R)-3-methyl-N-(2,2,2-trifluoro-1-(4-fluorophenyl)ethyl)-[1,2,4]triazolo[4,3-b]pyridazine-6-sulfonamide 99.3: Intermediate 99.3 was synthesized from sulfonyl chloride 9.2 following the protocol as described in Method AC using (R)-2,2,2-trifluoro-1-(4-fluorophenyl)ethan-1-amine hydrochloride. The compound was purified by column chromatography over silica gel using 70% ethyl acetate in hexane to isolate compound 99.3 as yellow sticky gum (150 mg, 25% yield). LCMS: m/z found 390.0 [M+H]⁺, rt=1.67 min (Method 3) [YMC Triart C18 column (3 μm, 33×2.1 mm)].

Synthesis of (R)—N-ethyl-3-methyl-N-(2,2,2-trifluoro-1-(4-fluorophenyl)ethyl)-[1,2,4]triazolo[4,3-b]pyridazine-6-sulfonamide: The ethylation of intermediate 99.3 was performed following the protocol as described in Method AD. The crude was purified by Reverse Phase Prep-HPLC to afford the desire product Example 168 was isolated as white solid (10 mg, 13% yield, 97.18% purity). RP method: Preparative HPLC was done in WATERS BGM 2545 EQUIPPED with PDA detector set to multiple-wavelength UV (200-400 nm) detection. Column name: XBridge C18 (50×19 mm, 5μ) operating at ambient temperature and flow rate of 24 mL/min. Mobile phase: A=20 mM Ammonium Bicarbonate in water, Mobile phase B=Acetonitrile. Gradient profile: Mobile phase initial composition of 90% A and 10% B, then to 10% A and 90% B in 8 min, held in this composition up to 10 min for column washing, then returned to initial composition in 10.5 min and held for 12 min. LCMS: m/z found 418.31 [M+H]⁺, rt=2.94 min (Method 2) [Waters Acquity BEH C8 column (1.7 μm, 50×2.1 mm)]. ¹H NMR (400 MHz, DMSO-d₆): δ 8.58 (d, J=9.7 Hz, 1H), 7.76 (d, J=9.7 Hz, 1H), 7.55 (dd, J=5.4, 8.7 Hz, 2H), 7.22 (t, J=8.8 Hz, 2H), 6.03 (q, J=8.7 Hz, 1H), 3.67-3.44 (m, 2H), 2.58 (s, 3H), 1.05 (t, J=7.0 Hz, 3H).

### Example 169 and Example 170

### (R)—N-ethyl-N-(2,2,2-trifluoro-1-(4-fluorophenyl)ethyl)-3-(trifluoromethyl)-[1,2,4]triazolo[4,3-b]pyridazine-6-sulfonamide (Example 169) and (R)—N-methyl-N-(2,2,2-trifluoro-1-(4-fluorophenyl)ethyl)-3-(trifluoromethyl)-[1,2,4]triazolo[4,3-b]pyridazine-6-sulfonamide (Example 170)

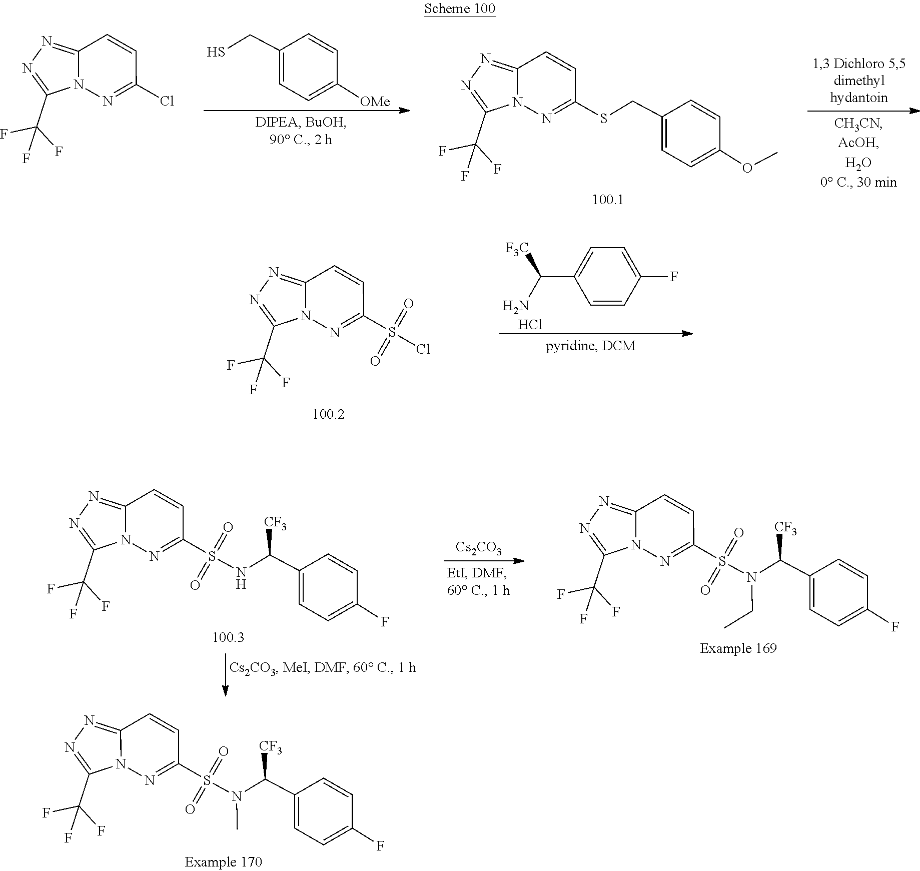

Synthesis of 6-((4-methoxybenzyl)thio)-3-(trifluoromethyl)-[1,2,4]triazolo[4,3-b]pyridazine 100.1: Intermediate 100.1 was synthesized from 6-chloro-3-(trifluoromethyl)-[1,2,4]triazolo[4,3-b]pyridazine following a method analogous to that described in Method AL using (4-methoxyphenyl)methanethiol. The crude was purified by column chromatography over silica gel using 20% ethyl acetate-hexane to get the compound 100.2 as a yellow gum (1.0 g, 65% yield). ¹H NMR (400 MHz, DMSO-d₆): δ 8.37 (t, J=9.7 Hz, 1H), 7.58-7.48 (m, 1H), 7.40 (d, J=8.5 Hz, 2H), 6.87 (d, J=8.5 Hz, 2H), 4.40 (s, 2H), 3.71 (s, 3H).

Synthesis of 3-(trifluoromethyl)-[1,2,4]triazolo[4,3-b]pyridazine-6-sulfonyl chloride 100.2: Sulfonyl chloride 100.2 was synthesized from intermediate 100.1 following the procedure described in Method AB. The crude sulfonyl chloride was used immediately in the forwarding step without further purification.

Synthesis of (R)—N-(2,2,2-trifluoro-1-(4-fluorophenyl)ethyl)-3-(trifluoromethyl)-[1,2,4]triazolo[4,3-b]pyridazine-6-sulfonamide 100.3: Intermediate 100.3 was synthesized from sulfonyl chloride 100.2 following the protocol as described in Method AC using (R)-2,2,2-trifluoro-1-(4-fluorophenyl)ethan-1-amine hydrochloride. The crude was triturated with hexane and used in the forwarding step without further purification.

Synthesis of (R)—N-ethyl-N-(2,2,2-trifluoro-1-(4-fluorophenyl)ethyl)-3-(trifluoromethyl)-[1,2,4]triazolo[4,3-b]pyridazine-6-sulfonamide: The ethylation of intermediate 100.3 was preformed following the protocol as described in Method AD. The crude was purified by Reverse Phase Prep-HPLC to afford the desire product Example 169 was isolated as white solid (50 mg, 33% yield, 99.09% purity). RP method: Preparative HPLC Was done in WATERS BGM 2545 EQUIPPED with WATERS PDA detector 2998 set to multiple-wavelength UV (200-400 nm) detection. Column name: YMC Actus Triart C18 (250×20 mm, 5μ) operating at ambient temperature and flow rate of 16 mL/min. Mobile phase: A=20 mM ammonium bicarbonate in water, B=Acetonitrile. Gradient profile: Mobile phase initial composition of 50% A and 50% B, then to 15.4% A and 84.6% B in 15.58 min, then to 0% A and 100% B in 15.65 min, held in this composition up to 17.65 min for column washing, then returned to initial composition in 17.70 min an held for 20.70 min. LCMS: m/z found 472.26 [M+H]⁺, rt=3.22 min (Method 2) [Waters Acquity BEH C8 column (1.7 μm, 50×2.1 mm)]; ¹H NMR (400 MHz, DMSO-d₆): δ 8.90 (d, J=9.8 Hz, 1H), 8.07 (d, J=9.8 Hz, 1H), 7.52 (dd, J=5.4, 8.5 Hz, 2H), 7.22 (t, J=8.7 Hz, 2H), 6.08 (q, J=8.0 Hz, 1H), 3.62-3.44 (m, 2H), 1.02 (t, J=7.1 Hz, 3H).

Synthesis of (R)—N-methyl-N-(2,2,2-trifluoro-1-(4-fluorophenyl)ethyl)-3-(trifluoromethyl)-[1,2,4]triazolo[4,3-b]pyridazine-6-sulfonamide: The methylation of intermediate 31.3 was preformed following the protocol as described in Method AD. The crude was purified by Reverse Phase Prep-HPLC to afford the desire product Example 170 was isolated as white solid (25 mg, 20% yield, 97.85% purity). RP method: Preparative HPLC Was done in WATERS BGM 2545 EQUIPPED with WATERS PDA detector 2998 set to multiple-wavelength UV (200-400 nm) detection. Column name: YMC Actus Triart C18 (250×20 mm, 5μ) operating at ambient temperature and flow rate of 16 mL/min. Mobile phase: A=20 mM ammonium bicarbonate in water, B=Acetonitrile. Gradient profile: Mobile phase initial composition of 50% A and 50% B, then to 20% A and 80% B in 18 min, then to 5% A and 95% B in 19 min, held in this composition up to 22 min for column washing, then returned to initial composition in 22.5 min an held for 25 min. LCMS: m/z found 458.23 [M+H]⁺, rt=3.16 min (Method 2) [Waters Acquity BEH C8 column (1.7 μm, 50×2.1 mm)]; ¹H NMR (400 MHz, DMSO-d₆): δ 8.91 (d, J=9.8 Hz, 1H), 8.07 (d, J=9.8 Hz, 1H), 7.49 (dd, J=5.3, 8.6 Hz, 2H), 7.22 (t, J=8.8 Hz, 2H), 6.11 (q, J=8.6 Hz, 1H), 2.95 (s, 3H).

### Example 171

### (R)—N-(1-(4-chlorophenyl)-2,2,2-trifluoroethyl)-5-cyano-N-methylpyridine-3-sulfonamide

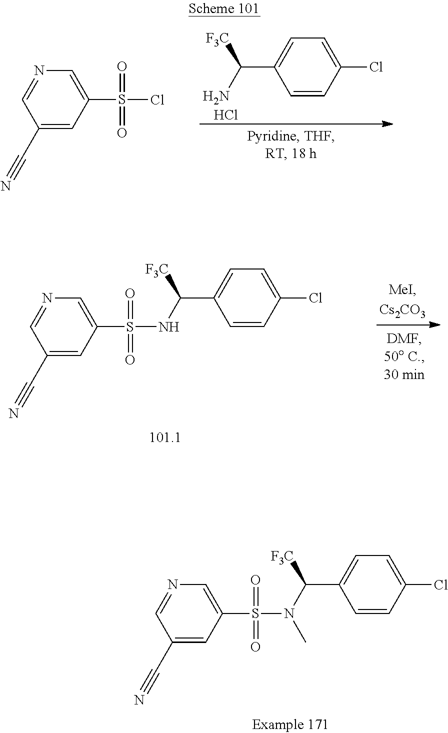 [SMILES: N#Cc1cncc(S(=O)(=O)Cl)c1 ; Cl ; N[C@H](c1ccc(Cl)cc1)C(F)(F)F ; N#Cc1cncc(S(=O)(=O)N[C@H](c2ccc(Cl)cc2)C(F)(F)F)c1 ; CN([C@H](c1ccc(Cl)cc1)C(F)(F)F)S(=O)(=O)c1cncc(C#N)c1]

Synthesis of (R)—N-(1-(4-chlorophenyl)-2,2,2-trifluoroethyl)-5-cyanopyridine-3-sulfonamide 101.1: Method O: 5-cyanopyridine-3-sulfonyl chloride (200 mg, 0.98 mmol) was dissolved in THF (1 mL) and (R)-1-(4-chlorophenyl)-2,2,2-trifluoroethan-1-amine (170 mg, 0.69 mmol) was added to it followed by pyridine (0.5 mL, 6.91 mmol). The reaction mixture was stirred at RT overnight. Then, it was diluted with water (5 mL) and extracted with ethyl acetate (2×20 mL). The combined organic layers were washed with water (15 mL) and brine (15 mL), dried and the solvent was evaporated under reduced pressure. The product was purified by column chromatography over silica gel and the compound 101.1 was isolated as a sticky gum (180 mg, 49% yield). ¹H NMR (400 MHz, DMSO-d₆) δ 9.89-9.82 (m, 1H), 9.15 (d, J=1.7 Hz, 1H), 9.02 (d, J=2.1 Hz, 1H), 8.44 (d, J=1.9 Hz, 1H), 7.41 (d, J=8.5 Hz, 2H), 7.36 (d, J=8.6 Hz, 2H), 5.54-5.50 (m, 1H).

Synthesis of (R)—N-(1-(4-chlorophenyl)-2,2,2-trifluoroethyl)-5-cyano-N-methylpyridine-3-sulfonamide: Alkylation (Method AD): The sulfonamide 101.1 (100 mg, 0.26 mmol) was dissolved in DMF (1 mL) in a sealed tube and Cs₂CO₃ (130 mg, 0.4 mmol) was added to it. The reaction mixture was stirred and methyl iodide (0.2 mL, 2.66 mmol) was added. Stirring was continued at 50° C. for 30 min. The reaction was then quenched with water (10 mL) and extracted with ethyl acetate (2×10 mL). The combined organic layers were washed with water (10 mL) and brine (10 mL), dried over anhydrous Na₂SO₄ and concentrated under reduced pressure. The product was purified by column chromatography over silica gel using 20% ethyl acetate in hexane to yield Example 171 as a colorless sticky gum (40 mg, 99.55% purity, 70% yield). LCMS: m/z found 390.11 [M+H]⁺, rt=3.52 min (Method 2) [Waters Acquity BEH C8 column (1.7 μm, 50×2.1 mm)]; ¹H NMR (400 MHz, DMSO-d₆) δ 9.32 (d, J=2.0 Hz, 2H), 8.89 (t, J=2.0 Hz, 1H), 7.53-7.45 (m, 2H), 7.39 (d, J=8.6 Hz, 2H), 6.09 (q, J=8.5 Hz, 1H), 2.86 (s, 3H).

### Example 172

### (R)—N-methyl-N-(2,2,2-trifluoro-1-(4-(trifluoromethyl)phenyl)ethyl)pyrimidine-5-sulfonamide

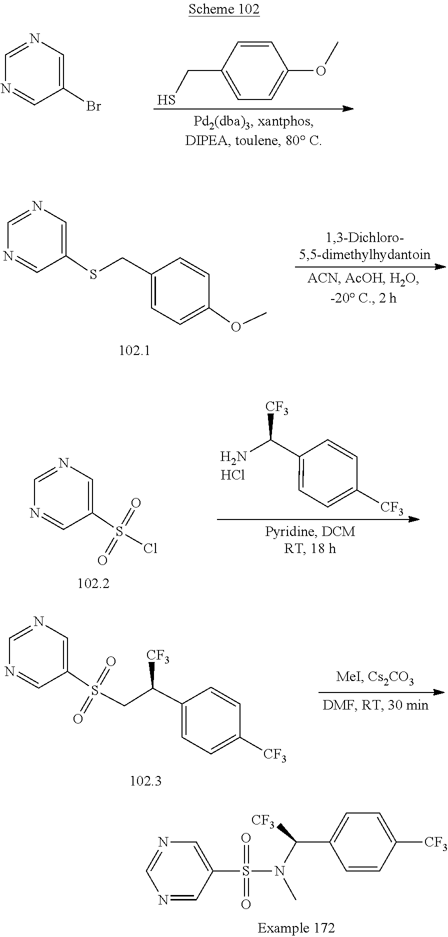

Synthesis of 5-((4-methoxybenzyl)thio)pyrimidine 102.1: Buchwald Coupling; Method AA: 5-bromopyrimidine (5.0 g, 31.45 mmol) was dissolved in toluene (30 mL) and to this solution (4-methoxyphenyl)methanethiol (3.9 mL, 28.30 mmol) was added. The solution was degasified with argon and DIPEA (11 mL, 62.90 mmol) was added to it. Xantphos (910 mg, 1.57 mmol) was added to it under the inert atmosphere, followed by Pd₂(dba)₃ (836 mg, 0.94 mmol). The reaction mixture was then stirred at 80° C. for 16 h under argon atmosphere. After completion (monitored by TLC and LCMS), it was diluted with ethyl acetate (50 mL) and filtered through sintered funnel. The filtrate was washed with water (50 mL) and brine (50 mL), dried over anhydrous Na₂SO₄ and the solvent was evaporated under reduced pressure. The product was purified by column chromatography over silica gel using 20% ethyl acetate in hexane to provide the product 102.1 as a yellow gum (2.5 g, 65% yield). LCMS: m/z found 233.3 [M+H]⁺, rt=1.81 min (Method 31) [Waters Acquity BEH C18 column (1.7 μm, 50×2.1 mm)].

Synthesis of pyrimidine-5-sulfonyl chloride 102.2: Synthesis of sulfonyl chloride; Method AB: A stirred solution of intermediate 102.1 (3.70 g, 15.95 mmol) in MeCN (40 mL) was cooled at −20° C. and to the solution a mixture of AcOH (2.5 mL) and H₂O (5 mL) was added. The solution was stirred and 1,3-Dichloro-5,5-dimethyl hydantoin (6.28 g, 31.90 mmol) was added portion wise to it. The stirring was continued at the same temperature for 2 h. It was then diluted with ice cold water (20 mL) and extracted with DCM (15 mL). The organic layers were washed with water (20 mL), dried over anhydrous Na₂SO₄. The reaction mixture was then filtered, and the organic part was directly used in the forwarding step without evaporation.

Synthesis of (R)—N-(2,2,2-trifluoro-1-(4-(trifluoromethyl)phenyl)ethyl)pyrimidine-5-sulfonamide 102.3: Method O: To the crude sulfonyl chloride 102.2 (R)-2,2,2-trifluoro-1-(4-(trifluoromethyl)phenyl)ethan-1-amine hydrochloride (1.8 g, 7.42 mmol) and pyridine (6.5 mL, 80.92 mmol) were added. The reaction mixture was stirred at room temperature for 16 h. The volatiles were then evaporated under reduced pressure and the crude was purified by column chromatography over silica gel using 3:7 ethyl acetate:hexane to provide compound 102.3 as a colorless gum (1.50 g, 58% yield). ¹H NMR (400 MHz, DMSO-d₆): δ 10.02 (s, 1H), 9.27 (s, 1H), 8.99 (s, 2H), 7.71-7.65 (m, 4H), 5.75-5.65 (m, 1H).

Synthesis of (R)—N-methyl-N-(2,2,2-trifluoro-1-(4-(trifluoromethyl)phenyl)ethyl)pyrimidine-5-sulfonamide: The methylation of intermediate 102.3 was performed following the protocol as described in Method AD. The product was purified by column chromatography over silica gel using 20% ethyl acetate-hexane to obtain compound Example 172 as a colorless sticky gum (210 mg, 99.07% purity, 23% yield). LCMS: m/z found 400.14 [M+H]⁺, rt=3.18 min (Method B) [Waters Acquity BEH C8 column (1.7 μm, 50×2.1 mm)]; ¹H NMR (400 MHz, Chloroform-d) δ 9.42 (s, 1H), 9.15 (s, 2H), 7.72 (d, J=8.3 Hz, 2H), 7.60 (d, J=8.1 Hz, 2H), 5.94 (q, J=8.2 Hz, 1H), 2.77 (s, 3H).

### Example 173 and Example 174

### (R)—N,1-diethyl-N-(2,2,2-trifluoro-1-(4-fluorophenyl)ethyl)-1H-pyrazolo[4,3-b]pyridine-3-sulfonamide (Example 173) and (R)—N-ethyl-N-(2,2,2-trifluoro-1-(4-fluorophenyl)ethyl)-1H-pyrazolo[4,3-b]pyridine-3-sulfonamide (Example 174)

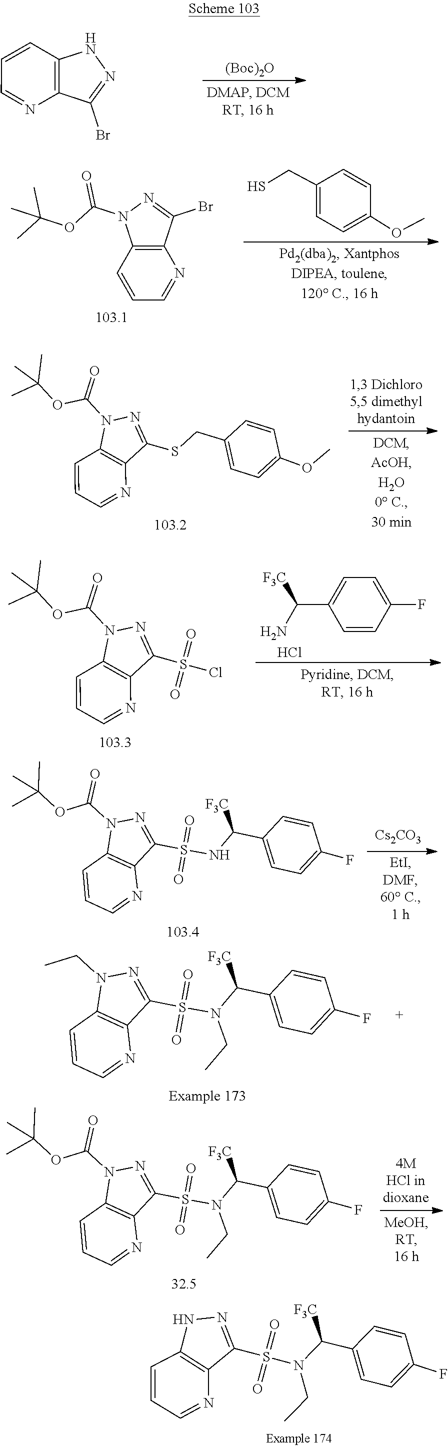

Synthesis of tert-butyl 3-bromo-1H-pyrazolo[4,3-b]pyridine-1-carboxylate 13.1: Intermediate 103.1 was synthesized from 3-chloro-1H-pyrazolo[4,3-b]pyridine following a method analogous to that described in Method AF. The crude residue was purified by column chromatography over silica gel using 20% ethyl acetate-hexane to provide the compound 103.1 as a yellow gum (550 mg, 72% yield). ¹H NMR (400 MHz, DMSO-d₆): δ 8.77 (d, J=4.4 Hz, 1H), 8.44 (d, J=8.5 Hz, 1H), 7.76-7.71 (m, 1H), 1.66 (s, 9H).

Synthesis of tert-butyl 3-((4-methoxybenzyl)thio)-1H-pyrazolo[4,3-b]pyridine-1-carboxylate 103.2: Intermediate 103.2 was synthesized from intermediate 103.1 following a method analogous to that described in Method AA using (4-methoxyphenyl)methanethiol. The crude residue was purified by column chromatography over silica gel using 20% ethyl acetate-hexane to get the compound 103.2 as yellow gum (330 mg, 48% yield). ¹H NMR (400 MHz, DMSO-d₆): δ 8.65 (d, J=3.8 Hz, 1H), 8.36 (d, J=8.4 Hz, 1H), 7.68-7.62 (m, 1H), 7.41 (d, J=8.5 Hz, 2H), 6.86 (d, J=8.5 Hz, 2H), 4.53 (s, 2H), 3.71 (s, 2H), 1.674 (s, 9H).

Synthesis of tert-butyl 3-(chlorosulfonyl)-1H-pyrazolo[4,3-b]pyridine-1-carboxylate 103.3: Sulfonyl chloride 103.3 was synthesized from intermediate 103.2 following the procedure described in Method AB. The crude sulfonyl chloride was used immediately in the forwarding step without further purification.

Synthesis of tert-butyl (R)-3-(N-(2,2,2-trifluoro-1-(4-fluorophenyl)ethyl)sulfamoyl)-1H-pyrazolo[4,3-b]pyridine-1-carboxylate 103.4: Intermediate 103.4 was synthesized from sulfonyl chloride 103.3 following the protocol as described in Method AC using (R)-2,2,2-trifluoro-1-(4-fluorophenyl)ethan-1-amine hydrochloride. The compound was purified by column chromatography over silica gel using 40% ethyl acetate in hexane to isolate compound 103.4 as a yellow sticky gum (130 mg, 37% yield). ¹H NMR (400 MHz, Chloroform-d): δ 8.73 (d, J=4.2 Hz, 1H), 8.41 (d, J=8.6 Hz, 1H), 7.52-7.48 (m, 1H), 7.20-7.10 (m, 2H), 6.80-6.70 (m, 2H), 5.30-5.20 (m, 1H), 1.72 (s, 9H).

Synthesis of tert-butyl (R)-3-(N-ethyl-N-(2,2,2-trifluoro-1-(4-fluorophenyl)ethyl)sulfamoyl)-1H-pyrazolo[4,3-b]pyridine-1-carboxylate 32.5 and (R)—N,1-diethyl-N-(2,2,2-trifluoro-1-(4-fluorophenyl)ethyl)-1H-pyrazolo[4,3-b]pyridine-3-sulfonamide: The ethylation of intermediate 103.4 was preformed following the protocol as described in Method AD. The crude was purified by column chromatography over silica gel using 5:1 ethyl acetate-hexane and the both the compounds 103.5 and Example 173 were isolated.

(R)-3-(N-ethyl-N-(2,2,2-trifluoro-1-(4-fluorophenyl)ethyl)sulfamoyl)-1H-pyrazolo[4,3-b]pyridine-1-carboxylate 103.5: Colourless gum (20 mg, 12% yield). ¹H NMR (400 MHz, Chloroform-d): δ 8.84-8.80 (m, 1H), 8.51-8.48 (m, 1H), 7.56-7.50 (m, 3H), 7.00 (t, J=8.5 Hz, 2H), 6.05-6.0 (m, 1H), 3.62-3.59 (m, 2H). 1.55 (s, 9H), 1.06 (t, J=7.0 Hz, 3H).

(R)—N,1-diethyl-N-(2,2,2-trifluoro-1-(4-fluorophenyl)ethyl)-1H-pyrazolo[4,3-b]pyridine-3-sulfonamide (Example 173): White solid (25 mg, 99.80% purity, 17% yield). LCMS: m/z found 431.2 [M+H]⁺, rt=3.14 (Method 2) [Waters Acquity BEH C8 column (1.7 μm, 50×2.1 mm)] min; ¹H NMR (400 MHz, Chloroform-d) δ 8.78 (d, J=4.0 Hz, 1H), 7.84 (d, J=8.4 Hz, 1H), 7.47 (t, J=8.0 Hz, 2H), 7.43-7.37 (m, 1H), 6.97 (t, J=8.4 Hz, 2H), 6.01 (q, J=8.3 Hz, 1H), 4.52 (q, J=7.2 Hz, 2H), 3.66-3.46 (m, 2H), 1.08 (t, J=7.0 Hz, 3H).

Synthesis of (R)—N-ethyl-N-(2,2,2-trifluoro-1-(4-fluorophenyl)ethyl)-1H-pyrazolo[4,3-b]pyridine-3-sulfonamide: N-Boc deprotection of intermediate 32.5 was performed following a method analogous to that described in Method AG. The volatiles were evaporated under reduced pressure and the crude was triturated with 50% diethyl ether-pentane. It was then lyophilized to afford the compound Example 174 as brown sticky solid (10 mg, 99.20% purity, 40% yield). LCMS: m/z found 403.29 [M+H]⁺, rt=2.95 min (Method 2) [Waters Acquity BEH C8 column (1.7 μm, 50×2.1 mm)]; ¹H NMR (400 MHz, DMSO-d₆): δ 14.38 (s, 1H), 8.69 (d, J=4.0 Hz, 1H), 8.17 (d, J=8.3 Hz, 1H), 7.52 (s, 1H), 7.33 (t, J=7.0 Hz, 2H), 7.09 (t, J=7.8 Hz, 2H), 5.98-5.88 (m, 1H), 3.97-3.49 (m, 1H), 0.99 (t, J=7.0 Hz, 3H).

### Example 175

### (R)—N-ethyl-N-(2,2,2-trifluoro-1-(4-fluorophenyl)ethyl)-1H-pyrrolo[2,3-b]pyridine-4-sulfonamide

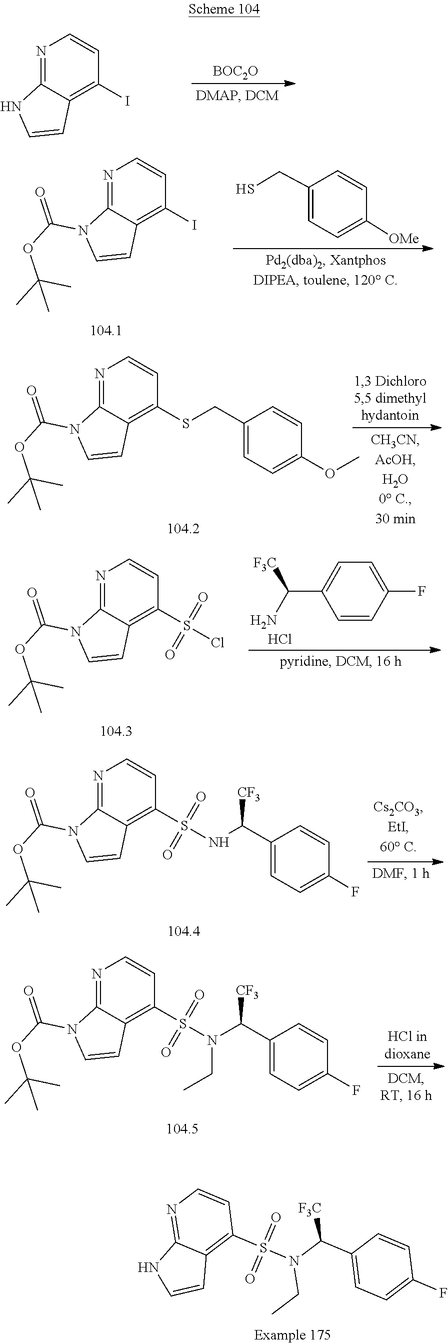

Synthesis of tert-butyl 4-iodo-1H-pyrrolo[2,3-b]pyridine-1-carboxylate 104.1: Intermediate 104.1 was synthesized from 4-iodo-1H-pyrrolo[2,3-b]pyridine following a method analogous to that described in Method AF. The crude was purified by column chromatography over silica gel using 20% ethyl acetate-hexane to obtain the compound 1.1 was isolated as a yellow oil (1.2 g, 83% yield). ¹H NMR (400 MHz, DMSO-d₆): δ 8.05 (d, J=5.0 Hz, 1H), 7.88 (d, J=4.1 Hz, 1H), 7.73 (d, J=5.0 Hz, 1H), 6.50 (d, J=4.1 Hz, 1H), 1.61 (s, 9H).

Synthesis of tert-butyl 4-((4-methoxybenzyl)thio)-1H-pyrrolo[2,3-b]pyridine-1-carboxylate 104.2: Intermediate 104.2 was synthesized from intermediate 104.1 following a method analogous to that described in Method A using (4-methoxyphenyl)methanethiol. The crude was purified by column chromatography over silica gel using 20% ethyl acetate-hexane to yield compound 104.2 as a yellow gum (1.0 g, 85% yield). ¹H NMR (400 MHz, DMSO-d₆): δ 8.24 (d, J=5.0 Hz, 1H), 7.73 (d, J=4.0 Hz, 1H), 7.37 (d, J=8.5 Hz, 2H), 7.28 (d, J=5.2 Hz, 1H), 6.88 (d, J=4.0 Hz, 2H), 6.60 (d, J=4.0 Hz, 1H), 4.40 (s, 2H), 3.72 (s, 3H), 1.59 (s, 9H).

Synthesis of tert-butyl 4-(chlorosulfonyl)-1H-pyrrolo[2,3-b]pyridine-1-carboxylate 104.3: Sulfonyl chloride 104.3 was synthesized from intermediate 104.2 following the procedure described in Method AB. The crude sulfonyl chloride was used immediately in the forwarding step without further purification.

Synthesis of tert-butyl (R)-4-(N-(2,2,2-trifluoro-1-(4-fluorophenyl)ethyl)sulfamoyl)-1H-pyrrolo[2,3-b]pyridine-1-carboxylate 104.4: Intermediate 104.4 was synthesized from sulfonyl chloride 104.3 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate compound 104.4 as a yellow sticky gum (300 mg, 31% yield). ¹H NMR (400 MHz, DMSO-d₆): δ 9.73 (d, J=10.3 Hz, 1H), 8.45 (d, J=4.9 Hz, 1H), 7.92 (d, J=3.9 Hz, 1H), 7.51 (d, J=5.0 Hz, 1H), 7.35-7.30 (m, 2H), 7.00 (d, J=3.9 Hz, 1H), 6.92 (t, J=7.2 Hz, 2H), 5.45-5.32 (m, 1H), 1.60 (s, 9H).

Synthesis of tert-butyl (R)-4-(N-ethyl-N-(2,2,2-trifluoro-1-(4-fluorophenyl)ethyl)sulfamoyl)-1H-pyrrolo[2,3-b]pyridine-1-carboxylate 104.5: The ethylation of intermediate 104.4 was preformed following the protocol as described in Method AD. The crude was purified by column chromatography over silica gel using 25% ethyl acetate-hexane to obtain compound 104.5 as a colorless gum (200 mg, 65% yield). ¹H NMR (400 MHz, DMSO-d₆): δ 8.62 (d, J=4.8 Hz, 1H), 8.04 (d, J=3.6 Hz, 1H), 7.81 (d, J=4.8 Hz, 1H), 7.50-7.40 (m, 2H), 7.22 (t, J=8.5 Hz, 2H), 7.07 (d, J=3.6 Hz, 1H), 6.15-6.0 (m, 1H), 3.50-3.25 (m, 2H), 1.62 (s, 9H), 0.84 (t, J=6.4 Hz, 3H).

Synthesis of (R)—N-ethyl-N-(2,2,2-trifluoro-1-(4-fluorophenyl)ethyl)-1H-pyrrolo[2,3-b]pyridine-4-sulfonamide: N-Boc deprotection of intermediate 104.5 was performed following a method analogous to that described in Method AG. The volatiles were evaporated under reduced pressure and the crude was triturated with 50% diethyl ether-pentane. It was further lyophilized to obtain the compound Example 175 as a yellow solid (80 mg, 98.58% purity, 49% yield). LCMS: m/z found 402.17 [M+H]⁺, rt=3.08 min (Method 2) [Waters Acquity BEH C8 column (1.7 μm, 50×2.1 mm)]; ¹H NMR (400 MHz, DMSO-d₆): δ 12.30 (s, 1H), 8.41 (s, 1H), 7.77 (s, 1H), 7.57 (d, J=3.7 Hz, 1H), 7.44-7.35 (m, 2H), 7.18 (t, J=8.3 Hz, 2H), 6.83 (s, 1H), 6.04 (q, J=8.6 Hz, 1H), 3.46-3.32 (m, 1H), 3.30-3.18 (m, 1H), 0.86 (t, J=6.6 Hz, 3H).

### Example 176

### (R)—N-ethyl-N-(2,2,2-trifluoro-1-(4-fluorophenyl)ethyl)imidazo[1,2-b]pyridazine-3-sulfonamide

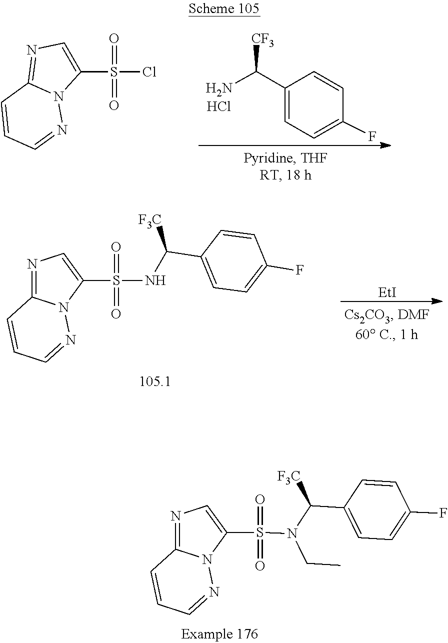 [SMILES: "EtI" ; O=S(=O)(Cl)c1cnc2cccnn12 ; Cl ; N[C@H](c1ccc(F)cc1)C(F)(F)F ; O=S(=O)(N[C@H](c1ccc(F)cc1)C(F)(F)F)c1cnc2cccnn12 ; CCN([C@H](c1ccc(F)cc1)C(F)(F)F)S(=O)(=O)c1cnc2cccnn12]

Synthesis of (R)—N-(2,2,2-trifluoro-1-(4-fluorophenyl)ethyl)imidazo[1,2-b]pyridazine-3-sulfonamide 105.1: Intermediate 105.1 was synthesized from imidazo[1,2-b]pyridazine-3-sulfonyl chloride following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate desired compound 105.1 as a yellow sticky gum (25 mg, 31% yield). ¹H NMR (400 MHz, DMSO-d₆): δ 9.91 (d, J=10.4 Hz, 1H), 8.65-8.55 (m, 1H), 8.20-8.10 (m, 2H), 7.45-7.30 (m, 3H), 6.88 (t, J=7.5 Hz, 2H), 5.35-5.25 (m, 1H).

Synthesis of (R)—N-ethyl-N-(2,2,2-trifluoro-1-(4-fluorophenyl)ethyl)imidazo[1,2-b]pyridazine-3-sulfonamide: The ethylation of intermediate 105.1 was performed following the protocol as described in Method AD. The crude was purified by column chromatography over silica gel using 35% ethyl acetate-hexane to obtain compound Example 176 as an off-white solid (10 mg, 97.25% purity, 33% yield). LCMS: m/z found 383.2 [M+H]⁺, rt=3.18 min (Method 2) [Waters Acquity BEH C8 column (1.7 μm, 50×2.1 mm)]; ¹H NMR (400 MHz, Chloroform-d): δ 8.89 (s, 1H), 8.70 (d, J=1.9 Hz, 1H), 7.86-7.78 (m, 1H), 7.46-7.34 (m, 4H), 5.83 (q, J=8.2 Hz, 1H), 2.74 (s, 3H).

### Example 177

### (R)—N-ethyl-N-(2,2,2-trifluoro-1-(4-fluorophenyl)ethyl)imidazo[1,2-a]pyrimidine-3-sulfonamide

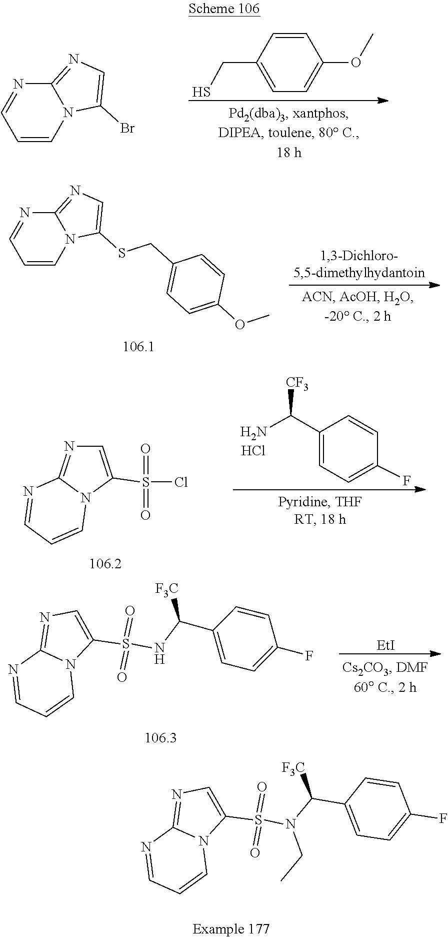

Synthesis of 3-((4-methoxybenzyl)thio)imidazo[1,2-a]pyrimidine 106.1: Intermediate 106.1 was synthesized from 3-bromoimidazo[1,2-a]pyrimidine following a method analogous to that described in Method AA using (4-methoxyphenyl)-methanethiol. The crude residue was purified by column chromatography over silica gel using 30% ethyl acetate in hexane to yield compound 106.1 as a yellow gum (1.3 g, 90% yield). ¹H NMR (400 MHz, DMSO-d₆): δ 8.64-8.56 (m, 2H), 7.74 (s, 1H), 7.10-7.05 (m, 1H), 6.94 (d, J=8.5 Hz, 2H), 6.75-6.70 (m, 2H), 3.92 (s, 2H), 3.67 (s, 3H).

Synthesis of imidazo[1,2-a]pyrimidine-3-sulfonyl chloride 106.2: Sulfonyl chloride 106.2 was synthesized from intermediate 106.1 following the procedure described in Method AB. The crude sulfonyl chloride was used immediately in the forwarding step without further purification.

Synthesis of (R)—N-(2,2,2-trifluoro-1-(4-fluorophenyl)ethyl)imidazo[1,2-a]pyrimidine-3-sulfonamide 106.3: Intermediate 106.3 was synthesized from sulfonyl chloride 106.2 following the protocol as described in Method AC using (R)-2,2,2-trifluoro-1-(4-fluorophenyl)ethan-1-amine hydrochloride. The compound was purified by column chromatography over silica gel using 35% ethyl acetate in hexane to isolate desired compound 106.2 as a yellow sticky gum (120 mg, 27% yield). ¹H NMR (400 MHz, DMSO-d₆): δ 9.90 (d, J=8.8 Hz, 1H), 8.87 (d, J=6.6 Hz, 1H), 8.71 (s, 1H), 8.12 (s, 1H), 7.35-7.26 (m, 3H), 6.86 (t, J=8.6 Hz, 2H), 5.45-5.30 (m, 1H).

Synthesis of (R)—N-ethyl-N-(2,2,2-trifluoro-1-(4-fluorophenyl)ethyl)imidazo[1,2-a]pyrimidine-3-sulfonamide: The ethylation of intermediate 106.3 was preformed following the protocol as described in Method AD. The crude was purified by column chromatography over silica gel using 40% ethyl acetate-hexane to obtain compound Example 177 as an off white solid (12 mg, 99.78% purity). LCMS: m/z found 403.2[M+H]⁺, rt=2.95 min (Method 2) [Waters Acquity BEH C8 column (1.7 μm, 50×2.1 mm)]; ¹H NMR (400 MHz, DMSO-d₆): δ 9.14 (dd, J=1.9, 6.9 Hz, 1H), 8.85-8.79 (m, 1H), 8.50 (s, 1H), 7.46 (dd, J=5.3, 8.6 Hz, 2H), 7.36 (dd, J=4.2, 6.9 Hz, 1H), 7.20 (t, J=8.8 Hz, 2H), 6.08 (q, J=8.7 Hz, 1H), 3.52-3.39 (m, 2H), 0.84 (t, J=6.9 Hz, 3H).

### Example 178

### (R)—N-(1-(4-chlorophenyl)-2,2,2-trifluoroethyl)-N-methylimidazo[1,2-a]pyridine-6-sulfonamide

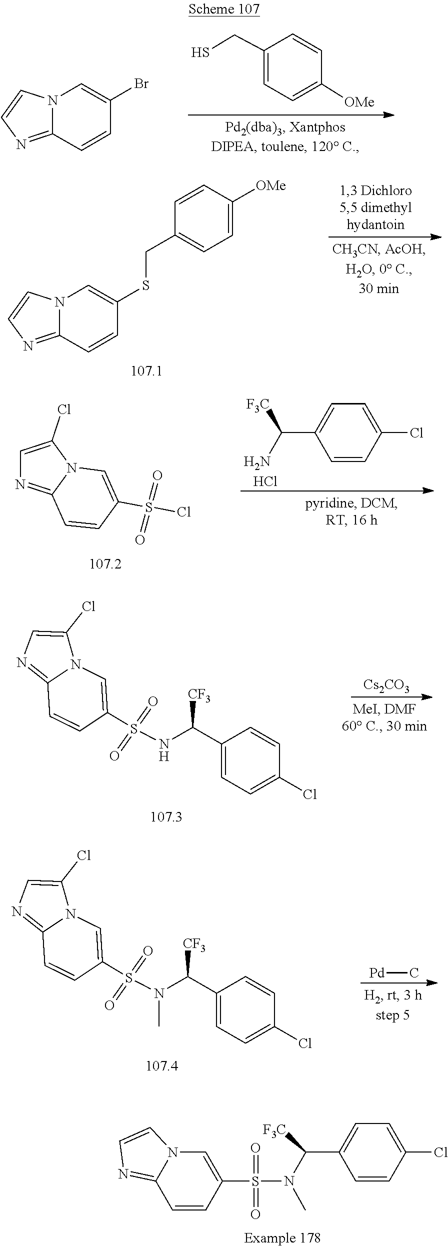

Synthesis of 6-((4-methoxybenzyl)thio)imidazo[1,2-a]pyridine 107.1: Intermediate 107.1 was synthesized from 6-bromoimidazo[1,2-a]pyridine following a method analogous to that described in Method AA using (4-methoxyphenyl)methanethiol. The crude residue was purified by column chromatography over silica gel using 20% ethyl acetate-hexane to get the compound 107.1 as yellow gum. (1.0 g, 78% yield). LCMS: m/z found 271.0 [M+H]⁺, rt=1.53 min (Method 3) [YMC Triart C18 column (3 μm, 33×2.1 mm)].

Synthesis of 3-chloroimidazo[1,2-a]pyridine-6-sulfonyl chloride 107.2: Sulfonyl chloride 107.2 was synthesized from intermediate 107.1 following the procedure described in Method AB. The crude sulfonyl chloride was used immediately in the forwarding step without further purification.

Synthesis of (R)-3-chloro-N-(1-(4-chlorophenyl)-2,2,2-trifluoroethyl)imidazo[1,2-a]pyridine-6-sulfonamide 107.3: Intermediate 107.3 was synthesized from sulfonyl chloride 107.2 following the protocol as described in Method AC using (R)-1-(4-chlorophenyl)-2,2,2-trifluoroethan-1-amine hydrochloride. The compound was purified by column chromatography over silica gel using 35% ethyl acetate in hexane to isolate the desired compound as a yellow gum (130 mg, 15% yield). ¹H NMR (400 MHz, DMSO-d₆): δ 9.63 (d, J=10.6 Hz, 1H), 8.41 (s, 1H), 7.85 (s, 1H), 7.63 (d, J=9.2 Hz, 1H), 7.43-7.29 (m, 3H), 7.23-7.12 (m, 2H), 5.56-5.50 (m, 1H).

Synthesis of (R)-3-chloro-N-(1-(4-chlorophenyl)-2,2,2-trifluoroethyl)-N-methylimidazo[1,2-a]pyridine-6-sulfonamide 107.4: The methylation of intermediate 107.3 was preformed following the protocol as described in Method AD. The crude was purified by column chromatography over silica gel using 20% ethyl acetate in hexane to yield the compound as a colorless gum (100 mg, 75% yield). ¹H NMR (400 MHz, DMSO-d₆): δ 8.76 (s, 1H), 7.72 (s, 1H), 7.80 (d, J=8.8 Hz, 1H), 7.60 (d, J=8.7 Hz, 1H), 7.43 (s, 4H), 6.20-6.15 (m, 1H), 2.85 (m, 4H).

Synthesis of (R)—N-(1-(4-chlorophenyl)-2,2,2-trifluoroethyl)-N-methylimidazo[1,2-a]pyridine-6-sulfonamide: A solution of intermediate 107.4 (100 mg, 0.25 mmol) in ethanol (10 mL) was degassed with argon and to it was added Pd on C (10% w/w) (20 mg). The RM was then stirred under H₂ atmosphere (balloon pressure) at room temperature for 3 h. It was then filtered through sintered funnel and the filtrate was concentrated. The crude was purified by Reverse Phase Prep-HPLC and the compound Example 178 was isolated as white solid (33 mg, 99.50% purity, 35% yield) after lyophilization. RP method: Preparative HPLC was done in Waters BGM 2545 equipped with PDA detector set to multiple-wavelength UV (200-400 nm) detection. Column name: YMC Actus Triart C18 (250×20 mm, 5μ) operating at ambient temperature and flow rate of 16 mL/min. Mobile phase: A=20 mM Ammonium Bicarbonate in water, Mobile phase B=Acetonitrile. Gradient profile: Mobile phase initial composition of 60% A and 40% B, then to 10% A and 90% B in 18 min, then to 5% A and 95% B in 19 min, held in this composition up to 22 min for column washing, then returned to initial composition in 22.5 min and held till 25 min. LCMS: m/z found 404.14 [M+H]⁺, rt=2.84 min (Method 2) [Waters Acquity BEH C8 column (1.7 μm, 50×2.1 mm)]; ¹H NMR (400 MHz, Chloroform-d): δ 8.82 (s, 1H), 7.84-7.73 (m, 3H), 7.48 (d, J=9.1 Hz, 1H), 7.40 (s, 4H), 5.87 (q, J=8.2 Hz, 1H), 2.73 (s, 3H).

### Example 179

### (R)—N-(1-(4-chlorophenyl)-2,2,2-trifluoroethyl)-N-methyl-[1,2,5]thiadiazolo[3,4-b]pyridine-6-sulfonamide

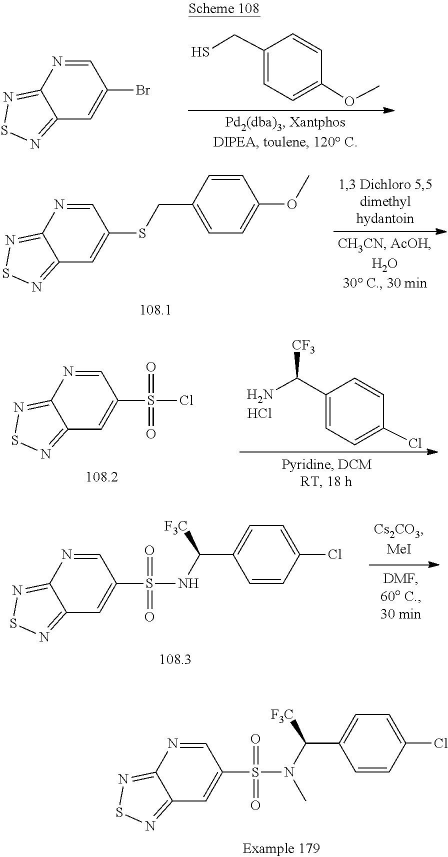

Synthesis of 6-((4-methoxybenzyl)thio)-[1,2,5]thiadiazolo[3,4-b]pyridine 108.1: Intermediate 108.1 was synthesized from 6-bromo-[1,2,5]thiadiazolo[3,4-b]pyridine following a method analogous to that described in Method AA using (4-methoxyphenyl)methanethiol. The crude was purified by column chromatography over silica gel using 30% ethyl acetate in hexane to yield compound 108.1 as a yellow gum (550 mg, 80% yield). ¹H NMR (400 MHz, DMSO-d₆): δ 8.98 (d, J=2.3 Hz, 1H), 8.36 (d, J=2.3 Hz, 1H), 7.45-7.35 (m, 2H), 6.89 (d, J=8.6 Hz, 2H), 4.45 (s, 2H), 3.71 (s, 3H).

Synthesis of [1,2,5]thiadiazolo[3,4-b]pyridine-6-sulfonyl chloride 108.2: Sulfonyl chloride 108.2 was synthesized from intermediate 108.1 following the procedure described in Method AB. The crude sulfonyl chloride was used immediately in the forwarding step without further purification.

Synthesis of (R)—N-(1-(4-chlorophenyl)-2,2,2-trifluoroethyl)-[1,2,5]thiadiazolo[3,4-b]pyridine-6-sulfonamide 108.3: Intermediate 108.3 was synthesized from sulfonyl chloride 108.2 following the protocol as described in Method AC using (R)-1-(4-chlorophenyl)-2,2,2-trifluoroethan-1-amine hydrochloride. The compound was purified by column chromatography over silica gel using 35% ethyl acetate in hexane to isolate the desired compound as a yellow gum (100 mg, 23% yield). ¹H NMR (400 MHz, DMSO-d₆): δ 9.91 (d, J=7.2 Hz, 1H), 9.20 (d, J=2.0 Hz, 1H), 8.73 (d, J=1.6 Hz, 1H), 7.38 (d, J=8.0 Hz, 2H), 7.16 (d, J=8.0 Hz, 2H), 5.70-5.50 (m, 1H).

Synthesis of (R)—N-(1-(4-chlorophenyl)-2,2,2-trifluoroethyl)-N-methyl-[1,2,5]thiadiazolo[3,4-b]pyridine-6-sulfonamide: The methylation of intermediate 108.3 was preformed following the protocol as described in Method AD. The crude was purified by column chromatography over silica gel using 30% ethyl acetate-hexane to yield Example 179 as a light brown sticky gum (55 mg, 99.93% purity, 26% yield). LCMS: m/z found 423.10 [M+H]⁺, rt=3.18 min (Method 2) [Waters Acquity BEH C8 column (1.7 μm, 50×2.1 mm)]; ¹H NMR (400 MHz, Chloroform-d): δ 9.41 (d, J=2.0 Hz, 1H), 8.87 (d, J=2.4 Hz, 1H), 7.42 (s, 4H), 5.93 (q, J=8.4 Hz, 1H), 2.81 (s, 3H).

### Example 180

### (R)—N-ethyl-2-methyl-N-(2,2,2-trifluoro-1-(4-fluorophenyl)ethyl)imidazo[1,2-b]pyridazine-6-sulfonamide

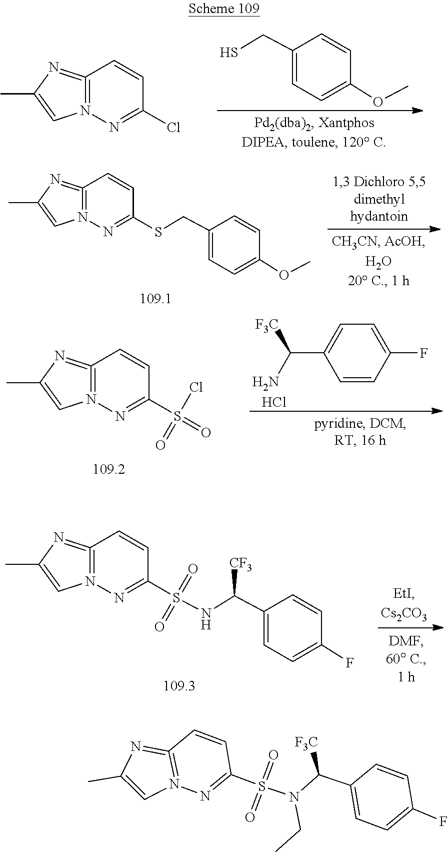

Synthesis of 6-((4-methoxybenzyl)thio)-2-methylimidazo[1,2-b]pyridazine 109.1: Intermediate 109.1 was synthesized 6-chloro-2-methylimidazo[1,2-b]pyridazine following a method analogous to that described in Method AA using (4-methoxyphenyl)methanethiol. The volatiles were evaporated under reduced pressure and triturated with hexane to provide compound 109.1 (1.5 g, 44% yield) as yellow gum. ¹H NMR (400 MHz, DMSO-d6): δ 7.99 (s, 1H), 7.81 (d, J=9.4 Hz, 1H), 7.38 (d, J=8.4 Hz, 2H), 7.02 (t, J=9.4 Hz, 1H), 6.86 (d, J=8.56 Hz, 2H), 4.37 (s, 2H), 3.71 (s, 3H), 2.34 (s, 3H).

Synthesis of 2-methylimidazo[1,2-b]pyridazine-6-sulfonyl chloride 109.2: Sulfonyl chloride 109.2 was synthesized from intermediate 109.1 following the procedure described in Method AB. The crude sulfonyl chloride was used immediately in the forwarding step without further purification.

Synthesis of (R)-2-methyl-N-(2,2,2-trifluoro-1-(4-fluorophenyl)ethyl)imidazo[1,2-b]pyridazine-6-sulfonamide 109.3: Intermediate 109.3 was synthesized from sulfonyl chloride 109.2 following the protocol as described in Method AC using (R)-2,2,2-trifluoro-1-(4-fluorophenyl)ethan-1-amine hydrochloride. The crude was purified by column chromatography over silica gel using 35% ethyl acetate in hexane to isolate the sulfonamide 109.3 as a yellow sticky gum (100 mg, 15% yield). LCMS: m/z found 389.1 (M+H)+, rt=1.74 min (Method 3) [YMC Triart C18 column (3 μm, 33×2.1 mm)].

Synthesis of (R)—N-ethyl-2-methyl-N-(2,2,2-trifluoro-1-(4-fluorophenyl)ethyl)imidazo[1,2-b]pyridazine-6-sulfonamide: The ethylation of intermediate 109.3 was preformed following the protocol as described in Method AD. The crude was purified by column chromatography over silica gel using 1:4 ethyl acetate-hexane to obtain compound Example 180 as a colorless oil (15 mg, 98.48% purity, 25% yield). LCMS: m/z found 417.1 [M+H]⁺, rt=2.82 min (Method 9) [Waters Xbridge C18 column (3.5 μm, 50×3 mm)]; ¹H NMR (400 MHz, DMSO-d6): δ 8.28-8.21 (m, 2H), 7.66 (d, J=9.6 Hz, 1H), 7.61-7.53 (m, 2H), 7.24 (t, J=8.4 Hz, 2H), 6.01 (q, J=8.8 Hz, 1H), 3.48-3.37 (m, 2H), 2.47 (s, 3H), 0.94 (t, J=6.8 Hz, 3H).

### Example 181

### (R)—N-ethyl-N-(2,2,2-trifluoro-1-(4-fluorophenyl)ethyl)-1H-pyrrolo[2,3-c]pyridine-5-sulfonamide

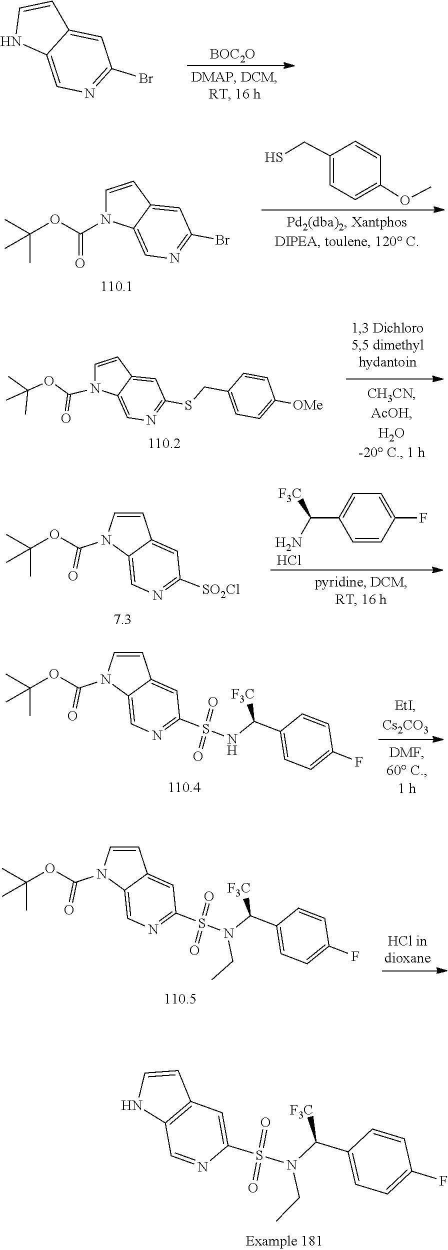

Synthesis of tert-butyl 5-bromo-1H-pyrrolo[2,3-c]pyridine-1-carboxylate 110.1: Intermediate 110.1 was synthesized from 4-iodo-1H-pyrrolo[2,3-b]pyridine following a method analogous to that described in Method AF. The crude was purified by column chromatography over silica gel using 20% ethyl acetate-hexane to obtain the compound 110.1 was isolated as a white solid (1.40 g, 93% yield). ¹H NMR (400 MHz, DMSO-d₆): δ 9.00 (s, 1H), 7.95 (d, J=3.6 Hz, 1H), 7.91 (s, 1H), 6.76 (d, J=3.6 Hz, 1H), 1.64 (s, 9H).

Synthesis of tert-butyl 5-((4-methoxybenzyl)thio)-1H-pyrrolo[2,3-c]pyridine-1-carboxylate 110.2: Intermediate 110.2 was synthesized from intermediate 110.1 following a method analogous to that described in Method AA using (4-methoxyphenyl)methanethiol. The volatiles were evaporated under reduced pressure and triturated with hexane to get intermediate 110.2 as a yellow sticky solid (1.40 g, 80% yield). ¹H NMR (400 MHz, DMSO-d₆): δ 9.11 (s, 1H), 7.81 (m, 1H), 7.54 (s, 1H), 7.30 (d, J=8.6 Hz, 2H), 6.83 (d, J=8.6 Hz, 2H), 6.66 (d, J=3.2 Hz, 1H), 4.36 (s, 2H), 3.70 (s, 3H), 1.64 (s, 9H).

Synthesis of tert-butyl 5-(chlorosulfonyl)-1H-pyrrolo[2,3-c]pyridine-1-carboxylate 110.3: Sulfonyl chloride 110.3 was synthesized from intermediate 110.2 following the procedure described in Method AB. The crude sulfonyl chloride was used immediately in the forwarding step without further purification.

Synthesis of tert-butyl (R)-5-(N-(2,2,2-trifluoro-1-(4-fluorophenyl)ethyl)sulfamoyl)-1H-pyrrolo[2,3-c]pyridine-1-carboxylate 110.4: Intermediate 110.4 was synthesized from sulfonyl chloride 110.3 following the protocol as described in Method AC using (R)-2,2,2-trifluoro-1-(4-fluorophenyl)ethan-1-amine hydrochloride. The crude was purified by column chromatography over silica gel using 35% ethyl acetate in hexane to isolate the sulfonamide 110.4 as yellow sticky gum (300 mg, 57% yield). LCMS: m/z found 474.0 (M+H)+, rt=3.69 min (Method 4) [Waters Xbridge C18 column (5 μm, 50×4.6 mm)].

Synthesis of tert-butyl (R)-5-(N-ethyl-N-(2,2,2-trifluoro-1-(4-fluorophenyl)ethyl)sulfamoyl)-1H-pyrrolo[2,3-c]pyridine-1-carboxylate 110.5: The ethylation of intermediate 110.4 was preformed following the protocol as described in Method AD. The crude was purified by column chromatography over silica gel using 1:4 ethyl acetate-hexane to obtain intermediate 110.5 as a colorless oil (125 mg, 39% yield). LCMS: m/z found 502.0 [M+H]⁺, rt=4.08 min (Method 4) [Waters Xbridge C18 column (5 μm, 50×4.6 mm)].

Synthesis of (R)—N-ethyl-N-(2,2,2-trifluoro-1-(4-fluorophenyl)ethyl)-1H-pyrrolo[2,3-c]pyridine-5-sulfonamide: N-Boc deprotection of intermediate 110.4 was performed following a method analogous to that described in Method AG. The volatiles were evaporated under reduced pressure and the crude was purified by column chromatography over silica gel using 35% ethyl acetate in hexane to isolate the compound Example 181 as a white solid (55 mg, 97.93% purity, 55% yield). LCMS: m/z found 402.3 [M+H]⁺, rt=2.22 min (Method 25) [Waters Acquity BEH C18 column (1.7 μm, 50×2.1 mm)]; ¹H NMR (400 MHz, DMSO-d₆): δ 12.16 (s, 1H), 8.82 (s, 1H), 8.28 (s, 1H), 7.82 (d, J=3.0 Hz, 1H), 7.52 (dd, J=5.4, 8.6 Hz, 2H), 7.17 (t, J=8.8 Hz, 2H), 6.74 (d, J=2.9 Hz, 1H), 5.91 (q, J=8.8 Hz, 1H), 3.28-3.14 (m, 2H), 0.87 (t, J=7.0 Hz, 3H).

### Example 182

### (R)—N-ethyl-N-(2,2,2-trifluoro-1-(4-fluorophenyl)ethyl)-1H-indazole-4-sulfonamide

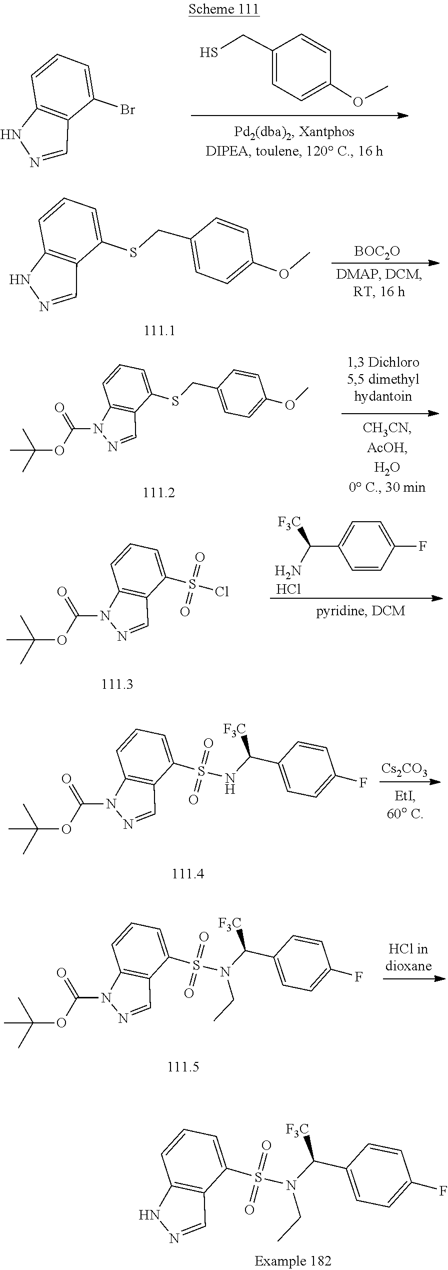

Synthesis of 4-((4-methoxybenzyl)thio)-1H-indazole 111.1: Intermediate 111.1 was synthesized from 4-bromo-1H-indazole following a method analogous to that described in Method AA using (4-methoxyphenyl)methanethiol. The volatiles were evaporated under reduced pressure and the crude was purified by combi flash column chromatography using 40% ethyl acetate in hexane as eluent to afford 111.1 (1.2 g, 87% yield). LCMS: m/z found 271.0 [M+H]⁺, rt=2.01 min (Method 25) [Waters Acquity BEH C18 column (1.7 μm, 50×2.1 mm)].

Synthesis of tert-butyl 4-((4-methoxybenzyl)thio)-1H-indazole-1-carboxylate 111.2: Intermediate 111.2 was synthesized from intermediate 111.2 following a method analogous to that described in Method AF. The crude was purified by column chromatography over silica gel using 20% ethyl acetate-hexane to obtain the compound 111.2. The crude was purified by column chromatography using 10% EA in Hexane as eluent to afford desire product 111.2 (1.07 g, 65% yield). LCMS: m/z found 371.2 [M+H]⁺, rt=2.17 min (Method 8) [Waters Xbridge C18 column (3.5 μm, 50×3 mm)].

Synthesis of tert-butyl 4-(chlorosulfonyl)-1H-indazole-1-carboxylate 111.3: Sulfonyl chloride 111.3 was synthesized from intermediate 111.2 following the procedure described in Method AB. The crude sulfonyl chloride was used immediately in the forwarding step without further purification.

Synthesis of tert-butyl (R)-4-(N-(2,2,2-trifluoro-1-(4-fluorophenyl)ethyl)sulfamoyl)-1H-indazole-1-carboxylate 111.4: Intermediate 111.4 was synthesized from sulfonyl chloride 111.3 following the protocol as described in Method AC using (R)-1-(4-chlorophenyl)-2,2,2-trifluoroethan-1-amine hydrochloride. Crude compound was purified by column chromatography using 20% ethyl acetate in hexane as eluent to afford desire product 8.4 as off white solid (150 mg, 25% yield). LCMS: m/z found 474.1 [M+H]⁺, rt=1.99 min (Method 8) [Waters Xbridge C18 column (3.5 μm, 50×3 mm)].

Synthesis of tert-butyl (R)-4-(N-ethyl-N-(2,2,2-trifluoro-1-(4-fluorophenyl)ethyl)sulfamoyl)-1H-indazole-1-carboxylate 111.5: The ethylation of intermediate 111.5 was performed following the protocol as described in Method AD. The crude was purified by column chromatography over silica gel using 20% ethyl acetate-hexane to obtain compound 111.5 as sticky gum (90 mg, 56% yield). LCMS: m/z found 502.1 [M+H]⁺, rt=2.25 min (Method 9) [Waters Xbridge C18 column (3.5 μm, 50×3 mm)].

Synthesis of (R)—N-ethyl-N-(2,2,2-trifluoro-1-(4-fluorophenyl)ethyl)-1H-indazole-4-sulfonamide: N-Boc deprotection of intermediate 111.5 was performed following a method analogous to that described in Method AG. The volatiles were evaporated under reduced pressure and the crude was triturated with 50% diethyl ether-pentane. It was further purified by Reverse Phase Prep-HPLC to obtain the compound Example 182 as a sticky gum (37.46 mg, 99.94% purity, 52% yield). RP Method: Preparative HPLC Was done in Waters BGM 2545 equipped with Waters PDA detector 2998 set to multiple-wavelength UV (200-400 nm) detection. Column name: YMC Actus Triart C18 (250×20 mm, 5μ) operating at ambient temperature and flow rate of 16 mL/min. Mobile phase: A=20 mM Ammonium Bicarbonate in water, B=Acetonitrile. Gradient profile: Mobile phase initial composition of 60% A and 40% B, then to 10% A and 90% B in 18 min. then to 5% A and 95% B in 19 min held in this composition up to 22 min for column washing, then returned to initial composition in 22.5 min and held till 25 min. LCMS: m/z found 400.14 [M−H]⁺, rt=3.02 min (Method 2) [Waters Acquity BEH C8 column (1.7 μm, 50×2.1 mm)]. ¹H NMR (400 MHz, DMSO-d₆): δ 13.65 (s, 1H), 8.38 (s, 1H), 7.90 (d, J=8.3 Hz, 1H), 7.76 (d, J=7.2 Hz, 1H), 7.52 (t, J=7.8 Hz, 1H), 7.37 (dd, J=5.3, 8.6 Hz, 2H), 7.17 (t, J=8.8 Hz, 2H), 6.08 (q, J=8.7 Hz, 1H), 3.45-3.32 (m, 1H), 3.31-3.17 (m, 1H), 0.82 (t, J=7.0 Hz, 3H).

### Example 183

### (R)—N-methyl-N-(2,2,2-trifluoro-1-(4-fluorophenyl)ethyl)pyrazolo[1,5-a]pyrimidine-6-sulfonamide

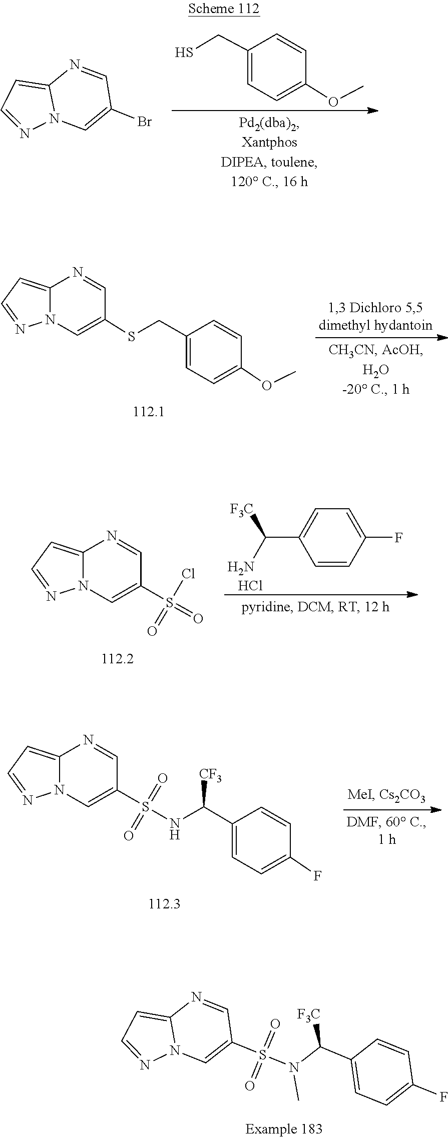

Synthesis of 6-((4-methoxybenzyl)thio)pyrazolo[1,5-a]pyrimidine 112.1 Intermediate 112.1 was synthesized from 6-bromopyrazolo[1,5-a]pyrimidine following Buchwald protocol as described in Method AA using (4-methoxyphenyl)methanethiol. The crude was purified by column chromatography over silica gel using 50% ethyl acetate in hexane to obtain desired compound 112.1 as a yellow sticky gum (600 mg, 84% yield). LCMS: m/z found 272.34 [M+H⁺], rt=2.48 min (Method 9) [Waters Xbridge C18 column (3.5 μm, 50×3 mm)].

Synthesis of pyrazolo[1,5-a]pyrimidine-6-sulfonyl chloride 112.2: Sulfonyl chloride 112.2 was synthesized from intermediate 112.1 following the protocol as described in Method AB. The crude sulfonyl chloride 112.2 was used in the next sulfonamidation step without further purification.

Synthesis of (R)—N-(2,2,2-trifluoro-1-(4-fluorophenyl)ethyl)pyrazolo[1,5-a]pyrimidine-6-sulfonamide 112.3: Intermediate 112.3 was synthesized following the protocol as described in Method AC using (R)-2,2,2-trifluoro-1-(4-fluorophenyl)ethan-1-amine hydrochloride. The crude was purified by column chromatography over silica gel using 30% ethyl acetate in hexane to obtain desired sulfonamide 112.3 as a colourless sticky gum. LCMS: m/z found 375.1 [M+H]⁺, rt=3.28 min (Method 14) [Waters Acquity BEH C18 column (1.7 μm, 50×2.1 mm)].

Synthesis of (R)—N-methyl-N-(2,2,2-trifluoro-1-(4-fluorophenyl)ethyl)pyrazolo[1,5-a]pyrimidine-6-sulfonamide: The methylation of intermediate 112.3 was performed following the protocol as described in Method AD. The crude was purified by column chromatography over silica gel using 30% ethyl acetate in hexane to obtain the desired compound Example 183 (20 mg, 30% yield, 95.5% purity) as a white solid. LCMS: m/z found 389.13 [M+H]⁺, rt=3.11 min (Method 2) [Waters Acquity BEH C8 column (1.7 μm, 50×2.1 mm)]; 1H NMR (400 MHz, CDCl3): δ 9.19 (s, 1H), 8.71 (s, 1H), 8.32 (s, 1H), 7.54-7.41 (m, 2H), 7.13 (t, J=8.4 Hz, 2H), 6.86 (s, 1H), 5.91 (q, J=8.1 Hz, 1H), 2.77 (s, 3H).

### Example 184

### (R)—N-ethyl-2-methyl-N-(2,2,2-trifluoro-1-(4-fluorophenyl)ethyl)imidazo[1,2-b]pyridazine-6-sulfonamide

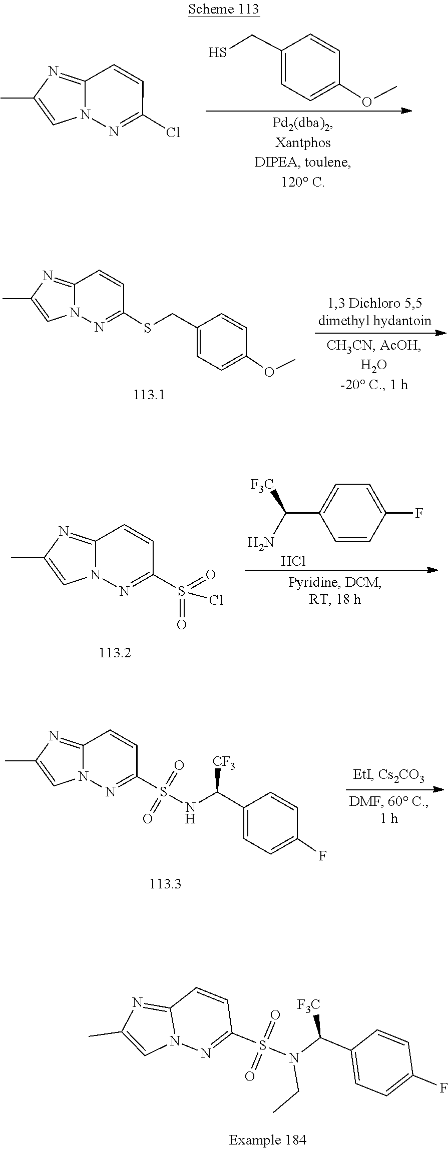

Synthesis of 6-((4-methoxybenzyl)thio)-2-methylimidazo[1,2-b]pyridazine 113.1: Intermediate 113.1 was synthesized 6-chloro-2-methylimidazo[1,2-b]pyridazine following a method analogous to that described in Method AA using (4-methoxyphenyl)methanethiol. The volatiles were evaporated under reduced pressure and triturated with hexane to get intermediate 113.1 (1.50 g, 44% yield). ¹H NMR (400 MHz, DMSO-d6): δ 7.99 (s, 1H), 7.81 (d, J=9.4 Hz, 1H), 7.38 (d, J=8.4 Hz, 2H), 7.02 (t, J=9.4 Hz, 1H), 6.86 (d, J=8.5 Hz, 2H), 4.37 (s, 2H), 3.71 (s, 3H), 2.34 (s, 3H).

Synthesis of 2-methylimidazo[1,2-b]pyridazine-6-sulfonyl chloride 113.2: Sulfonyl chloride 113.2 was synthesized from intermediate 113.1 following the procedure described in Method AB. The crude sulfonyl chloride was used immediately in the forwarding step without further purification.

Synthesis of (R)-2-methyl-N-(2,2,2-trifluoro-1-(4-fluorophenyl)ethyl)imidazo[1,2-b]pyridazine-6-sulfonamide 113.3: Intermediate 113.3 was synthesized from sulfonyl chloride 113.2 following the protocol as described in Method AC using (R)-2,2,2-trifluoro-1-(4-fluorophenyl)ethan-1-amine hydrochloride. The crude was purified by column chromatography over silica gel using 35% ethyl acetate in hexane to isolate the sulfonamide 113.3 as a yellow sticky gum (100 mg, 15% yield). LCMS: m/z found 389.1 (M+H)⁺, rt=1.74 min (Method 3) [YMC Triart C18 column (3 μm, 33×2.1 mm)].

Synthesis of (R)—N-ethyl-2-methyl-N-(2,2,2-trifluoro-1-(4-fluorophenyl)ethyl)imidazo[1,2-b]pyridazine-6-sulfonamide: The ethylation of intermediate 113.3 was preformed following the protocol as described in Method AD. The crude was purified by column chromatography over silica gel using 1:4 ethyl acetate-hexane to obtain compound Example 184 as a colorless oil (15 mg, 98.48% purity, 25% yield). LCMS: m/z found 417.1 [M+H]⁺, rt=2.82 min (Method 9) [Waters Xbridge C18 column (3.5 μm, 50×3 mm)]. ¹H NMR (400 MHz, DMSO-d6): δ 8.28-8.21 (m, 2H), 7.66 (d, J=9.6 Hz, 1H), 7.61-7.53 (m, 2H), 7.24 (t, J=8.4 Hz, 2H), 6.01 (q, J=8.8 Hz, 1H), 3.48-3.37 (m, 2H), 2.47 (s, 3H), 0.94 (t, J=6.8 Hz, 3H).

### Example 185

### (R)—N-ethyl-N-(2,2,2-trifluoro-1-(4-fluorophenyl)ethyl)-1H-benzo[d]imidazole-6-sulfonamide

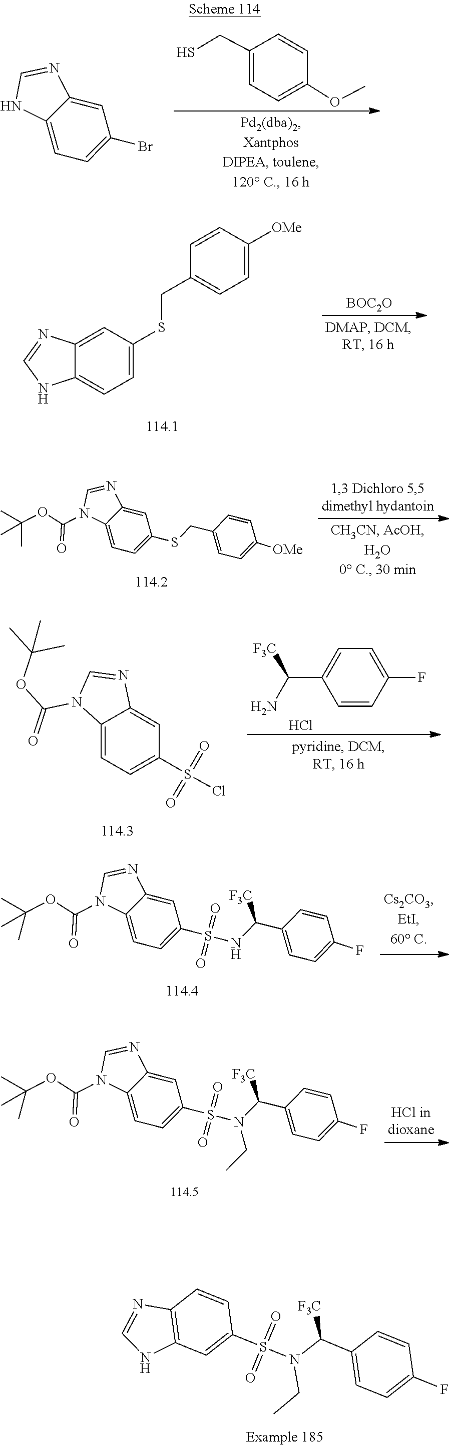

Synthesis of 5-((4-methoxybenzyl)thio)-1H-benzo[d]imidazole: Intermediate 114.1 was synthesized from 5-bromo-1H-benzo[d]imidazole following Buchwald protocol as described in Method AA using (4-methoxyphenyl)methanethiol. The crude was purified by column chromatography over silica gel using 50% ethyl acetate in hexane to obtain desired compound 114.1 as yellow sticky gum (1.5 g, 60% yield). LCMS: m/z found 271.01 [M+H⁺], rt=1.54 min (Method 3) [YMC Triart C18 column (3 μm, 33×2.1 mm)).

Synthesis of tert-butyl 5-((4-methoxybenzyl)thio)-1H-benzo[d]imidazole-1-carboxylate 114.2: Intermediate 114.2 was synthesized from intermediate 114.1 following a method analogous to that described in Method AF. The crude was purified by column chromatography over silica gel using 20% ethyl acetate-hexane to obtain compound 114.2. (1.10 g, 60% yield). LCMS: m/z found 371.1 [M+H]⁺, rt=2.82 min (Method 2) Waters Acquity BEH C8 column (1.7 μm, 50×2.1 mm).

Synthesis of tert-butyl 5-(chlorosulfonyl)-1H-benzo[d]imidazole-1-carboxylate 114.3: Intermediate 114.3 was synthesized following the protocol as described in Method AB. The crude sulfonyl chloride 114.3 was used in the next sulfonamidation step without further purification.

Synthesis of tert-butyl (R)-5-(N-(2,2,2-trifluoro-1-(4-fluorophenyl)ethyl)sulfamoyl)-1H-benzo[d]imidazole-1-carboxylate 114.4: Intermediate 114.4 was synthesized from sulfonyl chloride 114.3 following the protocol as described in Method AC using (R)-2,2,2-trifluoro-1-(4-fluorophenyl)ethan-1-amine hydrochloride. Crude compound was purified by column chromatography using 20% ethyl acetate in hexane as eluent to afford desire product 114.4 as off white solid (250 mg, 11% yield). LCMS: m/z found (474.1 [M+H]⁺); rt=1.96 min (Method 8) Waters Xbridge C18 column (3.5 μm, 50×3 mm)).

Synthesis of tert-butyl (R)-5-(N-ethyl-N-(2,2,2-trifluoro-1-(4-fluorophenyl)ethyl)sulfamoyl)-1H-benzo[d]imidazole-1-carboxylate 114.5: The ethylation of intermediate 114.4 was preformed following the protocol as described in Method AD. The crude was purified by column chromatography over silica gel using 20% ethyl acetate-hexane to obtain compound 114.5 as a sticky gum (150 mg, 67% yield). ¹H NMR (400 MHz, DMSO-d₆): δ 8.99-8.80 (m, 1H), 8.35-8.28 (m, 1H), 8.03-7.88 (m, 1H), 7.52-7.38 (m, 2H), 7.26-7.17 (m, 2H), 6.26-5.93 (m, 1H), 3.73-2.68 (m, 2H), 1.66 (s, 9H), 0.99-0.88 (m, 3H).

Synthesis of (R)—N-ethyl-N-(2,2,2-trifluoro-1-(4-fluorophenyl)ethyl)-1H-benzo[d]imidazole-6-sulfonamide: N-Boc deprotection of intermediate 114.5 was performed following a method analogous to that described in Method AG. The volatiles were evaporated under reduced pressure and the crude was triturated with 50% diethyl ether-pentane. It was further lyophilized to obtain the compound Example 185 as a white solid (70 mg, 96.70% purity, 67% yield). LCMS: m/z found 402.16 [M+H]⁺, rt=2.85 min (Method 2) [Waters Acquity BEH C8 column (1.7 μm, 50×2.1 mm)]; ¹H NMR (400 MHz, Chloroform-d): δ 8.32 (d, J=14.8 Hz, 2H), 7.83 (d, J=8.4 Hz, 1H), 7.76 (d, J=8.8 Hz, 1H), 7.44 (dd, J=5.1, 8.5 Hz, 2H), 7.05 (t, J=8.4 Hz, 2H), 5.88 (q, J=8.4 Hz, 1H), 3.43-3.25 (m, 1H), 3.19-3.05 (m, 2H), 0.90 (t, J=7.2 Hz, 3H).

### Example 186 and Example 187

### (R)—N-ethyl-N-(2,2,2-trifluoro-1-(4-fluorophenyl)ethyl)imidazo[1,2-a]pyridine-3-sulfonamide (Example 186) and (R)—N-(1-(4-chlorophenyl)-2,2,2-trifluoroethyl)-N-methylimidazo[1,2-a]pyridine-3-sulfonamide (Example 187)

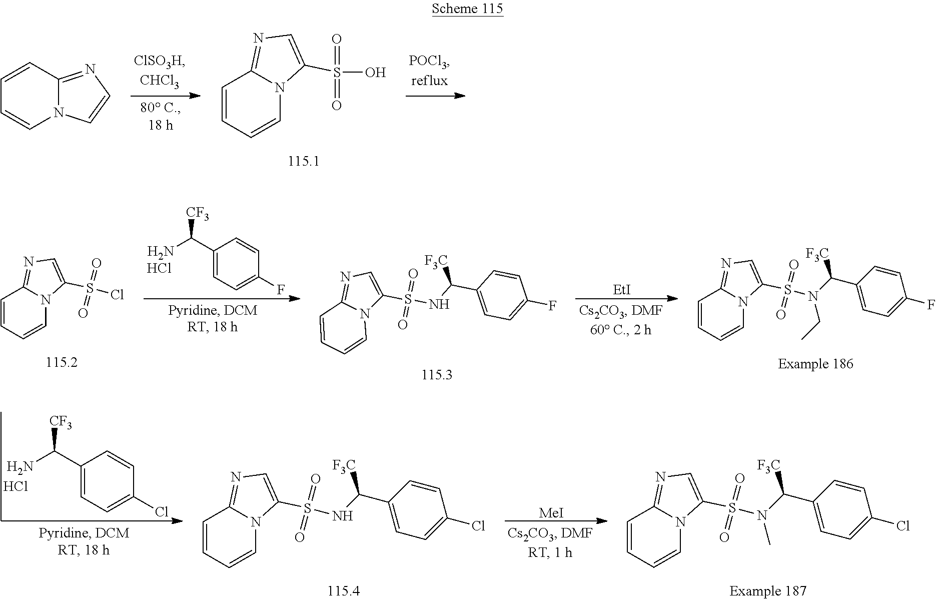

Synthesis of imidazo[1,2-a]pyridine-3-sulfonic acid 115.1: To a stirred suspension of compound imidazo[1,2-a]pyridine (1.0 g, 8.46 mmol) in CHCl₃ (25 mL), a solution of chlorosulfonic acid (1.7 ml, 25.39 mmol) in CHCl₃ (25 mL) was added dropwise at 0° C. and then the resultant mixture was refluxed for 18 h. The reaction mixture was cooled to room temperature and then concentrated under reduced pressure to afford a pale brown sticky liquid which was triturated with ethanol and diethylether to afford an off-white solid which was filtered and dried under vacuum to provide compound 115.1 (850 mg, 50% yield). The solid was used in the forwarding step without purification.

Synthesis of imidazo[1,2-a]pyridine-3-sulfonyl chloride 115.2: Compound 115.1 (1.60 g, 8.07 mmol) was taken in a 100 mL round bottomed flask and POCl₃ (50 ml) was added to it and the mixture was refluxed for 18 h. The reaction mass was cooled, concentrated under reduced pressure, and then partitioned between DCM (250 ml) and water (150 ml). The organic layer was dried over anhydrous MgSO₄, filtered, and concentrated under reduced pressure to afford crude compound 115.2 (1.70 g) which was used in next step without any purification.

Synthesis of (R)—N-(2,2,2-trifluoro-1-(4-fluorophenyl)ethyl)imidazo[1,2-a]pyridine-3-sulfonamide 115.3: Intermediate 115.3 was synthesized from sulfonyl chloride 115.2 following the protocol as described in Method AC using (R)-2,2,2-trifluoro-1-(4-fluorophenyl)ethan-1-amine hydrochloride. Crude compound was purified by column chromatography using 20% ethyl acetate in hexane as eluent to afford desire product 115.3 as off white solid (50 mg, 11% yield). LCMS: m/z found 374.0 (M+H)+, rt=3.42 min (Method 4) [Waters Xbridge C18 column (5 μm, 50×4.6 mm)].

Synthesis of (R)—N-ethyl-N-(2,2,2-trifluoro-1-(4-fluorophenyl)ethyl)imidazo[1,2-a]pyridine-3-sulfonamide: The ethylation of intermediate 115.3 was preformed following the protocol as described in Method AD. The crude was purified by column chromatography over silica gel using 30% ethyl acetate-hexane to obtain Example 186 as an off-white solid (5 mg, 98.5% purity, 15% yield). LCMS: m/z found 383.2 [M+H]⁺, rt=3.18 min (Method 2) [Waters Acquity BEH C8 column (1.7 μm, 50×2.1 mm)]; ¹H NMR (400 MHz, Chloroform-d): δ 8.89 (s, 1H), 8.70 (d, J=1.9 Hz, 1H), 7.86-7.78 (m, 1H), 7.46-7.34 (m, 4H), 5.83 (q, J=8.2 Hz, 1H), 2.74 (s, 3H).

Synthesis of (R)—N-(1-(4-chlorophenyl)-2,2,2-trifluoroethyl)imidazo[1,2-a]pyridine-3-sulfonamide 115.4: Intermediate 115.4 was synthesized from sulfonyl chloride 115.2 following the protocol as described in Method AC using (R)-1-(4-chlorophenyl)-2,2,2-trifluoroethan-1-amine hydrochloride. Crude compound was purified by column chromatography using 20% ethyl acetate in hexane as eluent to afford desire product 115.4 as off white solid (50 mg, 11% yield). LCMS: m/z found 390.0 (M+H)+, rt=3.42 min (Method 4) [Waters Xbridge C18 column (5 μm, 50×4.6 mm)].

Synthesis of (R)—N-(1-(4-chlorophenyl)-2,2,2-trifluoroethyl)-N-methylimidazo[1,2-a]pyridine-3-sulfonamide: The methylation of intermediate 115.4 was performed following the protocol as described in Method AD at RT. The crude was purified by column chromatography over silica gel using 30% ethyl acetate-hexane to obtain compound Example 187 as an off-white solid (13 mg, 99.30% purity, 25% yield). LCMS: m/z found 404.17 [M+H]⁺, rt=3.32 min (Method 2) [Waters Acquity BEH C8 column (1.7 μm, 50×2.1 mm)]; ¹H NMR (400 MHz, DMSO-d₆): δ 8.72 (d, J=6.8 Hz, 1H), 8.30 (s, 1H), 7.83 (d, J=9.0 Hz, 1H), 7.61 (t, J=8.8 Hz, 1H), 7.43 (q, J=8.6 Hz, 4H), 7.24 (t, J=6.6 Hz, 1H), 6.13 (q, J=8.7 Hz, 1H), 2.81 (s, 3H).

### Example 188

### (R)—N-ethyl-N-(2,2,2-trifluoro-1-(4-fluorophenyl)ethyl)-1H-pyrazolo[4,3-b]pyridine-5-sulfonamide

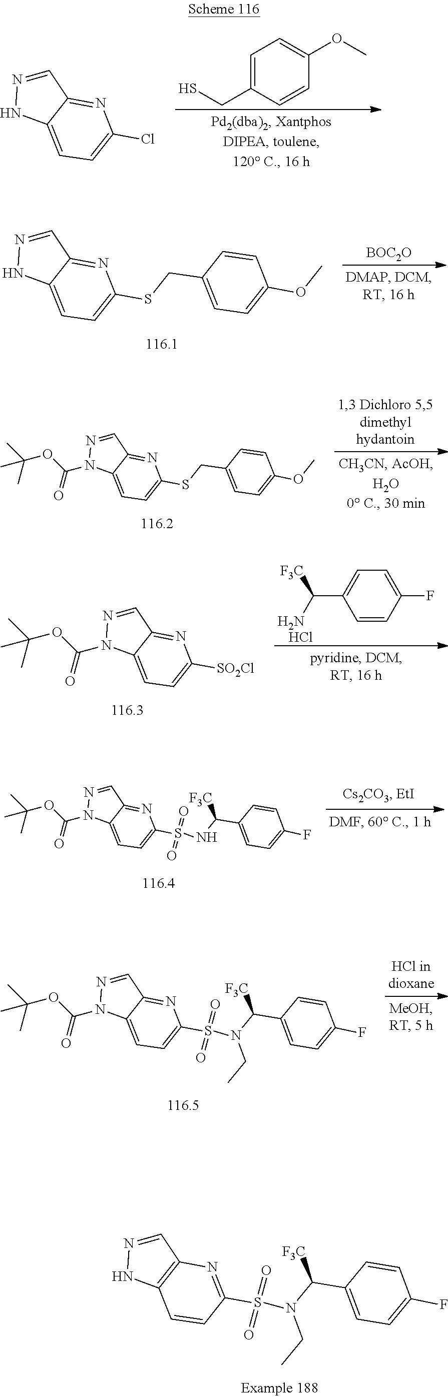

Synthesis of 5-((4-methoxybenzyl)thio)-1H-pyrazolo[4,3-b]pyridine 116.1: Intermediate 116.1 was synthesized from 5-chloro-1H-pyrazolo[4,3-b]pyridine following a method analogous to that described in Method AA using (4-methoxyphenyl)methanethiol. The volatiles were evaporated under reduced pressure and triturated with hexane to get intermediate 116.2 as a yellow sticky solid (1.0 g, 85% yield). LCMS: m/z found 272.0 [M+H]⁺, rt=1.67 min (Method 3) [YMC Triart C18 column (3 μm, 33×2.1 mm)].

Synthesis of tert-butyl 5-((4-methoxybenzyl)thio)-1H-pyrazolo[4,3-b]pyridine-1-carboxylate 116.2: Intermediate 116.2 was synthesized from intermediate 116.1 following a method analogous to that described in Method AF. The crude residue was purified by column chromatography over silica gel using 20% ethyl acetate-hexane to obtain the compound 116.1 was isolated as a white solid (1.0 g, 73% yield). LCMS: m/z found 372.3 [M+H]⁺, rt=2.41 min (Method 25) [Waters Acquity BEH C18 column (1.7 μm, 50×2.1 mm)].

Synthesis of tert-butyl 5-(chlorosulfonyl)-1H-pyrazolo[4,3-b]pyridine-1-carboxylate 116.3: Sulfonyl chloride 116.3 was synthesized from intermediate 116.2 following the procedure described in Method AB. The crude sulfonyl chloride was used immediately in the forwarding step without further purification.

Synthesis of tert-butyl (R)-5-(N-(2,2,2-trifluoro-1-(4-fluorophenyl)ethyl)sulfamoyl)-1H-pyrazolo[4,3-b]pyridine-1-carboxylate 116.4: Intermediate 116.4 was synthesized from sulfonyl chloride 116.3 following the protocol as described in Method AC using (R)-2,2,2-trifluoro-1-(4-fluorophenyl)ethan-1-amine hydrochloride. Crude compound was purified by column chromatography using 20% ethyl acetate in hexane as eluent to afford desire product 116.4 as yellow gum (90 mg, 11% yield). LCMS: m/z found 403.2 [M+H]⁺, rt=4.91 min (Method 31) [Xbridge C18 column (5 μm, 50×4.6 mm)].

Synthesis of tert-butyl (R)-5-(N-ethyl-N-(2,2,2-trifluoro-1-(4-fluorophenyl)ethyl)sulfamoyl)-1H-pyrazolo[4,3-b]pyridine-1-carboxylate 116.5: The ethylation of intermediate 116.4 was preformed following the protocol as described in Method AD. The crude residue was purified by column chromatography over silica gel using 20% ethyl acetate-hexane to obtain compound 116.5 as a sticky gum (45 mg, 50% yield). ¹H NMR (400 MHz, DMSO-d₆): δ 8.87 (s, 1H), 8.61 (d, J=8.0 Hz, 1H), 8.22 (d, J=8.7 Hz, 1H), 7.59-7.51 (m, 2H), 7.28-7.20 (m, 2H), 6.05-5.95 (m, 1H), 3.40-3.20 (m, 2H), 1.67 (s, 9H), 0.85 (t, J=7.1 Hz, 3H).

Synthesis of (R)—N-ethyl-N-(2,2,2-trifluoro-1-(4-fluorophenyl)ethyl)-1H-pyrazolo[4,3-b]pyridine-5-sulfonamide: N-Boc deprotection of intermediate 116.5 was performed following a method analogous to that described in Method AG. The volatiles were evaporated under reduced pressure and the crude was triturated with 50% diethyl ether-pentane. It was further lyophilized to obtain the compound Example 188 as a white solid (10 mg, 99.57% purity, 28% yield). LCMS: m/z found 403.18 [M+H]⁺, rt=2.97 min (Method 2) [Waters Acquity BEH C18 column (1.7 μm, 50×2.1 mm)]; ¹H NMR (400 MHz, DMSO-d₆): δ 14.10-13.67 (m, 1H), 8.57 (s, 1H), 8.27 (d, J=8.8 Hz, 1H), 7.98 (d, J=8.8 Hz, 1H), 7.57 (dd, J=5.3, 8.6 Hz, 2H), 7.21 (t, J=8.8 Hz, 2H), 5.99 (q, J=8.9 Hz, 1H), 3.39-3.33 (m, 2H), 0.85 (t, J=7.0 Hz, 3H).

### Example 189

### (R)—N-ethyl-N-(2,2,2-trifluoro-1-(4-fluorophenyl)ethyl)-1H-benzo[d][1,2,3]triazole-5-sulfonamide

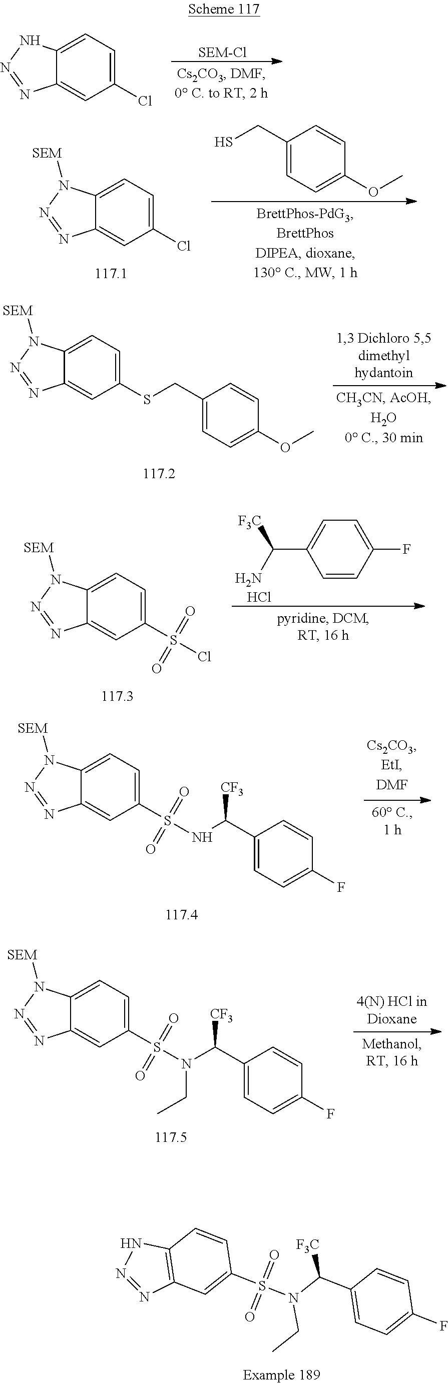

Synthesis of 5-chloro-1-((2-(trimethylsilyl)ethoxy)methyl)-1H-benzo[d][1,2,3]triazole 14.1: N-SEM protection: Method AJ1: 5-chloro-1H-benzo[d][1,2,3]triazole (500 mg, 3.26 mmol) was dissolved in DMF (10 mL) and cooled at 0° C. Cs₂CO₃ (2.13 g, 6.53 mmol) was added to it followed by SEM-Cl (817 mg, 4.90 mmol) and the reaction mixture was stirred at room temperature for 2 h. It was then diluted with water (30 mL) and extracted with ethyl acetate (2×25 mL). The combined organic layers were washed with water (2×25 mL) and brine (25 mL), dried over anhydrous Na₂SO₄, and the solvent was evaporated under reduced pressure. The crude residue was further purified by column chromatography over silica gel using 20% ethyl acetate in hexane to yield the compound 117.1 as a yellow gum (600 mg, 55% yield). LCMS: m/z found 284.0 [M+H]⁺, rt=2.08 min (Method 3) [YMC Triart C18 column (3 μm, 33×2.1 mm)].

Synthesis of 5-((4-methoxybenzyl)thio)-1-((2-(trimethylsilyl)ethoxy)methyl)-1H-benzo[d][1,2,3]triazole 117.2: Buchwald coupling: Method AA1: Intermediate 117.1 (500 mg, 1.76 mmol) was dissolved in dioxane (5 mL) and to this solution (4-methoxyphenyl)methanethiol (326 mg, 2.12 mmol) was added. The solution was degasified with argon and DIPEA (1 mL, 5.3 mmol) was added to it. Brettphos (32 mg, 0.03 mmol) was added to it under the inert atmosphere, followed by Brettphos Pd G3 (47 mg, 0.08 mmol). The reaction mixture was then heated at 130° C. for 1 h in a microwave oven. After completion of the reaction (monitored by TLC and LCMS) the residue was diluted with ethyl acetate (10 mL) and filtered through a sintered funnel. The filtrate was washed with water (20 mL) and brine (20 mL), dried over anhydrous Na₂SO₄ and the solvent was evaporated under reduced pressure. The crude was triturated with 5% ethyl acetate in hexane to provide the product 117.2 as a yellow gum (400 mg, 55% yield). LCMS: m/z found 402.1 [M+H]⁺, rt=2.21 min (Method 3) [YMC Triart C18 column (3 μm, 33×2.1 mm)].

Synthesis of 1-((2-(trimethylsilyl)ethoxy)methyl)-1H-benzo[d][1,2,3]triazole-5-sulfonyl chloride 117.3: Sulfonyl chloride 117.3 was synthesized from intermediate 117.2 following the procedure described in Method AB. The crude sulfonyl chloride was used immediately in the forwarding step without further purification.

Synthesis of (R)—N-(2,2,2-trifluoro-1-(4-fluorophenyl)ethyl)-1-((2-(trimethylsilyl)ethoxy)methyl)-1H-benzo[d][1,2,3]triazole-5-sulfonamide 117.4: Intermediate 117.4 was synthesized from sulfonyl chloride 17.3 following the protocol as described in Method AC using (R)-2,2,2-trifluoro-1-(4-fluorophenyl)ethan-1-amine hydrochloride. Crude compound was purified by column chromatography using 20% ethyl acetate in hexane as eluent to afford desire product 117.4 as yellow gum (90 mg, 15% yield). LCMS: m/z found 505.2 [M+H]⁺, rt=2.09 min (Method 3) [YMC Triart C18 column (3 μm, 33×2.1 mm)].

Synthesis of (R)—N-ethyl-N-(2,2,2-trifluoro-1-(4-fluorophenyl)ethyl)-1-((2-(trimethylsilyl)ethoxy)methyl)-1H-benzo[d][1,2,3]triazole-5-sulfonamide 117.5: The ethylation of intermediate 117.4 was performed following the protocol as described in Method AD. The crude was purified by column chromatography over silica gel using 20% ethyl acetate-hexane to obtain compound 117.5 as a sticky gum (45 mg, 47% yield). ¹H NMR (400 MHz, DMSO-d₆): δ 8.67 (s, 1H), 8.31 (d, J=8.6 Hz, 1H), 7.95 (d, J=8.8 Hz, 1H), 7.44 (t, J=6.9 Hz, 2H), 7.18 (t, J=8.6 Hz, 2H), 6.20 (S, 2H), 6.1-6.0 (m, 1H), 3.56 (t, J=7.6 Hz, 2H), 3.45-3.20 (m, 2H), 0.94 (t, J=7.0 Hz, 3H), 0.80 (t, J=7.8 Hz, 2H), −0.14 (s, 9H).

Synthesis of (R)—N-ethyl-N-(2,2,2-trifluoro-1-(4-fluorophenyl)ethyl)-1H-benzo[d][1,2,3]triazole-5-sulfonamide: N-SEM deprotection of intermediate 117.5 was performed following a method analogous to that described in Method AK. The volatiles were evaporated under reduced pressure and the crude was triturated with 50% diethyl ether-pentane. It was further lyophilized to obtain the compound Example 189 as a white solid (15 mg, 99.48% purity, 45% yield). LCMS: m/z found 403.18 [M+H]⁺, rt=2.96 min (Method 2) [Waters Acquity BEH C8 column (1.7 μm, 50×2.1 mm)]; ¹H NMR (400 MHz, DMSO-d₆) δ 8.58 (s, 1H), 8.03 (d, J=8.8 Hz, 1H), 7.89 (dd, J=1.8, 8.7 Hz, 1H), 7.44 (dd, J=5.2, 8.6 Hz, 2H), 7.19 (t, J=8.8 Hz, 2H), 6.09 (q, J=8.6 Hz, 1H), 3.45-3.34 (m, 1H), 3.28-3.13 (m, 2H), 0.92 (t, J=7.0 Hz, 3H).

### Example 190

### (R)—N,1-diethyl-N-(2,2,2-trifluoro-1-(4-fluorophenyl)ethyl)-1H-pyrazolo[3,4-b]pyridine-3-sulfonamide

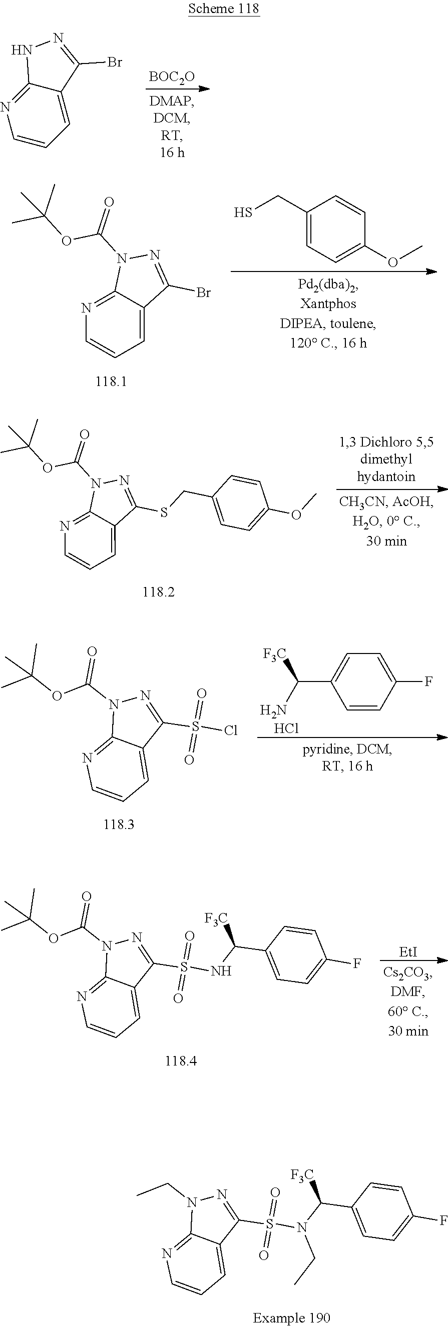

Synthesis of tert-butyl 3-bromo-1H-pyrazolo[3,4-b]pyridine-1-carboxylate 118.1: Intermediate 118.1 was synthesized from 3-bromo-1H-pyrazolo[3,4-b]pyridine following a method analogous to that described in Method AF. The crude residue was purified by column chromatography over silica gel using 20% ethyl acetate-hexane to obtain the compound 118.1 as a colorless gum (2.2 g, 73% yield). ¹H NMR (400 MHz, DMSO-d₆): δ 8.78 (d, J=4 Hz, 1H), 8.22 (d, J=8 Hz, 1H), 7.54-7.51 (m, 1H), 1.63 (s, 9H).

Synthesis of tert-butyl 3-((4-methoxybenzyl)thio)-1H-pyrazolo[3,4-b]pyridine-1-carboxylate 11.2: Intermediate 118.2 was synthesized from intermediate 118.1 following a method analogous to that described in Method AA using (4-methoxyphenyl)methanethiol. The crude residue was purified by column chromatography over silica gel using 30% ethyl acetate-hexane to yield compound 118.2 as a light-yellow gum (1.0 g, 80% yield). ¹H NMR (400 MHz, DMSO-d₆): δ 8.71-8.70 (m, 1H), δ 8.18-8.16 (m, 1H), δ 7.42-7.38 (m, 3H), 6.86 (d, J=12 Hz), 4.45 (s, 2H), 3.71 (s, 3H), 1.64 (s, 9H).

Synthesis of tert-butyl 3-(chlorosulfonyl)-1H-pyrazolo[3,4-b]pyridine-1-carboxylate 118.3: Sulfonyl chloride 118.3 was synthesized from intermediate 118.2 following the procedure described in Method BB. The crude sulfonyl chloride was used immediately in the forwarding step without further purification.

Synthesis of tert-butyl (R)-3-(N-(2,2,2-trifluoro-1-(4-fluorophenyl)ethyl)sulfamoyl)-1H-pyrazolo[3,4-b]pyridine-1-carboxylate 118.4: Intermediate 118.4 was synthesized from sulfonyl chloride 118.3 following the protocol as described in Method AC using (R)-2,2,2-trifluoro-1-(4-fluorophenyl)ethan-1-amine hydrochloride. The compound was purified by column chromatography over silica gel using 40% ethyl acetate in hexane to isolate desired compound 118.4 as off white solid (140 mg, 25% yield). LCMS: m/z found 475.2 [M+H]⁺, rt=3.34 min (Method 4) [Waters Xbridge C18 column (5 μm, 50×4.6 mm)].

Synthesis of (R)—N,1-diethyl-N-(2,2,2-trifluoro-1-(4-fluorophenyl)ethyl)-1H-pyrazolo[3,4-b]pyridine-3-sulfonamide: The ethylation of intermediate 118.4 was preformed following the protocol as described in Method AD. The crude was purified by column chromatography over silica gel using 30% ethyl acetate-hexane to obtain compound Example 190 as a colorless gum (20 mg, 23% yield). ¹H NMR (400 MHz, DMSO-d₆): δ 8.72 (d, J=4.4 Hz, 1H), 8.40 (d, J=8.1 Hz, 1H), 7.52-7.44 (m, 1H), 7.40 (dd, J=5.3, 8.5 Hz, 2H), 7.14 (t, J=8.7 Hz, 2H), 5.99 (q, J=8.7 Hz, 1H), 4.59 (q, J=7.3 Hz, 2H), 3.51-3.32 (m, 2H), 1.42 (t, J=7.2 Hz, 3H), 0.99 (t, J=7.0 Hz, 3H).

### Example 191

### (R)—N-(1-(4-chlorophenyl)-2,2,2-trifluoroethyl)-N-methyl-[1,2,4]triazolo[1,5-a]pyridine-7-sulfonamide

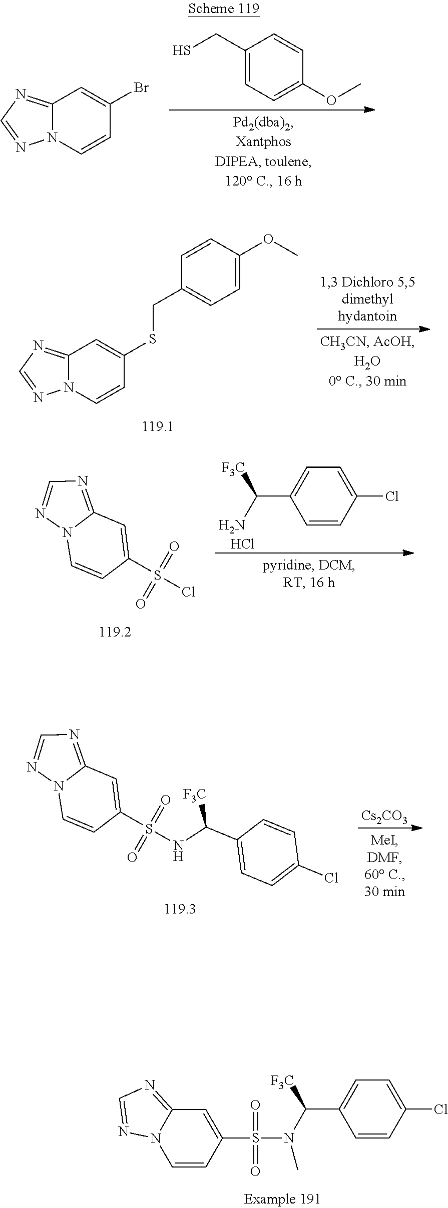

Synthesis of 7-((4-methoxybenzyl)thio)-[1,2,4]triazolo[1,5-a]pyridine 119.1: Intermediate 119.1 was synthesized from 7-bromo-[1,2,4]triazolo[1,5-a]pyridine following a method analogous to that described in Method AA using (4-methoxyphenyl)methanethiol. The crude was purified by column chromatography over silica gel using 30% ethyl acetate-hexane to yield compound 119.1 as a light yellow gum (450 mg, 66% yield). LCMS: m/z found 272.1 [M+H]⁺, rt=1.74 min (Method 3) [YMC Triart C18 column (3 μm, 33×2.1 mm)].

Synthesis of [1,2,4]triazolo[1,5-a]pyridine-7-sulfonyl chloride 119.2: Sulfonyl chloride 119.2 was synthesized from Intermediate 119.1 following the procedure described in Method AB. The crude sulfonyl chloride was used immediately in the forwarding step without further purification.

Synthesis of (R)—N-(1-(4-chlorophenyl)-2,2,2-trifluoroethyl)-[1,2,4]triazolo[1,5-a]pyridine-7-sulfonamide 119.3: Intermediate 11.3 was synthesized from sulfonyl chloride 119.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 119.3 as a yellow gum (110 mg, 17% yield). ¹H NMR (400 MHz, DMSO-d₆): δ 7.78 (d, J=7.9 Hz, 1H), 9.03 (d, J=7.3 Hz, 1H), 8.68 (s, 1H), 8.06 (s, 1H), 7.41 (d, J=7.8 Hz, 2H), 7.32-7.15 (m, 3H), 5.55-5.45 (m, 1H).

Synthesis of (R)—N-(1-(4-chlorophenyl)-2,2,2-trifluoroethyl)-N-methyl-[1,2,4]triazolo[1,5-a]pyridine-7-sulfonamide: The methylation of intermediate 119.3 was preformed following the protocol as described in Method AD. The crude was purified by column chromatography over silica gel using 40% ethyl acetate-hexane to obtain compound Example 191 as a colorless gum (55 mg, 48% yield). LCMS: m/z found 405.13 [M+H]⁺, rt=2.98 min (Method 2) [Waters Acquity BEH C8 column (1.7 μm, 50×2.1 mm)]; ¹H NMR (400 MHz, DMSO-d₆): δ 9.21 (d, J=7.2 Hz, 1H), 8.77 (s, 1H), 8.47 (s, 1H), 7.59 (dd, J=2.0, 7.2 Hz, 1H), 7.44 (s, 4H), 6.17 (q, J=8.6 Hz, 1H), 2.84 (s, 3H).

### Example 192

### (R)—N-ethyl-N-(2,2,2-trifluoro-1-(4-fluorophenyl)ethyl)-[1,2,4]triazolo[4,3-b]pyridazine-6-sulfonamide

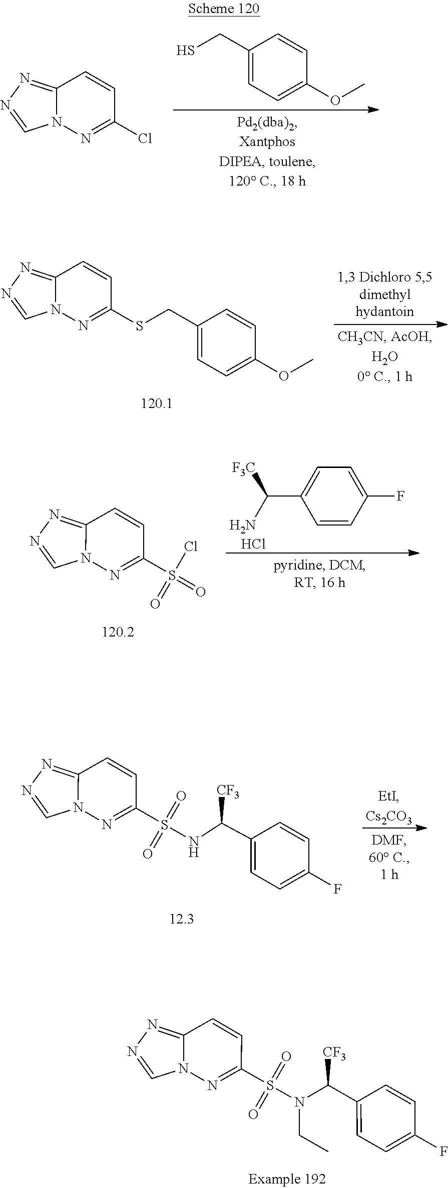

Synthesis of 6-((4-methoxybenzyl)thio)-[1,2,4]triazolo[4,3-b]pyridazine 120.1: Intermediate 120.1 was synthesized from of 6-chloro-[1,2,4]triazolo[4,3-b]pyridazine following a method analogous to that described in Method AA using (4-methoxyphenyl)methanethiol. The crude residue was purified by column chromatography over silica gel using 1:4 ethyl acetate-hexane to obtain compound 120.1 as a yellow sticky solid (4.5 g, 73% yield). ¹H NMR (400 MHz, DMSO-d₆): δ 9.58 (s, 1H), 8.23-8.15 (m, 1H), 7.42 (d, J=8.5 Hz, 2H), 7.25 (d, J=9.7 Hz, 1H), 6.89 (d, J=8.6 Hz, 2H), 4.41 (s, 2H), 3.72 (s, 3H).

Synthesis of [1,2,4]triazolo[4,3-b]pyridazine-6-sulfonyl chloride 120.2: Sulfonyl chloride 120.2 was synthesized from Intermediate 12.1 following the procedure described in Method AB. The crude sulfonyl chloride was used immediately in the forwarding step without further purification.

Synthesis of (R)—N-(2,2,2-trifluoro-1-(4-fluorophenyl)ethyl)-[1,2,4]triazolo[4,3-b]pyridazine-6-sulfonamide 120.3: Intermediate 120.3 was synthesized from sulfonyl chloride 120.2 following the protocol as described in Method AC using (R)-2,2,2-trifluoro-1-(4-fluorophenyl)ethan-1-amine hydrochloride. The crude was purified by column chromatography over silica gel using 35% ethyl acetate in hexane to isolate the sulfonamide 120.3 as a yellow sticky gum (35 mg, 8% yield). LCMS: m/z found 375.9 (M+H)⁺, rt=2.52 min (Method 4) [Waters Xbridge C18 column (5 μm, 50×4.6 mm)].

Synthesis of (R)—N-ethyl-N-(2,2,2-trifluoro-1-(4-fluorophenyl)ethyl)-[1,2,4]triazolo[4,3-b]pyridazine-6-sulfonamide: The ethylation of intermediate 120.3 was preformed following the protocol as described in Method AD. The crude was purified by column chromatography over silica gel using 1:4 ethyl acetate-hexane to obtain compound Example 192 as a colorless oil (8 mg, 21% yield, 99.23% purity). LCMS: m/z found 404.0 [M+H]⁺, rt=2.62 min (Method 9) [Waters Xbridge C18 column (3.5 μm, 50×3 mm)]. ¹H NMR (400 MHz, Chloroform-d): δ 9.17 (s, 1H), 8.33 (d, J=9.3 Hz, 1H), 7.66 (d, J=9.6 Hz, 1H), 7.57-7.49 (m, 2H), 7.13 (t, J=8.6 Hz, 2H), 5.76 (q, J=8.3 Hz, 1H), 3.60-3.46 (m, 2H), 0.99 (t, J=7.0 Hz, 3H).

### Example 193

### (R)—N-(1-(4-chlorophenyl)-2,2,2-trifluoroethyl)-N-methyl-3H-imidazo[4,5-b]pyridine-6-sulfonamide

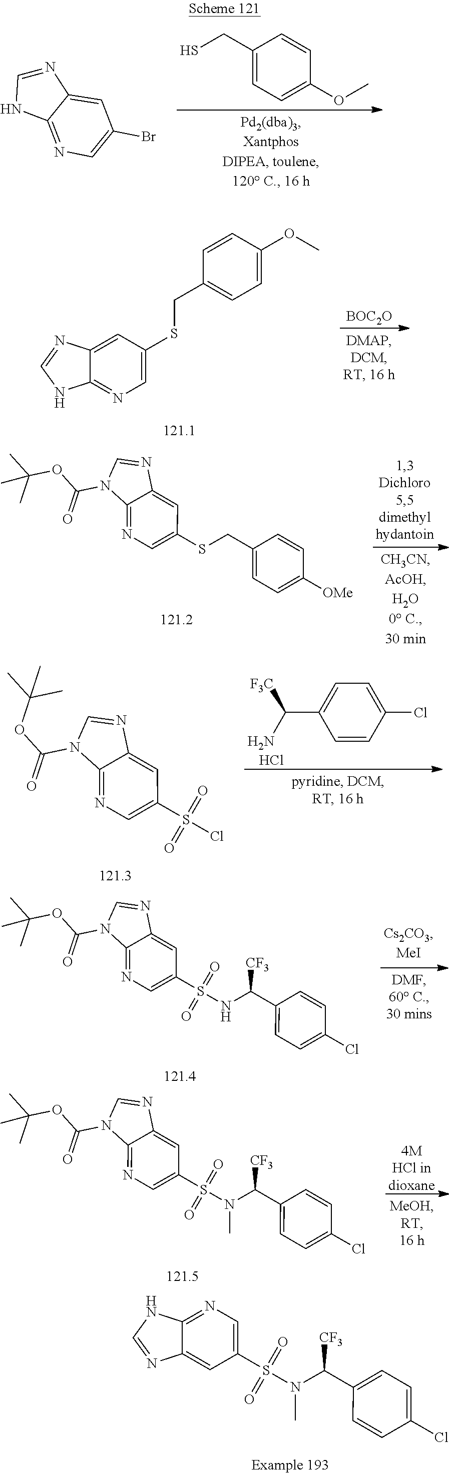

Synthesis of 6-((4-methoxybenzyl)thio)-3H-imidazo[4,5-b]pyridine 121.1: Intermediate 121.1 was synthesized from 6-bromo-3H-imidazo[4,5-b]pyridine following a method analogous to that described in Method AA using (4-methoxyphenyl)methanethiol. The crude residue was purified by column chromatography over silica gel using 30% ethyl acetate-hexane to yield compound 121.1 as a yellow gum (1.0 g, 49% yield). LCMS: m/z found 398.1 [M+H]⁺, rt=1.98 min (Method 3) [YMC Triart C18 column (3 μm, 33×2.1 mm)].

Synthesis of tert-butyl 6-((4-methoxybenzyl)thio)-3H-imidazo[4,5-b]pyridine-3-carboxylate 121.2: Intermediate 121.2 was synthesized from intermediate 121.1 following a method analogous to that described in Method AF. The crude was purified by column chromatography over silica gel using 20% ethyl acetate-hexane to obtain the compound 121.2 as a colorless gum (600 mg, 44% yield). LCMS: m/z found 372.1 [M+H]⁺, rt=3.66 min (Method 4) [Waters Xbridge C18 column (5 μm, 50×4.6 mm)].

Synthesis of tert-butyl 6-(chlorosulfonyl)-3H-imidazo[4,5-b]pyridine-3-carboxylate 121.3: Sulfonyl chloride 121.3 was synthesized from intermediate 121.2 following the procedure described in Method AB. The crude sulfonyl chloride was used immediately in the forwarding step without further purification.

Synthesis of tert-butyl (R)-6-(N-(1-(4-chlorophenyl)-2,2,2-trifluoroethyl)sulfamoyl)-3H-imidazo[4,5-b]pyridine-3-carboxylate 121.4: Intermediate 121.4 was synthesized from sulfonyl chloride 121.3 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 121.4 as a yellow gum (110 mg, 11% yield). LCMS: m/z found 491.1 [M+H]⁺, rt=1.90 min (Method 8) [Waters Xbridge C18 column (3.5 μm, 50×3 mm)].

Synthesis of tert-butyl (R)-6-(N-(1-(4-chlorophenyl)-2,2,2-trifluoroethyl)-N-methylsulfamoyl)-3H-imidazo[4,5-b]pyridine-3-carboxylate 121.5: The methylation of intermediate 121.5 was preformed following the protocol as described in Method AD. The crude residue was purified by column chromatography over silica gel using 30% ethyl acetate-hexane to obtain compound 121.5 as a colorless gum (20 mg, 20% yield). LCMS: m/z found 505.1 [M+H]⁺, rt=2.09 min (Method 8) [Waters Xbridge C18 column (3.5 μm, 50×3 mm)].

Synthesis of (R)—N-(1-(4-chlorophenyl)-2,2,2-trifluoroethyl)-N-methyl-3H-imidazo[4,5-b]pyridine-6-sulfonamide: N-Boc deprotection of intermediate 121.5 was performed following a method analogous to that described in Method AG. The volatiles were evaporated under reduced pressure and the crude was triturated with 50% diethyl ether-pentane. It was further lyophilized to obtain the compound Example 193 as an off-white solid (7 mg, 99.64% purity, 20% yield). LCMS: m/z found 405.13 [M+H]⁺, rt=2.88 min (Method 2) [Waters Acquity BEH C8 column (1.7 μm, 50×2.1 mm)]; ¹H NMR (400 MHz, DMSO-d₆) δ 13.85-13.25 (m, 1H), 8.84 (d, J=1.9 Hz, 1H), 8.71 (s, 1H), 8.52 (s, 1H), 7.46-7.35 (m, 4H), 6.14 (q, J=8.3 Hz, 1H), 2.77 (s, 3H).

### Example 194

### (R)—N-(1-(4-chlorophenyl)-2,2,2-trifluoroethyl)-N-methylimidazo[1,2-a]pyridine-7-sulfonamide

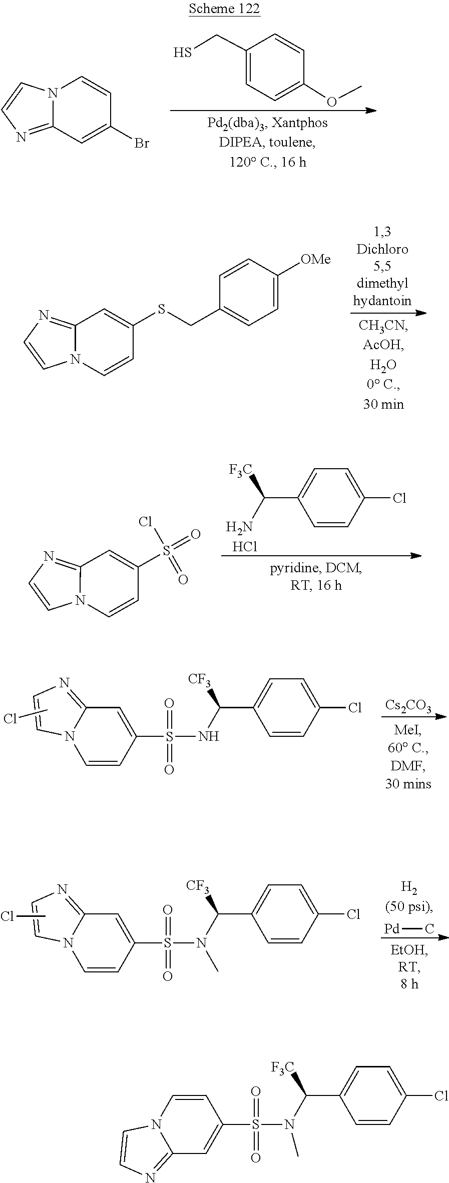

Synthesis of 7-((4-methoxybenzyl)thio)imidazo[1,2-a]pyridine 122.1: Intermediate 122.1 was synthesized from 7-bromoimidazo[1,2-a]pyridine following a method analogous to that described in Method AA using (4-methoxyphenyl)methanethiol. The crude residue was purified by column chromatography over silica gel using 20% ethyl acetate-hexane to yield compound 122.1 as a yellow gum (1.9 g, 67% yield). LCMS: m/z found 271.0 [M+H]⁺, rt=1.62 min (Method 30) [Waters Acquity BEH C18 column (1.7 μm, 50×2.1 mm)].

Synthesis of imidazo[1,2-a]pyridine-7-sulfonyl chloride 122.2: Sulfonyl chloride 122.2 was synthesized from intermediate 122.1 following the procedure described in Method AB. The crude sulfonyl chloride was used immediately in the forwarding step without further purification.

Synthesis of (R)-chloro-N-(1-(4-chlorophenyl)-2,2,2-trifluoroethyl)imidazo[1,2-a]pyridine-7-sulfonamide 122.3: Sulfonamide 122.3 was synthesized from sulfonyl chloride 122.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 122.3 as a light yellow gum (230 mg, 12% yield). LCMS: m/z found 424.2 [M+H]⁺, rt=2.2 min (Method 12) [YMC Triart C18 column (3 μm, 33×2.1 mm)].

Synthesis of (R)-chloro-N-(1-(4-chlorophenyl)-2,2,2-trifluoroethyl)-N-methylimidazo[1,2-a]pyridine-7-sulfonamide 122.4: The methylation of intermediate 122.3 was performed following the protocol as described in Method AD. The crude residue was purified by column chromatography over silica gel using 20% ethyl acetate-hexane to obtain compound 122.4 as a colorless gum (130 mg, 66% yield). LCMS: m/z found 438.3 [M+H]⁺, rt=2.12 min (Method 30) [Waters Acquity BEH C18 column (1.7 μm, 50×2.1 mm)].

Synthesis of (R)—N-(1-(4-chlorophenyl)-2,2,2-trifluoroethyl)-N-methylimidazo[1,2-a]pyridine-7-sulfonamide: The hydrogenation of intermediate 122.4 was preformed following the protocol as described in Method AE. The crude was purified by column chromatography over silica gel using 40% ethyl acetate in hexane to obtain compound Example 194 as a white solid (25 mg, 99.42% purity, 20% yield). LCMS: m/z found 404.15 [M+H]1, rt=2.90 min (Method 2) [Waters Acquity BEH C18 column (1.7 μm, 50×2.1 mm)]; ¹H NMR (400 MHz, Methanol-d₄): δ 8.61 (d, J=7.1 Hz, 1H), 8.12 (s, 1H), 8.05 (s, 1H), 7.81 (s, 1H), 7.47-7.35 (m, 4H), 7.25 (dd, J=2.0, 7.2 Hz, 1H), 5.99 (q, J=8.3 Hz, 1H), 2.83 (s, 3H).

### Example 195 to Example 197

### (R)—N-(1-(4-chlorophenyl)-2,2,2-trifluoroethyl)-N-methylimidazo[1,2-a]pyrazine-2-sulfonamide (Example 195); (R)—N-methyl-N-(2,2,2-trifluoro-1-(4-methoxyphenyl)ethyl)imidazo[1,2-a]pyrazine-2-sulfonamide (Example 196); (R)—N-methyl-N-(2,2,2-trifluoro-1-(p-tolyl)ethyl)imidazo[1,2-a]pyrazine-2-sulfonamide (Example 197)

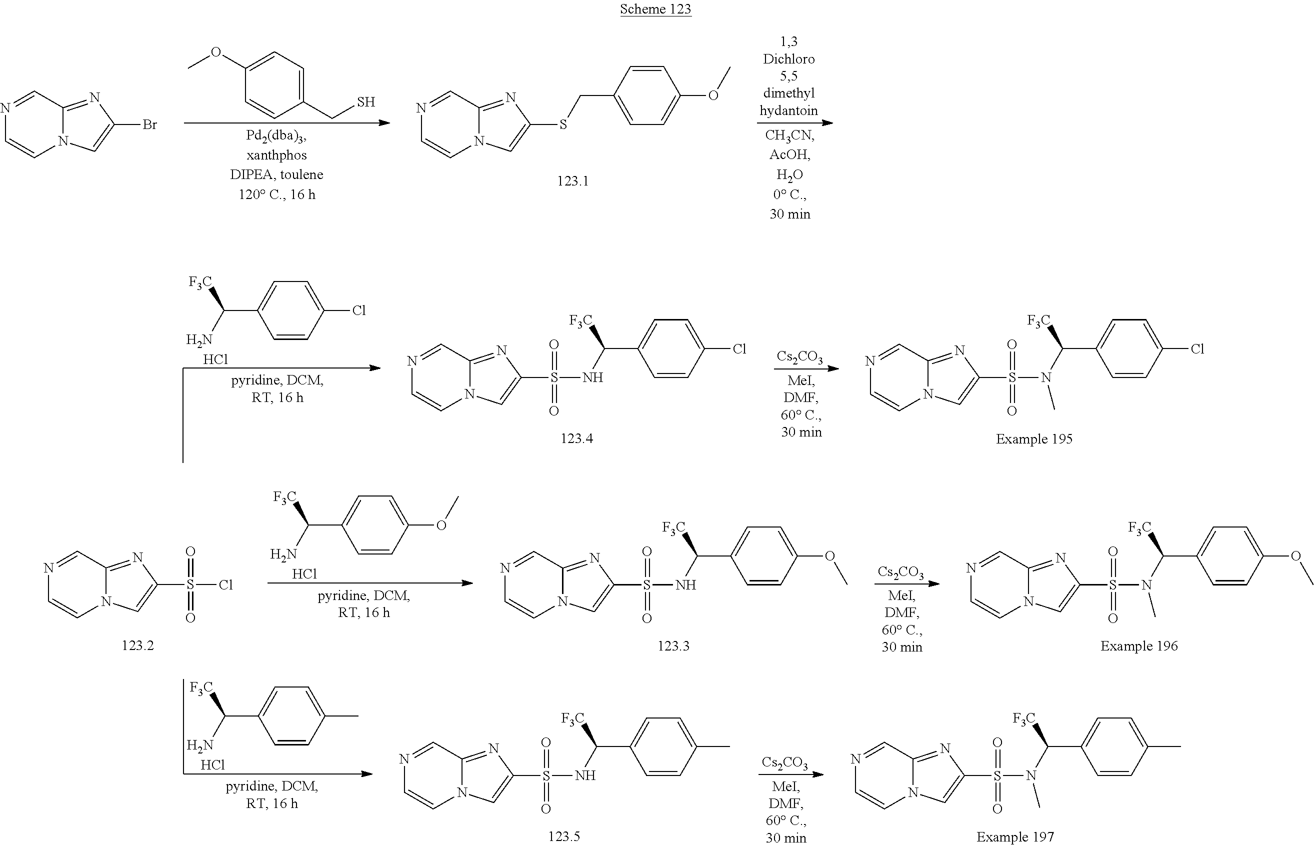

Synthesis of 2-((4-methoxybenzyl)thio)imidazo[1,2-a]pyrazine 123.1: Intermediate 123.1 was synthesized from 2-bromoimidazo[1,2-a]pyrazine following a method analogous to that described in Method AA using (4-methoxyphenyl)methanethiol. The crude residue was purified by column chromatography over silica gel using 50% ethyl acetate-hexane to yield compound 123.1 as a yellow gum (900 mg, 65% yield). LCMS: m/z found 272.0 [M+H]⁺, rt=1.68 min (Method 3) [YMC Triart C18 column (3 μm, 33×2.1 mm)].

Synthesis of imidazo[1,2-a]pyrazine-2-sulfonyl chloride 123.2: Sulfonyl chloride 123.2 was synthesized from intermediate 123.2 following the procedure described in Method AB. The crude sulfonyl chloride was used immediately in the forwarding step without further purification.

Synthesis of (R)—N-(1-(4-chlorophenyl)-2,2,2-trifluoroethyl)imidazo[1,2-a]pyrazine-2-sulfonamide 123.3: Sulfonamide 123.3 was synthesized from sulfonyl chloride 123.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 123.3 as yellow gum (70 mg, 15% yield). LCMS: m/z found 387.0 [M+H]⁺, rt=1.67 min (Method 3) [YMC Triart C18 column (3 μm, 33×2.1 mm)].

Synthesis of (R)—N-(1-(4-chlorophenyl)-2,2,2-trifluoroethyl)-N-methylimidazo[1,2-a]pyrazine-2-sulfonamide: The methylation of intermediate 123.3 was preformed following the protocol as described in Method AD. The crude residue was purified by column chromatography over silica gel using 25% ethyl acetate-hexane to obtain compound Example 195 as a light-yellow sticky gum (13 mg, 20% yield). LCMS: m/z found 405.12 [M+H]⁺, rt=3.03 min (Method 2) [Waters Acquity BEH C8 column (1.7 μm, 50×2.1 mm)]; ¹H NMR (400 MHz, DMSO-d₆): δ 9.34 (d, J=1.6 Hz, 1H), 8.83-8.77 (m, 1H), 8.49 (s, 1H), 8.22 (d, J=4.4 Hz, 1H), 7.45 (d, J=8.8 Hz, 2H), 7.40 (d, J=8.4 Hz, 2H), 6.21 (q, J=8.6 Hz, 1H), 2.86 (s, 3H).

Synthesis of (R)—N-methyl-N-(2,2,2-trifluoro-1-(4-methoxyphenyl)ethyl)imidazo[1,2-a]pyrazine-2-sulfonamide 123.4: Sulfonamide 123.4 was synthesized from sulfonyl chloride 123.2 following the protocol as described in Method AC using (R)-2,2,2-trifluoro-1-(4-methoxyphenyl)ethan-1-amine hydrochloride. The compound was purified by column chromatography over silica gel using 40% ethyl acetate in hexane to isolate desired compound 123.4 as yellow gum (65 mg, 13% yield). LCMS: m/z found 391.1 [M+H]⁺, rt=1.57 min (Method 8) [Waters Xbridge C18 column (3.5 μm, 50×3 mm)].

Synthesis of (R)—N-methyl-N-(2,2,2-trifluoro-1-(4-methoxyphenyl)ethyl)imidazo[1,2-a]pyrazine-2-sulfonamide: The methylation of intermediate 123.4 was preformed following the protocol as described in Method AD. The crude residue was purified by column chromatography over silica gel using 25% ethyl acetate-hexane to obtain compound Example 196 as a light-yellow sticky gum (13 mg, 99.95% purity, 20% yield). LCMS: m/z found 401.27 [M+H]⁺, rt=2.92 min (Method 2) [Waters Acquity BEH C8 column (1.7 μm, 50×2.1 mm)]. ¹H NMR (400 MHz, DMSO-d₆): δ 9.20 (s, 1H), 8.69 (s, 1H), 8.62 (dd, J=1.7, 4.6 Hz, 1H), 8.06 (d, J=4.6 Hz, 1H), 7.26 (d, J=8.5 Hz, 2H), 6.83 (d, J=8.8 Hz, 2H), 5.78 (q, J=8.7 Hz, 1H), 3.69 (s, 3H), 2.79 (s, 3H).

Synthesis of (R)—N-(2,2,2-trifluoro-1-(p-tolyl)ethyl)imidazo[1,2-a]pyrazine-2-sulfonamide 123.5: Sulfonamide 123.5 was synthesized from sulfonyl chloride 123.2 following the protocol as described in Method AC using (R)-2,2,2-trifluoro-1-(p-tolyl)ethan-1-amine hydrochloride. The compound was purified by column chromatography over silica gel using 40% ethyl acetate in hexane to isolate desired compound 123.5 as a yellow gum (70 mg, 17% yield). LCMS: m/z found 371.0 [M+H]⁺, rt=1.70 min (Method 3) [YMC Triart C18 column (3 μm, 33×2.1 mm)].

Synthesis of (R)—N-methyl-N-(2,2,2-trifluoro-1-(p-tolyl)ethyl)imidazo[1,2-a]pyrazine-2-sulfonamide: The methylation of intermediate 123.5 was performed following the protocol as described in Method AD. The crude was purified by column chromatography over silica gel using 30% ethyl acetate-hexane to obtain compound Example 197 as a light-yellow sticky gum (31 mg, 97.67% purity, 44% yield). LCMS: m/z found 385.18[M+H]⁺, rt=3.01 min (Method 2) [Waters Acquity BEH C8 column (1.7 μm, 50×2.1 mm)]; ¹H NMR (400 MHz, DMSO-d₆): δ 9.20 (s, 1H), 8.69 (s, 1H), 8.65-8.59 (m, 1H), 8.06 (d, J=4.8 Hz, 1H), 7.27 (d, J=8.4 Hz, 2H), 6.83 (d, J=8.4 Hz, 2H), 5.86-5.69 (m, 1H), 3.70 (s, 3H), 2.80 (s, 3H).

### Example 198 and Example 199

### (R)—N-ethyl-N-(2,2,2-trifluoro-1-(4-fluorophenyl)ethyl)imidazo[1,2-a]pyrazine-6-sulfonamide (Example 198) and (R)—N-(1-(4-chlorophenyl)-2,2,2-trifluoroethyl)-N-methylimidazo[1,2-a]pyrazine-6-sulfonamide (Example 199)

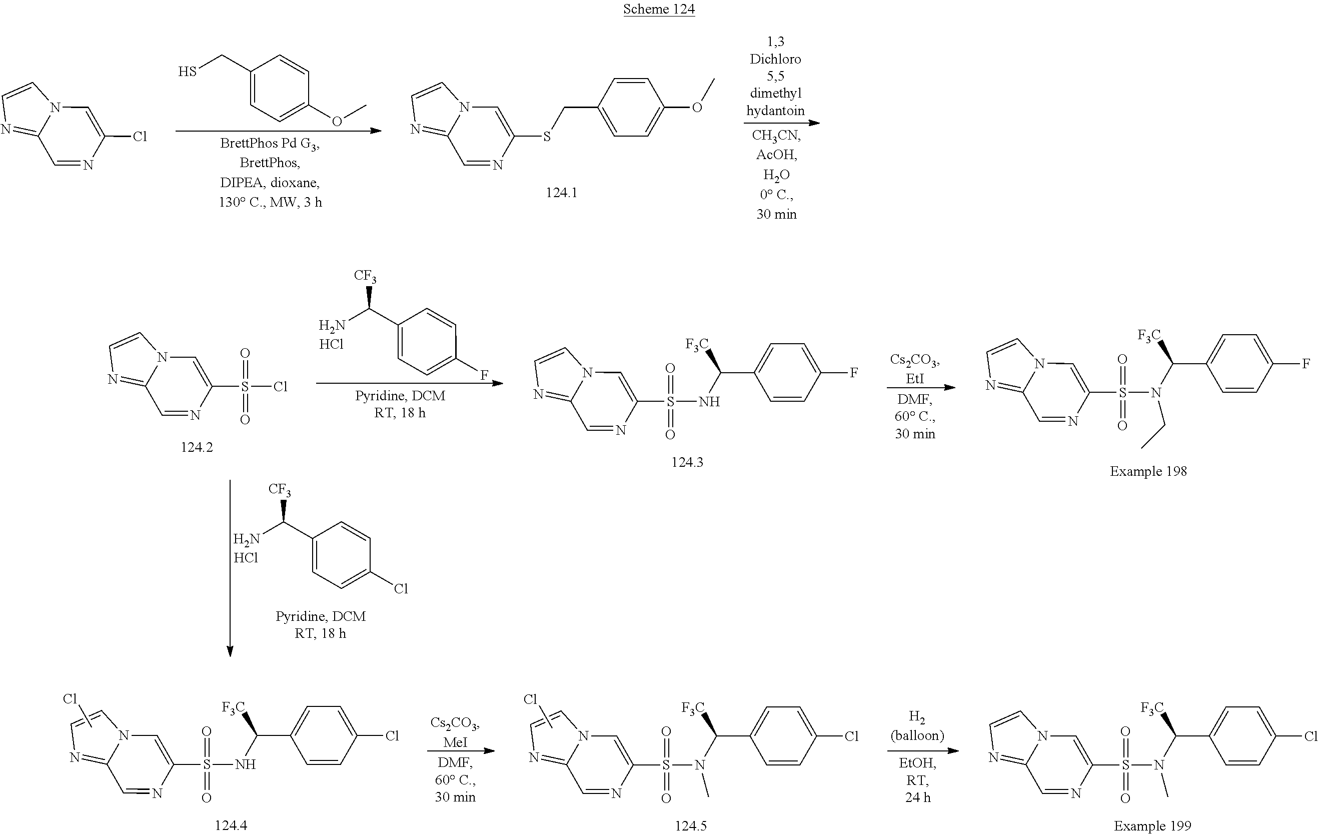

Synthesis of 6-((4-methoxybenzyl)thio)imidazo[1,2-a]pyrazine 124.1: Intermediate 124.1 was synthesized from 6-chloroimidazo[1,2-a]pyrazine following a method analogous to that described in Method AA1 using (4-methoxyphenyl)methanethiol. The crude residue was purified by column chromatography over silica gel using 20% ethyl acetate-hexane to yield compound 124.1 as a yellow gum (300 g (70% pure)). LCMS: m/z found 271.9 [M+H]⁺, rt=1.68 min (Method 3) [YMC Triart C18 column (3 μm, 33×2.1 mm)].

Synthesis of imidazo[1,2-a]pyrazine-6-sulfonyl chloride 124.2: Sulfonyl chloride 124.2 was synthesized from intermediate 124.1 following the procedure described in Method AB. The crude sulfonyl chloride was used immediately in the forwarding step without further purification.

Synthesis of (R)—N-(2,2,2-trifluoro-1-(4-fluorophenyl)ethyl)imidazo[1,2-a]pyrazine-6-sulfonamide 124.3: Sulfonamide 124.3 was synthesized from sulfonyl chloride 124.2 following the protocol as described in Method AC using (R)-2,2,2-trifluoro-1-(4-fluorophenyl)ethan-1-amine hydrochloride. The compound was purified by column chromatography over silica gel using 40% ethyl acetate in hexane to isolate desired compound 124.3 as yellow gum (110 mg, 70% pure). LCMS: m/z found 375.0 [M+H]⁺, rt=1.71 min (Method 3) [YMC Triart C18 column (3 μm, 33×2.1 mm)].

Synthesis of (R)—N-ethyl-N-(2,2,2-trifluoro-1-(4-fluorophenyl)ethyl)imidazo[1,2-a]pyrazine-6-sulfonamide: The ethylation of intermediate 124.3 was performed following the protocol as described in Method AD. The crude was purified by column chromatography over silica gel using 30% ethyl acetate-hexane to obtain compound Example 198 as a brown sticky gum (30 mg, 99.79% purity, 25% yield). LCMS: m/z found 403.15 [M+H]⁺, rt=3.12 min (Method 2) [Waters Acquity BEH C8 column (1.7 μm, 50×2.1 mm)]. ¹H NMR (400 MHz, Chloroform-d): δ 9.11 (s, 1H), 8.85 (s, 1H), 7.98 (s, 1H), 7.85 (s, 1H), 7.59 (t, J=7.8 Hz, 2H), 7.08 (t, J=8.6 Hz, 2H), 5.81 (q, J=8.1 Hz, 1H), 3.93-3.06 (m, 2H), 0.99 (t, J=7.0 Hz, 3H).

Synthesis of (R)—N-(1-(4-chlorophenyl)-2,2,2-trifluoroethyl)imidazo[1,2-a]pyrazine-6-sulfonamide 124.4: Sulfonamide 124.4 was synthesized from sulfonyl chloride 124.2 following the protocol as described in Method AC using (R)-2,2,2-trifluoro-1-(4-chlorophenyl)ethan-1-amine hydrochloride. The compound was purified by column chromatography over silica gel using 40% ethyl acetate in hexane to isolate desired compound 124.4 as a yellow gum (250 mg, 46% yield). LCMS: m/z found 424.9 [M+H]⁺, rt=1.84 min (Method 3) [YMC Triart C18 column (3 μm, 33×2.1 mm)].

Synthesis of 5- (and 6-)-chloro-(R)—N-(1-(4-chlorophenyl)-2,2,2-trifluoroethyl)-N-methylimidazo[1,2-a]pyrazine-6-sulfonamide 124.5: The methylation of intermediate 124.4 was preformed following the protocol as described in Method AD. The crude was purified by column chromatography over silica gel using 30% ethyl acetate-hexane to obtain compound 124.5 (mixture of 5- and 6-chloro substituted N-methylimidazo[1,2-a]pyrazine) as a colorless gum (250 mg, 60% yield). LCMS: m/z found 438.9 [M+H]⁺, rt=2.00 min (Method 3) [YMC Triart C18 column (3 μm, 33×2.1 mm)].

Synthesis of (R)—N-(1-(4-chlorophenyl)-2,2,2-trifluoroethyl)-N-methylimidazo[1,2-a]pyrazine-6-sulfonamide: The hydrogenation of intermediate 124.5 was preformed following the protocol as described in Method AE. The crude was purified by Reverse Phase Prep-HPLC to obtain compound Example 199 as a yellow solid (10 mg, 99.27% purity, 10% yield). RP method: Preparative HPLC was done in Waters BGM 2545 equipped with Waters PDA detector 2998 set to multiple-wavelength UV (200-400 nm) detection. Column name: YMC TRIART C18 (250×20 mm, 5μ) operating at ambient temperature and flow rate of 16 mL/min. Mobile phase: A=10 mM ammonium acetate in water, B=Acetonitrile. Gradient profile: Mobile phase initial composition of 70% A and 30% B, then to 45% A and 55% B in 3 min, then to 35% A and 65% B in 18 min, then to 0% A and 100% B in 19 min, held in this composition up to 22 min for column washing, then returned to initial composition in 22.5 min and held till 25 min. LCMS: m/z found 405.20 [M+H]⁺, rt=3.08 min (Method 2) [Waters Acquity BEH C8 column (1.7 μm, 50×2.1 mm)]. ¹H NMR (400 MHz, DMSO-d₆): δ 9.38 (s, 1H), 9.12 (s, 1H), 8.32 (s, 1H), 8.01 (s, 1H), 7.52-7.40 (m, 4H), 5.94 (q, J=8.6 Hz, 1H), 2.80 (s, 3H).

### Example 200

### (R)—N-(1-(4-chlorophenyl)-2,2,2-trifluoroethyl)-N-methylthiazolo[4,5-b]pyridine-6-sulfonamide

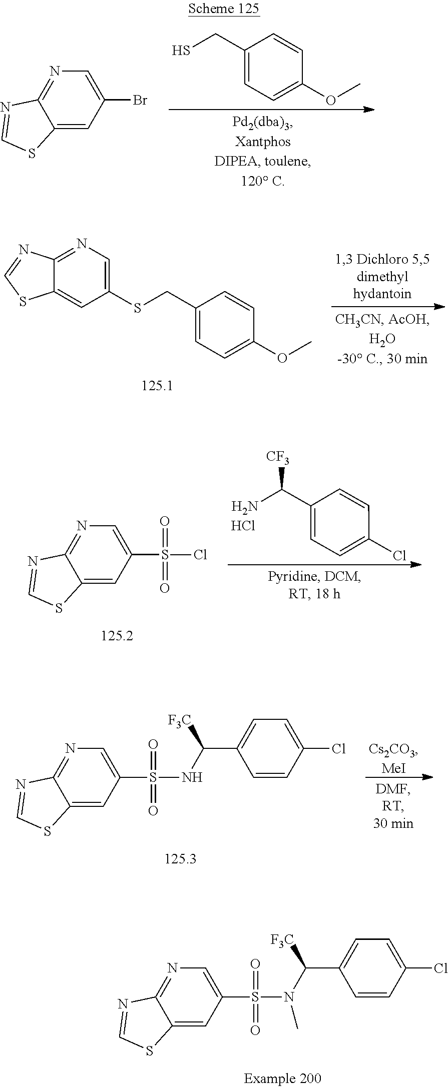

Synthesis of 6-((4-methoxybenzyl)thio)thiazolo[4,5-b]pyridine 125.1: Intermediate 125.1 was synthesized from of 6-bromothiazolo[4,5-b]pyridine following a method analogous to that described in Method AA using (4-methoxyphenyl)methanethiol. The crude residue was purified by column chromatography over silica gel using 1:4 ethyl acetate-hexane to obtain compound 125.1 as yellow sticky solid (750 mg, 56% yield). ¹H NMR (400 MHz, DMSO-d₆): δ 9.58 (s, 1H), 8.23-8.15 (m, 1H), 7.42 (d, J=8.5 Hz, 2H), 7.25 (d, J=9.7 Hz, 1H), 6.89 (d, J=8.6 Hz, 2H), 4.41 (s, 2H), 3.72 (s, 3H).

Synthesis of thiazolo[4,5-b]pyridine-6-sulfonyl chloride 125.2: Sulfonyl chloride 125.2 was synthesized from intermediate 125.1 following the procedure described in Method AB. The crude sulfonyl chloride was used immediately in the forwarding step without further purification.

Synthesis of (R)—N-(1-(4-chlorophenyl)-2,2,2-trifluoroethyl)thiazolo[4,5-b]pyridine-6-sulfonamide 125.3: Intermediate 125.3 was synthesized from sulfonyl chloride 125.2 following the protocol as described in Method AC using (R)-2,2,2-trifluoro-1-(4-chlorophenyl)ethan-1-amine hydrochloride. The crude was purified by column chromatography over silica gel using 35% ethyl acetate in hexane to isolate the sulfonamide 125.3 as yellow sticky gum (80 mg, 21% yield). LCMS: m/z found 375.9 (M+H)⁺, rt=2.52 min (Method 4) [Waters Xbridge C18 column (5 μm, 50×4.6 mm)].

Synthesis of (R)—N-(1-(4-chlorophenyl)-2,2,2-trifluoroethyl)-N-methylthiazolo[4,5-b]pyridine-6-sulfonamide: The ethylation of intermediate 125.3 was preformed following the protocol as described in Method AD. The crude was purified by column chromatography over silica gel using 1:4 ethyl acetate-hexane to obtain compound Example 200 as a colorless oil (20 mg, 97.17% purity, 24% yield). LCMS: m/z found 422.07 [M+H]⁺, rt=3.11 min (Method 2) [Waters Acquity BEH C8 column (1.7 μm, 50×2.1 mm)]. ¹H NMR (400 MHz, Chloroform-d): δ 9.17 (s, 1H), 8.33 (d, J=9.3 Hz, 1H), 7.66 (d, J=9.6 Hz, 1H), 7.57-7.49 (m, 2H), 7.13 (t, J=8.6 Hz, 2H), 5.76 (q, J=8.3 Hz, 1H), 3.60-3.46 (m, 2H), 0.99 (t, J=7.0 Hz, 3H).

### Example 201

### (R)—N-(1-(4-chlorophenyl)-2,2,2-trifluoroethyl)-N-methylpyrazolo[1,5-a]pyrimidine-6-sulfonamide

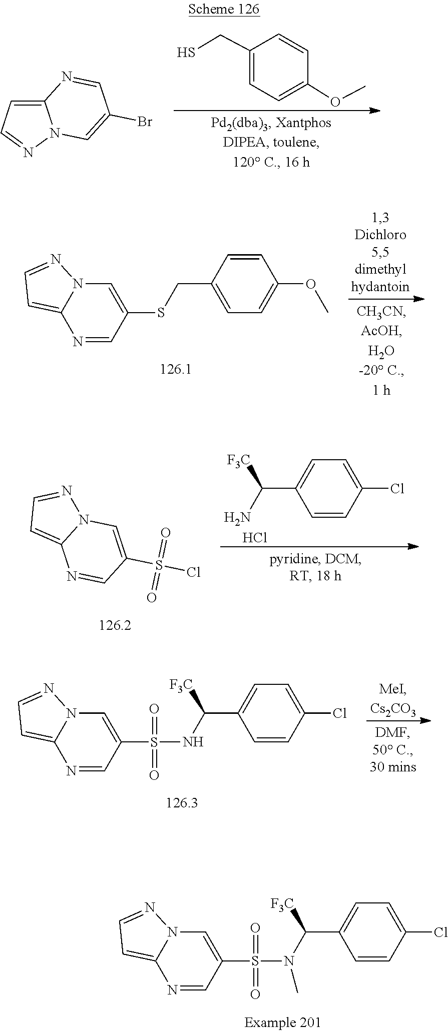

Synthesis of 6-((4-methoxybenzyl)thio)pyrazolo[1,5-a]pyrimidine 126.1: Intermediate 126.1 was synthesized from 6-bromopyrazolo[1,5-a]pyrimidine following a method analogous to that described in Method AA using (4-methoxyphenyl)methanethiol. The crude was purified by column chromatography over silica gel using 20% ethyl acetate-hexane to yield compound 126.1 as a yellow gum (600 mg, 84% yield). LCMS: m/z found 272.34 [M+H]⁺, rt=1.68 min (Method 2) [Waters Acquity BEH C18 column (1.7 μm, 50×2.1 mm).

Synthesis of pyrazolo[1,5-a]pyrimidine-6-sulfonyl chloride 126.2: Sulfonyl chloride 126.2 was synthesized from intermediate 126.1 following the procedure described in Method AB. The crude sulfonyl chloride was used immediately in the forwarding step without further purification.

Synthesis of (R)—N-(1-(4-chlorophenyl)-2,2,2-trifluoroethyl)pyrazolo[1,5-a]pyrimidine-6-sulfonamide 126.3: Sulfonamide 126.3 was synthesized from sulfonyl chloride 126.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 126.3 as a yellow gum (220 mg, 30% yield). LCMS: m/z found (391.0[M+H]⁺) rt=1.83 min (Method 3) [YMC Triart C18 column (3 μm, 33×2.1 mm)].

Synthesis of (R)—N-(1-(4-chlorophenyl)-2,2,2-trifluoroethyl)-N-methylpyrazolo[1,5-a]pyrimidine-6-sulfonamide: The methylation of intermediate 126.3 was preformed following the protocol as described in Method AD. The crude was purified by column chromatography over silica gel using 30% ethyl acetate-hexane to obtain compound Example 201 as an off white solid (40 mg, 98.94% purity, 17% yield). LCMS: m/z found 405.10 [M+H]⁺, rt=3.25 min (Method 2) [Waters Acquity BEH C8 column (1.7 μm, 50×2.1 mm)]. ¹H NMR (400 MHz, DMSO-d₆): δ 9.74 (d, J=1.0 Hz, 1H), 8.86 (d, J=1.9 Hz, 1H), 8.52 (d, J=2.0 Hz, 1H), 7.58-7.34 (m, 4H), 6.96 (d, J=1.3 Hz, 1H), 6.18 (q, J=8.4 Hz, 1H), 2.89 (s, 3H).

### Example 202

### (R)—N-(1-(4-chlorophenyl)-2,2,2-trifluoroethyl)-N-methyl-[1,2,3]triazolo[1,5-a]pyridine-5-sulfonamide

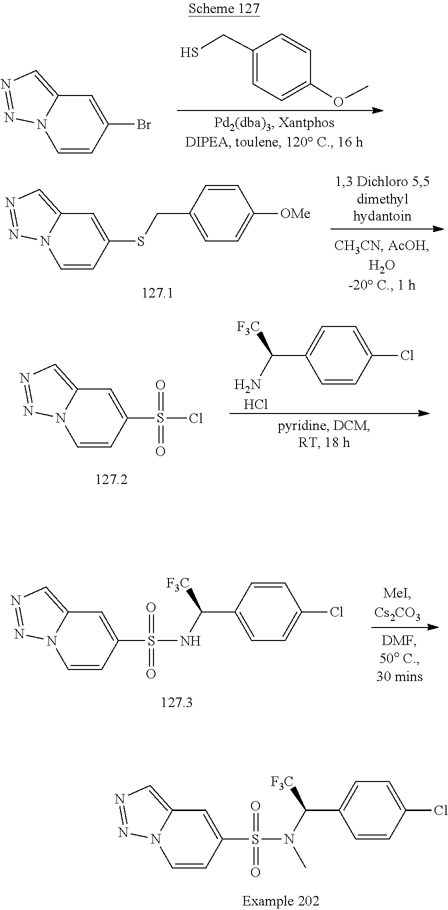

Synthesis of 5-((4-methoxybenzyl)thio)-[1,2,3]triazolo[1,5-a]pyridine 127.1: Intermediate 127.1 was synthesized from 5-bromo-[1,2,3]triazolo[1,5-a]pyridine following a method analogous to that described in Method AA using (4-methoxyphenyl)methanethiol. The crude residue was purified by column chromatography over silica gel using 20% ethyl acetate-hexane to yield compound 127.1 as a yellow gum (1.21 g, 84% yield). LCMS: m/z found 272.2 [M+H]⁺ rt=1.68 min (Method 8) [Waters Xbridge C18 column (3.5 μm, 50×3 mm).

Synthesis of [1,2,3]triazolo[1,5-a]pyridine-5-sulfonyl chloride 127.2: Sulfonyl chloride 127.2 was synthesized from intermediate 127.1 following the procedure described in Method AB. The crude sulfonyl chloride was used immediately in the forwarding step without further purification.

Synthesis of (R)—N-(1-(4-chlorophenyl)-2,2,2-trifluoroethyl)-[1,2,3]triazolo[1,5-a]pyridine-5-sulfonamide 127.3: Sulfonamide 127.3 was synthesized from sulfonyl chloride 127.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 127.3 as a yellow gum (180 mg, 20% yield). LCMS: m/z found 391.2 [M+H]⁺, rt=1.67 min (Method 3) [YMC Triart C18 column (3 μm, 33×2.1 mm).

Synthesis of (R)—N-(1-(4-chlorophenyl)-2,2,2-trifluoroethyl)-N-methyl-[1,2,3]triazolo[1,5-a]pyridine-5-sulfonamide: The methylation of intermediate 127.3 was preformed following the protocol as described in Method AD. The crude was purified by column chromatography over silica gel using 30% ethyl acetate-hexane to obtain compound Example 202 as an off-white solid (80 mg, 98.68% purity, 30% yield). LCMS: m/z found 405.10 [M+H]1, rt=3.09 min (Method 2) [Waters Acquity BEH C8 column (1.7 μm, 50×2.1 mm)]; ¹H NMR (400 MHz, DMSO-d₆): δ 9.27 (d, J=7.3 Hz, 1H), 8.66-8.61 (m, 1H), 8.50 (s, 1H), 7.52-7.40 (m, 5H), 6.12 (q, J=8.6 Hz, 1H), 2.80 (s, 3H).

### Example 203

### (R)—N-methyl-N-(2,2,2-trifluoro-1-(4-methoxyphenyl)ethyl)imidazo[1,2-a]pyridine-6-sulfonamide

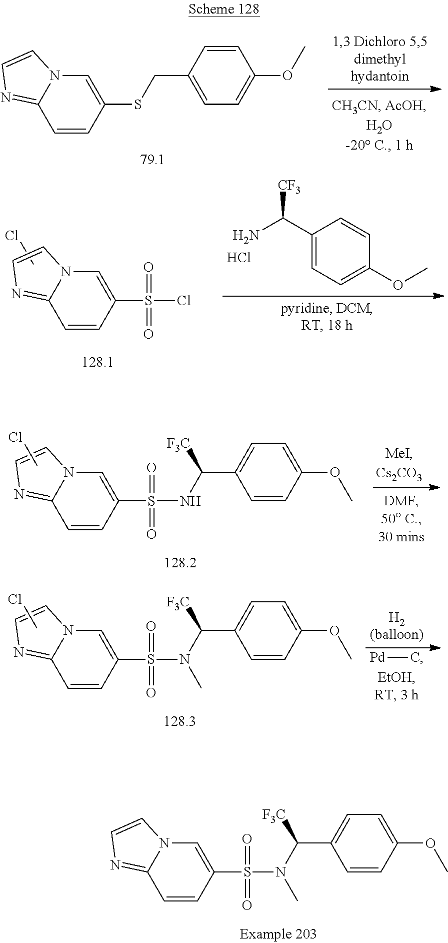

Synthesis of chloroimidazo[1,2-a]pyridine-6-sulfonyl chloride 128.1: Sulfonyl chloride 128.1 was synthesized from intermediate 79.1 following the procedure described in Method AB. The crude sulfonyl chloride was used immediately in the forwarding step without further purification.

Synthesis of chloro (R)—N-(2,2,2-trifluoro-1-(4-methoxyphenyl)ethyl)imidazo[1,2-a]pyridine-6-sulfonamide 128.2: Sulfonamide 128.2 was synthesized from sulfonyl chloride 128.1 following the protocol as described in Method AC using (R)-2,2,2-trifluoro-1-(4-methoxyphenyl)ethan-1-amine hydrochloride. The compound was purified by column chromatography over silica gel using 40% ethyl acetate in hexane to isolate desired compound 128.2 as a yellow gum (230 mg, 38% yield). LCMS: m/z found 420.0 [M+H]⁺, rt=1.82 min (Method 3) [YMC Triart C18 column (3 μm, 33×2.1 mm)].

Synthesis of chloro (R)—N-methyl-N-(2,2,2-trifluoro-1-(4-methoxyphenyl)ethyl)imidazo[1,2-a]pyridine-6-sulfonamide 128.3: The methylation of intermediate 128.2 was preformed following the protocol as described in Method AD. The crude was purified by column chromatography over silica gel using 30% ethyl acetate-hexane to obtain compound 128.3 as a colorless gum (120 mg, 50% yield). LCMS: m/z found 434.0 [M+H]⁺, rt=1.95 min (Method 3) [YMC Triart C18 column (3 μm, 33×2.1 mm)].

Synthesis of (R)—N-methyl-N-(2,2,2-trifluoro-1-(4-methoxyphenyl)ethyl)imidazo[1,2-a]pyridine-6-sulfonamide: The hydrogenation of intermediate 128.4 was performed as described here. Hydrogenation; Method E1: To a stirred solution of intermediate 128.3 (55 mg, 0.12 mmol) in ethanol (1 mL) Pd on C (10% w/w) (10 mg) was added and the reaction mixture was stirred under H₂ atmosphere at balloon pressure at room temperature for 3 h. The reaction was then filtered through sintered funnel and the filtrate was concentrated.

### Method AA

The crude was purified by Reverse Phase Prep-HPLC to obtain compound Example 203 as a white solid (25 mg, 99.42% purity, 20% yield). RP method: Preparative HPLC was done in Waters BGM 2545 equipped with PDA detector set to multiple-wavelength UV (200-400 nm) detection. Column name: YMC Actus Triart C18 (250×20 mm, 5μ) operating at ambient temperature and flow rate of 16 mL/min. Mobile phase: A=20 mM Ammonium Bicarbonate in water, Mobile phase B=Acetonitrile. Gradient profile: Mobile phase initial composition of 60% A and 40% B, then to 20% A and 80% B in 18 min, then to 0% A and 100% B in 19 min, held in this composition up to 22 min for column washing, then returned to initial composition in 22.5 min and held for 25 min. LCMS: m/z found 400.21 [M+H]⁺, rt=2.74 min (Method 2) [Waters Acquity BEH C8 column (1.7 μm, 50×2.1 mm)]; ¹H NMR (400 MHz, DMSO-d₆): δ 9.32 (d, J=0.9 Hz, 1H), 8.12 (s, 1H), 7.75 (s, 1H), 7.69 (d, J=9.6 Hz, 1H), 7.48 (dd, J=2.0, 9.5 Hz, 1H), 7.31 (d, J=8.6 Hz, 2H), 6.87 (d, J=8.7 Hz, 2H), 5.89 (q, J=8.8 Hz, 1H), 3.71 (s, 3H), 2.75 (s, 3H).

### Example 204

### (R)—N-(1-(4-chlorophenyl)-2,2,2-trifluoroethyl)-N-methyl-[1,2,4]triazolo[4,3-c]pyrimidine-7-sulfonamide

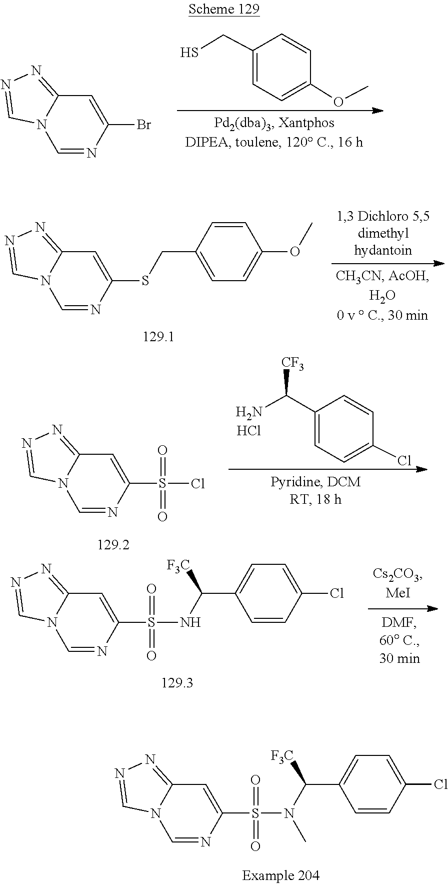

Synthesis of 7-((4-methoxybenzyl)thio)-[1,2,4]triazolo[4,3-c]pyrimidine 129.1: Intermediate 129.1 was synthesized from 7-bromo-[1,2,4]triazolo[4,3-c]pyrimidine following a method analogous to that described in Method AA using (4-methoxyphenyl)methanethiol. The crude was purified by column chromatography over silica gel using 20% ethyl acetate in hexane to yield compound 129.1 as a yellow gum (400 mg, 90% yield). LCMS: m/z found 273.2 [M+H]⁺, rt=2.05 min (Method 12) [YMC Triart C18 column (3 μm, 33×2.1 mm)].

Synthesis of [1,2,4]triazolo[4,3-c]pyrimidine-7-sulfonyl chloride 129.2: Sulfonyl chloride 129.2 was synthesized from intermediate 129.1 following the procedure described in Method B. The crude sulfonyl chloride was used immediately in the forwarding step without further purification.

Synthesis of (R)—N-(1-(4-chlorophenyl)-2,2,2-trifluoroethyl)-[1,2,4]triazolo[4,3-c]pyrimidine-7-sulfonamide 129.3: Sulfonamide 129.3 was synthesized from sulfonyl chloride 129.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 129.3 as a yellow gum (110 mg, 30% yield). LCMS: m/z found 392.0 [M+H]⁺, rt=1.79 min (Method 3) [YMC Triart C18 column (3 μm, 33×2.1 mm)].

(R)—N-(1-(4-chlorophenyl)-2,2,2-trifluoroethyl)-N-methyl-[1,2,4]triazolo[4,3-c]pyrimidine-7-sulfonamide: The methylation of intermediate 129.3 was preformed following the protocol as described in Method AD. The crude was purified by column chromatography over silica gel using 25% ethyl acetate-hexane to obtain compound Example 204 as an off-white solid (40 mg, 96.32% purity, 35% yield). LCMS: m/z found 406.2 [M+H]⁺, rt=3.20 min (Method 2) [Waters Acquity BEH C8 column (1.7 μm, 50×2.1 mm)]; ¹H NMR (400 MHz, DMSO-d₆): δ 10.00 (s, 1H), 8.92 (s, 1H), 8.50 (s, 1H), 7.48 (q, J=8.7 Hz, 4H), 6.01 (q, J=8.5 Hz, 1H), 2.84 (s, 3H).

### Example 205

### (R)—N-(1-(4-chlorophenyl)-2,2,2-trifluoroethyl)-N-methyl-[1,2,4]triazolo[4,3-b]pyridazine-6-sulfonamide

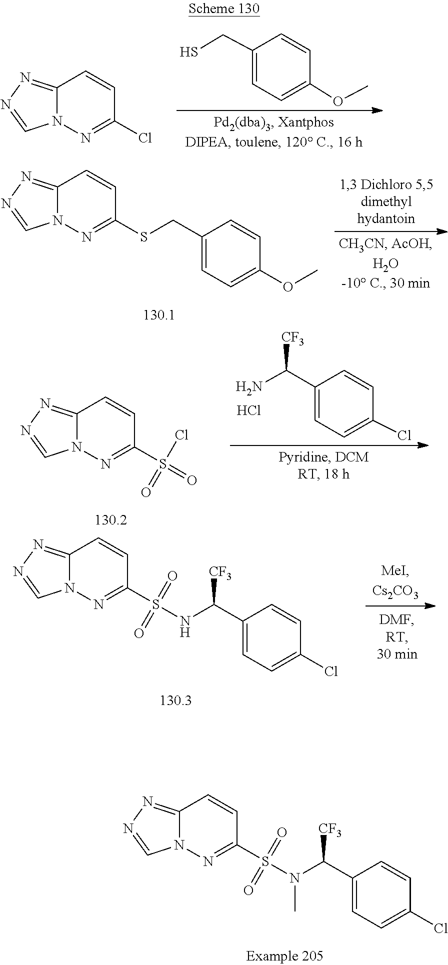

Synthesis of 6-((4-methoxybenzyl)thio)-[1,2,4]triazolo[4,3-b]pyridazine 130.1: Intermediate 130.1 was synthesized from 6-chloro-[1,2,4]triazolo[4,3-b]pyridazine following a method analogous to that described in Method AA using (4-methoxyphenyl)methanethiol. The crude was purified by column chromatography over silica gel using 20% ethyl acetate in hexane to yield compound 130.1 as a yellow sticky solid (3.0 g, 68% yield). LCMS: m/z found 273.3 [M+H]⁺, rt=1.89 min (Method 25) [Waters Acquity BEH C18 column (1.7 μm, 50×2.1 mm)].

Synthesis of [1,2,4]triazolo[4,3-b]pyridazine-6-sulfonyl chloride 130.2: Sulfonyl chloride 130.2 was synthesized from intermediate 130.1 following the procedure described in Method AB. The crude sulfonyl chloride was used immediately in the forwarding step without further purification.

Synthesis of (R)—N-(1-(4-chlorophenyl)-2,2,2-trifluoroethyl)-[1,2,4]triazolo[4,3-b]pyridazine-6-sulfonamide 130.3: Sulfonamide 130.3 was synthesized from sulfonyl chloride 130.2 following the protocol as described in Method AC using (R)-1-(4-chlorophenyl)-2,2,2-trifluoroethan-1-amine hydrochloride. The compound was purified by column chromatography over silica gel using 40% ethyl acetate in hexane to isolate desired compound 130.3 as a yellow gum (95 mg, 5% yield). LCMS: m/z found 392.0 [M+H]⁺, rt=1.74 min (Method 3) [YMC Triart C18 column (3 μm, 33×2.1 mm)].

Synthesis of (R)—N-(1-(4-chlorophenyl)-2,2,2-trifluoroethyl)-N-methyl-[1,2,4]triazolo[4,3-b]pyridazine-6-sulfonamide: The methylation of intermediate 130.3 was preformed following the protocol as described in Method AD at RT. The crude residue was purified by column chromatography over silica gel using 30% ethyl acetate in hexane to obtain compound Example 205 as a light brown sticky solid (15 mg, 96.69% purity, 15% yield). LCMS: m/z found 406.18 [M+H]⁺, rt=3.13 min (Method 3) [YMC Triart C18 column (3 μm, 33×2.1 mm)]; ¹H NMR (400 MHz, DMSO-d₆): δ 9.81 (s, 1H), 8.67 (d, J=9.8 Hz, 1H), 7.81 (d, J=9.7 Hz, 1H), 7.51 (s, 4H), 6.15-5.99 (m, 1H), 2.90 (s, 3H).

### Example 206

### (R)—N-(1-(4-chlorophenyl)-2,2-difluoroethyl)-5-cyano-N-methylpyridine-3-sulfonamide

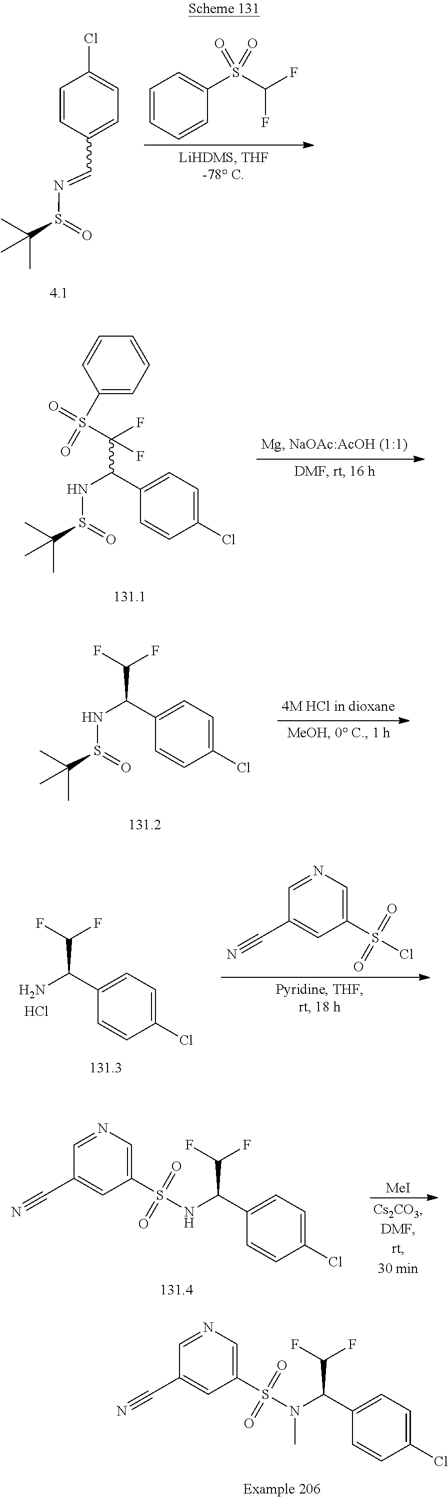

### Synthesis of (S)—N-(1-(4-chlorophenyl)-2,2-difluoro-2-(phenylsulfonyl)ethyl)-2-methylpropane-2-sulfinamide 131.1

A solution of intermediate 4.1 (1.0 g, 4.10 mmol) and ((difluoromethyl)sulfonyl)benzene (0.79 g, 4.10 mmol) in THF (15 mL) was cooled at −78° C. 1 M solution of LiHMDS in THF (4.3 mL, 4.31 mmol) was added at −78° C. and the reaction mixture was stirred at same temperature for 1 h. The reaction was then quenched with saturated solution of NH₄Cl (20 mL) was added and it was extracted with EtOAc (3×50 mL). Combined organic layer was washed with saturated solution of NH₄Cl (25 mL) and brine (25 mL), dried over anhydrous Na₂SO₄ and the solvent was evaporated under reduced pressure to obtain crude compound was purified by combi-flash column chromatography using 40% EtOAc in hexane to obtain desired product 131.1 as an off-white solid (1.2 g, 67% yield). LCMS: m/z found (436.05 [M+H]⁺) rt=1.84 min (Method 3) [YMC Triart C18 column (3 μm, 33×2.1 mm).

Synthesis of (S)—N—((R)-1-(4-chlorophenyl)-2,2-difluoroethyl)-2-methylpropane-2-sulfinamide 131.2: To a stirred solution of Intermediate 131.1 (1.2 g, 2.75 mmol) in DMF (15 mL) was added buffer solution of CH₃COONa (8.5 g) and CH₃COOH (8.5 mL) (1:1) in water (16 mL) at 0° C. Mg-turnings (1 g, 41.29 mmol) was added at portionwise, and the reaction mixture was stirred at room temperature for 16 h. The reaction mixture was quenched with cold water (30 mL) and it was extracted with EtOAc (2×50 mL). The combined organic layer was washed with water (30 mL) and brine (30 mL); dried over anhydrous Na₂SO₄ and the solvent was evaporated under reduced pressure. The crude mass was purified by prep-HPLC to obtain desired product as off-white solid 131.2 (220 mg, 27% yield). LCMS: m/z found 296.0 [M+H]⁺, rt=1.63 min (Method 3) [YMC Triart C18 column (3 μm, 33×2.1 mm)].

Synthesis of (R)-1-(4-chlorophenyl)-2,2-difluoroethan-1-amine hydrochloride salt 131.3: To a stirred solution of compound 131.2 (0.2 g, 0.68 mmol) in MeOH (3 mL) was added 4M HCl in dioxane (2 mL) at 0° C. and was stirred for 1 h. The volatiles were evaporated under reduced pressure and crude mass was triturated with diethyl ether to obtain desired product as off-white solid (150 mg). LCMS: m/z found 191.8 [M+H]⁺, rt=1.08 min (Method 3) [YMC Triart C18 column (3 μm, 33×2.1 mm)].

Synthesis of (R)—N-(1-(4-chlorophenyl)-2,2-difluoroethyl)-5-cyanopyridine-3-sulfonamide 131.4: Intermediate 131.4 was synthesized from 5-cyanopyridine-3-sulfonyl chloride following the protocol as described in Method A using (R)-1-(4-chlorophenyl)-2,2-difluoroethan-1-amine hydrochloride salt 131.3. The compound was purified by column chromatography over silica gel using 35% ethyl acetate in hexane to yield the desired compound as off-white solid (110 mg, 46% yield). LCMS: m/z found 358.0 [M+H]⁺, rt=1.64 min (Method 3) [YMC Triart C18 column (3 μm, 33×2.1 mm].

Synthesis of (R)—N-(1-(4-chlorophenyl)-2,2-difluoroethyl)-5-cyano-N-methylpyridine-3-sulfonamide: The methylation of intermediate 131.4 was preformed following the protocol as described in Method D. The crude was purified by column chromatography over silica gel using 1:5 ethyl acetate-hexane and the desired product Example 206 as a colorless oil (90 mg, 78% yield, 99.93% purity). LCMS: m/z found 372.18 [M+H]⁺, rt=2.97 min (Method 2) [Waters Acquity BEH C8 column (1.7 μm, 50×2.1 mm)]. ¹H NMR (400 MHz, Chloroform-d): δ 9.20 (d, J=1.9 Hz, 1H), 9.04 (d, J=2.0 Hz, 1H), 8.33 (s, 1H), 7.41 (d, J=8.4 Hz, 2H), 7.32 (d, J=8.4 Hz, 2H), 6.44-5.93 (m, 1H), 5.51-5.38 (m, 1H), 2.80 (s, 3H).

### Examples 207 to 209

### (R)—N-(1-(4-chlorophenyl)-2,2,2-trifluoroethyl)-6-methoxy-N-methylpyridazine-3-sulfonamide (Example 207), (R)—N-(1-(4-chlorophenyl)-2,2,2-trifluoroethyl)-6-hydroxy-N-methylpyridazine-3-sulfonamide (Example 208) and (R)—N-(1-(4-chlorophenyl)-2,2,2-trifluoroethyl)-N-methylpyridazine-3-sulfonamide (Example 209)

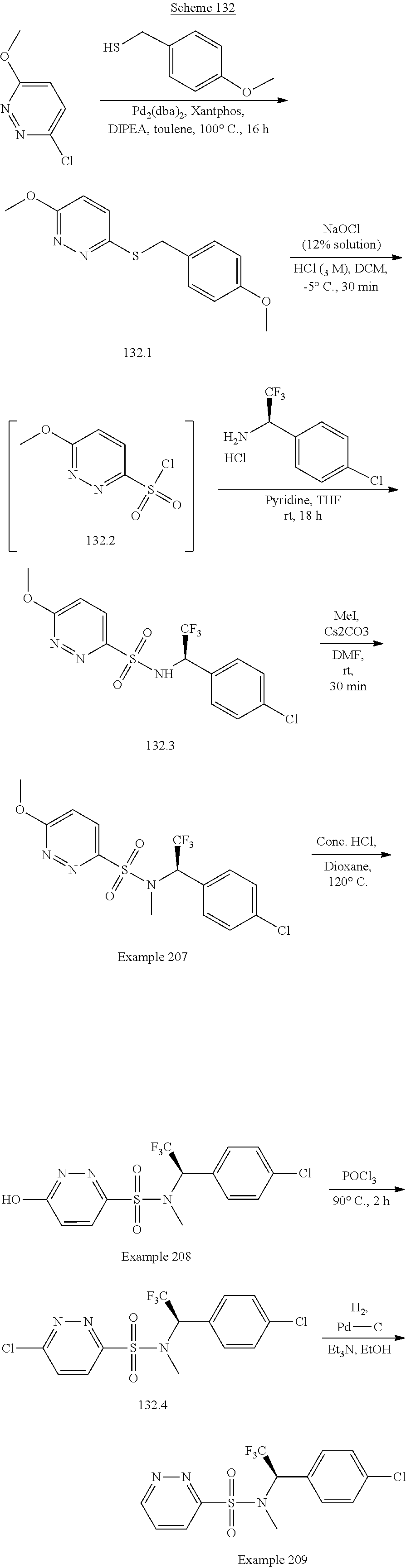

Synthesis of 3-methoxy-6-((4-methoxybenzyl)thio)pyridazine 132.1: Intermediate 132.1 was synthesized from 3-chloro-6-methoxypyridazine following a method analogous to that described in Method A using (4-methoxyphenyl)methanethiol. The crude compound was triturated with pentane and the compound 132.1 (4.0 g, 64% yield) was used in the forwarding step without further purification. LCMS: m/z found 399.9 [M+H]⁺, rt=1.89 min (Method 3) [YMC Triart C18 column (3 μm, 33×2.1 mm)].

Synthesis of 6-methoxypyridazine-3-sulfonyl chloride 2.2 Method B-I: Intermediate 132.1 (100 mg, 0.29 mmol) was dissolved in DCM (1 mL) and cooled in ice-salt bath. The solution was degasified with argon and 3N HCl (0.5 mL) was added to it and further degasified. NaOCl (12% solution in water) (0.5 mL) was added to the solution dropwise with vigorous stirring. The reaction mixture was then stirred at that temperature under inert atmosphere for 30 mins. It was then diluted with cold water (5 mL) and extracted with DCM (5 mL). The organic layer was separated, dried over anhydrous Na₂SO₄, and filtered. The filtrate was used in the forwarding step without evaporation.

Synthesis of (R)—N-(1-(4-chlorophenyl)-2,2,2-trifluoroethyl)-6-methoxypyridazine-3-sulfonamide 132.3: Sulfonamide 132.3 was synthesized from sulfonyl chloride 132.2 following the protocol as described in Method A using (R)-1-(4-chlorophenyl)-2,2,2-trifluoroethan-1-amine hydrochloride. The compound was purified by column chromatography over silica gel using 40% ethyl acetate in hexane to isolate desired compound 132.3 as yellow sticky solid (150 mg, 11% yield). LCMS: m/z found 382.0 [M+H]⁺, rt=1.69 min (Method 3) [YMC Triart C18 column (3 μm, 33×2.1 mm)].

Synthesis of (R)—N-(1-(4-chlorophenyl)-2,2,2-trifluoroethyl)-6-methoxy-N-methylpyridazine-3-sulfonamide: The methylation of intermediate 132.3 was performed following the protocol as described in Method D at room temperature. The crude residue was purified by column chromatography over silica gel using 5:1 ethyl acetate-hexane to obtain the title compound as a colorless gum (70 mg, 64% yield, 99.27% purity). LCMS: m/z found 396.20 [M+H]⁺, rt=3.00 min (Method 2) [Waters Acquity BEH C8 column (1.7 μm, 50×2.1 mm)]. ¹H NMR (400 MHz, DMSO-d₆) δ 8.15 (d, J=9.2 Hz, 1H), 7.54-7.44 (m, 3H), 7.37 (d, J=8.4 Hz, 2H), 6.01 (q, J=8.5 Hz, 1H), 4.12 (s, 3H), 2.89 (s, 3H).

Synthesis of (R)—N-(1-(4-chlorophenyl)-2,2,2-trifluoroethyl)-6-hydroxy-N-methylpyridazine-3-sulfonamide: Compound Example 207 (50 mg) was dissolved in 1,4-dioxane (0.5 mL) and to the solution was added conc. HCl. (0.1 mL). The reaction mixture was heated at 110° C. for 1 h. It was then diluted with water (2 mL) and extracted with 10% MeOH in DCM (2×5 mL). The organic part was washed with water, dried (anhydrous Na₂SO₄) and the solvent was evaporated under reduced pressure. The crude product was purified by Reverse Phase Prep-HPLC. Reverse Phase Prep-HPLC method: Preparative HPLC Was done in WATERS BGM 2545 EQUIPPED with WATERS PDA detector 2998 set to multiple-wavelength UV (200-400 nm) detection. Column name: YMC TRIART C18 (250×20 mm, 5μ) operating at ambient temperature and flow rate of 16 mL/min. Mobile phase: A=20 mM ammonium bicarbonate in water, B=Acetonitrile. Gradient profile: Mobile phase initial composition of 80% A and 20% B, then to 60% A and 40% B in 3 min, then to 20% A and 80% B in 22 min, then to 0% A and 100% B in 22.5 min, held in this composition up to 25 min for column washing, then returned to initial composition in 25.5 min and held till 28 min. LCMS: m/z found 382.17 [M+H]⁺, rt=2.83 min (Method 2) [Waters Acquity BEH C8 column (1.7 μm, 50×2.1 mm)]. ¹H NMR (400 MHz, DMSO-d₆) δ 13.88-13.61 (m, 1H), 7.83 (d, J=9.9 Hz, 1H), 7.52 (d, J=8.6 Hz, 2H), 7.46 (d, J=8.6 Hz, 2H), 7.07 (d, J=10.0 Hz, 1H), 5.98 (q, J=8.0 Hz, 1H), 2.80 (s, 3H).

Synthesis of (R)-6-chloro-N-(1-(4-chlorophenyl)-2,2,2-trifluoroethyl)-N-methylpyridazine-3-sulfonamide 132.4: Compound Example 208 (130 mg, crude) was heated with POCl₃ (2 mL) at 90° C. for 2 h. Excess POCl₃ was evaporated under reduced pressure. The crude was cooled in ice bath and ice water (5 mL) was added slowly. Aq. saturated NaHCO₃ solution (10 mL) was then added to it slowly until effervescence ceased. It was then extracted with DCM (2×10 mL). The organic part was washed with water (10 mL) and NaHCO₃ (10 mL) solution, dried (anhydrous Na₂SO₄) and was concentrated under reduced pressure. The compound was further purified by column chromatography over silica gel using 10% ethyl acetate in hexane to yield the compound 132.4 (100 mg, 73% yield) as a yellow gum. LCMS: m/z found 263.0 [M+H]⁺, rt=1.70 min (Method 3) [YMC Triart C18 column (3 μm, 33×2.1 mm)].

(R)—N-(1-(4-chlorophenyl)-2,2,2-trifluoroethyl)-N-methylpyridazine-3-sulfonamide: Compound 132.4 (100 mg, 0.25 mmol) was dissolved in ethanol (1.5 mL) and the solution was degasified with argon. Et₃N (1 mL) was added to it followed by Pd—C (wet, 10% w/w) (20 mg). The reaction mixture was stirred at room temperature under hydrogen atmosphere (balloon pressure) overnight. It was then filtered and concentrated under reduced pressure. The crude product was further purified by column chromatography over silica gel using 10% ethyl acetate in hexane to yield the compound Example 209 (50 mg, 99.50% purity, 55% yield) as a light-yellow gum. LCMS: m/z found 366.16 [M+H]⁺, rt=2.91 min (Method 2) [Waters Acquity BEH C8 column (1.7 μm, 50×2.1 mm)]. ¹H NMR (400 MHz, DMSO-d₆) δ 9.49 (dd, J=1.6, 5.2 Hz, 1H), 8.29 (dd, J=1.6, 8.6 Hz, 1H), 8.05 (dd, J=5.0, 8.4 Hz, 1H), 7.46 (d, J=8.6 Hz, 2H), 7.37 (d, J=8.6 Hz, 2H), 6.04 (q, J=8.5 Hz, 1H), 2.92 (s, 3H).

### Examples 210-211-212

### (R)—N-(1-(4-chlorophenyl)-2,2,2-trifluoroethyl)-N-methylimidazo[1,2-b]pyridazine-3-sulfonamide (Example 210), (R)—N-methyl-N-(2,2,2-trifluoro-1-(4-(trifluoromethyl)phenyl)ethyl)imidazo[1,2-b]pyridazine-3-sulfonamide (Example 211) and (R)—N-ethyl-N-(2,2,2-trifluoro-1-(4-(trifluoromethyl)phenyl)ethyl)imidazo[1,2-b]pyridazine-3-sulfonamide (Example 212)

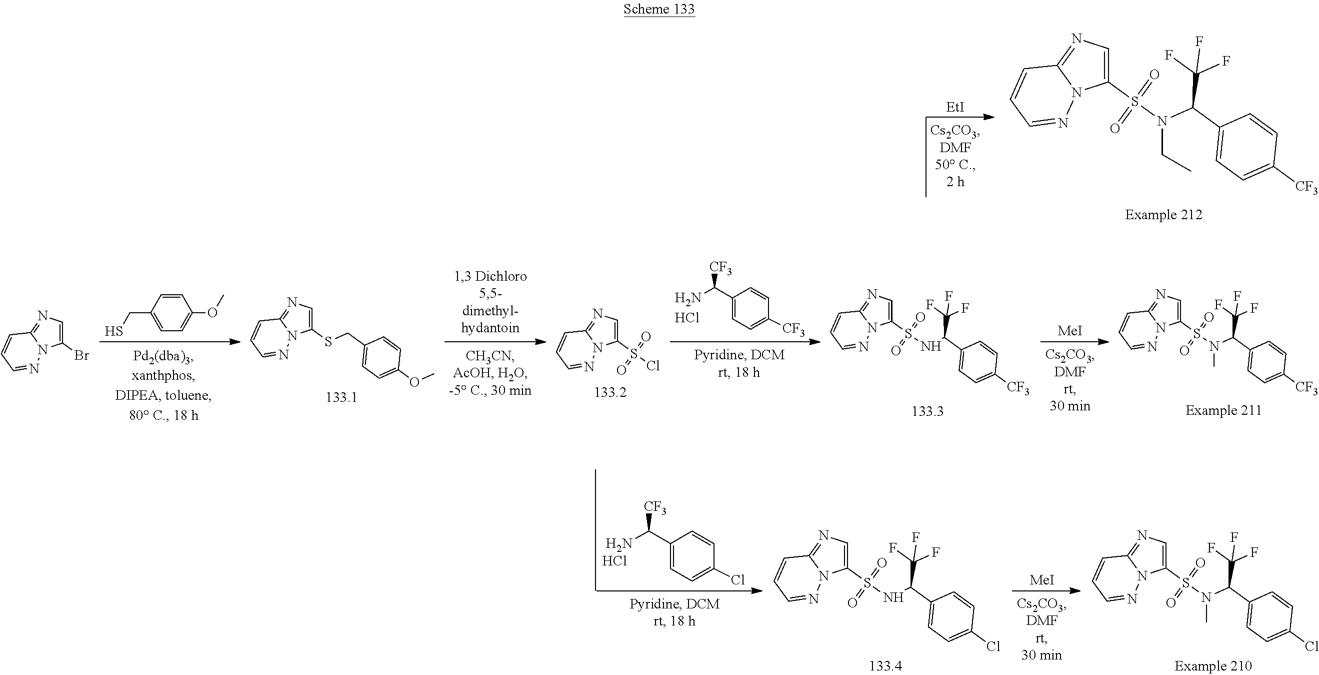

Synthesis of 3-((4-methoxybenzyl)thio)imidazo[1,2-b]pyridazine 133.1: Intermediate 133.1 was synthesized from 3-bromoimidazo[1,2-b]pyridazine following a method analogous to that described in Method AA using (4-methoxyphenyl)methanethiol. The crude residue was purified by column chromatography over silica gel using 40% ethyl acetate-hexane to yield compound 133.1 as yellow gum (4.0 g, 58% yield). LCMS: m/z found 272.0 [M+H]⁺, rt=1.51 min (Method 2) [YMC Triart C18 column (3 μm, 33×2.1 mm)].

Synthesis of imidazo[1,2-b]pyridazine-3-sulfonyl chloride 133.2: Sulfonyl chloride 133.2 was synthesized from intermediate 133.1 following the procedure described in Method AB. The crude sulfonyl chloride was used immediately in the forwarding step without further purification.

Synthesis of (R)—N-(2,2,2-trifluoro-1-(4-(trifluoromethyl)phenyl)ethyl)imidazo[1,2-b]pyridazine-3-sulfonamide 133.3: Sulfonamide 133.3 was synthesized from sulfonyl chloride 133.2 following the protocol as described in Method AC using (R)-2,2,2-trifluoro-1-(4-(trifluoromethyl)phenyl)ethan-1-amine hydrochloride. The compound was purified by column chromatography over silica gel using 40% ethyl acetate in hexane to isolate desired compound 133.3 as yellow gum (230 mg, 29% yield). LCMS: m/z found 425.0 [M+H]⁺, rt=1.65 min (Method 3) [YMC Triart C18 column (3 μm, 33×2.1 mm)].

Synthesis of (R)—N-methyl-N-(2,2,2-trifluoro-1-(4-(trifluoromethyl)phenyl)ethyl)imidazo[1,2-b]pyridazine-3-sulfonamide: The methylation of intermediate 133.3 was preformed following the protocol as described in Method AD. The crude was purified by column chromatography over silica gel using 5:1 ethyl acetate-hexane to obtain compound Example 211 as off-white solid (60 mg, 25% yield). LCMS: m/z found 439.25 [M+H]⁺, rt=3.00 min (Method 2) [Waters Acquity BEH C8 column (1.7 μm, 50×2.1 mm)].

Synthesis of (R)—N-ethyl-N-(2,2,2-trifluoro-1-(4-(trifluoromethyl)phenyl)ethyl)imidazo[1,2-b]pyridazine-3-sulfonamide: The ethylation of intermediate 133.3 was preformed following the protocol as described in Method AD. The crude was purified by column chromatography over silica gel using 5:1 ethyl acetate-hexane to obtain compound Example 212 as an off-white solid (60 mg, 25% yield). LCMS: m/z found 453.28 [M+H]⁺, rt=3.15 min (Method 2) [Waters Acquity BEH C8 column (1.7 μm, 50×2.1 mm)]. ¹H NMR (400 MHz, DMSO-d₆) δ 8.68 (d, J=3.2 Hz, 1H), 8.37-8.28 (m, 2H), 7.61 (d, J=8.2 Hz, 2H), 7.54-7.44 (m, 3H), 6.02 (q, J=8.0 Hz, 1H), 3.84-3.72 (m, 1H), 3.64-3.54 (m, 1H), 0.99 (t, J=7.0 Hz, 3H).

Synthesis of (R)—N-(2,2,2-trifluoro-1-(4-(trifluoromethyl)phenyl)ethyl)imidazo[1,2-b]pyridazine-3-sulfonamide 133.4: Sulfonamide 133.4 was synthesized from sulfonyl chloride 133.2 following the protocol as described in Method AC using (R)-(R)-1-(4-chlorophenyl)-2,2,2-trifluoroethan-1-amine hydrochloride. The compound was purified by column chromatography over silica gel using 40% ethyl acetate in hexane to isolate desired compound 133.4 as yellow gum (230 mg, 25% yield). LCMS: m/z found 391.0 [M+H]⁺, rt=1.60 min (Method 3) [YMC Triart C18 column (3 μm, 33×2.1 mm)].

Synthesis of (R)—N-(1-(4-chlorophenyl)-2,2,2-trifluoroethyl)-N-methylimidazo[1,2-b]pyridazine-3-sulfonamide: The methylation of intermediate 133.4 was preformed following the protocol as described in Method AD. The crude was purified by column chromatography over silica gel using 5:1 ethyl acetate-hexane to obtain compound Example 210 as an off-white solid (70 mg, 25% yield). LCMS: m/z found 405.20 [M+H]⁺, rt=2.92 min (Method 2) [Waters Acquity BEH C8 column (1.7 μm, 50×2.1 mm)]. ¹H NMR (400 MHz, DMSO) δ 8.69 (dd, J=4.4, 1.2 Hz, 1H), 8.38-8.32 (m, 2H), 7.51 (dd, J=9.3, 4.4 Hz, 1H), 7.36 (d, J=8.5 Hz, 2H), 7.27 (d, J=8.5 Hz, 2H), 5.94 (q, J=8.5 Hz, 1H), 3.00 (s, 3H).

### Example 213

### (R)—N-(1-(4-chlorophenyl)-2-fluoroethyl)-5-cyano-N-methylpyridine-3-sulfonamide

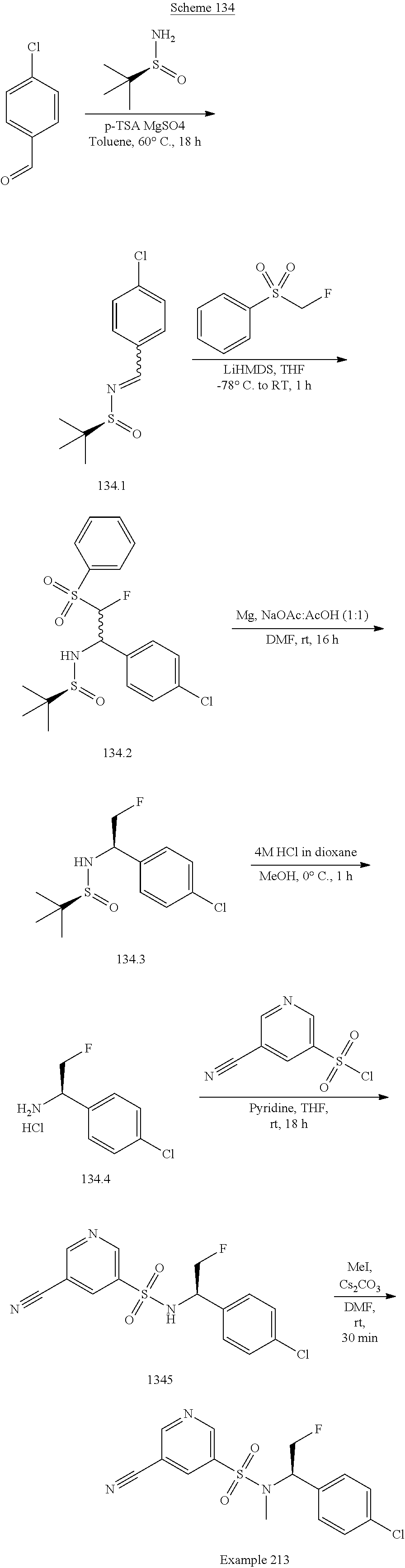

Synthesis of (S)—N-(4-chlorobenzylidene)-2-methylpropane-2-sulfinamide 134.1: To a stirred solution of 4-chlorobenzaldehyde (5.0 g, 35.57 mmol) and (S)-2-methylpropane-2-sulfinamide (5.17 g, 42.68 mmol) in toluene (30 mL) p-TSA was added (0.34 g, 1.78 mmol) followed by anhydrous MgSO₄. The reaction mixture was stirred at 60° C. for 16 h. After the completion of reaction (confirmed by TLC analysis) water (40 mL) was added to it and was extracted with EtOAc (3×50 mL). Combined organic layer was washed with water (20 mL) and brine (20 mL), dried over anhydrous Na₂SO₄ and the solvent was removed under reduced pressure to obtain crude compound 134.1 (8.0 g, 92% yield). This crude mass was directly used in forwarding step without further purification. LCMS: m/z found (244.1 [M+H]⁺, rt=2.12 min (Method 11) [Waters Acquity BEH C18 column (1.7 μm, 50×2.1 mm).

Synthesis of (S)—N-(1-(4-chlorophenyl)-2-fluoro-2-(phenylsulfonyl)ethyl)-2-methylpropane-2-sulfinamide 134.2: A solution of intermediate 134.1 (1.0 g, 4.4 mmol) and ((fluoromethyl)sulfonyl)benzene (0.76 g, 4.4 mmol) in THF (15 mL) was cooled at −78° C. 1 M solution of LiHMDS in THF (4.3 mL, 4.51 mmol) was added at −78° C. and the reaction mixture was stirred at same temperature for 1 h. The reaction was then quenched with saturated solution of NH₄Cl (20 mL) was added and it was extracted with EtOAc (3×50 mL). Combined organic layer was washed with saturated solution of NH₄Cl (25 mL) and brine (25 mL), dried over anhydrous Na₂SO₄ and the solvent was evaporated under reduced pressure to obtain crude compound 134.2 which was used in the forwarding step without any purification.

Synthesis of (S)—N—((R)-1-(4-chlorophenyl)-2-fluoroethyl)-2-methylpropane-2-sulfinamide 134.3. To a stirred solution of Intermediate 134.2 (1.2 g, 2.8 mmol) in DMF (15 mL) was added buffer solution of CH₃COONa (8.5 g) and CH₃COOH (8.5 mL) (1:1) in water (16 mL) at 0° C. Mg-turnings (0.96 g, 40.28 mmol) was added at portion-wise, and the reaction mixture was stirred at room temperature for 16 h. The reaction mixture was quenched with cold water (30 mL) and it was extracted with EtOAc (2×50 mL). The combined organic layer was washed with water (30 mL) and brine (30 mL), dried over anhydrous Na₂SO₄ and the solvent was evaporated under reduced pressure. The crude product was purified by column chromatography over silica gel using 20% ethyl acetate in hexane to obtain desired product 134.3 as an off-white solid (250 mg, 31% yield). LCMS: m/z found 278.0 [M+H]⁺, rt=1.61 min (Method 3) [YMC Triart C18 column (3 μm, 33×2.1 mm)].

Synthesis of (R)-1-(4-chlorophenyl)-2-fluoroethan-1-amine hydrochloride 134.4: To a stirred solution of intermediate 134.3 (0.25 g, 0.903 mmol) in MeOH (3 mL) 4M HCl in dioxane (2 mL) at 0° C. was added and the reaction mixture was stirred for 1 h. The volatiles were evaporated under reduced pressure and crude mass was triturated with diethyl ether (20 mL) to obtain desired product 134.4 as an off-white solid (150 mg). ¹H NMR (400 MHz, DMSO-d₆): δ 8.97 (s, 2H), 7.58 (q, J=8.5 Hz, 4H), 4.85-4.73 (m, 1H), 4.70 (s, 2H).

Synthesis of (R)—N-(1-(4-chlorophenyl)-2-fluoroethyl)-5-cyanopyridine-3-sulfonamide 134.5: Intermediate 134.5 was synthesized from 5-cyanopyridine-3-sulfonyl chloride following the protocol as described in Method A using (R)-1-(4-chlorophenyl)-2-fluoroethan-1-amine hydrochloride. The compound was purified by column chromatography over silica gel using 35% ethyl acetate in hexane to yield the desired compound 134.5 as a white solid (33 mg, 39% yield). LCMS: m/z found 340.0 [M+H]⁺, rt=0.905 min (Method 3) YMC Triart C18 column (3 μm, 33×2.1 mm).

Synthesis of (R)—N-(1-(4-chlorophenyl)-2-fluoroethyl)-5-cyano-N-methylpyridine-3-sulfonamide: The methylation of intermediate 134.5 was preformed following the protocol as described in Method D. The crude was purified by column chromatography over silica gel using 1:5 ethyl acetate-hexane and the compound Example 213 was obtained as a white solid (15 mg, 98.96% purity, 43% yield). LCMS: m/z found 354.15 [M+H]⁺, rt=3.39 min (Method 2) [Waters Acquity BEH C8 column (1.7 μm, 50×2.1 mm)]; ¹H NMR (400 MHz, DMSO-d₆): δ 9.28 (d, J=1.8 Hz, 1H), 9.20 (d, J=2.0 Hz, 1H), 8.77 (t, J=2.0 Hz, 1H), 7.46 (d, J=8.4 Hz, 2H), 7.34 (d, J=8.5 Hz, 2H), 5.51-5.39 (m, 1H), 4.86-4.76 (m, 1H), 4.73-4.65 (m, 1H), 2.84 (s, 3H).

### Example 214

### (R)—N-methyl-N-(2,2,2-trifluoro-1-(4-(trifluoromethyl)phenyl)ethyl)imidazo[1,2-a]pyrimidine-3-sulfonamide

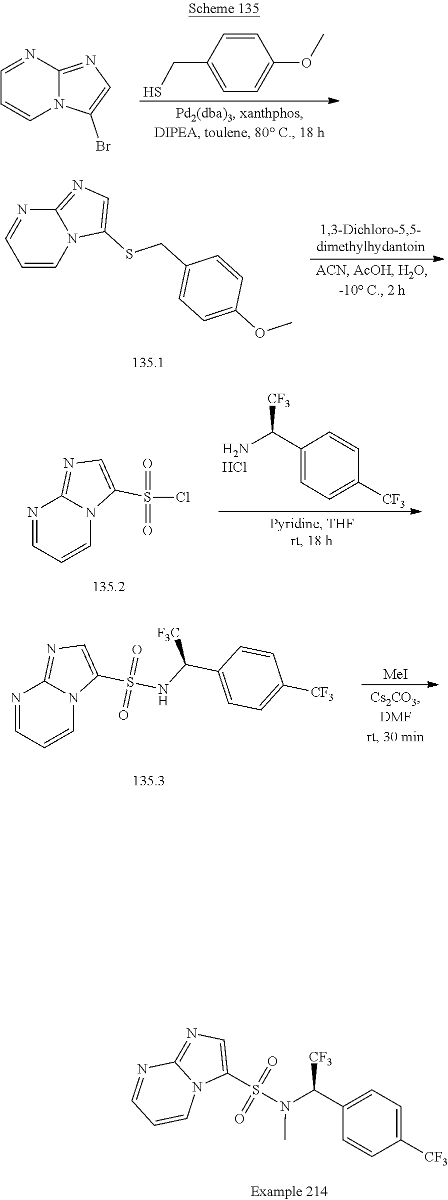

Synthesis of 3-((4-methoxybenzyl)thio)imidazo[1,2-a]pyrimidine 135.1: Intermediate 135.1 was synthesized from 3-bromoimidazo[1,2-a]pyrimidine following a method analogous to that described in Method AA using (4-methoxyphenyl)methanethiol. The reaction mixture was then filtered through a sintered funnel and the residue was collected, concentrated to a residue. The crude residue was purified by column chromatography over silica gel using 1:4 ethyl acetate-hexane to yield the intermediate 135.1 as a brown oil (2.0 g, 87% yield). LCMS m/z found 272.0 [M+H]⁺, rt=1.36 min (Method 3) [YMC Triart C18 column (3 μm, 33×2.1 mm)].

Synthesis of imidazo[1,2-a]pyrimidine-3-sulfonyl chloride 135.2: Sulfonyl chloride 135.2 was synthesized from intermediate 135.1 following the procedure described in Method AB. The compound was purified by column chromatography over silica gel using 3:7 ethyl acetate:hexane to obtain compound as white solid (2.4 g, 62% yield). LCMS m/z found 217.8 [M+H]⁺, rt=1.28 min (Method 3) [YMC Triart C18 column (3 μm, 33×2.1 mm)].

Synthesis of (R)—N-(2,2,2-trifluoro-1-(4-(trifluoromethyl)phenyl)ethyl)imidazo[1,2-a]pyrimidine-3-sulfonamide 135.3: Intermediate 135.3 was synthesized from sulfonyl chloride 135.2 following the protocol as described in Method AC using (R)-1-(4-chlorophenyl)-2,2,2-trifluoroethan-1-amine hydrochloride in THF solvent. The compound was purified by column chromatography over silica gel using 3:7 ethyl acetate:hexane to provide compound 135.3 as an off-white solid (300 mg, 30% yield). LCMS m/z found 425.20 [M+H]⁺, rt=2.01 min (Method 23) [Waters Acquity BEH C18 column (1.7 μm, 50×2.1 mm)].

Synthesis of (R)—N-methyl-N-(2,2,2-trifluoro-1-(4-(trifluoromethyl)phenyl)ethyl)imidazo[1,2-a]pyrimidine-3-sulfonamide: The methylation of intermediate 135.3 was preformed following the protocol as described in Method AD. The crude was purified by column chromatography over silica gel using 1:4 ethyl acetate-hexane to obtain compound Example 214 as a colorless oil (130 mg, 99.09% purity, 42% yield). ¹H NMR (400 MHz, DMSO-d6): δ 9.17 (dd, J=2.0, 7.2 Hz, 1H), 8.83 (dd, J=2.0, 4.4 Hz, 1H), 8.49 (s, 1H), 7.78 (d, J=8.4 Hz, 2H), 7.63 (d, J=8.0 Hz, 2H), 7.38 (dd, J=4.4, 6.8 Hz, 1H), 6.33 (q, J=8.4 Hz, 1H), 2.86 (s, 3H).

### Example 215 and Example 216

### (R)—N-methyl-N-(2,2,2-trifluoro-1-(4-(trifluoromethyl)phenyl)ethyl)-[1,2,4]triazolo[1,5-a]pyridine-7-sulfonamide (Example 215) and (R)—N-ethyl-N-(2,2,2-trifluoro-1-(4-(trifluoromethyl)phenyl)ethyl)-[1,2,4]triazolo[1,5-a]pyridine-7-sulfonamide (Example 216)

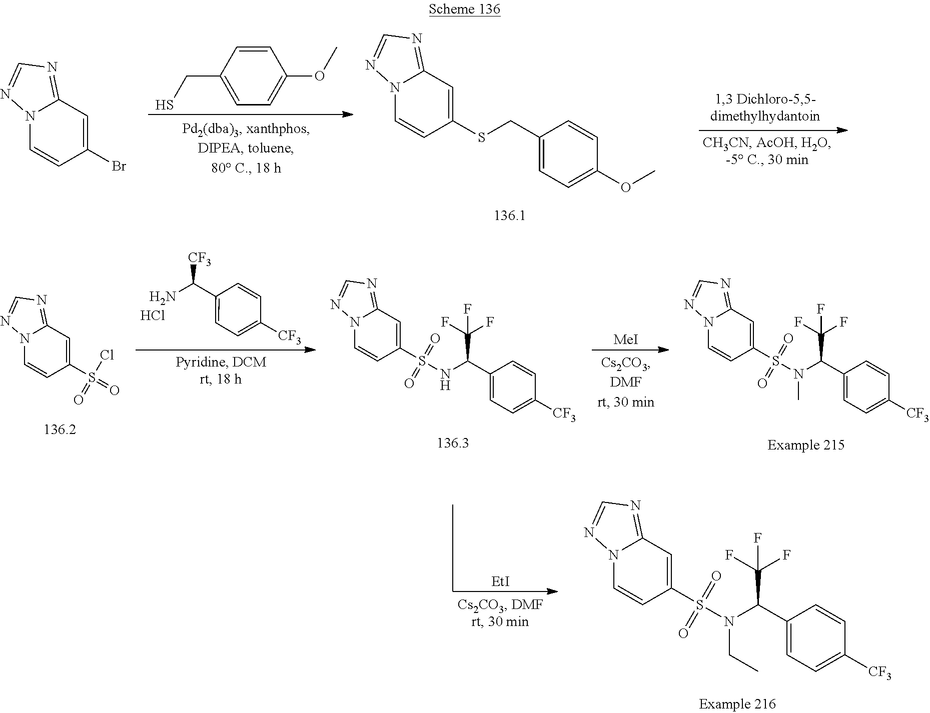

Synthesis of 7-((4-methoxybenzyl)thio)-[1,2,4]triazolo[1,5-a]pyridine 136.1: Intermediate 136.1 was synthesized from 7-bromo-[1,2,4]triazolo[1,5-a]pyridine following a method analogous to that described in Method AA using (4-methoxyphenyl)methanethiol. The reaction was then filtered through sintered funnel and the residue was collected, concentrated to a residue. The crude was purified by column chromatography over silica gel using 1:4 ethyl acetate-hexane to yield the intermediate 136.1 as a brown gum (2.42 g, 87% yield). LCMS m/z found 272.0 [M+H]⁺, rt=1.36 min (Method 3) [YMC Triart C18 column (3 μm, 33×2.1 mm)].

Synthesis of [1,2,4]triazolo[1,5-a]pyridine-7-sulfonyl chloride 136.2: Sulfonyl chloride 136.2 was synthesized from intermediate 136.1 following the procedure described in Method AB. The crude sulfonyl chloride was used immediately in the forwarding step without further purification.

Synthesis of (R)—N-(2,2,2-trifluoro-1-(4-(trifluoromethyl)phenyl)ethyl)-[1,2,4]triazolo[1,5-a]pyridine-7-sulfonamide 136.3: Intermediate 136.3 was synthesized from sulfonyl chloride 136.2 following the protocol as described in Method AC using (R)-2,2,2-trifluoro-1-(4-(trifluoromethyl)phenyl)ethan-1-amine hydrochloride. The compound was purified by column chromatography over silica gel using 3:7 ethyl acetate:hexane to provide compound 136.3 as off-white solid (300 mg, 20% yield). LCMS m/z found 272.0 [M+H]⁺, rt=1.36 min (Method 3) [YMC Triart C18 column (3 μm, 33×2.1 mm)].

Synthesis of (R)—N-methyl-N-(2,2,2-trifluoro-1-(4-(trifluoromethyl)phenyl)ethyl)-[1,2,4]triazolo[1,5-a]pyridine-7-sulfonamide: The methylation of intermediate 7.3 was preformed following the protocol as described in Method AD. The crude was purified by column chromatography over silica gel using 1:4 ethyl acetate-hexane to obtain compound Example 215 as a colorless oil (180 mg, 99.08% purity, 70% yield). ¹H NMR (400 MHz, DMSO): δ 9.22 (d, J=7.2 Hz, 1H), 8.78 (s, 1H), 8.48 (s, 1H), 7.78 (d, J=8.2 Hz, 2H), 7.69-7.58 (m, 3H), 6.33 (q, J=8.3 Hz, 1H), 2.87 (s, 3H).

Synthesis of (R)—N-ethyl-N-(2,2,2-trifluoro-1-(4-(trifluoromethyl)phenyl)ethyl)-[1,2,4]triazolo[1,5-a]pyridine-7-sulfonamide: The ethylation of intermediate 136.3 was preformed following the protocol as described in Method AD. The crude was purified by column chromatography over silica gel using 1:4 ethyl acetate-hexane to obtain compound Example 216 as a colorless oil (40 mg, 97.70% purity). ¹H NMR (400 MHz, DMSO): δ 9.19 (d, J=7.2 Hz, 1H), 8.76 (s, 1H), 8.49 (s, 1H), 7.77 (d, J=8.0 Hz, 2H), 7.70-7.60 (m, 3H), 6.37-6.28 (m, 1H), 3.60-3.45 (m, 1H), 3.45-3.25 (m, 1H), 1.00 (t, J=6.7 Hz, 3H).

### Example 217

### (R)-5-fluoro-N-methyl-N-(2,2,2-trifluoro-1-(4-(trifluoromethyl)phenyl)ethyl)pyridine-3-sulfonamide

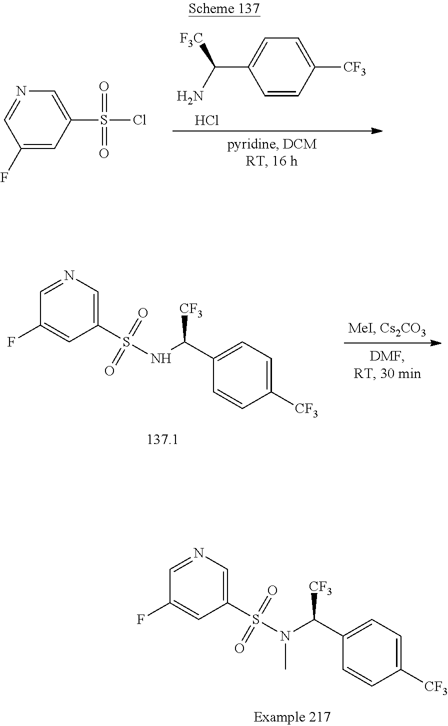 [SMILES: O=S(=O)(Cl)c1cncc(F)c1 ; N[C@H](c1ccc(C(F)(F)F)cc1)C(F)(F)F ; Cl ; O=S(=O)(N[C@H](c1ccc(C(F)(F)F)cc1)C(F)(F)F)c1cncc(F)c1 ; CN([C@H](c1ccc(C(F)(F)F)cc1)C(F)(F)F)S(=O)(=O)c1cncc(F)c1]

Synthesis of (R)-5-fluoro-N-(2,2,2-trifluoro-1-(4-(trifluoromethyl)phenyl)ethyl)pyridine-3-sulfonamide 137.1: Intermediate 137.1 was synthesized from 5-fluoropyridine-3-sulfonyl chloride following the protocol as described in Method AC using (R)-2,2,2-trifluoro-1-(4-(trifluoromethyl)phenyl)ethan-1-amine hydrochloride. The compound was purified by column chromatography over silica gel using 3:7 ethyl acetate:hexane to provide compound 137.1 as off-white solid (360 mg, 21% purity). LCMS: m/z found 403.19 [M+H]⁺, rt=3.72 min (Method 2) [Waters Acquity BEH C18 column (1.7 μm, 50×2.1 mm)].

Synthesis of (R)-5-fluoro-N-methyl-N-(2,2,2-trifluoro-1-(4-(trifluoromethyl)phenyl)ethyl)pyridine-3-sulfonamide: The methylation of intermediate 137.1 was preformed following the protocol as described in Method AD. The crude was purified by column chromatography over silica gel using 1:4 ethyl acetate-hexane to obtain compound Example 217 as a yellow gum (240 mg, 99.07% purity, 77% yield). LCMS: m/z found 417.21 [M+H]⁺, rt=3.62 min (Method 2) [Waters Acquity BEH C18 column (1.7 μm, 50×2.1 mm)]. ¹H NMR (400 MHz, DMSO) δ 8.94 (t, J=10.4 Hz, 2H), 8.34 (d, J=8.4 Hz, 1H), 7.80 (d, J=8.0 Hz, 2H), 7.61 (d, J=8.0 Hz, 2H), 6.24 (d, J=8.4 Hz, 1H), 2.86 (s, 3H).

### Example 218 to Example 224

### N-ethyl-5-fluoro-N-(2,2,2-trifluoro-1-(4-fluorophenyl)ethyl)pyridine-3-sulfonamide (Example 218), N-ethyl-5-fluoro-N-(2,2,2-trifluoro-1-(4-fluorophenyl)ethyl)pyridine-3-sulfonamide (Example 219), (R)—N-(1-(4-chlorophenyl)-2,2,2-trifluoroethyl)-5-fluoro-N-methylpyridine-3-sulfonamide (Example 220), (R)-5-fluoro-N-methyl-N-(2,2,2-trifluoro-1-(4-methoxyphenyl)ethyl)pyridine-3-sulfonamide (Example 221), (R)-5-fluoro-N-methyl-N-(2,2,2-trifluoro-1-(p-tolyl)ethyl)pyridine-3-sulfonamide (Example 222), (R)-5-fluoro-N-methyl-N-(2,2,2-trifluoro-1-(4-fluorophenyl)ethyl)pyridine-3-sulfonamide (Example 223) and (R)-5-fluoro-N-methyl-N-(2,2,2-trifluoro-1-(4-(trifluoromethyl)phenyl)ethyl)pyridine-3-sulfonamide (Example 224)

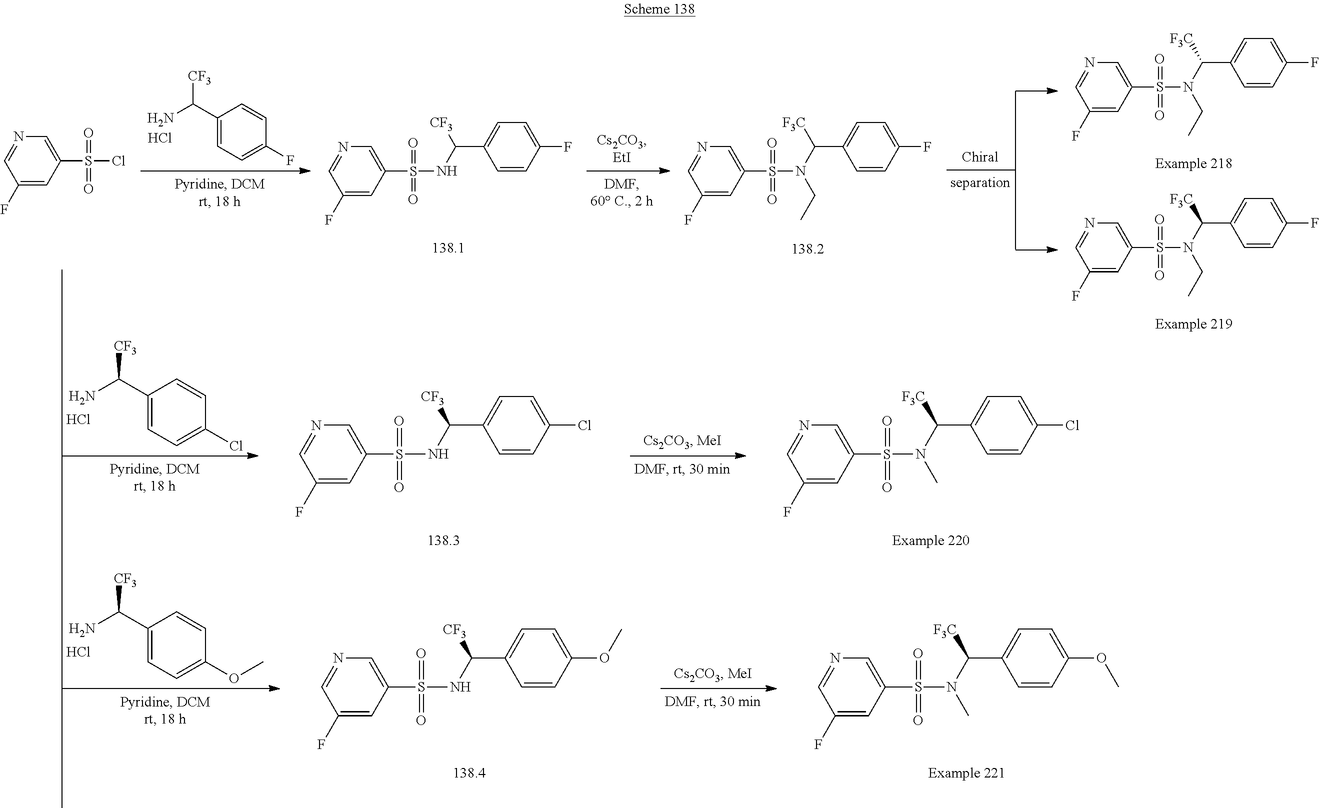

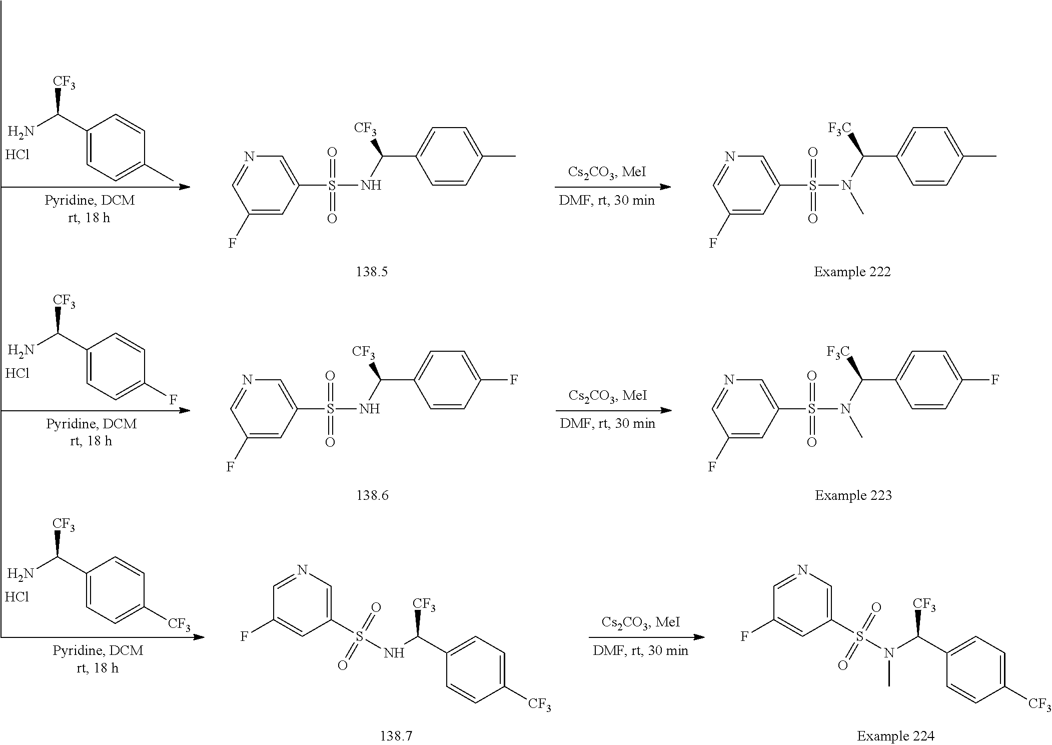

Synthesis of 5-fluoro-N-(2,2,2-trifluoro-1-(4-fluorophenyl)ethyl)pyridine-3-sulfonamide 138.1: Sulfonamide 138.1 was synthesized from 5-fluoropyridine-3-sulfonyl chloride following the protocol as described in Method O using (R)-1-(4-fluorophenyl)-2,2,2-trifluoroethan-1-amine hydrochloride. The compound was purified by column chromatography over silica gel using 20% ethyl acetate in hexane to isolate desired compound 138.1 as yellow gum (220 mg, 40% yield). LCMS: m/z found 353.0[M+H]⁺, rt=1.80 min (Method 3) [YMC Triart C18 column (3 μm, 33×2.1 mm)].

Synthesis of N-ethyl-5-fluoro-N-(2,2,2-trifluoro-1-(4-fluorophenyl)ethyl)pyridine-3-sulfonamide 138.2: The ethylation of intermediate 138.1 was performed following the protocol as described in Method D at 60° C. The crude was purified by column chromatography over silica gel using 5:1 ethyl acetate-hexane to obtain compound 138.2 as a colorless gum (175 mg, 38% yield). LCMS: m/z found 381.0 [M+H]⁺, rt=1.96 min (Method 3) [YMC Triart C18 column (3 μm, 33×2.1 mm)]. Chiral separation method: Chiral separation was done on M-4-30 instrument by using CHIRALPAK IG column (21 mm×25 cm), 5μ, operating at 35° C. temperature, maintaining flow rate of 40 ml/min, using 90% C02 in super critical state & 0.3% isopropylamine in MeOH as mobile phase, run this isocratic mixture up to 9 minutes while maintaining the isobaric condition of 100 bar at 220 nm wavelength.

[EN-1] Example 218: Yellow sticky gum (32 mg, 97.82% purity). LCMS: m/z found 381.15 [M+H]⁺, rt=3.17 min (Method 2) [Waters Acquity BEH C8 column (1.7 μm, 50×2.1 mm)]. 1H NMR (400 MHz, DMSO-d6) δ 8.94 (s, 1H), 8.89 (d, J=2.3 Hz, 1H), 8.36-8.28 (m, 1H), 7.44 (t, J=5.7, 8.2 Hz, 2H), 7.23 (t, J=8.7 Hz, 2H), 6.06 (q, J=8.4 Hz, 1H), 3.49-3.23 (m, 2H), 0.95 (t, J=6.9 Hz, 3H).

[EN-2] Example 219: Yellow sticky gum (31 mg, 94.13% purity). LCMS: m/z found 381.11 [M+H]⁺, rt=3.17 min (Method 2) [Waters Acquity BEH C8 column (1.7 μm, 50×2.1 mm)]. ¹H NMR (400 MHz, DMSO-d₆) δ 8.94 (s, 1H), 8.89 (d, J=2.4 Hz, 1H), 8.36-8.28 (m, 1H), 7.44 (dd, J=5.2, 8.7 Hz, 2H), 7.23 (t, J=8.8 Hz, 2H), 6.05 (q, J=8.6 Hz, 1H), 3.47-3.29 (m, 2H), 0.95 (t, J=6.9 Hz, 3H).

Synthesis of (R)—N-(1-(4-chlorophenyl)-2,2,2-trifluoroethyl)-5-fluoropyridine-3-sulfonamide 138.3: Sulfonamide 138.3 was synthesized from 5-fluoropyridine-3-sulfonyl chloride following the protocol as described in Method O using (R)-1-(4-chlorophenyl)-2,2,2-trifluoroethan-1-amine hydrochloride. The compound was purified by column chromatography over silica gel using 20% ethyl acetate in hexane to isolate desired compound 138.3 as a yellow gum (400 mg, 58% yield). LCMS: m/z found 368.9 [M+H]⁺, rt=1.88 min (Method 3) [YMC Triart C18 column (3 μm, 33×2.1 mm)].

Synthesis of (R)—N-(1-(4-chlorophenyl)-2,2,2-trifluoroethyl)-5-fluoro-N-methylpyridine-3-sulfonamide: The methylation of intermediate 138.3 was preformed following the protocol as described in Method D. The crude was purified by column chromatography over silica gel using 10% ethyl acetate in hexane to obtain compound Example 220 as yellow sticky gum (62 mg, 99.52% purity). LCMS: m/z found 383.21 [M+H]⁺, rt 3.18 min (Method 2) [Waters Acquity BEH C8 column (1.7 μm, 50×2.1 mm)]; ¹H NMR (400 MHz, Chloroform-d): δ 8.89 (s, 1H), 8.70 (d, J=1.9 Hz, 1H), 7.82 (d, J=7.4 Hz, 1H), 7.40 (q, J=8.8 Hz, 4H), 5.83 (q, J=8.2 Hz, 1H), 2.74 (s, 3H).

Synthesis of (R)-5-fluoro-N-(2,2,2-trifluoro-1-(4-methoxyphenyl)ethyl)pyridine-3-sulfonamide 138.4: Sulfonamide 138.4 was synthesized from 5-fluoropyridine-3-sulfonyl chloride following the protocol as described in Method O using (R)-2,2,2-trifluoro-1-(4-methoxyphenyl)ethan-1-amine hydrochloride. The compound was purified by column chromatography over silica gel using 20% ethyl acetate in hexane to isolate desired compound 138.4 as yellow gum (400 mg, 58% yield). LCMS: m/z found 365.0 [M+H]⁺, rt=1.82 min (Method 3) [YMC Triart C18 column (3 μm, 33×2.1 mm)].

Synthesis of (R)-5-fluoro-N-methyl-N-(2,2,2-trifluoro-1-(4-methoxyphenyl)ethyl)pyridine-3-sulfonamide: The methylation of intermediate 138.4 was preformed following the protocol as described in Method D. The crude was purified by column chromatography over silica gel using 10% ethyl acetate in hexane to obtain compound Example 221 as a light-yellow sticky gum (80 mg, 97.97% purity). LCMS: m/z found 379.14[M+H]⁺, rt=3.14 min (Method 2) [Waters Acquity BEH C8 column (1.7 μm, 50×2.1 mm)]; ¹H NMR (400 MHz, Chloroform-d): δ 8.88 (s, 1H), 8.68 (d, J=2.4 Hz, 1H), 7.86-7.76 (m, 1H), 7.33 (d, J=8.4 Hz, 2H), 6.92 (d, J=8.8 Hz, 2H), 5.80 (q, J=8.8 Hz, 1H), 3.82 (s, 3H), 2.75 (s, 3H).

Synthesis of (R)-5-fluoro-N-(2,2,2-trifluoro-1-(p-tolyl)ethyl)pyridine-3-sulfonamide 138.5: Sulfonamide 138.5 was synthesized from 5-fluoropyridine-3-sulfonyl chloride following the protocol as described in Method O using (R)-2,2,2-trifluoro-1-(p-tolyl)ethan-1-amine hydrochloride. The compound was purified by column chromatography over silica gel using 20% ethyl acetate in hexane to isolate desired compound 138.5 as a yellow gum (110 mg, 20% yield). LCMS: m/z found 349.0 [M+H]⁺, rt=1.86 min (Method 3) [YMC Triart C18 column (3 μm, 33×2.1 mm)].

Synthesis of (R)-5-fluoro-N-methyl-N-(2,2,2-trifluoro-1-(p-tolyl)ethyl)pyridine-3-sulfonamide: The methylation of intermediate 138.5 was preformed following the protocol as described in Method D. The crude was purified by column chromatography over silica gel using 10% ethyl acetate in hexane to obtain compound Example 222 as a colorless sticky gum (60 mg, 96.08% purity). LCMS: m/z found 363.18 [M+H]⁺, rt=3.25 min (Method 2) [Waters Acquity BEH C8 column (1.7 μm, 50×2.1 mm)]; ¹H NMR (400 MHz, DMSO-d₆): δ 8.98-8.89 (m, 2H), 8.37-8.28 (m, 1H), 7.23 (d, J=8.0 Hz, 2H), 7.18 (d, J=8.0 Hz, 2H), 5.96 (q, J=8.7 Hz, 1H), 2.80 (s, 3H), 2.28 (s, 3H).

Synthesis of (R)-5-fluoro-N-(2,2,2-trifluoro-1-(4-fluorophenyl)ethyl)pyridine-3-sulfonamide 138.6: Sulfonamide 138.6 was synthesized from 5-fluoropyridine-3-sulfonyl chloride following the protocol as described in Method O using (R)-2,2,2-trifluoro-1-(4-fluorophenyl)ethan-1-amine hydrochloride. The compound was purified by column chromatography over silica gel using 20% ethyl acetate in hexane to isolate desired compound 138.6 as yellow gum (160 mg, 24% yield). LCMS: m/z found 353.05 [M+H]⁺, rt=1.82 min (Method 3) [YMC Triart C18 column (3 μm, 33×2.1 mm)].

Synthesis of (R)-5-fluoro-N-methyl-N-(2,2,2-trifluoro-1-(4-fluorophenyl)ethyl)pyridine-3-sulfonamide: The methylation of intermediate 138.6 was preformed following the protocol as described in Method D. The crude was purified by column chromatography over silica gel using 10% ethyl acetate in hexane to obtain compound Example 223 as a light-yellow sticky gum (70 mg, 99.12% purity). LCMS: m/z found 367.2 [M+H]⁺, rt=2.05 min (Method 31) [Waters Acquity BEH C18 column (1.7 μm, 50×2.1 mm)]; ¹H NMR (400 MHz, DMSO) δ 8.98-8.90 (m, 2H), 8.37-8.30 (m, 1H), 7.44 (dd, J=8.7, 5.3 Hz, 2H), 7.24 (t, J=8.8 Hz, 2H), 6.09 (q, J=8.6 Hz, 1H), 2.83 (s, 3H).

Synthesis of (R)-5-fluoro-N-(2,2,2-trifluoro-1-(4-(trifluoromethyl)phenyl)ethyl)pyridine-3-sulfonamide 138.7: Sulfonamide 138.7 was synthesized from 5-fluoropyridine-3-sulfonyl chloride following the protocol as described in Method O using (R)-2,2,2-trifluoro-1-(4-(trifluoromethyl)phenyl)ethan-1-amine hydrochloride. The compound was purified by column chromatography over silica gel using 20% ethyl acetate in hexane to isolate desired compound 138.7 as yellow gum (360 mg, 21% yield). ¹H NMR (400 MHz, DMSO-d₆): δ 9.90-9.80 (brs, 1H), 8.72-8.67 (m, 2H), 7.86 (d, J=7.2 Hz, 1H), 7.75-7.65 (m, 4H), 5.70-7.65 (m, 1H).

Synthesis of (R)-5-fluoro-N-methyl-N-(2,2,2-trifluoro-1-(4-(trifluoromethyl)phenyl)ethyl)pyridine-3-sulfonamide: The methylation of intermediate 1387 was preformed following the protocol as described in Method D. The crude was purified by column chromatography over silica gel using 10% ethyl acetate in hexane to obtain compound Example 224 as a yellow gum (200 mg, 99.07% purity, 65% yield). ¹H NMR (400 MHz, DMSO-d₆): δ 9.0-8.9 (m, 2H), 8.36-8.31 (m, 1H), 7.80 (d, J=8.2 Hz, 2H), 7.61 (d, J=8.1 Hz, 2H), 6.28-6.21 (m, 1H), 2.86 (s, 3H).

### Example 225

### (R)-5-cyano-N-methyl-N-(2,2,2-trifluoro-1-(p-tolyl)ethyl)pyridine-3-sulfonamide

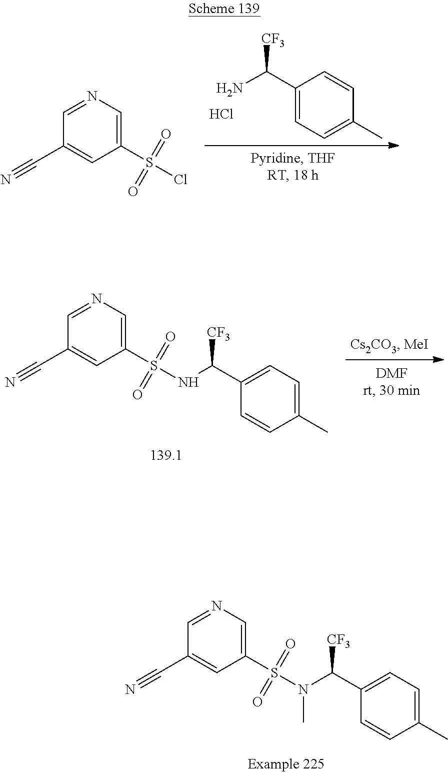

Synthesis of (R)-5-cyano-N-(2,2,2-trifluoro-1-(p-tolyl)ethyl)pyridine-3-sulfonamide 139.1: Sulfonamide 139.1 was synthesized from 5-fluoropyridine-3-sulfonyl chloride following the protocol as described in Method O using (R)-2,2,2-trifluoro-1-(p-tolyl)ethan-1-amine hydrochloride. The compound was purified by column chromatography over silica gel using 20% ethyl acetate in hexane to isolate desired compound 139.1 as a yellow oil (160 mg, 24% yield). LCMS: m/z found 356.0 [M+H]⁺, rt=1.85 min (Method 3) [YMC Triart C18 column (3 μm, 33×2.1 mm)].

Synthesis of (R)-5-cyano-N-methyl-N-(2,2,2-trifluoro-1-(p-tolyl)ethyl)pyridine-3-sulfonamide: The methylation of intermediate 139.1 was performed following the protocol as described in Method D. The crude was purified by column chromatography over silica gel using 10% ethyl acetate in hexane to obtain compound Example 225 as a light brown sticky gum (25 mg, 99.89% purity). LCMS: m/z found 370.20[M+H]⁺, rt=3.41 min (Method 2) [Waters Acquity BEH C8 column (1.7 μm, 50×2.1 mm)]; ¹H NMR (400 MHz, DMSO-d₆) δ 9.31 (s, 2H), 8.86 (s, 1H), 7.26-7.15 (m, 4H), 5.96 (q, J=8.6 Hz, 1H), 2.82 (s, 3H), 2.28 (s, 3H).

### Example 226

### (R)—N-methyl-N-(2,2,2-trifluoro-1-(p-tolyl)ethyl)pyrimidine-5-sulfonamide

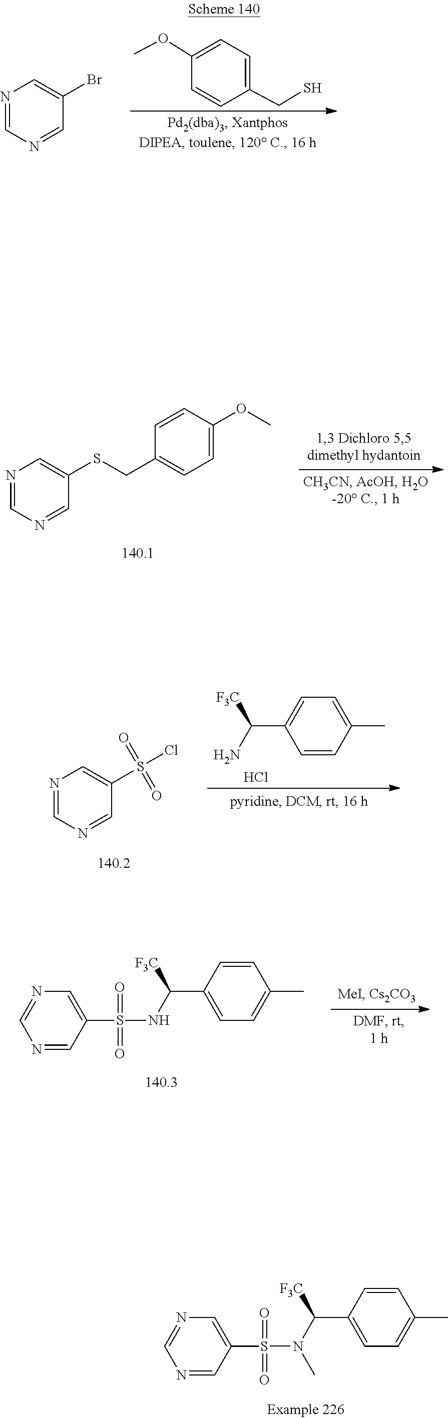

Synthesis of 5-((4-methoxybenzyl)thio)pyrimidine 140.1: Intermediate 140.1 was synthesized from 5-bromopyrimidine following a method analogous to that described in Method I using (4-methoxyphenyl)methanethiol. The crude was purified by column chromatography over silica gel using 20% EA-Hex to get compound 140.1 as a yellow oil (3.5 g, 48% yield). LCMS: m/z found 233.3 [M+H]⁺, rt=1.81 min (Method 31) [Waters Acquity BEH C18 column (1.7 μm, 50×2.1 mm)].

Synthesis of pyrimidine-5-sulfonyl chloride 140.2: Sulfonyl chloride 140.2 was synthesized from Intermediate 140.1 following the procedure described in Method T. The crude sulfonyl chloride was used immediately in the forwarding step without further purification.

Synthesis of (R)—N-(2,2,2-trifluoro-1-(p-tolyl)ethyl)pyrimidine-5-sulfonamide 140.3: Intermediate 140.3 was synthesized from Sulfonyl chloride 140.2 following the protocol as described in Method O using (R)-2,2,2-trifluoro-1-(p-tolyl)ethan-1-amine hydrochloride. The compound was purified by column chromatography over silica gel using 35% ethyl acetate in hexane and intermediate 140.3 was isolated as a yellow gum (100 mg, 14% yield). LCMS: m/z found 332.0 [M+H]⁺, rt=1.81 min (Method 31) [Waters Acquity BEH C18 column (1.7 μm, 50×2.1 mm)].

Synthesis of (R)—N-methyl-N-(2,2,2-trifluoro-1-(p-tolyl)ethyl)pyrimidine-5-sulfonamide: The methylation of intermediate 140.3 was preformed following the protocol as described in Method D. The crude was purified by column chromatography over silica gel using 10% ethyl acetate in hexane to obtain compound Example 226 as a colorless sticky gum (40 mg, 98.55% purity, 38% yield). ¹H NMR (400 MHz, Chloroform-d) δ 9.38 (s, 1H), 9.11 (d, J=2.3 Hz, 2H), 7.30 (d, J=7.6 Hz, 2H), 7.22 (d, J=8.0 Hz, 2H), 5.82 (q, J=8.4 Hz, 1H), 2.77 (s, 3H), 2.36 (s, 3H).

### Example 227

### (R)—N-(1-(4-chlorophenyl)-2,2,2-trifluoroethyl)-5-cyano-N-(methyl-d3)pyridine-3-sulfonamide

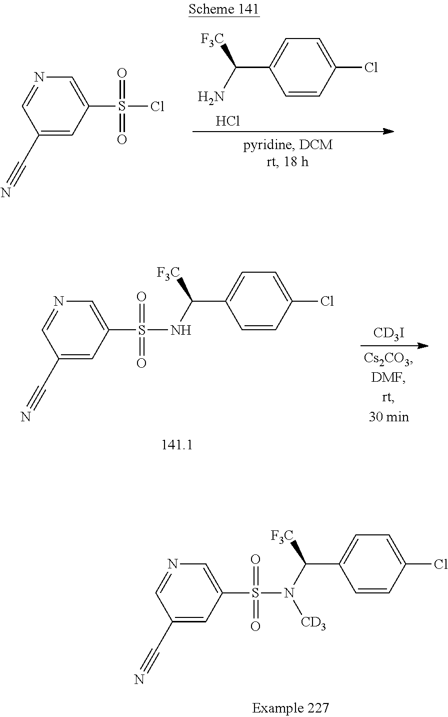

Synthesis of (R)—N-(1-(4-chlorophenyl)-2,2,2-trifluoroethyl)-5-cyanopyridine-3-sulfonamide 141.1: Sulfonamide 141.1 was synthesized from 5-cyanopyridine-3-sulfonyl chloride following the protocol as described in Method O using (R)-1-(4-chlorophenyl)-2,2,2-trifluoroethan-1-amine hydrochloride. The compound was purified by column chromatography over silica gel using 20% ethyl acetate in hexane to isolate desired compound 141.1 as a yellow gum (300 mg, 32% yield). ¹H NMR (400 MHz, DMSO-d₆): δ 9.15 (d, J=1.5 Hz, 1H), 9.02 (d, J=1.9 Hz, 1H), 8.44 (t, J=1.5 Hz, 1H), 7.52 (d, J=8.3 Hz, 1H), 7.48-7.40 (m, 2H), 7.35 (d, J=8.4 Hz, 2H), 5.56-5.48 (m, 1H).

Synthesis of (R)—N-(1-(4-chlorophenyl)-2,2,2-trifluoroethyl)-5-cyano-N-(methyl-d3)pyridine-3-sulfonamide: The alkylation of intermediate 141.1 was performed following the protocol as described in Method D using iodomethane-d3. The crude was purified by column chromatography over silica gel using 10% ethyl acetate in hexane to obtain compound Example 227 as a white solid (100 mg, 99.09% purity, 38% yield). ¹H NMR (400 MHz, DMSO-d₆): δ 9.32 (d, J=1.8 Hz, 2H), 8.89 (s, 1H), 7.49 (d, J=8.5 Hz, 2H), 7.39 (d, J=8.4 Hz, 2H), 6.10 (q, J=8.5 Hz, 1H).

### Example 228

### (R)—N-(1-(4-chlorophenyl)-2,2,2-trifluoroethyl)-3,6-dimethoxy-N-methylpyridazine-4-sulfonamide

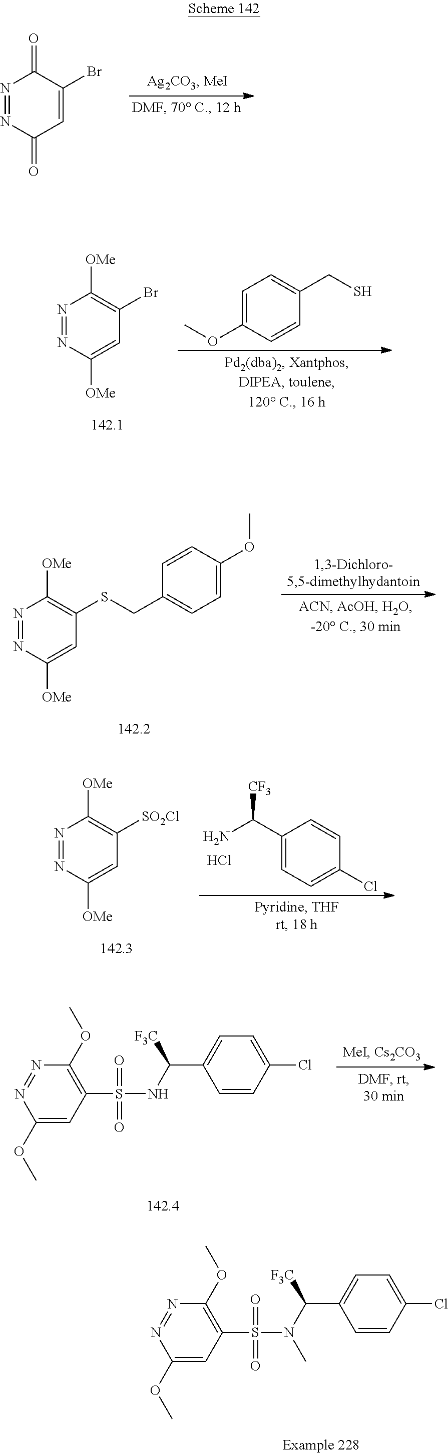

Synthesis of 4-bromo-3,6-dimethoxypyridazine 142.1: Method Z: O-methylation: To a stirred solution of 4-bromopyridazine-3,6-dione (1.0 g, 5.23 mmol) in DMF (10 mL) was added Ag₂CO₃ (4.3 g, 15.70 mmol) followed by Mel (3.3 mL, 52.35 mmol). The reaction mixture was heated at 90° C. for 12 h. The reaction mixture was filtered, and the filtrate was evaporated under reduced pressure. The crude product was purified by column chromatography over silica gel using 30% ethyl acetate in hexane to obtain compound 142.1 as brown solid (400 mg, 32% yield). ¹H NMR (400 MHz, DMSO-d₆): δ 7.81 (s, 1H), 3.75 (s, 3H), 3.59 (s, 3H).

Synthesis of 3,6-dimethoxy-4-((4-methoxybenzyl)thio)pyridazine 142.2: Intermediate 142.2 was synthesized from compound 142.1 following a method analogous to that described in Method I using (4-methoxyphenyl)methanethiol. The crude was purified by column chromatography over silica gel using 20% ethyl acetate in hexane to get compound 142.2 as yellow gum (1.0 g, 66% yield). ¹H NMR (400 MHz, DMSO-d₆): δ 7.56-7.34 (m, 2H), 7.0-6.8 (m, 2H), 6.72 (s, 1H), 4.21 (s, 2H), 3.78 (s, 3H), 3.73 (s, 3H), 3.46 (s, 3H).

Synthesis of 3,6-dimethoxypyridazine-4-sulfonyl chloride 142.3: Sulfonyl chloride 142.3 was synthesized from Intermediate 142.2 following the procedure described in Method T. The crude sulfonyl chloride was used immediately in the forwarding step without further purification.

Synthesis of (R)—N-(1-(4-chlorophenyl)-2,2,2-trifluoroethyl)-3,6-dimethoxypyridazine-4-sulfonamide 142.4: Intermediate 142.4 was synthesized from sulfonyl chloride 142.3 following the protocol as described in Method O using (R)-1-(4-chlorophenyl)-2,2,2-trifluoroethan-1-amine hydrochloride. The compound was purified by column chromatography over silica gel using 35% ethyl acetate in hexane and intermediate 142.4 was isolated as yellow gum (200 mg, 14% yield). ¹H NMR (400 MHz, DMSO-d₆): δ 9.91-9.87 (m, 1H), 7.53 (d, J=8.2 Hz, 2H), 7.45 (d, J=8.6 Hz, 2H), 7.18 (s, 1H), 5.43-5.39 (m, 1H), 3.61 (s. 3H), 3.45 (s, 3H).

Synthesis of (R)—N-(1-(4-chlorophenyl)-2,2,2-trifluoroethyl)-3,6-dimethoxy-N-methylpyridazine-4-sulfonamide: The methylation of intermediate 142.4 was preformed following the protocol as described in Method D. The crude was purified by column chromatography over silica gel using 10% ethyl acetate in hexane to obtain compound Example 228 as an off-white solid (30 mg, 99.81% purity, 25% yield). ¹H NMR (400 MHz, DMSO-d₆): δ 7.56-7.52 (m, 2H), 7.47-7.42 (m, 2H), 7.38 (s, 1H), 6.0-5.92 (m, 1H), 3.17 (s, 3H), 3.57 (s, 3H), 2.96 (s, 3H).

### Example 229

### (R)—N-(1-(4-chlorophenyl)-2,2,2-trifluoroethyl)-6-methoxy-N-methylpyridazine-4-sulfonamide

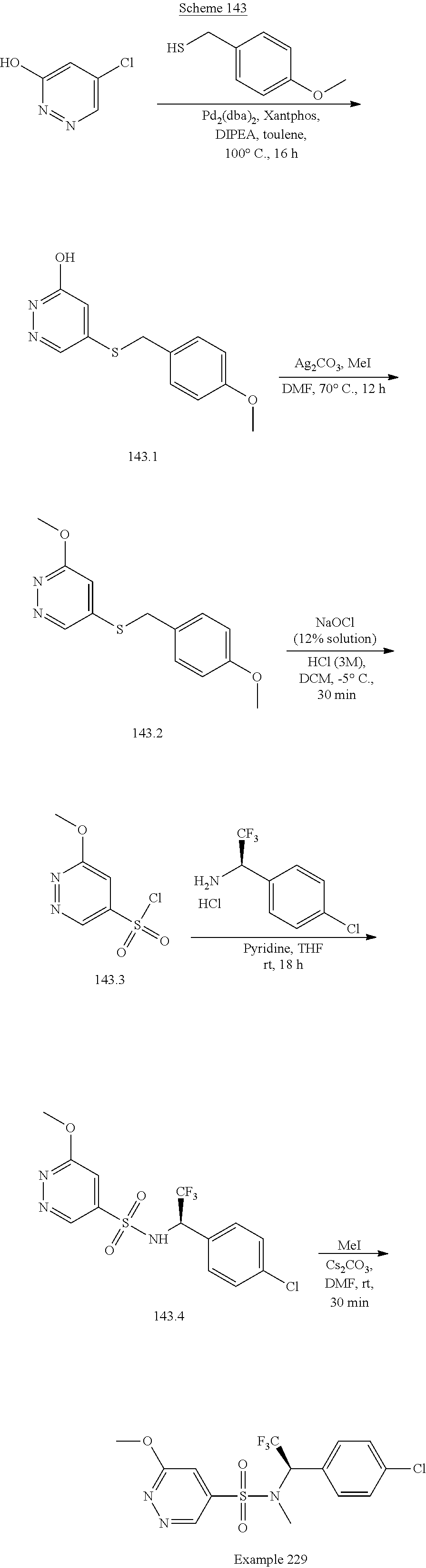

Synthesis of 5-((4-methoxybenzyl)thio)pyridazin-3-ol 143.1: Intermediate 143.1 was synthesized from 5-chloropyridazin-3-ol following a method analogous to that described in Method I using (4-methoxyphenyl)methanethiol. The crude was triturated with 10% ethyl acetate in hexane to get compound 143.1 as brown solid (1.0 g, 66% yield). ¹H NMR (400 MHz, DMSO-d₆): δ 9.50 (s, 1H), 7.31 (d, J=2.0 Hz, 1H), 7.35 (d, J=8.5 Hz, 2H), 6.91 (d, J=8.5 Hz, 2H), 6.65 (s, 1H), 4.28 (s, 2H), 3.73 (s, 3H).

Synthesis of 3-methoxy-5-((4-methoxybenzyl)thio)pyridazine 143.2: Intermediate 143.2 was synthesized from intermediate 143.1 following the procedure described in Method Z. The crude product was triturated with 20% ethyl acetate and used in forwarding step without further purification (2.0 g, 51% yield). ¹H NMR (400 MHz, DMSO-d₆): δ 7.78 (d, J=2.0 Hz, 1H), 7.35 (d, J=8.5 Hz, 2H), 6.91 (d, J=8.6 Hz, 2H), 6.73 (d, J=2.2 Hz, 1H), 4.29 (s, 2H), 3.73 (s, 3H), 3.55 (s, 3H).

Synthesis of 6-methoxypyridazine-4-sulfonyl chloride 143.3: Sulfonyl chloride 143.3 was synthesized from Intermediate 143.2 following the procedure described in Method T. The crude sulfonyl chloride was used immediately in the forwarding step without further purification.

Synthesis of (R)—N-(1-(4-chlorophenyl)-2,2,2-trifluoroethyl)-6-methoxypyridazine-4-sulfonamide 143.4: Intermediate 143.4 was synthesized from sulfonyl chloride 143.3 following the protocol as described in Method O using (R)-1-(4-chlorophenyl)-2,2,2-trifluoroethan-1-amine hydrochloride. The compound was purified by column chromatography over silica gel using 35% ethyl acetate in hexane and intermediate 143.4 was isolated as a yellow gum (70 mg, 5% yield). ¹H NMR (400 MHz, DMSO-d₆): δ 10.02 (s, 1H), 7.93 (d, J=2.1 Hz, 1H), 7.50 (d, J=8.4 Hz, 2H), 7.42 (d, J=8.5 Hz, 2H), 7.03 (d, J=2.1 Hz, 1H), 5.57-5.53 (m, 1H), 3.58 (s, 3H).

Synthesis of (R)—N-(1-(4-chlorophenyl)-2,2,2-trifluoroethyl)-6-methoxy-N-methylpyridazine-4-sulfonamide: The methylation of intermediate 143.4 was preformed following the protocol as described in Method D. The crude was purified by column chromatography over silica gel using 10% ethyl acetate in hexane to obtain compound Example 229 as a yellow sticky gum (30 mg, 93.58% purity, 42% yield). ¹H NMR (400 MHz, DMSO-d₆): δ 8.28 (d, J=2.2 Hz, 1H), 7.54 (d, J=8.6 Hz, 2H), 7.49 (d, J=8.6 Hz, 2H), 7.41 (d, J=2.1 Hz, 1H), 6.15-6.08 (m, 1H), 3.69 (s, 3H), 2.86 (s, 3H).

### Example 230

### (R)—N-(1-(4-chlorophenyl)-2,2,2-trifluoroethyl)-N-methylimidazo[1,2-a]pyrimidine-3-sulfonamide

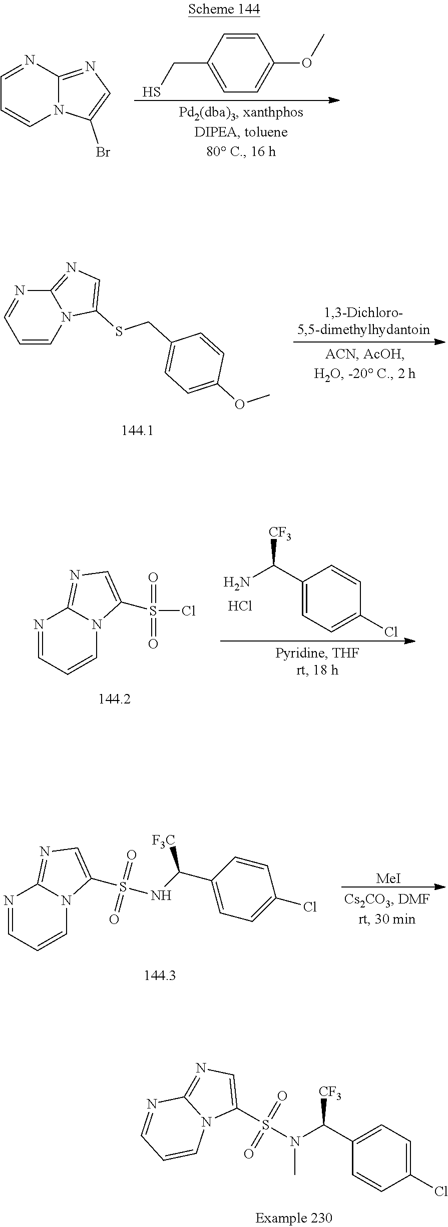

Synthesis of 3-((4-methoxybenzyl)thio)imidazo[1,2-a]pyrimidine 144.1: Intermediate 144.1 was synthesized from 3-bromoimidazo[1,2-a]pyrimidine following a method analogous to that described in Method AA using (4-methoxyphenyl)methanethiol. The reaction was then filtered through sintered funnel and the residue was collected, concentrated to a residue. The crude was purified by column chromatography over silica gel using 1:4 ethyl acetate-hexane to yield the intermediate 144.1 as brown liquid (2.0 g, 80% yield). ¹H NMR (400 MHz, Chloroform-d): δ 8.37 (d, J=6.7 Hz, 1H), 8.24 (s, 1H), 7.49 (s, 1H), 7.31 (d, J=8.5 Hz, 2H), 6.85 (d, J=8.6 Hz, 3H), 4.21 (s, 2H), 3.78 (s, 3H).

Synthesis of imidazo[1,2-a]pyrimidine-3-sulfonyl chloride 144.2: Sulfonyl chloride 144.2 was synthesized from intermediate 144.1 following the procedure described in Method AB. The crude sulfonyl chloride was used immediately in the forwarding step without further purification.

Synthesis of (R)—N-(1-(4-chlorophenyl)-2,2,2-trifluoroethyl)imidazo[1,2-a]pyrimidine-3-sulfonamide 144.3: Intermediate 144.3 was synthesized from sulfonyl chloride 144.2 following the protocol as described in Method AC using (R)-1-(4-chlorophenyl)-2,2,2-trifluoroethan-1-amine hydrochloride, The compound was purified by column chromatography over silica gel using 3:7 ethyl acetate:hexane to provide compound 144.3 as off white solid (300 mg, 22% yield).

Synthesis of (R)—N-(1-(4-chlorophenyl)-2,2,2-trifluoroethyl)-N-methylimidazo[1,2-a]pyrimidine-3-sulfonamide: The methylation of intermediate 144.3 was preformed following the protocol as described in Method AD. The crude was purified by column chromatography over silica gel using 1:4 ethyl acetate-hexane to obtain compound Example 230 as a colorless gum (45 mg, 99.09% purity, 15% yield). LCMS: m/z found 405.20 [M+H]⁺, rt=3.33 min (Method 2) [Waters Acquity BEH C8 column (1.7 μm, 50×2.1 mm)]; ¹H NMR (400 MHz, DMSO-d₆): δ 9.17 (d, J=6.8 Hz, 1H), 8.89-8.80 (m, 1H), 8.47 (s, 1H), 7.49-7.34 (m, 5H), 6.17 (q, J=8.3 Hz, 1H), 2.83 (s, 3H).

### Example 231 and Example 232

### (R)—N-(1-(4-chlorophenyl)-2,2,2-trifluoroethyl)-N-methylimidazo[1,2-a]pyrazine-2-sulfonamide (Example 231) and (R)—N-ethyl-N-(2,2,2-trifluoro-1-(4-fluorophenyl)ethyl)imidazo[1,2-a]pyrazine-2-sulfonamide (Example 232)

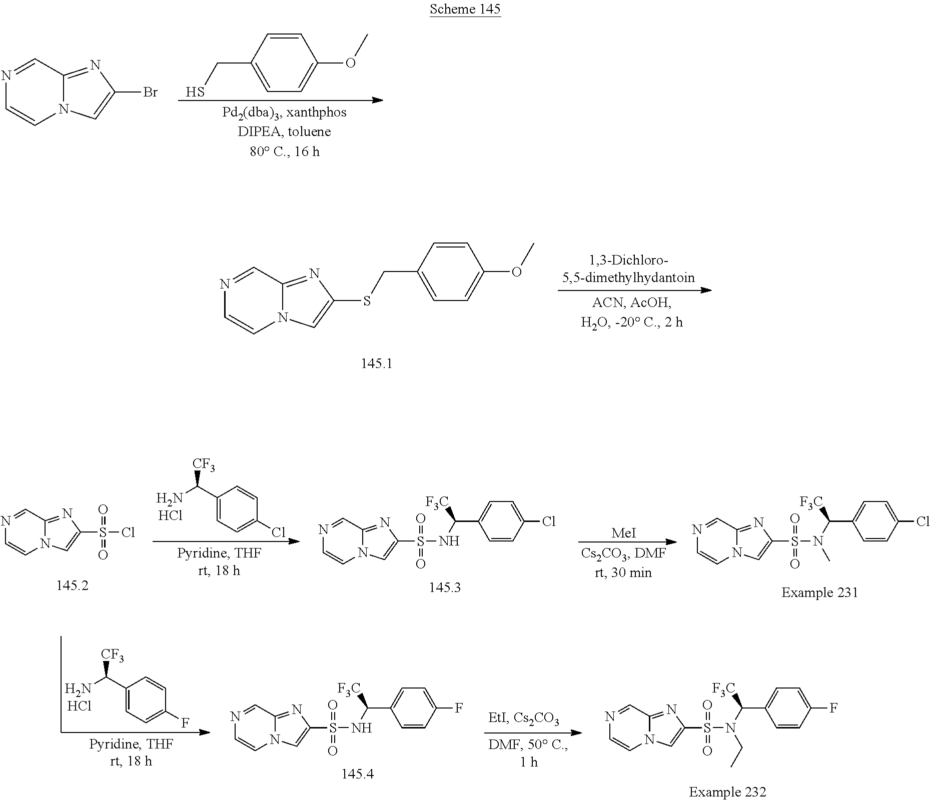

Synthesis of 2-((4-methoxybenzyl)thio)imidazo[1,2-a]pyrazine 145.1: Intermediate 145.1 was synthesized from 2-bromoimidazo[1,2-a]pyrazine following a method analogous to that described in Method AA using (4-methoxyphenyl)methanethiol. The reaction was then filtered through sintered funnel and the residue was collected, concentrated to a residue. The crude was purified by column chromatography over silica gel using 1:4 ethyl acetate-hexane to yield the intermediate 145.1 as a brown solid (600 mg, 87% yield). LCMS: m/z found 272.23 [M+H]⁺, rt=1.61 min (Method 1) [Waters Acquity BEH C8 column (1.7 μm, 50×2.1 mm)].

Synthesis of imidazo[1,2-a]pyrazine-2-sulfonyl chloride 145.2: Sulfonyl chloride 145.2 was synthesized from intermediate 145.1 following the procedure described in Method AB. The crude sulfonyl chloride was used immediately in the forwarding step without further purification.

Synthesis of (R)—N-(1-(4-chlorophenyl)-2,2,2-trifluoroethyl)imidazo[1,2-a]pyrazine-2-sulfonamide 145.3: Intermediate 145.3 was synthesized from sulfonyl chloride 145.2 following the protocol as described in Method AC using (R)-1-(4-chlorophenyl)-2,2,2-trifluoroethan-1-amine hydrochloride. The compound was purified by column chromatography over silica gel using 3:7 ethyl acetate:hexane to provide compound 145.3 as an off-white solid (300 mg, 34% yield). LCMS: m/z found 391.5 [M+H]⁺, rt=1.78 min (Method 31) [Waters Acquity BEH C18 column (1.7 μm, 50×2.1 mm)].

Synthesis of (R)—N-(1-(4-chlorophenyl)-2,2,2-trifluoroethyl)-N-methylimidazo[1,2-a]pyrazine-2-sulfonamide: The methylation of intermediate 145.3 was preformed following the protocol as described in Method AD. The crude was purified by column chromatography over silica gel using 1:4 ethyl acetate-hexane to obtain compound Example 231 as an off-white sticky solid (80 mg, 20% yield). LCMS: m/z found 405.20 [M+H]⁺, rt=2.87 min (Method 2) [Waters Acquity BEH C8 column (1.7 μm, 50×2.1 mm)]; ¹H NMR (400 MHz, DMSO-d₆): δ 9.22 (s, 1H), 8.69 (s, 1H), 8.62 (dd, J=1.6, 4.6 Hz, 1H), 8.07 (d, J=4.6 Hz, 1H), 7.47-7.37 (m, 4H), 5.96 (q, J=8.5 Hz, 1H), 2.78 (s, 3H).

Synthesis of (R)—N-(2,2,2-trifluoro-1-(4-fluorophenyl)ethyl)imidazo[1,2-a]pyrazine-2-sulfonamide 145.4: Intermediate 145.4 was synthesized from sulfonyl chloride 145.2 following the protocol as described in Method AC using (R)-1-(4-fluorophenyl)-2,2,2-trifluoroethan-1-amine hydrochloride. The compound was purified by column chromatography over silica gel using 3:7 ethyl acetate:hexane to provide compound 145.4 as off white solid (100 mg, 23% yield). LCMS: m/z found 375.13 [M+H]⁺, rt=1.70 min (Method 32) [Waters Acquity BEH C18 column (1.7 μm, 50×2.1 mm)].

Synthesis of (R)—N-ethyl-N-(2,2,2-trifluoro-1-(4-fluorophenyl)ethyl)imidazo[1,2-a]pyrazine-2-sulfonamide: The methylation of intermediate 145.4 was preformed following the protocol as described in Method AD. The crude was purified by column chromatography over silica gel using 1:4 ethyl acetate-hexane to obtain compound Example 232 as a colorless sticky gum (25 mg, 99.81% purity, 20% yield). LCMS: m/z found 403.28 [M+H]⁺, rt=2.87 min (Method 2) [Waters Acquity BEH C8 column (1.7 μm, 50×2.1 mm)]; ¹H NMR (400 MHz, DMSO-d₆): δ 9.22 (s, 1H), 8.66 (s, 1H), 8.61 (d, J=4.3 Hz, 1H), 8.06 (d, J=4.6 Hz, 1H), 7.48 (dd, J=5.3, 8.5 Hz, 2H), 7.16 (t, J=8.7 Hz, 2H), 5.91 (q, J=8.6 Hz, 1H), 3.47-3.33 (m, 1H), 3.29-3.19 (m, 1H), 1.02 (t, J=7.0 Hz, 3H).

### Example 233 and Example 234

### (R)-3-chloro-N-methyl-N-(2,2,2-trifluoro-1-(4-(trifluoromethyl)phenyl)ethyl)imidazo[1,2-a]pyridine-6-sulfonamide (Example 233) and (R)—N-methyl-N-(2,2,2-trifluoro-1-(4-(trifluoromethyl)phenyl)ethyl)imidazo[1,2-a]pyridine-6-sulfonamide (Example 234)

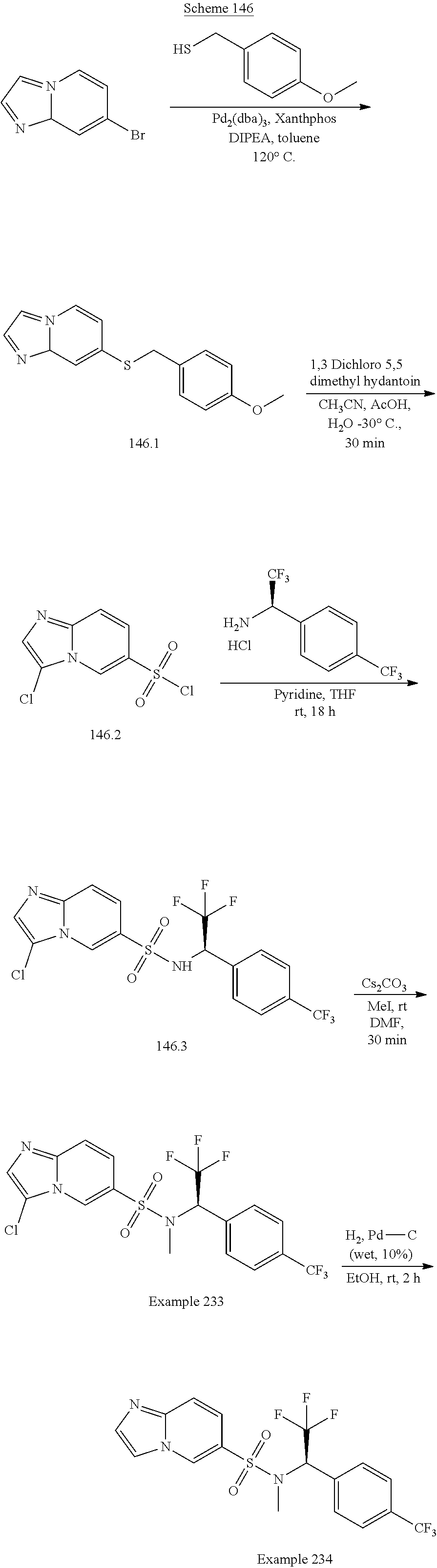

Synthesis of 6-((4-methoxybenzyl)thio)imidazo[1,2-a]pyridine 146.1: Intermediate 146.1 was synthesized from 6-bromoimidazo[1,2-a]pyridine following a method analogous to that described in Method AA using (4-methoxyphenyl)methanethiol. The reaction was then filtered through sintered funnel and the residue was collected, concentrated to a residue. The crude was purified by column chromatography over silica gel using 1:4 ethyl acetate-hexane to yield the intermediate 3.1 as brown gum (2.2 g, 80% yield). LCMS: m/z found 271.0 [M+H]⁺, rt=1.23 min (Method 3) [YMC Triart C18 column (3 μm, 33×2.1 mm)].

Synthesis of 3-chloroimidazo[1,2-a]pyridine-6-sulfonyl chloride 146.2: Sulfonyl chloride 146.2 was synthesized from intermediate 146.1 following the procedure described in Method AB. The crude sulfonyl chloride was used immediately in the forwarding step without further purification.

Synthesis of (R)-3-chloro-N-(2,2,2-trifluoro-1-(4-(trifluoromethyl)phenyl)ethyl)imidazo[1,2-a]pyridine-6-sulfonamide 146.3: Intermediate 146.3 was synthesized from sulfonyl chloride 146.2 following the protocol as described in Method AC using (R)-2,2,2-trifluoro-1-(4-(trifluoromethyl)phenyl)ethan-1-amine hydrochloride. The compound was purified by column chromatography over silica gel using 3:7 ethyl acetate:hexane to provide compound 146.3 as colorless gum (150 mg, 9% yield). LCMS: m/z found 458.0 [M+H]⁺, rt=1.76 min (Method 3) [YMC Triart C18 column (3 μm, 33×2.1 mm)].

Synthesis of (R)-3-chloro-N-methyl-N-(2,2,2-trifluoro-1-(4-(trifluoromethyl)phenyl)ethyl)imidazo[1,2-a]pyridine-6-sulfonamide: The methylation of intermediate 146.3 was preformed following the protocol as described in Method AD. The crude was purified by column chromatography over silica gel using 1:4 ethyl acetate-hexane to obtain compound Example 233 as a yellow sticky gum (75 mg, 98.62% purity, 48% yield). LCMS: m/z found 472.20 [M+H]⁺, rt=3.06 min (Method 2) [Waters Acquity BEH C8 column (1.7 μm, 50×2.1 mm)]; ¹H NMR (400 MHz, DMSO-d₆): δ 8.81-8.75 (m, 1H), 7.93 (s, 1H), 7.81 (d, J=9.7 Hz, 1H), 7.76 (d, J=8.3 Hz, 2H), 7.69-7.59 (m, 3H), 6.33 (q, J=8.6 Hz, 1H), 2.88 (s, 3H).

(R)—N-methyl-N-(2,2,2-trifluoro-1-(4-(trifluoromethyl)phenyl)ethyl)imidazo[1,2-a]pyridine-6-sulfonamide: The hydrogenation of Example 233 was performed following the protocol as described in Method AE. The crude was purified by column chromatography over silica gel using 1:4 ethyl acetate-hexane to obtain compound Example 234 as a yellow sticky gum (70 mg, 99.82% purity). LCMS: m/z found 438.24 [M+H]⁺, rt=2.80 min (Method 2) [Waters Acquity BEH C8 column (1.7 μm, 50×2.1 mm)]; ¹H NMR (400 MHz, DMSO-d₆): δ 9.36-9.30 (m, 1H), 8.13 (s, 1H), 7.83-7.70 (m, 4H), 7.66 (d, J=8.1 Hz, 2H), 7.52 (dd, J=1.8, 9.6 Hz, 1H), 6.21 (q, J=8.5 Hz, 1H), 2.80 (s, 3H).

### Example 235

### (R)—N-methyl-N-(2,2,2-trifluoro-1-(4-(trifluoromethyl)phenyl)ethyl)imidazo[1,2-a]pyridine-3-sulfonamide

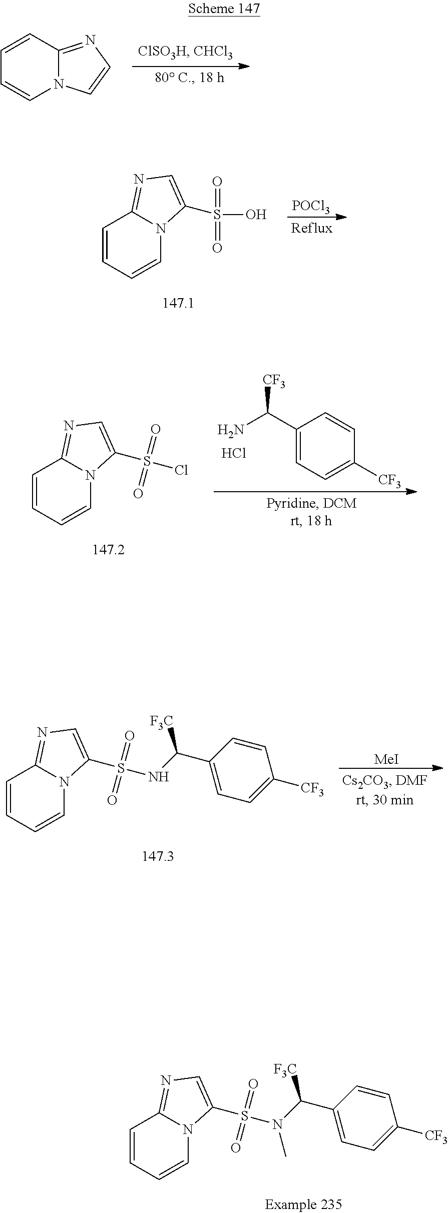

Synthesis of imidazo[1,2-a]pyridine-3-sulfonic acid 147.1: To a stirred suspension of compound imidazo[1,2-a]pyridine (1 g, 8.46 mmol) in CHCl₃ (25 mL) a solution of chlorosulfonic acid (1.7 ml, 25.39 mmol) in CHCl₃ (25 mL) was added dropwise at 0° C. The resultant mixture was refluxed for 18 h. The reaction mixture was cooled to ambient temperature and concentrated under reduced pressure to afford pale brown sticky liquid which was solidified with ethanol and diethylether to afford off white solid. It was filtered and dried under vacuum to afford compound 147.1 (850 mg, 50% yield). The crude solid compound was subjected to next step without further purification.

Synthesis of imidazo[1,2-a]pyridine-3-sulfonyl chloride 147.2: Compound 147.1 (1.60 g, 8.07 mmol) was refluxed with POCl₃ (50 ml) for 18 h. The reaction mass was cooled, concentrated under reduced pressure, and partitioned between DCM (250 ml) and water (150 ml). The organic layer was separated, dried over anhydrous MgSO4, and concentrated under reduced pressure to afford crude compound (1.70 g) which was used for next step reaction without further purification.

Synthesis of (R)—N-(2,2,2-trifluoro-1-(4-fluorophenyl)ethyl)imidazo[1,2-a]pyridine-3-sulfonamide 147.3: Intermediate 147.3 was synthesized from sulfonyl chloride 147.2 following the protocol as described in Method AC using (R)-2,2,2-trifluoro-1-(4-(trifluoromethyl)phenyl)ethan-1-amine hydrochloride. Crude compound was purified by column chromatography using 30% ethyl acetate in hexane as eluent to afford desire product 147.3 as an off-white solid (50 mg, 11% yield). LCMS: m/z found 424.29 (M+H)+, rt=1.86 min (Method 1) [Waters Acquity BEH C18 column (1.7 μm, 50×2.1 mm)].

Synthesis of (R)—N-ethyl-N-(2,2,2-trifluoro-1-(4-fluorophenyl)ethyl)imidazo[1,2-a]pyridine-3-sulfonamide: The methylation of intermediate 147.3 was preformed following the protocol as described in Method AD. The crude was purified by column chromatography over silica gel using 1:5 ethyl acetate-hexane to obtain compound Example 235 as an off-white solid (15 mg, 96.03% purity, 25% yield). LCMS: m/z found (438.22 [M+H]⁺) rt=3.02 min (Method 2) [Waters Acquity BEH C18 column (1.7 μm, 50×2.1 mm)]. ¹H NMR (400 MHz, DMSO): δ 8.72 (d, J=6.8 Hz, 1H), 8.32 (s, 1H), 7.84 (d, J=9.0 Hz, 1H), 7.76 (d, J=8.2 Hz, 2H), 7.66-7.56 (m, 3H), 7.23 (t, J=6.9 Hz, 1H), 6.28 (q, J=8.4 Hz, 1H), 2.84 (s, 3H).

### Example 236

### (R)—N-methyl-N-(2,2,2-trifluoro-1-(4-(trifluoromethyl)phenyl)ethyl)imidazo[1,2-a]pyridine-7-sulfonamide

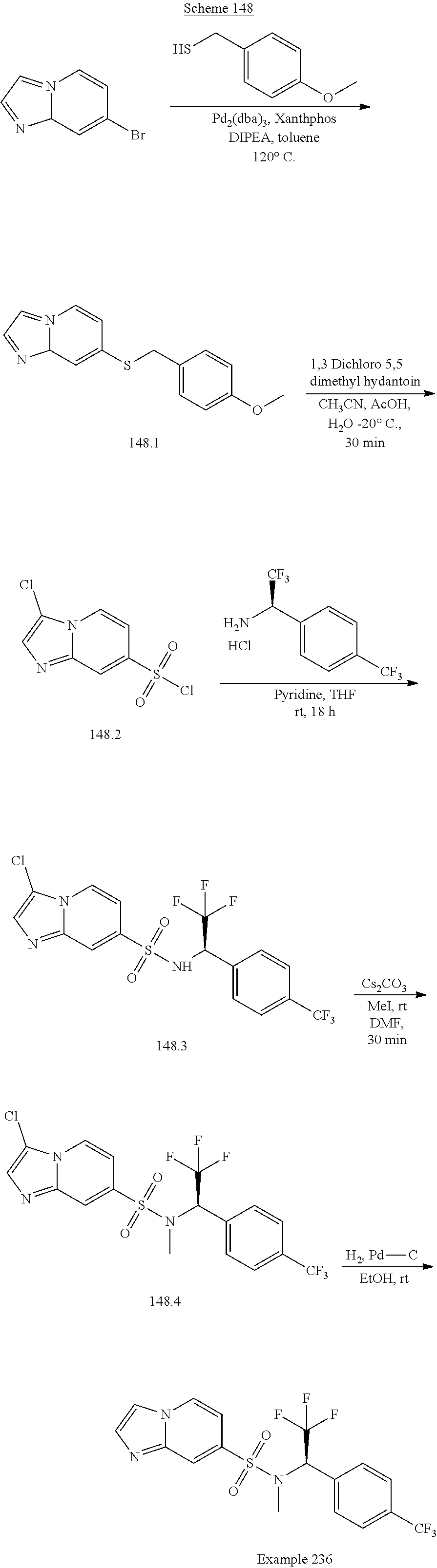

Synthesis of 7-((4-methoxybenzyl)thio)imidazo[1,2-a]pyridine 148.1: Intermediate 148.1 was synthesized following Buchwald protocol as described in method Method AA using (4-methoxyphenyl)methanethiol. The crude residue was purified by column chromatography over silica gel using 50% ethyl acetate in hexane to obtain desired compound 148.1 (500 mg, 72% yield) as yellow sticky gum. LCMS: m/z found 271 [M+H+], rt=2.72 min (Method 2) [Waters Acquity BEH C8 column (1.7 μm, 50×2.1 mm)].

Synthesis of 3-chloroimidazo[1,2-a]pyridine-7-sulfonyl chloride 148.2: Sulfonyl chloride 148.2 was synthesized following the protocol as described in Method AB. The crude sulfonyl chloride 148.2 was used for next step sulfonamidation reaction without any purification.

Synthesis of (R)-3-chloro-N-(2,2,2-trifluoro-1-(4-(trifluoromethyl)phenyl)ethyl)imidazo[1,2-a]pyridine-7-sulfonamide 148.3: Intermediate 148.3 was synthesized following the protocol as described in Method AC using (R)-2,2,2-trifluoro-1-(4-(trifluoromethyl)phenyl)ethan-1-amine hydrochloride. The crude was purified by column chromatography over silica gel using 30% ethyl acetate in hexane to obtain desired compound 148.3 (300 mg, 52% yield) as a colorless sticky gum. LCMS: m/z found 458.0[M+H⁺, rt=1.72 min (Method 3) [YMC Triart C18 column (3 μm, 33×2.1 mm)].

Synthesis of (R)-3-chloro-N-methyl-N-(2,2,2-trifluoro-1-(4-(trifluoromethyl)phenyl)ethyl)imidazo[1,2-a]pyridine-7-sulfonamide 148.4: The methylation of intermediate 148.3 was performed following the protocol as described in Method AD. The crude was purified by column chromatography over silica gel using 1:5 ethyl acetate-hexane to obtain compound 148.4 as off white solid (100 mg, 96.03% purity, 25% yield). LCMS: m/z found 472.20 [M+H]⁺, rt=1.88 min (Method 3) [YMC Triart C18 column (3 μm, 33×2.1 mm)].

Synthesis of (R)—N-methyl-N-(2,2,2-trifluoro-1-(4-(trifluoromethyl)phenyl)ethyl)imidazo[1,2-a]pyridine-7-sulfonamide: The hydrogenation of compound 148.3 was performed following the protocol as described in Method AE. The crude was purified by column chromatography over silica gel using 1:4 ethyl acetate-hexane to obtain compound Example 236 as an off-white solid (25 mg, 99.23% purity, 45% yield). LCMS: m/z found 438.22 [M+H]⁺, rt=2.83 min (Method 2) [Waters Acquity BEH C18 column (1.7 μm, 50×2.1 mm)]. ¹H NMR (400 MHz, DMSO): δ 8.75 (d, J=7.2 Hz, 1H), 8.21-8.15 (m, 2H), 7.86 (s, 1H), 7.76 (d, J=8.3 Hz, 2H), 7.66 (d, J=8.1 Hz, 2H), 7.27 (dd, J=7.2, 1.6 Hz, 1H), 6.26 (q, J=8.4 Hz, 1H), 2.82 (s, 3H).

### Example 237

### (R)—N-ethyl-N-(2,2,2-trifluoro-1-(4-(trifluoromethyl)phenyl)ethyl)imidazo[1,2-a]pyridine-3-sulfonamide

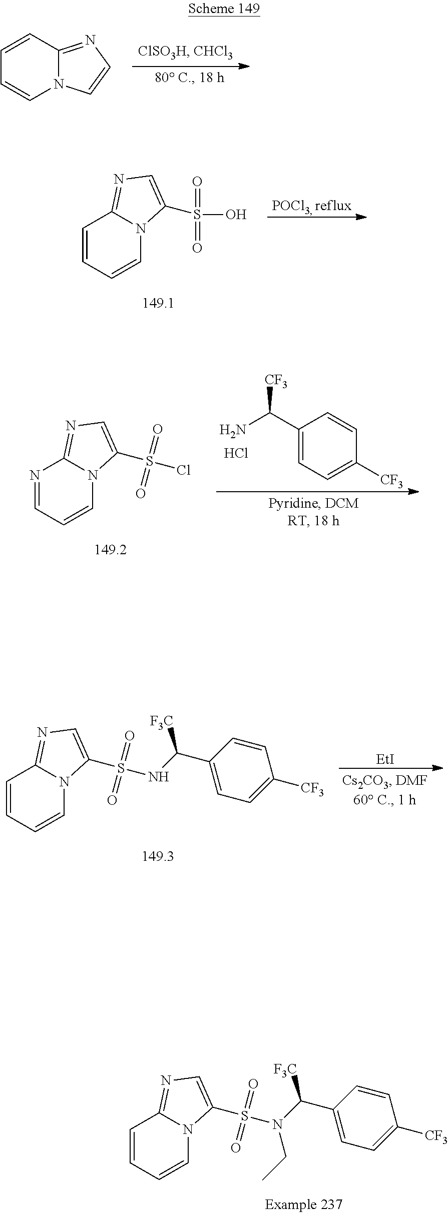

Synthesis of imidazo[1,2-a]pyridine-3-sulfonic acid 149.1: To a stirred suspension of imidazo[1,2-a]pyridine (1.0 g, 8.46 mmol) in CHCl₃ (25 mL) the solution of chlorosulfonic acid (1.7 ml, 25.39 mmol) in CHCl₃ (25 mL) was added dropwise at 0° C. and and the reaction mixture was then refluxed for 18 h. The solution was cooled to room temperature and then concentrated under reduced pressure to afford a pale brown sticky liquid which was solidified with ethanol and diethylether to afford the compound 149.1 as an off-white solid (850 mg, 50% yield). The solid was subjected to forwarding step without further purification.

Synthesis of imidazo[1,2-a]pyridine-3-sulfonyl chloride 149.2: Compound 149.1 (1.60 g, 8.07 mmol) was refluxed with POCl₃ (50 ml) for 18 h. The reaction mass was cooled, concentrated under reduced pressure, and partitioned between DCM (250 mL) and water (150 mL). The organic layer was separated, dried over anhydrous MgSO₄, and concentrated under reduced pressure to afford crude compound (1.70 g) which was used for the next reaction step without further purification.

Synthesis of (R)—N-(2,2,2-trifluoro-1-(4-fluorophenyl)ethyl)imidazo[1,2-a]pyridine-3-sulfonamide 149.3: Intermediate 149.3 was synthesized from sulfonyl chloride 149.2 following the protocol as described in Method AC using (R)-2,2,2-trifluoro-1-(4-(trifluoromethyl)phenyl)ethan-1-amine hydrochloride. Crude compound was purified by column chromatography using 30% ethyl acetate in hexane as eluent to afford desire product 149.3 as off white solid (50 mg, 11% yield). LCMS: m/z found 424.29 (M+H)+, rt=1.86 min (Method A) [Waters Acquity BEH C18 column (1.7 μm, 50×2.1 mm)].

Synthesis of (R)—N-ethyl-N-(2,2,2-trifluoro-1-(4-(trifluoromethyl)phenyl)ethyl)imidazo[1,2-a]pyridine-3-sulfonamide: The ethylation of intermediate 149.3 was performed following the protocol as described in Method AD. The crude was purified by reverse phase chromatography to obtain compound Example 237 as an off-white solid (48 mg, 96.03% purity, 15 yield). Preparative HPLC was done on a WATERS BGM 2545 equipped with WATERS PDA detector 2998 set to multiple-wavelength UV (200-400 nm) detection. Column name: YMC TRIART C18 (250×20 mm, 5μ) operating at ambient temperature and flow rate of 16 mL/min. Mobile phase: A=0.01% Formic Acid in water, B=Acetonitrile. Gradient profile: Mobile phase initial composition of 80% A and 20% B, then to 70% A and 30% B in 3 min, then to 20% A and 80% B in 22 min, then to 0% A and 100% B in 22.5 min, holding in this composition up to 25 min for column washing, then returned to initial composition in 25.5 min and held for 28 min. LCMS: m/z found 452.25 [M+H]⁺, rt=2.42 min (Method 2) [Waters Acquity BEH C18 column (1.7 μm, 50×2.1 mm)]. ¹H NMR (400 MHz, DMSO-d₆): δ 8.87 (d, J=6.8 Hz, 1H), 8.15 (d, J=8.1 Hz, 1H), 8.09 (d, J=9.1 Hz, 1H), 8.05-7.97 (m, 1H), 7.61 (t, J=6.2 Hz, 1H), 7.30 (d, J=8.1 Hz, 2H), 7.22 (d, J=8.2 Hz, 2H), 4.66 (q, J=8.4 Hz, 1H), 4.35-4.11 (m, 2H), 1.26 (t, J=7.2 Hz, 3H).

### Example 238

### (R)—N-methyl-N-(2,2,2-trifluoro-1-(4-(trifluoromethyl)phenyl)ethyl)imidazo[1,2-a]pyridine-7-sulfonamide

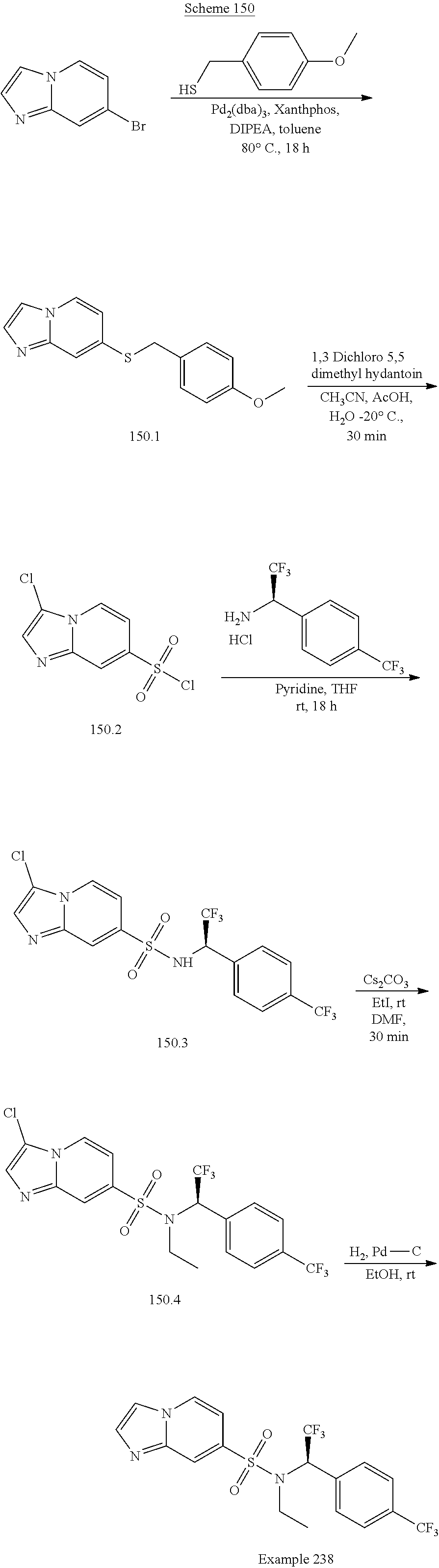

Synthesis of 7-((4-methoxybenzyl)thio)imidazo[1,2-a]pyridine 150.1 Intermediate 150.1 was synthesized following Buchwald protocol as described in Method AA using (4-methoxyphenyl)methanethiol. The crude was purified by column chromatography over silica gel using 50% ethyl acetate in hexane to obtain desired compound 150.1 (72% yield) as a yellow sticky liquid. LCMS: m/z found 271 [M+H+], rt=2.72 min (Method 2) [Waters Acquity BEH C8 column (1.7 μm, 50×2.1 mm)].

Synthesis of 3-chloroimidazo[1,2-a]pyridine-7-sulfonyl chloride 150.2: Intermediate 150.2 was synthesized following the protocol as described in Method T. The crude sulfonyl chloride 150.3 was used for next step sulfonamidation reaction without any purification.

Synthesis of (R)-3-chloro-N-(2,2,2-trifluoro-1-(4-(trifluoromethyl)phenyl)ethyl)imidazo[1,2-a]pyridine-7-sulfonamide 150.3: Intermediate 150.3 was synthesized following the protocol as described in Method AC using (R)-2,2,2-trifluoro-1-(4-(trifluoromethyl)phenyl)ethan-1-amine hydrochloride. The crude was purified by column chromatography over silica gel using 30% ethyl acetate in hexane to obtain desired compound (R)-3-chloro-N-(2,2,2-trifluoro-1-(4-(trifluoromethyl)phenyl)ethyl)imidazo[1,2-a]pyridine-7-sulfonamide 150.3 (300 mg, 52% yield) as a colourless sticky liquid. LCMS: m/z found 458.0 [M+H+], rt=1.72 min (Method 3) [YMC Triart C18 column (3 μm, 33×2.1 mm)].

Synthesis of (R)-3-chloro-N-methyl-N-(2,2,2-trifluoro-1-(4-(trifluoromethyl)phenyl)ethyl)imidazo[1,2-a]pyridine-7-sulfonamide 150.4: The ethylation of intermediate 150.3 was performed following the protocol as described in Method AD. The crude was purified by column chromatography over silica gel using 5:1 ethyl acetate-hexane to obtain compound 150.4 as an off white solid (100 mg, 96.03% purity, 18% yield). LCMS: m/z found 486.20 [M+H]⁺, rt=1.88 min (Method 3) [YMC Triart C18 column (3 μm, 33×2.1 mm)].

Synthesis of (R)—N-methyl-N-(2,2,2-trifluoro-1-(4-(trifluoromethyl)phenyl)ethyl)imidazo[1,2-a]pyridine-7-sulfonamide: The hydrogenation of compound 150.4 was preformed following the protocol as described in Method AE. The crude was purified by normal phase method to obtain pure chiral compound Example 238 as an off-white solid (23.6 mg, 99.23% purity, 35% yield). Method of purification: Chiralpak IG column (30.0 mm×250 mm), 5μ operating at 35° C. temperature, maintaining flow rate of 80 ml/min, using 65% C02 in super critical state & 35% of (100% MeOH) as mobile phase. Run this isocratic mixture up to 10.0 minutes and also maintained the isobaric condition of 100 bar at 235 nm wavelength. LCMS: m/z found 452.35 [M+H]⁺, rt=2.92 min (Method 2) [Waters Acquity BEH C18 column (1.7 μm, 50×2.1 mm)]. ¹H NMR (400 MHz, DMSO-d₆): δ 8.74 (d, J=7.1 Hz, 1H), 8.20 (d, J=7.6 Hz, 2H), 7.85 (s, 1H), 7.77 (d, J=8.2 Hz, 2H), 7.69 (d, J=8.2 Hz, 2H), 7.31 (dd, J=1.4, 7.1 Hz, 1H), 6.25 (q, J=8.7 Hz, 1H), 3.50-3.37 (m, 1H), 3.30-3.14 (m, 1H), 0.97 (t, J=6.9 Hz, 3H).

### Example 239

### (R)—N-ethyl-N-(2,2,2-trifluoro-1-(4-(trifluoromethyl)phenyl)ethyl)imidazo[1,2-a]pyridine-6-sulfonamidesulfonamide

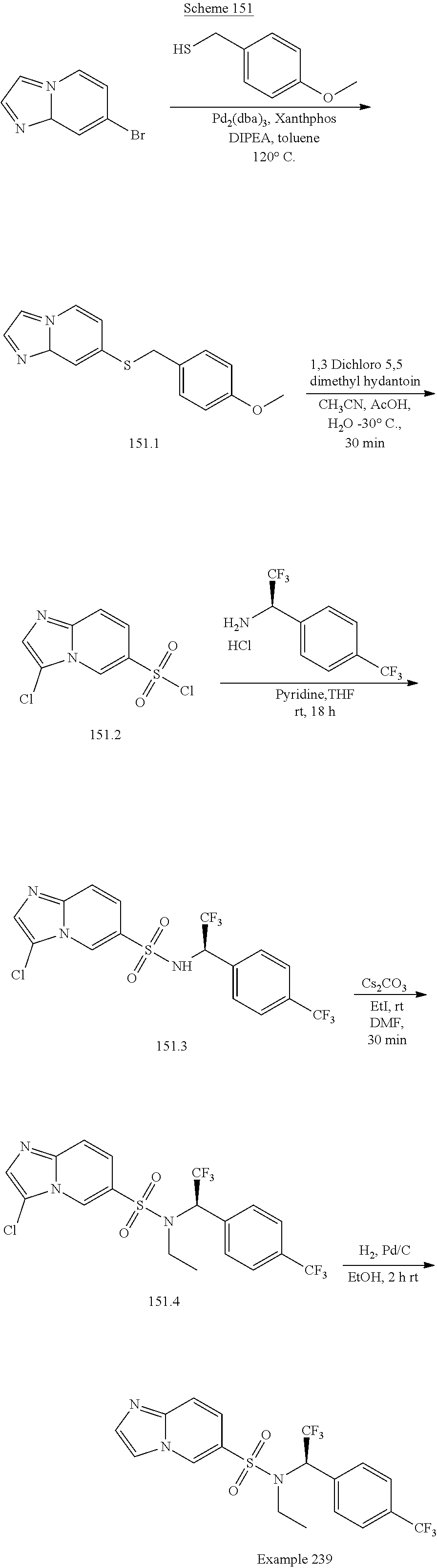

Synthesis of 6-((4-methoxybenzyl)thio)imidazo[1,2-a]pyridine 151.1 Intermediate 151.1 was synthesized following Buchwald protocol as described in Method AA using (4-methoxyphenyl)methanethiol. The crude was purified by column chromatography over silica gel using 50% ethyl acetate in hexane to obtain the desired compound 151.1 (80% yield) as a yellow sticky gum. LCMS: m/z found 271 [M+H+], rt=2.72 min (Method 2) [Waters Acquity BEH C8 column (1.7 μm, 50×2.1 mm)].

Synthesis of 3-chloroimidazo[1,2-a]pyridine-6-sulfonyl chloride 151.2: Intermediate 151.2 was synthesized following the protocol as described in Method AB. The crude sulfonyl chloride 151.2 was used for forwarding step without any purification.

Synthesis of (R)-3-chloro-N-(2,2,2-trifluoro-1-(4-(trifluoromethyl)phenyl)ethyl)imidazo[1,2-a]pyridine-6-sulfonamide 151.3: Intermediate 151.3 was synthesized following the protocol as described in Method AC using (R)-2,2,2-trifluoro-1-(4-(trifluoromethyl)phenyl)ethan-1-amine hydrochloride. The crude was purified by column chromatography over silica gel using 30% ethyl acetate in hexane to obtain desired compound 151.3 (150 mg, 12% yield) as a colorless sticky liquid. LCMS: m/z found 458.0[M+H+], rt=1.72 min (Method 3) [YMC Triart C18 column (3 μm, 33×2.1 mm)].

Synthesis of (R)-3-chloro-N-methyl-N-(2,2,2-trifluoro-1-(4-(trifluoromethyl)phenyl)ethyl)imidazo[1,2-a]pyridine-7-sulfonamide 151.4: The ethylation of intermediate 151.3 was performed following the protocol as described in Method AD. The crude was purified by column chromatography over silica gel using 1:5 ethyl acetate-hexane to obtain compound 151.4 as an off-white solid (100 mg, 12% yield). LCMS: m/z found 486.19 [M+H]⁺, rt=3.17 min (Method 2) [Waters Acquity BEH C8 column (1.7 μm, 50×2.1 mm)].

Synthesis of (R)—N-ethyl-N-(2,2,2-trifluoro-1-(4-(trifluoromethyl)phenyl)ethyl)imidazo[1,2-a]pyridine-6-sulfonamidesulfonamide: The hydrogenation of compound 151.4 was preformed following the protocol as described in Method AE. The crude was purified by normal phase method to obtain pure chiral compound Example 239 as an off-white solid (5 mg, 99.23% purity, 15% yield). Method of purification: CHIRALPAK-IG Column (30.0 mm×250 mm), 5μ operating at 35° C. temperature, maintaining flow rate of 60 mL/min, using 70% CO₂ in super critical state and 30% of MeOH as mobile phase. Run this isocratic mixture up to 20 minutes and also maintained the isobaric condition of 100 bar at 220 nm wavelength. LCMS: m/z found 452.35 [M+H]⁺, rt=rt 2.87 min (Method 2) [Waters Acquity BEH C18 column (1.7 μm, 50×2.1 mm)]. ¹H NMR (400 MHz, Chloroform-d): δ 8.88 (s, 1H), 8.13-7.93 (m, 1H), 7.88 (s, 1H), 7.79 (s, 1H), 7.74-7.60 (m, 4H), 5.90 (q, J=8.2 Hz, 1H), 3.50-3.36 (m, 1H), 3.25-3.11 (m, 1H), 1.37-1.17 (m, 1H), 0.94 (t, J=7.1 Hz, 3H).

### Example 240

### (R)-5-cyano-N-(1-(4-(difluoromethoxy)phenyl)-2,2,2-trifluoroethyl)-N-methylpyridine-3-sulfonamide

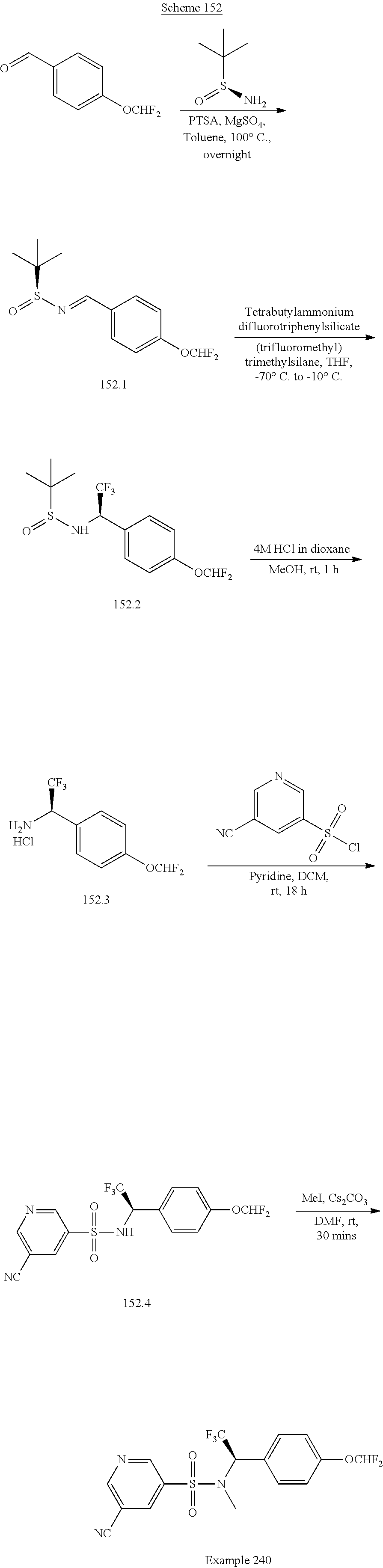

Synthesis of (S)—N-(4-(difluoromethoxy)benzylidene)-2-methylpropane-2-sulfinamide 152.1: Method P-I: To a stirred solution of 4-(difluoromethoxy)benzaldehyde (2.0 g, 11.61 mmol) and 2-methylpropane-2-sulfinamide (1.69 g, 13.94 mmol) in toluene (20 mL) PTSA (100 mg) was added, followed by anhydrous MgSO₄. The reaction mixture was stirred at 80° C. for 16 h. It was cooled to room temperature and the reaction mixture was filtered through celite. The volatiles were evaporated under reduced pressure and the crude was purified by column chromatography over silica gel using 10% ethyl acetate in hexane to yield the desired compound as a pale-yellow solid (1.20 g, 37% yield). LCMS: m/z found 276.0[M+H]⁺, rt=1.75 min (Method 3) [YMC Triart C18 column (3 μm, 33×2.1 mm)].

Synthesis of N—((R)-1-(4-(difluoromethoxy)phenyl)-2,2,2-trifluoroethyl)-2-methylpropane-2-sulfinamide 152.2: Method Q-I: To a solution of intermediate 152.1 (1.0 g, 0.88 mmol) in THF (2 mL) tetrabutylammonium difluorotriphenylsilicate (95 mg, 0.17 mmol) in THF (1 mL) was added at −70° C. and the reaction mixture was stirred for 30 min. CF₃TMS (0.4 mL, 2.64 mmol) was then added dropwise at the same temperature and the reaction mixture was stirred for 4 h at −70° C. It was then warmed to ambient temperature and then allowed to stir for 18 h. The reaction mixture was quenched at −10° C. with aq. std. NH₄Cl solution (2 mL). It was then diluted with aq. std. NH₄Cl solution (10 mL) and extracted with ethyl acetate (2×20 mL). Combined organic layer was washed with water (10 mL) and brine (10 mL), dried and the solvent was evaporated under reduced pressure. The crude was purified by column chromatography over silica gel and the compound 152.2 was isolated as a colorless gum (600 mg, yield 47%). LCMS: m/z found 346.12 [M+H]⁺, rt=2.12 min (Method 32) [Waters Acquity BEH C18 column (1.7 μm, 50×2.1 mm)].

Synthesis of (R)-1-(4-(difluoromethoxy)phenyl)-2,2,2-trifluoroethan-1-amine hydrochloride 152.3: Intermediate 152.2 (200 mg) was dissolved in MeOH (2 ml) and cooled. 4 M HCl in dioxane (1 mL) was added to it and the reaction mixture was stirred at room temperature for 1 h. The volatiles were evaporated under reduced pressure and the crude compound 1.3 was used in the forwarding step without further purification.

Synthesis of (R)-5-cyano-N-(1-(4-(difluoromethoxy)phenyl)-2,2,2-trifluoroethyl)pyridine-3-sulfonamide 152.4: Intermediate 152.4 was synthesized from 5-cyanopyridine-3-sulfonyl chloride following the protocol as described in Method O using (R)-1-(4-(difluoromethoxy)phenyl)-2,2,2-trifluoroethan-1-amine hydrochloride in DCM. The compound was purified by column chromatography over silica gel using 25% ethyl acetate in hexane and isolated as a colorless gum (600 mg, 64% yield). LCMS: m/z found 408.12 [M+H]⁺, rt=2.10 min (Method 32) [Waters Acquity BEH C18 column Waters Acquity BEH C18 column (1.7 μm, 50×2.1 mm)].

Synthesis of (R)-5-cyano-N-(1-(4-(difluoromethoxy)phenyl)-2,2,2-trifluoroethyl)-N-methylpyridine-3-sulfonamide: The methylation of intermediate 15.3 was preformed following the protocol as described in Method D using Cs₂CO₃. The crude was purified by combiflash chromatography to obtain compound Example 240 as an off-white solid (65 mg, 99.57% purity, 48% yield). LCMS: m/z found 422.31 [M+H]⁺, rt=3.04 min (Method 2) [Waters Acquity BEH C18 column (1.7 μm, 50×2.1 mm)]. ¹H NMR (400 MHz, DMSO-d₆): δ 8.87 (d, J=6.8 Hz, 1H), 8.15 (d, J=8.1 Hz, 1H), 8.09 (d, J=9.1 Hz, 1H), 8.05-7.97 (m, 1H), 7.61 (t, J=6.2 Hz, 1H), 7.30 (d, J=8.1 Hz, 2H), 7.22 (d, J=8.2 Hz, 2H), 4.66 (q, J=8.4 Hz, 1H), 4.35-4.11 (m, 2H), 1.26 (t, J=7.2 Hz, 3H).

### Example 241

### (R)-5-cyano-N-methyl-N-(2,2,2-trifluoro-1-(4-(trifluoromethoxy)phenyl)ethyl)pyridine-3-sulfonamide

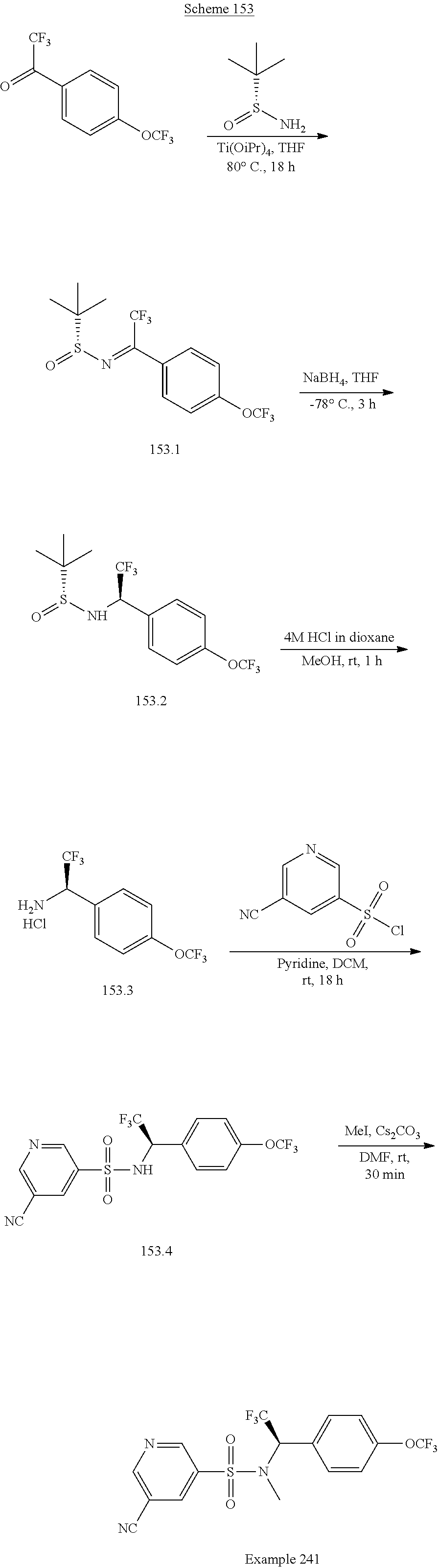

Synthesis of (R)-2-methyl-N-(2,2,2-trifluoro-1-(4-(trifluoromethoxy)phenyl)ethylidene)propane-2-sulfinamide 153.1: Intermediate 153.1 was synthesized from 2,2,2-trifluoro-1-(4-(trifluoromethoxy)phenyl)ethan-1-one following the protocol as described in Method P-I. The reaction mass was used in the forwarding step without any work up.

Synthesis of (R)-2-methyl-N—((R)-2,2,2-trifluoro-1-(4-(trifluoromethoxy)phenyl)ethyl)propane-2-sulfinamide 153.2: Intermediate 153.2 was synthesized from intermediate 153.1 following the protocol as described in Method Y. The compound was purified by column chromatography over silica gel using 45% ethyl acetate in hexane and isolated as colourless gum (1.4 g, 48% yield). LCMS: m/z found 364.3 [M+H]⁺, rt=2.01 min (Method 2) [Waters Acquity BEH C18 column (1.7 μm, 50×2.1 mm)].

Synthesis of (R)-2,2,2-trifluoro-1-(4-(trifluoromethoxy)phenyl)ethan-1-amine hydrochloride 153.3: Intermediate 152.2 (1.4 g) was dissolved in MeOH (10 ml) under cooling. 4 M HCl in dioxane (10 mL) was added to it and the reaction mixture was stirred at room temperature for 1 h. The volatiles were evaporated under reduced pressure and the crude compound 153.3 was used in the forwarding step without further purification.

Synthesis of (R)-5-cyano-N-(2,2,2-trifluoro-1-(4-(trifluoromethoxy)phenyl)ethyl)pyridine-3-sulfonamide 153.4: Intermediate 153.4 was synthesized from 5-cyanopyridine-3-sulfonyl chloride following the protocol as described in Method O using (R)-2,2,2-trifluoro-1-(4-(trifluoromethoxy)phenyl)ethan-1-amine hydrochloride in DCM. The compound was purified by column chromatography over silica gel using 25% ethyl acetate in hexane and isolated as colourless gum (300 mg, 54% yield). LCMS: m/z found 425.9 [M+H]⁺, rt=1.75 min (Method 3) [YMC Triart C18 column (3 μm, 33×2.1 mm)].

Synthesis of (R)-5-cyano-N-methyl-N-(2,2,2-trifluoro-1-(4-(trifluoromethoxy)phenyl)ethyl)pyridine-3-sulfonamide: The methylation of intermediate 153.4 was performed following the protocol as described in Method D using Cs2CO3. The crude was purified by combiflash chromatography to obtain compound Example 241 as an off-white solid (55 mg, 96.03% purity, 43% yield). LCMS: m/z found 440.30 [M+H]⁺, rt=3.15 min (Method 2) [Waters Acquity BEH C18 column (1.7 μm, 50×2.1 mm)]. ¹H NMR (400 MHz, DMSO-d₆): δ 8.87 (d, J=6.8 Hz, 1H), 8.15 (d, J=8.1 Hz, 1H), 8.09 (d, J=9.1 Hz, 1H), 8.05-7.97 (m, 1H), 7.61 (t, J=6.2 Hz, 1H), 7.30 (d, J=8.1 Hz, 2H), 7.22 (d, J=8.2 Hz, 2H), 4.66 (q, J=8.4 Hz, 1H), 4.35-4.11 (m, 2H), 1.26 (t, J=7.2 Hz, 3H).

### Example 242

### N-(1-(4-chlorophenyl)-2,2,2-trifluoroethyl-1-d)-5-cyano-N-methylpyridine-3-sulfonamide

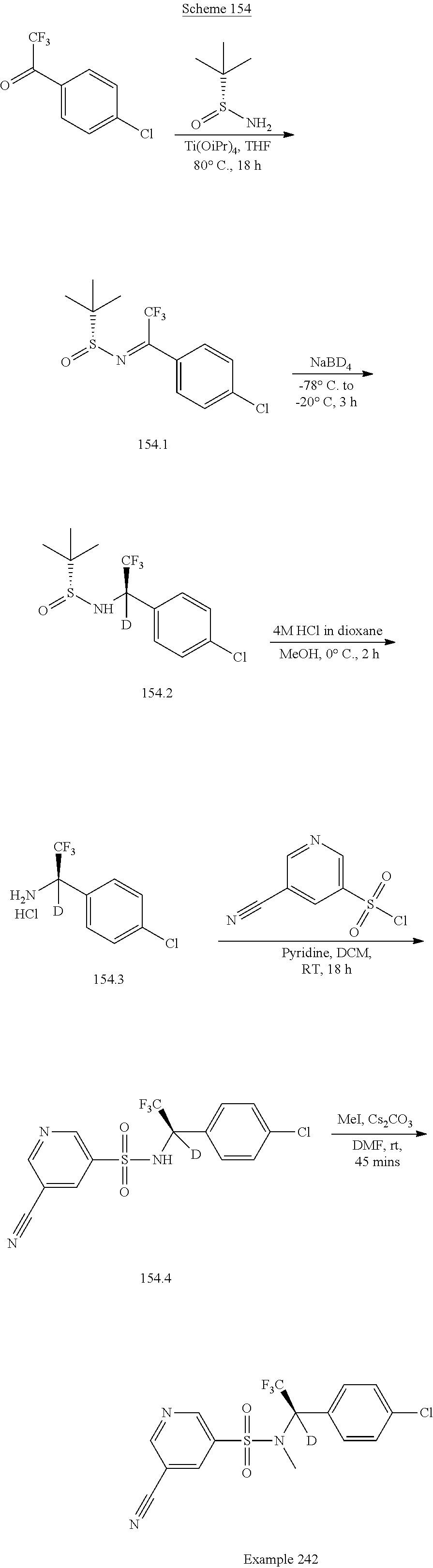

Synthesis of (R)—N-(1-(4-chlorophenyl)-2,2,2-trifluoroethylidene)-2-methylpropane-2-sulfinamide 154.1: Imine formation; Method P-I: To a stirred solution of (R)-2-methylpropane-2-sulfinamide (0.72 g, 5.99 mmol) in THF (5 mL) Ti(OiPr)₄ (3.83 mL, 12.94 mmol) was added and the reaction mixture was stirred for 15 min at room temperature. 1-(4-chlorophenyl)-2,2,2-trifluoroethan-1-one (1.0 g, 4.79 mmol) was added to it and the reaction mixture was heated at 80° C. for 18 h. The crude reaction mixture was forwarded to forwarding reduction step without any work up and purification.

Synthesis of (R)—N-(1-(4-chlorophenyl)-2,2,2-trifluoroethyl-1-d)-2-methylpropane-2-sulfinamide 154.2: Reduction; Method Y: The reaction mixture of previous step was cooled at −78° C. and NaBD₄ (0.403 g, 9.62 mmol) was added portion wise maintaining the same temperature. It was then warmed to −20° C. and stirred for 3 h. The reaction mixture was poured gradually on an ice cold saturated NaCl solution with vigorous stirring. The resulting suspension was extracted with ethyl acetate (4×20 mL) and the organic part was then filtered through a plug of celite. The filtrate was further washed with saturated NaCl solution (20 mL) and dried (anhydrous Na₂SO₄). The volatiles were evaporated under reduced pressure and the crude product was purified by column chromatography over silica gel using 30% ethyl acetate in hexane to obtain the intermediate 154.2 as a light-yellow gum (910 mg, 90% yield). LCMS: m/z found 315.2 [M+H]⁺, rt=1.82 min (Method 8) [Waters Xbridge C18 column (3.5 μm, 50×3 mm)]. ¹H NMR (400 MHz, DMSO): δ 7.59 (d, J=8.1 Hz, 2H), 7.48 (d, J=8.1 Hz, 2H), 6.70 (s, 1H), 1.04 (s, 9H).

Synthesis of 1-(4-chlorophenyl)-2,2,2-trifluoroethan-1-d-1-amine hydrochloride 154.3: Intermediate 154.2 (900 mg, 2.85 mmol) was dissolved in MeOH (5 mL) and cooled at 0° C. 4 M HCl in dioxane (10 mL) was added to it and the RM was stirred at room temperature for 2 h. The volatiles were evaporated under reduced pressure and the crude compound 154.3 triturated with 50% diethyl ether in pentane. The desired compound 154.3 was isolated as hydrochloride salt (700 mg) and was used in the forwarding step without further purification. LCMS: m/z found 211.1 [M+H], rt=1.97 min (Method 8) [Waters Xbridge C18 column (3.5 μm, 50×3 mm)]. ¹H NMR (400 MHz, DMSO): δ 10.04-9.41 (m, 3H), 7.72-7.59 (m, 4H).

Synthesis of N-(1-(4-chlorophenyl)-2,2,2-trifluoroethyl-1-d)-5-cyanopyridine-3-sulfonamide 154.4: Method O: 5-cyanopyridine-3-sulfonyl chloride (200 mg, 0.98 mmol) was dissolved in DCM (3.0 mL) and 1-(4-chlorophenyl)-2,2,2-trifluoroethan-1-d-1-amine hydrochloride (250 mg, 0.98 mmol) was added to it followed by pyridine (0.8 mL, 9.87 mmol). The reaction mixture was stirred at room temperature overnight. It was diluted with water (10 mL) and extracted with DCM (2×20 mL). The combined organic layers were washed with water (20 mL) and brine (10 mL), dried and the solvent was evaporated under reduced pressure. The crude was purified by column chromatography over silica gel and the compound 154.4 was isolated as off white solid (190 mg, 51% yield). LCMS: m/z found 376.9 [M+H]⁺, rt=1.69 min (Method 3) [YMC Triart C18 column (3 μm, 33×2.1 mm)]. ¹H NMR (400 MHz, DMSO): δ 9.85 (s, 1H), 9.16 (d, J=1.4 Hz, 1H), 9.02 (d, J=2.0 Hz, 1H), 8.45 (t, J=2.1 Hz, 1H), 7.49-7.32 (m, 4H).

Synthesis of N-(1-(4-chlorophenyl)-2,2,2-trifluoroethyl-1-d)-5-cyano-N-methylpyridine-3-sulfonamide: Alkylation; Method D: The sulfonamide 154.4 (0.180 g, 0.478 mmol) was dissolved in DMF (1 mL) in a sealed tube and Cs₂CO₃ (233 mg, 0.72 mmol) was added to it. It was stirred and to it was added methyl iodide (0.15 mL, 2.38 mmol). The reaction mixture was then stirred at room temperature for 45 min. The reaction was then quenched with water (10 mL) and extracted with ethyl acetate (2×10 mL). The combined organic layer was washed with water (2×10 mL) and brine (2×10 mL), dried over anhydrous Na₂SO₄ and concentrated under reduced pressure. The crude was purified by column chromatography over silica gel using 20% ethyl acetate in hexane and the product Example 242 was isolated as a colorless sticky gum (140 mg, 75% yield). LCMS: m/z found 391.17 [M+H]⁺, rt=3.06 min (Method 2) [Waters Acquity BEH C8 column (1.7 μm, 50×2.1 mm)]. ¹H NMR (400 MHz, DMSO): δ 9.85 (s, 1H), 9.16 (d, J=1.4 Hz, 1H), 9.02 (d, J=2.0 Hz, 1H), 8.45 (t, J=2.1 Hz, 1H), 7.49-7.32 (m, 4H).

### Example 243 and Example 244

### (S)-5-cyano-N-(1-(2,4-difluorophenyl)-2,2,2-trifluoroethyl)-N-ethylpyridine-3-sulfonamide (Example 243) and (R)-5-cyano-N-(1-(2,4-difluorophenyl)-2,2,2-trifluoroethyl)-N-ethylpyridine-3-sulfonamide (Example 244)

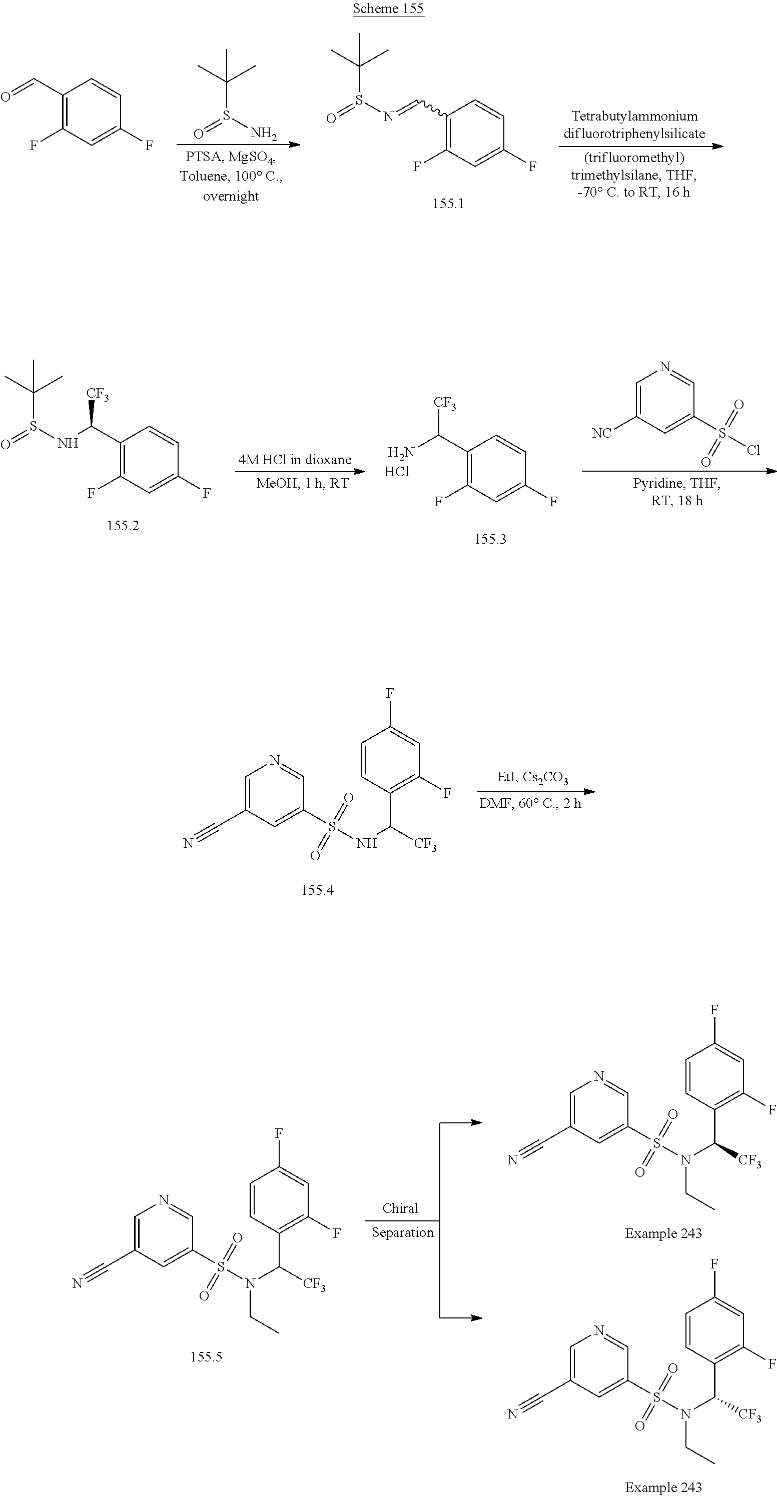

Synthesis of N-(2,4-difluorobenzylidene)-2-methylpropane-2-sulfinamide 155.1: Method P-II: To a stirred solution of 2 2,4-difluorobenzaldehyde (2.0 g, 14.07 mmol) and 2-methylpropane-2-sulfinamide (4.14 g, 16.88 mmol) in toluene (20 mL PTSA (100 mg)) was added followed by anhydrous MgSO4. The reaction mixture was stirred at 80° C. for 16 h. It was cooled to room temperature and the reaction mixture was filtered through celite. The volatiles were evaporated under reduced pressure and the crude was purified by column chromatography over silica gel using 10% EA in hexane to yield the desired compound as yellow oil (400 mg, 34% yield). ¹H NMR (400 MHz, DMSO-d₆): δ 8.64 (s, 1H), 8.12-8.05 (m, 1H), 7.52-7.45 (m, 1H), 7.31-7.25 (m, 1H), 1.18 (s, 9H).

Synthesis of N-(1-(2,4-difluorophenyl)-2,2,2-trifluoroethyl)-2-methylpropane-2-sulfinamide 155.2: Method Q-I: To a solution of intermediate 155.1 (1,0 g, 0.88 mmol) in THF (2 mL) tetrabutylammonium difluorotriphenylsilicate (95 mg, 0.17 mmol) in THF (1 mL) was added at −70° C. and the reaction mixture was stirred for 30 min. CF₃TMS (0.4 mL, 2.64 mmol) was added dropwise at the same temperature and the reaction mixture was stirred for 4 h at −70° C. It was then warmed to −10° C. and stirred for another 4 h. The reaction mixture was quenched at −10° C. with aq. std. NH₄Cl solution (2 mL). It was then diluted with aq. std. NH₄Cl solution (10 mL) and extracted with ethyl acetate (2×20 mL). Combined organic layers were washed with water (10 mL) and brine (10 mL), dried, and the solvent was evaporated under reduced pressure. The crude was purified by CC over silica gel and the compound 155.2 was isolated as a colourless gum (200 mg, yield 75%). ¹H NMR (400 MHz, DMSO-d₆): δ 7.84-7.80 (m, 1H), 7.41-7.32 (m, 1H), 7.30-7.22 (m, 1H), 6.67 (d, J=9.5 Hz, 1H), 5.30-5.23 (m, 1H), 1.10 (s, 9H).

Synthesis of 1-(2,4-difluorophenyl)-2,2,2-trifluoroethan-1-amine hydrochloride 155.3: Intermediate 155.2 (200 mg) was dissolved in MeOH (2 ml) and cooled. 4 M HCl in dioxane (1 mL) was added to it and the reaction mixture was stirred at room temperature for 1 h. The volatiles were evaporated under reduced pressure and the crude compound 155.3 was used in the forwarding step without further purification.

Synthesis of 5-cyano-N-(1-(2,4-difluorophenyl)-2,2,2-trifluoroethyl)pyridine-3-sulfonamide 155.4: Intermediate 155.4 was synthesized from 5-cyanopyridine-3-sulfonyl chloride following the protocol as described in Method O using 1-(2,4-difluorophenyl)-2,2,2-trifluoroethan-1-amine hydrochloride. The compound was purified by column chromatography over silica gel using 25% ethyl acetate in hexane and isolated as colourless gum (600 mg, 64% yield). ¹H NMR (400 MHz, DMSO-d₆): δ 10.02 (s, 1H), 9.20 (d, J=1.4 Hz, 1H), 9.04 (d, J=1.9 Hz, 1H), 7.60-7.46 (m, 1H), 7.35-7.25 (m, 1H), 7.11 (t, J=6.3 Hz, 1H), 5.55-5.35 (m, 1H).

Synthesis of 5-cyano-N-(1-(2,4-difluorophenyl)-2,2,2-trifluoroethyl)-N-ethylpyridine-3-sulfonamide 155.5: The ethylation of intermediate 155.4 was preformed following the protocol as described in Method D at 60° C. using Cs₂CO₃. The crude was purified by column chromatography over silica gel using 20% ethyl acetate in hexane and the desired compound 155.5 was isolated as light-yellow sticky gum (150 mg, 30% yield). LCMS: m/z found 406.3 [M+H]⁺, rt=3.38 min (Method 2) [Waters Acquity BEH C18 column (1.7 μm, 50×2.1 mm)]. Chiral separation of racemic compound 155.5 provide both the enantiomers. Chiral separation method: Chiral separation method: Chiral separation was done on Agilent 1200 series instrument. Column name: Chiralpak IG (4.6×250 mm), 5p. Operating at ambient temperature and flow rate is 1.0 ml/min. Mobile phase was mixture of 70% Hexane, 30% EtOH and 0.1% IPAMINE, held this isocratic mixture run up to 15 min with wavelength of 246 nm.

Example 243: Light yellow sticky gum (45 mg, 97.71% purity). LCMS: m/z found 406.3 [M+H]⁺, rt=3.38 min (Method 2) [Waters Acquity BEH C18 column (1.7 μm, 50×2.1 mm)]; ¹H NMR (400 MHz, Chloroform-d): δ 9.20 (d, J=2.0 Hz, 1H), 9.02 (d, J=1.8 Hz, 1H), 8.35 (t, J=2.0 Hz, 1H), 7.55 (q, J=8.2 Hz, 1H), 7.03-6.87 (m, 2H), 6.07 (q, J=8.2 Hz, 1H), 3.32 (q, J=7.1 Hz, 2H), 0.92 (t, J=7.0 Hz, 3H).

Example 244: Light yellow sticky gum (28 mg, 99.12% purity). LCMS: m/z found 406.3 [M+H]⁺, rt=3.38 min (Method 2) [Waters Acquity BEH C18 column (1.7 μm, 50×2.1 mm)]; ¹H NMR (400 MHz, Chloroform-d) δ 9.20 (d, J=2.0 Hz, 1H), 9.02 (d, J=1.8 Hz, 1H), 8.35 (t, J=2.0 Hz, 1H), 7.55 (q, J=8.1 Hz, 1H), 7.03-6.87 (m, 2H), 6.06 (q, J=8.3 Hz, 1H), 3.32 (q, J=7.1 Hz, 2H), 0.92 (t, J=7.0 Hz, 3H).

### Example 245 and Example 246

### (S)-5-cyano-N-ethyl-N-(2,2,2-trifluoro-1-(4-fluoro-3-methylphenyl)ethyl)pyridine-3-sulfonamide (Example 245) and (R)-5-cyano-N-ethyl-N-(2,2,2-trifluoro-1-(4-fluoro-3-methylphenyl)ethyl)pyridine-3-sulfonamide (Example 246)

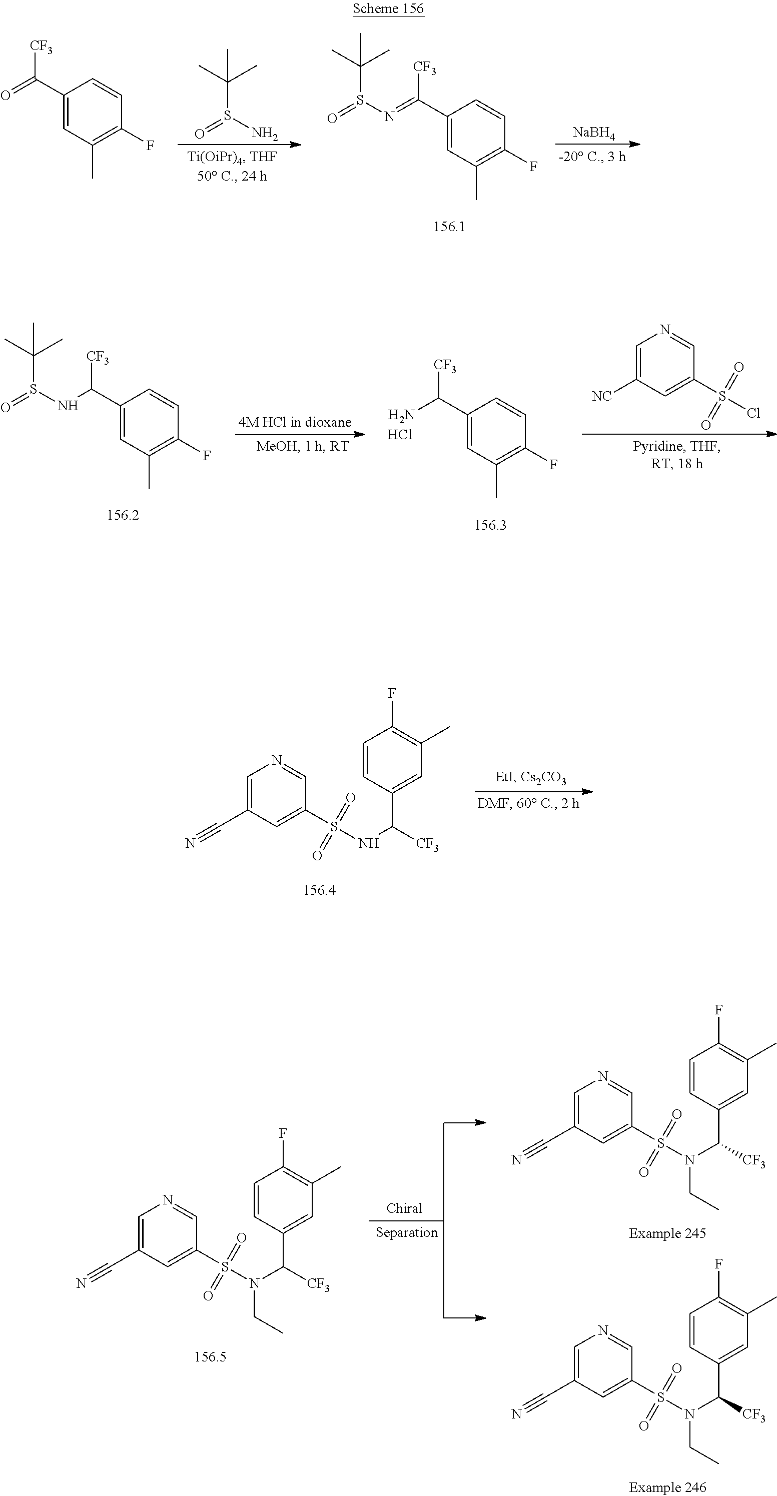

Synthesis of 2-methyl-N-(2,2,2-trifluoro-1-(4-fluoro-3-methylphenyl)ethylidene)propane-2-sulfinamide 156.1: Intermediate 156.1 was synthesized from 2,2,2-trifluoro-1-(4-fluoro-3-methylphenyl)ethan-1-one following the protocol as described in Method P-I. The reaction mass was used in the forwarding step without any work up.

Synthesis of 2-methyl-N-(2,2,2-trifluoro-1-(4-fluoro-3-methylphenyl)ethyl)propane-2-sulfinamide 156.2: Intermediate 156.2 was synthesized from intermediate 156.1 following the protocol as described in Method Y. The compound was purified by column chromatography over silica gel using 45% ethyl acetate in hexane and isolated as a colourless gum (1.40 g, 48% yield). ¹H NMR (400 MHz, Chloroform-d): δ 7.25-7.20 (m, 2H), 7.05-7.00 (m, 1H), 4.86-4.75 (m, 1H), 3.85-3.80 (m, 1H), 2.28 (s, 3H), 1.24 (s, 9H).

Synthesis of 2,2,2-trifluoro-1-(4-fluoro-3-methylphenyl)ethan-1-amine 156.3: Intermediate 156.2 (1.40 g) was dissolved in MeOH (10 ml) and cooled. 4 M HCl in dioxane (10 mL) was added to it and the reaction mixture was stirred at room temperature for 1 h. The volatiles were evaporated under reduced pressure and the crude compound 156.3 was used in the forwarding step without further purification.

Synthesis of 5-cyano-N-(2,2,2-trifluoro-1-(4-fluoro-3-methylphenyl)ethyl)pyridine-3-sulfonamide 156.4: Intermediate 156.4 was synthesized from 5-cyanopyridine-3-sulfonyl chloride following the protocol as described in Method O using 2,2,2-trifluoro-1-(4-fluoro-3-methylphenyl)ethan-1-amine hydrochloride. The compound was purified by column chromatography over silica gel using 25% ethyl acetate in hexane and isolated as colourless gum (650 mg, 80% yield). ¹H NMR (400 MHz, DMSO-d₆): δ 9.75-9.65 (m, 1H), 9.13 (s, 1H), 8.98 (s, 1H), 8.39 (s, 1H), 7.38-7.27 (m, 1H), 7.27-7.19 (m, 1H), 7.02 (t, J=8.4 Hz, 2H), 5.4-5.3 (m, 1H), 2.12 (s, 3H).

Synthesis of 5-cyano-N-ethyl-N-(2,2,2-trifluoro-1-(4-fluoro-3-methylphenyl)ethyl)pyridine-3-sulfonamide 156.5: The ethylation of intermediate 156.5 was preformed following the protocol as described in Method D. The crude was purified by column chromatography over silica gel using 1:5 ethyl acetate-hexane to obtain compound 156.5 as colorless gum (350 mg, 49% yield). LCMS: m/z found 402.3 [M+H]⁺, rt=3.50 min (Method 2) [Waters Acquity BEH C18 column (1.7 μm, 50×2.1 mm)]. Chiral separation of racemic compound 156.5 provide both the enantiomers. Chiral separation method: Chiral separation was done on Agilent 1200 series instrument. Column name: Chiralpak IG (4.6×250 mm), 5p. Operating at ambient temperature and flow rate is 1.0 ml/min. Mobile phase was mixture of 70% Hexane, 30% EtOH and 0.1% IPAMINE, held this isocratic mixture run up to 15 min with wavelength of 246 nm.

Example 245: Light yellow sticky gum (70 mg, 99.22% purity). LCMS: m/z found 402.3 [M+H]⁺, rt=3.50 min (Method 2) [Waters Acquity BEH C18 column (1.7 μm, 50×2.1 mm)]; ¹H NMR (400 MHz, Chloroform-d): δ 9.21 (d, J=2.1 Hz, 1H), 9.02 (d, J=1.9 Hz, 1H), 8.35 (t, J=2.0 Hz, 1H), 7.27 (d, J=6.4 Hz, 2H), 7.10-6.98 (m, 1H), 5.72 (q, J=8.4 Hz, 1H), 3.46-3.30 (m, 1H), 3.29-3.13 (m, 1H), 2.28 (s, 3H), 0.93 (t, J=7.1 Hz, 3H).

Example 246: Light yellow sticky gum (52 mg, 95.11% purity). LCMS: m/z found 402.3 [M+H]⁺, rt=3.50 min (Method 2) [Waters Acquity BEH C18 column (1.7 μm, 50×2.1 mm)]; ¹H NMR (400 MHz, Chloroform-d): δ 9.23 (s, 1H), 9.04 (s, 1H), 8.36 (s, 1H), 7.29 (d, J=6.2 Hz, 2H), 7.05 (t, J=9.2 Hz, 1H), 5.73 (q, J=8.4 Hz, 1H), 3.45-3.32 (m, 1H), 3.31-3.17 (m, 1H), 2.30 (s, 3H), 0.95 (d, J=6.8 Hz, 3H).

### Example 247 and Example 248

### (S)-5-cyano-N-ethyl-N-(2,2,2-trifluoro-1-(2-(trifluoromethyl)phenyl)ethyl)pyridine-3-sulfonamide (Example 247) and (R)-5-cyano-N-ethyl-N-(2,2,2-trifluoro-1-(2-(trifluoromethyl)phenyl)ethyl)pyridine-3-sulfonamide (Example 248)

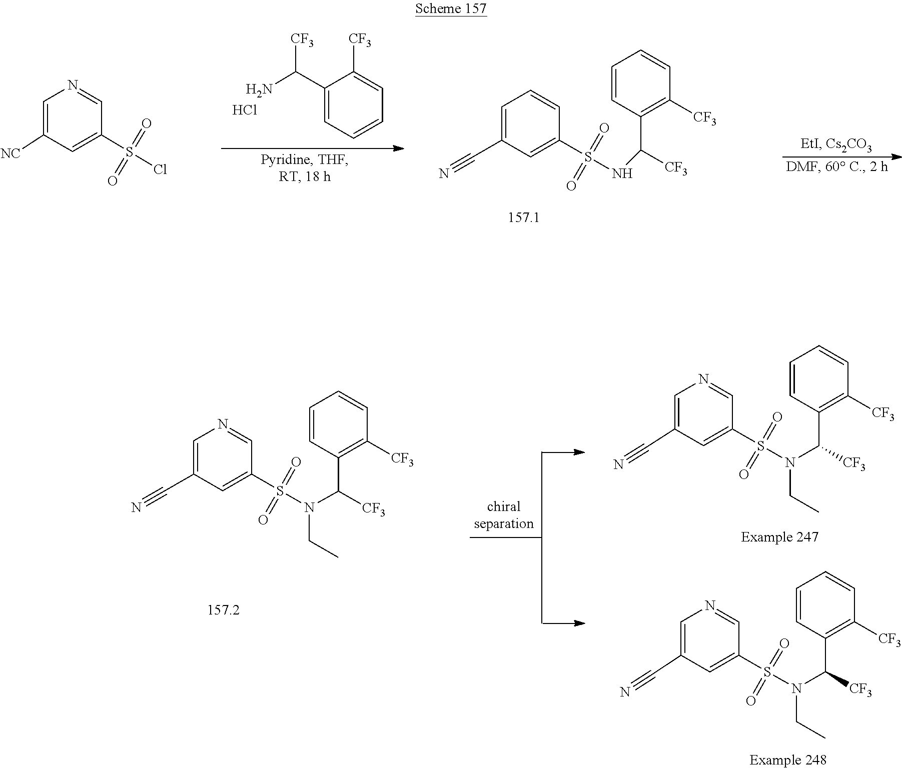

Synthesis of 5-cyano-N-(2,2,2-trifluoro-1-(2-(trifluoromethyl)phenyl)ethyl)pyridine-3-sulfonamide 157.1: Sulfonamide 157.1 was synthesized from 5-cyanopyridine-3-sulfonyl chloride following the protocol as described in Method O using 2,2,2-trifluoro-1-(2-(trifluoromethyl)phenyl)ethan-1-amine hydrochloride. The compound was purified by column chromatography over silica gel using 40% ethyl acetate in hexane to isolate desired compound 157.1 as yellow gum (400 mg, 60% yield). LCMS: m/z found 410.0 [M+H]⁺, rt=1.84 min (Method 3) [YMC Triart C18 column (3 μm, 33×2.1 mm)].

Synthesis of 5-cyano-N-ethyl-N-(2,2,2-trifluoro-1-(2-(trifluoromethyl)phenyl)ethyl)pyridine-3-sulfonamide 157.2: The ethylation of intermediate 157.1 was preformed following the protocol as described in Method D. The crude was purified by column chromatography over silica gel using 20% ethyl acetate-hexane to obtain compound 157.2 as colorless gum (100 mg, 30% yield). Chiral separation of racemic compound 157.2 provide both the enantiomers. Chiral separation was done on an Agilent 1200 series instrument. Column name: Chiralcel OJ-H (4.6×250 mm), 5p. Operating at ambient temperature and flow rate is 1.0 mL/min. Mobile phase was the mixture of 60% Hexane and 20% EtOH and 0.1 mL isopropylamine, held this isocratic mixture run up to 25 min with wavelength of 264 nm.

Example 247: Brown sticky gum (52 mg, 98.95% purity). LCMS: m/z found 438.19 [M+H]⁺, rt=2.56 min (Method B) [Waters Acquity BEH C8 column (1.7 μm, 50×2.1 mm)]; ¹H NMR (400 MHz, Chloroform-d): δ 9.20 (d, J=2.1 Hz, 1H), 9.02 (d, J=1.8 Hz, 1H), 8.32 (t, J=2.2 Hz, 1H), 7.85 (dd, J=2.1, 7.2 Hz, 1H), 7.78 (d, J=7.2 Hz, 1H), 7.72-7.60 (m, 2H), 6.30 (q, J=8.0 Hz, 1H), 3.40 (q, J=7.0 Hz, 2H), 0.67 (t, J=7.0 Hz, 3H).

Example 248: Brown sticky gum (40 mg, 96.84% purity). LCMS: m/z found 438.19 [M+H]⁺, rt=2.56 min (Method B) [Waters Acquity BEH C8 column (1.7 μm, 50×2.1 mm)]; ¹H NMR (400 MHz, Chloroform-d): δ 9.20 (d, J=2.2 Hz, 1H), 9.02 (d, J=1.9 Hz, 1H), 8.32 (t, J=2.2 Hz, 1H), 7.85 (dd, J=1.6, 7.1 Hz, 1H), 7.78 (d, J=7.4 Hz, 1H), 7.72-7.60 (m, 2H), 6.30 (q, J=8.0 Hz, 1H), 3.39 (q, J=6.9 Hz, 2H), 0.67 (t, J=7.0 Hz, 3H).

### Example 249 and Example 250

### (S)-5-cyano-N-(1-(3,4-difluorophenyl)-2,2,2-trifluoroethyl)-N-ethylpyridine-3-sulfonamide (Example 249) and (R)-5-cyano-N-(1-(3,4-difluorophenyl)-2,2,2-trifluoroethyl)-N-ethylpyridine-3-sulfonamide (Example 250)

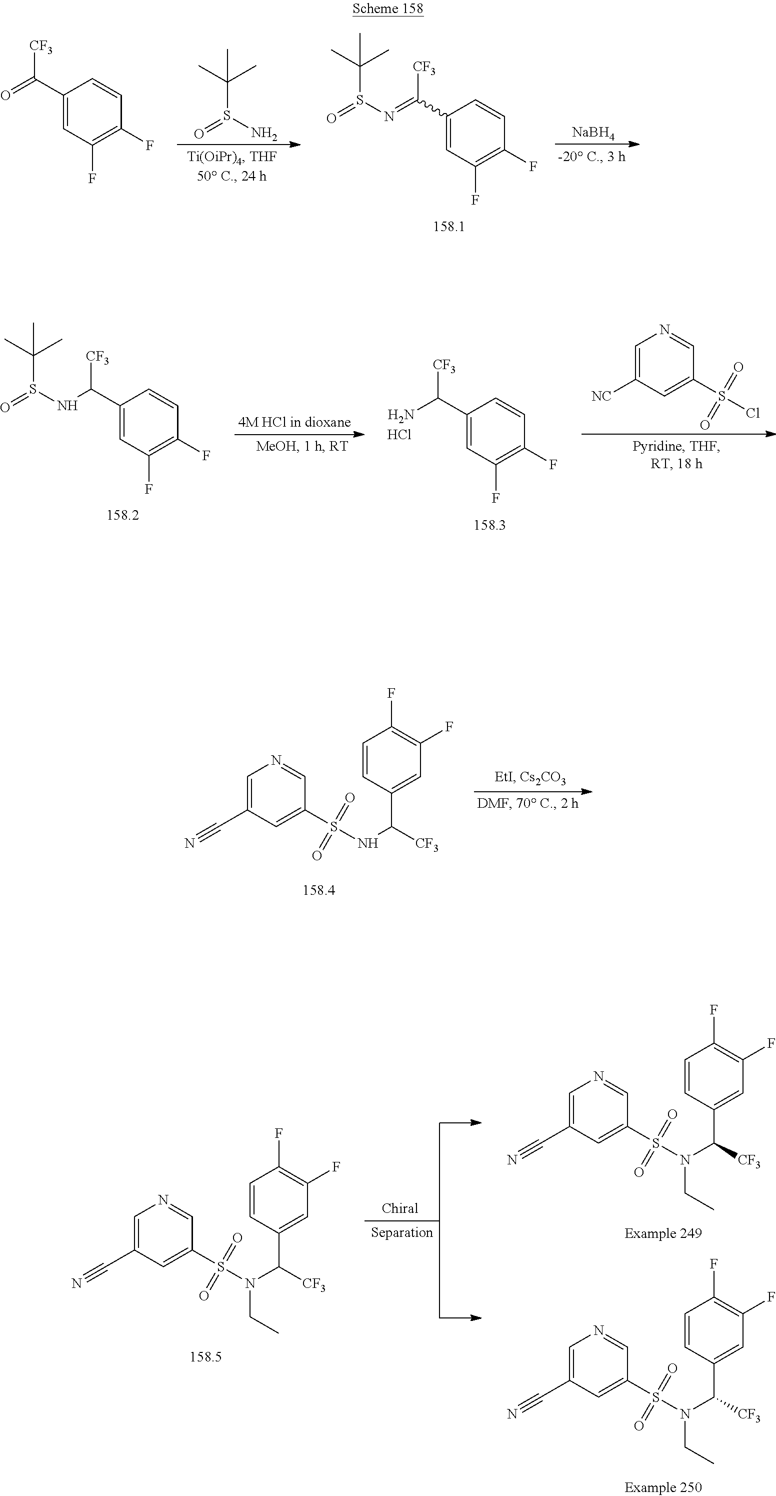

Synthesis of N-(1-(3,4-difluorophenyl)-2,2,2-trifluoroethylidene)-2-methylpropane-2-sulfinamide 158.1: Intermediate 158.1 was synthesized from 1-(3,4-difluorophenyl)-2,2,2-trifluoroethan-1-one following the protocol as described in Method P-I. The reaction mass was used in the forwarding step without any work up.

Synthesis of N-(1-(3,4-difluorophenyl)-2,2,2-trifluoroethyl)-2-methylpropane-2-sulfinamide 158.2: Intermediate 158.2 was synthesized from intermediate 158.1 following the protocol as described in Method Y. The compound was purified by column chromatography over silica gel using 45% ethyl acetate in hexane and isolated as colourless gum (1.6 g, 55% yield). ¹H NMR (400 MHz, DMSO-d₆): δ 7.73-7.60 (m, 1H), 7.54-7.40 (m, 2H), 6.70 (d, J=10 Hz, 1H), 5.35-5.29 (m, 1H), 1.05 (s, 9H).

Synthesis of 1-(3,4-difluorophenyl)-2,2,2-trifluoroethan-1-amine 158.3: Intermediate 158.2 (1.60 g) was dissolved in MeOH (10 ml) and cooled. 4 M HCl in dioxane (10 mL) was added to it and the reaction mixture was stirred at room temperature for 1 h. The volatiles were evaporated under reduced pressure and the crude compound 158.3 was used in the forwarding step without further purification. ¹H NMR (400 MHz, DMSO-d₆): δ 9.67-9.56 (brs, 1H), 7.83 (t, J=9.4 Hz, 1H), 7.70-7.60 (m, 1H), 7.60-7.50 (m, 1H), 5.65-5.55 (m, 1H).

Synthesis of 5-cyano-N-(1-(3,4-difluorophenyl)-2,2,2-trifluoroethyl)pyridine-3-sulfonamide 158.4: Intermediate 158.4 was synthesized from 5-cyanopyridine-3-sulfonyl chloride following the protocol as described in Method O using 1-(3,4-difluorophenyl)-2,2,2-trifluoroethan-1-amine hydrochloride. The compound was purified by column chromatography over silica gel using 25% ethyl acetate in hexane and isolated as colourless gum (650 mg, 80% yield). ¹H NMR (400 MHz, DMSO-d₆): 9.90-9.80 (brs, 1H), 9.17 (d, J=1.6 Hz, 1H), 9.02 (d, J=1.9 Hz, 1H), 8.48 (s, 1H), 7.55-7.40 (m, 1H), 7.40-7.35 (m, 1H), 7.35-7.25 (m, 1H), 5.58-5.55 (m, 1H).

Synthesis of 5-cyano-N-(1-(3,4-difluorophenyl)-2,2,2-trifluoroethyl)-N-ethylpyridine-3-sulfonamide 158.5: The ethylation of intermediate 158.5 was performed following the protocol as described in Method D. The crude was purified by column chromatography over silica gel using 5:1 ethyl acetate-hexane to obtain compound 158.5 as a colorless gum (240 mg, 34% yield). LCMS: m/z found 406.16 [M+H]⁺, rt=3.33 min (Method 2) [Waters Acquity BEH C8 column (1.7 μm, 50×2.1 mm)].

Chiral separation of racemic compound 158.5 provide both the enantiomers. Chiral separation method: Chiral separation was done on THAR-SFC-80 instrument by using CHIRALPAK IG column (21 mm×25 cm), 5μ, operating at 35° C. temperature, maintaining flow rate of 50 ml/min, using 90% CO₂ in super critical state and 10% of methanol as mobile phase, run this isocratic mixture up to 9 minutes and also maintained the isobaric condition of 100 bar at 220 nm wavelength.

Example 249: Colorless sticky gum (22 mg, 99.94% purity). LCMS: m/z found 406.16 [M+H]⁺, rt=3.33 min (Method 2) [Waters Acquity BEH C8 column (1.7 μm, 50×2.1 mm)]; ¹H NMR (400 MHz, Chloroform-d) δ 9.24 (d, J=2.1 Hz, 1H), 9.05 (d, J=1.8 Hz, 1H), 8.39 (t, J=1.9 Hz, 1H), 7.42-7.32 (m, 1H), 7.32-7.20 (m, 2H), 5.76 (q, J=8.2 Hz, 1H), 3.50-3.36 (m, 1H), 3.28-3.14 (m, 1H), 0.96 (t, J=7.1 Hz, 3H).

Example 250: Colorless sticky gum (46 mg, 98.86% purity). LCMS: m/z found 406.16 [M+H]⁺, rt=3.33 min (Method 2) [Waters Acquity BEH C8 column (1.7 μm, 50×2.1 mm)]; ¹H NMR (400 MHz, Chloroform-d) δ 9.24 (d, J=2.1 Hz, 1H), 9.07 (d, J=1.8 Hz, 1H), 8.39 (t, J=2.0 Hz, 1H), 7.42-7.32 (m, 1H), 7.32-7.20 (m, 2H), 5.76 (q, J=8.2 Hz, 1H), 3.50-3.36 (m, 1H), 3.28-3.14 (m, 1H), 0.96 (t, J=7.1 Hz, 3H).

### Example 251 and Example 252

### (R)—N-(1-(3-chlorophenyl)-2,2,2-trifluoroethyl)-5-cyano-N-ethylpyridine-3-sulfonamide (Example 251) and (S)—N-(1-(3-chlorophenyl)-2,2,2-trifluoroethyl)-5-cyano-N-ethylpyridine-3-sulfonamide (Example 252)

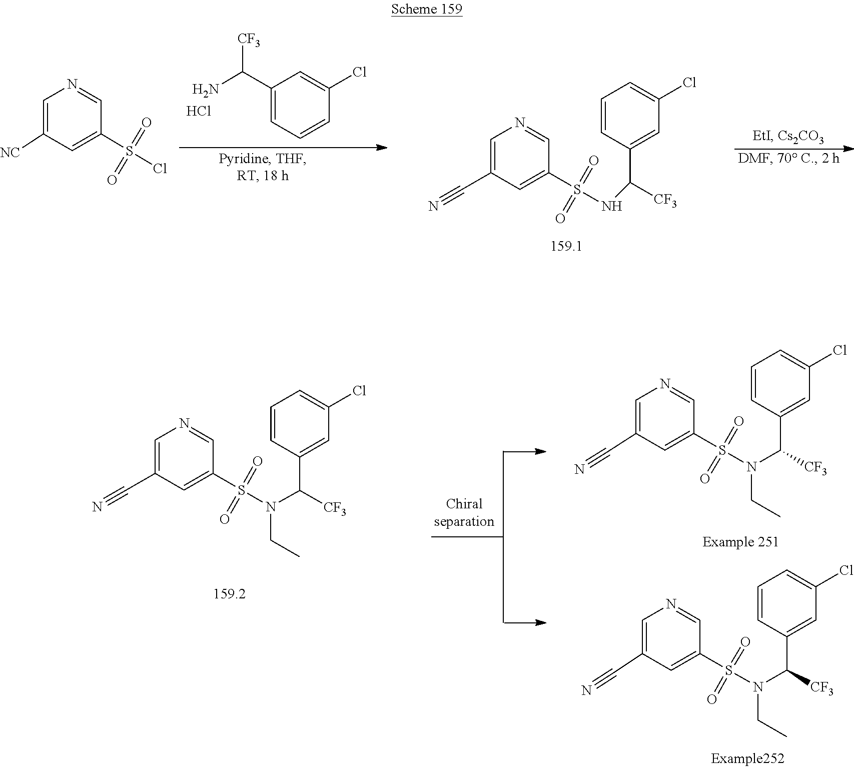

Synthesis of N-(1-(3-chlorophenyl)-2,2,2-trifluoroethyl)-5-cyanopyridine-3-sulfonamide 5.1: Sulfonamide 159.1 was synthesized from 5-cyanopyridine-3-sulfonyl chloride following the protocol as described in Method O using 1-(3-chlorophenyl)-2,2,2-trifluoroethan-1-amine hydrochloride. The compound was purified by column chromatography over silica gel using 30% ethyl acetate in hexane to isolate desired compound 159.1 as a yellow gum (400 mg, 60% yield). LCMS: m/z found 376.0 [M+H]⁺, rt=3.84 min (Method 3) [YMC Triart C18 column (3 μm, 33×2.1 mm)].

Synthesis of N-(1-(3-chlorophenyl)-2,2,2-trifluoroethyl)-5-cyano-N-ethylpyridine-3-sulfonamide 159.2: The ethylation of intermediate 159.1 was preformed following the protocol as described in Method D. The crude was purified by column chromatography over silica gel using 20% ethyl acetate-hexane to obtain compound 159.2 as a colorless gum (140 mg, 35% yield). LCMS: m/z found 404.14 [M+H]⁺, rt=3.22 min (Method 2) [Waters Acquity BEH C8 column (1.7 μm, 50×2.1 mm)]. Chiral separation of racemic compound 5.2 provide both the enantiomers. Chiral separation Method: Chiral separation was done on Agilent 1200 series instrument. Column name: Chiralpak IG (250×21 mm), 5μ, operating at ambient temperature and flow rate of 21.0 mL/min. Mobile phase: the mixture of 90% Hexane and 0.3% Isopropanol, held this isocratic mixture up to 25 min with wavelength of 246 nm.

Example 251: Brown sticky gum (52 mg, 99.39% purity). LCMS: m/z found 404.14 [M+H]⁺, rt=3.22 min (Method 2) [Waters Acquity BEH C8 column (1.7 μm, 50×2.1 mm)]; ¹H NMR (400 MHz, Chloroform-d) δ 9.23 (d, J=2.3 Hz, 1H), 9.05 (d, J=2.2 Hz, 1H), 8.36 (s, 1H), 7.47-7.37 (m, 4H), 5.77 (q, J=8.2 Hz, 1H), 3.49-3.38 (m, 1H), 3.29-3.18 (m, 1H), 0.98 (t, J=7.2 Hz, 3H).

Example 252: Brown sticky gum (35 mg, 98.11% purity). LCMS: m/z found 404.14 [M+H]⁺, rt=3.22 min (Method 2) [Waters Acquity BEH C8 column (1.7 μm, 50×2.1 mm)]; ¹H NMR (400 MHz, Chloroform-d) δ 9.23 (d, J=2.3 Hz, 1H), 9.05 (s, 1H), 8.36 (s, 1H), 7.47-7.37 (m, 4H), 5.77 (q, J=8.1 Hz, 1H), 3.69-3.33 (m, 1H), 3.31-3.14 (m, 1H), 0.98 (t, J=7.0 Hz, 3H).

### Example 253 and Example 254

### (S)—N-(1-(3-chloro-4-fluorophenyl)-2,2,2-trifluoroethyl)-5-cyano-N-ethylpyridine-3-sulfonamide (Example 253) and (R)—N-(1-(3-chloro-4-fluorophenyl)-2,2,2-trifluoroethyl)-5-cyano-N-ethylpyridine-3-sulfonamide (Example 254)

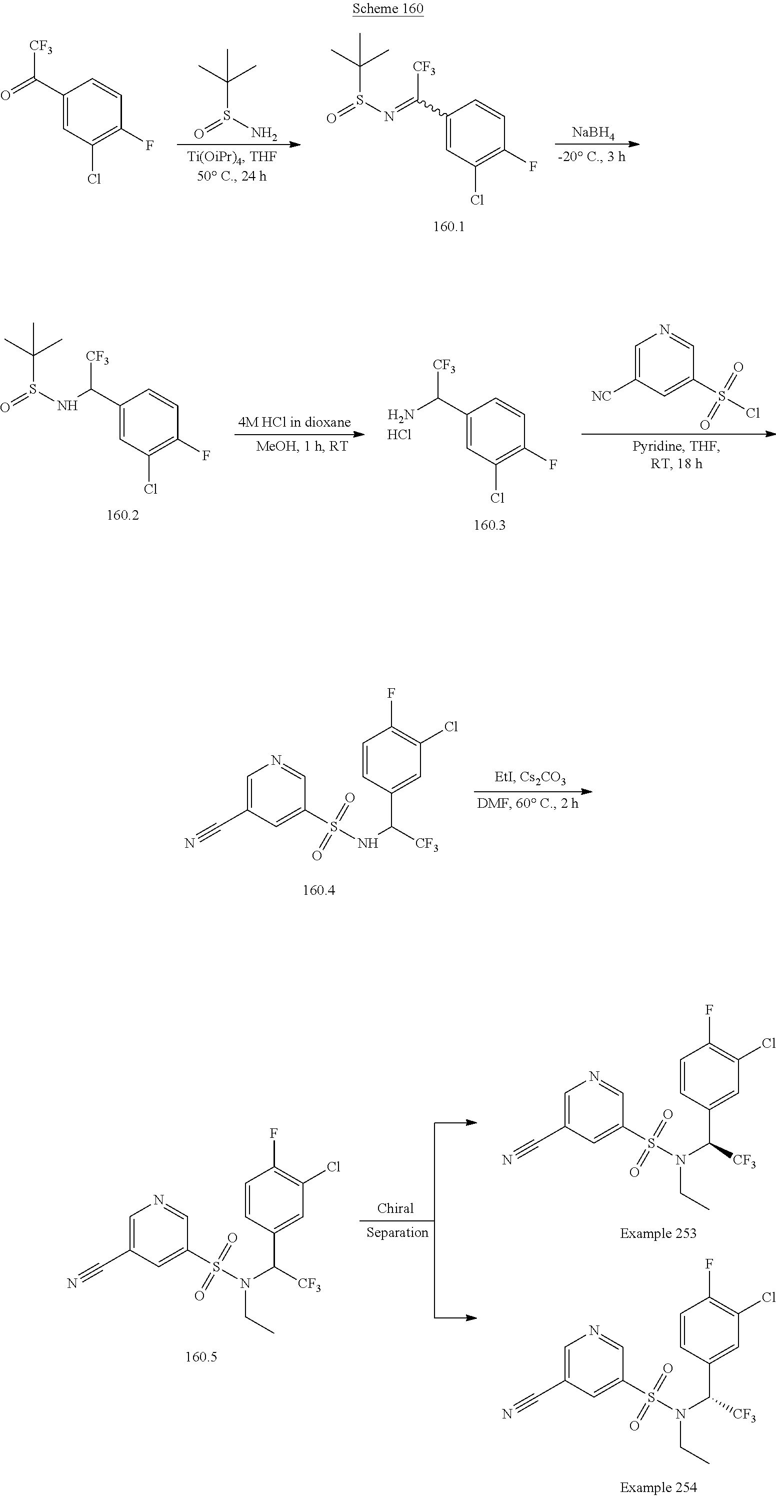

Synthesis of N-(1-(3-chloro-4-fluorophenyl)-2,2,2-trifluoroethylidene)-2-methylpropane-2-sulfinamide 160.1: Intermediate 16.1 was synthesized from 1-(3-chloro-4-fluorophenyl)-2,2,2-trifluoroethan-1-one following the protocol as described in Method P-I. The reaction mass was used in the forwarding step without any work up.

Synthesis of N-(1-(3-chloro-4-fluorophenyl)-2,2,2-trifluoroethyl)-2-methylpropane-2-sulfinamide 160.2: Intermediate 160.2 was synthesized from intermediate 160.1 following the protocol as described in Method Y. The compound was purified by column chromatography over silica gel using 45% ethyl acetate in hexane and isolated as a colourless gum (1.4 g, 48% yield). ¹H NMR (400 MHz, DMSO-d₆): δ 7.86 (d, J=6.0 Hz, 1H), 7.65-7.60 (m, 1H), 7.51-7.48 (m, 1H), 6.72 (d, J=10.0 Hz, 1H), 5.35-5.30 (m, 1H), 1.05 (s, 3H).

Synthesis of 1-(3-chloro-4-fluorophenyl)-2,2,2-trifluoroethan-1-amine 160.3: Intermediate 160.2 (1.40 g) was dissolved in MeOH (10 ml) and cooled. 4 M HCl in dioxane (10 mL) was added to it and the reaction mixture was stirred at room temperature for 1 h. The volatiles were evaporated under reduced pressure and the crude compound 160.3 was used in the forwarding step without further purification.

Synthesis of N-(1-(3-chloro-4-fluorophenyl)-2,2,2-trifluoroethyl)-5-cyanopyridine-3-sulfonamide 160.4: Intermediate 160.4 was synthesized from 5-cyanopyridine-3-sulfonyl chloride following the protocol as described in Method O using 1-(3-chloro-4-fluorophenyl)-2,2,2-trifluoroethan-1-amine hydrochloride. The compound was purified by column chromatography over silica gel using 25% ethyl acetate in hexane and isolated as colourless gum (650 mg, 80% yield). ¹H NMR (400 MHz, DMSO-d₆): δ 9.82 (s, 1H), 9.17 (s, 1H), 9.00 (s, 1H), 8.47 (s, 1H), 7.63 (d, J=6.5 Hz, 1H), 7.46-7.35 (m, 2H), 5.60-5.50 (m, 1H).

Synthesis of N-(1-(3-chloro-4-fluorophenyl)-2,2,2-trifluoroethyl)-5-cyano-N-ethylpyridine-3-sulfonamide 160.5: The ethylation of intermediate 160.5 was preformed following the protocol as described in Method D. The crude was purified by column chromatography over silica gel using 5:1 ethyl acetate-hexane to obtain compound 160.5 as a colorless gum (172 mg, 25% yield). LCMS: m/z found 422.13 [M+H]⁺, rt=3.24 min (Method 2) [Waters Acquity BEH C8 column (1.7 μm, 50×2.1 mm)]; Chiral separation of racemic compound 160.5 provide both the enantiomers. Chiral separation was done on THAR-SFC-80 instrument by using CHIRALPAK IG column (21 mm×25 cm), 5μ, operating at 35° C. temperature, maintaining flow rate of 60 ml/min, using 80% C02 in super critical state & 20% of (hexane:methanol:isopropanol—70:20:10) as mobile phase, run this isocratic mixture up to 14 minutes and also maintained the isobaric condition of 100 bar at 228 nm wavelength.

Example 253: Colorless sticky gum (20 mg, 99.09% purity). LCMS: m/z found 422.14 [M+H]⁺, rt=3.24 min (Method 2) [Waters Acquity BEH C8 column (1.7 μm, 50 15×2.1 mm)]; ¹H NMR (400 MHz, Chloroform-d): δ 9.24 (s, 1H), 9.06 (s, 1H), 8.39 (s, 1H), 7.54 (d, J=5.4 Hz, 1H), 7.48-7.37 (m, 1H), 7.27-7.18 (m, 1H), 5.76 (q, J=7.5 Hz, 1H), 3.48-3.36 (m, 1H), 3.28-3.16 (m, 1H), 0.98 (t, J=7.0 Hz, 3H).

Example 254: Colorless sticky gum (18 mg, 98.09% purity). LCMS: m/z found m/z found 422.12 [M+H]⁺, rt=3.25 min (Method B) [Waters Acquity BEH C8 column (1.7 μm, 50×2.1 mm)]; ¹H NMR (400 MHz, Chloroform-d): δ 9.24 (s, 1H), 9.09-9.03 (m, 1H), 8.39 (s, 1H), 7.54 (d, J=5.7 Hz, 1H), 7.48-7.36 (m, 1H), 7.28-7.18 (m, 1H), 5.83-5.69 (m, 1H), 3.49-3.38 (m, 1H), 3.26-3.16 (m, 1H), 0.98 (t, J=7.1 Hz, 3H).

### Example 255

### 5-cyano-N-methyl-N-(2,2,2-trifluoro-1-(3-fluoro-4-(trifluoromethyl)phenyl)ethyl)pyridine-3-sulfonamide

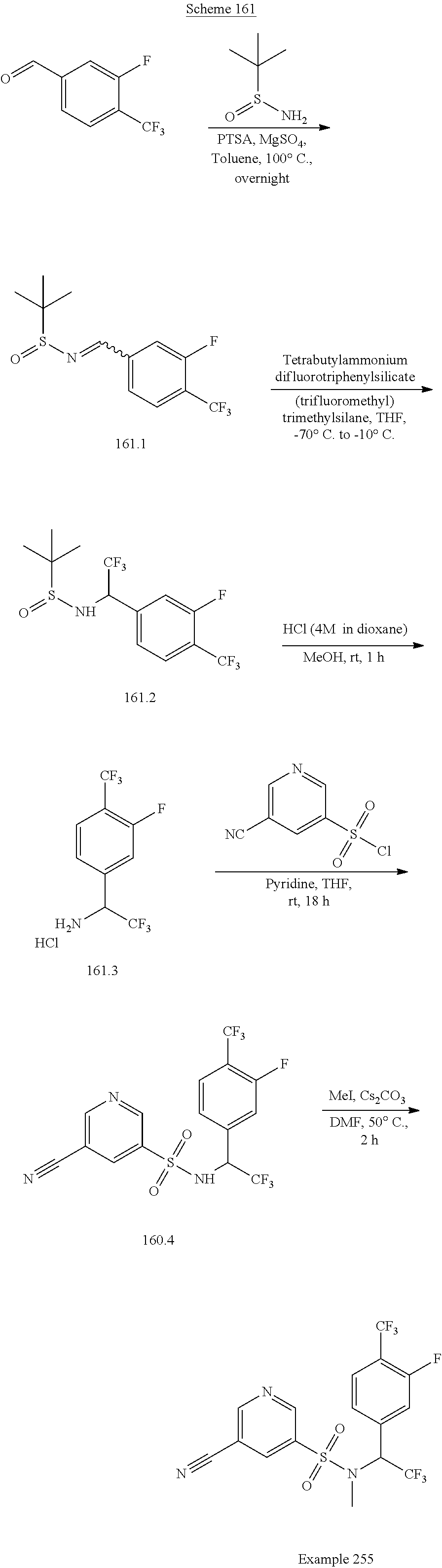

Synthesis of N-(3-fluoro-4-(trifluoromethyl)benzylidene)-2-methyl propane-2-sulfinamide 161.1: Intermediate 161.1 was synthesized from 3-fluoro-4-(trifluoromethyl)benzaldehyde following the protocol as described in Method P-II. The crude compound was purified by column chromatography over silica gel using 10% ethyl acetate in hexane to isolate desired compound 161.1 as a yellow gum (2.0 g, 65% yield). ¹H NMR (400 MHz, DMSO-d₆): δ 8.66 (s, 1H), 8.06 (d, J=11.6 Hz, 1H), 8.0-7.95 (m, 2H).

Synthesis of 2-methyl-N-(2,2,2-trifluoro-1-(3-fluoro-4-(trifluoromethyl)phenyl)ethyl)propane-2-sulfinamide 161.2: Intermediate 161.2 was synthesized from intermediate 161.1 following the protocol as described in Method Q-I. The crude compound was purified by column chromatography over silica gel using 10% ethyl acetate in hexane to isolate desired compound 161.2 as yellow gum (1.2 g, 47% yield). LCMS: m/z found 366.2 [M+H]⁺, rt=2.29 min (Method 12) [YMC Triart C18 column (3 μm, 33×2.1 mm)].

Synthesis of 2,2,2-trifluoro-1-(3-fluoro-4-(trifluoromethyl)phenyl)ethan-1-amine hydrochloride 161.3: Intermediate 161.2 (1.20 g) was dissolved in MeOH (10 ml) and cooled. 4 M HCl in dioxane (10 mL) was added to it and the reaction mixture was stirred at room temperature for 1 h. The volatiles were evaporated under reduced pressure and the crude compound 7.3 was used in the forwarding step without further purification. LCMS: m/z found 262.0 [M+H]⁺, rt=1.76 min (Method 3) [YMC Triart C18 column (3 μm, 33×2.1 mm)].

Synthesis of 5-cyano-N-(2,2,2-trifluoro-1-(3-fluoro-4-(trifluoromethyl)phenyl)ethyl)pyridine-3-sulfonamide 161.4: Intermediate 161.4 was synthesized from 5-cyanopyridine-3-sulfonyl chloride following the protocol as described in Method O using 2,2,2-trifluoro-1-(3-fluoro-4-(trifluoromethyl)phenyl)ethan-1-amine hydrochloride. The compound was purified by column chromatography over silica gel using 25% ethyl acetate in hexane and isolated as colourless gum (350 mg, 38% yield). LCMS: m/z found 428.0 [M+H]⁺, rt=1.91 min (Method 3) [YMC Triart C18 column (3 μm, 33×2.1 mm)].

Synthesis of 5-cyano-N-methyl-N-(2,2,2-trifluoro-1-(3-fluoro-4-(trifluoromethyl)phenyl)ethyl)pyridine-3-sulfonamide: The methylation of intermediate 161.4 was preformed following the protocol as described in Method D. The crude was purified by column chromatography over silica gel using 1:5 ethyl acetate-hexane to obtain compound Example 255 as a light-yellow sticky gum (20 mg, 97.61% purity, 22% yield). LCMS: m/z found 442.0 [M+H]⁺, rt=1.98 min (Method 3) [YMC Triart C18 column (3 μm, 33×2.1 mm)]; ¹H NMR (400 MHz, Chloroform-d): δ 9.23 (d, J=1.9 Hz, 1H), 9.09 (d, J=1.8 Hz, 1H), 8.39 (s, 1H), 7.73 (t, J=7.7 Hz, 1H), 7.43-7.32 (m, 2H), 5.90 (q, J=7.9 Hz, 1H), 2.80 (s, 3H).

### Example 256 and Example 257

### (S)—N-ethyl-5-fluoro-N-(2,2,2-trifluoro-1-(4-methoxyphenyl)ethyl)pyridine-3-sulfonamide (Example 256) and (R)—N-ethyl-5-fluoro-N-(2,2,2-trifluoro-1-(4-methoxyphenyl)ethyl)pyridine-3-sulfonamide (Example 257)

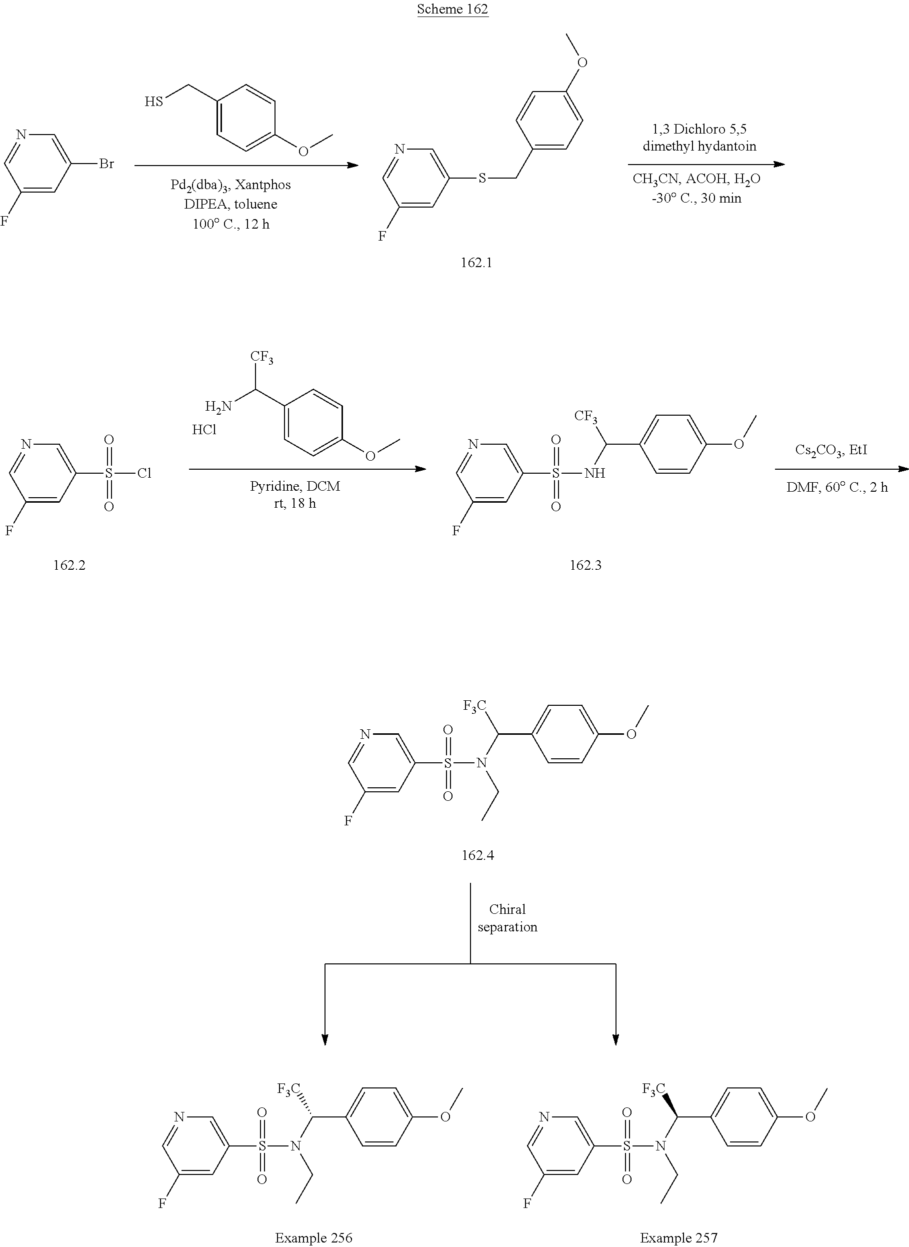

Synthesis of 3-fluoro-5-((4-methoxybenzyl)thio)pyridine 162.1: Intermediate 162.1 was synthesized from 3-bromo-5-fluoropyridine following a method analogous to that described in Method I using (4-methoxyphenyl)methanethiol. The crude was purified by column chromatography over silica gel using 20% ethyl acetate-hexane to yield compound 162.1 as yellow gum (2.5 g, 86% yield). ¹H NMR (400 MHz, DMSO-d₆): δ 8.36-8.33 (m, 2H), 7.81-7.76 (m, 1H), 7.31-7.26 (m, 2H), 6.89-6.84 (m, 2H), 4.30 (s, 2H), 3.71 (s, 3H).

Synthesis of 5-fluoropyridine-3-sulfonyl chloride 162.2: Sulfonyl chloride 162.2 was synthesized from intermediate 162.1 following the procedure described in Method T. The crude sulfonyl chloride was used immediately in the forwarding step without further purification.

Synthesis of 5-fluoro-N-(2,2,2-trifluoro-1-(4-methoxyphenyl)ethyl)pyridine-3-sulfonamide 162.3: Sulfonamide 162.3 was synthesized from sulfonyl chloride 162.2 following the protocol as described in Method O using 2,2,2-trifluoro-1-(4-methoxyphenyl)ethan-1-amine hydrochloride. The compound was purified by column chromatography over silica gel using 15% ethyl acetate in hexane to isolate desired compound 162.3 as a yellow gum (220 mg, 30% yield). LCMS: m/z found 365.0 [M+H]⁺, rt=1.78 min (Method 3) [YMC Triart C18 column (3 μm, 33×2.1 mm)].

Synthesis of N-ethyl-5-fluoro-N-(2,2,2-trifluoro-1-(4-methoxyphenyl)ethyl)pyridine-3-sulfonamide 162.4: The ethylation of intermediate 162.3 was preformed following the protocol as described in Method D at 60° C. The crude was purified by column chromatography over silica gel using 5:1 ethyl acetate-hexane to obtain compound 162.4 as a colorless gum (90 mg, 38% yield). LCMS: m/z found 393.16 [M+H]⁺, rt=3.16 min (Method 2) [Waters Acquity BEH C8 column (1.7 μm, 50×2.1 mm)]. Chiral separation of racemic compound 162.4 provide both enantiomers. Chiral separation method: Preparative SFC was done in waters sfc-80 instrument equipped with waters UV Detector w2489 by using Column: Reflect C-amylose-A (30 mm×250 mm), 5 μm, operating at 35° C. temperature, maintaining flow rate of 60 gm/min, using 60% C02 in super critical state and 40% of (MeOH) as mobile phase, running this isocratic mixture up to 7 minutes and also maintained the isobaric condition of 100 bar at 228 nm wavelength.

Example 256: Colorless sticky gum (45 mg, 96.99% purity). LCMS: m/z found 393.16 [M+H]⁺, rt=3.18 min (Method 2) [Waters Acquity BEH C8 column (1.7 μm, 50×2.1 mm)]. ¹H NMR (400 MHz, Chloroform-d): δ 8.90 (s, 1H), 8.70-8.63 (m, 1H), 7.86-7.78 (m, 1H), 7.43-7.34 (m, 2H), 6.96-6.88 (m, 2H), 5.75 (q, J=8.2 Hz, 1H), 3.83 (s, 3H), 3.43-3.31 (m, 1H), 3.28-3.16 (m, 1H), 1.00-0.90 (m, 3H), 7.33-7.24 (m, 3H).

Example 257: Colorless sticky gum (50 mg, 98.66% purity). LCMS: m/z found 393.14 [M+H]⁺, rt=3.18 min (Method 2) [Waters Acquity BEH C8 column (1.7 μm, 50×2.1 mm)]. ¹H NMR (400 MHz, Chloroform-d): δ 8.90 (s, 1H), 8.66 (d, J=2.4 Hz, 1H), 7.86-7.78 (m, 1H), 7.38 (d, J=8.6 Hz, 2H), 6.91 (d, J=8.8 Hz, 2H), 5.75 (q, J=8.4 Hz, 1H), 3.81 (s, 3H), 3.43-3.29 (m, 1H), 3.30-3.16 (m, 1H), 0.94 (t, J=7.0 Hz, 3H).

### Example 258 and Example 259

### (S)-5-cyano-N-ethyl-N-(2,2,2-trifluoro-1-(4-fluoro-3-methoxyphenyl)ethyl)pyridine-3-sulfonamide (Example 258) and (R)-5-cyano-N-ethyl-N-(2,2,2-trifluoro-1-(4-fluoro-3-methoxyphenyl)ethyl)pyridine-3-sulfonamide (Example 259)

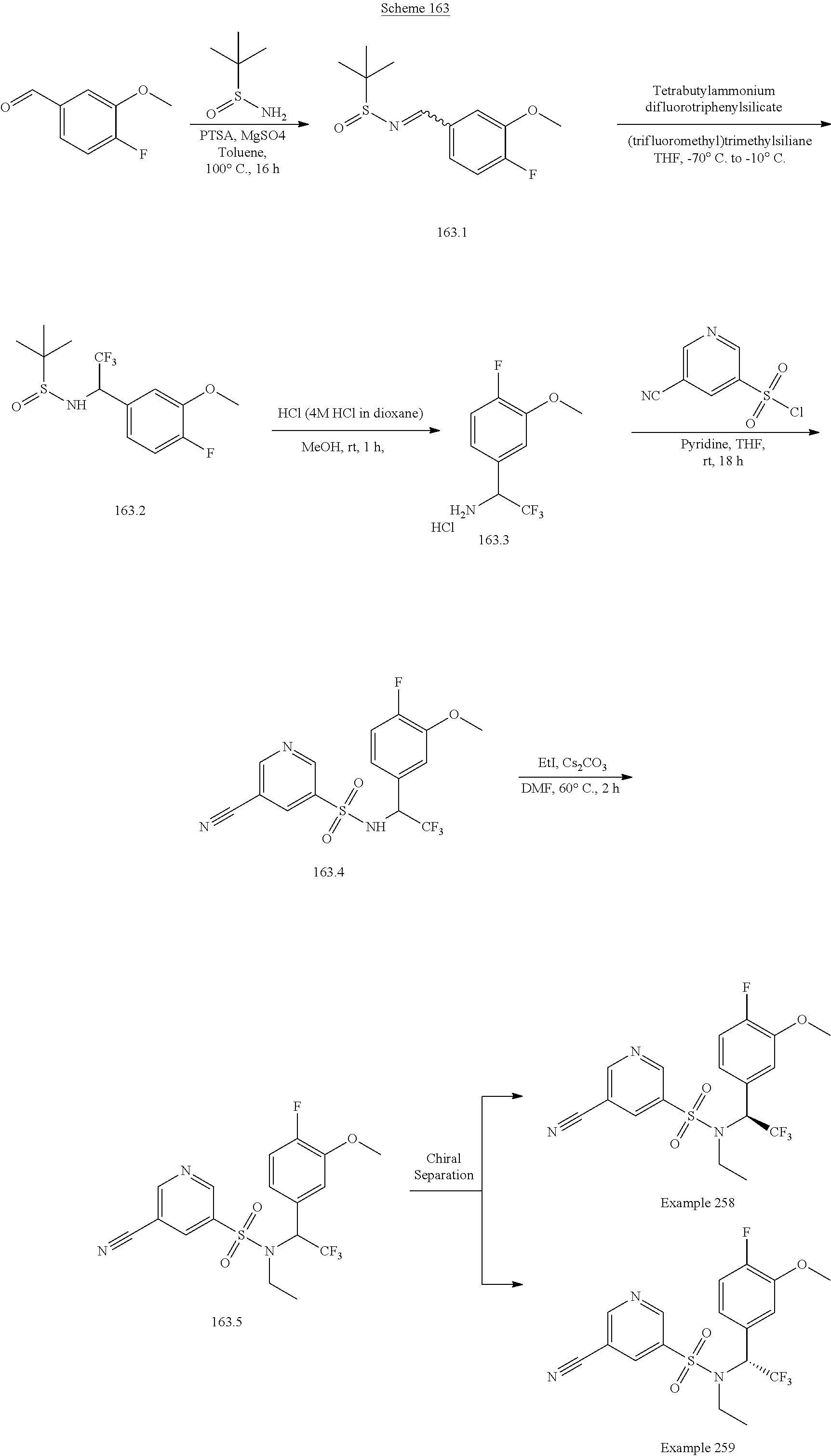

Synthesis of N-(4-fluoro-3-methoxybenzylidene)-2-methylpropane-2-sulfinamide 163.1: Intermediate 163.1 was synthesized from 4-fluoro-3-methoxybenzaldehyde following the protocol as described in Method P-II. The crude compound was purified by column chromatography over silica gel using 10% ethyl acetate in hexane to isolate desired compound 163.1 as a yellow gum (2.5 g, 73% yield). LCMS: m/z found 257.99 [M+H]⁺, rt=2.01 min (Method 31) [Waters Acquity BEH C18 column (1.7 μm, 50×2.1 mm)].

Synthesis of 2-methyl-N-(2,2,2-trifluoro-1-(4-fluoro-3-methoxyphenyl)ethyl)propane-2-sulfinamide 163.2: Intermediate 163.2 was synthesized from intermediate 163.1 following the protocol as described in Method Q-I. The crude compound was purified by column chromatography over silica gel using 10% ethyl acetate in hexane to isolate desired compound 162.2 as yellow gum (520 mg, 29% yield). LCMS: m/z found 327.97 [M+H]⁺, rt=2.16 min (Method 31) [Waters Acquity BEH C18 column (1.7 μm, 50×2.1 mm)].

Synthesis of 2,2,2-trifluoro-1-(4-fluoro-3-methoxyphenyl)ethan-1-amine hydrochloride 163.3: Intermediate 163.2 (600 mg) was dissolved in MeOH (6 ml) and cooled. 4 M HCl in dioxane (6 mL) was added to it and the reaction mixture was stirred at room temperature for 1 h. The volatiles were evaporated under reduced pressure and the crude compound 163.3 was used in the forwarding step without further purification. LCMS: m/z found 224.2 [M+H]⁺, rt=1.62 min (Method 12) [YMC Triart C18 column (3 μm, 33×2.1 mm)].

Synthesis of 5-cyano-N-(2,2,2-trifluoro-1-(4-fluoro-3-methoxyphenyl)ethyl)pyridine-3-sulfonamide 163.4: Intermediate 163.4 was synthesized from 5-cyanopyridine-3-sulfonyl chloride following the protocol as described in Method O using 2,2,2-trifluoro-1-(4-fluoro-3-methoxyphenyl)ethan-1-amine hydrochloride. The compound was purified by column chromatography over silica gel using 25% ethyl acetate in hexane and isolated as colourless gum (550 mg, 53% yield). LCMS: m/z found 390.0 [M+H]⁺, rt=1.79 min (Method 3) [YMC Triart C18 column (3 μm, 33×2.1 mm)].

Synthesis of 5-cyano-N-ethyl-N-(2,2,2-trifluoro-1-(4-fluoro-3-methoxyphenyl)ethyl)pyridine-3-sulfonamide 163.5: The ethylation of intermediate 163.4 was preformed following the protocol as described in Method D at 60° C. The crude was purified by column chromatography over silica gel using 5:1 ethyl acetate-hexane to obtain compound 163.5 as a colorless gum (200 mg, 40% yield). LCMS: m/z found 418.0 [M+H]⁺, rt=3.15 min (Method 3) [YMC Triart C18 column (3 μm, 33×2.1 mm)]. Preparative SFC was done in Waters SFC-80 instrument equipped with waters UV Detector w2489 by using a Chiralpak IG (21 mm×250 mm) column 5 μm, operating at 35° C. temperature, maintaining flow rate of 60 gm/min, using 80% CO² in super critical state and 20% of MeOH as mobile phase, running this isocratic mixture up to 7 minutes and also maintained the isobaric condition of 100 bar at 220 nm wavelength.

Example 258: Yellow sticky gum (40 mg, 99.26% purity). LCMS: m/z found 418.17 [M+H]⁺, rt=3.15 min (Method 2) [Waters Acquity BEH C8 column (1.7 μm, 50×2.1 mm)]. ¹H NMR (400 MHz, DMSO-d₆): δ 9.38 (d, J=1.6 Hz, 1H), 9.30 (s, 1H), 8.96 (s, 1H), 7.26 (dd, J=8.6, 11.0 Hz, 1H), 7.13-6.95 (m, 2H), 6.02 (q, J=8.2 Hz, 1H), 3.74 (s, 3H), 3.52-3.40 (m, 1H), 3.39-3.30 (m, 1H), 1.02 (t, J=6.8 Hz, 3H).

Example 259: Yellow sticky gum (50 mg, 97.90% purity). LCMS: m/z found 418.17 [M+H]⁺, rt=3.14 min (Method 2) [Waters Acquity BEH C8 column (1.7 μm, 50×2.1 mm)]. ¹H NMR (400 MHz, DMSO-d₆): δ 9.38 (d, J=1.9 Hz, 1H), 9.30 (d, J=1.3 Hz, 1H), 8.96 (s, 1H), 7.26 (dd, J=8.6, 11.1 Hz, 1H), 7.11-6.99 (m, 2H), 6.02 (q, J=8.7 Hz, 1H), 3.74 (s, 3H), 3.54-3.40 (m, 1H), 3.40-3.32 (m, 1H), 1.02 (t, J=6.8 Hz, 3H).

### Example 260 to Example 263

### (S)—N-ethyl-5-fluoro-N-(2,2,2-trifluoro-1-(4-fluorophenyl)ethyl)pyridine-3-sulfonamide (Example 260), (R)—N-ethyl-5-fluoro-N-(2,2,2-trifluoro-1-(4-fluorophenyl)ethyl)pyridine-3-sulfonamide (Example 261), (R)—N-(1-(4-chlorophenyl)-2,2,2-trifluoroethyl)-5-fluoro-N-methylpyridine-3-sulfonamide (Example 262) and (R)-5-fluoro-N-methyl-N-(2,2,2-trifluoro-1-(4-methoxyphenyl)ethyl)pyridine-3-sulfonamide (Example 263)

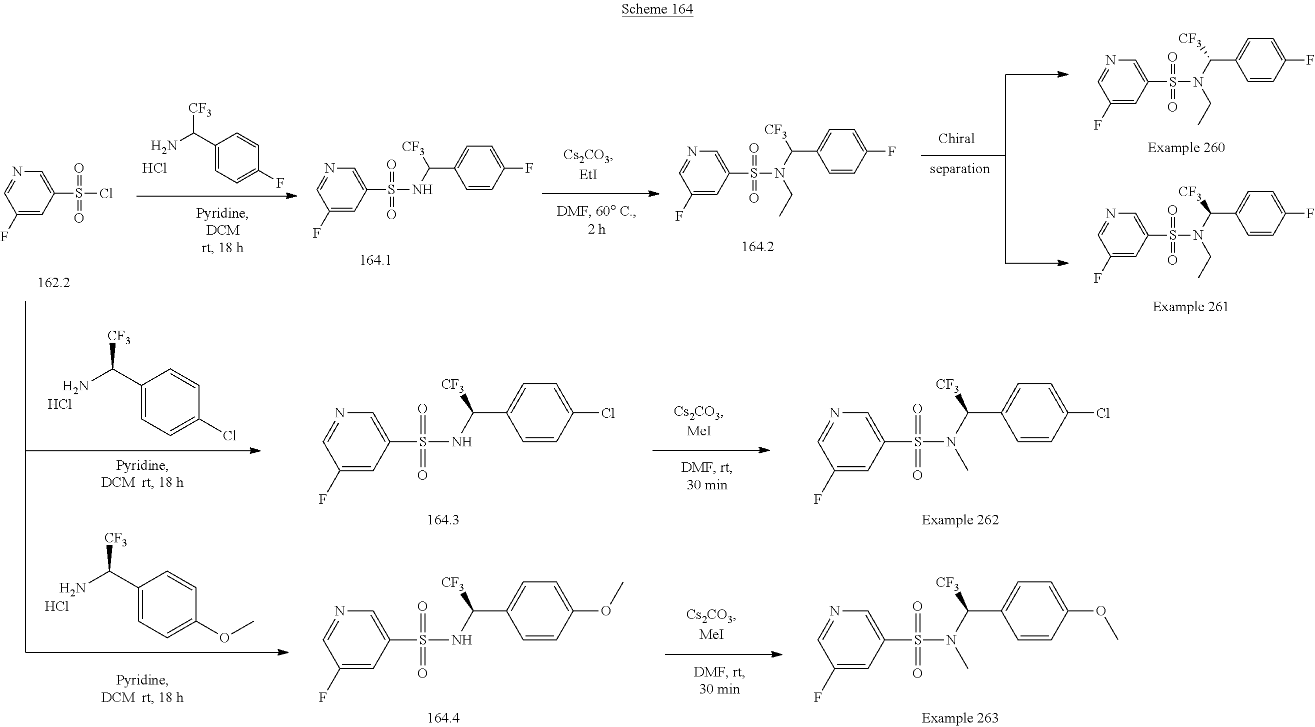

Synthesis of 5-fluoro-N-(2,2,2-trifluoro-1-(4-fluorophenyl)ethyl)pyridine-3-sulfonamide 164.1: Sulfonamide 164.1 was synthesized from 5-fluoropyridine-3-sulfonyl chloride 162.2 following the protocol as described in Method O using (R)-1-(4-fluorophenyl)-2,2,2-trifluoroethan-1-amine hydrochloride. The compound was purified by column chromatography over silica gel using 20% ethyl acetate in hexane to isolate desired compound 164.1 as yellow gum (220 mg, 40% yield). LCMS: m/z found 353.0[M+H]⁺, rt=1.80 min (Method 3) [YMC Triart C18 column (3 μm, 33×2.1 mm)].

Synthesis of N-ethyl-5-fluoro-N-(2,2,2-trifluoro-1-(4-fluorophenyl)ethyl)pyridine-3-sulfonamide 164.2: The ethylation of intermediate 164.1 was preformed following the protocol as described in Method D at 60° C. The crude was purified by column chromatography over silica gel using 5:1 ethyl acetate-hexane to obtain compound 164.2 as colorless gum (175 mg, 38% yield). LCMS: m/z found 381.0 [M+H]⁺, rt=1.96 min (Method 3) [YMC Triart C18 column (3 μm, 33×2.1 mm)]. Preparative SFC was done in waters sfc-80 instrument equipped with waters UV Detector w2489 by using Column: Chiralpak IG (21 mm×250 mm), 5 μm, operating at 35° C. temperature, maintaining flow rate of 60 gm/min, using 80% C02 in super critical state and 20% of MeOH as mobile phase, running this isocratic mixture up to 7 minutes and also maintained the isobaric condition of 100 bar at 220 nm wavelength.

Example 260: Yellow sticky gum (32 mg, 97.82% purity). LCMS: m/z found 381.15 [M+H]⁺, rt=3.17 min (Method 2) [Waters Acquity BEH C8 column (1.7 μm, 50×2.1 mm)]. ¹H NMR (400 MHz, DMSO-d₆) δ 8.94 (s, 1H), 8.89 (d, J=2.3 Hz, 1H), 8.36-8.28 (m, 1H), 7.44 (t, J=5.7, 8.2 Hz, 2H), 7.23 (t, J=8.7 Hz, 2H), 6.06 (q, J=8.4 Hz, 1H), 3.49-3.23 (m, 2H), 0.95 (t, J=6.9 Hz, 3H).

Example 261: Yellow sticky gum (31 mg, 94.13% purity). LCMS: m/z found 381.11 [M+H]⁺, rt=3.17 min (Method 2) [Waters Acquity BEH C8 column (1.7 μm, 50×2.1 mm)]. ¹H NMR (400 MHz, DMSO-d₆) δ 8.94 (s, 1H), 8.89 (d, J=2.4 Hz, 1H), 8.36-8.28 (m, 1H), 7.44 (dd, J=5.2, 8.7 Hz, 2H), 7.23 (t, J=8.8 Hz, 2H), 6.05 (q, J=8.6 Hz, 1H), 3.47-3.29 (m, 2H), 0.95 (t, J=6.9 Hz, 3H).

Synthesis of (R)—N-(1-(4-chlorophenyl)-2,2,2-trifluoroethyl)-5-fluoropyridine-3-sulfonamide 164.3: Sulfonamide 164.3 was synthesized from 5-fluoropyridine-3-sulfonyl chloride following the protocol as described in Method O using (R)-1-(4-chlorophenyl)-2,2,2-trifluoroethan-1-amine hydrochloride. The compound was purified by column chromatography over silica gel using 20% ethyl acetate in hexane to isolate desired compound 164.2 as a yellow gum (400 mg, 58% yield). LCMS: m/z found 368.9 [M+H]⁺, rt=1.88 min (Method 3) [YMC Triart C18 column (3 μm, 33×2.1 mm)].

Synthesis of (R)—N-(1-(4-chlorophenyl)-2,2,2-trifluoroethyl)-5-fluoro-N-methylpyridine-3-sulfonamide: The methylation of intermediate 164.3 was performed following the protocol as described in Method D. The crude was purified by column chromatography over silica gel using 10% ethyl acetate in hexane to obtain compound Example 262 as a yellow sticky gum (62 mg, 99.52% purity). LCMS: m/z found 383.21 [M+H]⁺, rt 3.18 min (Method 2) [Waters Acquity BEH C8 column (1.7 μm, 50×2.1 mm)]; ¹H NMR (400 MHz, Chloroform-d): δ 8.89 (s, 1H), 8.70 (d, J=1.9 Hz, 1H), 7.82 (d, J=7.4 Hz, 1H), 7.40 (q, J=8.8 Hz, 4H), 5.83 (q, J=8.2 Hz, 1H), 2.74 (s, 3H).

Synthesis of (R)-5-fluoro-N-(2,2,2-trifluoro-1-(4-methoxyphenyl)ethyl)pyridine-3-sulfonamide 164.4: Sulfonamide 164.4 was synthesized from 5-fluoropyridine-3-sulfonyl chloride following the protocol as described in Method O using (R)-2,2,2-trifluoro-1-(4-methoxyphenyl)ethan-1-amine hydrochloride. The compound was purified by column chromatography over silica gel using 20% ethyl acetate in hexane to isolate desired compound 164.4 as yellow gum (400 mg, 58% yield). LCMS: m/z found 365.0 [M+H]1, rt=1.82 min (Method 3) [YMC Triart C18 column (3 μm, 33×2.1 mm)].

Synthesis of (R)-5-fluoro-N-methyl-N-(2,2,2-trifluoro-1-(4-methoxyphenyl)ethyl)pyridine-3-sulfonamide: The methylation of intermediate 164.4 was performed following the protocol as described in Method D. The crude was purified by column chromatography over silica gel using 10% ethyl acetate in hexane to obtain compound Example 263 as a light-yellow sticky gum (80 mg, 97.97% purity). LCMS: m/z found 379.14[M+H]⁺, rt=3.14 min (Method 2) [Waters Acquity BEH C8 column (1.7 μm, 50×2.1 mm)]; ¹H NMR (400 MHz, Chloroform-d): δ 8.88 (s, 1H), 8.68 (d, J=2.4 Hz, 1H), 7.86-7.76 (m, 1H), 7.33 (d, J=8.4 Hz, 2H), 6.92 (d, J=8.8 Hz, 2H), 5.80 (q, J=8.8 Hz, 1H), 3.82 (s, 3H), 2.75 (s, 3H).

### Example 264

### 5-Cyano-N-ethyl-N-(2,2,2-trifluoro-1-(5-methylthiazol-2-yl)ethyl)pyridine-3-sulfonamide

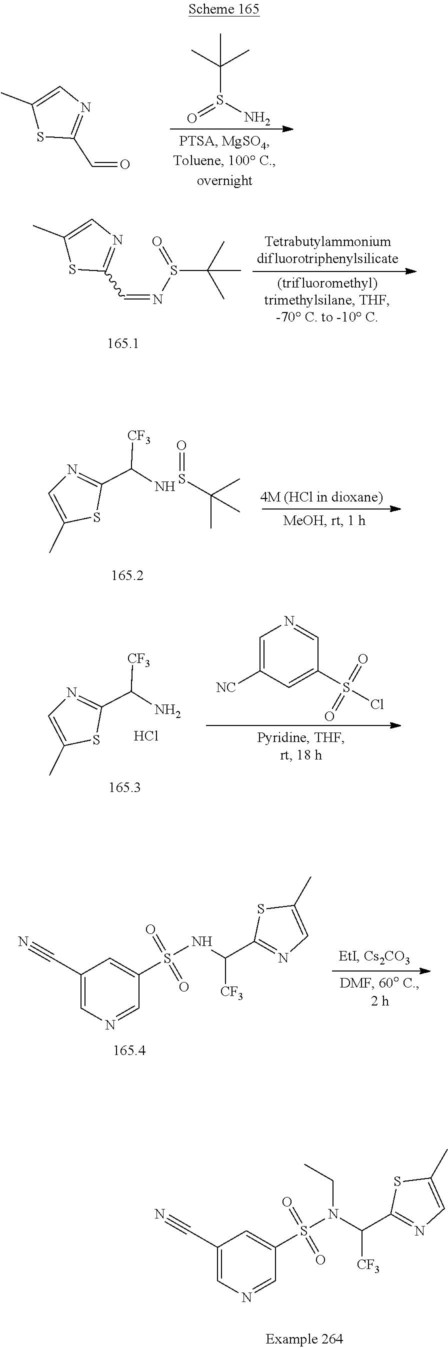

Synthesis of 2-methyl-N-((5-methylthiazol-2-yl)methylene)propane-2-sulfinamide 165.1: Intermediate 165.1 was synthesized from 5-methylthiazole-2-carbaldehyde following the protocol as described in Method P-II. The crude compound was purified by column chromatography over silica gel using 10% ethyl acetate in hexane to isolate desired compound 165.1 as a yellow oil (2.5 g, 69% yield). ¹H NMR (400 MHz, DMSO-d₆): δ 8.46 (s, 1H), 7.89 (s, 1H), 2.55 (s, 3H), 1.18 (s, 9H).

Synthesis of 2-methyl-N-(2,2,2-trifluoro-1-(5-methylthiazol-2-yl)ethyl)propane-2-sulfinamide 165.2: Intermediate 165.2 was synthesized from intermediate 165.1 following the protocol as described in Method Q-I. The crude compound was purified by column chromatography over silica gel using 10% ethyl acetate in hexane to isolate desired compound 165.2 as light-yellow gum (1.60 g, 79% yield). LCMS: m/z found 301.0 [M+H]⁺, rt=1.73 min (Method 3) [YMC Triart C18 column (3 μm, 33×2.1 mm)].

Synthesis of 2,2,2-trifluoro-1-(5-methylthiazol-2-yl)ethan-1-amine hydrochloride: Intermediate 165.2 (600 mg) was dissolved in MeOH (6 ml) and cooled. 4M HCl in dioxane (6 mL) was added to it and the reaction mixture was stirred at room temperature for 1 h. The volatiles were evaporated under reduced pressure and the crude compound 165.3 was used in the forwarding step without further purification. LCMS: m/z found 196.9 [M+H]1, rt=1.36 min (Method 3) [YMC Triart C18 column (3 μm, 33×2.1 mm)].

Synthesis of 5-cyano-N-(2,2,2-trifluoro-1-(5-methylthiazol-2-yl)ethyl)pyridine-3-sulfonamide 165.4: Sulfonamide 165.4 was synthesized from 5-cyanopyridine-3-sulfonyl chloride following the protocol as described in Method O using 2,2,2-trifluoro-1-(5-methylthiazol-2-yl)ethan-1-amine hydrochloride. The compound was purified by column chromatography over silica gel using 20% ethyl acetate in hexane to isolate desired compound 165.4 as yellow gum (300 mg, 33% yield). LCMS: m/z found 363.0 [M+H]⁺, rt=1.72 min (Method 3) [YMC Triart C18 column (3 μm, 33×2.1 mm)].

Synthesis of 5-cyano-N-ethyl-N-(2,2,2-trifluoro-1-(5-methylthiazol-2-yl)ethyl)pyridine-3-sulfonamide: The methylation of intermediate 165.4 was performed following the protocol as described in Method D. The crude was purified by column chromatography over silica gel using 10% ethyl acetate in hexane to obtain compound Example 264 as a light-yellow sticky gum (80 mg, 97.97% purity, 15% yield). LCMS: m/z found 391.14 [M+H]⁺, rt=3.08 min (Method 2) [Waters Acquity BEH C8 column (1.7 μm, 50×2.1 mm)]; ¹H NMR (400 MHz, Chloroform-d): δ 9.20 (d, J=2.0 Hz, 1H), 9.00 (d, J=1.4 Hz, 1H), 8.41 (s, 1H), 7.34 (s, 1H), 5.99 (q, J=7.7 Hz, 1H), 3.63-3.11 (m, 2H), 2.47 (s, 3H), 1.19 (t, J=7.0 Hz, 3H).

### Example 265

### (R)—N-ethyl-N-(2,2,2-trifluoro-1-(4-fluorophenyl)ethyl)pyrazine-2-sulfonamide

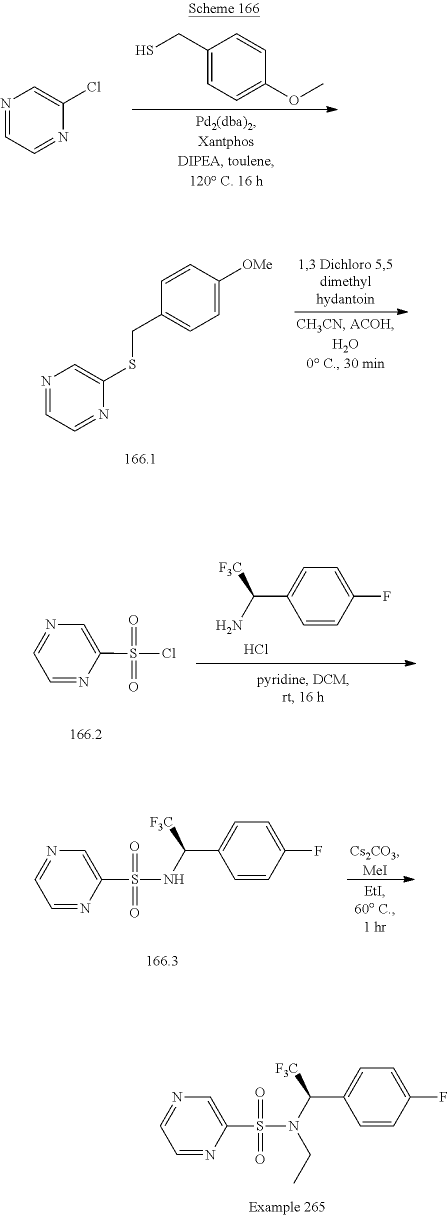

Synthesis of 2-((4-methoxybenzyl)thio)pyrazine 166.1: Intermediate 166.1 was synthesized from 2-chloropyrazine following a method analogous to that described in Method I using (4-methoxyphenyl)methanethiol. The crude was purified by column chromatography over silica gel using 20% ethyl acetate-hexane to yield compound 166.1 as yellow gum (2.50 g, 61% yield). LCMS: m/z found 233.0 [M+H]⁺, rt=1.85 min (Method 3) [YMC Triart C18 column (3 μm, 33×2.1 mm)].

Synthesis of pyrazine-2-sulfonyl chloride 166.2: Sulfonyl chloride 166.2 was synthesized from intermediate 166.1 following the procedure described in Method T. The crude sulfonyl chloride was used immediately in the forwarding step without further purification.

Synthesis of (R)—N-(2,2,2-trifluoro-1-(4-fluorophenyl)ethyl)pyrazine-2-sulfonamide 166.3: Sulfonamide 166.3 was synthesized from sulfonyl chloride 166.2 following the protocol as described in Method O using (R)-2,2,2-trifluoro-1-(4-fluorophenyl)ethan-1-amine hydrochloride. The compound was purified by column chromatography over silica gel using 20% ethyl acetate in hexane to isolate desired compound 166.3 as yellow gum (95 mg, 26% yield). LCMS: m/z found 336.1 [M+H]⁺, rt=1.74 min (Method 3) [YMC Triart C18 column (3 μm, 33×2.1 mm)].

Synthesis of (R)—N-ethyl-N-(2,2,2-trifluoro-1-(4-fluorophenyl)ethyl)pyrazine-2-sulfonamide: The ethylation of intermediate 1266.3 was preformed following the protocol as described in Method D. The crude was purified by column chromatography over silica gel using 20% ethyl acetate in hexane to obtain compound Example 265 as colourless sticky gum (35 mg, 98.66% purity, 34% yield). LCMS: m/z found 364.14 [M+H]⁺, rt=3.42 min (Method 2) [Waters Acquity BEH C8 column (1.7 μm, 50×2.1 mm)]. ¹H NMR (400 MHz, Chloroform-d): δ 9.19 (s, 1H), 8.79 (d, J=1.6 Hz, 1H), 8.64 (s, 1H), 7.52 (dd, J=5.1, 8.3 Hz, 2H), 7.09 (t, J=8.4 Hz, 2H), 5.79 (q, J=8.4 Hz, 1H), 3.58-3.39 (m, 2H), 0.95 (t, J=6.8 Hz, 3H).

### Example 266

### (R)—N,6-dimethyl-N-(2,2,2-trifluoro-1-(4-(trifluoromethyl)phenyl)ethyl)pyridine-3-sulfonamide

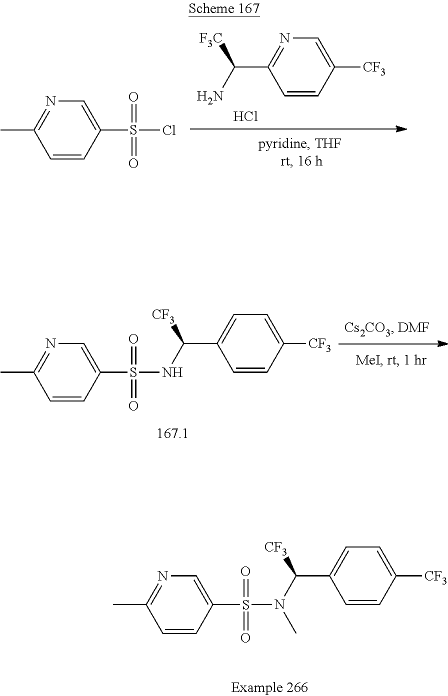 [SMILES: N[C@H](c1ccc(C(F)(F)F)cn1)C(F)(F)F ; Cc1ccc(S(=O)(=O)Cl)cn1 ; Cl ; Cc1ccc(S(=O)(=O)N[C@H](c2ccc(C(F)(F)F)cc2)C(F)(F)F)cn1 ; Cc1ccc(S(=O)(=O)N(C)[C@H](c2ccc(C(F)(F)F)cc2)C(F)(F)F)cn1]

Synthesis of (R)-6-methyl-N-(2,2,2-trifluoro-1-(4-(trifluoromethyl)phenyl)ethyl)pyridine-3-sulfonamide 167.1: Sulfonamide 167.1 was synthesized from 6-methylpyridine-3-sulfonyl chloride following the protocol as described in Method O using (R)-2,2,2-trifluoro-1-(4-(trifluoromethyl)phenyl)ethan-1-amine hydrochloride. The compound was purified by column chromatography over silica gel using 20% ethyl acetate in hexane to isolate desired compound 167.1 as yellow gum (200 mg, 48% yield). LCMS: m/z found 399.0 [M+H]⁺, rt=1.84 min (Method 3) [YMC Triart C18 column (3 μm, 33×2.1 mm)].

Synthesis (R)—N,6-dimethyl-N-(2,2,2-trifluoro-1-(4-(trifluoromethyl)phenyl)ethyl)pyridine-3-sulfonamide: The methylation of intermediate 167.1 was preformed following the protocol as described in Method D. The crude was purified by column chromatography over silica gel using 20% ethyl acetate in hexane to obtain compound Example 266 as a colourless sticky gum (50 mg, 99.61% purity, 47% yield). LCMS: m/z found 413.17 [M+H]⁺, rt=3.56 min (Method 2) [Waters Acquity BEH C8 column (1.7 μm, 50×2.1 mm)]. ¹H NMR (400 MHz, Chloroform-d) δ 8.95 (s, 1H), 7.99 (d, J=8.0 Hz, 1H), 7.69 (d, J=8.0 Hz, 2H), 7.59 (d, J=8.0 Hz, 2H), 7.32 (d, J=8.4 Hz, 1H), 5.94 (q, J=8.0 Hz, 1H), 2.70 (s, 3H), 2.66 (s, 3H).

### Example 267

### (R)—N-methyl-N-(2,2,2-trifluoro-1-(4-(trifluoromethyl)phenyl)ethyl)pyridine-3-sulfonamide

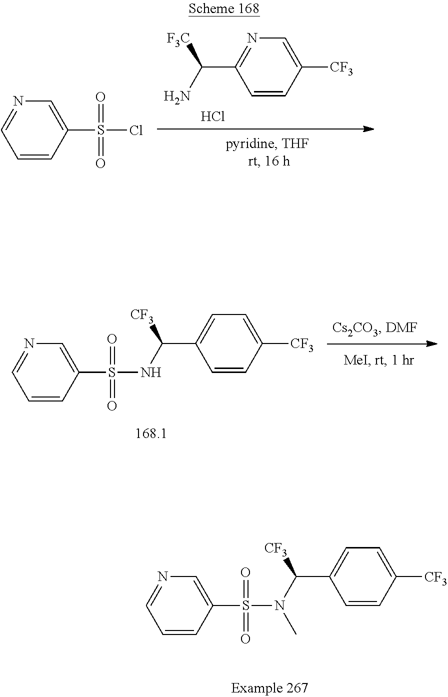 [SMILES: N[C@H](c1ccc(C(F)(F)F)cn1)C(F)(F)F ; O=S(=O)(Cl)c1cccnc1 ; Cl ; O=S(=O)(N[C@H](c1ccc(C(F)(F)F)cc1)C(F)(F)F)c1cccnc1 ; CN([C@H](c1ccc(C(F)(F)F)cc1)C(F)(F)F)S(=O)(=O)c1cccnc1]

Synthesis of (R)—N-(2,2,2-trifluoro-1-(4-(trifluoromethyl)phenyl)ethyl)pyridine-3-sulfonamide 168.1: Sulfonamide 168.1 was synthesized from pyridine-3-sulfonyl chloride following the protocol as described in Method O using (R)-2,2,2-trifluoro-1-(4-(trifluoromethyl)phenyl)ethan-1-amine hydrochloride. The compound was purified by column chromatography over silica gel using 20% ethyl acetate in hexane to isolate desired compound 168.1 as yellow gum (180 mg, 42% yield). LCMS: m/z found 385.0 [M+H]⁺, rt=1.84 min (Method 3) [YMC Triart C18 column (3 μm, 33×2.1 mm)].

Synthesis of (R)—N-methyl-N-(2,2,2-trifluoro-1-(4-(trifluoromethyl)phenyl)ethyl)pyridine-3-sulfonamide: The methylation of intermediate 168.1 was preformed following the protocol as described in Method D. The crude was purified by column chromatography over silica gel using 20% ethyl acetate in hexane to obtain compound Example 267 as light-yellow sticky gum (55 mg, 99.50% purity, 25% yield). ¹H NMR (400 MHz, Chloroform-d) δ 9.09 (s, 1H), 8.84 (d, J=4.0 Hz, 1H), 8.14 (d, J=7.7 Hz, 1H), 7.69 (d, J=8.3 Hz, 2H), 7.59 (d, J=8.1 Hz, 2H), 7.50 (t, J=4.8, 8.0 Hz, 1H), 5.94 (q, J=7.9 Hz, 1H), 2.73 (s, 3H).

### Example 268 and Example 269

### (R)-5-cyano-N-ethyl-N-(2,2,2-trifluoro-1-(3-methyl-4-(trifluoromethyl)phenyl)ethyl)pyridine-3-sulfonamide (Example 268) and (R)-5-cyano-N-methyl-N-(2,2,2-trifluoro-1-(3-methyl-4-(trifluoromethyl)phenyl)ethyl)pyridine-3-sulfonamide (Example 269)

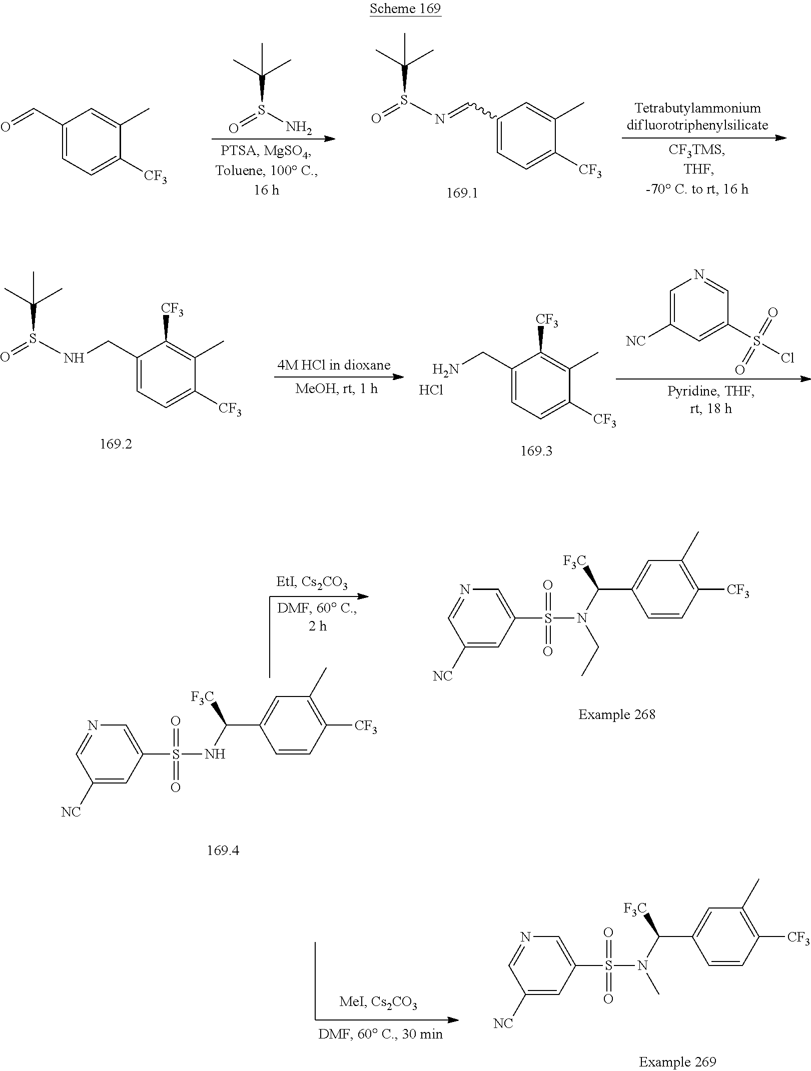

Synthesis of (S)-2-methyl-N-(3-methyl-4-(trifluoromethyl)benzylidene)propane-2-sulfinamide 169.1: Intermediate 169.1 was synthesized from 3-methyl-4-(trifluoromethyl)benzaldehyde using (S)-2-methylpropane-2-sulfinamide following the protocol as described in Method P-II. The crude compound was purified by column chromatography over silica gel using 10% ethyl acetate in hexane to isolate desired compound 169.1 as a yellow oil (1.20 g, 77% yield). ¹H NMR (400 MHz, DMSO-d₆): δ 8.60 (s, 1H), 8.00 (s, 1H), 7.94 (d, J=7.9 Hz, 1H), 7.83 (d, J=8.2 Hz, 1H), 1.20 (s, 9H).

Synthesis of (S)-2-methyl-N—((R)-2,2,2-trifluoro-1-(3-methyl-4-(trifluoromethyl)phenyl)ethyl)propane-2-sulfinamide 169.2: Intermediate 169.2 was synthesized from intermediate 169.1 following the protocol as described in Method Q-I. The crude compound was purified by column chromatography over silica gel using 10% ethyl acetate in hexane to isolate desired compound 169.2 as light-yellow gum (570 mg, 76% yield). LCMS: m/z found 362.1 [M+H]⁺, rt=1.97 min (Method 3) [YMC Triart C18 column (3 μm, 33×2.1 mm)].

Synthesis of (R)-2,2,2-trifluoro-1-(3-methyl-4-(trifluoromethyl)phenyl)ethan-1-amine 169.3: Intermediate 169.2 (600 mg) was dissolved in MeOH (2 mL) and cooled. 4M HCl in dioxane (2 mL) was added to it and the reaction mixture was stirred at room temperature for 1 h. The volatiles were evaporated under reduced pressure and the crude compound 169.3 was used in the forwarding step without further purification. LCMS: m/z found 258.0 [M+H]⁺, rt=1.71 min (Method 3) [YMC Triart C18 column (3 μm, 33×2.1 mm)].

Synthesis of (R)-5-cyano-N-(2,2,2-trifluoro-1-(3-methyl-4-(trifluoromethyl)phenyl)ethyl)pyridine-3-sulfonamide 169.4: Sulfonamide 169.4 was synthesized from 5-cyanopyridine-3-sulfonyl chloride following the protocol as described in Method O using (R)-2,2,2-trifluoro-1-(3-methyl-4-(trifluoromethyl)phenyl)ethan-1-amine hydrochloride 169.3. The compound was purified by column chromatography over silica gel using 20% ethyl acetate in hexane to isolate desired compound 169.4 as yellow gum (200 mg, 47%). LCMS: m/z found 424.0 [M+H]⁺, rt=1.92 min (Method 3) [YMC Triart C18 column (3 μm, 33×2.1 mm)].

Synthesis of (R)-5-cyano-N-ethyl-N-(2,2,2-trifluoro-1-(3-methyl-4-(trifluoromethyl)phenyl)ethyl)pyridine-3-sulfonamide: The ethylation of intermediate 169.4 was preformed following the protocol as described in Method D. The crude was purified by column chromatography over silica gel using 20% ethyl acetate in hexane to obtain compound Example 268 as a colorless sticky gum (30 mg, 96.90% purity, 44% yield). LCMS: m/z found 452.21[M+H]⁺, rt=3.37 min (Method 2) [Waters Acquity BEH C8 column (1.7 μm, 50×2.1 mm); ¹H NMR (400 MHz, Chloroform-d) δ 9.26 (d, J=1.9 Hz, 1H), 9.06 (d, J=2.0 Hz, 1H), 8.39 (t, J=2.1 Hz, 1H), 7.67 (d, J=8.0 Hz, 1H), 7.42 (d, J=10.1 Hz, 2H), 5.80 (q, J=8.3 Hz, 1H), 3.50-3.36 (m, 1H), 3.27-3.12 (m, 1H), 2.52 (d, J=2.0 Hz, 3H), 0.96 (t, J=7.0 Hz, 3H).

(R)-5-cyano-N-methyl-N-(2,2,2-trifluoro-1-(3-methyl-4-(trifluoromethyl)phenyl)ethyl)pyridine-3-sulfonamide: The methylation of intermediate 169.4 was performed following the protocol as described in Method D. The crude was purified by column chromatography over silica gel using 20% ethyl acetate in hexane to obtain compound Example 269 as a light-yellow sticky gum (70 mg, 99.40% purity, 45% yield). LCMS: m/z found 438.29 [M+H]⁺, rt=3.27 min (Method 2) [Waters Acquity BEH C8 column (1.7 μm, 50×2.1 mm)]; ¹H NMR (400 MHz, DMSO-d₆) δ 9.33 (s, 2H), 8.91 (s, 1H), 7.72 (d, J=8.3 Hz, 1H), 7.42 (d, J=8.1 Hz, 1H), 7.34 (s, 1H), 6.13 (q, J=8.2 Hz, 1H), 2.89 (s, 3H), 2.40 (s, 3H).

### Example 270 and Example 271

### (R)-5-fluoro-N-methyl-N-(2,2,2-trifluoro-1-(3-methyl-4-(trifluoromethyl)phenyl)ethyl)pyridine-3-sulfonamide (Example 270) and (R)—N-ethyl-5-fluoro-N-(2,2,2-trifluoro-1-(3-methyl-4-(trifluoromethyl)phenyl)ethyl)pyridine-3-sulfonamide (Example 271)

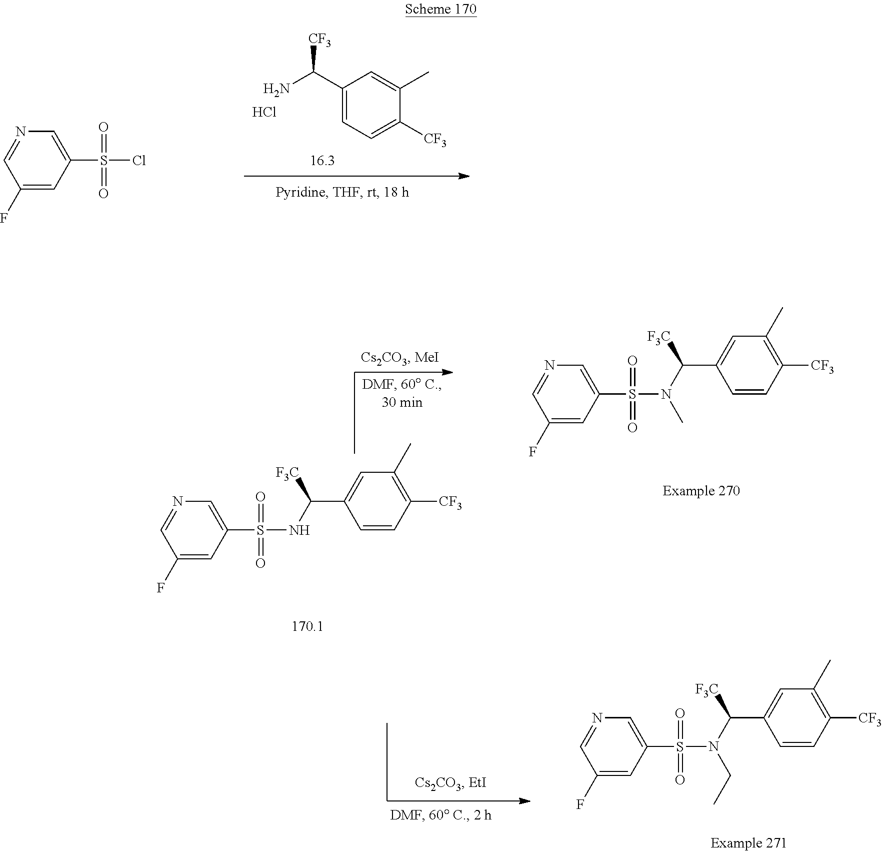 [SMILES: O=S(=O)(Cl)c1cncc(F)c1 ; Cl ; Cc1cc([C@@H](N)C(F)(F)F)ccc1C(F)(F)F ; Cc1cc([C@@H](NS(=O)(=O)c2cncc(F)c2)C(F)(F)F)ccc1C(F)(F)F ; Cc1cc([C@@H](N(C)S(=O)(=O)c2cncc(F)c2)C(F)(F)F)ccc1C(F)(F)F ; CCN([C@H](c1ccc(C(F)(F)F)c(C)c1)C(F)(F)F)S(=O)(=O)c1cncc(F)c1]

Synthesis of (R)-5-fluoro-N-(2,2,2-trifluoro-1-(3-methyl-4-(trifluoromethyl)phenyl)ethyl)pyridine-3-sulfonamide 170.1: Sulfonamide 170.1 was synthesized from 5-fluoropyridine-3-sulfonyl chloride following the protocol as described in Method O using (R)-2,2,2-trifluoro-1-(3-methyl-4-(trifluoromethyl)phenyl)ethan-1-amine hydrochloride 16.3. The compound was purified by column chromatography over silica gel using 20% ethyl acetate in hexane to isolate desired compound 170.1 as a yellow oil (420 mg, 63%). LCMS: m/z found 417.0 [M+H]⁺, rt=1.94 min (Method 3) [YMC Triart C18 column (3 μm, 33×2.1 mm)].

Synthesis of (R)-5-fluoro-N-methyl-N-(2,2,2-trifluoro-1-(3-methyl-4-(trifluoromethyl)phenyl)ethyl)pyridine-3-sulfonamide: The methylation of intermediate 170.1 was preformed following the protocol as described in Method D. The crude was purified by column chromatography over silica gel using 20% ethyl acetate in hexane to obtain compound Example 270 as a colourless sticky gum (80 mg, 99.85% purity, 30% yield). LCMS: m/z found 431.17 [M+H]⁺, rt=3.33 min (Method 2) [Waters Acquity BEH C8 column (1.7 μm, 50×2.1 mm)]; ¹H NMR (400 MHz, Chloroform-d): δ 8.90 (s, 1H), 8.71 (d, J=2.7 Hz, 1H), 7.87-7.79 (m, 1H), 7.66 (d, J=8.1 Hz, 1H), 7.40-7.31 (m, 2H), 5.86 (q, J=8.1 Hz, 1H), 2.76 (s, 3H), 2.51 (s, 3H).

Synthesis of (R)—N-ethyl-5-fluoro-N-(2,2,2-trifluoro-1-(3-methyl-4-(trifluoromethyl)phenyl)ethyl)pyridine-3-sulfonamide: The ethylation of intermediate 17.1 was preformed following the protocol as described in Method D. The crude was purified by column chromatography over silica gel using 20% ethyl acetate in hexane to obtain compound Example 271 as colourless sticky gum (30 mg, 96.09% purity, 20% yield). LCMS: m/z found 445.30 [M+H]⁺, rt=3.37 min (Method 2) [Waters Acquity BEH C8 column (1.7 μm, 50×2.1 mm)]; ¹H NMR (400 MHz, DMSO-d₆) δ 8.99 (s, 1H), 8.93 (d, J=2.6 Hz, 1H), 8.38-8.30 (m, 1H), 7.72 (d, J=8.2 Hz, 1H), 7.44 (d, J=8.2 Hz, 1H), 7.31 (s, 1H), 6.14 (q, J=8.4 Hz, 1H), 3.56-3.42 (m, 1H), 3.31-3.24 (m, 1H), 2.37 (d, J=1.9 Hz, 3H), 1.03 (t, J=7.0 Hz, 3H).

### Example 272

### 5-Cyano-N-methyl-N-(2,2,2-trifluoro-1-(4-fluorophenyl)ethyl)pyridine-3-sulfonamide

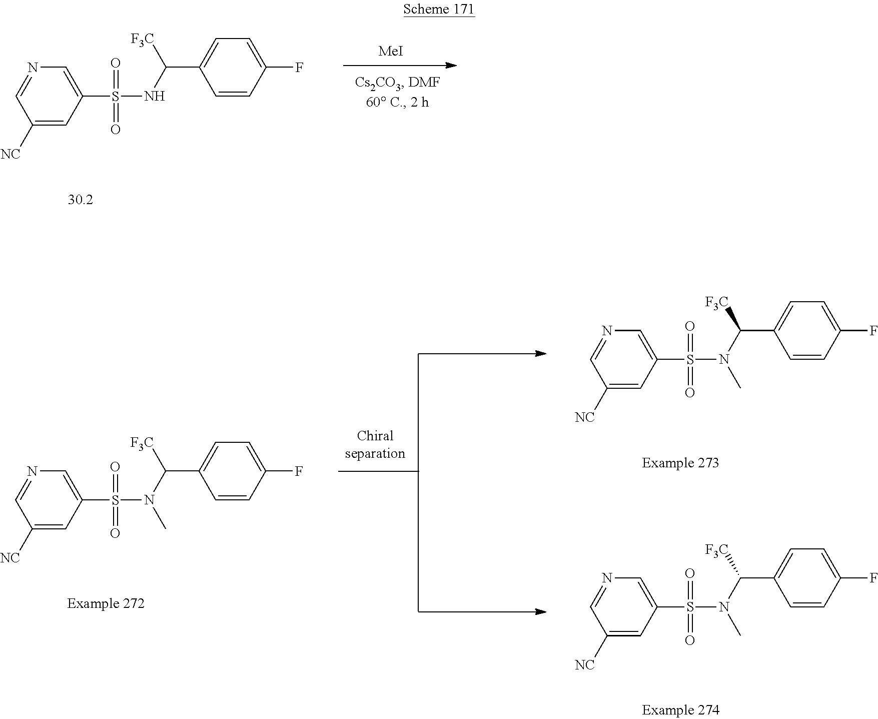 [SMILES: "MeI" ; N#Cc1cncc(S(=O)(=O)NC(c2ccc(F)cc2)C(F)(F)F)c1 ; CN([C@H](c1ccc(F)cc1)C(F)(F)F)S(=O)(=O)c1cncc(C#N)c1 ; CN(C(c1ccc(F)cc1)C(F)(F)F)S(=O)(=O)c1cncc(C#N)c1 ; CN([C@@H](c1ccc(F)cc1)C(F)(F)F)S(=O)(=O)c1cncc(C#N)c1]

5-Cyano-N-(2,2,2-trifluoro-1-(4-fluorophenyl)ethyl)pyridine-3-sulfonamide 30.2: Intermediate 30.2 was synthesized from 5-cyanopyridine-3-sulfonyl chloride following the protocol as described in Method O using 2,2,2-trifluoro-1-(4-fluorophenyl)ethan-1-amine hydrochloride at 60° C. The compound was purified by column chromatography over silica gel using 35% ethyl acetate in hexane and isolated as yellow sticky solid (600 mg, 82% yield). LCMS: m/z found 373.9 [M+H]⁺, rt=3.47 min (Method 4) [Waters Xbridge C8 column (5 μm, 50×4.6 mm)]; ¹H NMR (400 MHz, DMSO-d₆) δ 9.80 (s, 1H), 9.14 (d, J=1.5 Hz, 1H), 9.00 (d, J=2.0 Hz, 1H), 8.44 (t, J=2.1 Hz, 1H), 7.44 (dd, J=5.3, 8.6 Hz, 2H), 7.11 (t, J=8.8 Hz, 2H), 5.58-5.42 (m, 1H).

5-Cyano-N-methyl-N-(2,2,2-trifluoro-1-(4-fluorophenyl)ethyl)pyridine-3-sulfonamide: The methylation of intermediate 30.2 was performed following the protocol as described in Method D at 60° C. The crude was purified by column chromatography over silica gel using 30% ethyl acetate in hexane and the desired compound Example 272 was isolated as a light-yellow sticky gum (100 mg, 48% yield, 99.64% purity). ¹H NMR (400 MHz, DMSO-d₆) δ 9.31 (d, J=1.7 Hz, 2H), 8.88 (t, J=2.1 Hz, 1H), 7.44 (dd, J=5.3, 8.6 Hz, 2H), 7.24 (t, J=8.8 Hz, 2H), 6.08 (q, J=8.4 Hz, 1H), 2.86 (s, 3H).

### Example 273 and Example 274

### (S)-5-Cyano-N-methyl-N-(2,2,2-trifluoro-1-(4-fluorophenyl)ethyl)pyridine-3-sulfonamide (Example 273) and (R)-5-Cyano-N-methyl-N-(2,2,2-trifluoro-1-(4-fluorophenyl)ethyl)pyridine-3-sulfonamide (Example 274)

Chiral separation of racemic Example 272 provided both enantiomers as described below. Chiral separation method: Chiral separation was done on Agilent 1200 series instrument. Column: CHIRALPAK IA (250×20 mm), 5μ, operating at ambient temperature and at a flow rate of 18.0 mL/min. Mobile phase was a mixture of 75% hexane and 25% of ethanol, holding this isocratic mixture run up to 32 min at a wavelength of 224 nm.

Example 273: Colorless Sticky Gum (12 mg, 99.89% purity). LCMS: m/z found 372.0 [M−H], rt=2.80 min (Method 9) [Waters Xbridge C18 column (3.5 μm, 50×3 mm)]; ¹H NMR (400 MHz, DMSO-d₆) δ 9.31 (s, 2H), 8.88 (s, 1H), 7.43 (t, J=6.1 Hz, 2H), 7.24 (t, J=8.5 Hz, 2H), 6.08 (q, J=8.3 Hz, 1H), 2.86 (s, 3H).

Example 274: Light yellow Sticky Gum (10 mg, 99.80% purity). LCMS: m/z found 372.0 [M−H], rt=2.80 min (Method 9) [Waters Xbridge C18 column (3.5 μm, 50×3 mm)]; ¹H NMR (400 MHz, DMSO-d₆) δ 9.31 (s, 2H), 8.88 (s, 1H), 7.43 (t, J=5.6 Hz, 2H), 7.24 (t, J=8.6 Hz, 2H), 6.08 (q, J=8.3 Hz, 1H), 2.86 (s, 3H).

### Pharmacological Examples

### Example 275: Human Cav2.3 Channel Calcium-Influx Assay

### Cell Line

HEK-293 T-Rex cells were transfected with pcDNA3.1-Kir2.1 to generate a stable cell line. After antibiotic selection, the obtained stable pools were analysed using FLIPR^{TETRA} (membrane potential assay), and a limiting dilution was performed and a HEK-293 T-Rex/Kir2.1 clone was selected. The clone was transfected with pcDNA3.1-Cav2.3e and pBud-Cav-β4-α2δ1 to generate a stable cell line where the Cav2.3e expression is inducible. After antibiotic selection, functional clone pool analysis and two successive limiting dilutions, the final clone underwent a qPCR analysis and a biophysical and pharmacological validation using patch-clamp.

### Assay

A compound plate, containing 10 test compounds in eight-points dose response with n=4, was prepared. Test compounds dose response curves were prepared in automated fashion in 100% DMSO at CyBio Felix and starting from a stock solution in 100% DMSO; a defined volume was serially moved into destination wells pre-filled with the desired DMSO volume; stock solution concentration volumes were dependent on the final compounds concentrations to be tested and the final DMSO content used in the assay. For the assay test compounds dose response curves ranged from 30 μM and half log dilution with 0.3% final DMSO. In order to obtain the 8-points dose response curve all the concentrations were created by starting from a 10 mM DMSO stock solution and then moving 9.5 μL into a destination well pre-filled with 20.5 μL of 100% DMSO and repeating this step seven times. This initial step was done into 96 MTP plates, 10 compounds/plate. One 384 MTP was then reformatted to contain all the 10 compounds at 8 concentrations, quadruplicate data points. This so obtained 384 MTP compound plate served as source plate in a “mother to child” process with a CyBi®-Well dispenser in which 0.7 μL of compounds were moved into a destination plate pre-filled with 57.6 μL of Tyrode's buffer 0 mM K+, thus obtaining 4× concentrated compounds solution. In columns 1-2 and 23-24 the control wells were added. Both “source” compound plate and “destination” compound plate were barcoded and a relationship between the two plates was thus generated.

The day before the experiment, cells were detached by gentle wash with DPBS, followed by 5 min incubation at 37° C. with Trypsin solution. Detached cells were diluted with OptiMEM+Doxycycline at 0.2 μg/mL, counted and plated in Poly-D-Lysine coated black/clear bottom (15.000 c/well in 20 μl/well) by the use of a MATRIX WellMate dispenser. Plates were placed into a humidified incubator at 37° C. with 5% CO₂ until the experimental day. 24 h after seeding 10 μL/well of 1.5× Fluo8 NW dye solution prepared in Tyrode's buffer 0 mM K* were added on top of the seeding medium. Cell plates were incubated for 40-60 min at RT in the dark. We then Injected off-line 10 μL/well of test compounds and controls 4× concentrated in Tyrode's Buffer 0 mM K+ with the CyBi®-Well instrument. Cell plates were incubated 3 minutes at RT. Finally, we injected 20 μL/well of 3× concentrated activator solution (K20-Na130-Ca2 buffer: 20 mM KCl, 130 mM NaCl, 2 mM CaCl2, 10 mM HEPES, 10 mM Glucose, final concentrations, prepared starting from “K0-Na150-Ca2” and “K150-Na0-Ca2” buffers) at FDSS7000EX instrument and read emitted fluorescence for 130 seconds.

Data analysis was performed with Genedata Screener® software and reported compounds activity as % effect in relation to the normalization standards. The Kinetic Response Value (KRV) is calculated as follows:

KRV=Maximal fluorescence recorded from second 5 to second 130 minus baseline fluorescence, computed as average from second 1 to second 2, of the kinetic trace. The KRV was normalized versus Neutral Controls and Inhibitor Controls in order to obtain the Activity[%] for each well. The normalization places the compound activity values on an equivalent scale and makes them comparable across plates or different compound batches. Therefore, the compound activity values were scaled (based on the two references) to a common range (two-point normalization). The following equation was used by the software to normalize the signal values to the desired signal range:

$$N ( x ) = CR + [ ( ( x - 〈 cr 〉 ) / ( 〈 sr 〉 - 〈 cr 〉 ) ) \cdot ( S R - CR ) ]$$

where: x is the calculated signal value of a well (KRV); <cr> is the median of the calculated signal values (KRV) for the Central Reference wells of a plate (median of Neutral Controls); <sr> is the median of the calculated signal values (KRV) for the Scale Reference wells of a plate (median of Inhibitor Controls); CR is the desired median normalized value for the Central Reference (0) and SR is the desired median normalized value for the Scale Reference (−100).

The final equation to calculate the Activity % can be simplified as follow:

$$\% Activity = - 100 \cdot ( x - 〈 NeutralControls 〉 ) / ( 〈 NeutralControls 〉 - 〈 InhibitorControls 〉 )$$

$$where full inhibition corresponds to \% Activity = - 100$$

The fitting of the dose-response curve of each test compound is performed in the Analyzer module of the Screener software on the normalized values and applying the “smart fit” strategy. This strategy allowed an automatic selection between the “Constant Fit” and the “Hill Fit” model calculating which fit model best matched the experimental data. The Constant Fit was applied when no change of activity was detected across the measured concentrations, and the corresponding compounds were further classified as inactive. The Hill Fit was applied when the observed activity significantly changed with the compound concentration. In case of Hill Fit, Hill equation was used to determine the concentration at which activity reaches 50% of maximum level, i.e., AC₅₀.

$$Y = {S}_{0} + ( ( {S}_{inf} - {S}_{0} ) / ( 1 + {( 1 {0}^{L o g A C 50} / {10}^{X} )}^{n} ) )$$

where X is Log10 of compound concentration. The equation has four parameters:
Zero Activity (S0)—Activity level at zero concentration of test compound; Infinite Activity (S_{inf})—Activity level at infinite concentration of test compound; AC₅₀—Concentration at which activity reaches 50% of maximum level. This term corresponds to IC₅₀ in this assay; AND Hill coefficient (n)—Measure of the slope at AC₅₀.

The pIC₅₀ values measured in this assay for the exemplified compounds is set out in the table below:

| | Cav2.3 |
|---|---|
| Example | (pIC₅₀) |
| 1 | 5.05 |
| 2 | 6.14 |
| 3 | 6.31 |
| 4 | 5.30 |
| 5 | 6.56 |
| 6 | 6.22 |
| 7 | 5.44 |
| 8 | 5.53 |
| 9 | 5.16 |
| 10 | 5.66 |
| 11 | 5.22 |
| 12 | 5.00 |
| 13 | 5.41 |
| 14 | 5.64 |
| 15 | 4.94 |
| 16 | 5.78 |
| 17 | 5.44 |
| 18 | 4.93 |
| 19 | 5.62 |
| 20 | 6.10 |
| 21 | 6.36 |
| 22 | 6.13 |
| 23 | 5.15 |
| 24 | 4.89 |
| 25 | 6.55 |
| 26 | 7.49 |
| 27 | 6.29 |
| 28 | 6.45 |
| 29 | 6.18 |
| 30 | 6.62 |
| 31 | 6.43 |
| 32 | 5.42 |
| 33 | 6.30 |
| 34 | 6.20 |
| 35 | 7.87 |
| 36 | 6.58 |
| 37 | 4.87 |
| 38 | 6.91 |
| 39 | 5.82 |
| 40 | 5.62 |
| 41 | 4.72 |
| 42 | 6.90 |
| 43 | 6.69 |
| 44 | 5.23 |
| 45 | 6.07 |
| 46 | 6.33 |
| 47 | 6.30 |
| 48 | 5.70 |
| 49 | 6.47 |
| 50 | 6.62 |
| 51 | 6.43 |
| 52 | 7.02 |
| 53 | 5.75 |
| 54 | 7.43 |
| 55 | 6.33 |
| 56 | 6.92 |
| 57 | 6.96 |
| 58 | 6.35 |
| 59 | 5.21 |
| 60 | 7.14 |
| 61 | 7.01 |
| 62 | 5.20 |
| 63 | 6.92 |
| 64 | 6.75 |
| 65 | 5.42 |
| 66 | 7.39 |
| 67 | 6.97 |
| 68 | 7.45 |
| 69 | 5.25 |
| 70 | 6.72 |
| 71 | 6.80 |
| 72 | 6.20 |
| 73 | 5.50 |
| 74 | 5.83 |
| 75 | 5.94 |
| 76 | 5.31 |
| 77 | 7.01 |
| 78 | 5.69 |
| 79 | 4.99 |
| 80 | 6.35 |
| 81 | 6.02 |
| 82 | 6.04 |
| 83 | 5.42 |
| 84 | 6.06 |
| 85 | 6.34 |
| 86 | 5.95 |
| 87 | 5.50 |
| 88 | 5.60 |
| 89 | 5.68 |
| 90 | 6.20 |
| 91 | 5.37 |
| 92 | 5.88 |
| 93 | 6.53 |
| 94 | 5.85 |
| 95 | 5.62 |
| 96 | 6.95 |
| 97 | 5.22 |
| 98 | 5.80 |
| 99 | 6.56 |
| 100 | 6.42 |
| 101 | 6.74 |
| 102 | 5.17 |
| 103 | 6.50 |
| 104 | 7.24 |
| 105 | 5.38 |
| 106 | 7.51 |
| 107 | 5.08 |
| 108 | 4.30 |
| 109 | 4.59 |
| 110 | 7.11 |
| 111 | 7.63 |
| 112 | 5.95 |
| 113 | 6.96 |
| 114 | 6.87 |
| 115 | 5.44 |
| 116 | 5.81 |
| 117 | 5.38 |
| 118 | 5.25 |
| 119 | 7.06 |
| 120 | 6.88 |
| 121 | 6.72 |
| 122 | 5.14 |
| 123 | 6.78 |
| 124 | 6.11 |
| 125 | 5.37 |
| 126 | 4.52 |
| 127 | 6.51 |
| 128 | 4.85 |
| 129 | 5.93 |
| 131 | 5.12 |
| 132 | 6.05 |
| 133 | 7.20 |
| 134 | 7.65 |
| 135 | 6.76 |
| 136 | 7.50 |
| 137 | 7.14 |
| 138 | 6.78 |
| 139 | 6.31 |
| 140 | 6.25 |
| 141 | 6.24 |
| 142 | 5.97 |
| 143 | 5.38 |
| 144 | 5.75 |
| 145 | 7.60 |
| 146 | 7.76 |
| 147 | 6.80 |
| 148 | 6.16 |
| 149 | 6.61 |
| 150 | 7.40 |
| 151 | 6.77 |
| 152 | 6.51 |
| 153 | 5.80 |
| 154 | 6.70 |
| 155 | 5.72 |
| 156 | 6.69 |
| 157 | 5.52 |
| 158 | 6.56 |
| 159 | 6.33 |
| 160 | 6.06 |
| 161 | 6.22 |
| 162 | 6.01 |
| 163 | 5.76 |
| 164 | 6.76 |
| 165 | 6.91 |
| 166 | 6.10 |
| 167 | 6.48 |
| 168 | 5.66 |
| 169 | 5.24 |
| 170 | 4.88 |
| 171 | 7.59 |
| 172 | 7.23 |
| 173 | 4.95 |
| 174 | 4.73 |
| 175 | 6.52 |
| 176 | 7.04 |
| 177 | 7.47 |
| 178 | 6.75 |
| 179 | 6.18 |
| 180 | 6.35 |
| 181 | 6.27 |
| 182 | 6.54 |
| 183 | 6.62 |
| 184 | 6.35 |
| 185 | 6.81 |
| 186 | 7.42 |
| 187 | 7.72 |
| 188 | 6.51 |
| 189 | 6.78 |
| 190 | 6.00 |
| 191 | 6.84 |
| 192 | 6.18 |
| 193 | 6.65 |
| 194 | 6.62 |
| 195 | 6.99 |
| 196 | 5.03 |
| 197 | 5.21 |
| 198 | 6.49 |
| 199 | 5.94 |
| 200 | 6.55 |
| 201 | 6.37 |
| 202 | 6.54 |
| 203 | 6.10 |
| 204 | 6.12 |
| 205 | 5.89 |
| 206 | 6.60 |
| 207 | 6.02 |
| 208 | 5.98 |
| 209 | 6.72 |
| 210 | 6.85 |
| 211 | 7.17 |
| 212 | 6.80 |
| 213 | 6.07 |
| 214 | 6.95 |
| 215 | 7.15 |
| 216 | 7.29 |
| 217 | 6.85 |
| 218 | 5.45 |
| 219 | 7.93 |
| 220 | 7.52 |
| 221 | 6.63 |
| 222 | 6.61 |
| 223 | 6.84 |
| 224 | 6.85 |
| 225 | 6.98 |
| 226 | 6.79 |
| 227 | 7.55 |
| 228 | 6.05 |
| 229 | 6.93 |
| 230 | 7.00 |
| 231 | 5.56 |
| 232 | 6.04 |
| 233 | 5.91 |
| 234 | 6.38 |
| 235 | 7.50 |
| 236 | 7.33 |
| 237 | 7.52 |
| 238 | 7.44 |
| 239 | 7.30 |
| 240 | 6.79 |
| 241 | 6.98 |
| 242 | 7.45 |
| 243 | 5.19 |
| 244 | 5.84 |
| 245 | 5.70 |
| 246 | 7.31 |
| 247 | 5.11 |
| 248 | 5.14 |
| 249 | 5.73 |
| 250 | 7.14 |
| 251 | 7.18 |
| 252 | 5.71 |
| 253 | 5.56 |
| 254 | 7.33 |
| 255 | 6.96 |
| 256 | 5.37 |
| 257 | 7.64 |
| 258 | 5.87 |
| 259 | 6.61 |
| 260 | 5.45 |
| 261 | 7.93 |
| 262 | 7.52 |
| 263 | 6.63 |
| 264 | 4.96 |
| 265 | 7.40 |
| 266 | 6.54 |
| 267 | 7.09 |
| 268 | 6.33 |
| 269 | 6.52 |
| 270 | 6.31 |
| 271 | 6.79 |
| 272 | 6.81 |
| 273 | 5.83 |
| 274 | 7.00 |

“NT”: not tested

The inventors have found that for some high affinity compounds, the addition of pluronic acid (0.05%) aided solubility and in doing so increased the measured pIC₅₀ values by approximately 0.5 units. However, all of the reported pIC₅₀ values presented above did not include pluronic acid.

### Example 276: Whole-Cell Patch Clamp Assay

HEK-293 T-Rex/Kir2.1/Cav2.3e-β4-α2δ1 cells were seeded 96 hours before experiment and doxycycline at 0.2 μg/mL was added 24 hours before experiment. Just before the experiments cells were washed twice with D-PBS w/o Ca²⁺/Mg²⁺ and gently detached from the flask with Detachin. Cells were then re-suspended in the suspension solution (25 mL EX-CELL ACF CHO medium; 0.25 mL of 100× Penicillin/Streptomycin) and placed on the Automated Patch-clamp platform (QPatch 16X).

Standard whole-cell voltage clamp experiments were performed at room temperature using the single hole technology and the following intracellular and extracellular solutions (ICS and ECS, respectively) were used. ICS (mM): CsF 60, CsCl 50, NaCl 10, EGTA 20, BAPTA 5, HEPES 10, NaGTP 0.3, MgATP 5 (pH 7.2; ECS (mM): NMDG-Cl 120, BaCl2 20, HEPES 10, EGTA 7 (pH 7.4). After establishment of the seal and the passage in the whole-cell configuration, the Cav2.3 dependent current was evoked by challenging the cells with the following voltage protocol: the plasma membrane was held at −80 mV then a 50 ms-long depolarizing pulse at 0 mV, followed by a 50 ms-long hyperpolarizing pulse at −100 mV were applied and finally the plasma membrane was held back to −80 mV. This voltage protocol was applied every 10 seconds. Cav2.3 dependent current was first measured in control condition (vehicle, 0.1% DMSO), in the presence of increasing concentration of the compound under investigation and finally SNX 482 was added to fully block any R-type Cav2.3 dependent current.

For data collection, the Sophion proprietary software was used while the analysis was performed off-line using Excel and GraphPad Prism. The inward current (Area Under Curve) measured during each depolarizing pulse was normalized to cell membrane capacitance (Cm) to obtain the average inward current density in each condition (control, compound under investigation and SNX 482). Then, the inhibitory effect of the compound under investigation was evaluated as % of the remaining inward current, normalized to the internal control. When the percentage of inhibition exerted by the highest concentration tested was superior to 50%, the dose-response curves data was fitted with the following equation: Y=100/(1+10\hat{ }((LogIC50−X)*Hillslope)); where X: log of concentration; Y: normalized response, 100% down to 0%, decreasing as X increases; LogIC50: same log units as X; Hillslope: slope factor or hill slope, unit less. All data were expressed as mean±S.E

The pIC₅₀ values for some of the exemplified compounds of the invention are set out in the table below:

| | Cav2.3 |
|---|---|
| Example | (pIC₅₀) |
| 2 | 5.83 ± 0.14 |
| 4 | 6.35 ± 0.05 |
| 5 | 5.87 ± 0.05 |
| 9 | 5.72 ± 0.11 |
| 13 | 5.59 ± 0.07 |
| 20 | 6.15 ± 0.16 |
| 35 | 7.33 ± 0.05 |
| 63 | 6.52 ± 0.07 |
| 68 | 7.14 ± 0.04 |
| 77 | 6.25 ± 0.13 |
| 96 | 7.17 ± 0.11 |
| 133 | 6.83 ± 0.08 |
| 136 | 7.19 ± 0.04 |
| 171 | 6.88 ± 0.06 |
| 172 | 6.53 ± 0.07 |
| 178 | 6.42 ± 0.08 |
| 191 | 6.34 ± 0.10 |
| 219 | 7.37 ± 0.04 |
| 220 | 6.85 ± 0.57 |

### Example 277: Ex Vivo Activity in Substantia Nigra Dopamine Neurons in Mouse Brain Slice Experiments

Compounds of the invention were tested on R-type calcium current with whole cell patch clamp electrophysiology in substantia nigra dopamine neurons in an ex vivo brain slice preparation according to the methods described in Siller et al., Elife, 11:e67464 (2022) https://doi.org/i0.7554/eLife.67464.

Example 35 was found active in this model (blocking R-type calcium current) at concentrations of 10 μM

### Example 278: In Vivo Activity in the Maximal Electroshock Stimulation Model (MES) in Mouse

Compounds of the invention were tested in an in vivo model of epilepsy, the MES model in mouse, according to the method described in Kehne et al, Neurochemistry Research 42:1894-1903 (2017) 1 https://doi.org/10.1007/s11064-017-2275-z.

Example 35 of the invention was found active in this model at the dose of 1.5 mg/kg after oral administration.

1. A compound of the formula (I), or a pharmaceutically acceptable salt thereof: 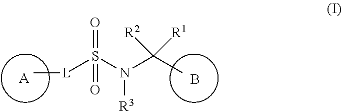
wherein:
R¹ is selected from: C₁₋₆ alkyl, C₃₋₆ cycloalkyl, and C₃₋₆ cycloalkyl-C₁₋₆ alkyl-, wherein R¹ is substituted by at least one fluorine; optionally wherein one or more H in R¹ is substituted by D;
R² is selected from: H, D, C₁₋₆ alkyl and C₁₋₆ haloalkyl; or
R¹ and R² together with the carbon atom to which they are attached form a C3.6 cycloalkyl substituted with at least one fluorine;
R³ is selected from: C₁₋₆ alkyl and C₁₋₆ haloalkyl; optionally wherein one or more H in R³ is substituted by D;
L is selected from: a bond and C₁₋₃ alkylene;
Ring A is selected from: C₃₋₆ cycloalkyl, 4- to 7-membered heterocyclyl, 5- to 12-membered heteroaryl and C₆₋₁₀ aryl; wherein Ring A is optionally substituted by one or more R⁴;
each R⁴ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
wherein said C₁₋₆ alkyl, 2 to 8 membered heteroalkyl, C₂₋₆ alkenyl and C₂₋₆ alkynyl is optionally substituted by one or more R⁷;
R⁵ and R⁶ are each independently selected from: H, C₁₋₆ alkyl, C₁₋₆ haloalkyl and Q¹,
wherein said C₁₋₆ alkyl is optionally substituted by one or more R⁸;
each R⁷ and R⁸ is independently selected from: halo, —CN, —OR^{7A}, —S(O)ₓR^{7A}, —NR^{7A}R^{7B}, C(O)R^{7A}, —OC(O)R^{7A}, —C(O)OR^{7A}, —NR^{7A}C(O)R^{7B}, —C(O)NR^{7A}R^{7B} and Q²;
each Q¹ and Q² is independently selected from: C₃₋₆ cycloalkyl, 4- to 7-membered heterocyclyl, phenyl and 5- or 6-membered heteroaryl,
wherein said C₃₋₆ cycloalkyl, 4- to 7-membered heterocyclyl, phenyl and 5- or 6-membered heteroaryl is optionally substituted by one or more R⁹;
each R⁹ is independently selected from: halo, —O, —CN, —NO₂, C₁₋₄ alkyl, C₁₋₄ haloalkyl, —OR^{9A}, —S(O)₂R^{9A}, —NR^{9A}R^{9B}, —C(O)R^{9A}, —OC(O)R^{9A}, —C(O)OR^{9A}, —NR^{9B}C(O)R^{9A}, —C(O)NR^{9A}R^{9B}, —NR^{9B}C(O)OR^{9A}, —OC(O)NR^{9A}R^{9B}, —NR^{9B}SO₂R^{9A} and —SO₂NR^{9A}R^{9B},
wherein said C₁₋₄ alkyl is optionally substituted by 1 or 2 substituents selected from: halo, —CN, —OR^{9C}, —NR^{9C}R^{9D} and —SO₂R^{9C};
Ring B is phenyl or a 5- or 6-membered heteroaryl, wherein Ring B is optionally substituted by one or more R¹⁰;
each R¹⁰ is independently selected from: halo, —CN, —NO₂, ═O, C₁₋₆ alkyl, C₁₋₆ haloalkyl, 2 to 8 membered heteroalkyl, C₂₋₆ alkenyl, C₂₋₆ alkynyl, —OR^{10A}, —S(O)ₓR^{10A}, —NR^{10A}R^{10B}, —C(O)R^{10A}, —OC(O)R^{10A}, —C(O)OR^{10A}, —NR^{10A}C(O)R^{10B}, —C(O)NR^{10A}R^{10B}, —NR^{10A}C(O)OR^{10B}, —OC(O)NR^{10A}R^{10B}, —NR^{10A}SO₂R^{10B}, and —SO₂NR^{10A}R^{10B},
wherein said C₁₋₆ alkyl, 2 to 8 membered heteroalkyl, C₂₋₆ alkenyl and C₂₋₆ alkynyl is optionally substituted by one or more R¹¹;
each R¹¹ is independently selected from: halo, —CN, —OR^{11A}, —NR^{11A}R^{11B} and —SO₂R^{11A},
R^{7A}, R^{7B}, R^{9A}, R^{9B}, R^{9C}, R^{9D}, R^{10A}, R^{10B}, R^{11A} and R^{11B} are at each occurrence independently selected from: H, C₁₋₄ alkyl and C₁₋₄ haloalkyl;
and wherein any —NR⁵R⁶, —NR^{7A}R^{7B}, —NR^{9A}R^{9B}, —NR^{9C}R^{9D}, —NR^{10A}R^{10B}, and —NR^{11A}R^{11B} within a substituent may form a 4- to 6-membered heterocyclyl,
wherein said 4- to 6-membered heterocyclyl is optionally substituted by one or more substituents selected from: halo, ═O, C₁₋₄ alkyl and C₁₋₄ haloalkyl;
each x is independently 0, 1, or 2;
with the following provisos:
(i) that when the group 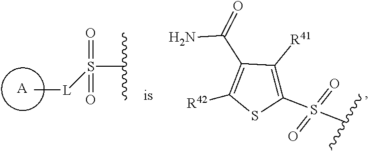
wherein R⁴¹ is H, —CH₃, —CF₃ or cyclopropyl, then R⁴² is not —NHC(O)R⁶; and
(ii) that Compounds A and B are excluded:

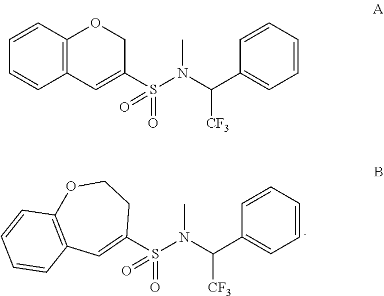 [SMILES: CN(C(c1ccccc1)C(F)(F)F)S(=O)(=O)C1=Cc2ccccc2OC1 ; CN(C(c1ccccc1)C(F)(F)F)S(=O)(=O)C1=Cc2ccccc2OCC1]

2. The compound according to claim 1, wherein Ring A is a monocyclic 6-membered heteroaryl or a 9-membered fused bicyclic heteroaryl, wherein Ring A has 1 to 4 ring nitrogen atoms and wherein Ring A is optionally substituted with one or more R⁴.

3. The compound according to claim 1, wherein Ring A is a monocyclic 6-membered heteroaryl, wherein Ring A has 1, 2 or 3 ring nitrogen atoms and wherein Ring A is optionally substituted with one or more R⁴.

4. The compound according to claim 1, wherein Ring A is selected from: thienyl, thiazolyl, pyridyl, pyrimidyl, pyrazinyl, pyridazinyl, tetrahydropyranyl, 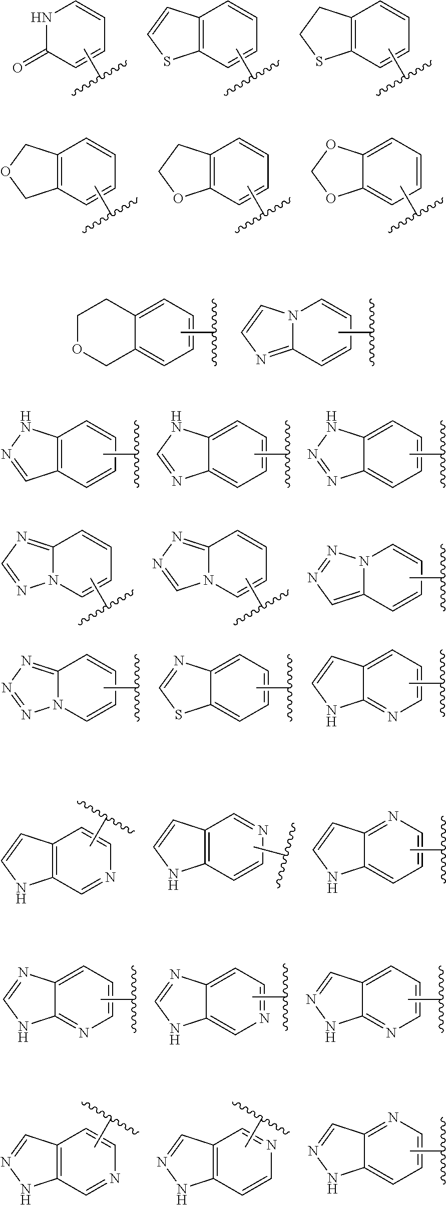 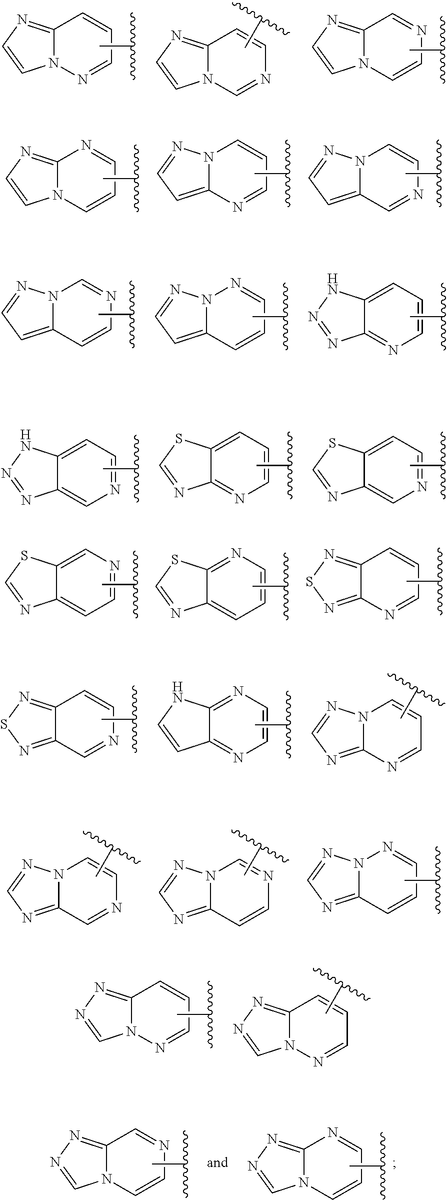
wherein Ring A is optionally substituted with one or more R⁴.

5. The compound according to claim 1, wherein Ring A is selected from: 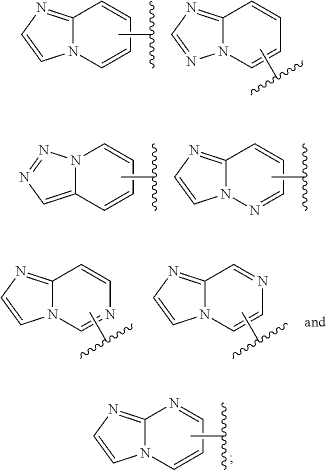
wherein Ring A is optionally substituted with one or more R⁴.

6. The compound according to claim 1, wherein Ring A is selected from: 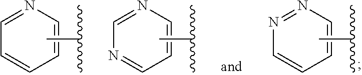
wherein Ring A is optionally substituted with one or more R⁴.

7. The compound according to claim 1, wherein Ring A is selected from:

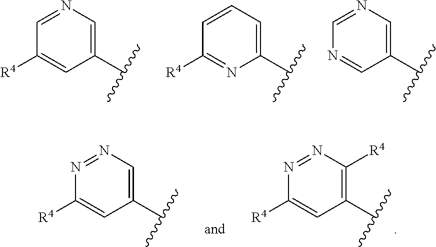

8. The compound according to claim 1, wherein each R⁴ is independently selected from: halo, —CN, —NO₂, C₁₋₆ alkyl, C₁₋₆ haloalkyl, 2 to 8 membered heteroalkyl, Q¹, —OR⁵, —S(O)ₓR⁵, —NR⁵R⁶, —C(O)R⁵, —OC(O)R⁵, —C(O)OR⁵, —C(O)NR⁵R⁶, —NR⁵SO₂R⁶, and —SO₂NR⁵R⁶.

9. The compound according to claim 1, wherein each R⁴ is independently selected from: halo, —CN, C₁₋₃ alkyl, —OC₁₋₃ alkyl, —C(O) C₁₋₃ alkyl, —C(O)NH₂, —C(O)NH(C₁₋₃ alkyl) and —C(O)N(C₁₋₃ alkyl)₂.

10. The compound according to claim 1, wherein Ring A is selected from:

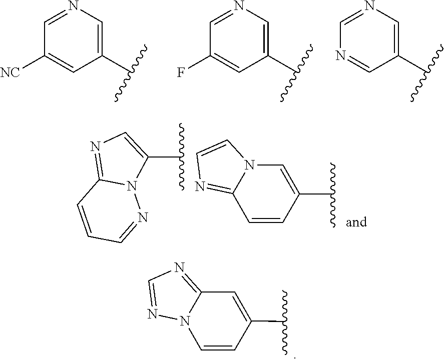

11. The compound according to claim 1, wherein the compound is a compound of the formula (III), or a pharmaceutically acceptable salt thereof: 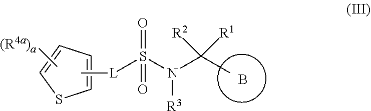
wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵(O)R⁶, —C(O)NR⁵R⁶, —NR⁵(O)OR⁶, —OC(O)NR⁵R⁶, —NR⁵SO₂R⁶, and —SO₂NR⁵R⁶,
wherein said C₁₋₆ alkyl, C₁₋₆ heteroalkyl, C₂₋₆ alkenyl and C₂₋₆ alkynyl is optionally substituted by one or more R⁷; and
a is an integer from 0 to 3.

12. The compound according to claim 1, wherein the compound is a compound of the formula (XI), or a pharmaceutically acceptable salt thereof: 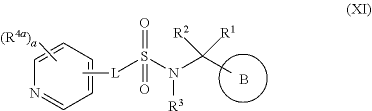
wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵(O)OR⁶, —OC(O)NR⁵R⁶, —NR⁵SO₂R⁶, and —SO₂NR⁵R⁶,
wherein said C₁₋₆ alkyl, 2 to 8 membered heteroalkyl, C₂₋₆ alkenyl and C₂₋₆ alkynyl is optionally substituted by one or more R⁷; and
a is an integer from 0 to 4.

13. The compound according to claim 1, wherein the compound is a compound of the formula (XIII), or a pharmaceutically acceptable salt thereof: 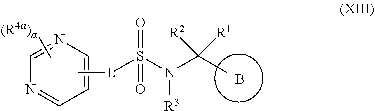
wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵(O)OR⁶, —OC(O)NR⁵R⁶, —NR⁵SO₂R⁶, and —SO₂NR⁵R⁶,
wherein said C₁₋₆ alkyl, 2 to 8 membered heteroalkyl, C₂₋₆ alkenyl and C₂₋₆ alkynyl is optionally substituted by one or more R⁷, and
a is an integer from 0 to 3.

14. The compound according to claim 1, wherein the compound is a compound of the formula (XV), or a pharmaceutically acceptable salt thereof: 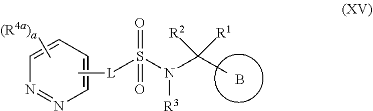
wherein:
each R^{4a} is independently selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
wherein said C₁₋₆ alkyl, 2 to 8 membered heteroalkyl, C₂₋₆ alkenyl and C₂₋₆ alkynyl is optionally substituted by one or more R⁷;
a is an integer from 0 to 3.

15. The compound according to claim 1, wherein the compound is a compound of the formula (XXI), or a pharmaceutically acceptable salt thereof: 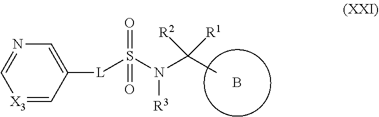
wherein:
X₃ is N or CR^{4a};
R^{4a} is selected from: halo, —CN, —NO₂, C₁₋₆ alkyl, C₁₋₆ haloalkyl, 2 to 8 membered heteroalkyl, C₂₋₆ alkenyl, C₂₋₆ alkynyl, Q¹, —OR⁵, —S(O)ₓR⁵, —NR⁵R⁶, —C(O)R⁵, —OC(O)R⁵, —C(O)OR⁵, —NR⁵C(O)R⁶, —C(O)NR⁵R⁶, —NR⁵C(O)OR⁶, —OC(O)NR⁵R⁶, —NR⁵SO₂R⁶, and —SO₂NR⁵R⁶,
wherein said C₁₋₆ alkyl, 2 to 8 membered heteroalkyl, C₂₋₆ alkenyl and C₂₋₆ alkynyl is optionally substituted by one or more R⁷.

16. The compound according to claim 15, wherein each R^{4a} is independently selected from: halo, —CN, C₁₋₃ alkyl, —OC₁₋₃ alkyl, —C(O) C₁₋₃ alkyl, —C(O)NH₂, —C(O)NH(C₁₋₃ alkyl) and —C(O)N(C₁₋₃ alkyl)₂.

17. The compound according to claim 15, wherein the group of the formula: 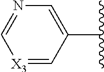
is selected from:

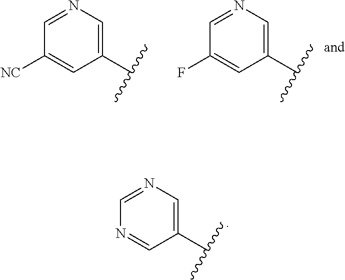 [SMILES: *c1cncc(C#N)c1 ; *c1cncc(F)c1 ; *c1cncnc1]

18. The compound according to claim 1, wherein Ring B is phenyl optionally substituted by one or more R¹⁰.

19. The compound according to claim 1, wherein Ring B is selected from:

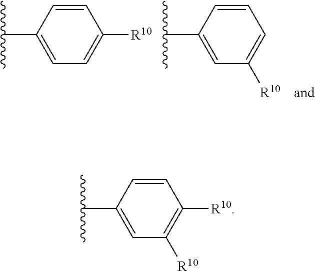

20. The compound according to claim 1, wherein Ring B is

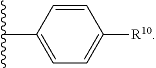

21. The compound according to claim 1, wherein each R¹⁰ is independently selected from: halo, C₁₋₃ alkyl, C₁₋₃ haloalkyl, —OC₁₋₃ alkyl and —OC₁₋₃ haloalkyl.

22. The compound according to claim 1, wherein Ring B is selected from:

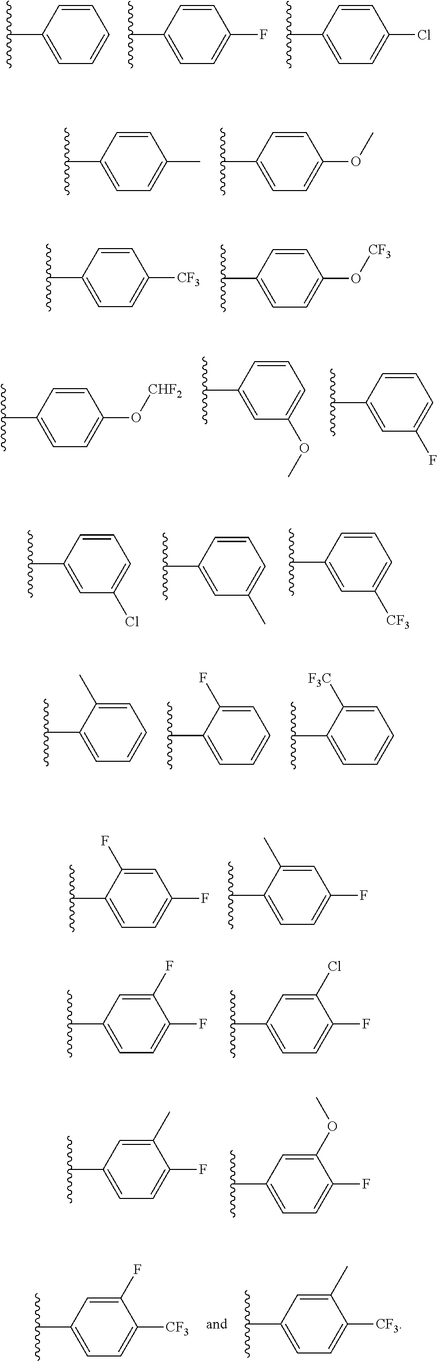

23. The compound according to claim 1, wherein Ring B is selected from:

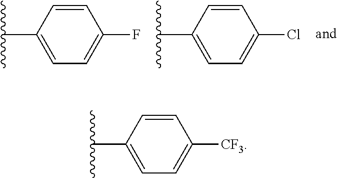

24. The compound according to claim 1, wherein Ring B is 4-fluorophenyl.

25. The compound according to claim 1, wherein L is selected from a bond and —CH₂—.

26. The compound according to claim 1, wherein L is a bond.

27. The compound according to claim 1, wherein R³ is selected from methyl, —CD₃, ethyl, and 2-fluoroethyl.

28. The compound according to claim 1, wherein R³ is selected from methyl and ethyl.

29. The compound according to claim 1, wherein R¹ is selected from C₁₋₆ alkyl and C₃₋₆ cycloalkyl, wherein R¹ is substituted by at least one fluorine.

30. The compound according to claim 1, wherein R¹ is selected from CH₂F, —CHF₂, and —CF₃.

31. The compound according to claim 1, wherein R¹ is —CF₃.

32. The compound according to claim 1, wherein R² is selected from H and methyl.

33. The compound according to claim 1, wherein R² is H.

34. The compound according to claim 1, wherein the group of the formula:

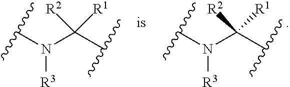

35. The compound according to claim 1, wherein the group of the formula:

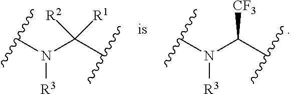

36. The compound according to claim 1, wherein the compound is selected from Compound List 1 in the description, or a pharmaceutically acceptable salt thereof.

37. A pharmaceutical composition comprising a compound according to claim 1, except that Compounds A and B are not excluded, or a pharmaceutically acceptable salt thereof, and a pharmaceutically acceptable excipient.

38. (canceled)

39. (canceled)

40. A method of treating a disease or medical disorder mediated by Cav2.3 in a subject in need thereof, the method comprising administering to the subject an effective amount of a compound according to claim 1, except that Compounds A and B are not excluded, or a pharmaceutically acceptable salt thereof.

41.-46. (canceled)
